(19) United States
(12) Patent Application Publication
(10) Pub. No.: US 2026/0234115 A1
(43) Pub. Date: Aug. 13, 2026

(54) LIPID COMPOUNDS, COMPOSITIONS, AND USES THEREOF

(71) Applicant: Liberate Bio, Inc., Boston, MA (US)

(72) Inventors: Gregory Lawrence HAMILTON, Atlanta, GA (US); Can SARISOZEN, Westford, MA (US); Parminder Kaur MANKOO, Foster City, CA (US); Stephen S. SCULLY, Concord, MA (US)

(21) Appl. No.: 19/487,299

(22) PCT Filed: May 29, 2024
(86) PCT No.: PCT/US2024/031498

§ 371 Date: Nov. 24, 2025

Related U.S. Application Data
(60) Provisional application No. 63/504,967, filed on May 30, 2023.

(51) Int. Cl. C07D 295/13; A61K 9/51; C07C 271/20

(52) U.S. Cl. CPC C07D 295/13; A61K 9/5123; C07C 271/20

(57) ABSTRACT

The present disclosure provides lipid compounds and compositions (e.g., lipid nanoparticle (LNP) compositions) comprising lipid compounds of the present disclosure. The present disclosure provides methods of delivering an active agent (e.g., polynucleotide) to a cell or tissue in a subject, preferably an extrahepatic cell or tissue, comprising administering to the subject an effective amount of a lipid nanoparticle of the present disclosure, wherein the lipid nanoparticle comprises lipid compounds of the present disclosure and the active agent (e.g., polynucleotide).

### RELATED APPLICATIONS

This application claims priority to U.S. Provisional Application No. 63/504,967, filed May 30, 2023, the entire contents of which are hereby incorporated by reference in their entirety.

### FIELD OF THE DISCLOSURE

The present disclosure provides lipid compounds and compositions (e.g., lipid nanoparticle (LNP) compositions) comprising lipid compounds of the present disclosure. The present disclosure also provides methods of delivering an active agent (e.g., polynucleotide) to a cell or tissue in a subject, such as an extrahepatic cell or tissue, comprising administering to the subject an effective amount of a lipid nanoparticle of the present disclosure, wherein the lipid nanoparticle comprises lipid compounds of the present disclosure and the active agent (e.g., polynucleotide).

### BACKGROUND

Many biologically active agents can be difficult to deliver into cells. These active agents include polynucleotide-based therapies (such as DNA-based therapies or RNA-based therapies like mRNA or siRNA), as well as CRISPR/Cas9-based gene editing therapies. Recently, lipid nanoparticles (LNPs) have been developed as encapsulating vehicles for delivering these types of biologically active agents to cells and tissues of interest. For example, LNPs comprising ionizable lipids can serve as vehicles for delivering biologically active agents across cell membranes and directly into target cells and for directing the active agents to preferred tissues of interest.

However, LNPs may benefit from improvement, such as extended stability and cellular or tissue specificity. For example, many LNPs strongly favor hepatic cells and tissues, and have limited effectiveness in targeting extrahepatic (i.e., non-hepatic) cells and tissues. A need therefore exists for stable LNPs compositions that effectively target extrahepatic cells and tissues in a subject.

### SUMMARY

The present disclosure provides lipid compounds and compositions (e.g., lipid nanoparticle (LNP) compositions) comprising lipid compounds of the present disclosure that may provide, inter alia, improved stability and/or extrahepatic targeting.

The present disclosure provides lipid compounds of the following formula:

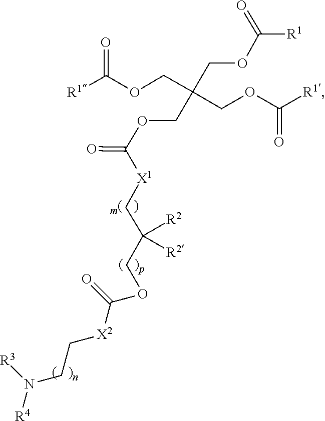

or a pharmaceutically acceptable salt thereof; wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R¹³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl, optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH—(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl, optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹;
R² is C₁-C₁₂ alkyl, C₂-C₁₂ alkenyl, C₂-C₁₂ alkynyl, C₁-C₁₂ alkoxy, (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R^{2′} is hydrogen, C₁-C₁₂ alkyl, alkenyl, or alkynyl, C₁-C₁₂ alkoxy, (C₁-C₄ alkyl)-(C₁-C₄ alkoxy);
wherein R² and R^{2′} can combine to form an optionally substituted C₄-C₆ cycloalkyl, an optionally substituted C₃-C₆ cycloalkyl, or an optionally substituted C₃-C₆ heterocycle;
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹¹ is hydrogen or C₁-C₆ alkyl, or wherein R¹¹ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl, or an optionally substituted C₅-C₆ aryl;
R¹⁴ is hydrogen or C₁-C₆ alkyl, or wherein R¹⁴ and R² join together to form an optionally substituted C₅-C₈ cycloalkyl;
m is 1-4;
p is 0-4; and
n is 1-5.

The present disclosure provides lipid compounds of the following formula:

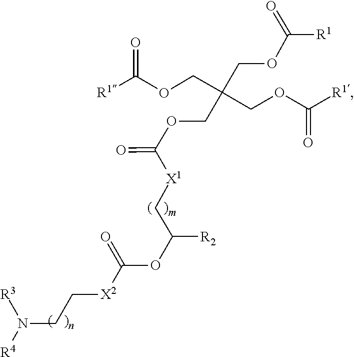

or a pharmaceutically acceptable salt thereof, wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl, optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl, optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹¹;
R² is C₁-C₁₂ alkyl, C₂-C₁₂ alkenyl, or C₂-C₁₂ alkynyl, C₁-C₁₂ alkoxy, (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl, or an optionally substituted C₅-C₆ aryl;
R¹¹ is hydrogen or C₁-C₆ alkyl, or wherein R¹¹ and R⁴ join together to form a heterocyclic ring comprising one or more N, O, or S heteroatoms;
R¹⁴ is hydrogen or C₁-C₆ alkyl, or wherein R¹⁴ and R² join together to form an optionally substituted C₅-C₈ cycloalkyl;
n is 1-5; and
m is 1-4.

In certain embodiments, two of R¹, R^{1′}, and R^{1″} are each independently (C₁-C₉ alkyl)-R⁵, and each R⁵ is independently C₂-C₁₂ alkenyl. In certain embodiments, each of R¹, R^{1′}, and R^{1″} is independently (C₁-C₉ alkyl)-R⁵; and each R⁵ is independently C₂-C₁₂ alkenyl. In certain embodiments, one of R¹, R^{1′}, and R^{1″} is (C₁-C₉ alkyl)-R^{5′}; and R^{5′} is C₆-C₁₀ cycloalkyl. In certain embodiments, R^{5′} is 1-adamantyl or 2-adamantyl. In certain embodiments, one of R¹, R^{1′}, and R^{1″} is CH(R⁷)R⁸; and each R⁷ and R⁸ is independently C₇-C₁₂ alkoxy.

In certain embodiments, two of R¹, R^{1′}, and R^{1″} are independently (C₁-C₉ alkyl)-R⁵; each R⁵ is independently C(O)O—R⁶; and each R⁶ is independently C₇-C₁₂ alkyl or C₇-C₁₂ alkenyl. In certain embodiments, each of R¹, R^{1′}, and R^{1″} is independently (C₁-C₉ alkyl)-R⁵; each R⁵ is independently C(O)O—R⁶; and each R⁶ is independently C₇-C₁₂ alkyl or C₇-C₁₂ alkenyl.

In certain embodiments, one of R¹, R^{1′}, and R^{1″} is (C₁-C₉ alkyl)-R^{5′}; and R^{5′} is C₆-C₁₀ cycloalkyl. In certain embodiments, R^{5′} is 1-adamantyl or 2-adamantyl.

In certain embodiments, one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy.

In certain embodiments, one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

In certain embodiments, R¹ and R^{1′} are (C₁-C₉ alkyl)-R^{5′}, R^{5′} is C(O)O—R⁶, and each R⁶ is independently C₇-C₁₂ alkyl or C₇-C₁₂ alkenyl; and R^{1′} is R¹²—R¹³, wherein R¹² is a bond or an optionally substituted, branched or unbranched C₁-C₆ alkyl, C₁-C₆ alkenyl, or C₁-C₆ alkynyl; and R¹³ is hydrogen, an optionally substituted C₃-C₁₂ cycloalkyl, or an optionally substituted C₅-C₆ aryl.

In certain embodiments, R¹² is a bond. In certain embodiments, R¹² is C₁-C₆ alkyl. In certain embodiments, R¹² is C₁ alkyl. In certain embodiments, R¹² is an optionally substituted, branched or unbranched C₁-C₆ alkyl, C₁-C₆ alkenyl, or C₁-C₆ alkynyl. In certain embodiments, R¹² is selected from pentyl, 1-methylpentyl, 4-methylpentyl, 5,5,5-trifluoropentyl, 4,4,5,5,5-pentafluoropentyl, and pent-4-ynyl.

In certain embodiments, R¹³ is hydrogen.

In certain embodiments, R¹³ is an optionally substituted C₃-C₁₂ cycloalkyl. In certain embodiments, R¹³ is selected from optionally substituted cyclopropane, optionally substituted cyclobutane, and optionally substituted cyclohexane such as 4-pentylcyclohexyl.

In certain embodiments, R¹³ is an optionally substituted fused C₃-C₁₂ cycloalkyl, an optionally substituted bridged C₃-C₁₂ cycloalkyl, or an optionally substituted spiro C₃-C₁₂ cycloalkyl.

In certain embodiments, R¹³ is an optionally substituted C₅-C₆ aryl. In certain embodiments, R¹³ is an optionally substituted phenyl such as 4-pentylphenyl or 3,5-di-tert-butylphenyl.

In certain embodiments, R¹³ is an optionally substituted bicyclo[2.2.2]pentane. In certain embodiments, R¹³ is an unsubstituted bicyclo[2.2.2]pentane, 1-(trifluoromethyl)bicyclo[1.1.1]pentane, or 1-methylbicyclo[1.1.1]pentane.

In certain embodiments, R¹³ is an optionally substituted bicyclo[2.1.0]pentane. In certain embodiments, R¹³ is an unsubstituted bicyclo[2.1.0]pentane.

In certain embodiments, R¹³ is an optionally substituted bicyclo[3.1.0]hexane. In certain embodiments, R¹³ is 6,6-difluorobicyclo[3.1.0]hexane.

In certain embodiments, R¹³ is an optionally substituted bicyclo[2.1.1]hexane. In certain embodiments, R¹³ is unsubstituted bicyclo[2.1.1]hexane or 1-fluorobicyclo[2.1.1]hexane.

In certain embodiments, R¹³ is an optionally substituted spiro[2.3]hexane. In certain embodiments, R¹³ is an optionally unsubstituted spiro[2.3]hexane or 1,1-difluorospiro[2.3]hexane.

In certain embodiments, R¹³ is an optionally substituted 1,1′-bi(cyclohexane).

In certain embodiments, R¹³ is an optionally substituted decahydronaphthalene.

In certain embodiments, R¹³ is an optionally substituted bicyclo[2.2.1]heptane. In certain embodiments, R¹³ is an unsubstituted bicyclo[2.2.1]heptane or 7,7-dimethylbicyclo[2.2.1]heptane.

In certain embodiments, R¹³ is an optionally substituted bicyclo[4.1.0]heptane. In certain embodiments, R¹³ is an unsubstituted bicyclo[4.1.0]heptane or 7,7-difluorobicyclo[4.1.0]heptane.

In certain embodiments, R¹³ is an optionally substituted bicyclo[3.2.0]heptane. In certain embodiments, R¹³ is an unsubstituted bicyclo[3.2.0]heptane.

In certain embodiments, R¹³ is optionally substituted spiro[3.3]heptane. In certain embodiments, R¹³ is an unsubstituted spiro[3.3]heptane or 2,2-difluorospiro[3.3]heptane.

In certain embodiments, R¹³ is an optionally substituted bicyclo[2.2.2]octane. In certain embodiments, R¹³ is an unsubstituted bicyclo[2.2.2]octane or 1-methylbicyclo[2.2.2]octane.

In certain embodiments, R¹³ is an optionally substituted bicyclo[3.2.1]octane. In certain embodiments, R¹³ is an unsubstituted bicyclo[3.2.1]octane or 8-oxabicyclo[3.2.1]octane.

In certain embodiments, R¹³ is an optionally substituted spiro[2.5]octane. In certain embodiments, R¹³ is an unsubstituted spiro[2.5]octane or 1,1-difluorospiro[2.5]octane.

In certain embodiments, R¹³ is an optionally substituted bicyclo[3.2.2]nonane. In certain embodiments, R¹³ is an unsubstituted bicyclo[3.2.2]nonane or 1-fluorobicyclo[3.2.2]nonane.

In certain embodiments, R¹³ is an optionally substituted 1-bicyclo[3.3.1]nonane. In certain embodiments, R¹³ is an unsubstituted bicyclo[3.3.1]nonane or 1-methylbicyclo[3.3.1]nonane.

In certain embodiments, R¹³ is adamantane.

In certain embodiments, two of R¹, R^{1′}, and R^{1″} are independently (C₁-C₉ alkyl)-R⁵; each R⁵ is independently OC(O)CH(R⁹)R¹⁰; and each R⁹ and R¹⁰ is independently C₁-C₁₂ alkyl or C₂-C₁₂ alkenyl. In certain embodiments, each of R¹, R^{1′}, and R^{1″} is independently (C₁-C₉ alkyl)-R⁵; R⁵ is OC(O)CH(R⁹)R¹⁰; and each R⁹ and R¹⁰ is independently C₁-C₁₂ alkyl or C₂-C₁₂ alkenyl.

In certain embodiments, one of R¹, R^{1′}, and R^{1″} is (C₁-C₉ alkyl)-R^{5′}; R^{5′} is C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl. In certain embodiments, two of R¹, R^{1′}, and R^{1″} are independently (C₁-C₉ alkyl)-R⁵; each R⁵ is independently C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl. In certain embodiments, each of R¹, R^{1′}, and R^{1″} is independently (C₁-C₉ alkyl)-R⁵; R⁵ is C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl.

In certain embodiments, one of R¹, R^{1′}, and R^{1″} is (C₁-C₉ alkyl)-R^{5′}; and R^{5′} is C₆-C₁₀ cycloalkyl; optionally wherein R^{5′} is 1-adamantyl or 2-adamantyl. In certain embodiments, one of R¹, R^{1′}, and R^{1″} is (C₁-C₉ alkyl)-R^{5′}; R^{5′} is C(O)O—R⁶; and R⁶ is C₇-C₁₂ alkyl or C₇-C₁₂ alkenyl. In certain embodiments, one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy.

In certain embodiments, two of R¹, R^{1′}, and R^{1″} are independently (C₁-C₉ alkyl)-R⁵; each R⁵ is independently CH(R⁷)R⁸; and each R⁷ and R⁸ is independently C₇-C₁₂ alkoxy. In certain embodiments, each of R¹, R^{1′}, and R^{1″} is independently (C₁-C₉ alkyl)-R⁵; each R⁵ is independently CH(R⁷)R⁸; and each R⁷ and R⁸ is independently C₇-C₁₂ alkoxy.

In certain embodiments, one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

In certain embodiments, R² is C₁-C₁₂ alkyl, C₂-C₁₂ alkenyl, or C₂-C₁₂ alkynyl. In certain embodiments, R² is C₄-C₈ alkyl. In certain embodiments, R² is methyl, ethyl, propyl, isopropyl, butyl, 1-isobutyl, 2-isobutyl, tert-butyl, C₅ alkyl, C₆ alkyl, C₈ alkyl, or C₁₀ alkyl.

In certain embodiments, R² is C₁-C₁₂ alkoxy or (C₁-C₄ alkyl)-(C₁-C₄ alkoxy). In certain embodiments, R² is methoxy, ethoxy, methoxymethyl, or ethoxyethyl.

In certain embodiments, R² is optionally substituted C₃-C₁₂ cycloalkyl, optionally substituted C₃-C₆ heterocycle, or optionally substituted C₅-C₆ aryl. In certain embodiments, R² is optionally substituted cyclopropyl, cyclobutyl, cyclopentyl, or cyclohexyl such as 4-pentylcyclohexyl.

In certain embodiments, R² is optionally substituted phenyl. In certain embodiments, R² is 4-pentylphenyl.

In certain embodiments, R² is optionally substituted (C₁-C₄ alkyl)-(optionally substituted C₃-C₁₂ cycloalkyl), (C₁-C₄ alkyl)-(optionally substituted C₃-C₆ heterocycle), or (C₁-C₄ alkyl)-(optionally substituted C₅-C₆ aryl). In certain embodiments, R² is —CH₂-cyclopropyl, —(CH₂)₂-cyclopropyl, —CH₂-cyclohexyl, —(CH₂)₂-cyclohexyl, —(CH₂)₂-(4-pentylcyclohexyl), —CH₂-phenyl, or —(CH₂)₂-phenyl.

In certain embodiments, R^{2′} is hydrogen. In certain embodiments, R^{2′} is an C₁-C₁₂ alkyl, C₂-C₁₂ alkenyl, or C₂-C₁₂ alkynyl. In certain embodiments, R² is methoxy, ethoxy, methoxymethyl, or ethoxyethyl.

In certain embodiments, R² and R^{2′} combine to form an optionally substituted C₄-C₆ cycloalkyl or C₄-C₆ heterocycle. In certain embodiments, R² and R^{2′} combine to form cyclohexane or pyran.

In certain embodiments, X¹ is CH₂. In certain embodiments, X¹ is CHR¹⁴, and R¹⁴ and R² join together to form an optionally substituted C₅-C₈ cycloalkyl. In certain embodiments, R¹⁴ and R² join together to form an optionally substituted C₅ cycloalkyl or optionally substituted C₆ cycloalkyl.

In certain embodiments, X² is NH. In certain embodiments, X² is O. In certain embodiments, R³ and R⁴ are each independently C₁-C₆ alkyl.

In certain embodiments, R³ and R⁴ are joined together to form a heterocyclic ring comprising a nitrogen heteroatom. In certain embodiments, R³ and R⁴ are joined together to form pyrrolidine. In certain embodiments, R³ and R⁴ are joined together with a preceding alkyl group to form quinuclidine.

In certain embodiments, n is 1, 2, 3, or 4.

In certain embodiments, m is 1, 2, 3 or 4.

In certain embodiments, p is 0, 1, 2, 3 or 4.

In certain embodiments, two of R¹, R^{1′}, and R^{1″} are independently (C₂ alkyl)-R⁵.

In certain embodiments, two of R¹, R^{1′}, and R^{1″} are independently (C₃ alkyl)-R⁵.

In certain embodiments, two of R¹, R^{1′}, and R^{1″} are independently (C₄ alkyl)-R⁵.

In certain embodiments, two of R¹, R^{1′}, and R^{1″} are independently (C₅ alkyl)-R⁵.

In certain embodiments, two of R¹, R^{1′}, and R^{1″} are independently (C₆ alkyl)-R⁵.

In certain embodiments, two of R¹, R^{1′}, and R^{1″} are independently (C₇ alkyl)-R⁵.

In certain embodiments, two of R¹, R^{1′}, and R^{1″} are independently (C₈ alkyl)-R⁵.

In certain embodiments, the compound is of the following formula:

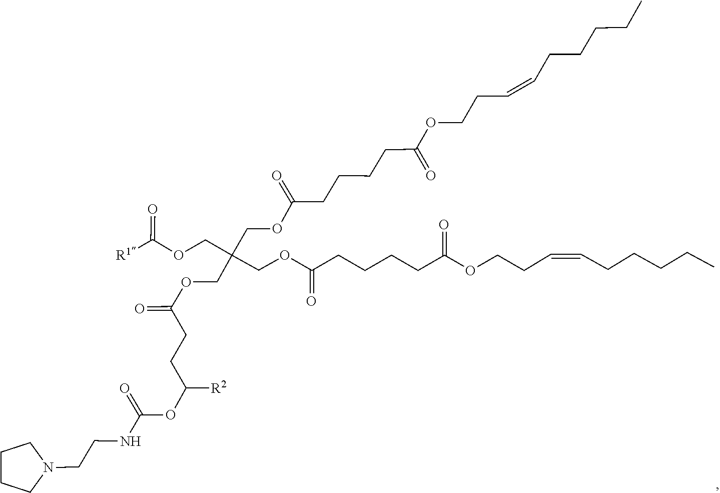

or a pharmaceutically acceptable salt thereof.

In certain embodiments, the compound is of the following formula:

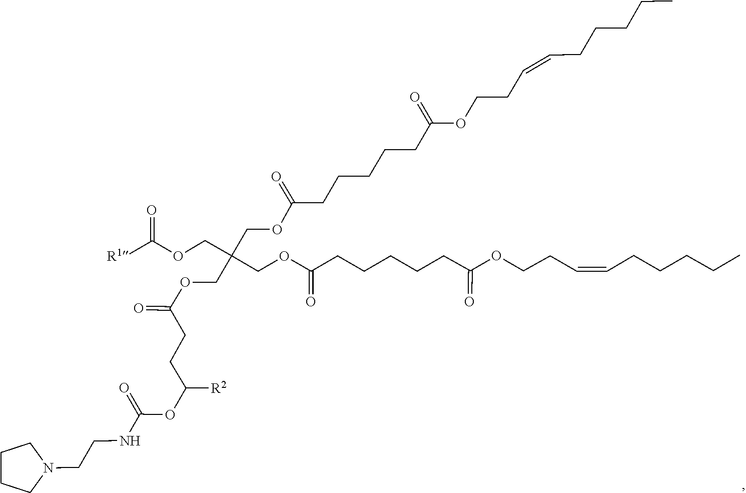

or a pharmaceutically acceptable salt thereof.

In certain embodiments, the compound is of the following formula:

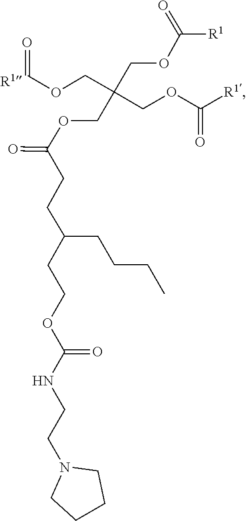

or a pharmaceutically acceptable salt thereof.

In certain embodiments, the compound is any one of Compounds 1-209 or a pharmaceutically acceptable salt thereof.

In certain embodiments, the compound is any one of Compounds 7, 8, 10, 13, 14, 26, 33, 38, 39, 40, 48, 60, 61, 89, 103, or 109, or a pharmaceutically acceptable salt thereof. In certain embodiments, the compound is Compound 7 or a pharmaceutically acceptable salt thereof. In certain embodiments, the compound is Compound 8 or a pharmaceutically acceptable salt thereof. In certain embodiments, the compound is Compound 10 or a pharmaceutically acceptable salt thereof. In certain embodiments, the compound is Compound 13 or a pharmaceutically acceptable salt thereof. In certain embodiments, the compound is Compound 14 or a pharmaceutically acceptable salt thereof. In certain embodiments, the compound is Compound 26 or a pharmaceutically acceptable salt thereof. In certain embodiments, the compound is Compound 33 or a pharmaceutically acceptable salt thereof. In certain embodiments, the compound is Compound 38 or a pharmaceutically acceptable salt thereof. In certain embodiments, the compound is Compound 39 or a pharmaceutically acceptable salt thereof. In certain embodiments, the compound is Compound 40 or a pharmaceutically acceptable salt thereof. In certain embodiments, the compound is Compound 48 or a pharmaceutically acceptable salt thereof. In certain embodiments, the compound is Compound 60 or a pharmaceutically acceptable salt thereof. In certain embodiments, the compound is Compound 61 or a pharmaceutically acceptable salt thereof. In certain embodiments, the compound is Compound 89 or a pharmaceutically acceptable salt thereof. In certain embodiments, the compound is Compound 103 or a pharmaceutically acceptable salt thereof. In certain embodiments, the compound is Compound 109 or a pharmaceutically acceptable salt thereof.

In certain embodiments, the present disclosure provides a lipid nanoparticle comprising a compound (e.g., lipid compound) of the present disclosure. In certain embodiments, the present disclosure provides a lipid nanoparticle comprising a compound (e.g., lipid compound) of the present disclosure; a phospholipid; a cholesterol; and a polyethylene glycol lipid. In certain embodiments, the lipid nanoparticle comprises: about 20-80 mol % of a compound (e.g., lipid compound) of the present disclosure, about 7.5-40 mol % of phospholipid, about 6-45 mol % of cholesterol, and about 1-4 mol % of PEG lipid. In certain embodiments, the lipid nanoparticle comprises: about 45-65 mol % of a compound (e.g., lipid compound) of the present disclosure, about 10 mol % of phospholipid, about 25-45 mol % of cholesterol, and about 1-4 mol % of PEG lipid. In certain embodiments, the lipid nanoparticle comprises: from about 45-50 mol % of a compound (e.g., lipid compound) of the present disclosure, about 10 mol % of phospholipid, about 38-42 mol % of cholesterol, and from about 2-3 mol % of PEG lipid. In certain embodiments, the lipid nanoparticle comprises: about 47.5 mol % of a compound (e.g., lipid compound) of the present disclosure, about 40 mol % of cholesterol, and about 2.5 mol % of PEG lipid. In certain embodiments, the lipid nanoparticle comprises: about 47.5-52.5 mol % of a compound (e.g., lipid compound) of the present disclosure, about 10 mol % of phospholipid, about 37-40 mol % of cholesterol, and about 1-2 mol % of PEG lipid. In certain embodiments, the lipid nanoparticle comprises: about 50 mol % of a compound (e.g., lipid compound) of the present disclosure, about 38.5 mol % of cholesterol, and about 1.5 mol % of PEG lipid. In certain embodiments, the lipid nanoparticle comprises: about 57.5-62.5 mol % of a compound (e.g., lipid compound) of the present disclosure, about 10 mol % of phospholipid, about 26-29 mol % of cholesterol, and about 2-3 mol % of PEG lipid. In certain embodiments, the lipid nanoparticle comprises: about 60 mol % of a compound (e.g., lipid compound) of the present disclosure, about 27.5 mol % of cholesterol, and about 2.5 mol % of PEG lipid. In certain embodiments, the lipid nanoparticle comprises: about 45-50 mol % of a compound (e.g., lipid compound) of the present disclosure, about 10 mol % of phospholipid, about 37.5-40.5 mol % of cholesterol, and about 3-4 mol % of PEG lipid. In certain embodiments, the lipid nanoparticle comprises: about 47.5 mol % of a compound (e.g., lipid compound) of the present disclosure, about 39 mol % of cholesterol, and about 3.5 mol % of PEG lipid.

In certain embodiments, the lipid nanoparticle comprises a targeting component. In certain embodiments, the targeting component is a targeting lipid. In certain embodiments, the targeting component is an active targeting component. In certain embodiments, the active targeting component is a protein, a peptide, a small molecule, or an antibody or antigen binding fragment thereof. In certain embodiments, the active targeting component is a protein. In certain embodiments, the active targeting component is a peptide. In certain embodiments, the active targeting component is a small molecule. In certain embodiments, the active targeting component is an antibody or antigen binding fragment thereof.

In certain embodiments, the lipid nanoparticle comprises one or more polynucleotides encapsulated within the lipid nanoparticle. In certain embodiments, the one or more polynucleotides comprises RNA. In certain embodiments, the one or more polynucleotides comprises DNA. In certain embodiments, the one or more polynucleotides comprises DNA and RNA.

In certain embodiments, the present disclosure provides a pharmaceutical composition comprising a lipid nanoparticle of the present disclosure, and a pharmaceutically acceptable excipient. In certain embodiments, the present disclosure provides a pharmaceutical composition comprising a lipid nanoparticle of the present disclosure, wherein the lipid nanoparticle comprises one or more polynucleotides encapsulated within the lipid nanoparticle; and a pharmaceutically acceptable excipient.

In certain embodiments, the present disclosure provides a method of delivering a polynucleotide to a cell or tissue in a subject, comprising administering to the subject an effective amount of a lipid nanoparticle or a pharmaceutical composition of the present disclosure. In certain embodiments, the present disclosure provides a method of delivering a polynucleotide to a cell or tissue in a subject, comprising administering to the subject an effective amount of a lipid nanoparticle or a pharmaceutical composition of the present disclosure. In certain embodiments, the cell or tissue comprises an extrahepatic cell or tissue. In certain embodiments, the cell or tissue comprises a brain cell or tissue. In certain embodiments, the cell or tissue comprises a lung cell or tissue. In certain embodiments, the cell or tissue comprises a bone marrow cell or tissue. In certain embodiments, the cell or tissue comprises a spleen cell or tissue. In certain embodiments, the cell or tissue comprises a muscle cell or tissue. In certain embodiments, the cell or tissue comprises a kidney cell or tissue. In certain embodiments, the cell or tissue comprises a heart cell or tissue. In certain embodiments, the cell or tissue comprises a pancreas cell or tissue. In certain embodiments, the cell or tissue comprises an immune cell or tissue.

In certain embodiments, the present disclosure provides a method of treating a disease in a subject, comprising administering to the subject a therapeutically effective amount of a pharmaceutical composition of the present disclosure.

In certain embodiments, the present disclosure provides a method of producing a therapeutic composition, comprising encapsulating an active agent within a lipid nanoparticle, wherein the lipid nanoparticle comprises a compound (e.g., lipid compound) of the present disclosure. In certain embodiments, the active agent comprises DNA. In certain embodiments, the active agent comprises RNA. In certain embodiments, the active agent comprises DNA and RNA.

In certain embodiments, the present disclosure provides a method of producing a vaccine or prophylactic composition, the method comprising: encapsulating an active agent (e.g., RNA or DNA) within a lipid nanoparticle, wherein the lipid nanoparticle comprises a compound (e.g., lipid compound) of the present disclosure. In certain embodiments, the active agent comprises DNA. In certain embodiments, the active agent comprises RNA. In certain embodiments, the active agent comprises DNA and RNA.

In certain embodiments, the present disclosure provides the use of a lipid nanoparticle or a pharmaceutical composition of the present disclosure in the manufacture of a medicament for delivering a polynucleotide to an extrahepatic cell or tissue in a subject. In certain embodiments, the present disclosure provides the use of a lipid nanoparticle or a pharmaceutical composition of the present disclosure in the manufacture of a medicament for treating a disease in a subject.

### BRIEF DESCRIPTION OF THE DRAWINGS

FIG. 1A shows stability over four weeks of select lipid nanoparticles (LNPs) prepared according to Formulation F1, as measured by particle size. FIG. 1B shows stability over four weeks of select LNPs prepared according to Formulation F1, as measured by polydispersity index (PDI). N/P=molar ratio of ionizable nitrogen to phosphate groups. T=time. D=day. W=week.

FIG. 1C shows stability over two weeks of select LNPs prepared according to Formulation F3, as measured by particle size. FIG. 1D shows stability over two weeks of select LNPs prepared according to Formulation F3, as measured by polydispersity index (PDI). N/P=molar ratio of ionizable nitrogen to phosphate groups. T=time. D=day. W=week.

FIG. 2A, FIG. 2B, FIG. 2C, and FIG. 2D provide mouse organ luciferase imaging results after 6 hours following IV administration of select LNP compound pools. Results are shown as Total Flux [p/s].

FIG. 3 provides mouse brain luciferase imaging results after 6 hours following ICV administration of select LNP compound pools. Results are shown as Total Flux [p/s].

FIG. 4A and FIG. 4B provide mouse lung luciferase imaging results following intratracheal administration of LNP compound pools (Compounds 2, 3, 5, and 7). Results are shown as Total Flux [p/s]. FIG. 4A provides live animal imaging results and FIG. 4B provides ex vivo imaging results.

FIG. 4C provides mouse lung luciferase imaging results following intratracheal administration of LNP compound pools (Compounds 8, 9, 11, and 20). Results are shown as Total Flux [p/s].

FIG. 4D provides mouse lung luciferase imaging results following intratracheal administration of LNP compound pools (Compounds 12, 14, 16, and 18). Results are shown as Total Flux [p/s].

FIG. 5A provides mouse organ luciferase imaging results following IV administration of select LNP compounds prepared according to Formulation F2. Results are shown as Total Flux [p/s]. FIG. 5B shows these results normalized to MC3 activity.

FIG. 6A and FIG. 6B provide mouse lung and trachea luciferase imaging results following intratracheal administration of select LNP compounds prepared according to Formulation F2. Results are shown as Total Flux [p/s]. FIG. 6A provides live animal imaging results and FIG. 6B provides ex vivo imaging results.

FIG. 7A, FIG. 7B, and FIG. 7C provide mouse in vivo Activity Screening results (mean total flux) in the spleen, femur, and muscle after administration of certain LNP compounds of the disclosure to a group of mice.

FIG. 8A, FIG. 8B, and FIG. 8C provide NHP in vivo Activity Screening results (unique molecular identifier counts) in the spleen, femur, and muscle after administration of certain LNP compounds of the disclosure to a group of cynomolgus monkeys.

FIG. 9 provide NHP in vivo Activity Screening results (unique molecular identifier counts) in the spleen after administration of certain LNP compounds of the disclosure to a group of cynomolgus monkeys.

FIG. 10 provide NHP in vivo Activity Screening results (unique molecular identifier counts) in the femur after administration of certain LNP compounds of the disclosure to a group of cynomolgus monkeys.

FIG. 11 provide NHP in vivo Activity Screening results (unique molecular identifier counts) in the muscle after administration of certain LNP compounds of the disclosure to a group of cynomolgus monkeys.

### DETAILED DESCRIPTION

### I. Lipid Compounds

Without being bound by theory, the lipid compounds disclosed herein facilitate delivery of an active agent to a desired target in a subject, e.g., to extrahepatic cells or tissues in the subject, when the active agent is encapsulated in an LNP comprising said lipid compound(s).

In certain embodiments, the present disclosure provides lipid compounds of Formula (I):

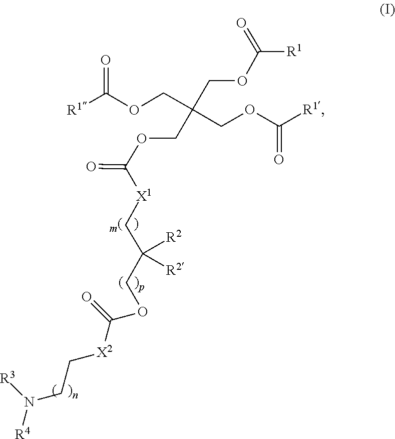

or a pharmaceutically acceptable salt thereof; wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH—(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹¹;
R² is C₁-C₁₂ alkyl, C₂-C₁₂ alkenyl, C₂-C₁₂ alkynyl, C₁-C₁₂ alkoxy, or (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R^{2′} is hydrogen, C₁-C₁₂ alkyl, alkenyl, or alkynyl, C₁-C₁₂ alkoxy, (C₁-C₄ alkyl)-(C₁-C₄ alkoxy);
wherein R² and R^{2′} can combine to form an optionally substituted C₄-C₆ cycloalkyl, an optionally substituted C₃-C₆ cycloalkyl, or an optionally substituted C₃-C₆ heterocycle;
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹¹ is hydrogen or C₁-C₆ alkyl, or wherein R¹¹ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl (including fused, bridged, or spiro cycloalkyl), or an optionally substituted C₅-C₆ aryl;
R¹⁴ is hydrogen or C₁-C₆ alkyl, or wherein R¹⁴ and R² join together to form an optionally substituted C₅-C₈ cycloalkyl;
m is 1-4;
p is 0-4; and
n is 1-5.

In certain embodiments, the present disclosure provides lipid compounds of Formula (II):

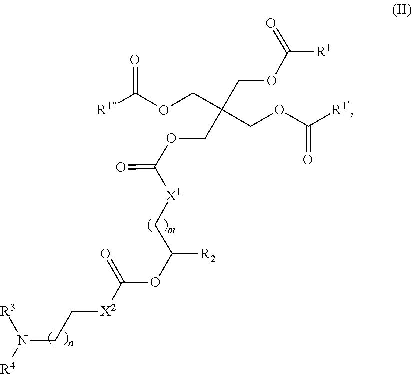

or a pharmaceutically acceptable salt thereof; wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R¹³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹¹;
R² is C₁-C₁₂ alkyl, C₂-C₁₂ alkenyl, C₂-C₁₂ alkynyl, C₁-C₁₂ alkoxy, or (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹¹ is hydrogen or C₁-C₆ alkyl, or wherein R¹¹ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl (including fused, bridged, or spiro cycloalkyl), or an optionally substituted C₅-C₆ aryl;
R¹⁴ is hydrogen or C₁-C₆ alkyl, or wherein R¹⁴ and R² join together to form an optionally substituted C₅-C₈ cycloalkyl;
n is 1-5; and
m is 1-4.

In certain embodiments, each of R¹, R^{1′}, and R^{1″} is independently: (C₁-C₉ alkyl)-R⁵, (C₂-C₉ alkenyl)-R⁵, (C₂-C₉ alkynyl)-R⁵, or (C₁-C₈ alkoxy)-R⁵. In certain embodiments, all three of R¹, R^{1′}, and R^{1″} are the same. In certain embodiments, two of R¹, R^{1′}, and R^{1″} are the same. In certain embodiments, all three of R¹, R^{1′}, and R^{1″} are different.

In certain embodiments, at least one of R¹, R^{1′}, and R^{1″} is (C₂ alkyl)-R⁵. In certain embodiments, two of R¹, R^{1′}, and R^{1″} are independently (C₂ alkyl)-R⁵. In certain embodiments, all three of R¹, R^{1′}, and R^{1″} are (C₂ alkyl)-R⁵. In certain embodiments, at least one of R¹, R^{1′}, and R^{1″} is (C₃ alkyl)-R⁵. In certain embodiments, two of R¹, R^{1′}, and R^{1″} are independently (C₃ alkyl)-R⁵. In certain embodiments, all three of R¹, R^{1′}, and R^{1″} are (C₃ alkyl)-R⁵. In certain embodiments, at least one of R¹, R^{1′}, and R^{1″} is (C₄ alkyl)-R⁵. In certain embodiments, two of R¹, R^{1′}, and R^{1″} are independently (C₄ alkyl)-R⁵. In certain embodiments, all three of R¹, R^{1′}, and R^{1″} are (C₄ alkyl)-R⁵. In certain embodiments, at least one of R¹, R^{1′}, and R^{1″} is (C₅ alkyl)-R⁵. In certain embodiments, two of R¹, R^{1′}, and R^{1″} are independently (C₅ alkyl)-R⁵. In certain embodiments, all three of R¹, R^{1′}, and R^{1″} are (C₅ alkyl)-R⁵. In certain embodiments, at least one of R¹, R^{1′}, and R^{1″} is (C₆ alkyl)-R⁵. In certain embodiments, two of R¹, R^{1′}, and R^{1″} are independently (C₆ alkyl)-R⁵. In certain embodiments, all three of R¹, R^{1′}, and R^{1″} are (C₆ alkyl)-R⁵. In certain embodiments, at least one of R¹, R^{1′}, and R^{1″} is (C₇ alkyl)-R⁵. In certain embodiments, two of R¹, R^{1′}, and R^{1″} are independently (C₇ alkyl)-R⁵. In certain embodiments, all three of R¹, R^{1′}, and R^{1″} are (C₈ alkyl)-R⁵. In certain embodiments, at least one of R¹, R^{1′}, and R^{1″} is (C₈ alkyl)-R⁵. In certain embodiments, two of R¹, R^{1′}, and R^{1″} are independently (C₈ alkyl)-R⁵. In certain embodiments, all three of R¹, R^{1′}, and R^{1″} are (C₈ alkyl)-R⁵.

In certain embodiments, each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰. In certain embodiments, all R⁵ groups are the same. In certain embodiments, two R⁵ groups are the same. In certain embodiments, all R⁵ groups are different.

In certain embodiments, each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl (including fused, bridged, or spiro cycloalkyl), optionally substituted C₅-C₆ aryl, 1-adamantyl, 2-adamantyl, or sterolyl. In certain embodiments, each R⁷ and R⁸ is independently: C₇-C₁₂ alkyl, C₇-C₁₂ alkenyl, C₇-C₁₂ alkynyl, or C₇-C₁₂ alkoxy. In certain embodiments, each R⁹ and R¹⁰ is independently: C₁-C₁₂ alkyl or C₂-C₁₂ alkenyl.

In certain embodiments, at least one of R¹, R^{1′}, and R^{1″} is (C₁-C₉ alkyl)-R⁵, and R⁵ is C₂-C₁₂ alkenyl. In certain embodiments, two of R¹, R^{1′}, and R^{1″} are each independently (C₁-C₉ alkyl)-R⁵, and each R⁵ is independently C₂-C₁₂ alkenyl. In certain embodiments, all three of R¹, R^{1′}, and R^{1″} are independently (C₁-C₉ alkyl)-R⁵; and each R⁵ is independently C₂-C₁₂ alkenyl. In certain embodiments, one of R¹, R^{1′}, and R^{1″} is (C₁-C₉ alkyl)-R^{5′}; and R^{5′} is C₆-C₁₀ cycloalkyl. In certain embodiments, R^{5′} is 1-adamantyl or 2-adamantyl. In certain embodiments, one of R¹, R^{1′}, and R^{1″} is CH(R⁷)R⁸; and each R⁷ and R⁸ is independently C₇-C₁₂ alkoxy.

In certain embodiments, at least one of R¹, R^{1′}, and R^{1″} is (C₁-C₉ alkyl)-R⁵; R⁵ is C(O)O—R⁶; and R⁶ is C₇-C₁₂ alkyl or C₇-C₁₂ alkenyl. In certain embodiments, two of R¹, R^{1′}, and R^{1″} are independently (C₁-C₉ alkyl)-R⁵; each R⁵ is independently C(O)O—R⁶; and each R⁶ is independently C₇-C₁₂ alkyl or C₇-C₁₂ alkenyl. In certain embodiments, all three of R¹, R^{1′}, and R^{1″} are independently (C₁-C₉ alkyl)-R⁵; each R⁵ is independently C(O)O—R⁶; and each R⁶ is independently C₇-C₁₂ alkyl or C₇-C₁₂ alkenyl. In certain embodiments, one of R¹, R^{1′}, and R^{1″} is (C₁-C₉ alkyl)-R^{5′}; and R^{5′} is C₆-C₁₀ cycloalkyl. In certain embodiments, R^{5′} is 1-adamantyl or 2-adamantyl. In certain embodiments, one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy. In certain embodiments, one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

In certain embodiments, at least one of R¹, R^{1′}, and R^{1″} is (C₁-C₉ alkyl)-R⁵; R⁵ is OC(O)CH(R⁹)R¹⁰; and R⁹ and R¹⁰ are each independently C₁-C₁₂ alkyl or C₂-C₁₂ alkenyl. In certain embodiments, two of R¹, R^{1′}, and R^{1″} are independently (C₁-C₉ alkyl)-R⁵; each R⁵ is independently OC(O)CH(R⁹)R¹⁰; and each R⁹ and R¹⁰ is independently C₁-C₁₂ alkyl or C₂-C₁₂ alkenyl. In certain embodiments, all three of R¹, R^{1′}, and R^{1″} are independently (C₁-C₉ alkyl)-R⁵; each R⁵ is OC(O)CH(R⁹)R¹⁰; and each R⁹ and R¹⁰ is independently C₁-C₁₂ alkyl or C₂-C₁₂ alkenyl. In certain embodiments, one of R¹, R^{1′}, and R^{1″} is (C₁-C₉ alkyl)-R^{5′}; and R^{5′} is C₆-C₁₀ cycloalkyl (e.g., R^{5′} is 1-adamantyl or 2-adamantyl). In certain embodiments, one of R¹, R^{1′}, and R^{1″} is (C₁-C₉ alkyl)-R^{5′}; R^{5′} is C(O)O—R⁶; and R⁶ is C₇-C₁₂ alkyl or C₇-C₁₂ alkenyl. In certain embodiments, one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy.

In certain embodiments, R¹ and R^{1′} are (C₁-C₉ alkyl)-R^{5′}, R^{5′} is C(O)O—R⁶, and each R⁶ is independently C₇-C₁₂ alkyl or C₇-C₁₂ alkenyl; and R^{1′} is R¹²—R¹³, wherein R¹² is a bond or an optionally substituted, branched or unbranched C₁-C₆ alkyl, C₁-C₆ alkenyl, or C₁-C₆ alkynyl; R¹³ is hydrogen, an optionally substituted C₃-C₁₂ cycloalkyl (including fused, bridged, or spiro cycloalkyl), or an optionally substituted C₅-C₆ aryl.

In certain embodiments, R¹² is a bond.

In certain embodiments, R¹² is an optionally substituted, branched or unbranched C₁-C₆ alkyl, C₁-C₆ alkenyl, or C₁-C₆ alkynyl. In certain embodiments, R¹² is a C₁ alkyl. In certain embodiments, R¹² is an optionally substituted, branched C₁-C₆ alkyl. In certain embodiments, R¹² is pentyl. In certain embodiments, R¹² is 1-methylpentyl. In certain embodiments, R¹² is 4-methylpentyl. In certain embodiments, R¹² is 5,5,5-trifluoropentyl. In certain embodiments, R¹² is 4,4,5,5,5-pentafluoropentyl. In certain embodiments, R¹² is an optionally substituted, branched C₁-C₆ alkynyl. In certain embodiments, R¹² is pent-4-ynyl.

In certain embodiments, R¹³ is hydrogen.

In certain embodiments, R¹³ is an optionally substituted C₃-C₁₂ cycloalkyl (including fused, bridged, or spiro cycloalkyl). In certain embodiments, R¹³ is an optionally substituted cyclopropane. In certain embodiments, R¹³ is an optionally substituted cyclobutane. In certain embodiments, R¹³ is an optionally substituted cyclohexane. In certain embodiments, R¹³ is 4-pentylcyclohexyl. In certain embodiments, R¹³ is an optionally substituted C₅-C₆ aryl. In certain embodiments, R¹³ is an optionally substituted phenyl. In certain embodiments, R¹³ is 4-pentylphenyl. In certain embodiments, R¹³ is 3,5-di-tert-butylphenyl.

In certain embodiments, R¹³ is an optionally substituted, fused C₃-C₁₂ cycloalkyl. In certain embodiments, R¹³ is an optionally substituted, bridged C₃-C₁₂ cycloalkyl. In certain embodiments, R¹³ is an optionally substituted, spiro C₃-C₁₂ cycloalkyl. In certain embodiments, R¹³ is an optionally substituted bicyclo[2.2.2]pentane, such unsubstituted as bicyclo[2.2.2]pentane, 1-(trifluoromethyl)bicyclo[1.1.1]pentane, or 1-methylbicyclo[1.1.1]pentane. In certain embodiments, R¹³ is an optionally substituted bicyclo[2.1.0]pentane, such unsubstituted as bicyclo[2.2.2]pentane. In certain embodiments, R¹³ is an optionally substituted bicyclo[3.1.0]hexane, such as unsubstituted bicyclo[3.1.0]hexane or 6,6-difluorobicyclo[3.1.0]hexane. In certain embodiments, R¹³ is an optionally substituted bicyclo[2.1.1]hexane, such as unsubstituted bicyclo[2.1.1]hexane or 1-fluorobicyclo[2.1.1]hexane. In certain embodiments, R¹³ is an optionally substituted spiro[2.3]hexane, such as unsubstituted spiro[2.3]hexane or 1,1-difluorospiro[2.3]hexane. In certain embodiments, R¹³ is an optionally substituted 1,1′-bi(cyclohexane). In certain embodiments, R¹³ is an optionally substituted decahydronaphthalene. In certain embodiments, R¹³ is an optionally substituted bicyclo[2.2.1]heptane, such as unsubstituted bicyclo[2.2.1]heptane or 7,7-dimethylbicyclo[2.2.1]heptane. In certain embodiments, R¹³ is an optionally substituted bicyclo[4.1.0]heptane, such as unsubstituted bicyclo[4.1.0]heptane or 7,7-difluorobicyclo[4.1.0]heptane. In certain embodiments, R¹³ is an optionally substituted bicyclo[3.2.0]heptane, such as unsubstituted bicyclo[3.2.0]heptane. In certain embodiments, R¹³ is an optionally substituted spiro[3.3]heptane, such as unsubstituted spiro[3.3]heptane or 2,2-difluorospiro[3.3]heptane. In certain embodiments, R¹³ is an optionally substituted bicyclo[2.2.2]octane, such as unsubstituted bicyclo[2.2.2]octane or 1-methylbicyclo[2.2.2]octane. In certain embodiments, R¹³ is an optionally substituted bicyclo[3.2.1]octane, such as unsubstituted bicyclo[3.2.1]octane or 8-oxabicyclo[3.2.1]octane. In certain embodiments, R¹³ is an optionally substituted spiro[2.5]octane, such as unsubstituted spiro[2.5]octane or 1,1-difluorospiro[2.5]octane. In certain embodiments, R¹³ is an optionally substituted bicyclo[3.2.2]nonane, such as unsubstituted bicyclo[3.2.2]nonane or 1-flurorbicyclo[3.2.2]nonane. In certain embodiments, R¹³ is an optionally substituted bicyclo[3.3.1]nonane, such as unsubstituted bicyclo[3.3.1]nonane or 1-methylbicyclo[3.3.1]nonane. In certain embodiments, R¹³ is adamantane.

In certain embodiments, at least one of R¹, R^{1′}, and R^{1″} is (C₁-C₉ alkyl)-R⁵; R⁵ is CH(R⁷)R⁸; and R⁷ and R⁸ are independently C₇-C₁₂ alkoxy. In certain embodiments, two of R¹, R^{1′}, and R^{1″} are independently (C₁-C₉ alkyl)-R⁵; each R⁵ is independently CH(R⁷)R⁸; and each R⁷ and R⁸ is independently C₇-C₁₂ alkoxy. In certain embodiments, each of R¹, R^{1′}, and R^{1″} is independently (C₁-C₉ alkyl)-R⁵; each R⁵ is independently CH(R⁷)R⁸; and each R⁷ and R⁸ is independently C₇-C₁₂ alkoxy. In certain embodiments, one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

In certain embodiments, at least one of R¹, R^{1′}, and R^{1″} is (C₁-C₉ alkyl)-R⁵; R⁵ is C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl. In certain embodiments, two of R¹, R^{1′}, and R^{1″} are independently (C₁-C₉ alkyl)-R⁵; each R⁵ is independently C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl.

In certain embodiments, X¹ is O, NH, CHR¹⁴; and R¹⁴ is hydrogen or C₁-C₆ alkyl. In certain embodiments, X¹ is CH₂. In certain embodiments, X¹ is NH. In certain embodiments, X¹ is O. In certain embodiments, X¹ is CHR¹⁴, and R¹⁴ and R² join together to form an optionally substituted C₅-C₈ cycloalkyl. In certain embodiments, R¹⁴ and R² join together to form an optionally substituted C₅ cycloalkyl. In certain embodiments, R¹⁴ and R² join together to form an optionally substituted C₆ cycloalkyl.

In certain embodiments, X² is O, NH, or CHR¹¹; and R¹¹ is hydrogen or C₁-C₆ alkyl. In certain embodiments, X² is CHR¹¹, and R¹¹ and R⁴ join together to form a heterocyclic ring comprising one or more N, O, or S heteroatoms. In certain embodiments, R¹¹ and R⁴ join together to form 1-methylpyrrolidine (with R³ as a methyl group). In certain embodiments, R¹¹ and R⁴ join together to form 1-methylpiperidine (with R³ as a methyl group).

In certain embodiments, X² is NH. In certain embodiments, X² is O.

In certain embodiments, R² is C₁-C₁₂ alkyl, C₂-C₁₂ alkenyl, C₂-C₁₂ alkynyl, C₁-C₁₂ alkoxy, (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl).

In certain embodiments, R² is C₁-C₁₂ alkyl, C₂-C₁₂ alkenyl, C₂-C₁₂ alkynyl. In certain embodiments, R² is C₁-C₁₂ alkyl. In certain embodiments, R² is C₄-C₈ alkyl. In certain embodiments, R² is methyl. In certain embodiments, R² is trifluoromethyl. In certain embodiments, R² is ethyl. In certain embodiments, R² is propyl or isopropyl. In certain embodiments, R² is butyl, 1-isobutyl, 2-isobutyl, or tert-butyl. In certain embodiments, R² is C₅ alkyl. In certain embodiments, R² is C₆ alkyl. In certain embodiments, R² is C₈ alkyl. In certain embodiments, R² is C₁₀ alkyl.

In certain embodiments, R² is C₁-C₁₂ alkoxy. In certain embodiments, R² is (C₁-C₄ alkyl)-(C₁-C₄ alkoxy). In certain embodiments, R² is methoxymethyl. In certain embodiments, R² is ethoxyethyl.

In certain embodiments, R² is optionally substituted C₃-C₁₂ cycloalkyl, optionally substituted C₃-C₆ heterocycle, or optionally substituted C₅-C₆ aryl. In certain embodiments, R² is optionally substituted cyclopropyl. In certain embodiments, R² is optionally substituted cyclobutyl. In certain embodiments, R² is optionally substituted cyclopentyl. In certain embodiments, R² is optionally substituted cyclohexyl. In certain embodiments, R² is 4-pentylcyclohexyl. In certain embodiments, R² is optionally substituted phenyl. In certain embodiments, R² is 4-pentylphenyl.

In certain embodiments, R² is (C₁-C₄ alkyl)-(optionally substituted C₃-C₁₂ cycloalkyl), (C₁-C₄ alkyl)-(optionally substituted C₃-C₆ heterocycle), or (C₁-C₄ alkyl)-(optionally substituted C₅-C₆ aryl). In certain embodiments, R² is —CH₂-cyclopropyl, —(CH₂)₂-cyclopropyl, —CH₂-cyclohexyl, —(CH₂)₂-cyclohexyl, —CH₂-phenyl, or —(CH₂)₂-phenyl.

In certain embodiments, R^{2′} is hydrogen. In certain embodiments, R^{2′} is a C₁-C₁₂ alkyl, C₂-C₁₂ alkenyl, or C₂-C₁₂ alkynyl. In certain embodiments, R^{2′} is methyl. In certain embodiments, R^{2′} is ethyl. In certain embodiments, R^{2′} is propyl. In certain embodiments, R^{2′} is butyl. In certain embodiments, R^{2′} is C₅ alkyl. In certain embodiments, R^{2′} is C₆ alkyl. In certain embodiments, R^{2′} is C₁-C₁₂ alkoxy. In certain embodiments, R^{2′} is (C₁-C₄ alkyl)-(C₁-C₄ alkoxy). In certain embodiments, R^{2′} is methoxymethyl. In certain embodiments, R^{2′} is ethoxyethyl. In certain embodiments, R^{2′} is the same as R².

In certain embodiments, R² and R^{2′} combine to form an optionally substituted C₄-C₆ cycloalkyl. In certain embodiments, R² and R^{2′} combine to form an optionally substituted cyclohexane. In certain embodiments, R² and R^{2′} combine to form an optionally substituted C₄-C₆ heterocycle. In certain embodiments, R² and R^{2′} combine to form an optionally substituted pyran.

In certain embodiments, R³ and R⁴ are each independently: hydrogen, C₁-C₆ alkyl, C₁-C₆ alkoxy, or C₁-C₆ hydroxyalkyl. In certain embodiments, R³ and R⁴ are each independently C₁-C₆ alkyl. In certain embodiments, R³ and R⁴ join together to form a heterocyclic ring comprising one or more N, O, or S heteroatoms. In certain embodiments, R³ and R⁴ are joined together to form a heterocyclic ring comprising a nitrogen heteroatom. In certain embodiments, R³ and R⁴ are joined together to form pyrrolidine. In certain embodiments, R³ and R⁴ are joined together with a preceding alkyl group to form quinuclidine.

In certain embodiments, n is 1-5. In certain embodiments, n is 1. In certain embodiments, n is 2. In certain embodiments, n is 3. In certain embodiments, n is 4. In certain embodiments, n is 5.

In certain embodiments, m is 1-4. In certain embodiments, m is 1. In certain embodiments, m is 2. In certain embodiments, m is 3. In certain embodiments, m is 4.

In certain embodiments, p is 0-4. In certain embodiments, p is 0. In certain embodiments, p is 1. In certain embodiments, p is 2. In certain embodiments, p is 3. In certain embodiments, p is 4.

Examples of lipid compounds of Formula (I) and Formula (II) are presented in Table 1. Pharmaceutically acceptable salts of the compounds presented in Table 1 are also encompassed.

TABLE 1
Exemplary Compounds

| Compound | |
|---|---|
| Number | Compound |
| 1 | 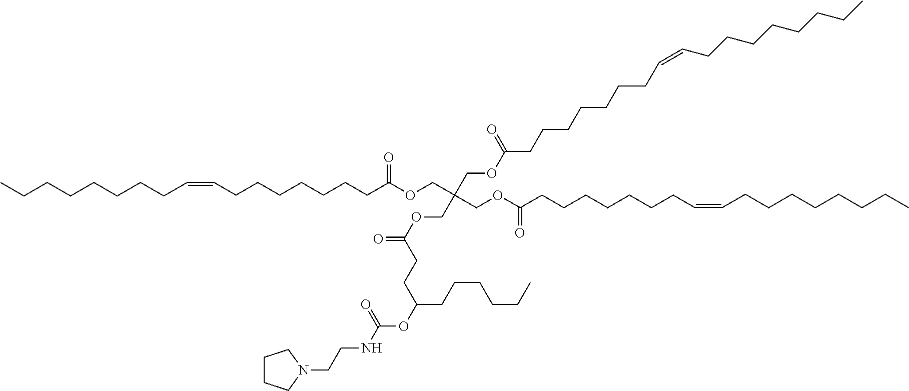 [SMILES: CCCCCCCC/C=C\CCCCCCCC(=O)OCC(COC(=O)CCCCCCC/C=C\CCCCCCCC)(COC(=O)CCCCCCC/C=C\CCCCCCCC)COC(=O)CCC(CCCCCC)OC(=O)NCCN1CCCC1] |
| 2 | 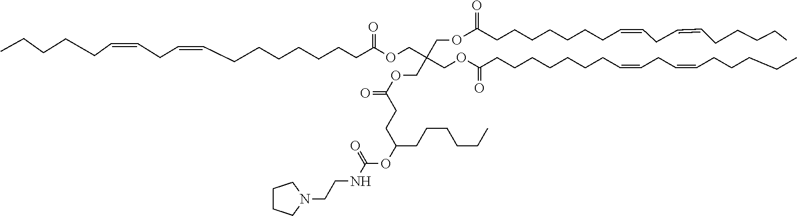 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)COC(=O)CCC(CCCCCC)OC(=O)NCCN1CCCC1] |
| 3 | 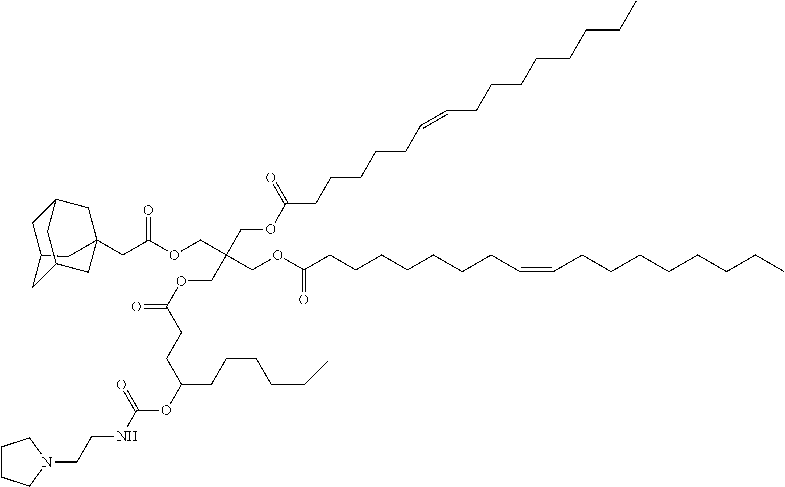 [SMILES: CCCCCCCC/C=C\CCCCCCCC(=O)OCC(COC(=O)CCCCC/C=C\CCCCCCCC)(COC(=O)CCC(CCCCCC)OC(=O)NCCN1CCCC1)COC(=O)CC12CC3CC(CC(C3)C1)C2] |
| 4 | 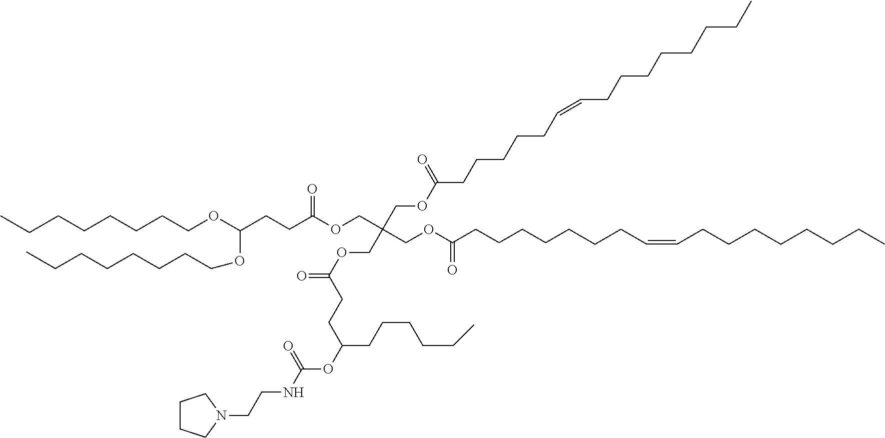 [SMILES: CCCCCCCC/C=C\CCCCCCCC(=O)OCC(COC(=O)CCCCC/C=C\CCCCCCCC)(COC(=O)CCC(CCCCCC)OC(=O)NCCN1CCCC1)COC(=O)CCC(OCCCCCCCC)OCCCCCCCC] |
| 5 | 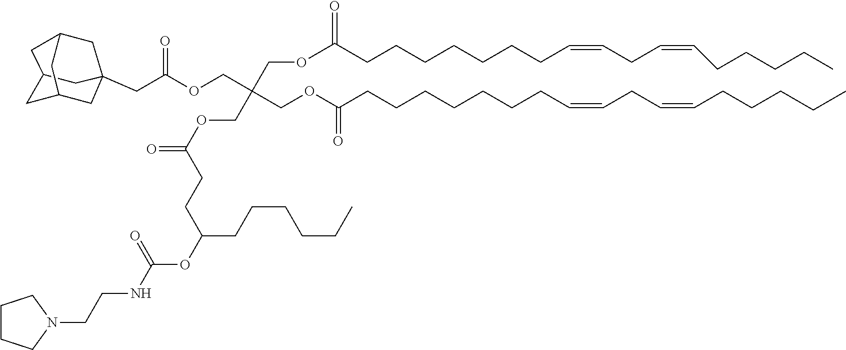 [SMILES: CCCCC/C=C\C/C=C\CCCCCCCC(=O)OCC(COC(=O)CCCCCCC/C=C\C/C=C\CCCCC)(COC(=O)CCC(CCCCCC)OC(=O)NCCN1CCCC1)COC(=O)CC12CC3CC(CC(C3)C1)C2] |
| 6 | 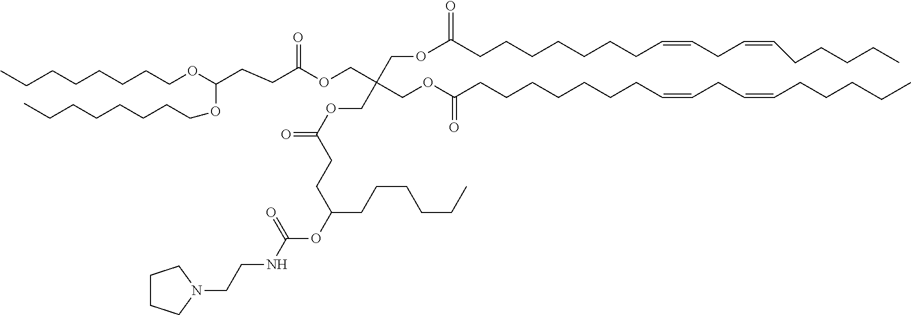 [SMILES: CCCCC/C=C\C/C=C\CCCCCCCC(=O)OCC(COC(=O)CCCCCCC/C=C\C/C=C\CCCCC)(COC(=O)CCC(CCCCCC)OC(=O)NCCN1CCCC1)COC(=O)CCC(OCCCCCCCC)OCCCCCCCC] |
| 7 | 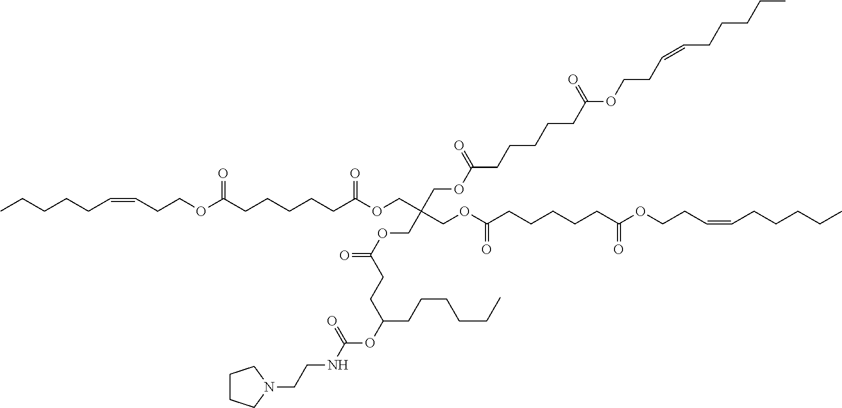 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCCCCC)OC(=O)NCCN1CCCC1] |
| 8 | 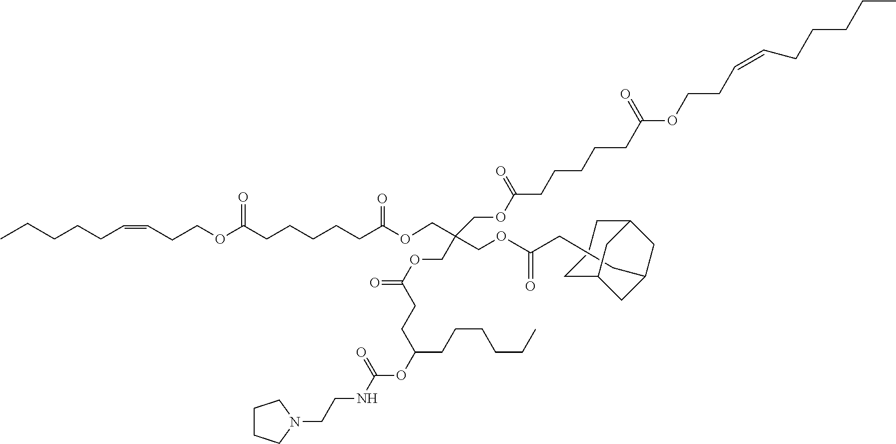 [SMILES: CCCCC/C=C\CCOC(=O)CCCCCC(=O)OCC(COC(=O)CCCCCC(=O)OCC/C=C\CCCCC)(COC(=O)CCC1CC2CCCC(C2)C1)COC(=O)CCC(CCCCCC)OC(=O)NCCN1CCCC1] |
| 9 | 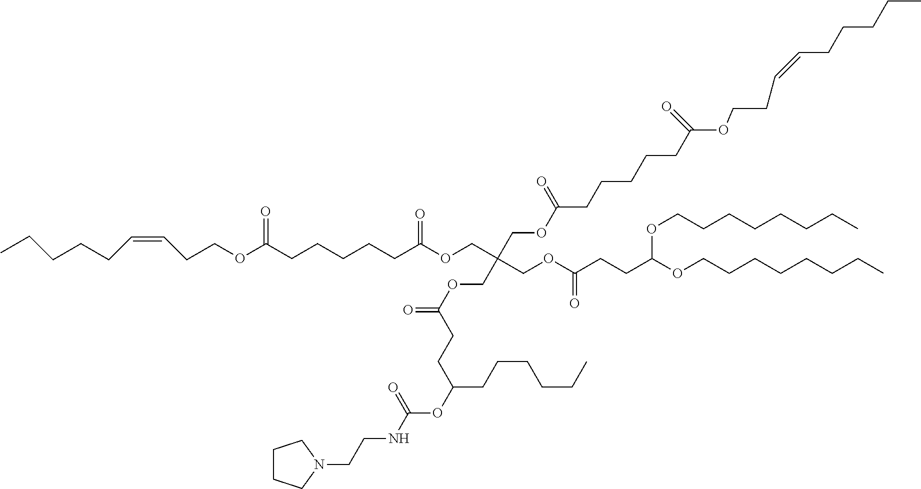 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)CCC(OCCCCCCCC)OCCCCCCCC] |
| 10 | 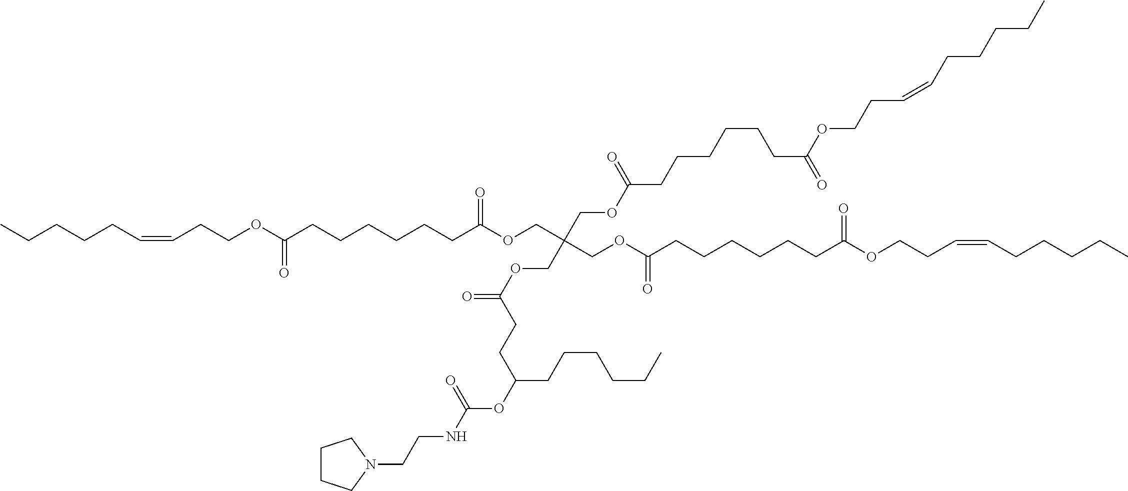 [SMILES: CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCCCC)OC(=O)NCCN1CCCC1] |
| 11 | 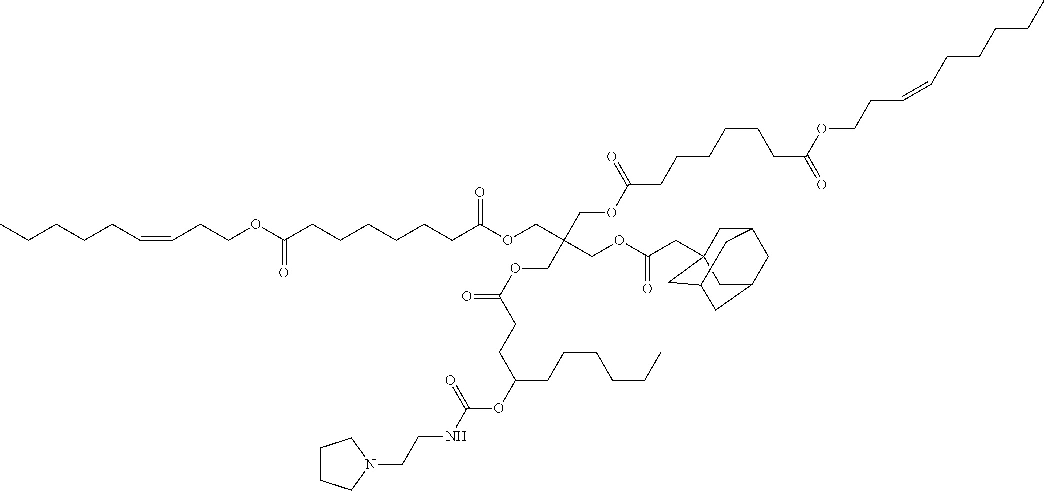 [SMILES: CCCCC/C=C\CCOC(=O)CCCCCCC(=O)OCC(COC(=O)CCCCCCC(=O)OCC/C=C\CCCCC)(COC(=O)CCC(CCCCCC)OC(=O)NCCN1CCCC1)COC(=O)CC12CC3CC(CC(C3)C1)C2] |
| 12 | 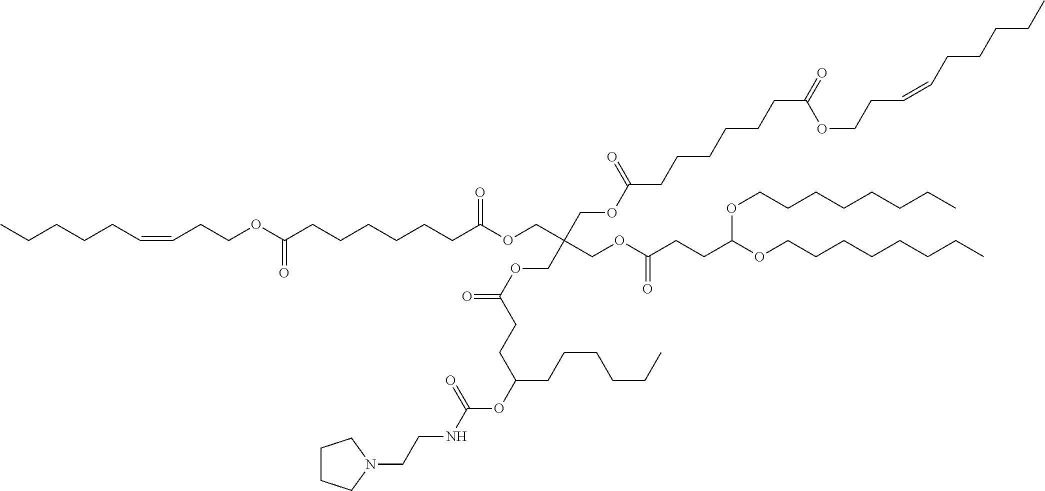 [SMILES: CCCCC/C=C\CCOC(=O)CCCCCCC(=O)OCC(COC(=O)CCCCCCC(=O)OCC/C=C\CCCCC)(COC(=O)CCC(CCCCCC)OC(=O)NCCN1CCCC1)COC(=O)CCC(OCCCCCCCC)OCCCCCCCC] |
| 13 | 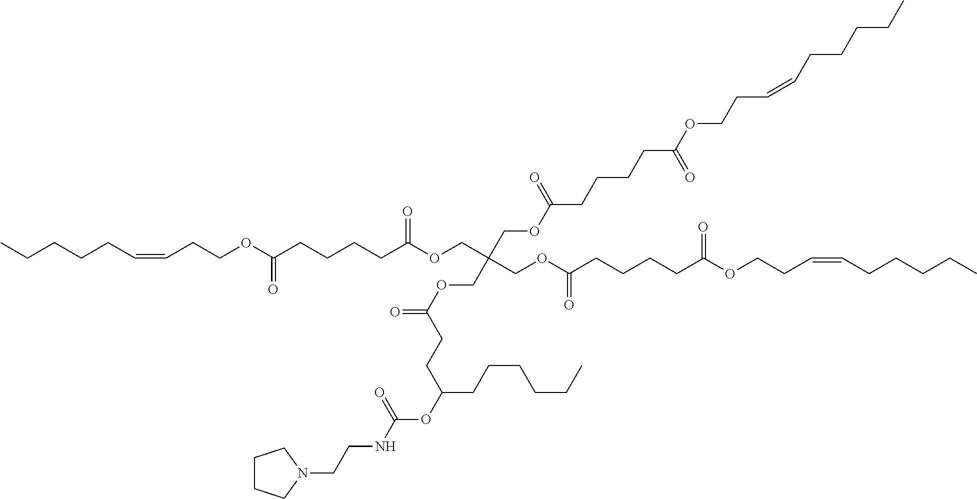 [SMILES: CCCCC/C=C\CCOC(=O)CCCCC(=O)OCC(COC(=O)CCCCC(=O)OCC/C=C\CCCCC)(COC(=O)CCCCC(=O)OCC/C=C\CCCCC)COC(=O)CCC(CCCCCC)OC(=O)NCCN1CCCC1] |
| 14 | 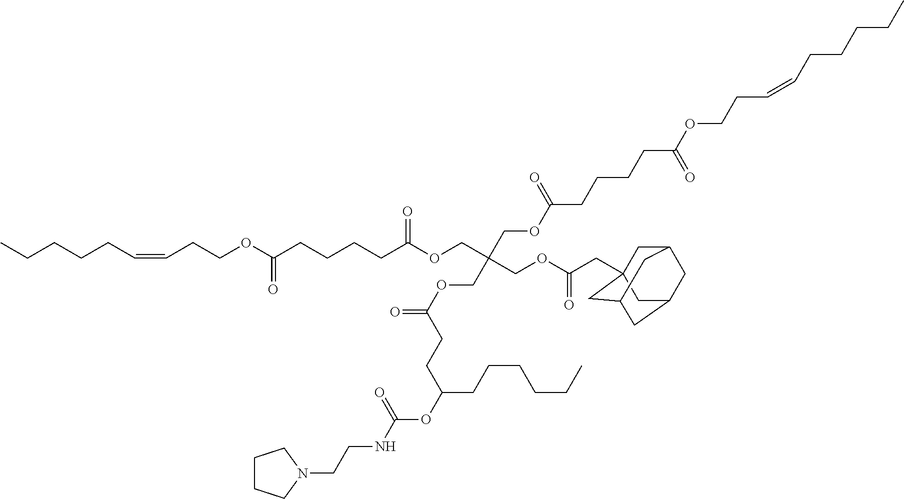 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC12CC3CC(CC(C3)C1)C2] |
| 15 | 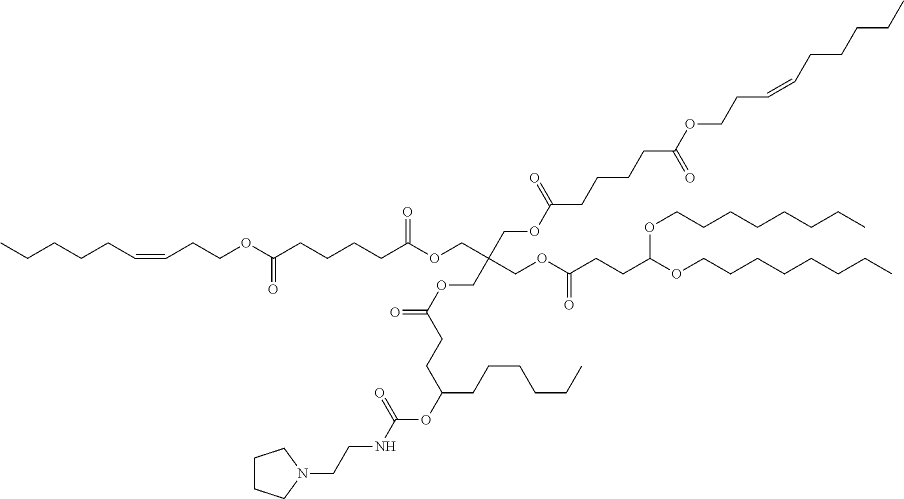 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CCC(OCCCCCCCC)OCCCCCCCC] |
| 16 | 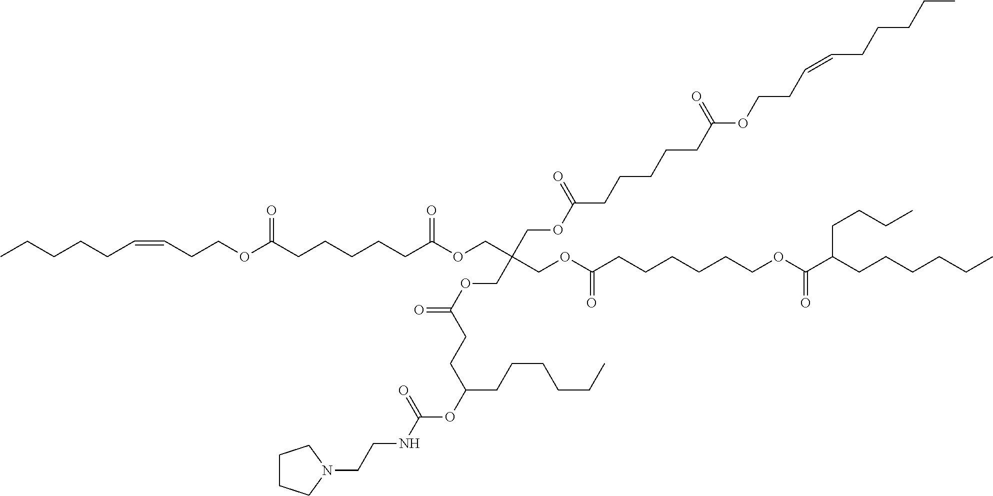 [SMILES: CCCCC/C=C\CCOC(=O)CCCCCC(=O)OCC(COC(=O)CCCCCCOC(=O)C(CCCC)CCCCCC)(COC(=O)CCCCCC(=O)OCC/C=C\CCCCC)COC(=O)CCC(CCCCCC)OC(=O)NCCN1CCCC1] |
| 17 | 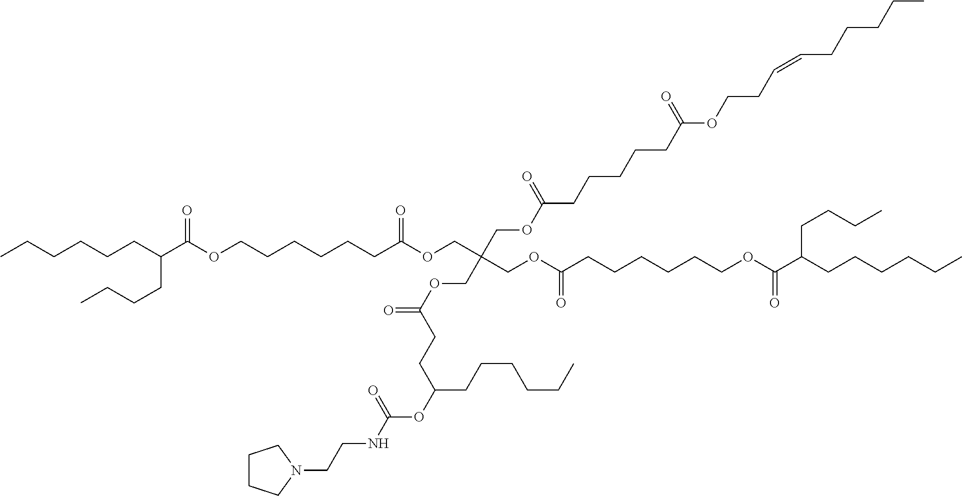 [SMILES: CCCCC/C=C\CCOC(=O)CCCCCC(=O)OCC(COC(=O)CCCCCCOC(=O)C(CCCC)CCCCCC)(COC(=O)CCCCCCOC(=O)C(CCCC)CCCCCC)COC(=O)CCC(CCCCCC)OC(=O)NCCN1CCCC1] |
| 18 | 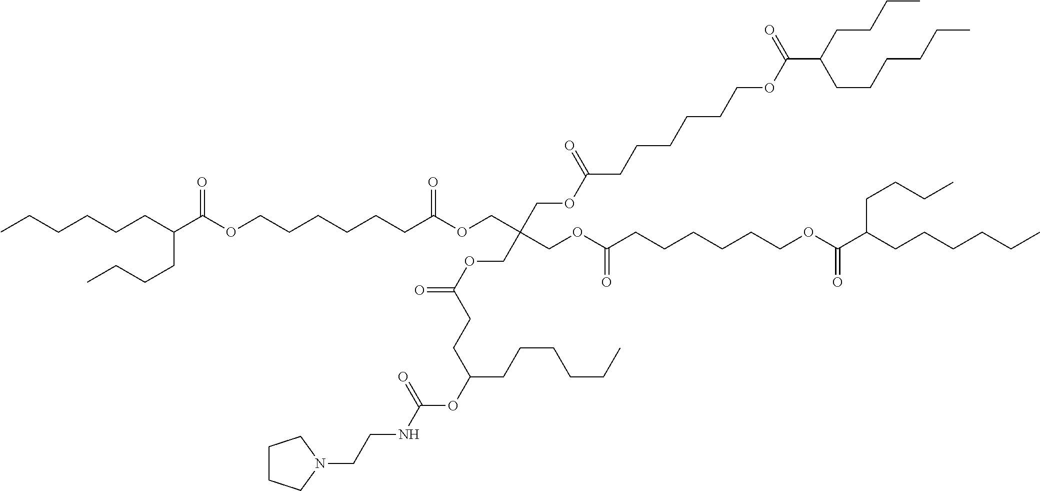 [SMILES: CCCCCCC(CCC(=O)OCC(COC(=O)CCCCCCOC(=O)C(CCCC)CCCCCC)(COC(=O)CCCCCCOC(=O)C(CCCC)CCCCCC)COC(=O)CCCCCCOC(=O)C(CCCC)CCCCCC)OC(=O)NCCN1CCCC1] |
| 19 | 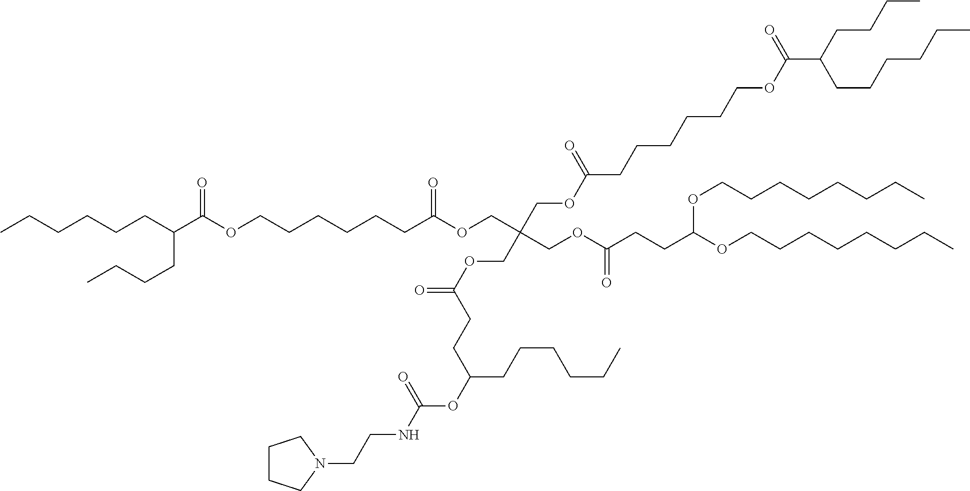 [SMILES: CCCCCCCCOC(CCC(=O)OCC(COC(=O)CCCCCCOC(=O)C(CCCC)CCCCCC)(COC(=O)CCCCCCOC(=O)C(CCCC)CCCCCC)COC(=O)CCC(CCCCCC)OC(=O)NCCN1CCCC1)OCCCCCCCC] |
| 20 | 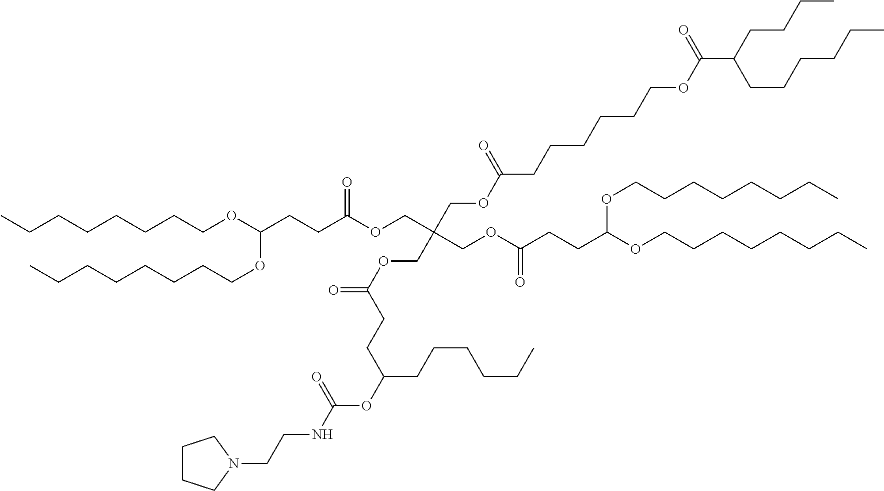 [SMILES: CCCCCCCCOC(CCC(=O)OCC(COC(=O)CCCCCCOC(=O)C(CCCC)CCCCCC)(COC(=O)CCC(CCCCCC)OC(=O)NCCN1CCCC1)COC(=O)CCC(OCCCCCCCC)OCCCCCCCC)OCCCCCCCC] |
| 21 | 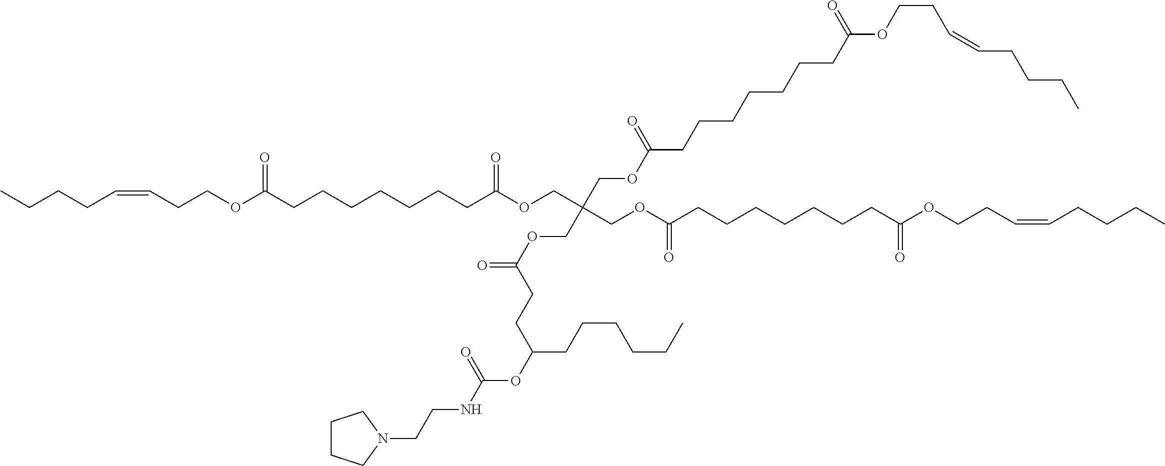 [SMILES: CCCC/C=C\CCOC(=O)CCCCCCCC(=O)OCC(COC(=O)CCCCCCCC(=O)OCC/C=C\CCCC)(COC(=O)CCCCCCCC(=O)OCC/C=C\CCCC)COC(=O)CCC(CCCCCC)OC(=O)NCCN1CCCC1] |
| 22 | 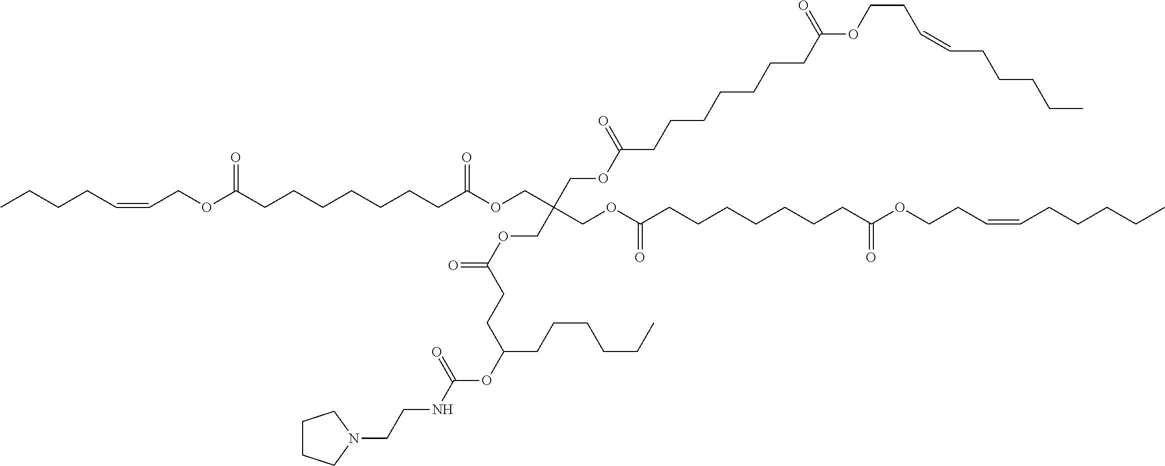 [SMILES: CCCC/C=C\COC(=O)CCCCCCCC(=O)OCC(COC(=O)CCCCCCCC(=O)OCC/C=C\CCCCC)(COC(=O)CCCCCCCC(=O)OCC/C=C\CCCCC)COC(=O)CCC(CCCCCC)OC(=O)NCCN1CCCC1] |
| 23 | 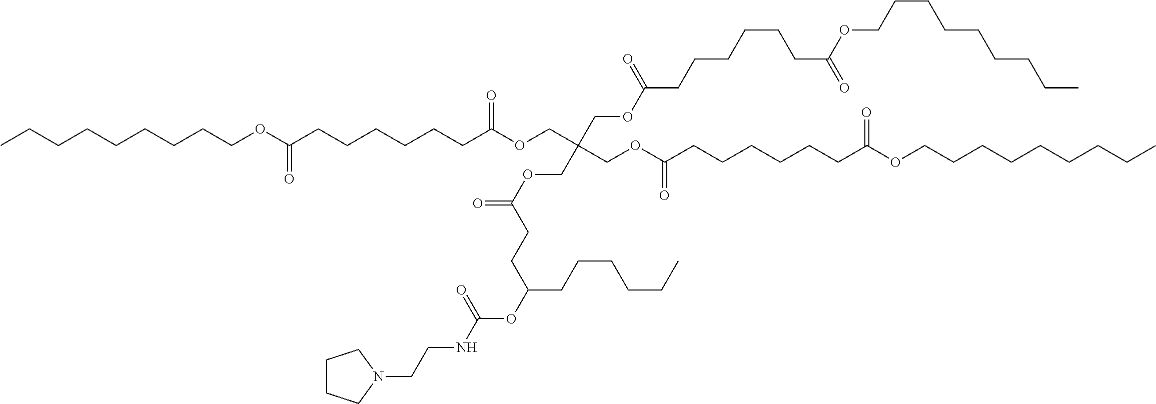 [SMILES: CCCCCCCCCOC(=O)CCCCCCC(=O)OCC(COC(=O)CCCCCCC(=O)OCCCCCCCCC)(COC(=O)CCCCCCC(=O)OCCCCCCCCC)COC(=O)CCC(CCCCCC)OC(=O)NCCN1CCCC1] |
| 24 | 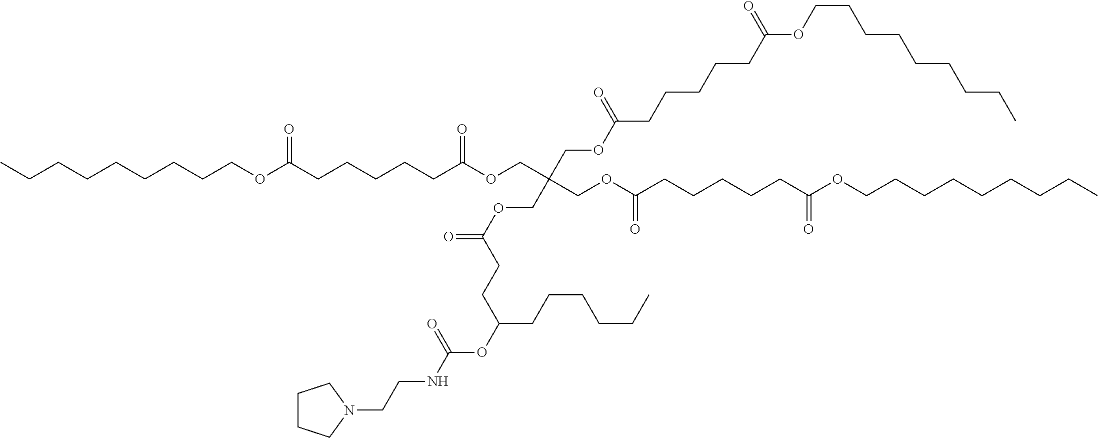 [SMILES: CCCCCCCCCOC(=O)CCCCCC(=O)OCC(COC(=O)CCCCCC(=O)OCCCCCCCCC)(COC(=O)CCCCCC(=O)OCCCCCCCCC)COC(=O)CCC(CCCCCC)OC(=O)NCCN1CCCC1] |
| 25 | 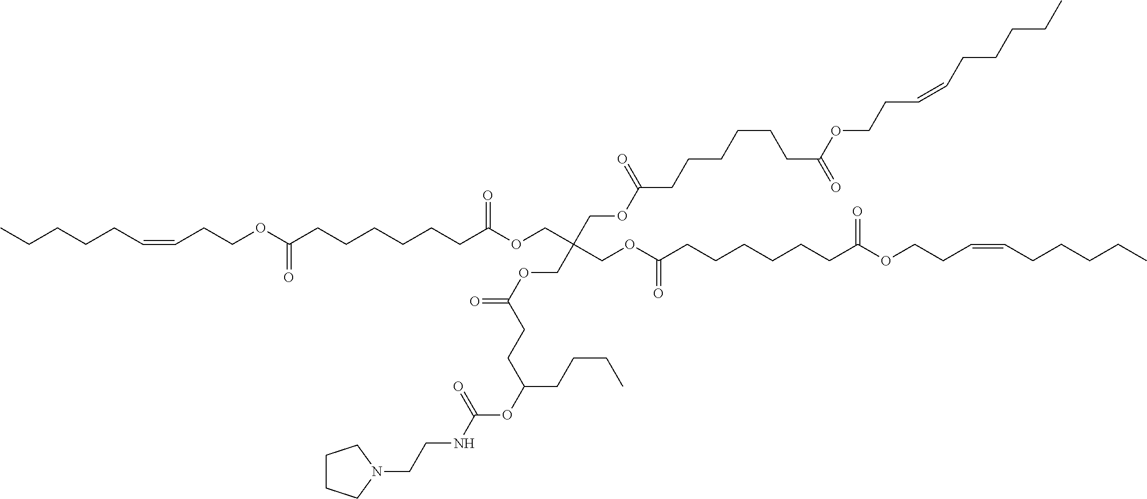 [SMILES: CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCC)OC(=O)NCCN1CCCC1] |
| 26 | 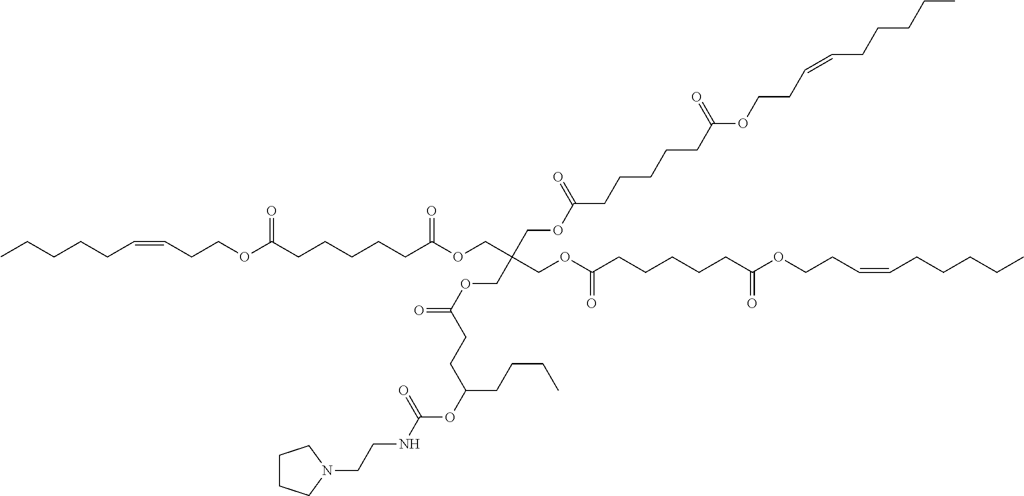 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCCC)OC(=O)NCCN1CCCC1] |
| 27 | 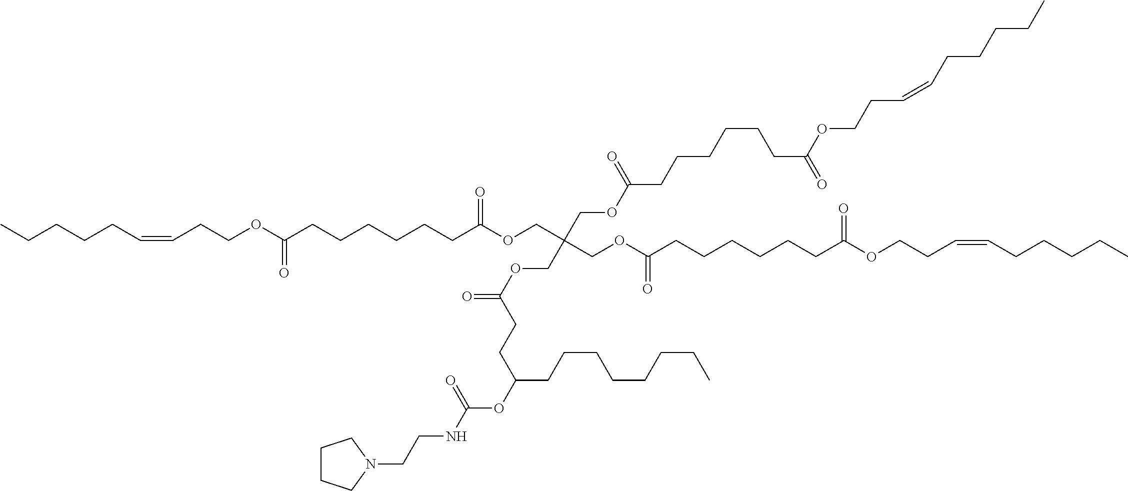 [SMILES: CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCCCCCC)OC(=O)NCCN1CCCC1] |
| 28 | 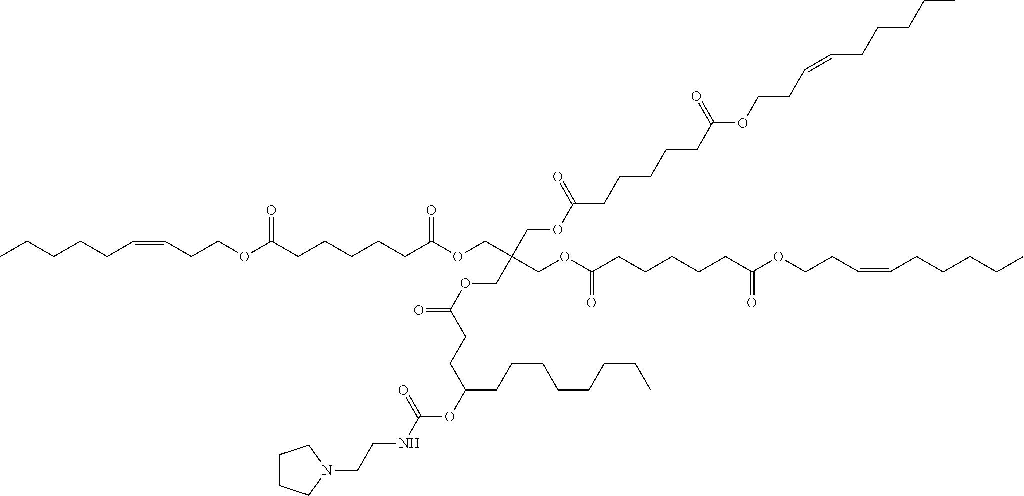 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCCCCCCC)OC(=O)NCCN1CCCC1] |
| 29 | 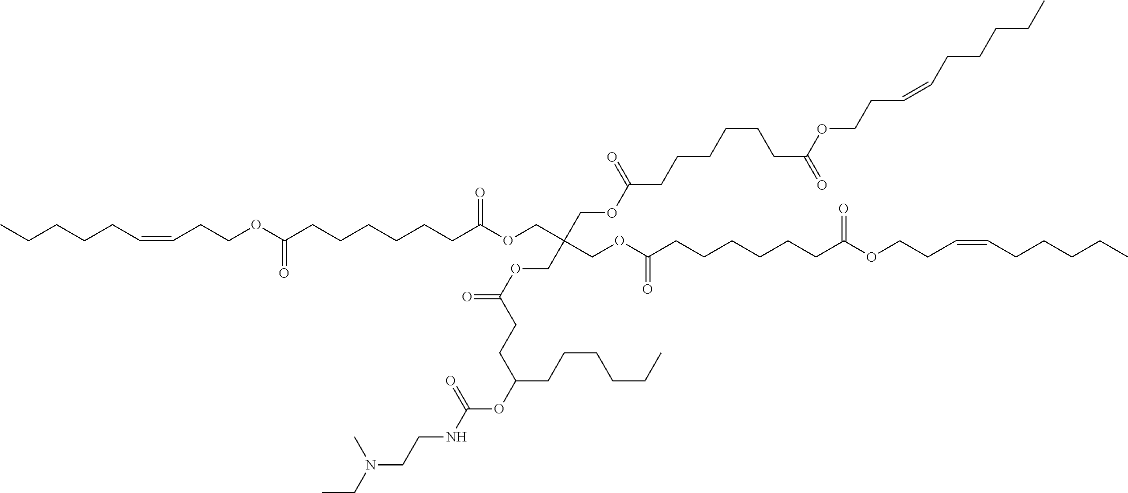 [SMILES: CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCCCC)OC(=O)NCCN(C)CC] |
| 30 | 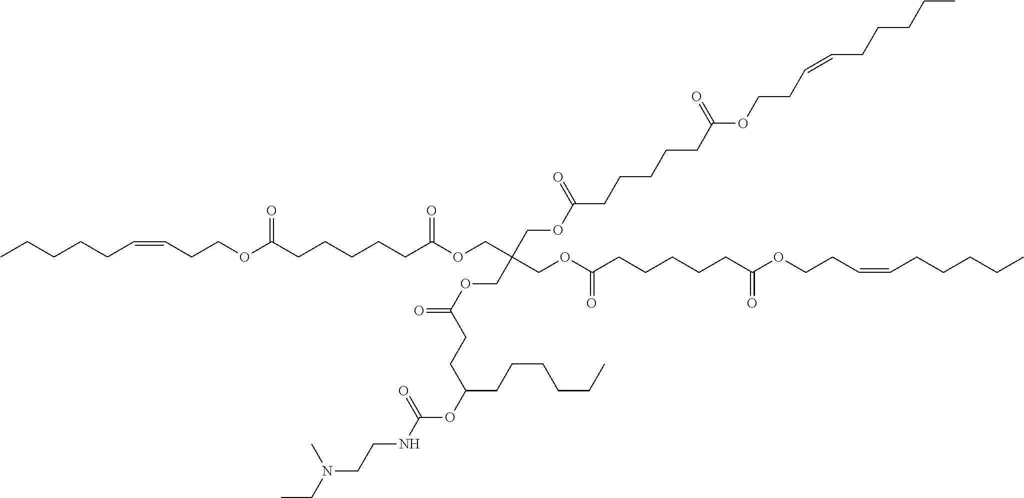 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCCCCC)OC(=O)NCCN(C)CC] |
| 31 | 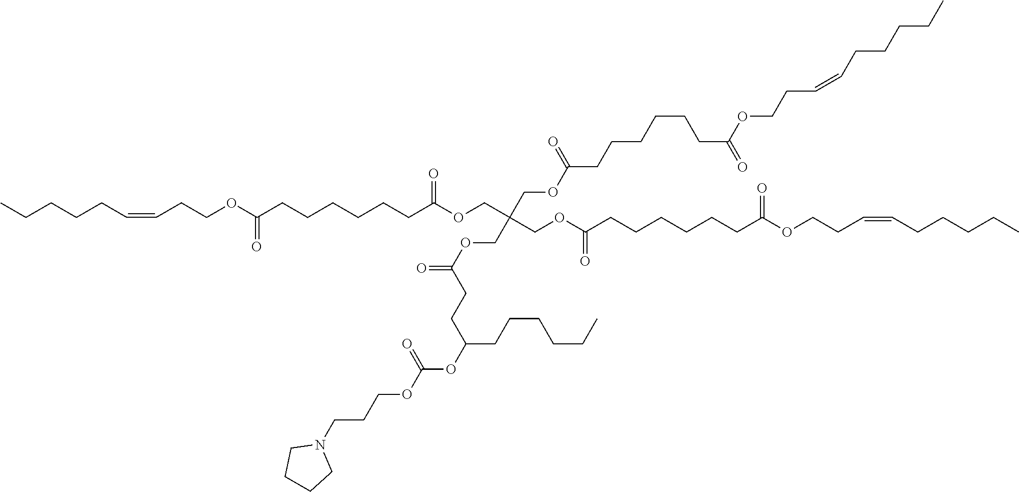 [SMILES: CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCCCC)OC(=O)OCCCN1CCCC1] |
| 32 | 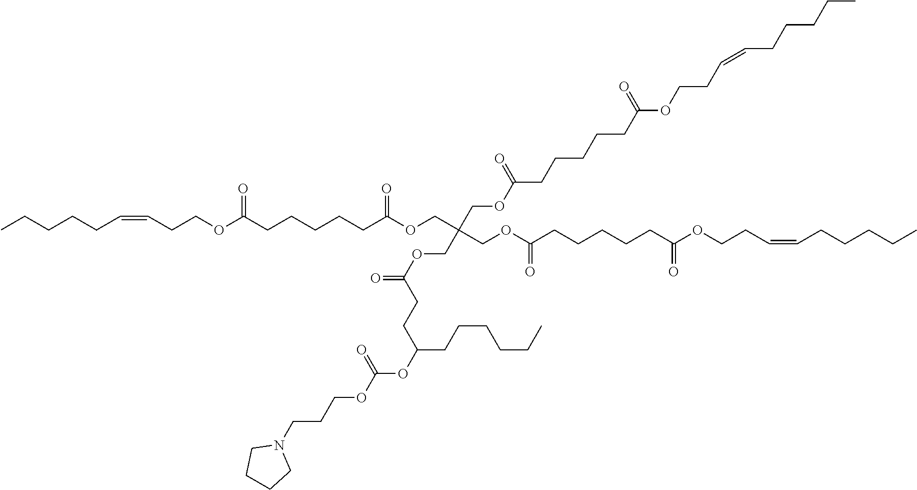 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCCCCC)OC(=O)OCCCN1CCCC1] |
| 33 | 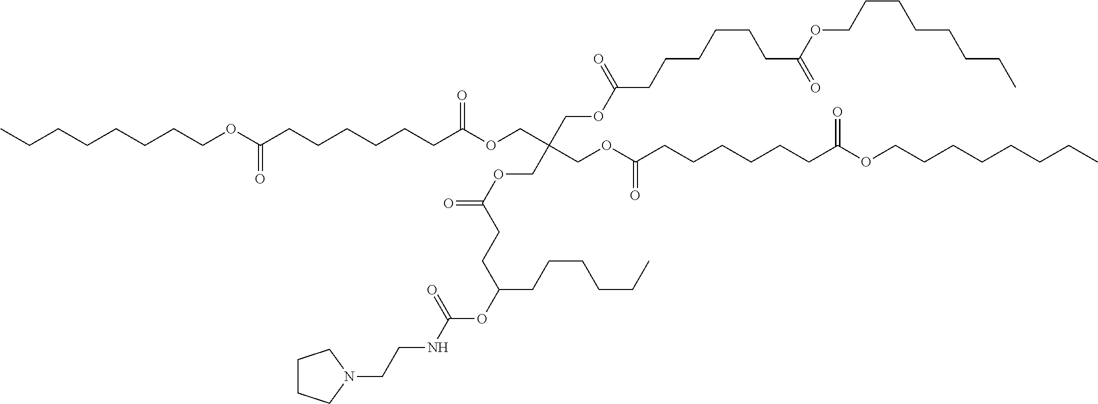 [SMILES: CCCCCCCCOC(=O)CCCCCCC(=O)OCC(COC(=O)CCCCCCC(=O)OCCCCCCCC)(COC(=O)CCCCCCC(=O)OCCCCCCCC)COC(=O)CCC(CCCCCC)OC(=O)NCCN1CCCC1] |
| 34 | 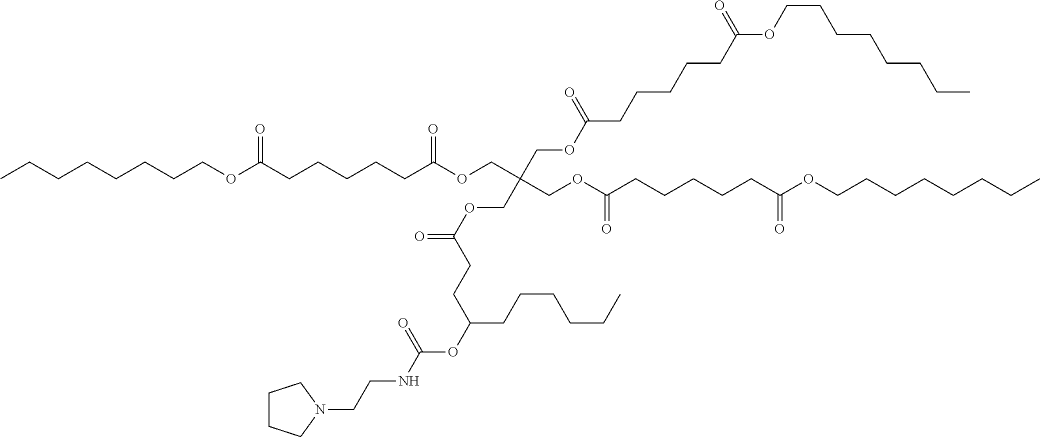 [SMILES: CCCCCCCCOC(=O)CCCCCC(=O)OCC(COC(=O)CCCCCC(=O)OCCCCCCCC)(COC(=O)CCCCCC(=O)OCCCCC(CC)CCC)COC(=O)CCC(CCCCCC)OC(=O)NCCN1CCCC1] |
| 35 | 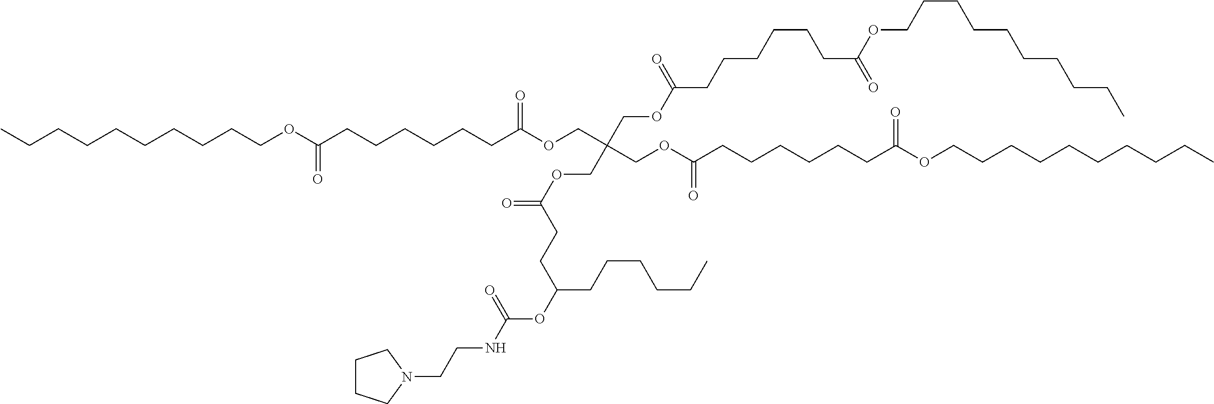 [SMILES: CCCCCCCCCCOC(=O)CCCCCCC(=O)OCC(COC(=O)CCCCCCC(=O)OCCCCCCCCCC)(COC(=O)CCCCCCC(=O)OCCCCCCCCCC)COC(=O)CCC(CCCCCC)OC(=O)NCCN1CCCC1] |
| 36 | 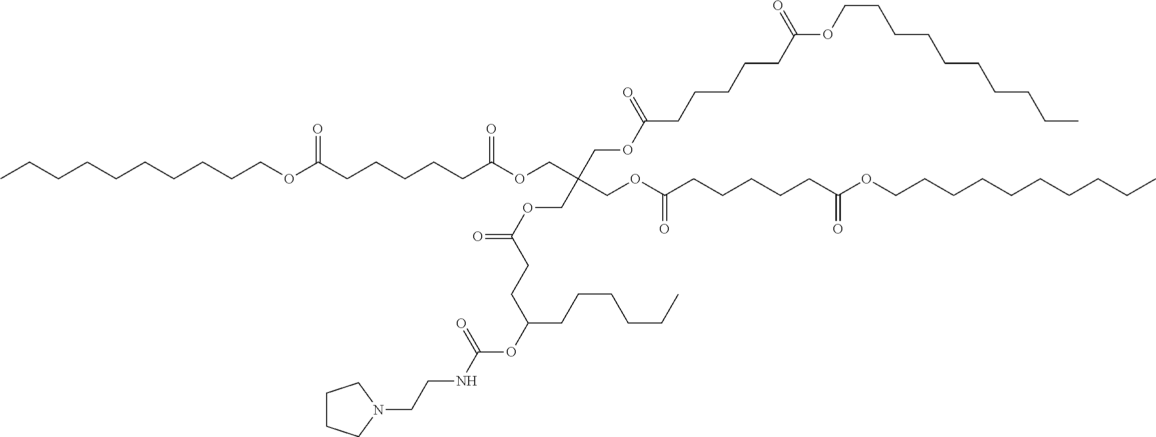 [SMILES: CCCCCCCCCCOC(=O)CCCCCC(=O)OCC(COC(=O)CCCCCC(=O)OCCCCCCCCCC)(COC(=O)CCCCCC(=O)OCCCCCCCCCC)COC(=O)CCC(CCCCCC)OC(=O)NCCN1CCCC1] |
| 37 | 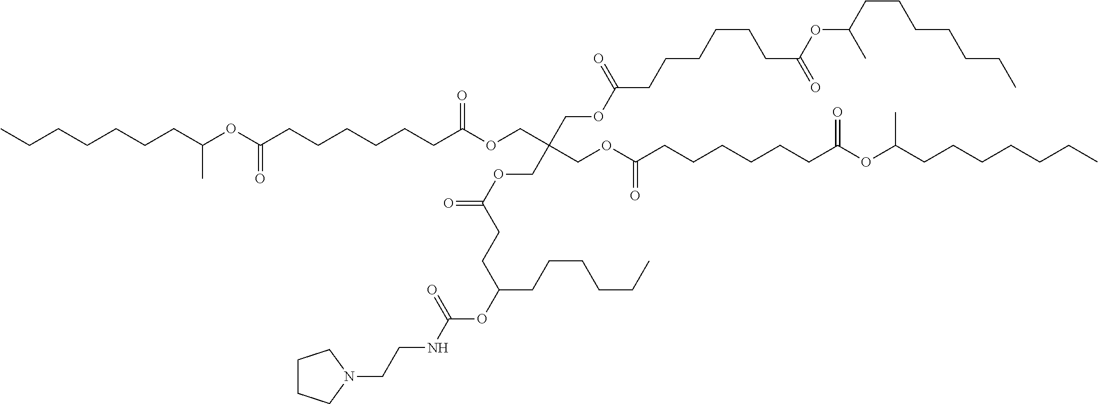 [SMILES: CCCCCCCC(C)OC(=O)CCCCCCC(=O)OCC(COC(=O)CCCCCCC(=O)OC(C)CCCCCCC)(COC(=O)CCCCCCC(=O)OC(C)CCCCCCC)COC(=O)CCC(CCCCCC)OC(=O)NCCN1CCCC1] |
| 38 | 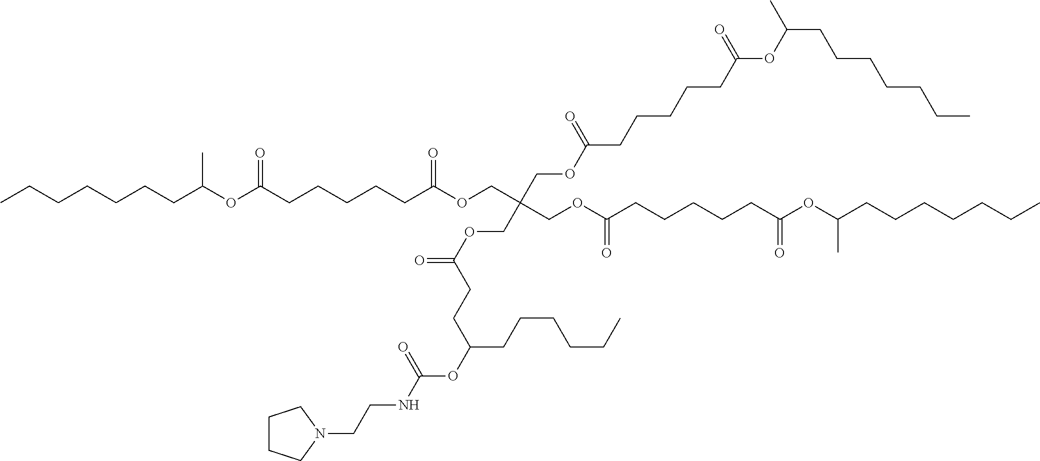 [SMILES: CCCCCCCC(C)OC(=O)CCCCCC(=O)OCC(COC(=O)CCCCCC(=O)OC(C)CCCCCCC)(COC(=O)CCCCCC(=O)OC(C)CCCCCCC)COC(=O)CCC(CCCCCC)OC(=O)NCCN1CCCC1] |
| 39 | 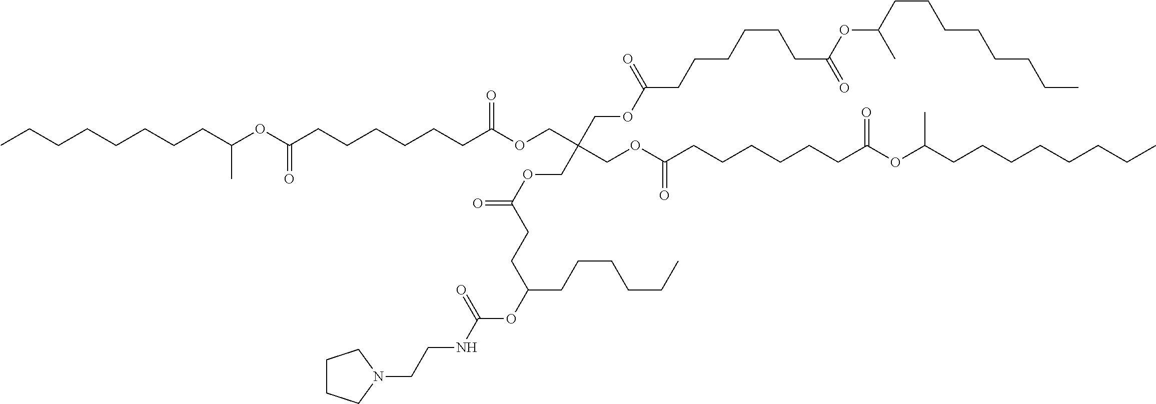 [SMILES: CCCCCCCCC(C)OC(=O)CCCCCCC(=O)OCC(COC(=O)CCCCCCC(=O)OC(C)CCCCCCCC)(COC(=O)CCCCCCC(=O)OC(C)CCCCCCCC)COC(=O)CCC(CCCCCC)OC(=O)NCCN1CCCC1] |
| 40 | 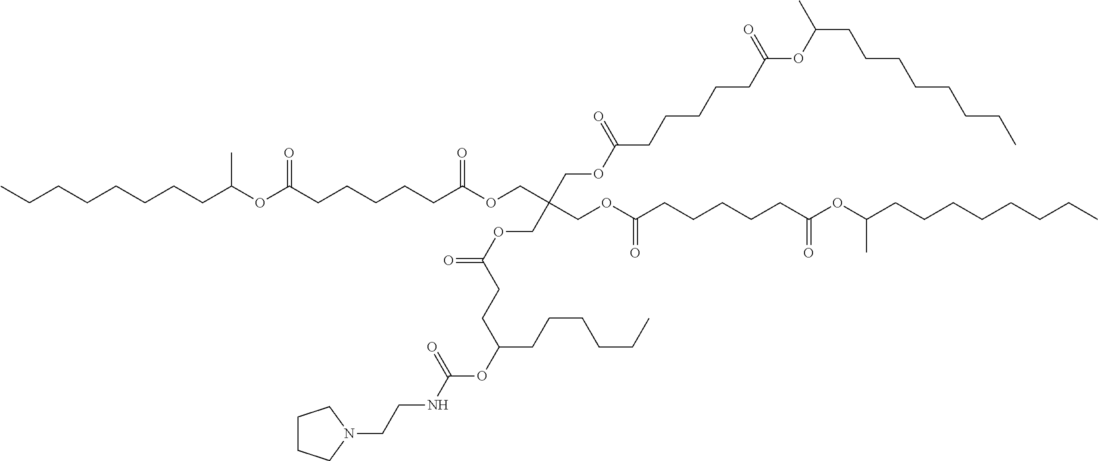 [SMILES: CCCCCCCCC(C)OC(=O)CCCCCC(=O)OCC(COC(=O)CCCCCC(=O)OC(C)CCCCCCCC)(COC(=O)CCCCCC(=O)OC(C)CCCCCCCC)COC(=O)CCC(CCCCCC)OC(=O)NCCN1CCCC1] |
| 41 | 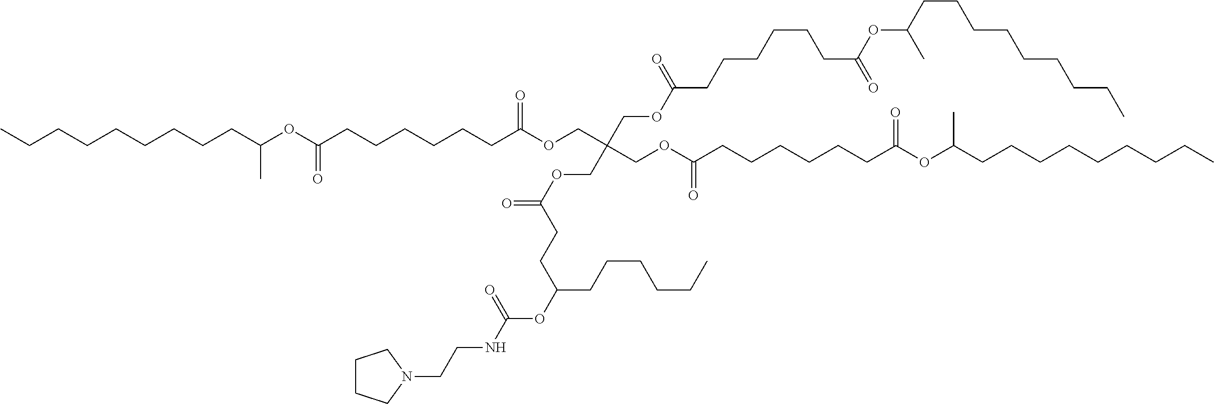 [SMILES: CCCCCCCCCC(C)OC(=O)CCCCCCC(=O)OCC(COC(=O)CCCCCCC(=O)OC(C)CCCCCCCCC)(COC(=O)CCCCCCC(=O)OC(C)CCCCCCCCC)COC(=O)CCC(CCCCCC)OC(=O)NCCN1CCCC1] |
| 42 | 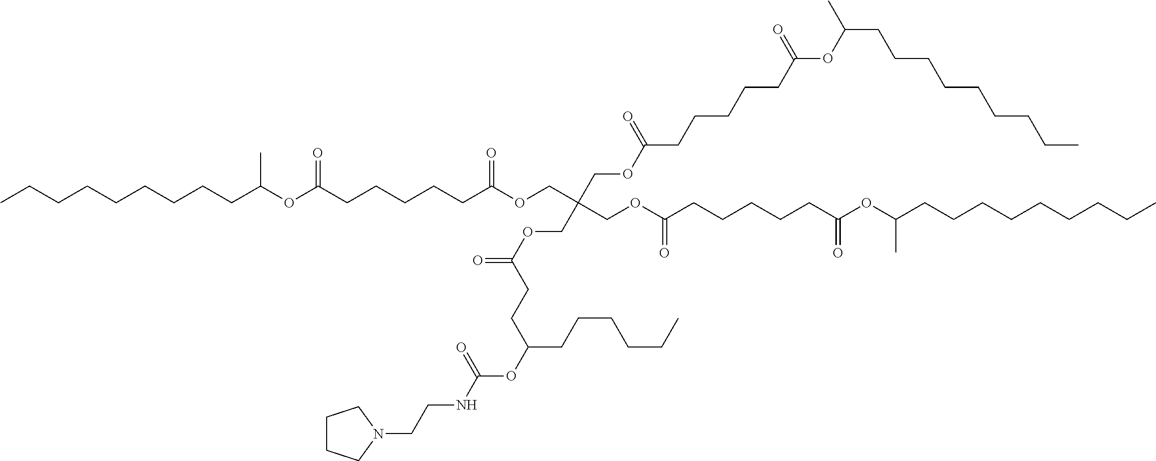 [SMILES: CCCCCCCCCC(C)OC(=O)CCCCCC(=O)OCC(COC(=O)CCCCCC(=O)OC(C)CCCCCCCCC)(COC(=O)CCCCCC(=O)OC(C)CCCCCCCCC)COC(=O)CCC(CCCCCC)OC(=O)NCCN1CCCC1] |
| 43 | 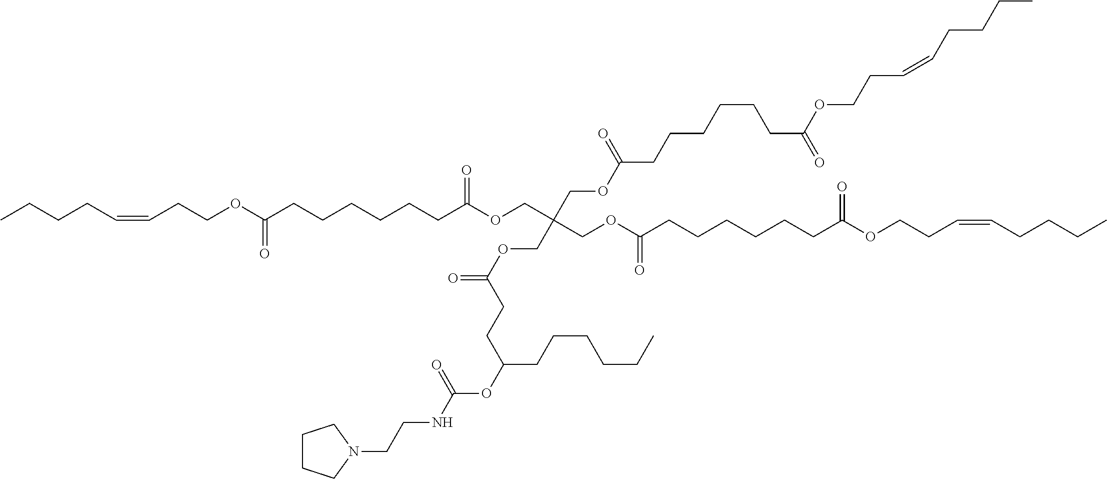 [SMILES: CCCC/C=C\CCOC(=O)CCCCCCC(=O)OCC(COC(=O)CCCCCCC(=O)OCC/C=C\CCCC)(COC(=O)CCCCCCC(=O)OCC/C=C\CCCC)COC(=O)CCC(CCCCCC)OC(=O)NCCN1CCCC1] |
| 44 | 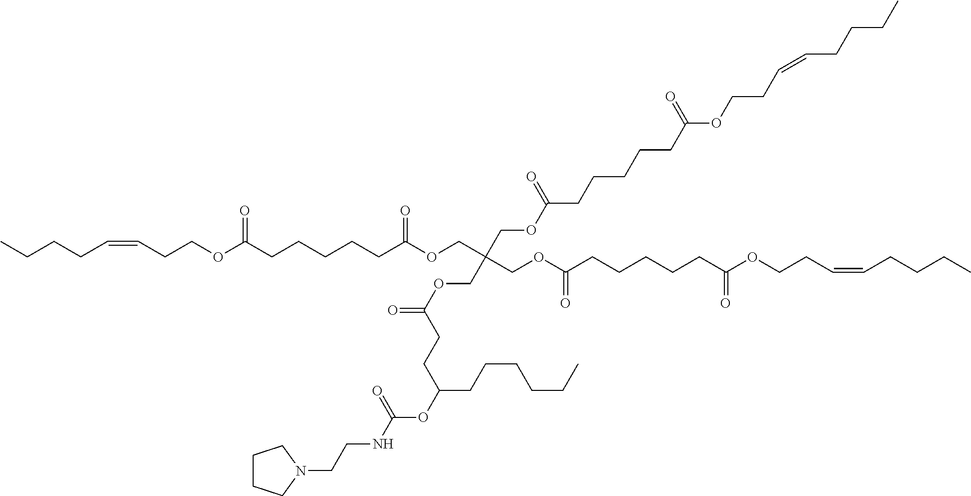 [SMILES: CCCC/C=C\CCOC(=O)CCCCCC(=O)OCC(COC(=O)CCCCCC(=O)OCC/C=C\CCCC)(COC(=O)CCCCCC(=O)OCC/C=C\CCCC)COC(=O)CCC(CCCCCC)OC(=O)NCCN1CCCC1] |
| 45 | 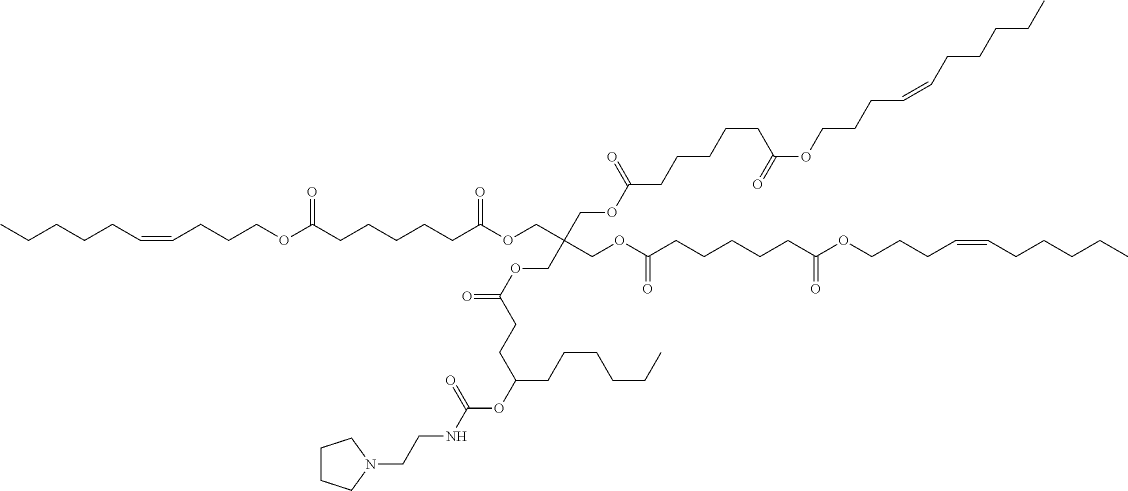 [SMILES: CCCCC/C=C\CCCOC(=O)CCCCCC(=O)OCC(COC(=O)CCCCCC(=O)OCCC/C=C\CCCCC)(COC(=O)CCCCCC(=O)OCCC/C=C\CCCCC)COC(=O)CCC(CCCCCC)OC(=O)NCCN1CCCC1] |
| 46 | 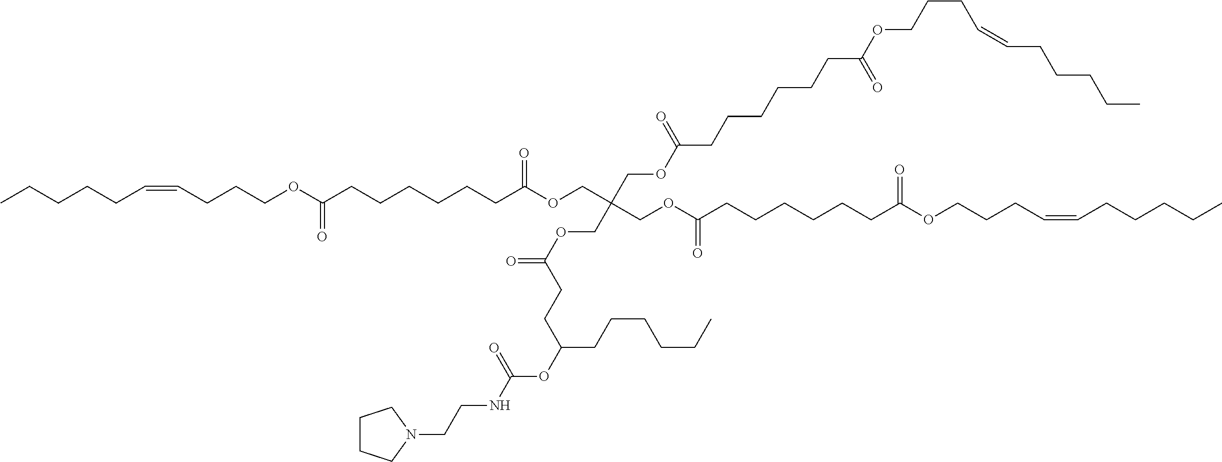 [SMILES: CCCCC/C=C\CCCOC(=O)CCCCCCC(=O)OCC(COC(=O)CCCCCCC(=O)OCCC/C=C\CCCCC)(COC(=O)CCCCCCC(=O)OCCC/C=C\CCCCC)COC(=O)CCC(CCCCCC)OC(=O)NCCN1CCCC1] |
| 47 | 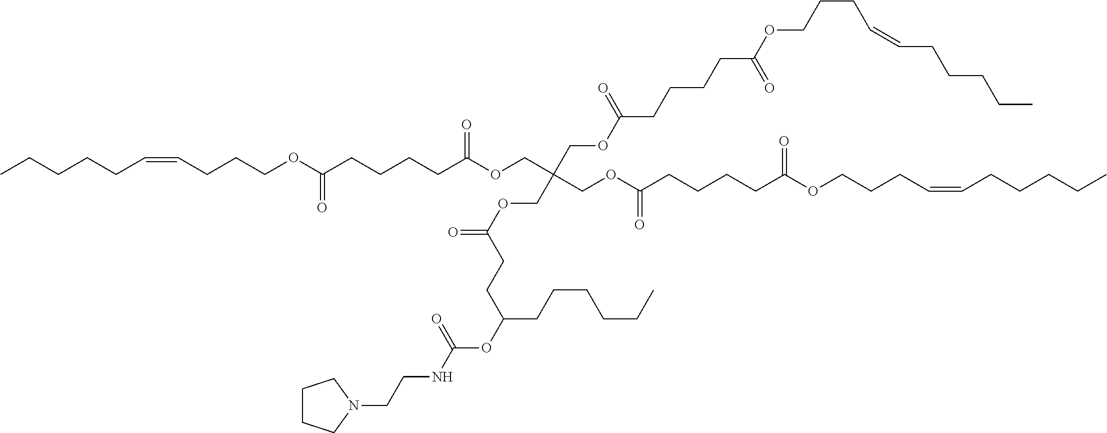 [SMILES: CCCCC/C=C\CCCOC(=O)CCCCC(=O)OCC(COC(=O)CCCCC(=O)OCCC/C=C\CCCCC)(COC(=O)CCCCC(=O)OCCC/C=C\CCCCC)COC(=O)CCC(CCCCCC)OC(=O)NCCN1CCCC1] |
| 48 | 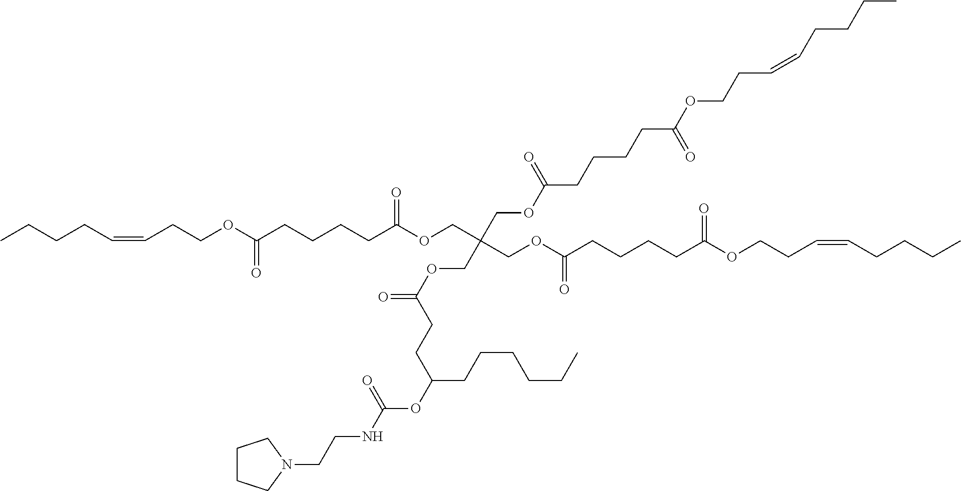 [SMILES: CCCC/C=C\CCOC(=O)CCCCC(=O)OCC(COC(=O)CCCCC(=O)OCC/C=C\CCCC)(COC(=O)CCCCC(=O)OCC/C=C\CCCC)COC(=O)CCC(CCCCCC)OC(=O)NCCN1CCCC1] |
| 49 | 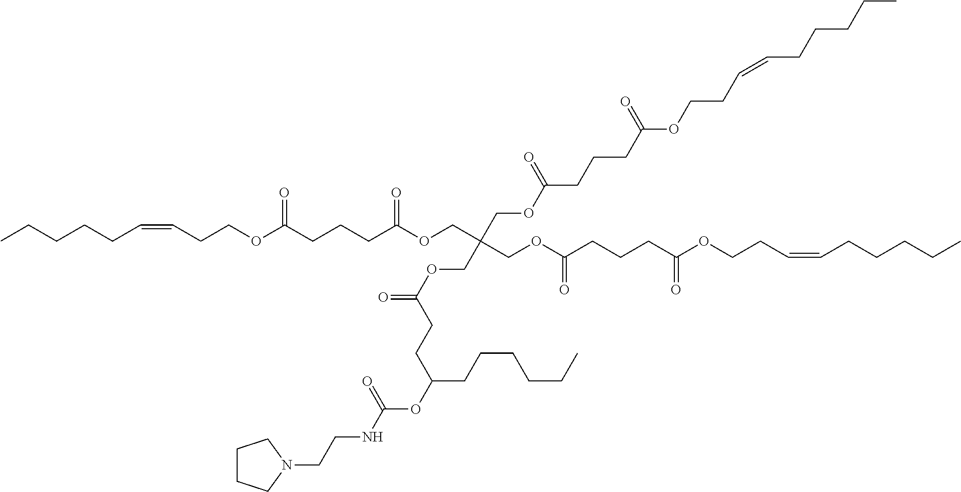 [SMILES: CCCCC/C=C\CCOC(=O)CCCC(=O)OCC(COC(=O)CCCC(=O)OCC/C=C\CCCCC)(COC(=O)CCCC(=O)OCC/C=C\CCCCC)COC(=O)CCC(CCCCCC)OC(=O)NCCN1CCCC1] |
| 50 | 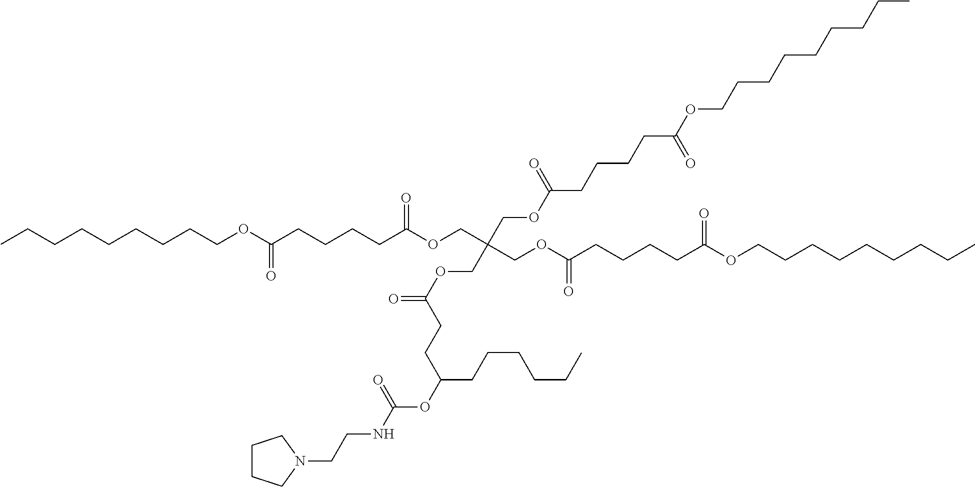 [SMILES: CCCCCCCCCOC(=O)CCCCC(=O)OCC(COC(=O)CCCCC(=O)OCCCCCCCCC)(COC(=O)CCCCC(=O)OCCCCCCCCC)COC(=O)CCC(CCCCCC)OC(=O)NCCN1CCCC1] |
| 51 | 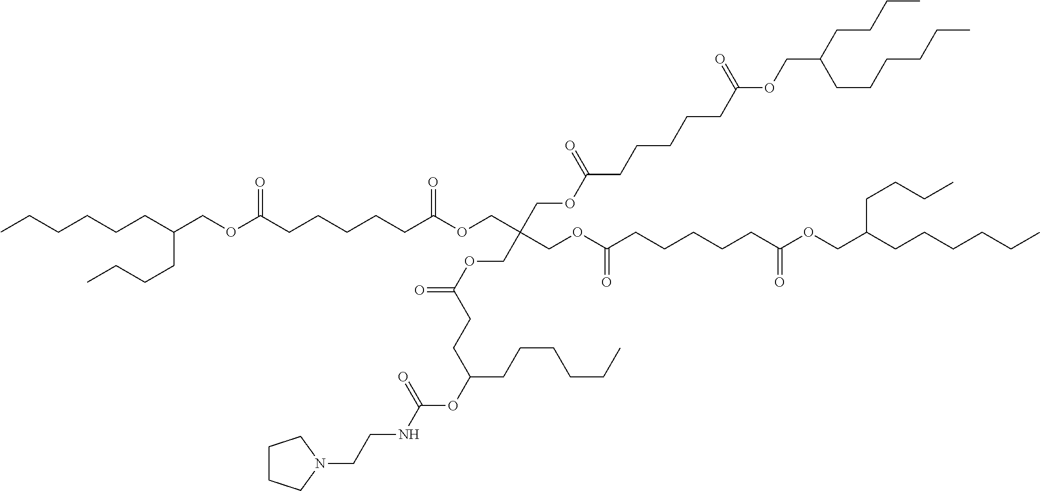 [SMILES: CCCCCCC(CCCC)COC(=O)CCCCCC(=O)OCC(COC(=O)CCCCCC(=O)OCC(CCCC)CCCCCC)(COC(=O)CCCCCC(=O)OCC(CCCC)CCCCCC)COC(=O)CCC(CCCCCC)OC(=O)NCCN1CCCC1] |
| 52 | 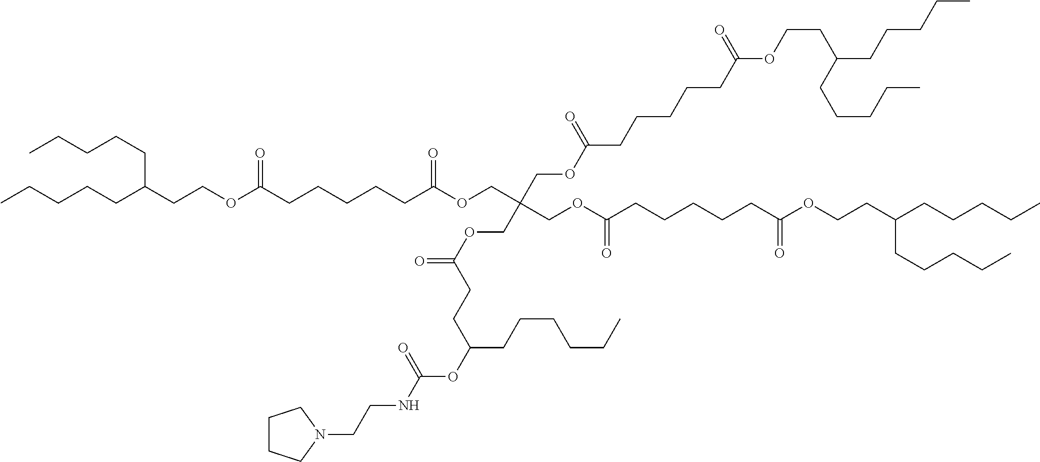 [SMILES: CCCCCCC(CCC(=O)OCC(COC(=O)CCCCCC(=O)OCCC(CCCCC)CCCCC)(COC(=O)CCCCCC(=O)OCCC(CCCCC)CCCCC)COC(=O)CCCCCC(=O)OCCC(CCCCC)CCCCC)OC(=O)NCCN1CCCC1] |
| 53 | 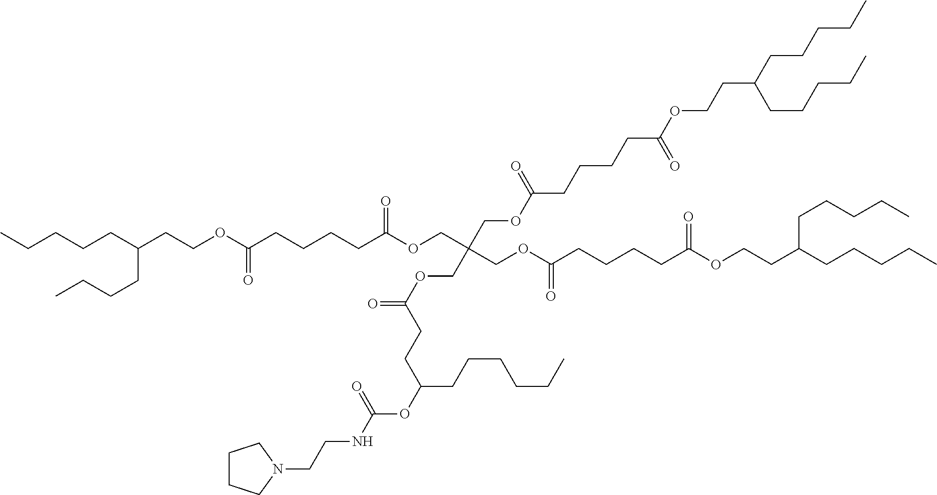 [SMILES: CCCCCCC(CCC(=O)OCC(COC(=O)CCCCC(=O)OCCC(CCCC)CCCCC)(COC(=O)CCCCC(=O)OCCC(CCCCC)CCCCC)COC(=O)CCCCC(=O)OCCC(CCCCC)CCCCC)OC(=O)NCCN1CCCC1] |
| 54 | 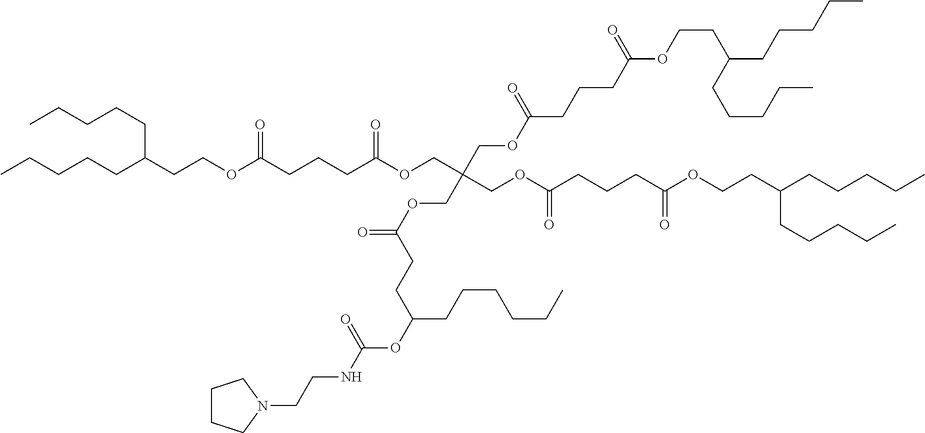 [SMILES: CCCCCCC(CCC(=O)OCC(COC(=O)CCCC(=O)OCCC(CCCCC)CCCCC)(COC(=O)CCCC(=O)OCCC(CCCCC)CCCCC)COC(=O)CCCC(=O)OCCC(CCCCC)CCCCC)OC(=O)NCCN1CCCC1] |
| 55 | 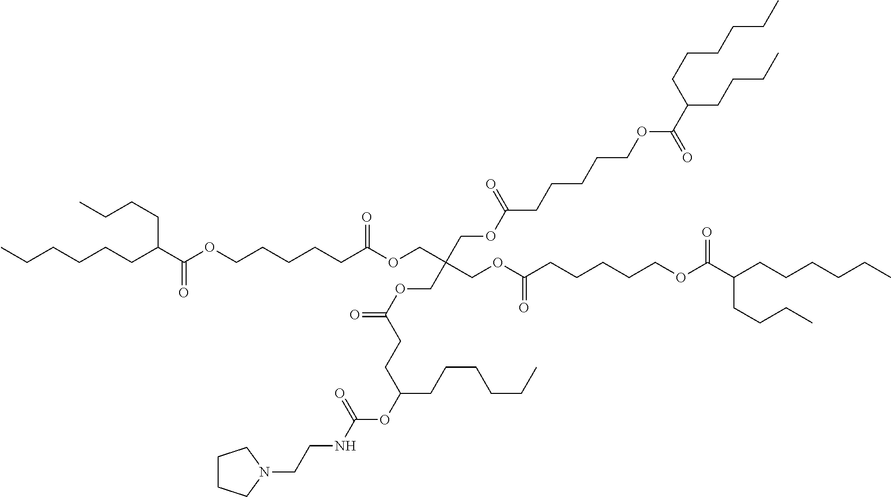 [SMILES: CCCCCCC(CCC(=O)OCC(COC(=O)CCCCCOC(=O)C(CCCC)CCCCCC)(COC(=O)CCCCCOC(=O)C(CCCC)CCCCCC)COC(=O)CCCCCOC(=O)C(CCCC)CCCCCC)OC(=O)NCCN1CCCC1] |
| 56 | 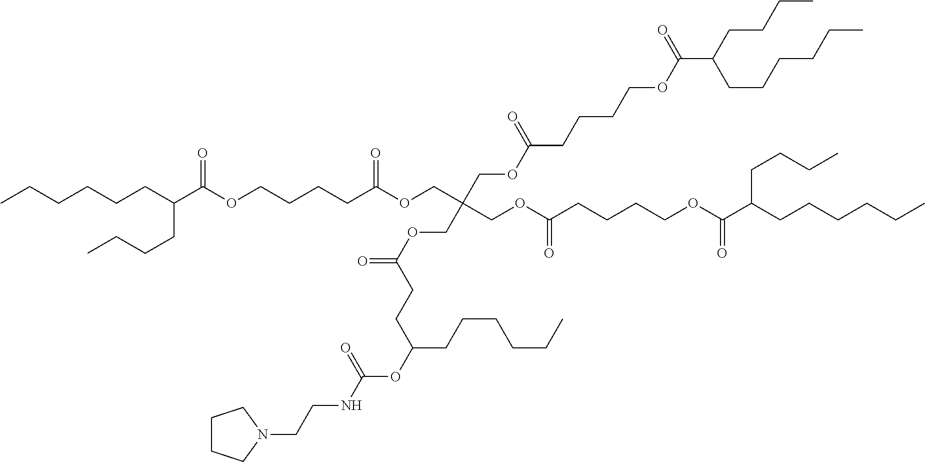 [SMILES: CCCCCCC(CCC(=O)OCC(COC(=O)CCCCOC(=O)C(CCCC)CCCCCC)(COC(=O)CCCCOC(=O)C(CCCC)CCCCCC)COC(=O)CCCCOC(=O)C(CCCC)CCCCCC)OC(=O)NCCN1CCCC1] |
| 57 | 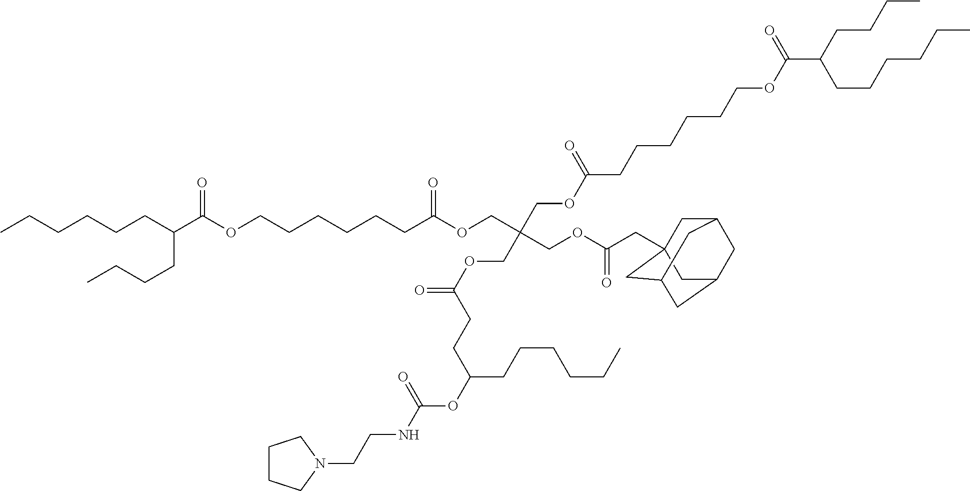 [SMILES: CCCCCCC(CCC(=O)OCC(COC(=O)CCCCCCOC(=O)C(CCCC)CCCCCC)(COC(=O)CCCCCCOC(=O)C(CCCC)CCCCCC)COC(=O)CC12CC3CC(CC(C3)C1)C2)OC(=O)NCCN1CCCC1] |
| 58 | 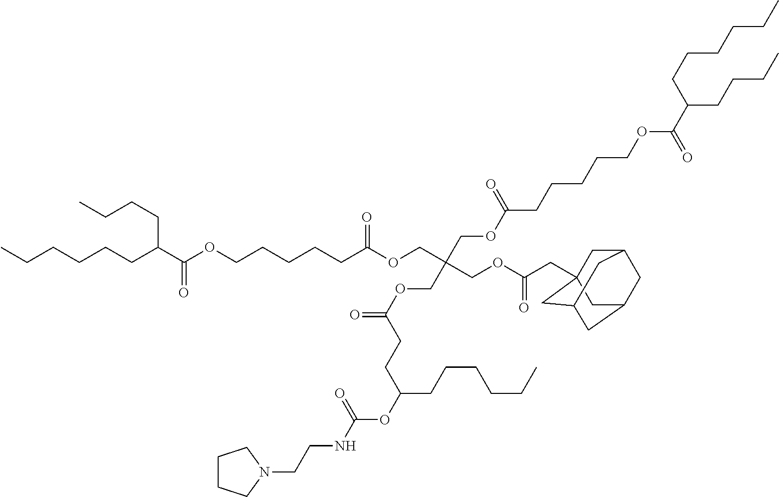 [SMILES: CCCCCCC(CCC(=O)OCC(COC(=O)CCCCCOC(=O)C(CCCC)CCCCCC)(COC(=O)CCCCCOC(=O)C(CCCC)CCCCCC)COC(=O)CC12CC3CC(CC(C3)C1)C2)OC(=O)NCCN1CCCC1] |
| 59 | 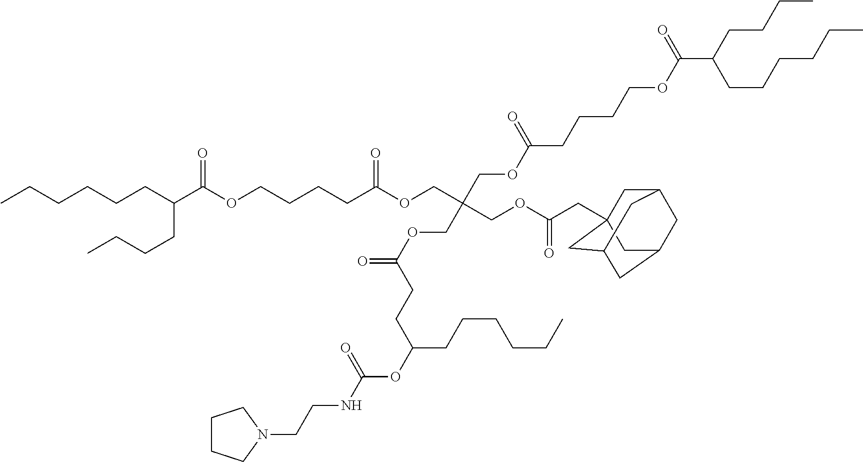 [SMILES: CCCCCCC(CCC(=O)OCC(COC(=O)CCCCOC(=O)C(CCCC)CCCCCC)(COC(=O)CCCCOC(=O)C(CCCC)CCCCCC)COC(=O)CC12CC3CC(CC(C3)C1)C2)OC(=O)NCCN1CCCC1] |
| 60 | 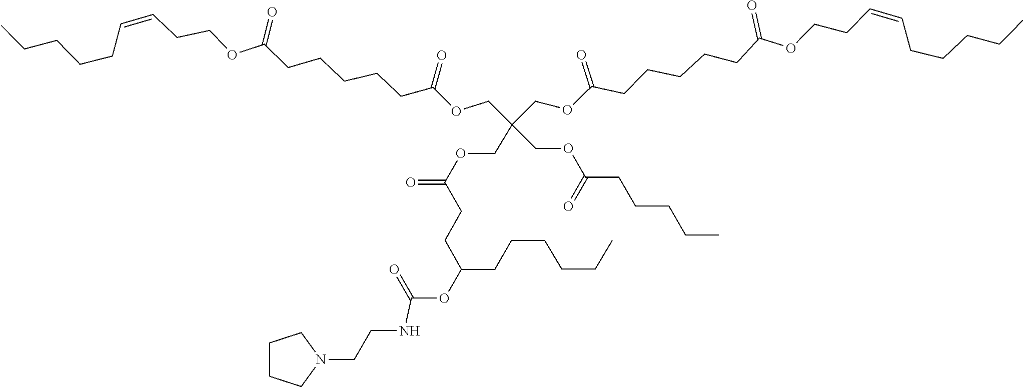 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCCCCC)OC(=O)NCCN1CCCC1] |
| 61 | 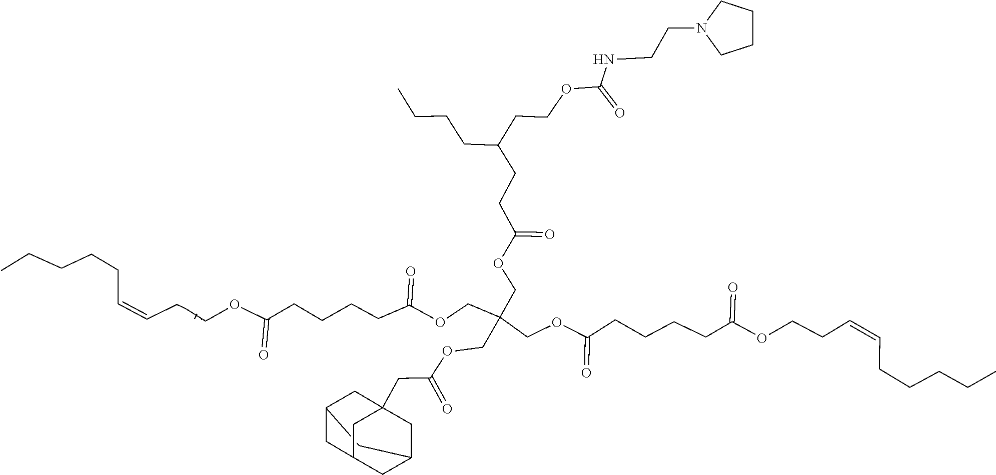 [SMILES: CCCCC/C=C\CCOC(=O)CCCCC(=O)OCC(COC(=O)CCCCC(=O)O[C@H](C)CC/C=C\CCCCC)(COC(=O)CCC(CCCCC)CCOC(=O)NCCN1CCCC1)COC(=O)CC12CC3CC(CC(C3)C1)C2] |
| 62 | 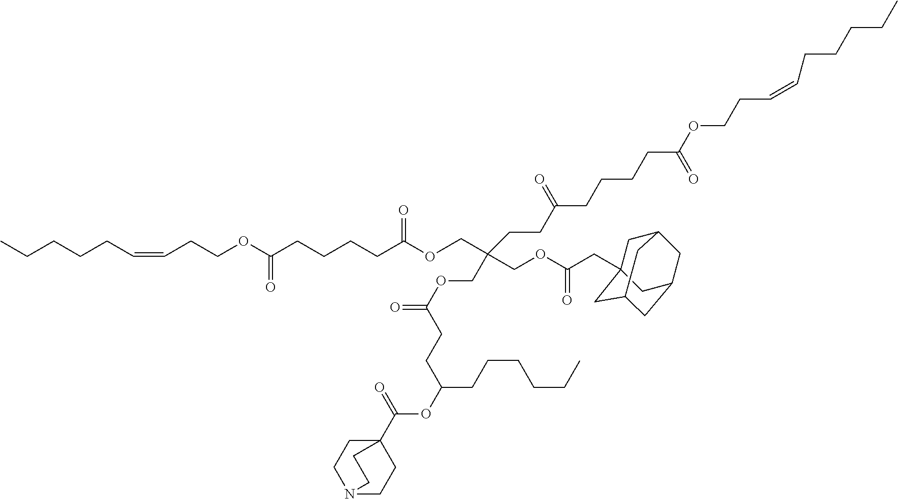 [SMILES: CCCCC/C=C\CCOC(=O)CCCCC(=O)CCC(COC(=O)CCCCC(=O)OCC/C=C\CCCCC)(COC(=O)CCC(CCCCCC)OC(=O)C12CCN(CC1)CC2)COC(=O)CC12CC3CC(CC(C3)C1)C2] |
| 63 | 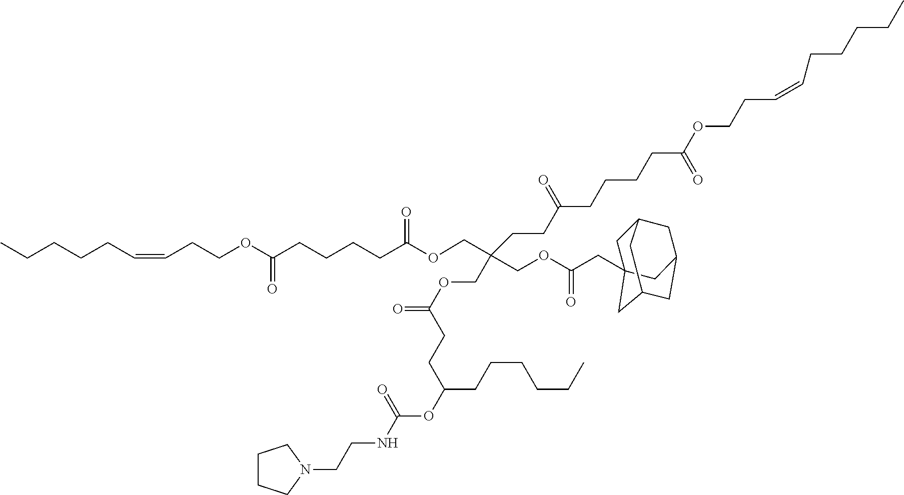 [SMILES: CCCCC/C=C\CCOC(=O)CCCCC(=O)CCC(COC(=O)CCCCC(=O)OCC/C=C\CCCCC)(COC(=O)CCC(CCCCCC)OC(=O)NCCN1CCCC1)COC(=O)CC12CC3CC(CC(C3)C1)C2] |
| 64 | 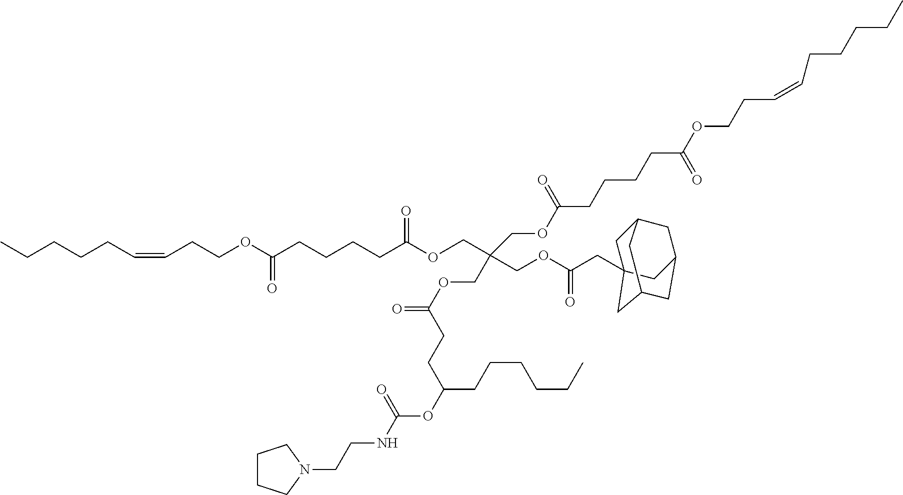 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC12CC3CC(CC(C3)C1)C2] |
| 65 | 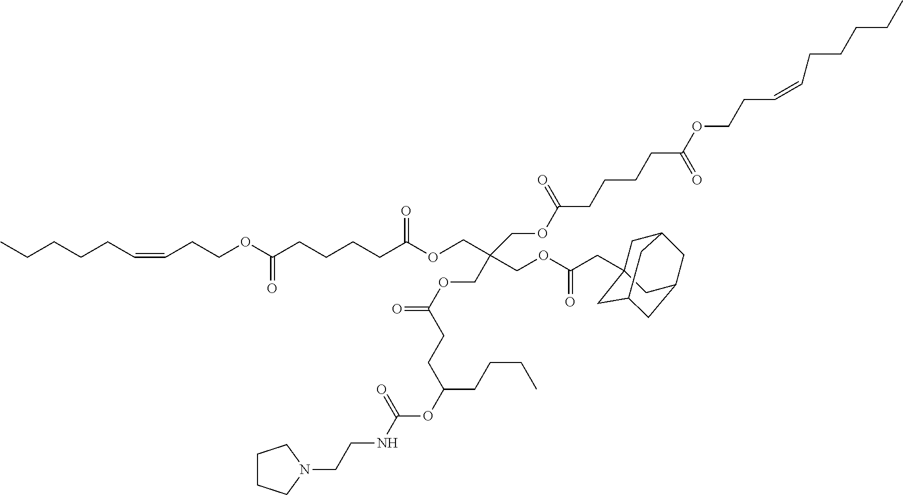 [SMILES: CCCCC/C=C\CCOC(=O)CCCCC(=O)OCC(COC(=O)CCCCC(=O)OCC/C=C\CCCCC)(COC(=O)CCC(CCCC)OC(=O)NCCN1CCCC1)COC(=O)CC12CC3CC(CC(C3)C1)C2] |
| 66 | 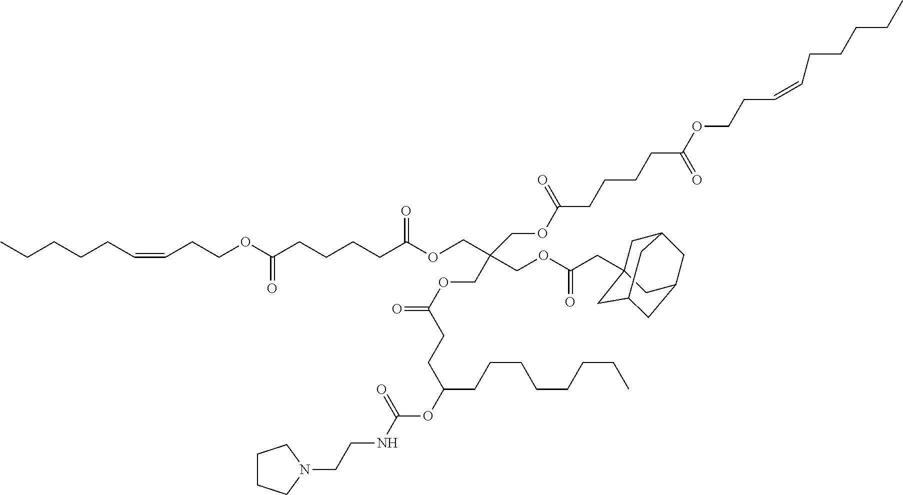 [SMILES: CCCCC/C=C\CCOC(=O)CCCCC(=O)OCC(COC(=O)CCCCC(=O)OCC/C=C\CCCCC)(COC(=O)CCC(CCCCCCCC)OC(=O)NCCN1CCCC1)COC(=O)CC12CC3CC(CC(C3)C1)C2] |
| 67 | 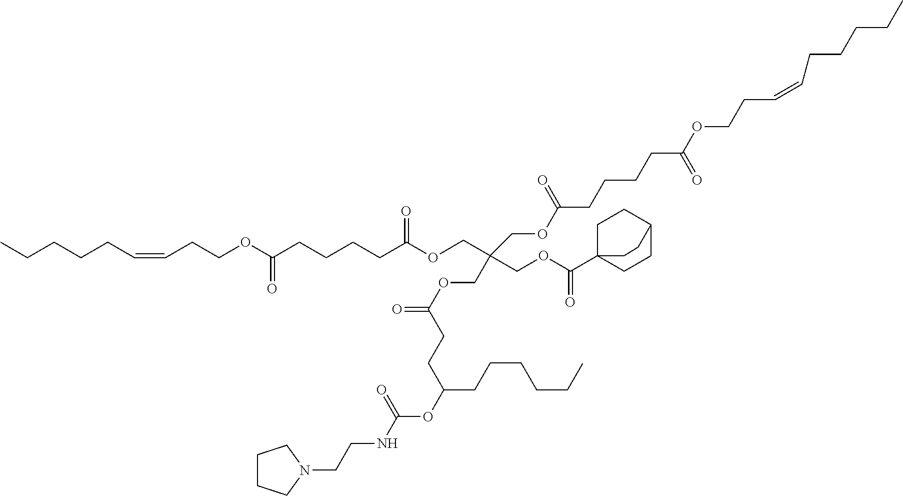 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(CC1)CC2] |
| 68 | 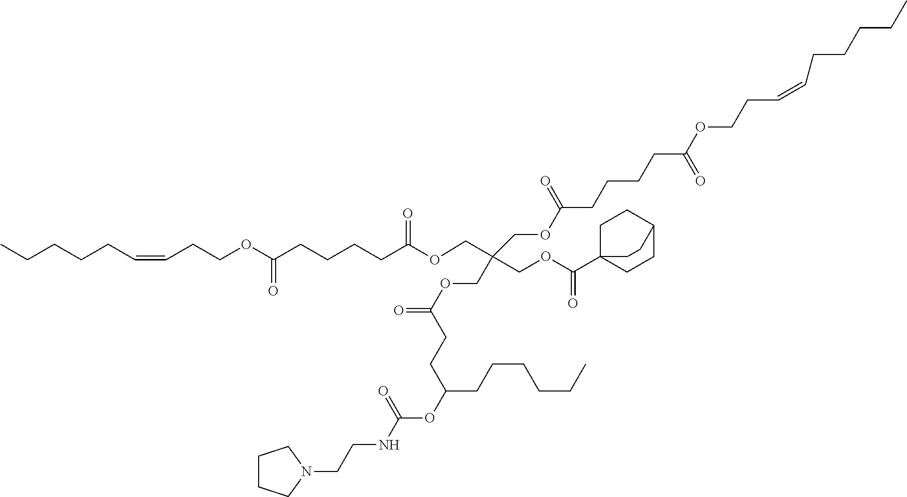 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(CC1)CC2] |
| 69 | 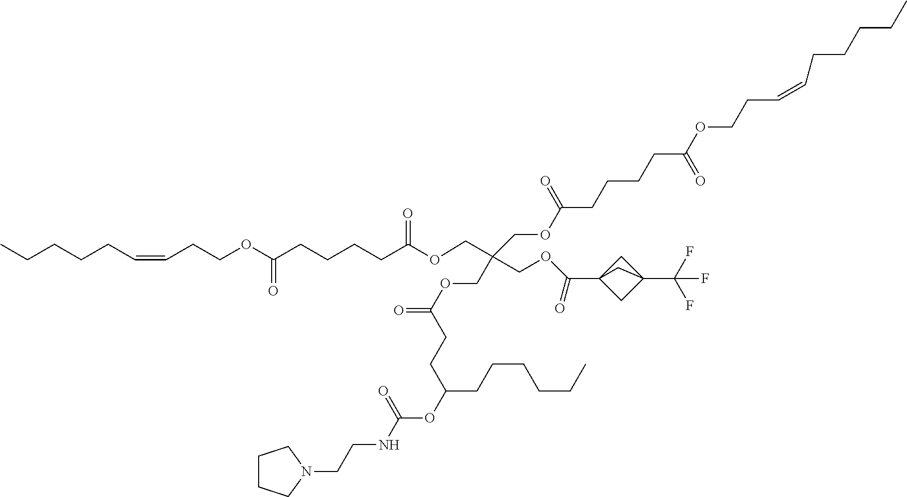 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C(F)(F)F)(C1)C2] |
| 70 | 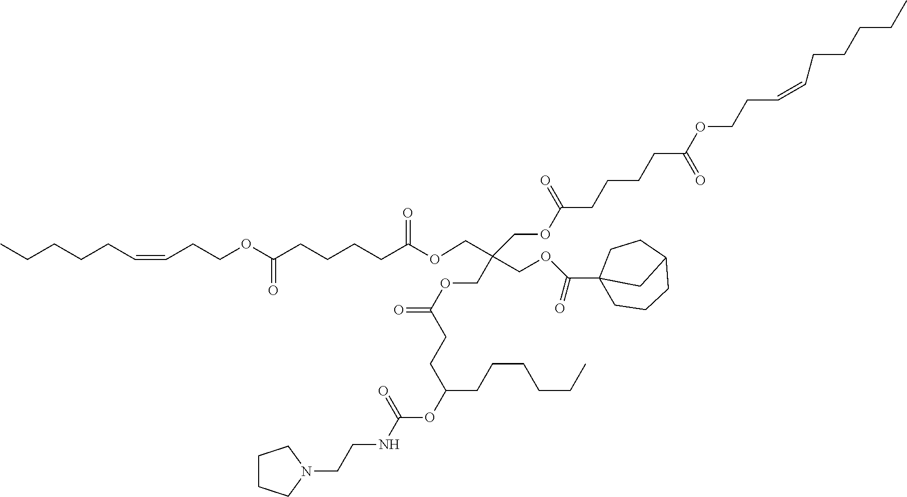 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCCC(CC1)C2] |
| 71 | 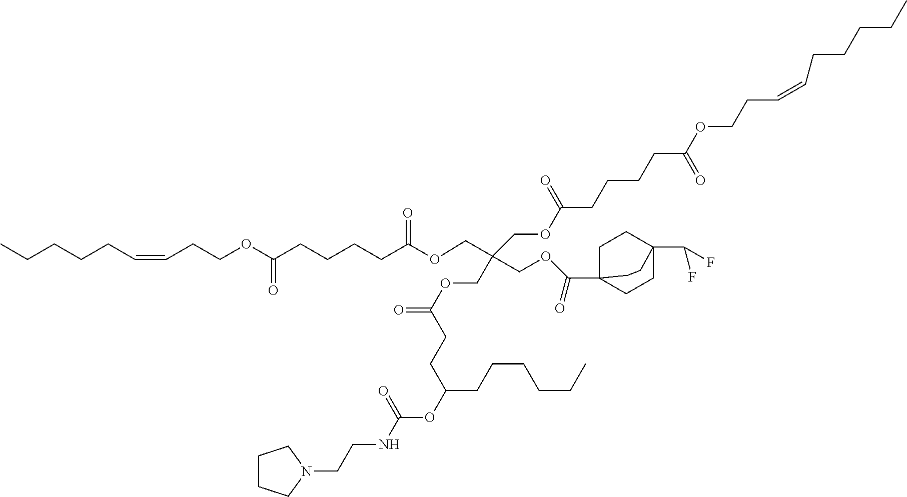 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(C(F)F)(CC1)CC2] |
| 72 | 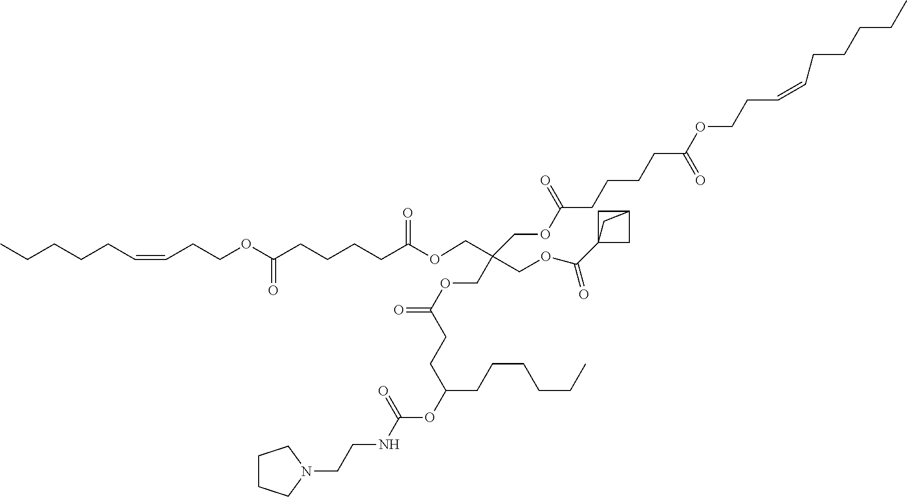 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C1)C2] |
| 73 | 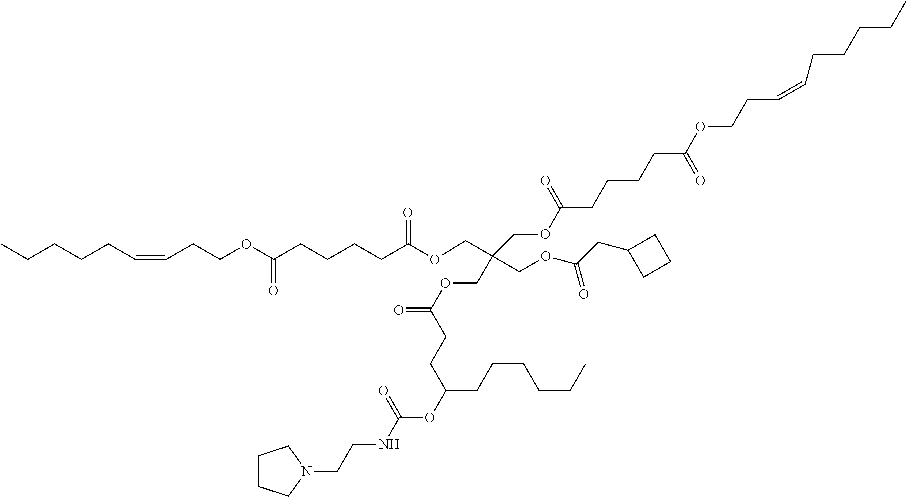 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC1CCC1] |
| 74 | 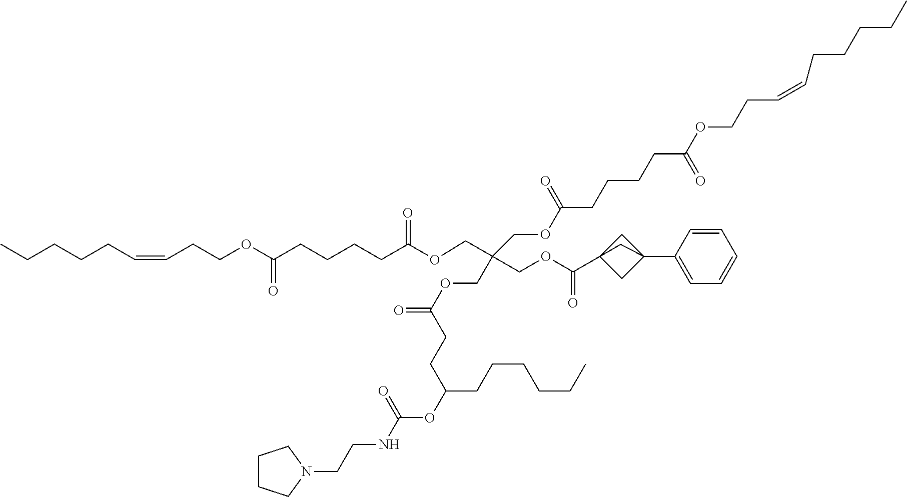 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(c3ccccc3)(C1)C2] |
| 75 | 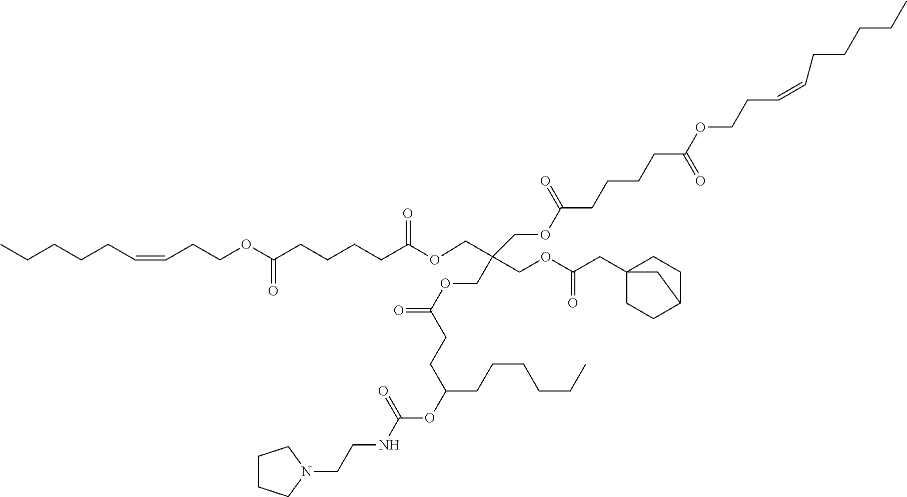 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC12CCC(CC1)C2] |
| 76 | 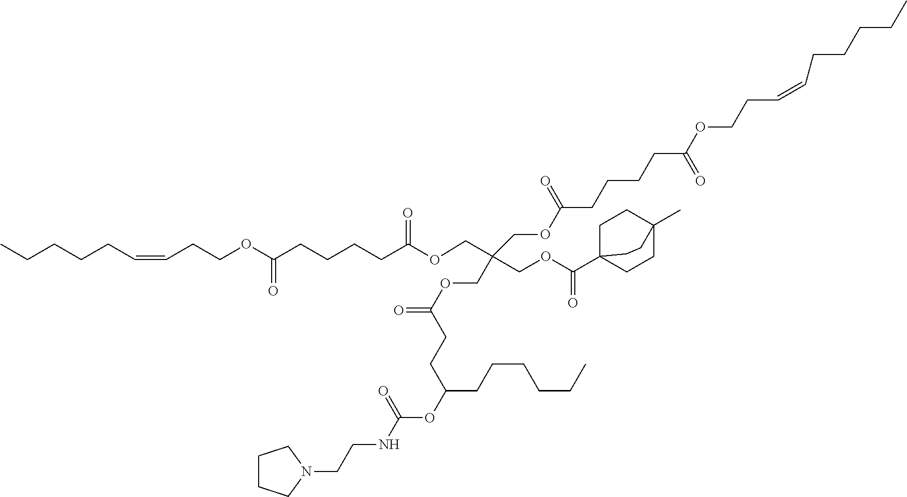 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(C)(CC1)CC2] |
| 77 | 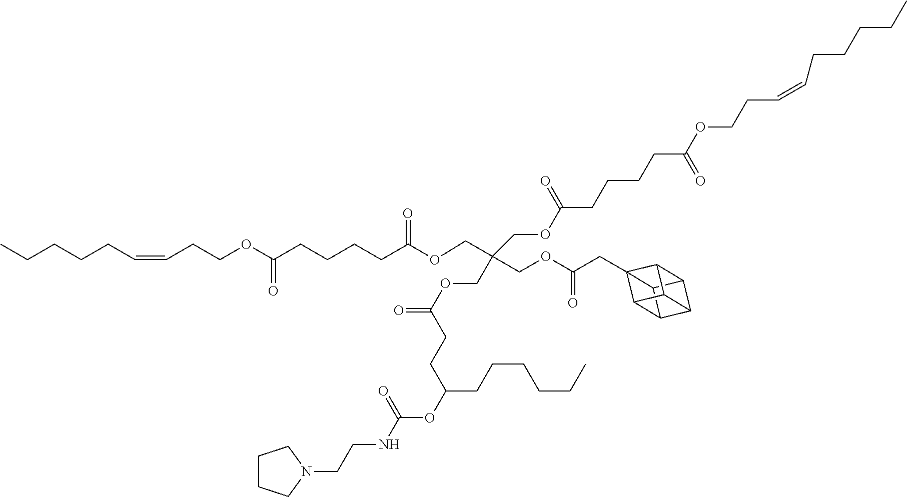 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC12C3C4C5C3C1C5C42] |
| 78 | 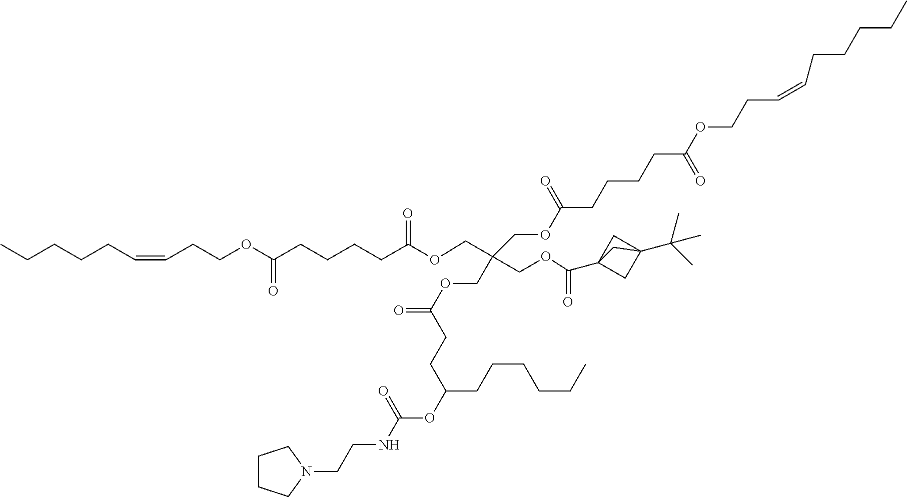 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C(C)(C)C)(C1)C2] |
| 79 | 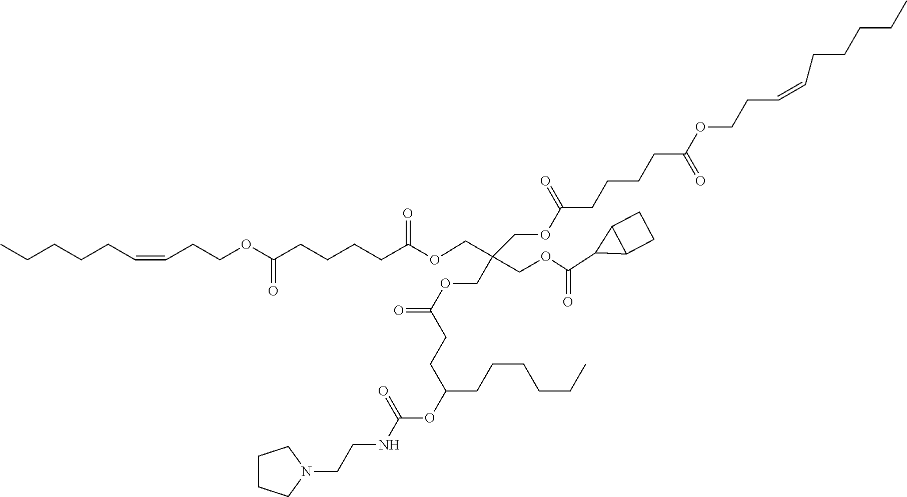 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1C2CCC21] |
| 80 | 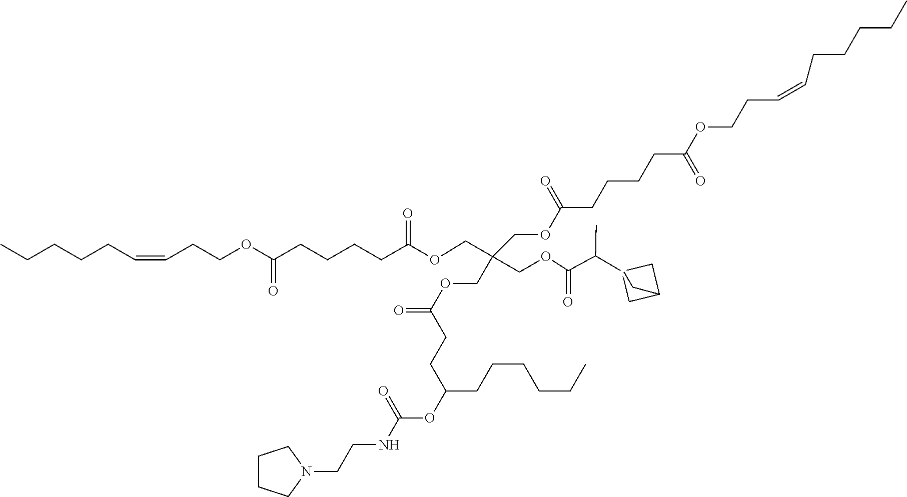 |
| 81 | 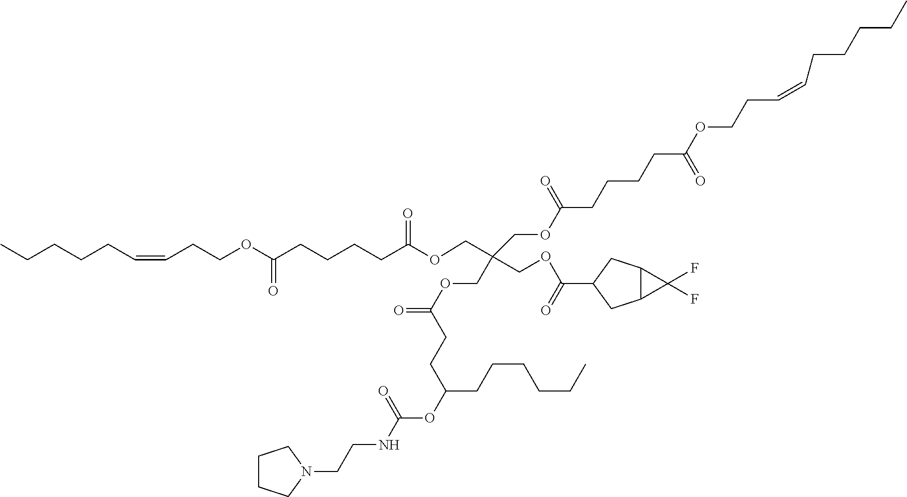 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CC2C(C1)C2(F)F] |
| 82 | 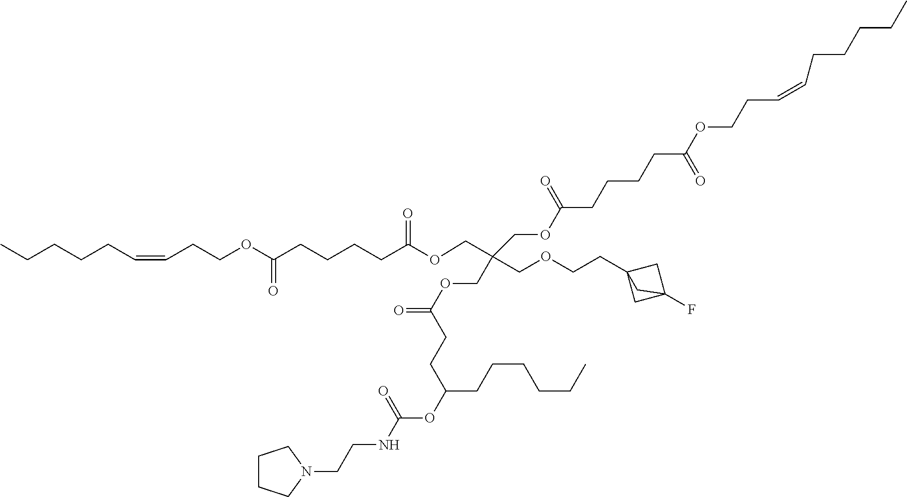 [SMILES: CCCCC/C=C\CCOC(=O)CCCCC(=O)OCC(COCCC12CC(F)(C1)C2)(COC(=O)CCCCC(=O)OCC/C=C\CCCCC)COC(=O)CCC(CCCCCC)OC(=O)NCCN1CCCC1] |
| 83 | 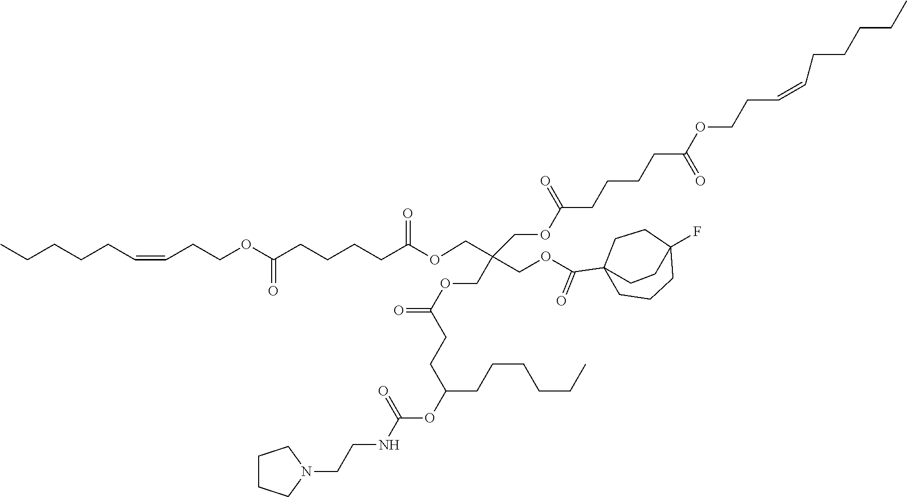 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCCC(F)(CC1)CC2] |
| 84 | 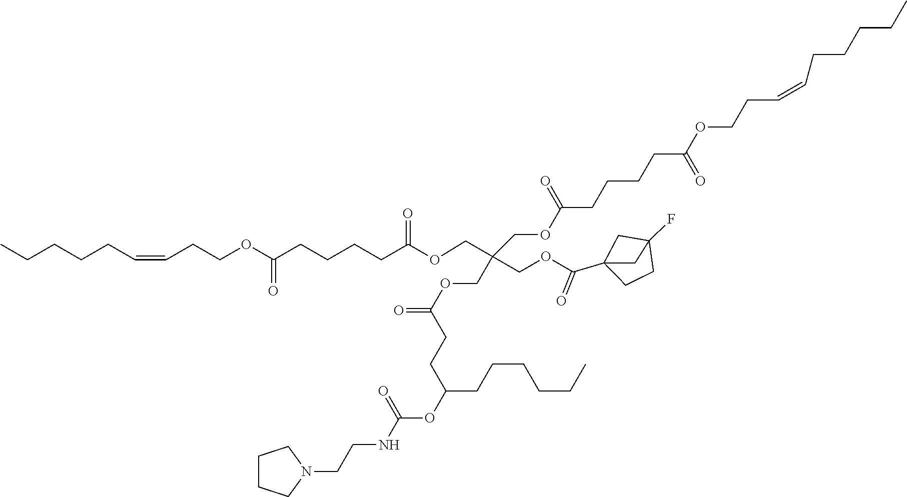 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(F)(C1)C2] |
| 85 | 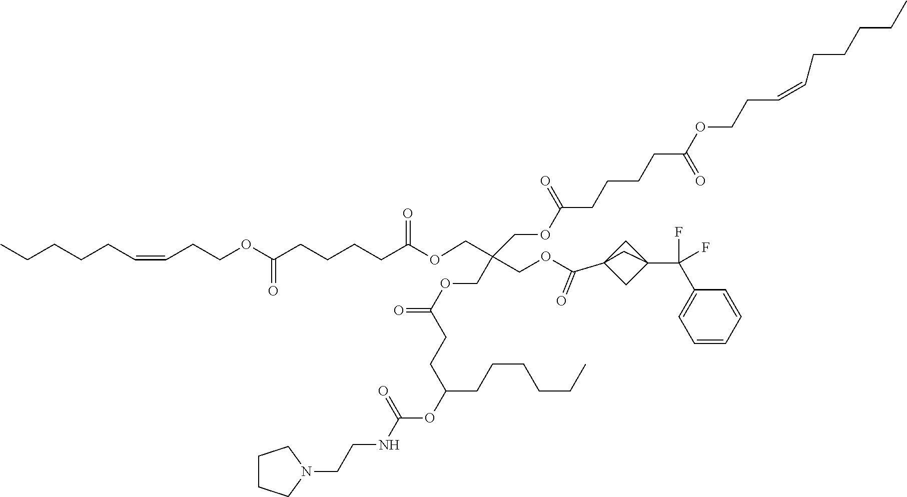 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C(F)(F)c3ccccc3)(C1)C2] |
| 86 | 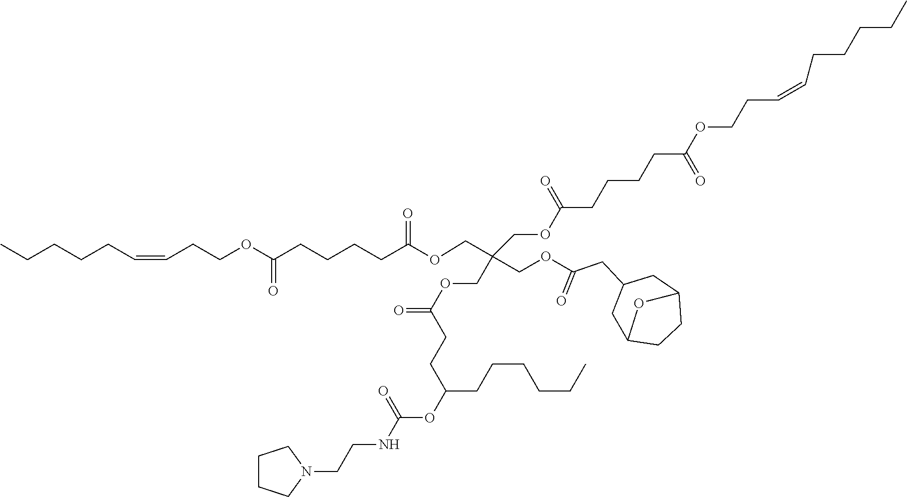 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC1CC2CCC(C1)O2] |
| 87 | 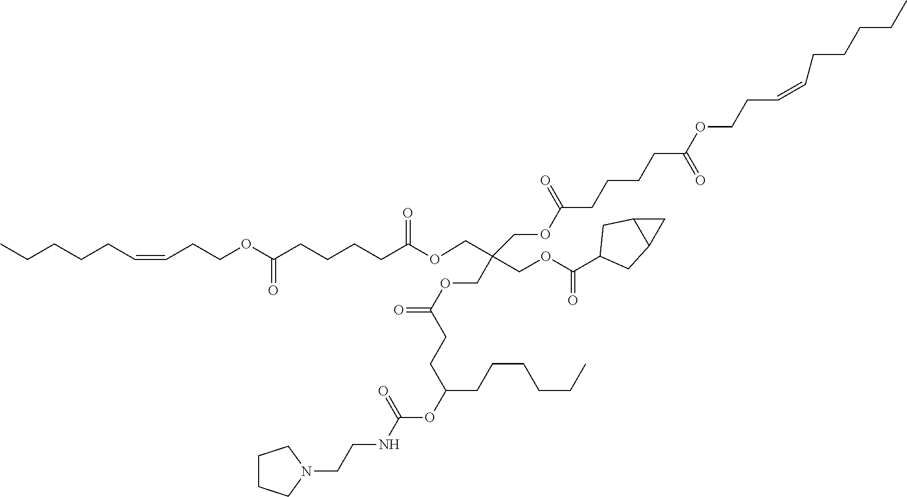 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CC2CC2C1] |
| 88 | 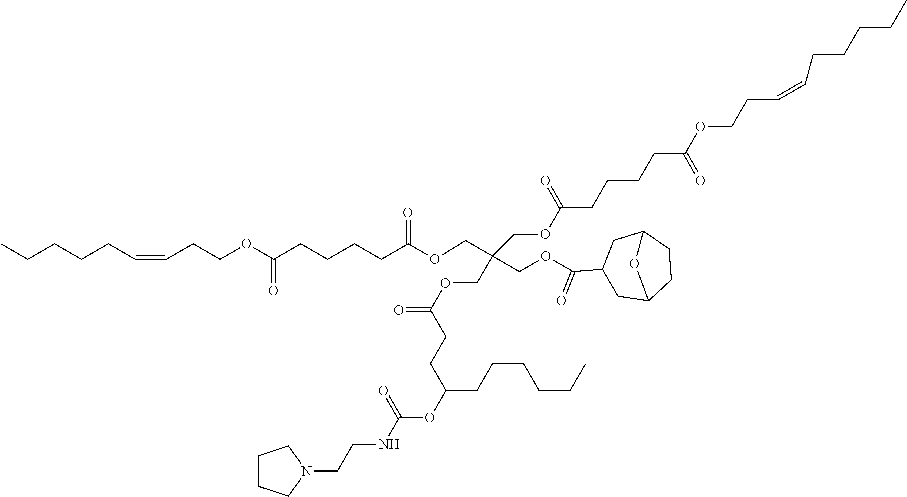 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CC2CCC(C1)O2] |
| 89 | 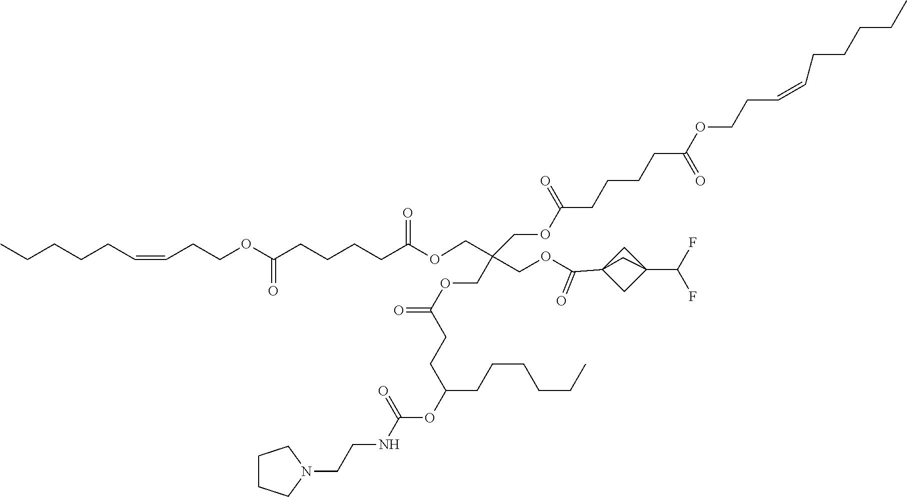 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C(F)F)(C1)C2] |
| 90 | 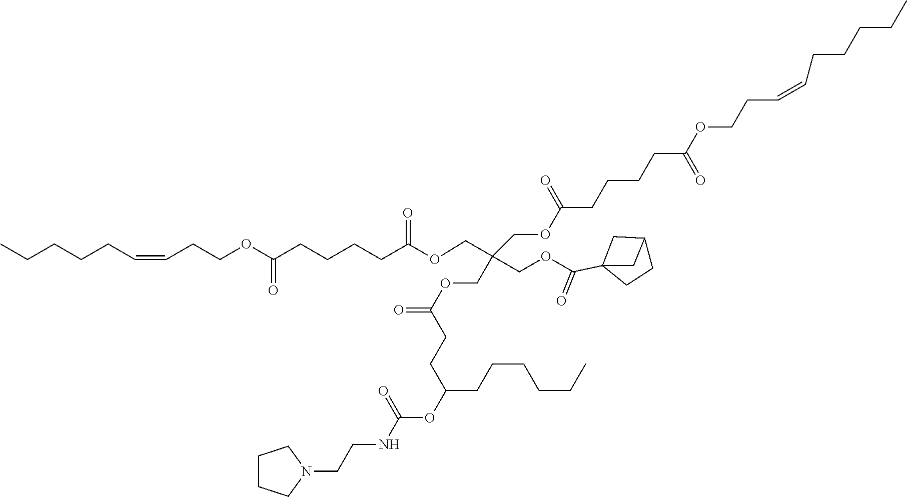 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(C1)C2] |
| 91 | 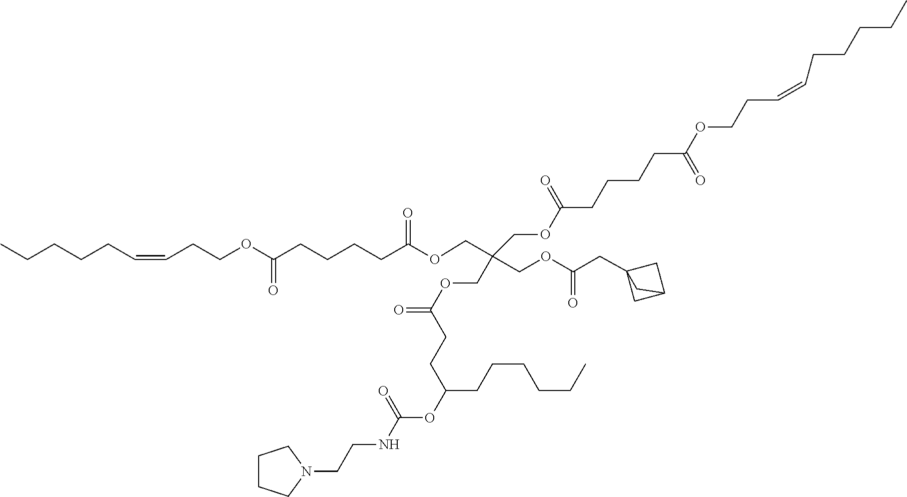 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)CC12CC(C1)C2] |
| 92 | 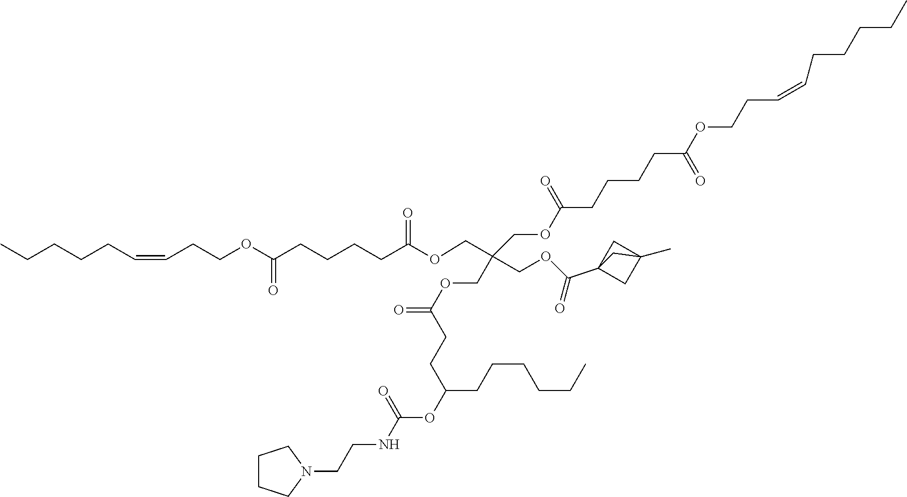 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC(C)(C1)C2] |
| 93 | 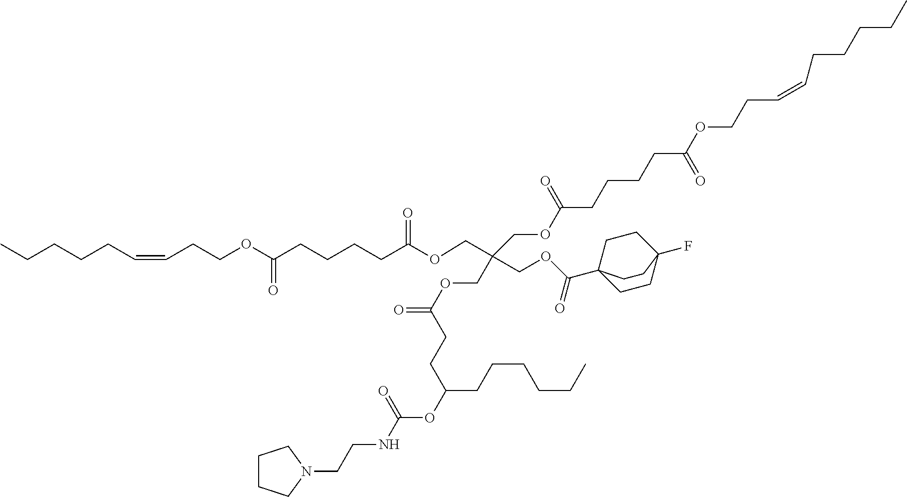 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(F)(CC1)CC2] |
| 94 | 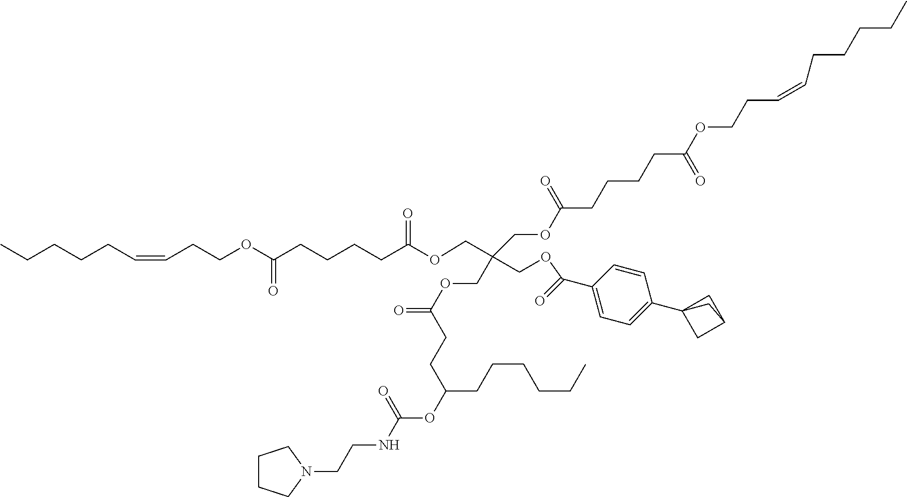 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)c1ccc(C23CC(C2)C3)cc1] |
| 95 | 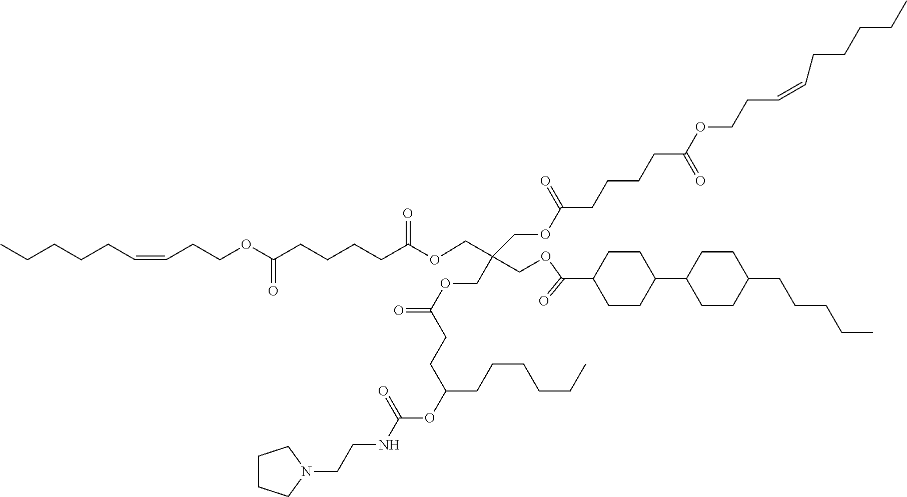 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CCC(C2CCC(CCCCC)CC2)CC1] |
| 96 | 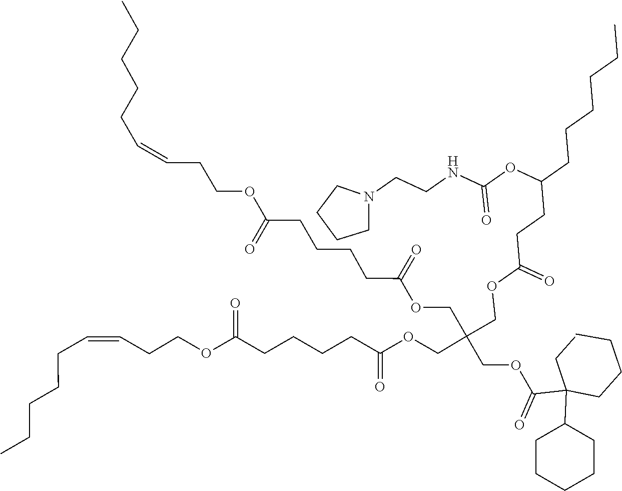 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C(CC)(CCCC)C1CCCCC1] |
| 97 | 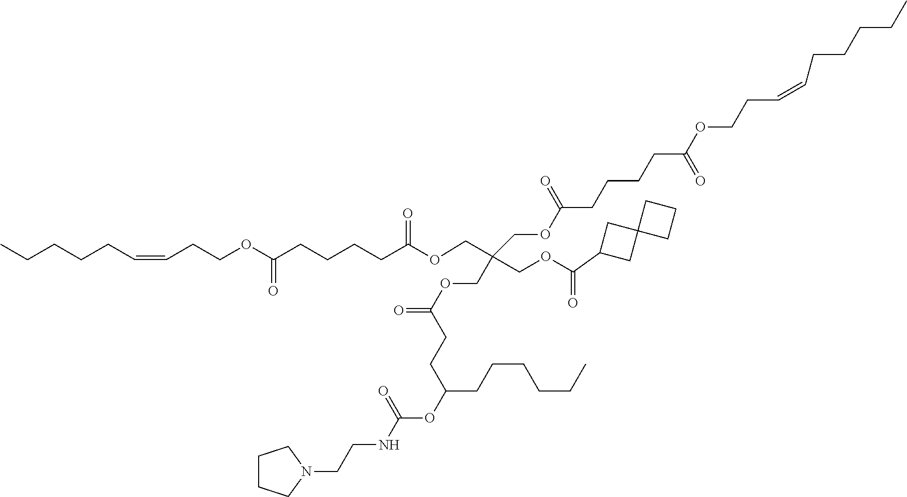 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CC2(CCC2)C1] |
| 98 | 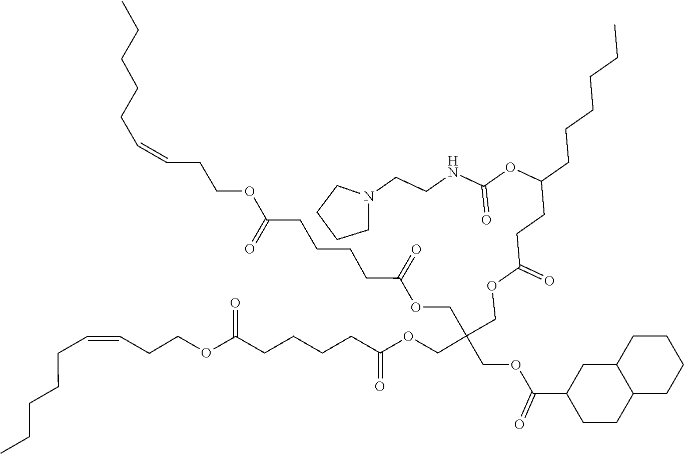 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CCC2CCCCC2C1] |
| 99 | 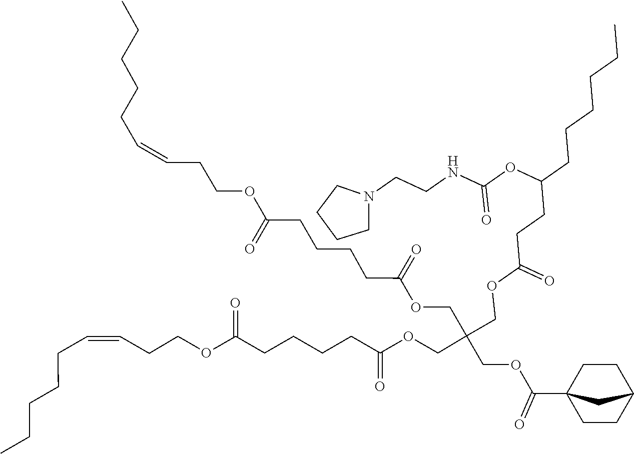 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)[C@]12CC[C@H](CC1)C2] |
| 100 | 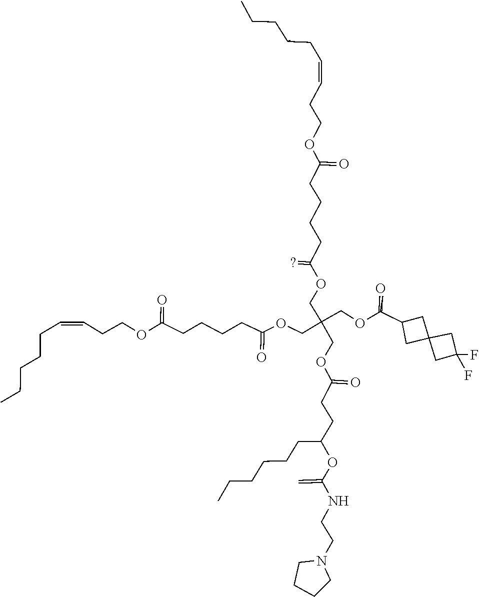 |
| 101 | 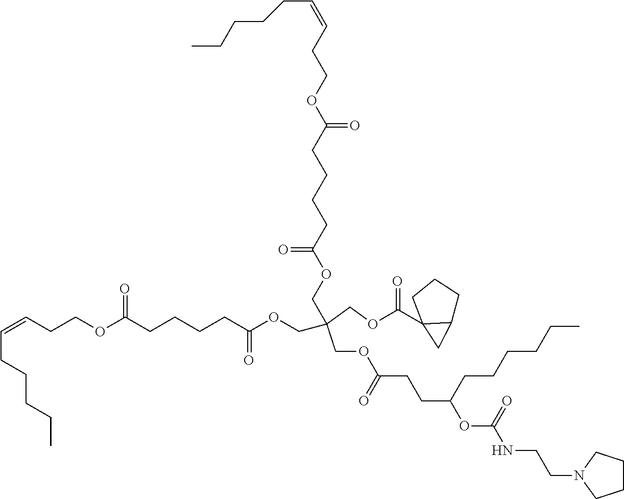 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCCC1C2] |
| 102 | 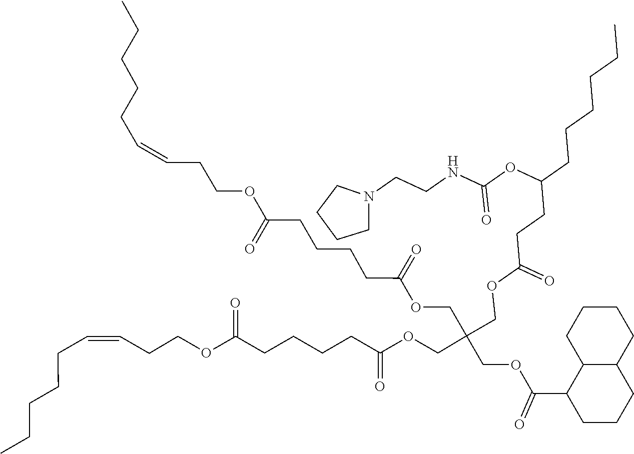 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CCCC2CCCCC21] |
| 103 | 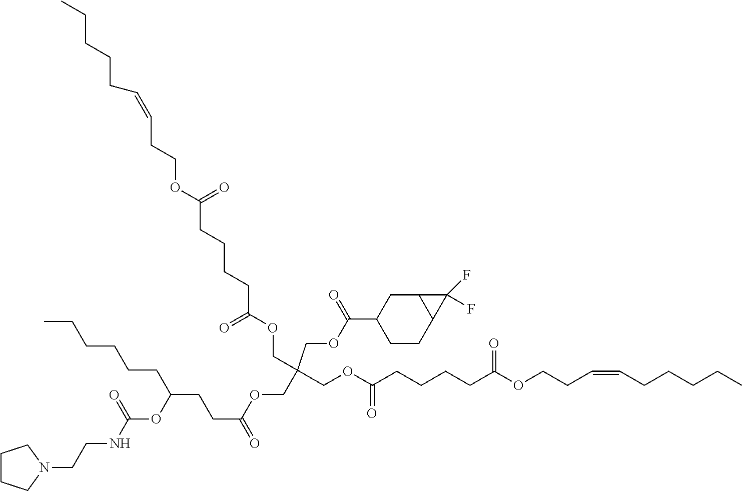 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CCC2C(C1)C2(F)F] |
| 104 | 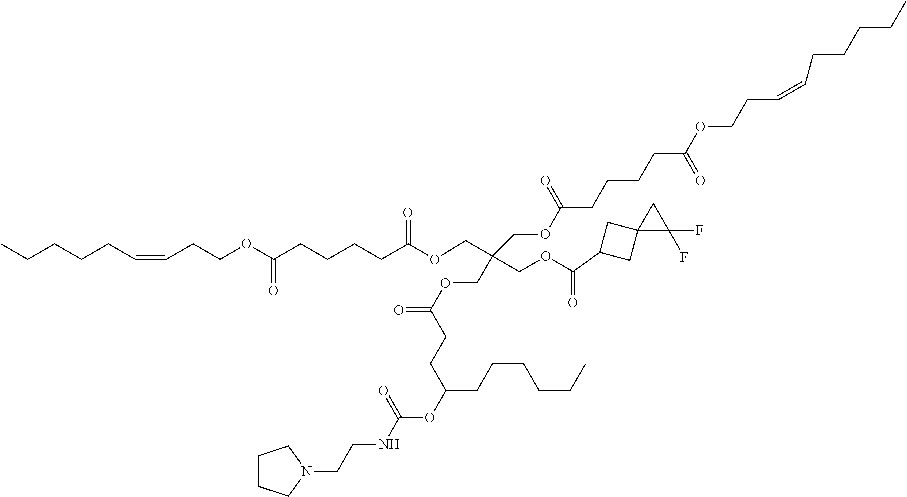 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C1CC2(C1)CC2(F)F] |
| 105 | 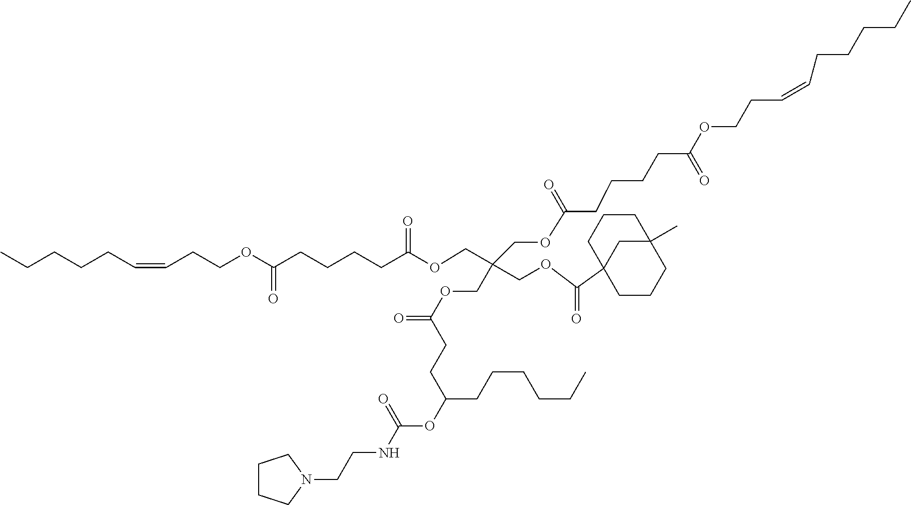 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCCC(C)(CCC1)C2] |
| 106 | 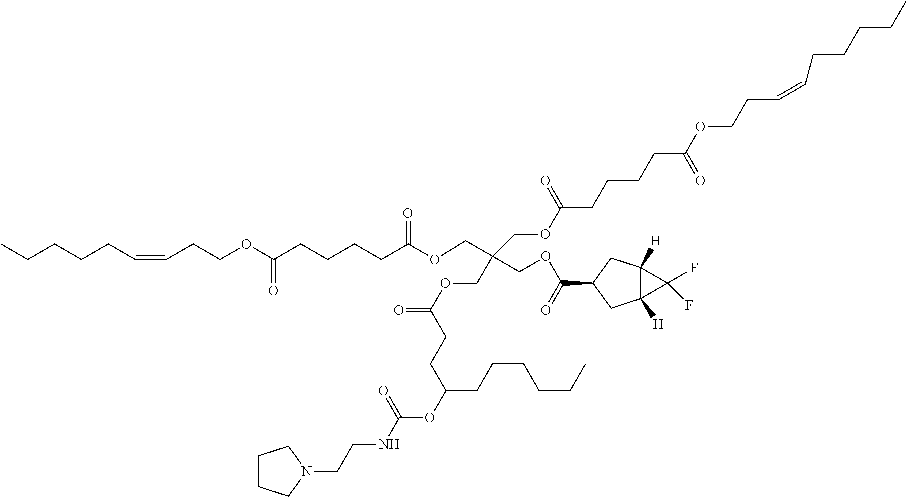 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)[C@@H]1C[C@@H]2[C@H](C1)C2(F)F] |
| 107 | 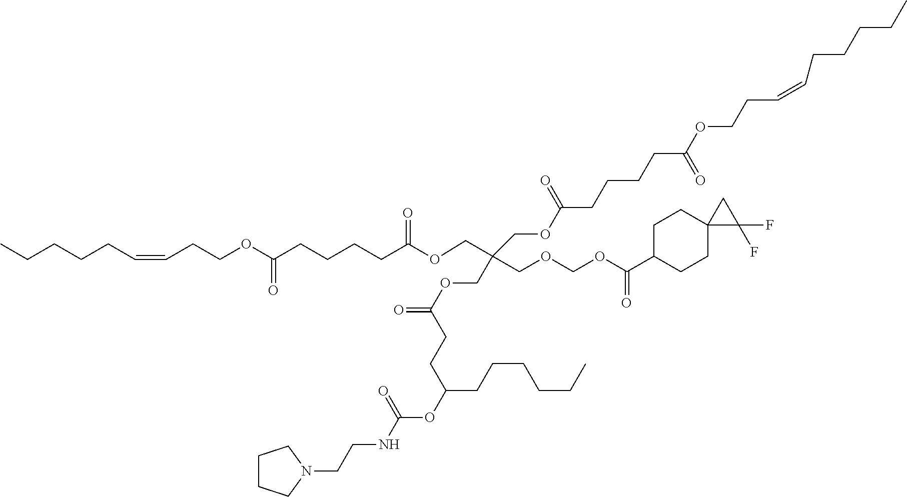 [SMILES: CCCCC/C=C\CCOC(=O)CCCCC(=O)OCC(COCOC(=O)C1CCC2(CC1)CC2(F)F)(COC(=O)CCCCC(=O)OCC/C=C\CCCCC)COC(=O)CCC(CCCCCC)OC(=O)NCCN1CCCC1] |
| 108 | 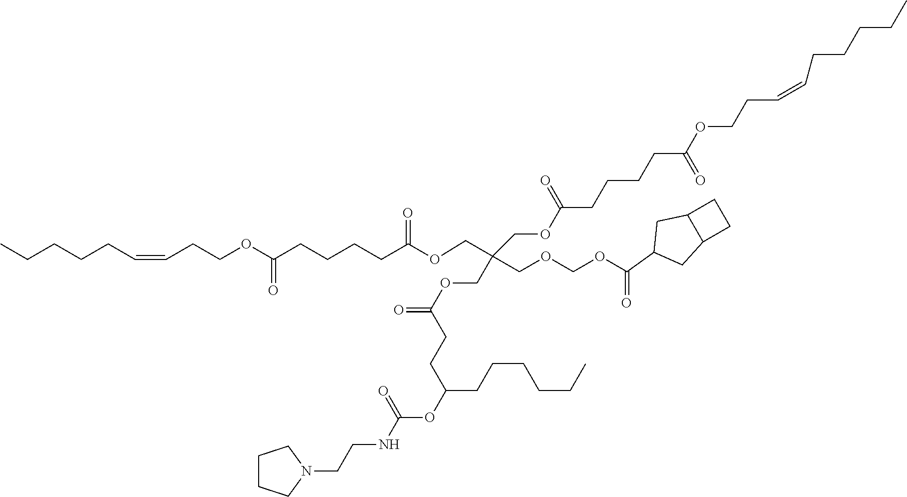 [SMILES: CCCCC/C=C\CCOC(=O)CCCCC(=O)OCC(COCOC(=O)C1CC2CCC2C1)(COC(=O)CCCCC(=O)OCC/C=C\CCCCC)COC(=O)CCC(CCCCCC)OC(=O)NCCN1CCCC1] |
| 109 | 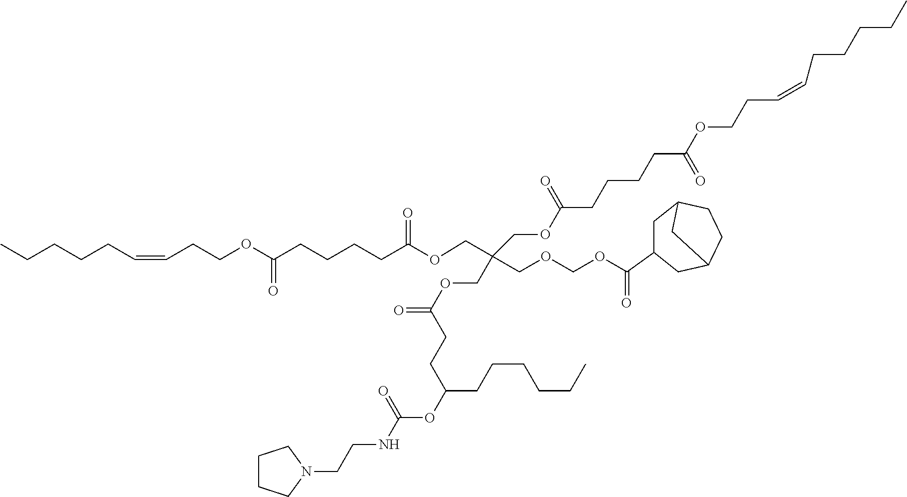 [SMILES: CCCCC/C=C\CCOC(=O)CCCCC(=O)OCC(COCOC(=O)C1CC2CCC(C2)C1)(COC(=O)CCCCC(=O)OCC/C=C\CCCCC)COC(=O)CCC(CCCCCC)OC(=O)NCCN1CCCC1] |
| 110 | 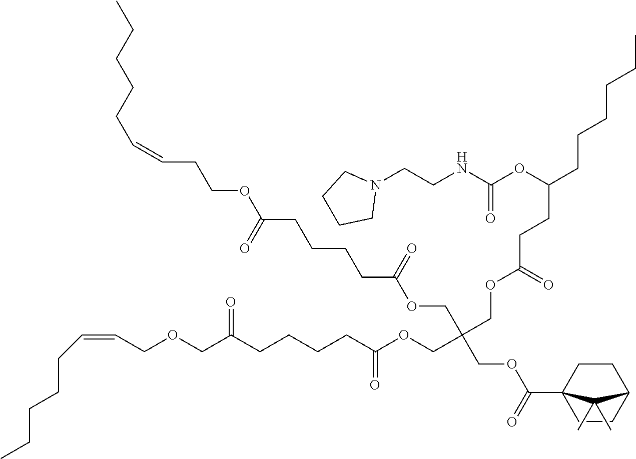 [SMILES: CCCCC/C=C\CCOC(=O)CCCCC(=O)OCC(COC(=O)CCCCC(=O)COC/C=C\CCCCC)(COC(=O)CCC(CCCCCC)OC(=O)NCCN1CCCC1)COC(=O)[C@]12CC[C@H](CC1)C2(C)C] |
| 111 | 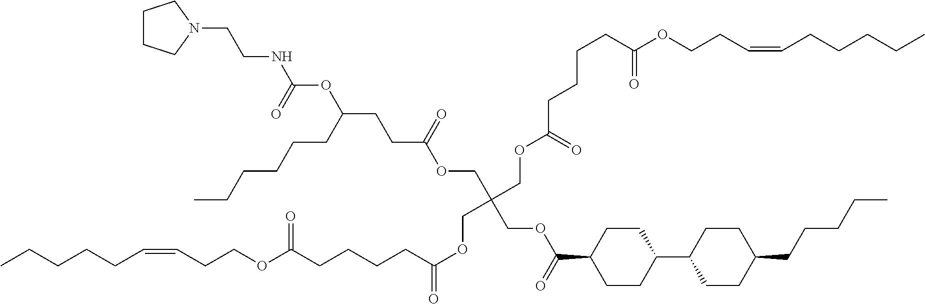 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)[C@H]1CC[C@H]([C@H]2CC[C@H](CCCCC)CC2)CC1] |
| 112 | 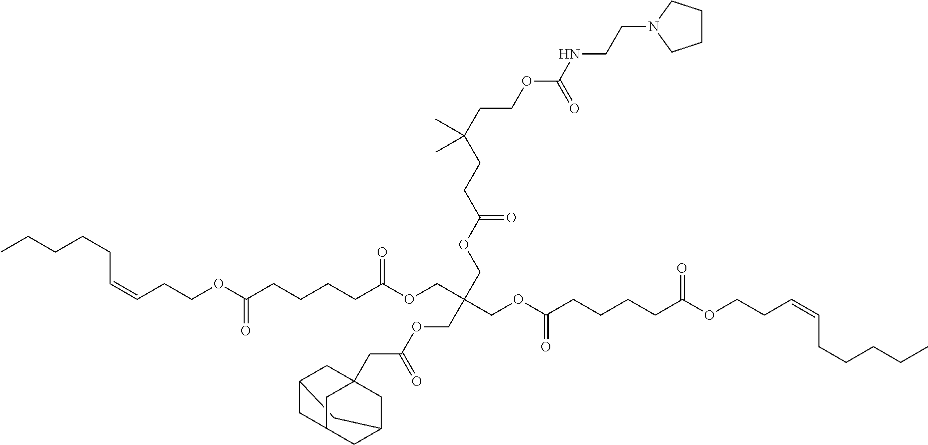 [SMILES: CCCCC/C=C\CCOC(=O)CCCCC(=O)OCC(COC(=O)CCCCC(=O)OCC/C=C\CCCCC)(COC(=O)CCC(C)(C)CCOC(=O)NCCN1CCCC1)COC(=O)CC12CC3CC(CC(C3)C1)C2] |
| 113 | 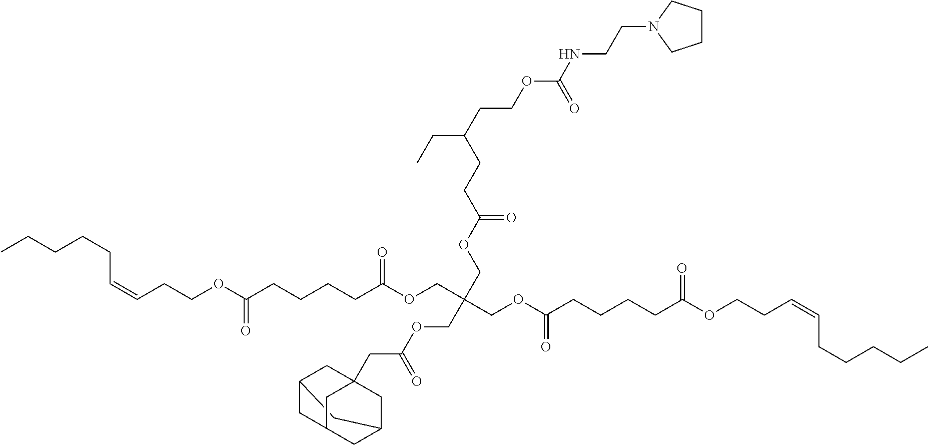 [SMILES: CCCCC/C=C\CCOC(=O)CCCCC(=O)OCC(COC(=O)CCCCC(=O)OCC/C=C\CCCCC)(COC(=O)CCC(CC)CCOC(=O)NCCN1CCCC1)COC(=O)CC12CC3CC(CC(C3)C1)C2] |
| 114 | 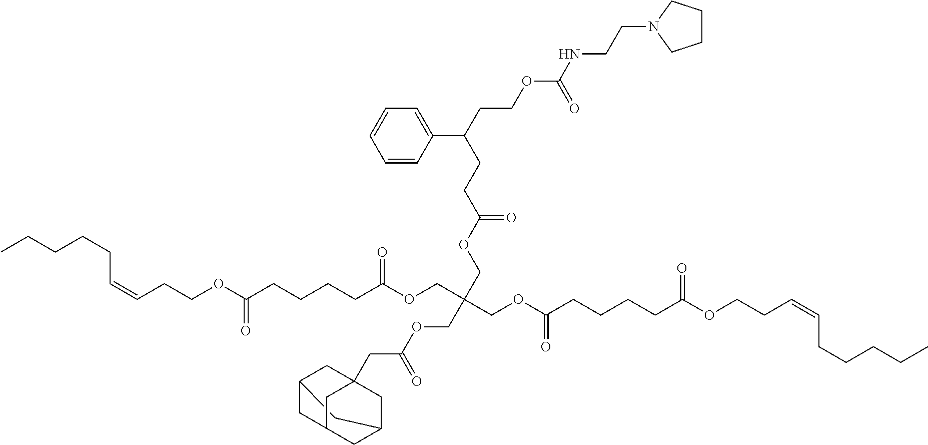 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)c1ccccc1)COC(=O)CC12CC3CC(CC(C3)C1)C2] |
| 115 | 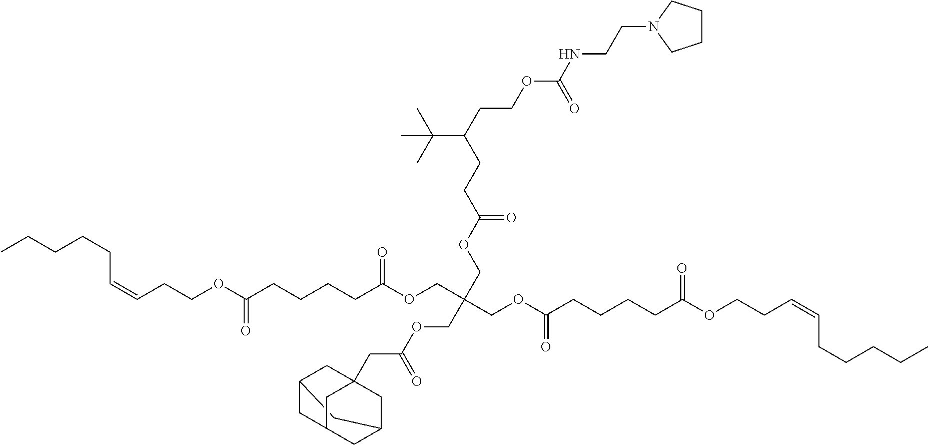 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)C(C)(C)C)COC(=O)CC12CC3CC(CC(C3)C1)C2] |
| 116 | 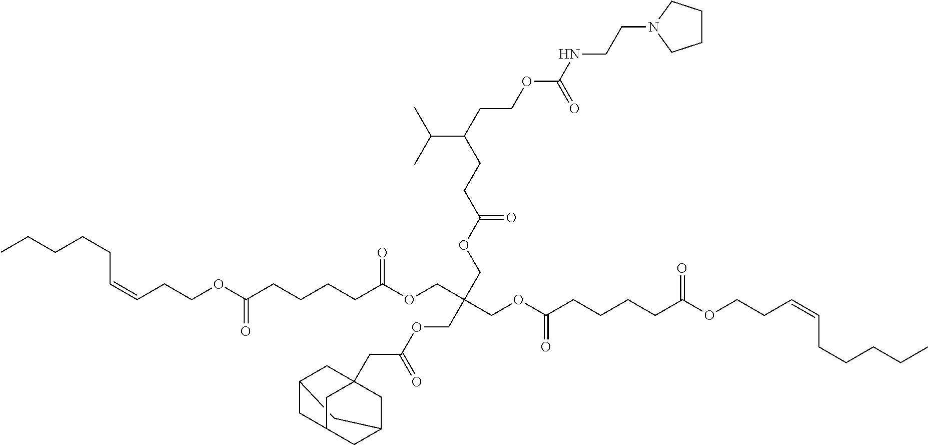 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)C(C)C)COC(=O)CC12CC3CC(CC(C3)C1)C2] |
| 117 | 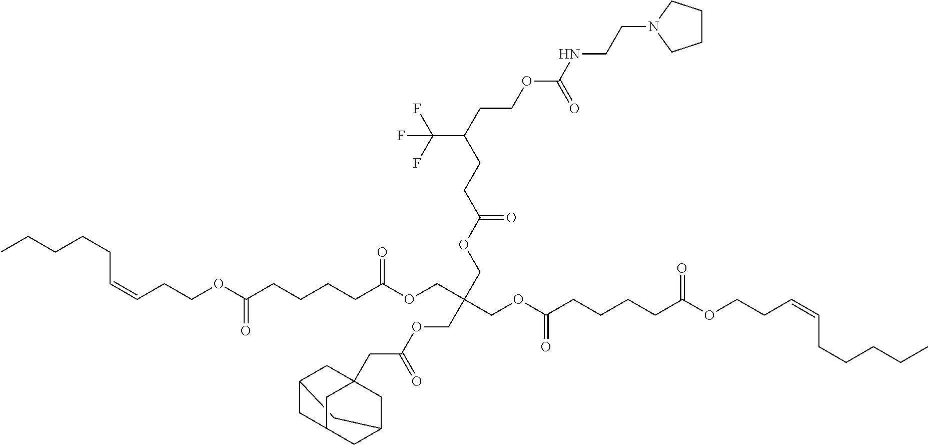 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)C(F)(F)F)COC(=O)CC12CC3CC(CC(C3)C1)C2] |
| 118 | 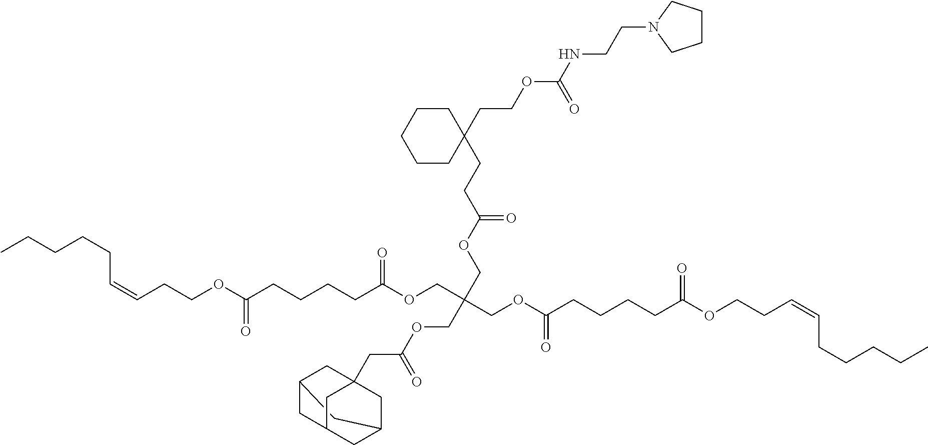 [SMILES: CCCCC/C=C\CCOC(=O)CCCCC(=O)OCC(COC(=O)CCCCC(=O)OCC/C=C\CCCCC)(COC(=O)CCC1(CCOC(=O)NCCN2CCCC2)CCCCC1)COC(=O)CC12CC3CC(CC(C3)C1)C2] |
| 119 | 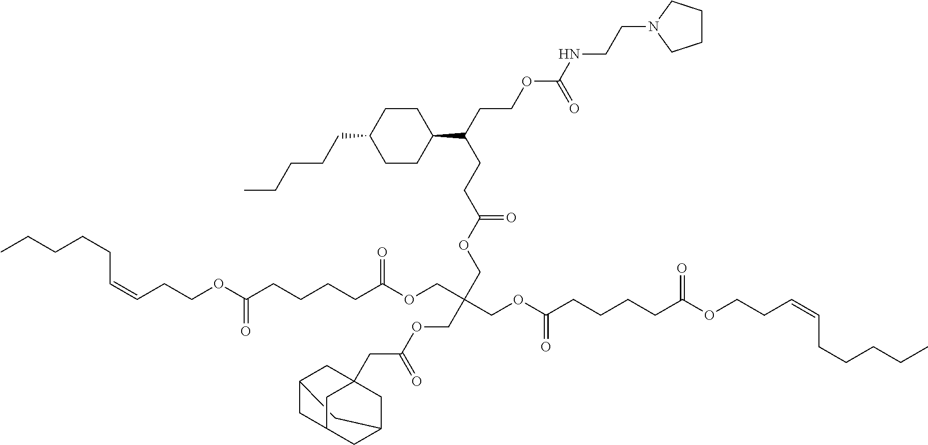 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)[C@H]1CC[C@H](CCCCC)CC1)COC(=O)CC12CC3CC(CC(C3)C1)C2] |
| 120 | 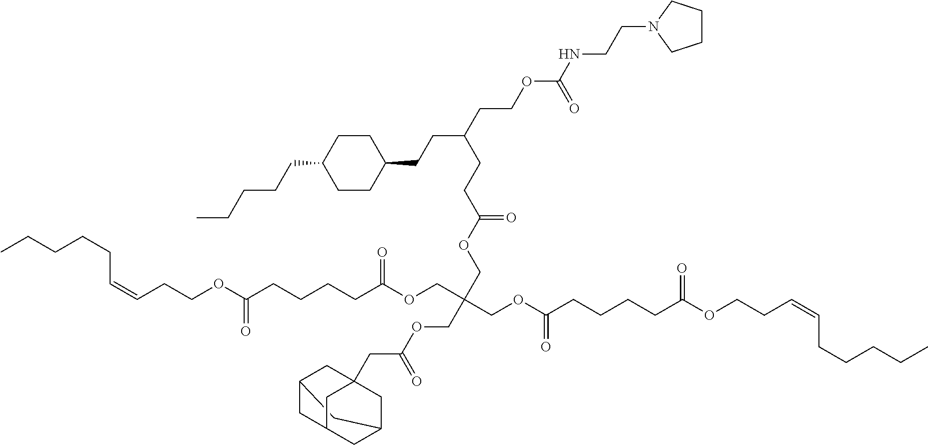 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)CC[C@H]1CC[C@H](CCCCC)CC1)COC(=O)CC12CC3CC(CC(C3)C1)C2] |
| 121 | 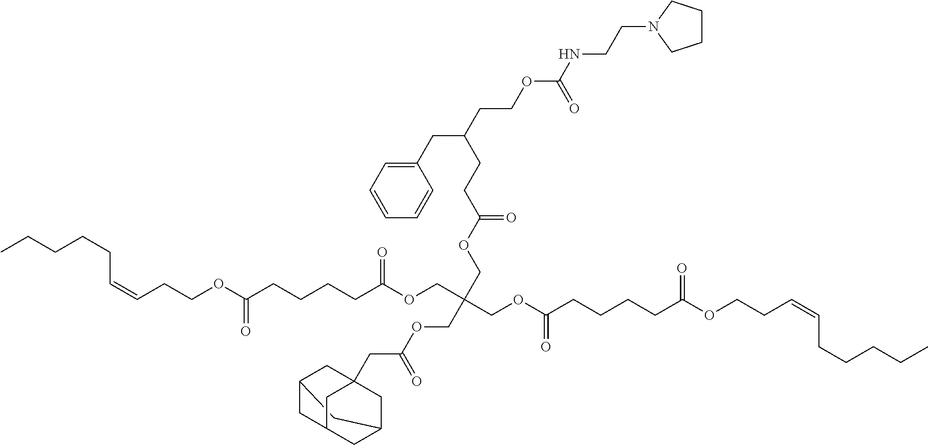 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)Cc1ccccc1)COC(=O)CC12CC3CC(CC(C3)C1)C2] |
| 122 | 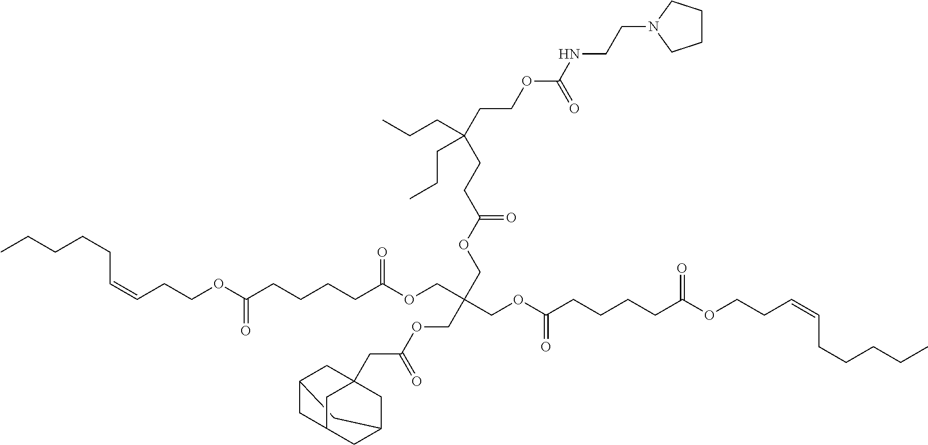 [SMILES: CCCCC/C=C\CCOC(=O)CCCCC(=O)OCC(COC(=O)CCCCC(=O)OCC/C=C\CCCCC)(COC(=O)CCC(CCC)(CCC)CCOC(=O)NCCN1CCCC1)COC(=O)CC12CC3CC(CC(C3)C1)C2] |
| 123 | 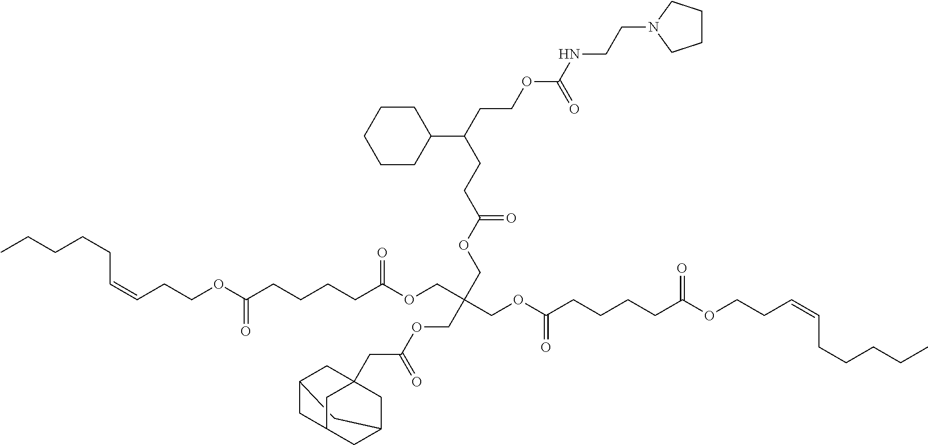 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)C1CCCCC1)COC(=O)CC12CC3CC(CC(C3)C1)C2] |
| 124 | 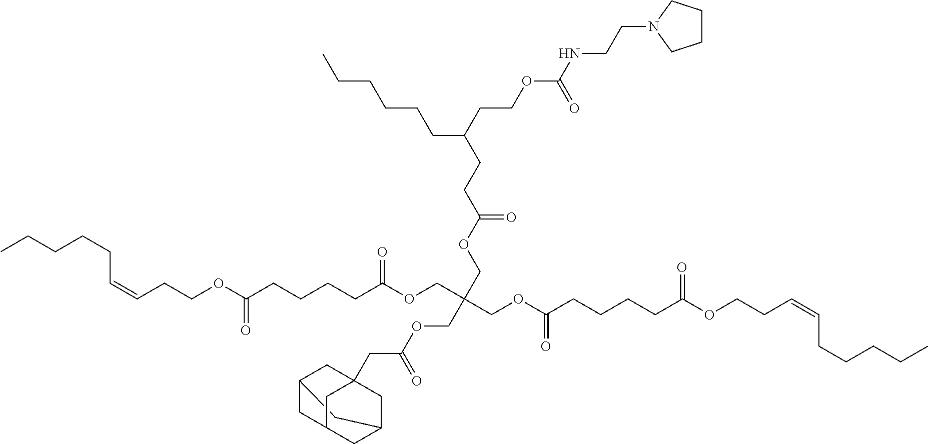 [SMILES: CCCCC/C=C\CCOC(=O)CCCCC(=O)OCC(COC(=O)CCCCC(=O)OCC/C=C\CCCCC)(COC(=O)CCC(CCCCCC)CCOC(=O)NCCN1CCCC1)COC(=O)CC12CC3CC(CC(C3)C1)C2] |
| 125 | 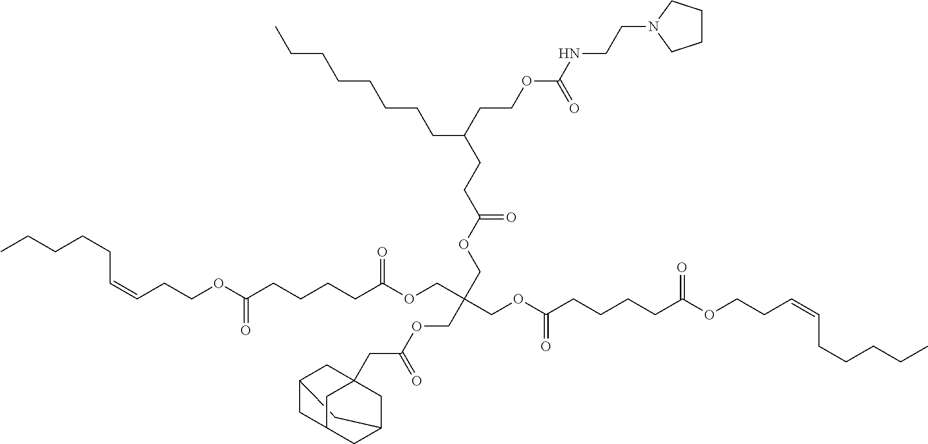 [SMILES: CCCCC/C=C\CCOC(=O)CCCCC(=O)OCC(COC(=O)CCCCC(=O)OCC/C=C\CCCCC)(COC(=O)CCC(CCCCCCCC)CCOC(=O)NCCN1CCCC1)COC(=O)CC12CC3CC(CC(C3)C1)C2] |
| 126 | 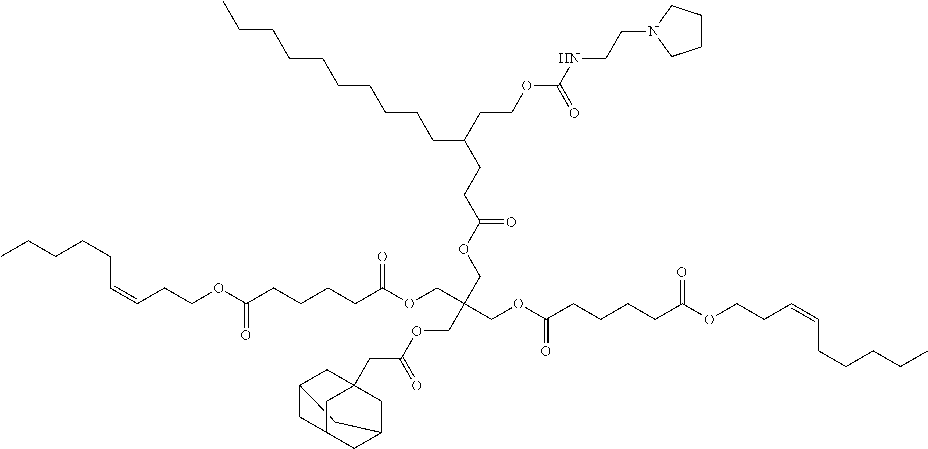 [SMILES: CCCCC/C=C\CCOC(=O)CCCCC(=O)OCC(COC(=O)CCCCC(=O)OCC/C=C\CCCCC)(COC(=O)CCC(CCCCCCCCCC)CCOC(=O)NCCN1CCCC1)COC(=O)CC12CC3CC(CC(C3)C1)C2] |
| 127 | 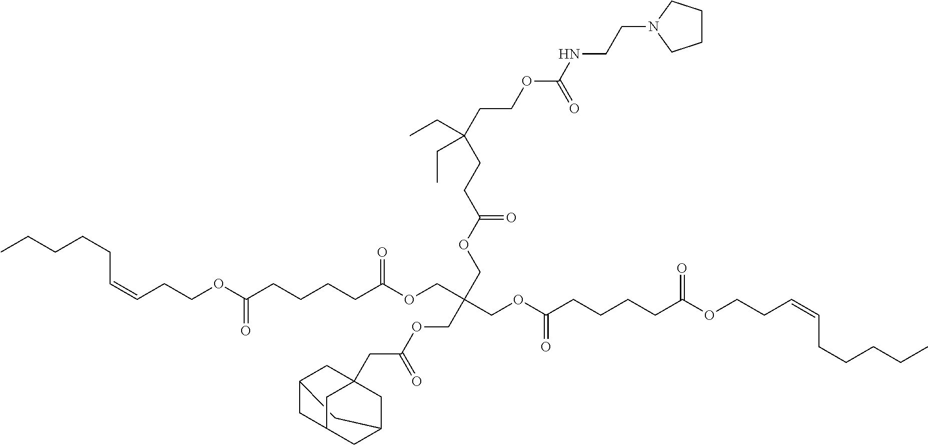 [SMILES: CCCCC/C=C\CCOC(=O)CCCCC(=O)OCC(COC(=O)CCCCC(=O)OCC/C=C\CCCCC)(COC(=O)CCC(CC)(CC)CCOC(=O)NCCN1CCCC1)COC(=O)CC12CC3CC(CC(C3)C1)C2] |
| 128 | 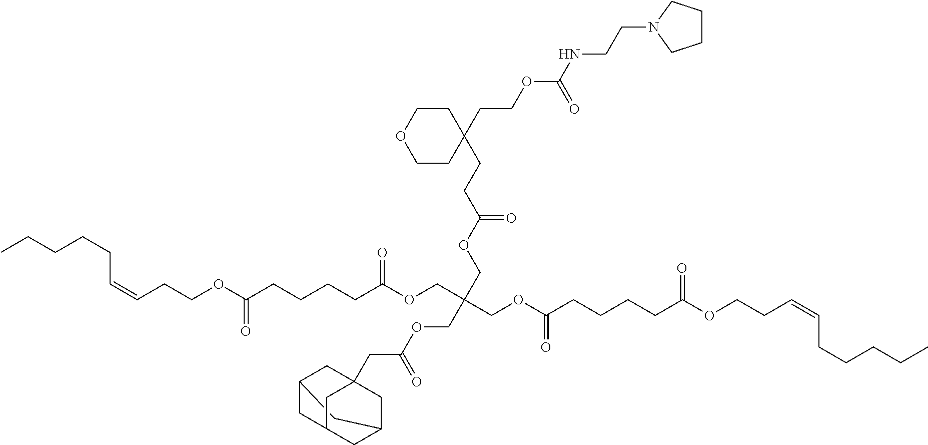 [SMILES: CCCCC/C=C\CCOC(=O)CCCCC(=O)OCC(COC(=O)CCCCC(=O)OCC/C=C\CCCCC)(COC(=O)CCC1(CCOC(=O)NCCN2CCCC2)CCOCC1)COC(=O)CC12CC3CC(CC(C3)C1)C2] |
| 129 | 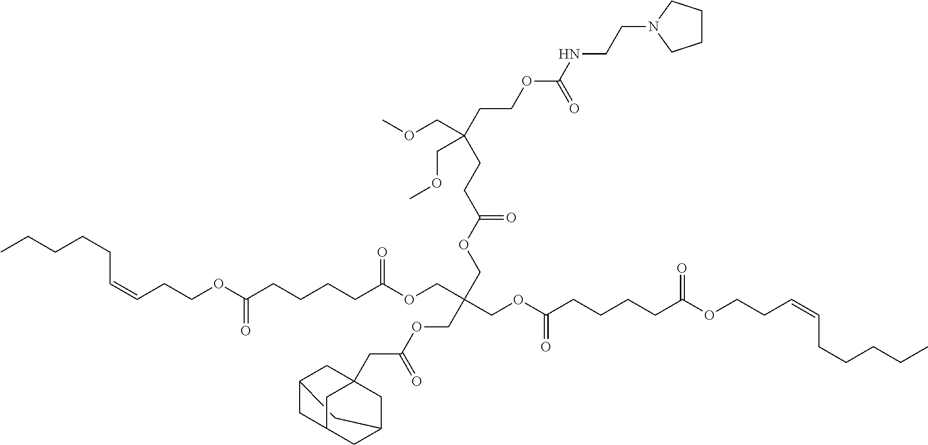 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)(COC)COC)COC(=O)CC12CC3CC(CC(C3)C1)C2] |
| 130 | 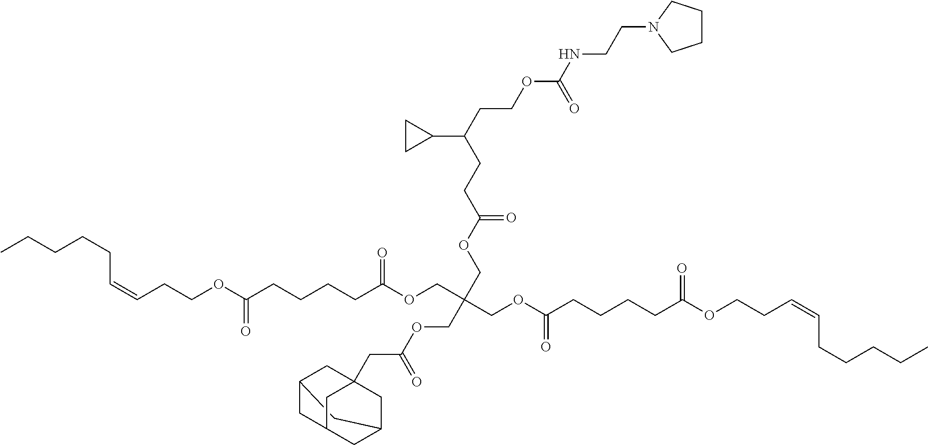 [SMILES: CCCCC/C=C\CCOC(=O)CCCCC(=O)OCC(COC(=O)CCCCC(=O)OCC/C=C\CCCCC)(COC(=O)CCC(CCOC(=O)NCCN1CCCC1)C1CC1)COC(=O)CC12CC3CC(CC(C3)C1)C2] |
| 131 | 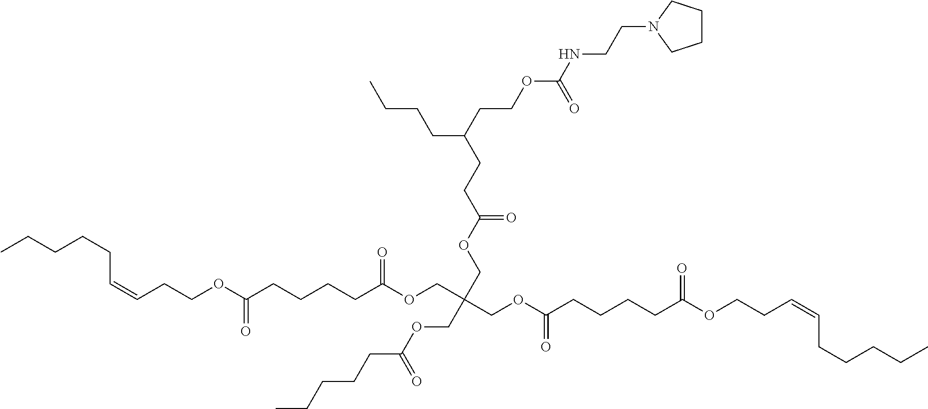 [SMILES: CCCCC/C=C\CCOC(=O)CCCCC(=O)OCC(COC(=O)CCCCC)(COC(=O)CCCCC(=O)OCC/C=C\CCCCC)COC(=O)CCC(CCCC)CCOC(=O)NCCN1CCCC1] |
| 132 | 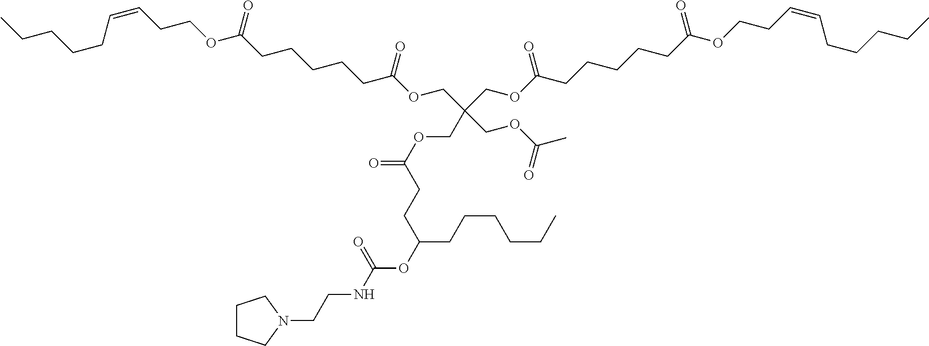 [SMILES: CCCCC/C=C\CCOC(=O)CCCCCC(=O)OCC(COC(C)=O)(COC(=O)CCCCCC(=O)OCC/C=C\CCCCC)COC(=O)CCC(CCCCCC)OC(=O)NCCN1CCCC1] |
| 133 | 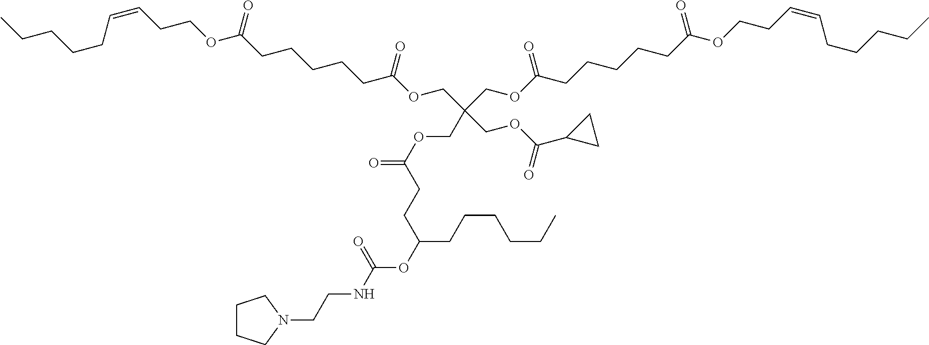 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)C1CC1] |
| 134 | 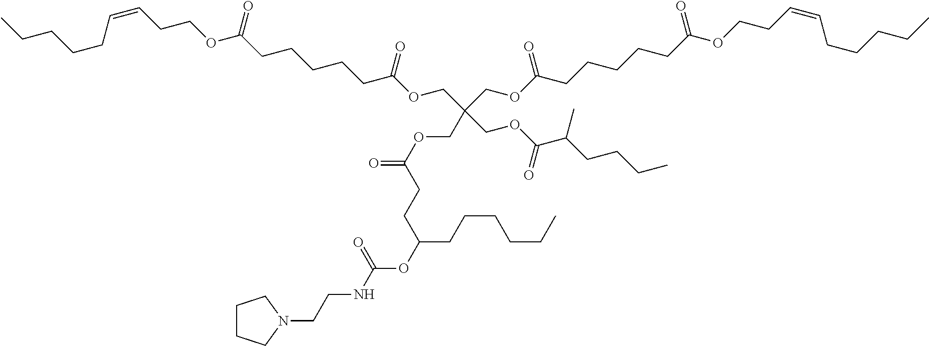 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)C(C)CCCC] |
| 135 | 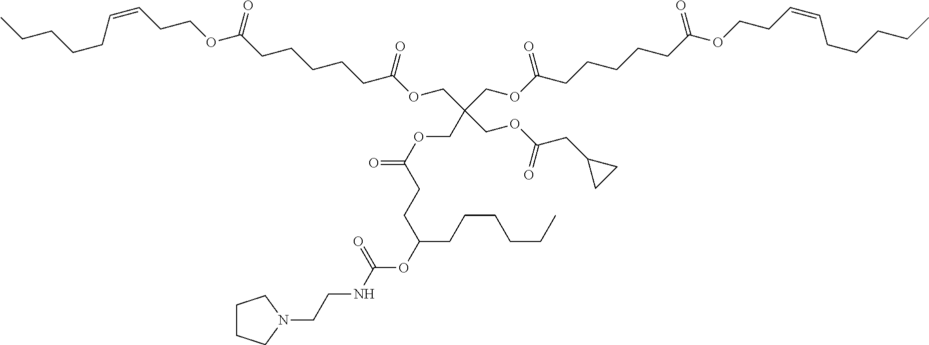 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)CC1CC1] |
| 136 | 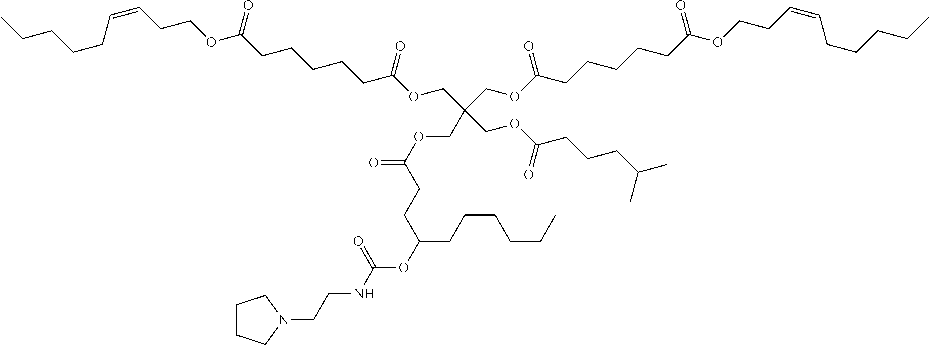 [SMILES: CCCCC/C=C\CCOC(=O)CCCCCC(=O)OCC(COC(=O)CCCCCC(=O)OCC/C=C\CCCCC)(COC(=O)CCCC(C)C)COC(=O)CCC(CCCCCC)OC(=O)NCCN1CCCC1] |
| 137 | 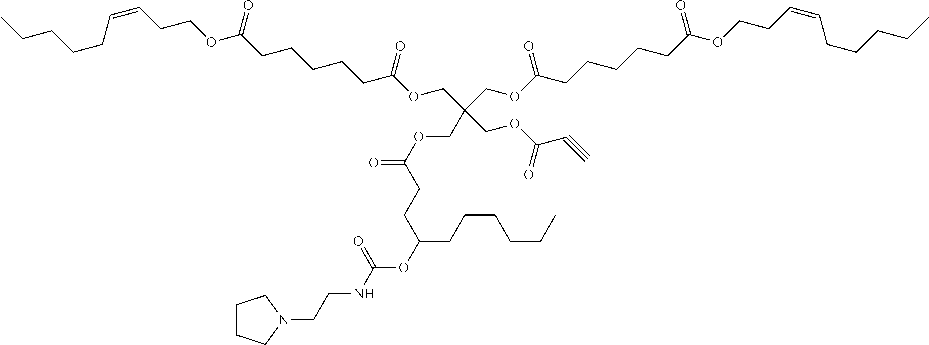 [SMILES: C#CC(=O)OCC(COC(=O)CCCCCC(=O)OCC/C=C\CCCCC)(COC(=O)CCCCCC(=O)OCC/C=C\CCCCC)COC(=O)CCC(CCCCCC)OC(=O)NCCN1CCCC1] |
| 138 | 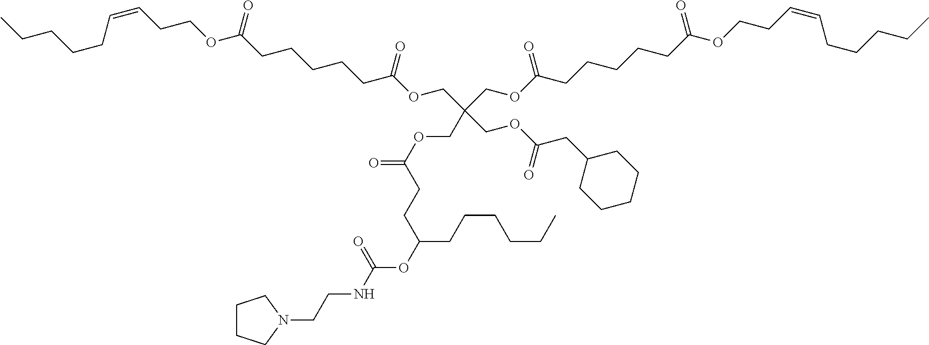 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)CC1CCCCC1] |
| 139 | 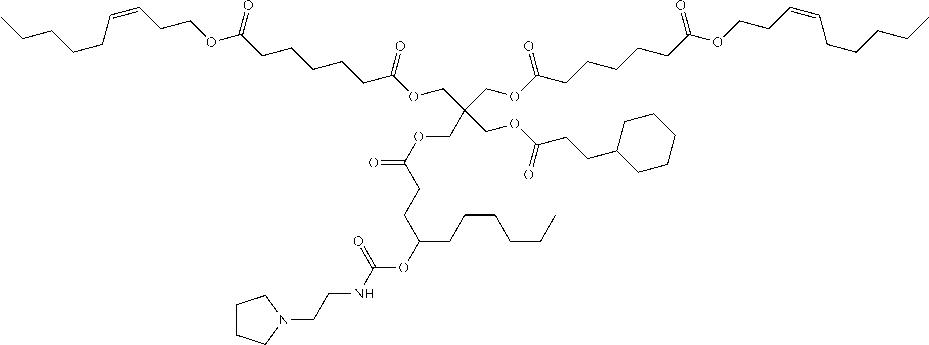 [SMILES: CCCCC/C=C\CCOC(=O)CCCCCC(=O)OCC(COC(=O)CCCCCC(=O)OCC/C=C\CCCCC)(COC(=O)CCC1CCCCC1)COC(=O)CCC(CCCCCC)OC(=O)NCCN1CCCC1] |
| 140 | 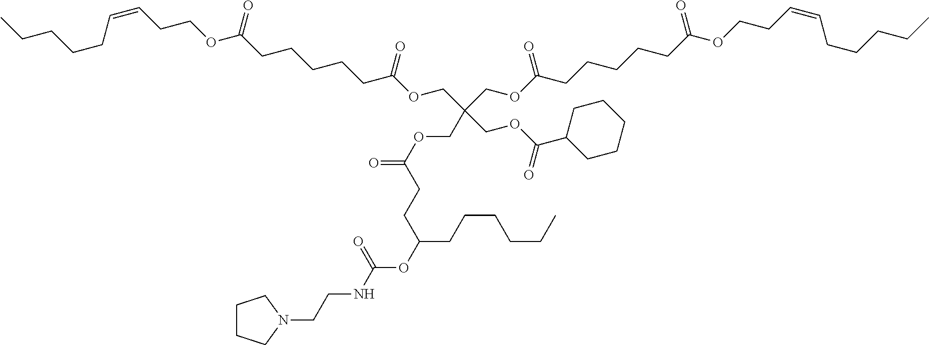 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)C1CCCCC1] |
| 141 | 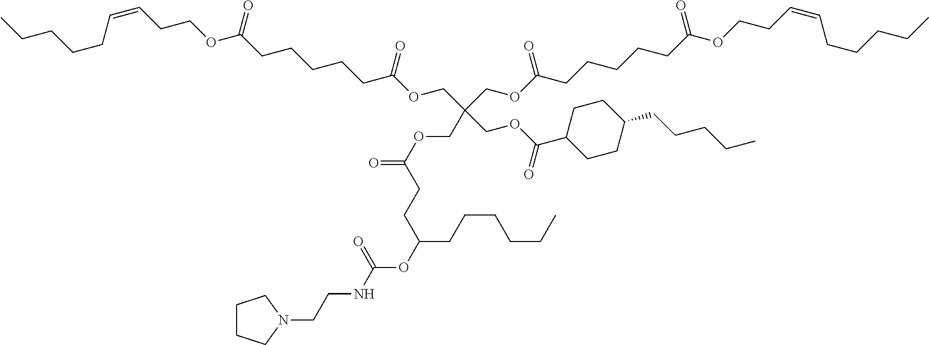 |
| 142 | 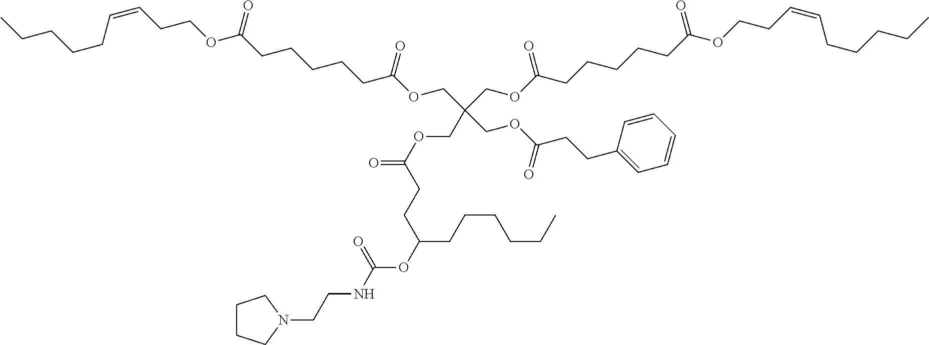 [SMILES: CCCCC/C=C\CCOC(=O)CCCCCC(=O)OCC(COC(=O)CCCCCC(=O)OCC/C=C\CCCCC)(COC(=O)CCc1ccccc1)COC(=O)CCC(CCCCCC)OC(=O)NCCN1CCCC1] |
| 143 | 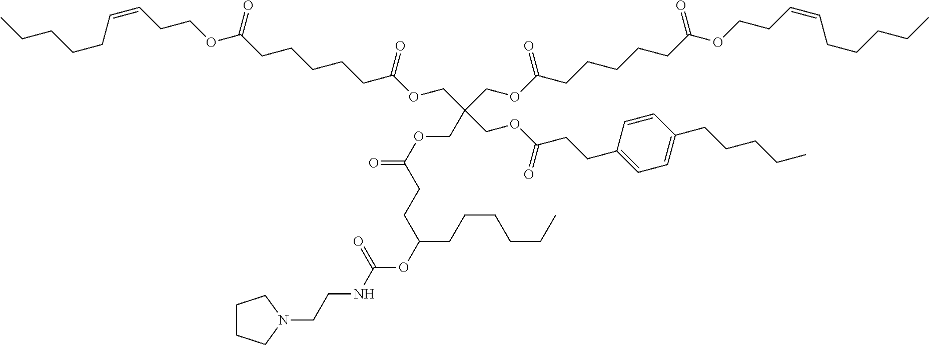 [SMILES: CCCCC/C=C\CCOC(=O)CCCCCC(=O)OCC(COC(=O)CCCCCC(=O)OCC/C=C\CCCCC)(COC(=O)CCc1ccc(CCCCC)cc1)COC(=O)CCC(CCCCCC)OC(=O)NCCN1CCCC1] |
| 144 | 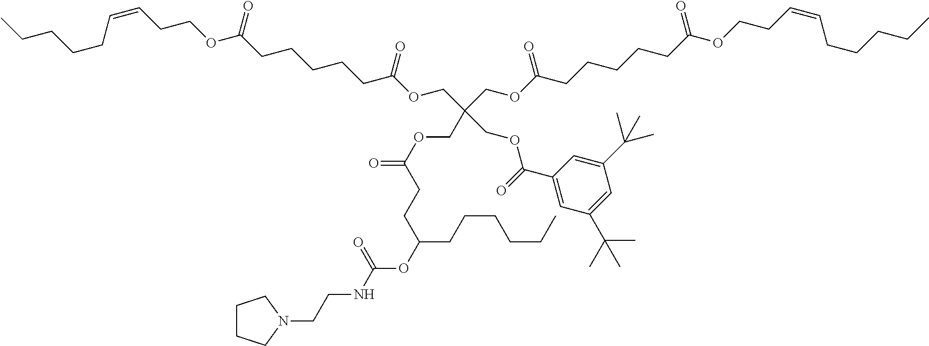 [SMILES: CCCCC/C=C\CCOC(=O)CCCCCC(=O)OCC(COC(=O)CCCCCC(=O)OCC/C=C\CCCCC)(COC(=O)CCC(CCCCCC)OC(=O)NCCN1CCCC1)COC(=O)c1cc(C(C)(C)C)cc(C(C)(C)C)c1] |
| 145 | 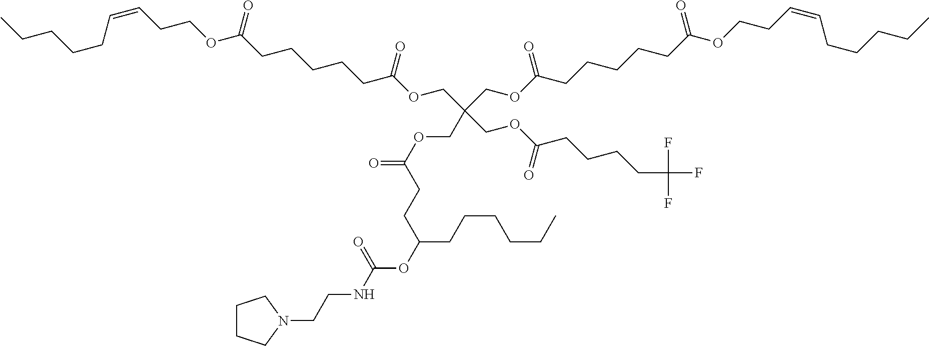 [SMILES: CCCCC/C=C\CCOC(=O)CCCCCC(=O)OCC(COC(=O)CCCCCC(=O)OCC/C=C\CCCCC)(COC(=O)CCCCC(F)(F)F)COC(=O)CCC(CCCCCC)OC(=O)NCCN1CCCC1] |
| 146 | 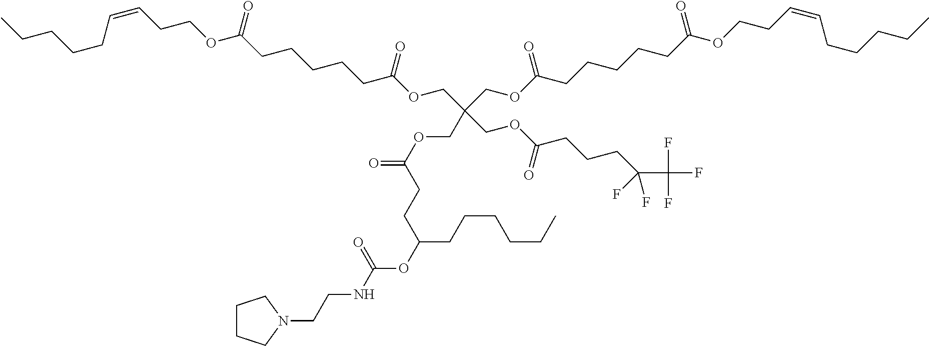 [SMILES: CCCCC/C=C\CCOC(=O)CCCCCC(=O)OCC(COC(=O)CCCCCC(=O)OCC/C=C\CCCCC)(COC(=O)CCCC(F)(F)C(F)(F)F)COC(=O)CCC(CCCCCC)OC(=O)NCCN1CCCC1] |
| 147 | 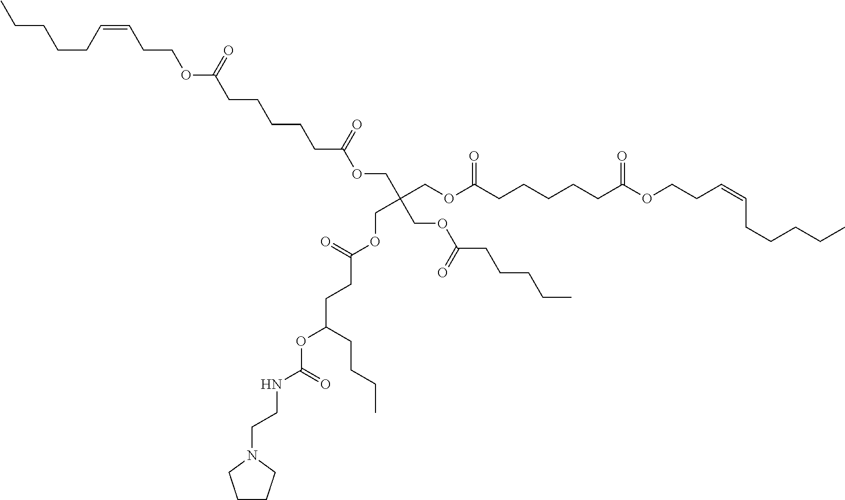 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCCC)OC(=O)NCCN1CCCC1] |
| 148 | 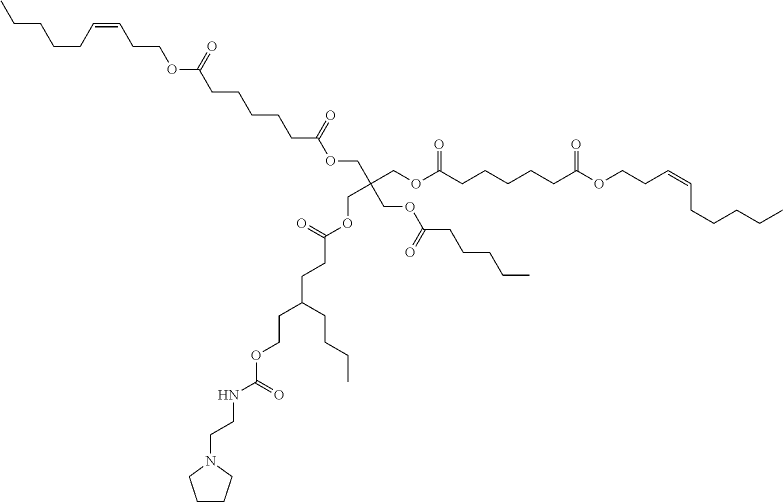 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCCC)CCOC(=O)NCCN1CCCC1] |
| 149 | 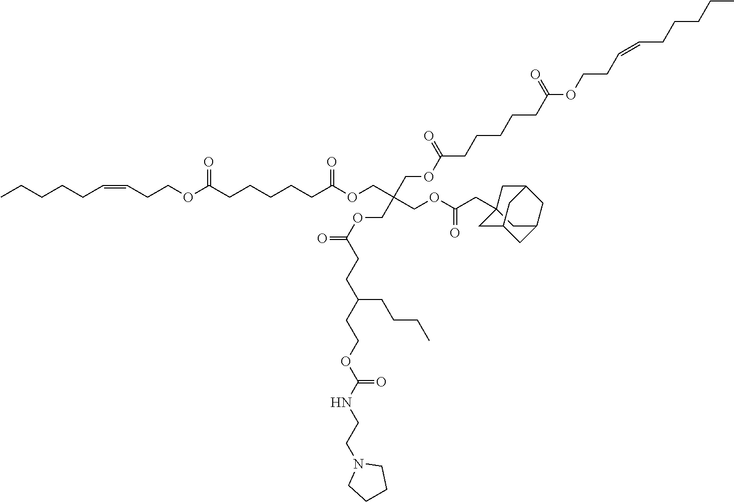 [SMILES: CCCCC/C=C\CCOC(=O)CCCCCC(=O)OCC(COC(=O)CCCCCC(=O)OCC/C=C\CCCCC)(COC(=O)CCC(CCCC)CCOC(=O)NCCN1CCCC1)COC(=O)CC12CC3CC(CC(C3)C1)C2] |
| 150 | 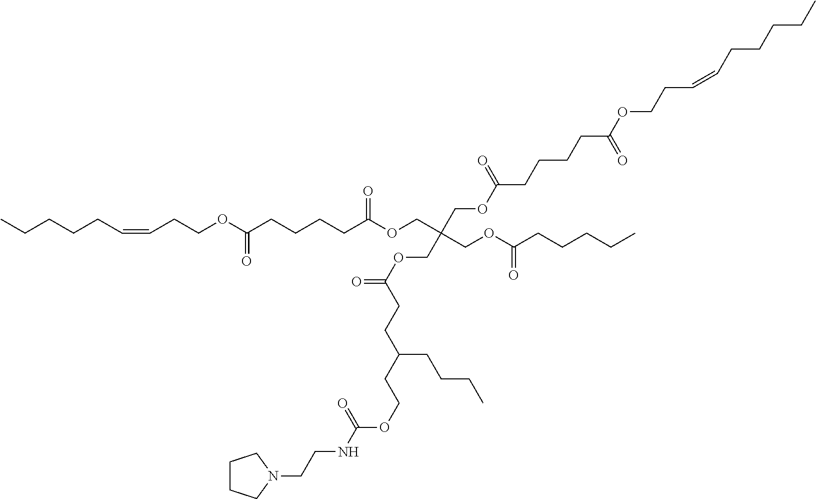 [SMILES: CCCCC/C=C\CCOC(=O)CCCCC(=O)OCC(COC(=O)CCCCC)(COC(=O)CCCCC(=O)OCC/C=C\CCCCC)COC(=O)CCC(CCCC)CCOC(=O)NCCN1CCCC1] |
| 151 | 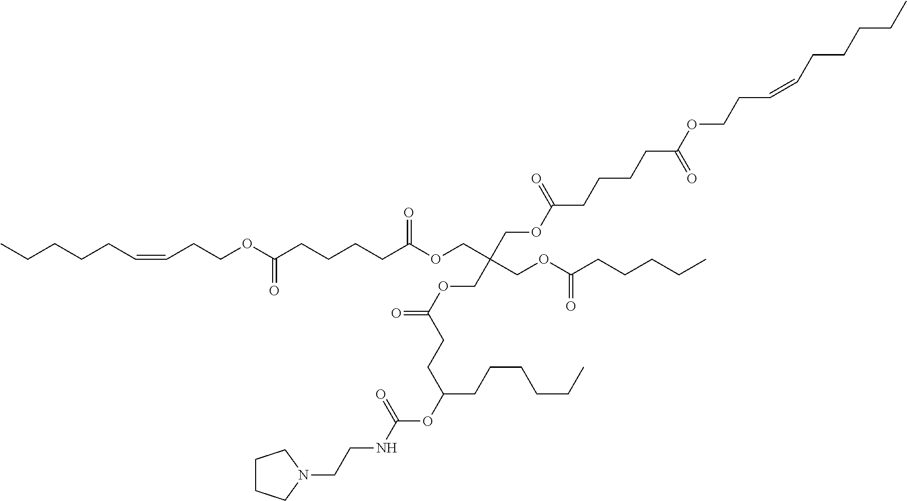 [SMILES: CCCCC/C=C\CCOC(=O)CCCCC(=O)OCC(COC(=O)CCCCC)(COC(=O)CCCCC(=O)OCC/C=C\CCCCC)COC(=O)CCC(CCCCCC)OC(=O)NCCN1CCCC1] |
| 152 | 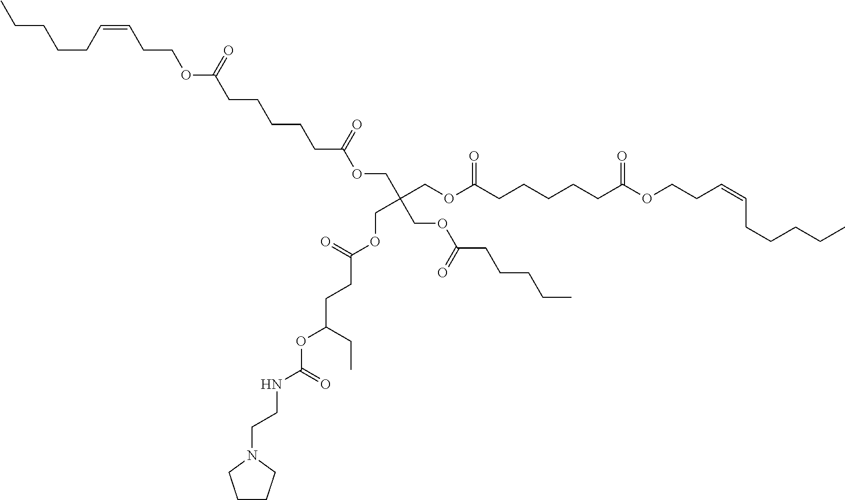 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CC)OC(=O)NCCN1CCCC1] |
| 153 | 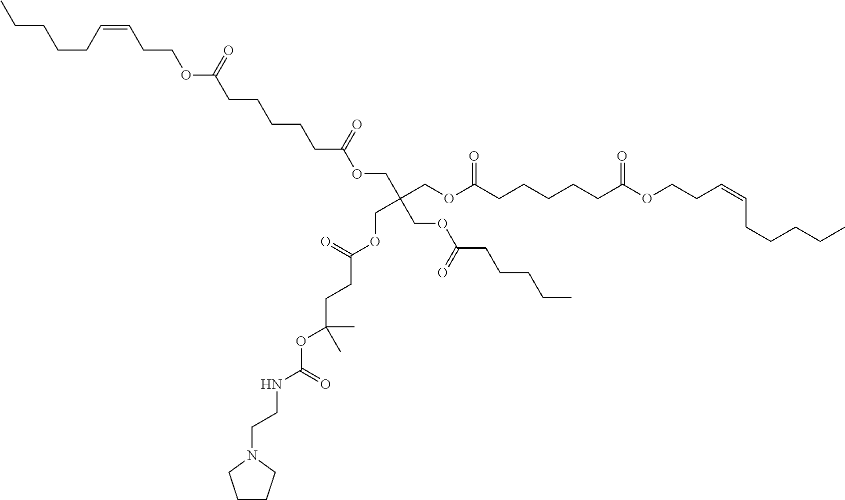 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(C)(C)OC(=O)NCCN1CCCC1] |
| 154 | 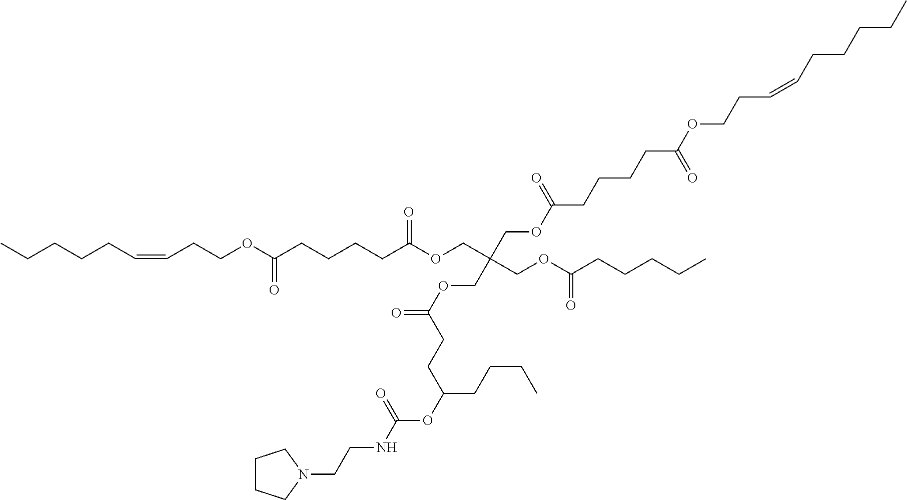 [SMILES: CCCCC/C=C\CCOC(=O)CCCCC(=O)OCC(COC(=O)CCCCC)(COC(=O)CCCCC(=O)OCC/C=C\CCCCC)COC(=O)CCC(CCCC)OC(=O)NCCN1CCCC1] |
| 155 | 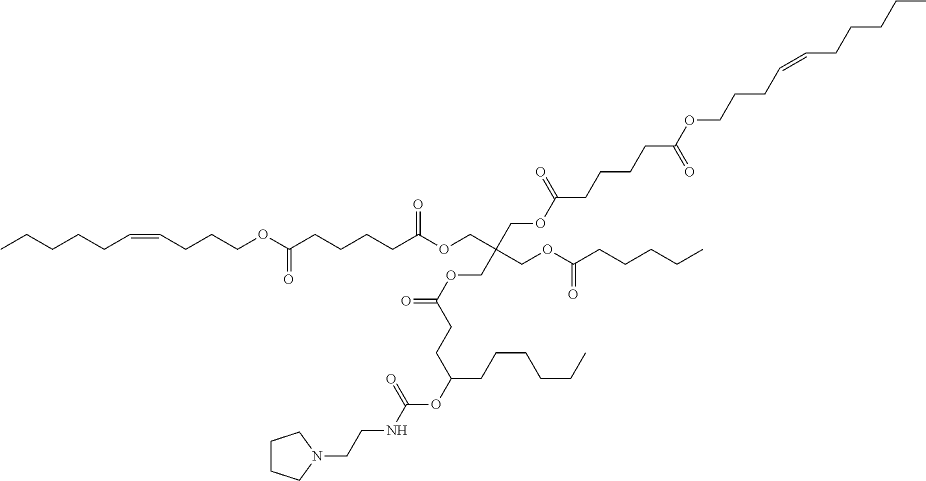 [SMILES: CCCCC/C=C\CCCOC(=O)CCCCC(=O)OCC(COC(=O)CCCCC)(COC(=O)CCCCC(=O)OCCC/C=C\CCCCC)COC(=O)CCC(CCCCCC)OC(=O)NCCN1CCCC1] |
| 156 | 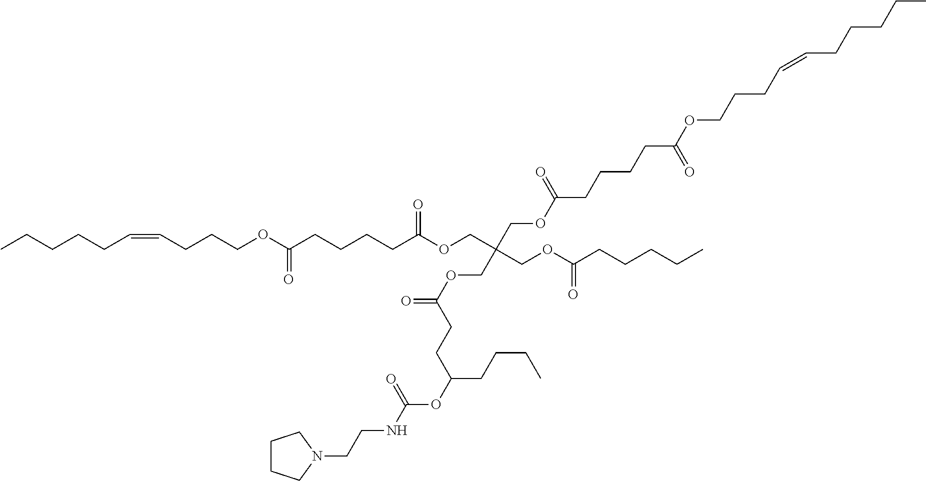 [SMILES: CCCCC/C=C\CCCOC(=O)CCCCC(=O)OCC(COC(=O)CCCCC)(COC(=O)CCCCC(=O)OCCC/C=C\CCCCC)COC(=O)CCC(CCCC)OC(=O)NCCN1CCCC1] |
| 157 | 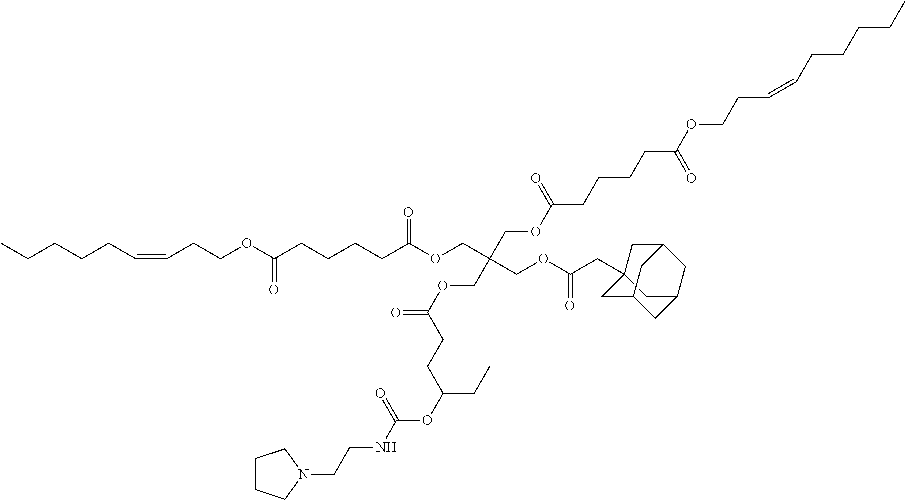 [SMILES: CCCCC/C=C\CCOC(=O)CCCCC(=O)OCC(COC(=O)CCCCC(=O)OCC/C=C\CCCCC)(COC(=O)CCC(CC)OC(=O)NCCN1CCCC1)COC(=O)CC12CC3CC(CC(C3)C1)C2] |
| 158 | 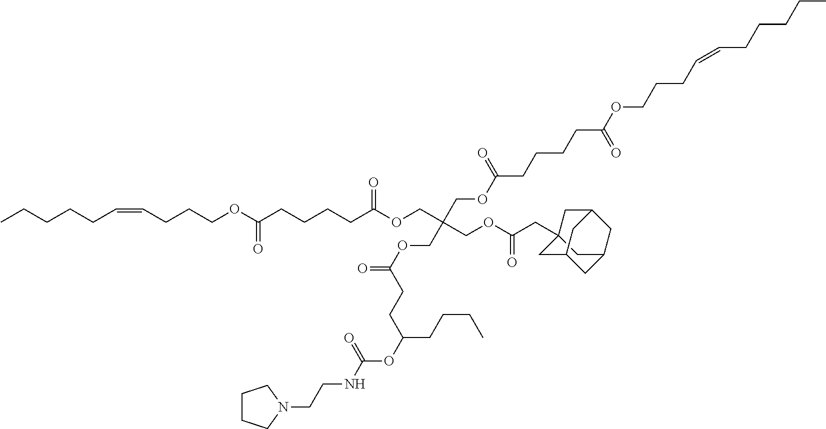 [SMILES: CCCCC/C=C\CCCOC(=O)CCCCC(=O)OCC(COC(=O)CCCCC(=O)OCCC/C=C\CCCCC)(COC(=O)CCC(CCCC)OC(=O)NCCN1CCCC1)COC(=O)CC12CC3CC(CC(C3)C1)C2] |
| 159 | 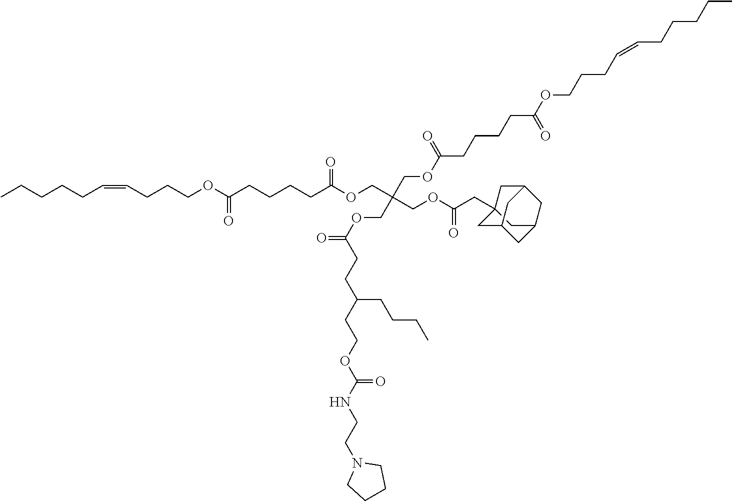 [SMILES: CCCCC/C=C\CCCOC(=O)CCCCC(=O)OCC(COC(=O)CCCCC(=O)OCCC/C=C\CCCCC)(COC(=O)CCC(CCCC)CCOC(=O)NCCN1CCCC1)COC(=O)CC12CC3CC(CC(C3)C1)C2] |
| 160 | 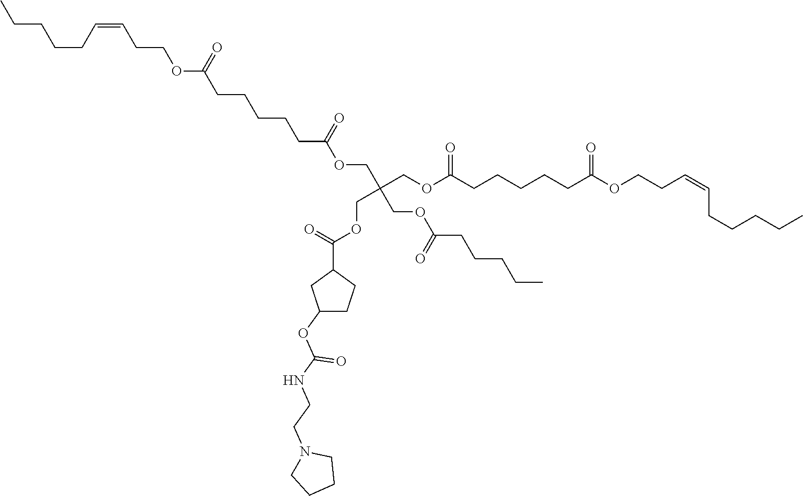 [SMILES: CCCCC/C=C\CCOC(=O)CCCCCC(=O)OCC(COC(=O)CCCCC)(COC(=O)CCCCCC(=O)OCC/C=C\CCCCC)COC(=O)C1CCC(OC(=O)NCCN2CCCC2)C1] |
| 161 | 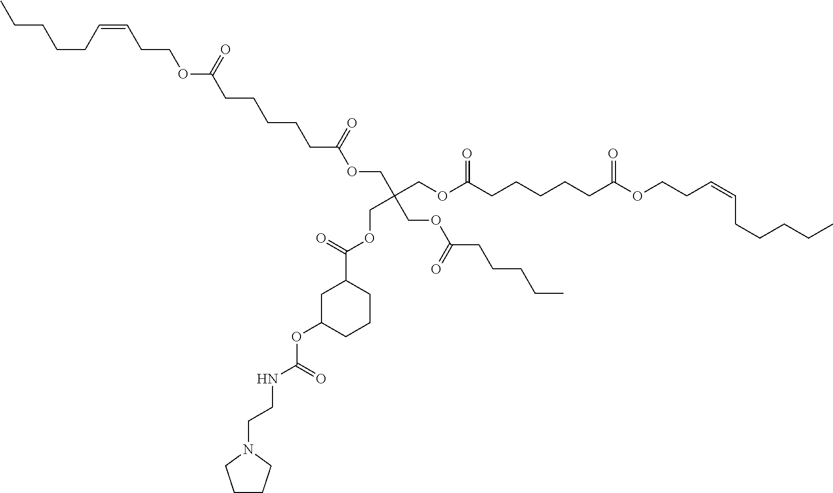 [SMILES: CCCCC/C=C\CCOC(=O)CCCCCC(=O)OCC(COC(=O)CCCCC)(COC(=O)CCCCCC(=O)OCC/C=C\CCCCC)COC(=O)C1CCCC(OC(=O)NCCN2CCCC2)C1] |
| 162 | 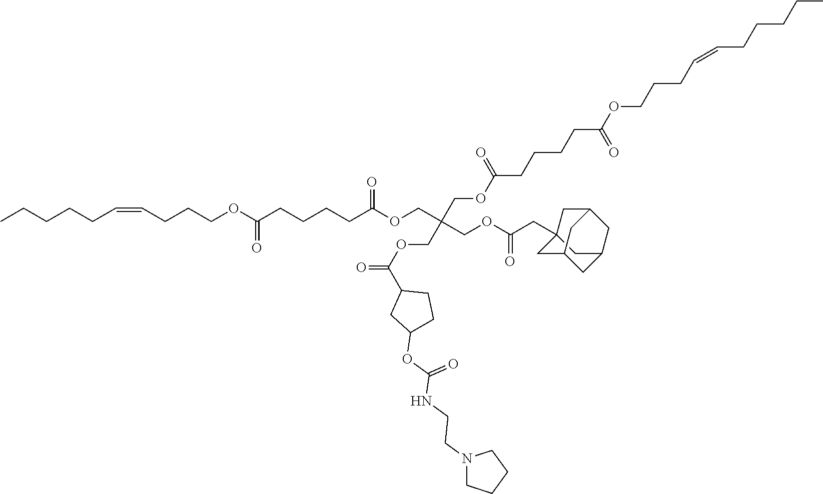 [SMILES: CCCCC/C=C\CCCOC(=O)CCCCC(=O)OCC(COC(=O)CCCCC(=O)OCCC/C=C\CCCCC)(COC(=O)CC12CC3CC(CC(C3)C1)C2)COC(=O)C1CCC(OC(=O)NCCN2CCCC2)C1] |
| 163 | 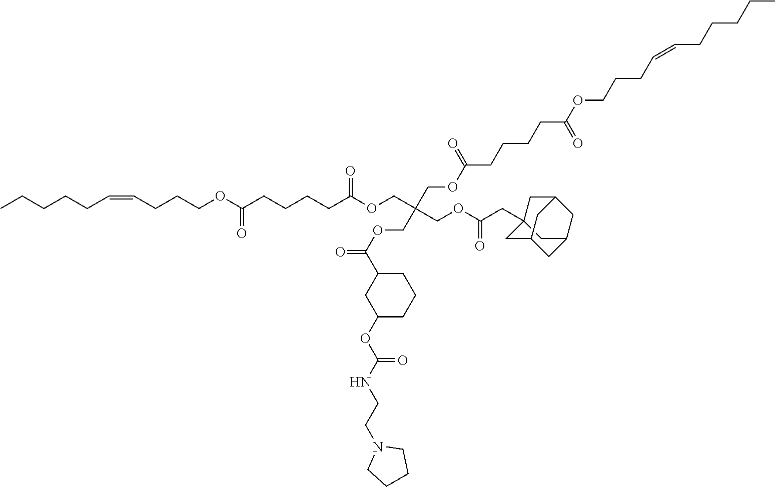 [SMILES: CCCCC/C=C\CCCOC(=O)CCCCC(=O)OCC(COC(=O)CCCCC(=O)OCCC/C=C\CCCCC)(COC(=O)CC12CC3CC(CC(C3)C1)C2)COC(=O)C1CCCC(OC(=O)NCCN2CCCC2)C1] |
| 164 | 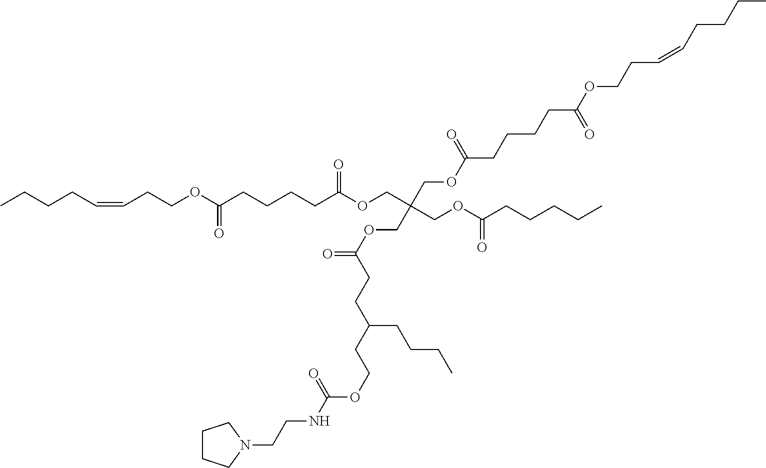 [SMILES: CCCC/C=C\CCOC(=O)CCCCC(=O)OCC(COC(=O)CCCCC)(COC(=O)CCCCC(=O)OCC/C=C\CCCC)COC(=O)CCC(CCCC)CCOC(=O)NCCN1CCCC1] |
| 165 | 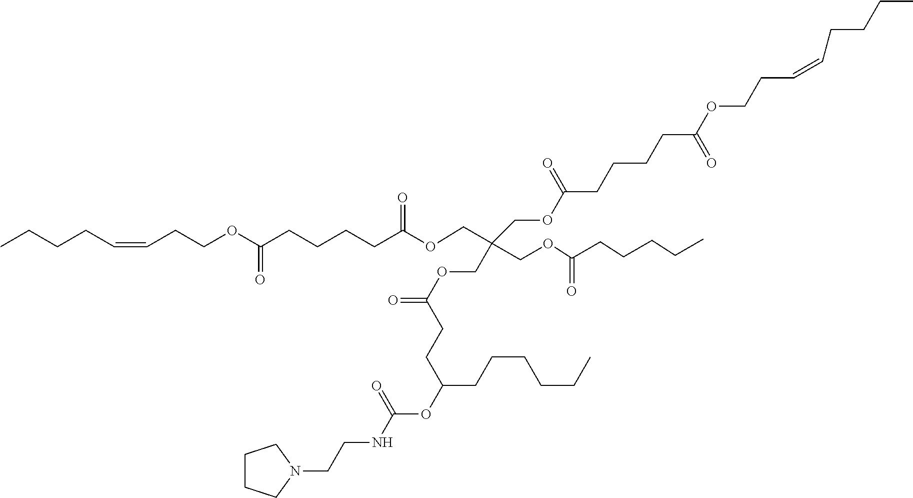 [SMILES: CCCC/C=C\CCOC(=O)CCCCC(=O)OCC(COC(=O)CCCCC)(COC(=O)CCCCC(=O)OCC/C=C\CCCC)COC(=O)CCC(CCCCCC)OC(=O)NCCN1CCCC1] |
| 166 | 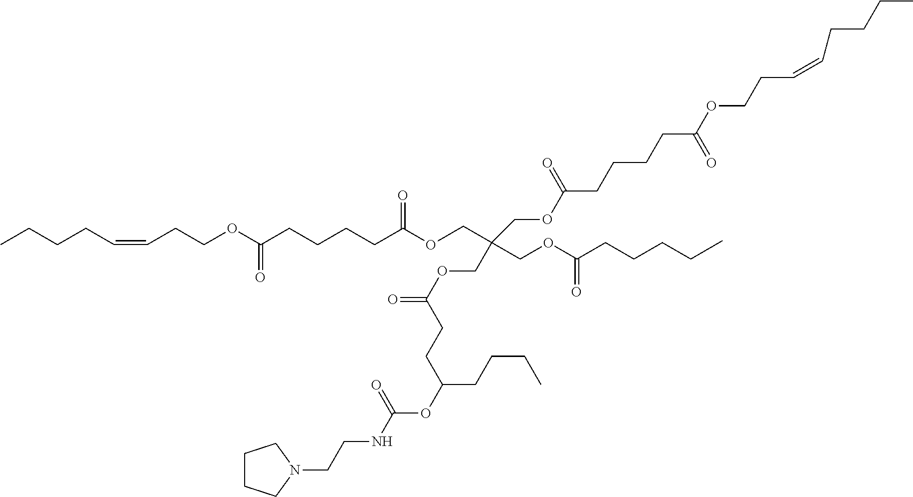 [SMILES: CCCC/C=C\CCOC(=O)CCCCC(=O)OCC(COC(=O)CCCCC)(COC(=O)CCCCC(=O)OCC/C=C\CCCC)COC(=O)CCC(CCCC)OC(=O)NCCN1CCCC1] |
| 167 | 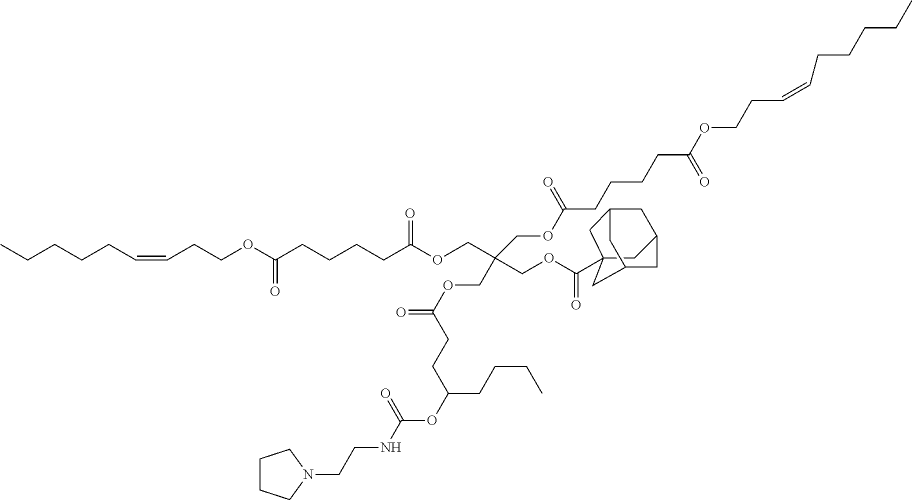 [SMILES: CCCCC/C=C\CCOC(=O)CCCCC(=O)OCC(COC(=O)CCCCC(=O)OCC/C=C\CCCCC)(COC(=O)CCC(CCCC)OC(=O)NCCN1CCCC1)COC(=O)C12CC3CC(CC(C3)C1)C2] |
| 168 | 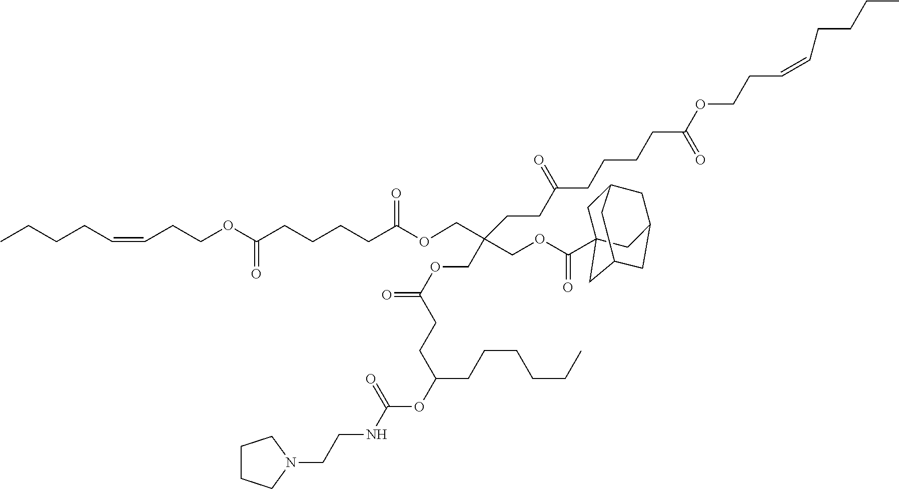 [SMILES: CCCC/C=C\CCOC(=O)CCCCC(=O)CCC(COC(=O)CCCCC(=O)OCC/C=C\CCCC)(COC(=O)CCC(CCCCCC)OC(=O)NCCN1CCCC1)COC(=O)C12CC3CC(CC(C3)C1)C2] |
| 169 | 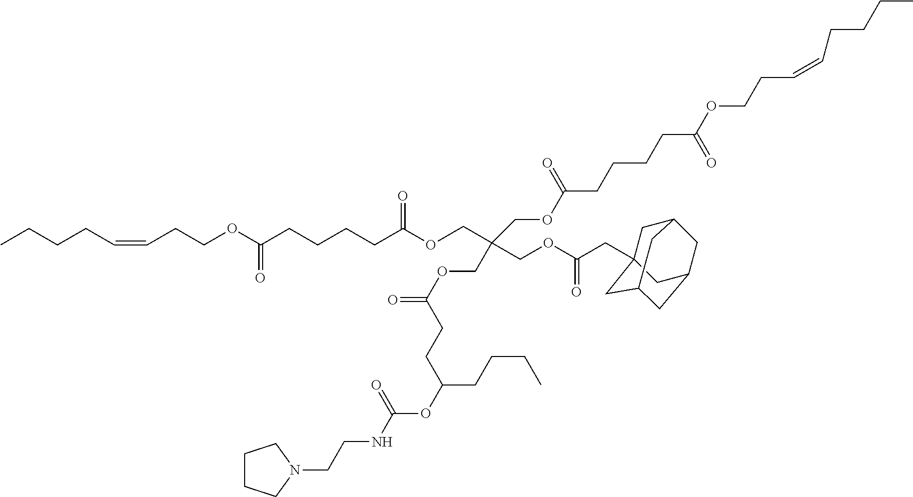 [SMILES: CCCC/C=C\CCOC(=O)CCCCC(=O)OCC(COC(=O)CCCCC(=O)OCC/C=C\CCCC)(COC(=O)CCC(CCCC)OC(=O)NCCN1CCCC1)COC(=O)CC12CC3CC(CC(C3)C1)C2] |
| 170 | 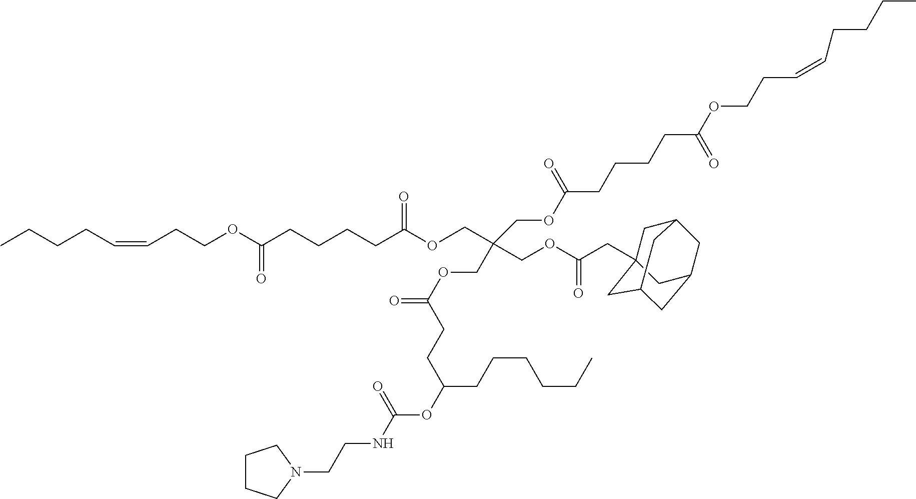 [SMILES: CCCC/C=C\CCOC(=O)CCCCC(=O)OCC(COC(=O)CCCCC(=O)OCC/C=C\CCCC)(COC(=O)CCC(CCCCCC)OC(=O)NCCN1CCCC1)COC(=O)CC12CC3CC(CC(C3)C1)C2] |
| 171 | 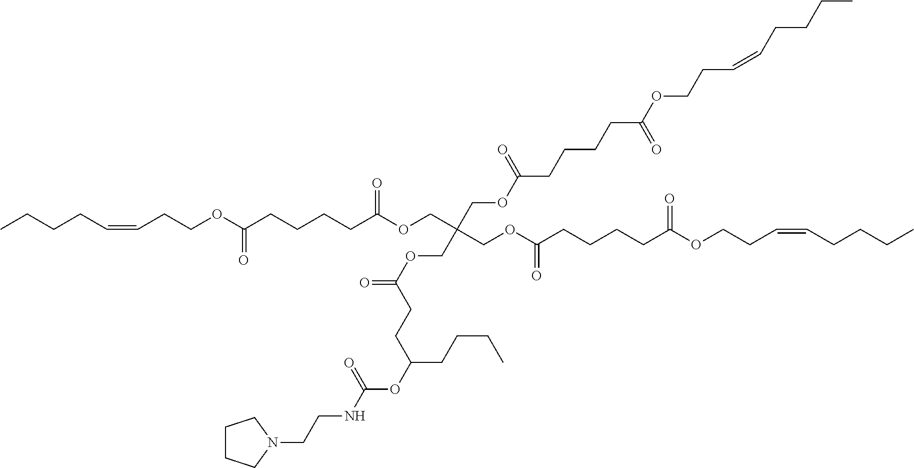 [SMILES: CCCC/C=C\CCOC(=O)CCCCC(=O)OCC(COC(=O)CCCCC(=O)OCC/C=C\CCCC)(COC(=O)CCCCC(=O)OCC/C=C\CCCC)COC(=O)CCC(CCCC)OC(=O)NCCN1CCCC1] |
| 172 | 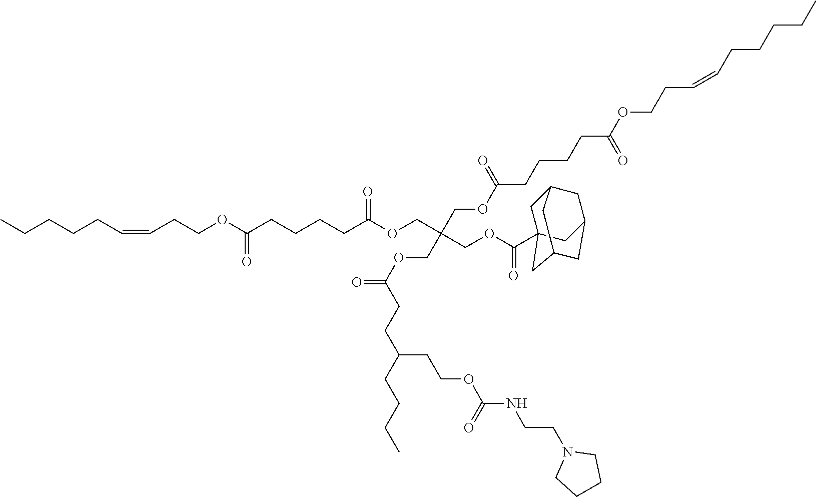 [SMILES: CCCCC/C=C\CCOC(=O)CCCCC(=O)OCC(COC(=O)CCCCC(=O)OCC/C=C\CCCCC)(COC(=O)CCC(CCCC)CCOC(=O)NCCN1CCCC1)COC(=O)C12CC3CC(CC(C3)C1)C2] |
| 173 | 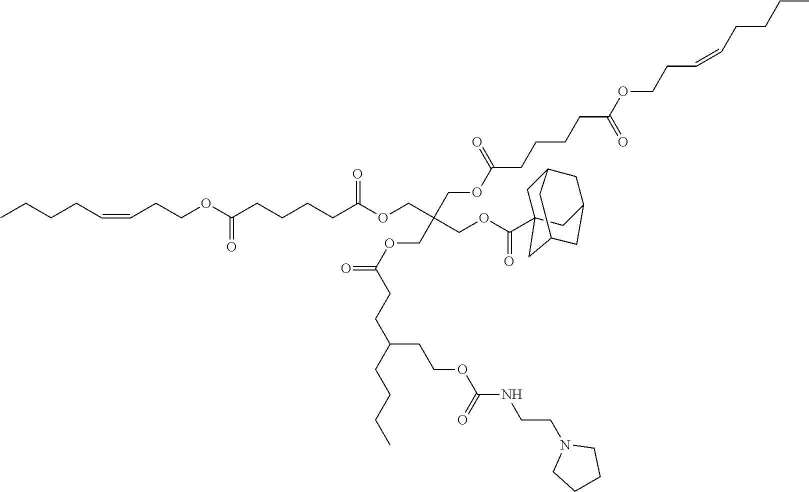 [SMILES: CCCC/C=C\CCOC(=O)CCCCC(=O)OCC(COC(=O)CCCCC(=O)OCC/C=C\CCCC)(COC(=O)CCC(CCCC)CCOC(=O)NCCN1CCCC1)COC(=O)C12CC3CC(CC(C3)C1)C2] |
| 174 | 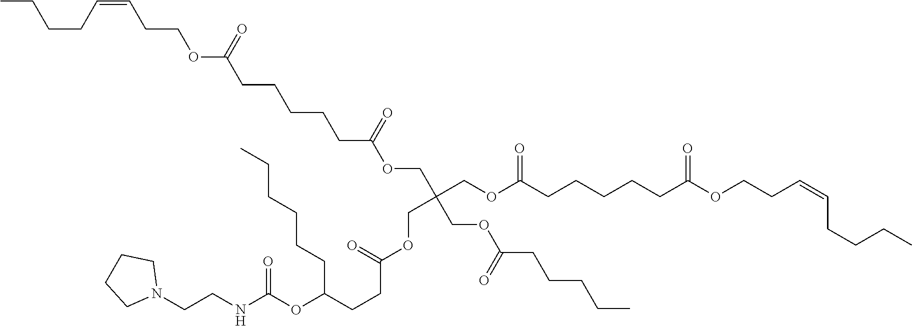 [SMILES: CCCC/C=C\CCOC(=O)CCCCCC(=O)OCC(COC(=O)CCCCC)(COC(=O)CCCCCC(=O)OCC/C=C\CCCC)COC(=O)CCC(CCCCCC)OC(=O)NCCN1CCCC1] |
| 175 | 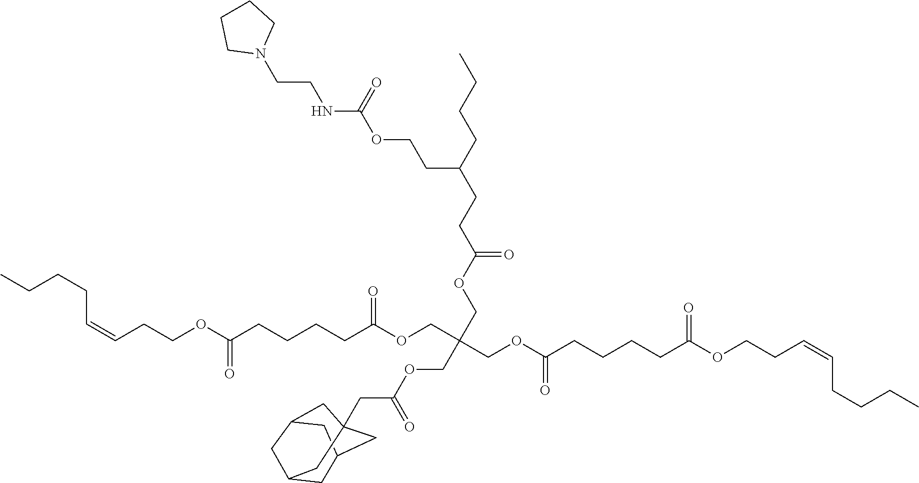 [SMILES: CCCC/C=C\CCOC(=O)CCCCC(=O)OCC(COC(=O)CCCCC(=O)OCC/C=C\CCCC)(COC(=O)CCC(CCCC)CCOC(=O)NCCN1CCCC1)COC(=O)CC12CC3CC(CC(C3)C1)C2] |
| 176 | 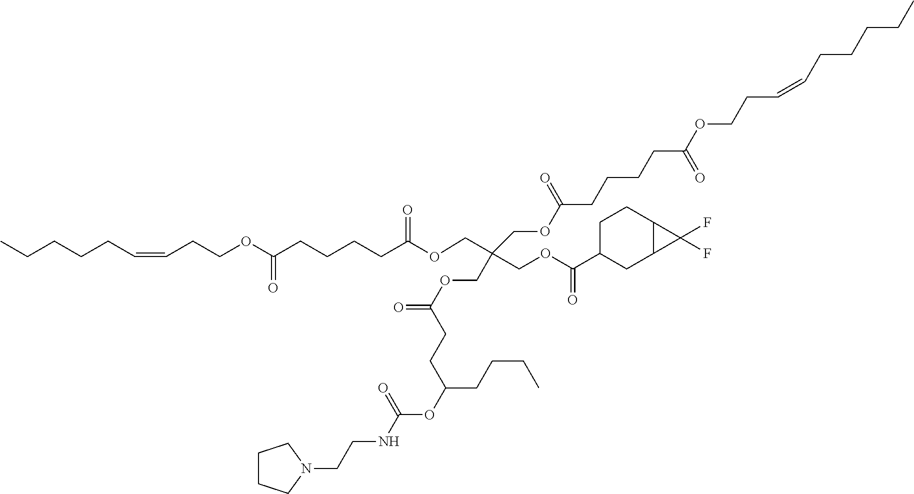 [SMILES: CCCCC/C=C\CCOC(=O)CCCCC(=O)OCC(COC(=O)CCCCC(=O)OCC/C=C\CCCCC)(COC(=O)CCC(CCCC)OC(=O)NCCN1CCCC1)COC(=O)C1CCC2C(C1)C2(F)F] |
| 177 | 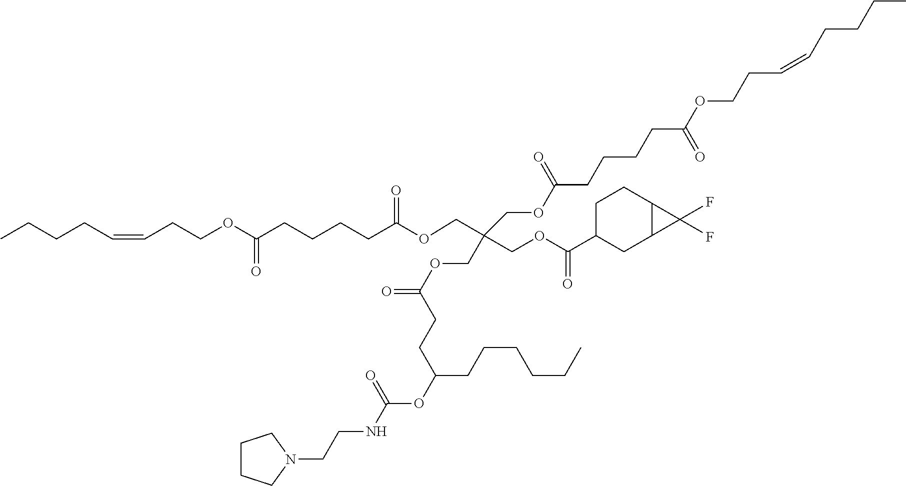 [SMILES: CCCC/C=C\CCOC(=O)CCCCC(=O)OCC(COC(=O)CCCCC(=O)OCC/C=C\CCCC)(COC(=O)CCC(CCCCCC)OC(=O)NCCN1CCCC1)COC(=O)C1CCC2C(C1)C2(F)F] |
| 178 | 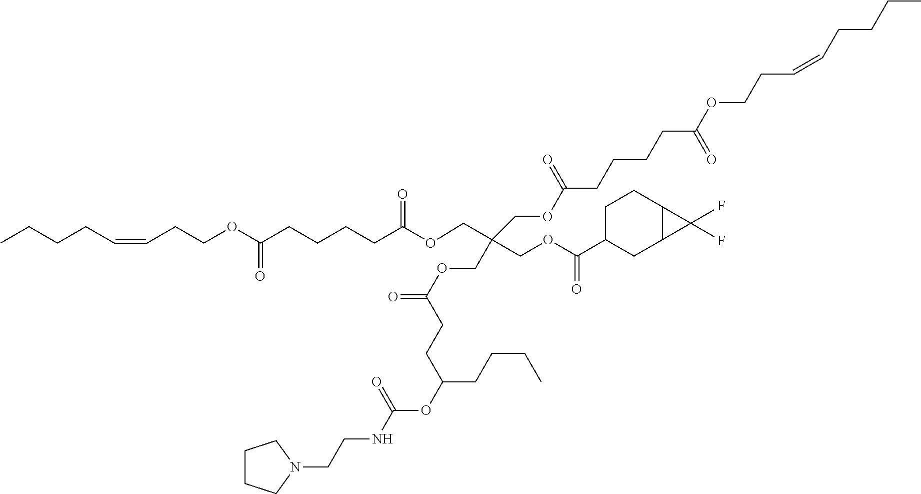 [SMILES: CCCC/C=C\CCOC(=O)CCCCC(=O)OCC(COC(=O)CCCCC(=O)OCC/C=C\CCCC)(COC(=O)CCC(CCCC)OC(=O)NCCN1CCCC1)COC(=O)C1CCC2C(C1)C2(F)F] |
| 179 | 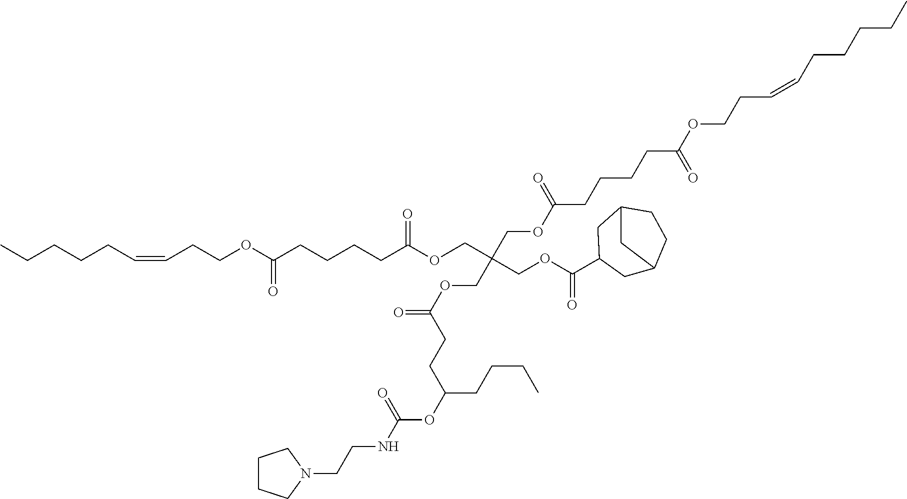 [SMILES: CCCCC/C=C\CCOC(=O)CCCCC(=O)OCC(COC(=O)CCCCC(=O)OCC/C=C\CCCCC)(COC(=O)CCC(CCCC)OC(=O)NCCN1CCCC1)COC(=O)C1CC2CCC(C2)C1] |
| 180 | 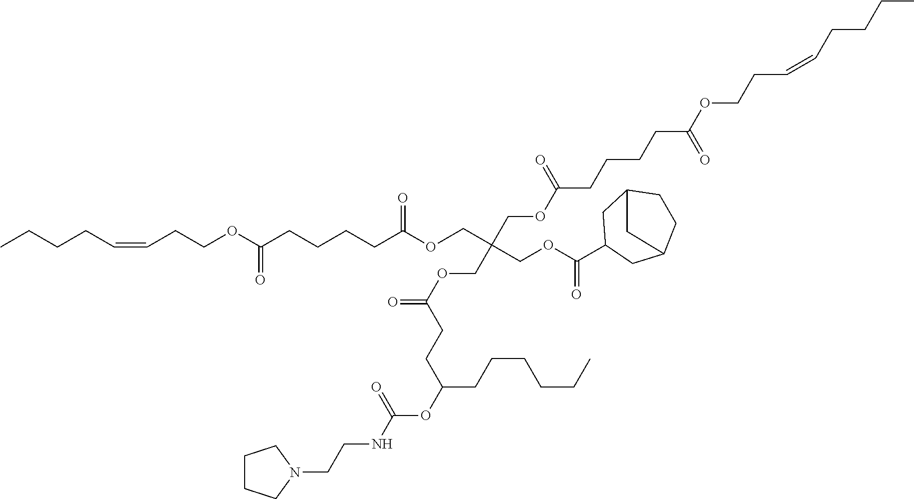 [SMILES: CCCC/C=C\CCOC(=O)CCCCC(=O)OCC(COC(=O)CCCCC(=O)OCC/C=C\CCCC)(COC(=O)CCC(CCCCCC)OC(=O)NCCN1CCCC1)COC(=O)C1CC2CCC(C2)C1] |
| 181 | 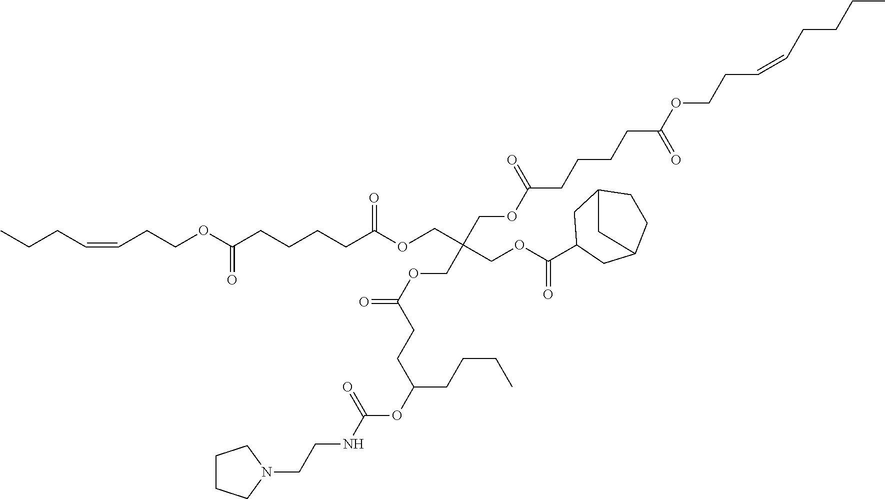 [SMILES: CCC/C=C\CCOC(=O)CCCCC(=O)OCC(COC(=O)CCCCC(=O)OCC/C=C\CCCC)(COC(=O)CCC(CCCC)OC(=O)NCCN1CCCC1)COC(=O)C1CC2CCC(C2)C1] |
| 182 | 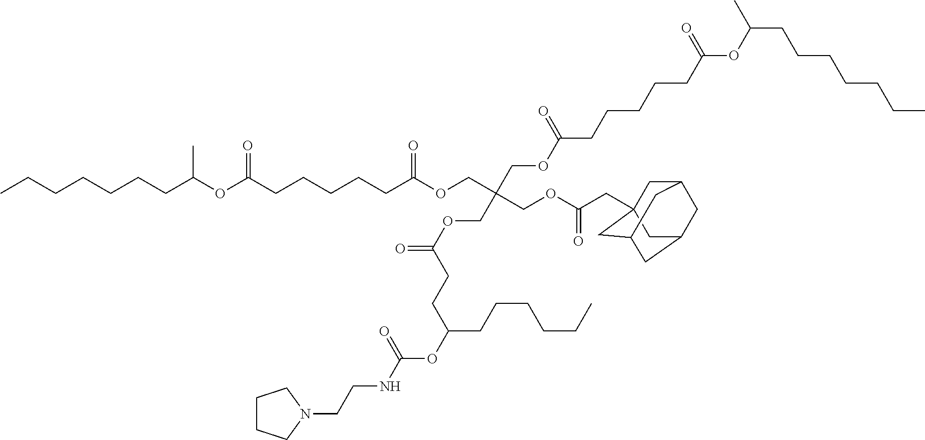 [SMILES: CCCCCCCC(C)OC(=O)CCCCCC(=O)OCC(COC(=O)CCCCCC(=O)OC(C)CCCCCCC)(COC(=O)CCC(CCCCCC)OC(=O)NCCN1CCCC1)COC(=O)CC12CC3CC(CC(C3)C1)C2] |
| 183 | 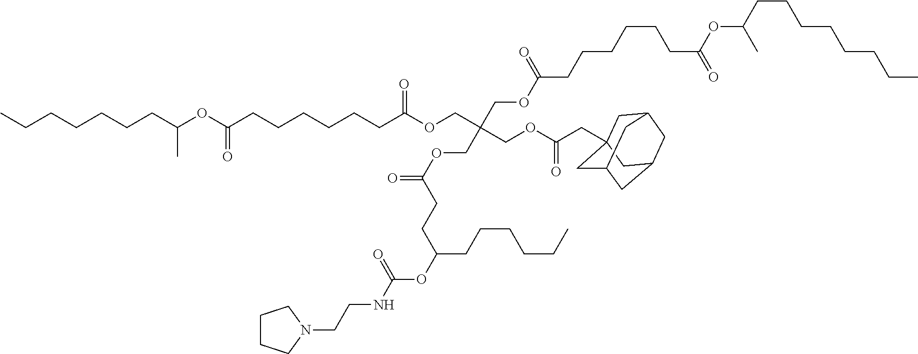 [SMILES: CCCCCCCCC(C)OC(=O)CCCCCCC(=O)OCC(COC(=O)CCCCCCC(=O)OC(C)CCCCCCC)(COC(=O)CCC(CCCCCC)OC(=O)NCCN1CCCC1)COC(=O)CC12CC3CC(CC(C3)C1)C2] |
| 184 | 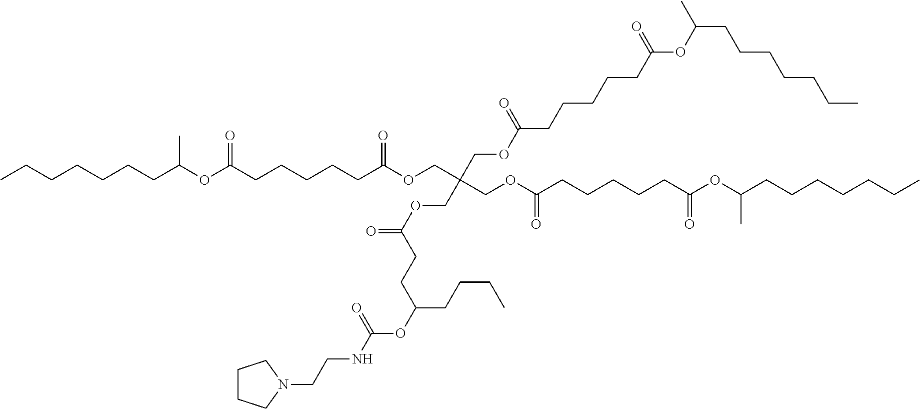 [SMILES: CCCCCCCC(C)OC(=O)CCCCCC(=O)OCC(COC(=O)CCCCCC(=O)OC(C)CCCCCCC)(COC(=O)CCCCCC(=O)OC(C)CCCCCCC)COC(=O)CCC(CCCC)OC(=O)NCCN1CCCC1] |
| 185 | 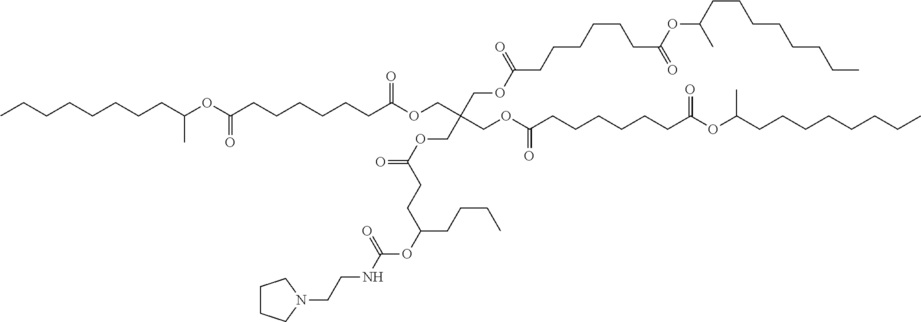 [SMILES: CCCCCCCCC(C)OC(=O)CCCCCCC(=O)OCC(COC(=O)CCCCCCC(=O)OC(C)CCCCCCCC)(COC(=O)CCCCCCC(=O)OC(C)CCCCCCCC)COC(=O)CCC(CCCC)OC(=O)NCCN1CCCC1] |
| 186 | 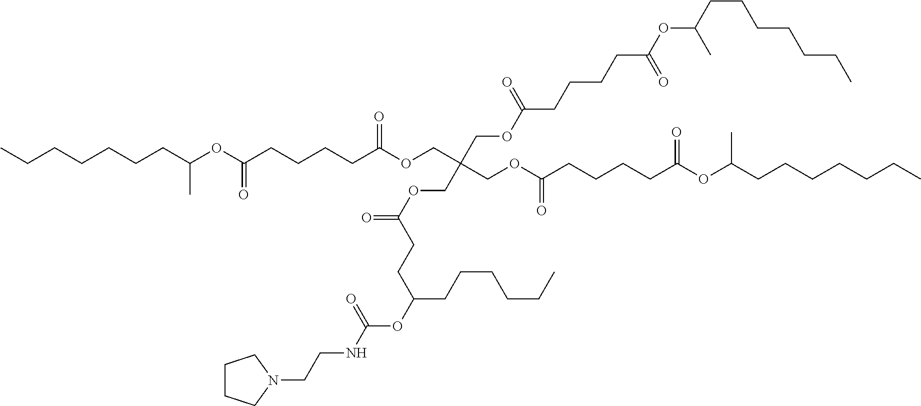 [SMILES: CCCCCCCC(C)OC(=O)CCCCC(=O)OCC(COC(=O)CCCCC(=O)OC(C)CCCCCCC)(COC(=O)CCCCC(=O)OC(C)CCCCCCC)COC(=O)CCC(CCCCCC)OC(=O)NCCN1CCCC1] |
| 187 | 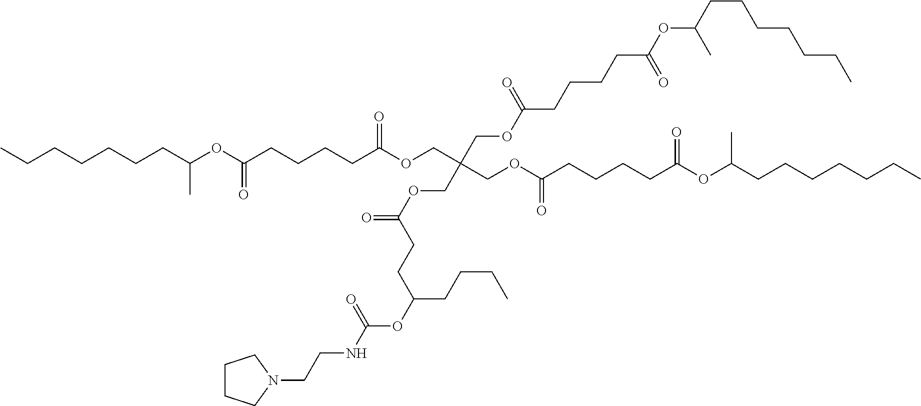 [SMILES: CCCCCCCC(C)OC(=O)CCCCC(=O)OCC(COC(=O)CCCCC(=O)OC(C)CCCCCCC)(COC(=O)CCCCC(=O)OC(C)CCCCCCC)COC(=O)CCC(CCCC)OC(=O)NCCN1CCCC1] |
| 188 | 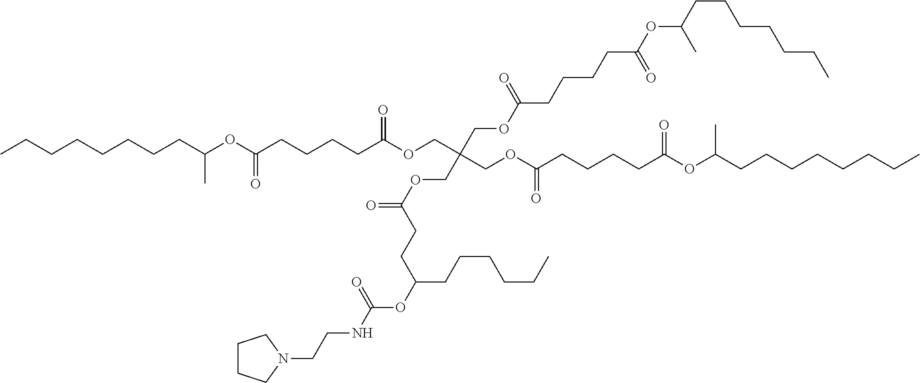 [SMILES: CCCCCCCCC(C)OC(=O)CCCCC(=O)OCC(COC(=O)CCCCC(=O)OC(C)CCCCCCC)(COC(=O)CCCCC(=O)OC(C)CCCCCCCC)COC(=O)CCC(CCCCCC)OC(=O)NCCN1CCCC1] |
| 189 | 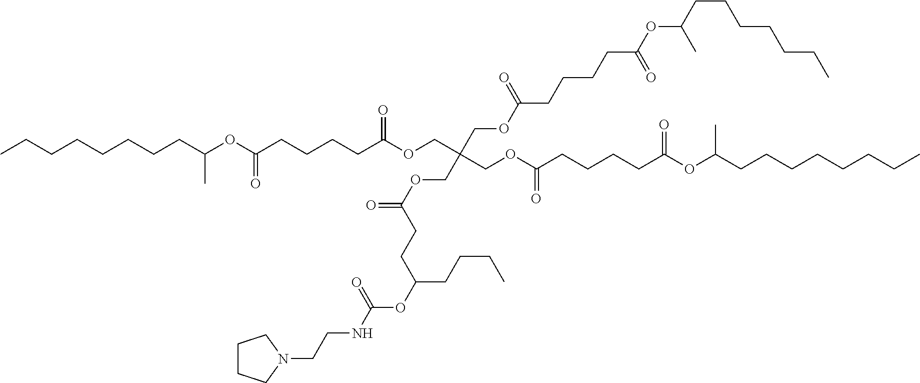 [SMILES: CCCCCCCCC(C)OC(=O)CCCCC(=O)OCC(COC(=O)CCCCC(=O)OC(C)CCCCCCC)(COC(=O)CCCCC(=O)OC(C)CCCCCCCC)COC(=O)CCC(CCCC)OC(=O)NCCN1CCCC1] |
| 190 | 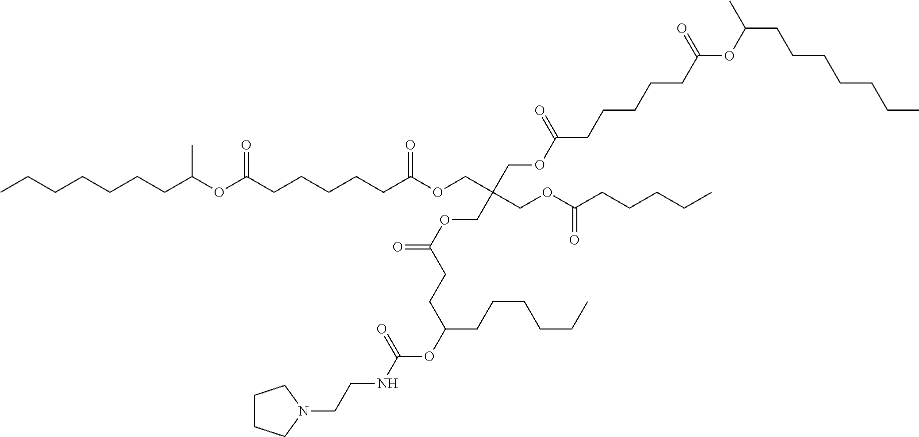 [SMILES: CCCCCCCC(C)OC(=O)CCCCCC(=O)OCC(COC(=O)CCCCC)(COC(=O)CCCCCC(=O)OC(C)CCCCCCC)COC(=O)CCC(CCCCCC)OC(=O)NCCN1CCCC1] |
| 191 | 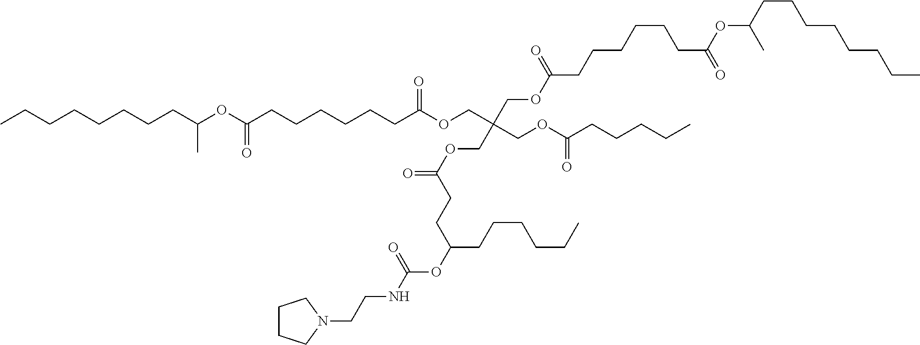 [SMILES: CCCCCCCCC(C)OC(=O)CCCCCCC(=O)OCC(COC(=O)CCCCC)(COC(=O)CCCCCCC(=O)OC(C)CCCCCCCC)COC(=O)CCC(CCCCCC)OC(=O)NCCN1CCCC1] |
| 192 | 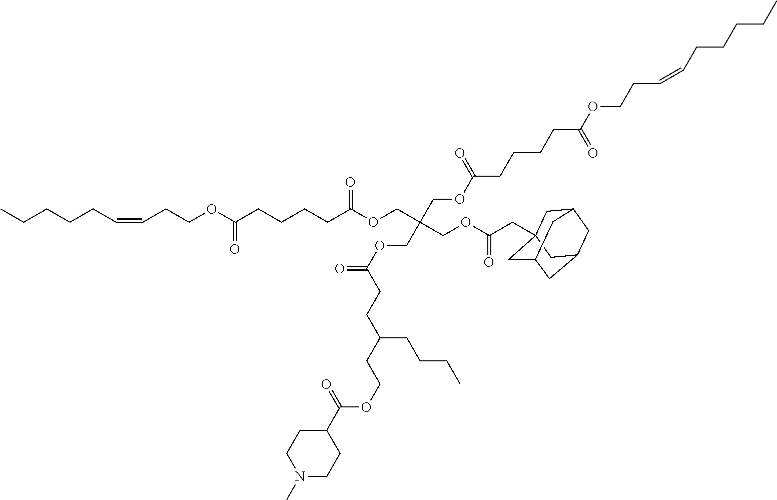 [SMILES: CCCCC/C=C\CCOC(=O)CCCCC(=O)OCC(COC(=O)CCCCC(=O)OCC/C=C\CCCCC)(COC(=O)CCC(CCCC)CCOC(=O)C1CCN(C)CC1)COC(=O)CC12CC3CC(CC(C3)C1)C2] |
| 193 | 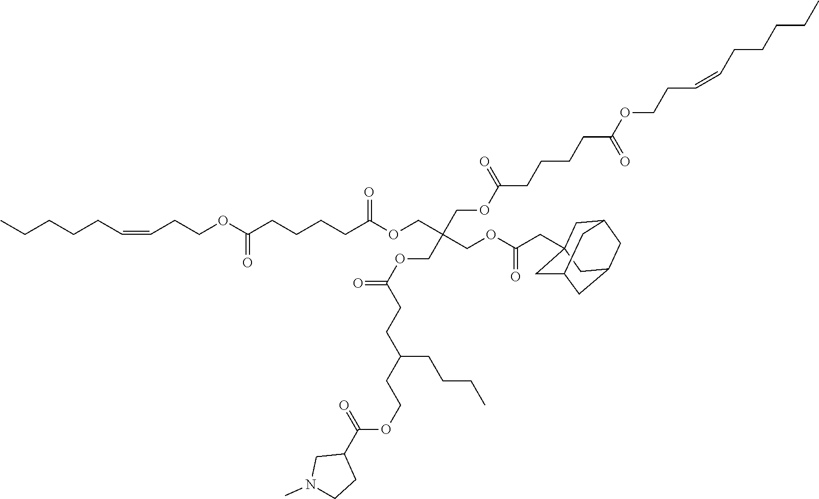 [SMILES: CCCCC/C=C\CCOC(=O)CCCCC(=O)OCC(COC(=O)CCCCC(=O)OCC/C=C\CCCCC)(COC(=O)CCC(CCCC)CCOC(=O)C1CCN(C)C1)COC(=O)CC12CC3CC(CC(C3)C1)C2] |
| 194 | 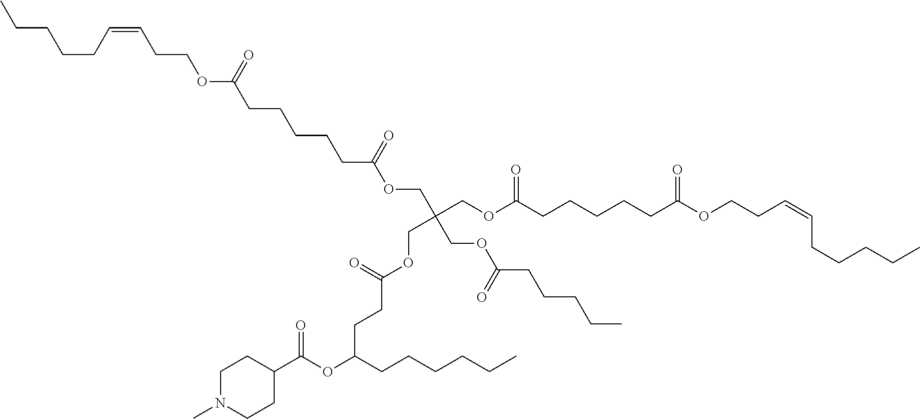 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCCCCC)OC(=O)C1CCN(C)CC1] |
| 195 | 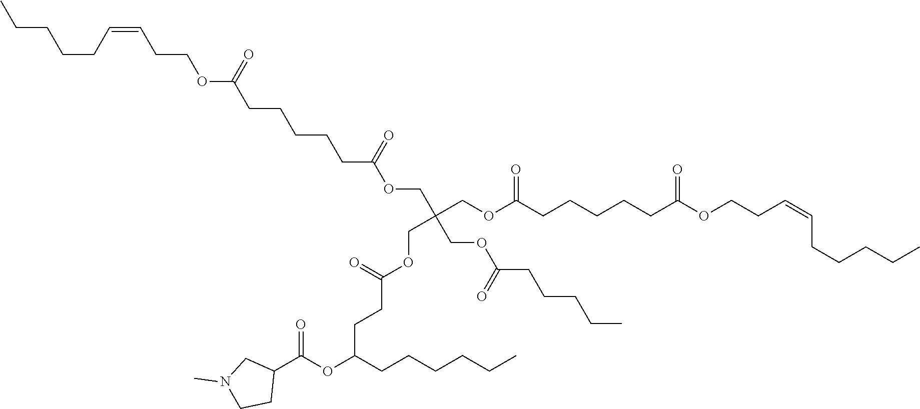 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCCCCC)OC(=O)C1CCN(C)C1] |
| 196 | 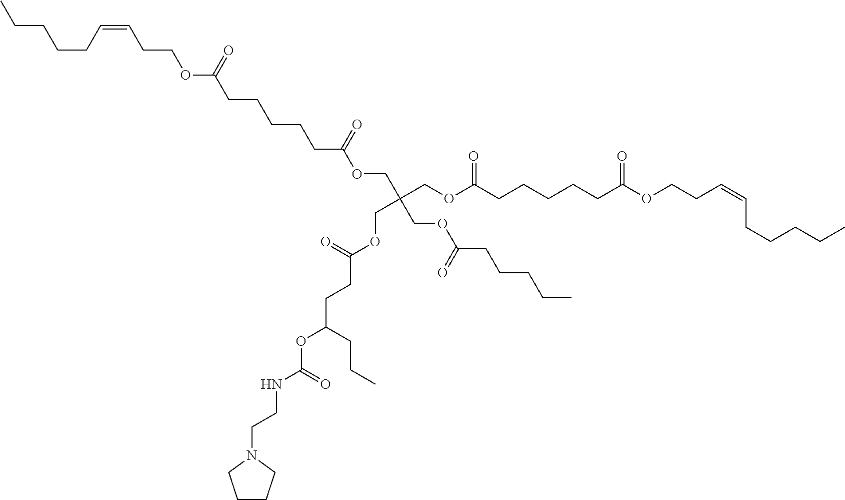 [SMILES: CCCCC/C=C\CCOC(=O)CCCCCC(=O)OCC(COC(=O)CCCCC)(COC(=O)CCCCCC(=O)OCC/C=C\CCCCC)COC(=O)CCC(CCC)OC(=O)NCCN1CCCC1] |
| 197 | 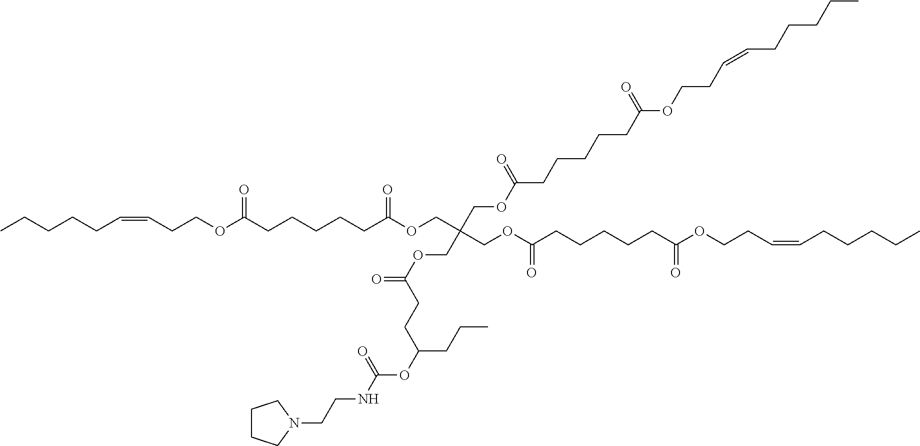 [SMILES: CCCCC/C=C\CCOC(=O)CCCCCC(=O)OCC(COC(=O)CCCCCC(=O)OCC/C=C\CCCCC)(COC(=O)CCCCCC(=O)OCC/C=C\CCCCC)COC(=O)CCC(CCC)OC(=O)NCCN1CCCC1] |
| 198 | 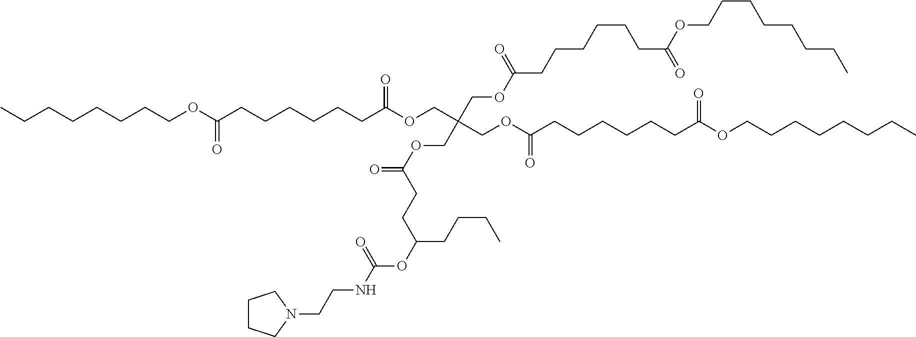 [SMILES: CCCCCCCCOC(=O)CCCCCCC(=O)OCC(COC(=O)CCCCCCC(=O)OCCCCCCCC)(COC(=O)CCCCCCC(=O)OCCCCCCCC)COC(=O)CCC(CCCC)OC(=O)NCCN1CCCC1] |
| 199 | 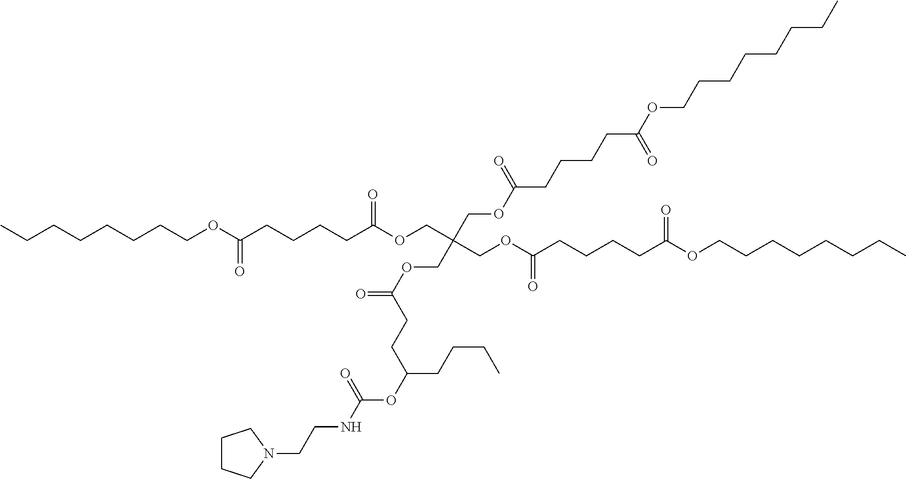 [SMILES: CCCCCCCCOC(=O)CCCCC(=O)OCC(COC(=O)CCCCC(=O)OCCCCCCCC)(COC(=O)CCCCC(=O)OCCCCCCCC)COC(=O)CCC(CCCC)OC(=O)NCCN1CCCC1] |
| 200 | 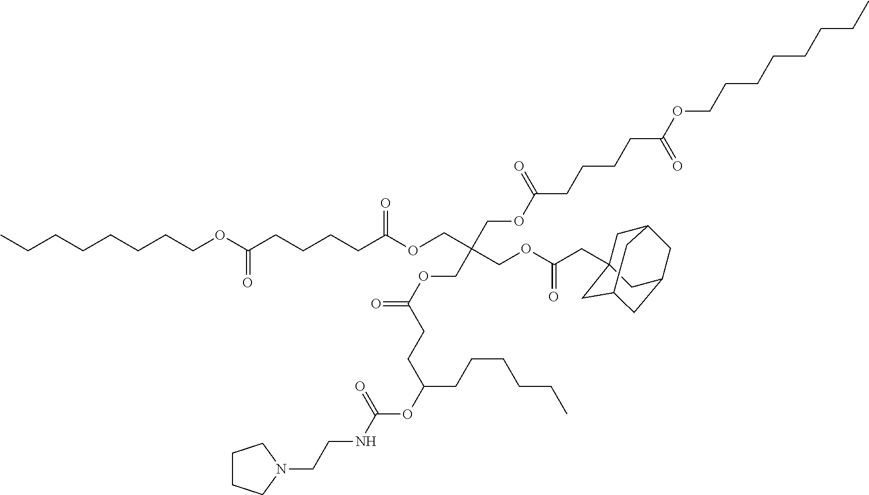 [SMILES: CCCCCCCCOC(=O)CCCCC(=O)OCC(COC(=O)CCCCC(=O)OCCCCCCCC)(COC(=O)CCC(CCCCCC)OC(=O)NCCN1CCCC1)COC(=O)CC12CC3CC(CC(C3)C1)C2] |
| 201 | 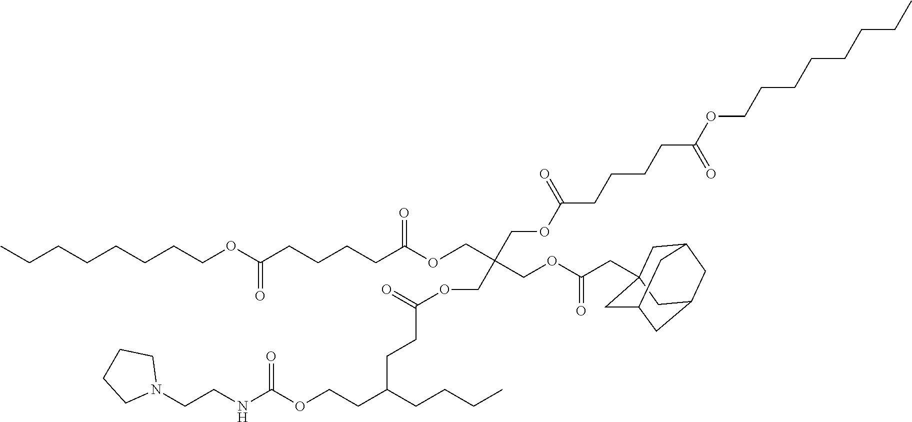 [SMILES: CCCCCCCCOC(=O)CCCCC(=O)OCC(COC(=O)CCCCC(=O)OCCCCCCCC)(COC(=O)CCC(CCCC)CCOC(=O)NCCN1CCCC1)COC(=O)CC12CC3CC(CC(C3)C1)C2] |
| 202 | 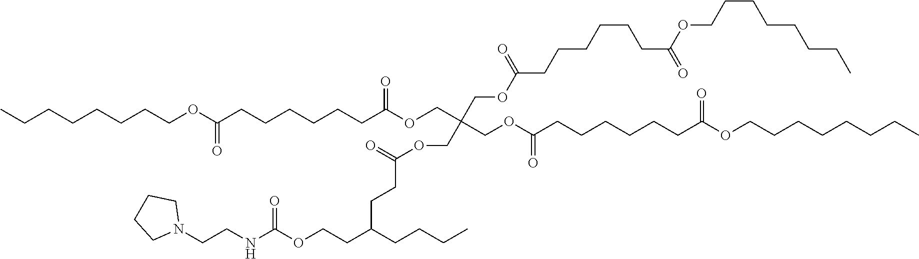 [SMILES: CCCCCCCCOC(=O)CCCCCCC(=O)OCC(COC(=O)CCCCCCC(=O)OCCCCCCCC)(COC(=O)CCCCCCC(=O)OCCCCCCCC)COC(=O)CCC(CCCC)CCOC(=O)NCCN1CCCC1] |
| 203 | 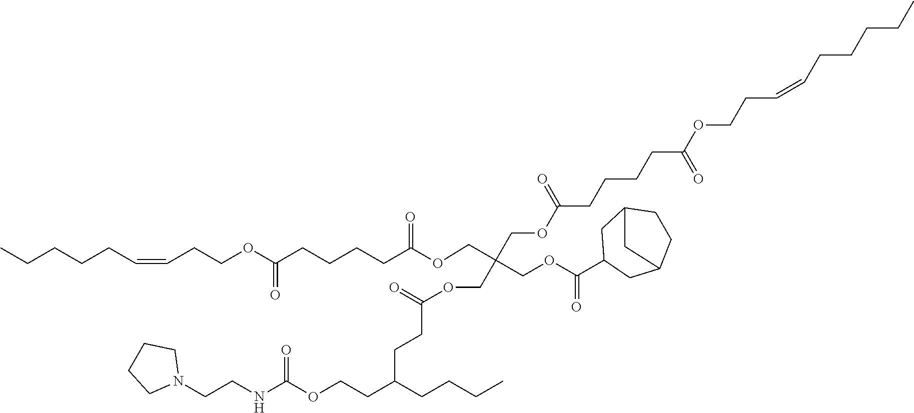 [SMILES: CCCCC/C=C\CCOC(=O)CCCCC(=O)OCC(COC(=O)CCCCC(=O)OCC/C=C\CCCCC)(COC(=O)CCC(CCCC)CCOC(=O)NCCN1CCCC1)COC(=O)C1CC2CCC(C2)C1] |
| 204 | 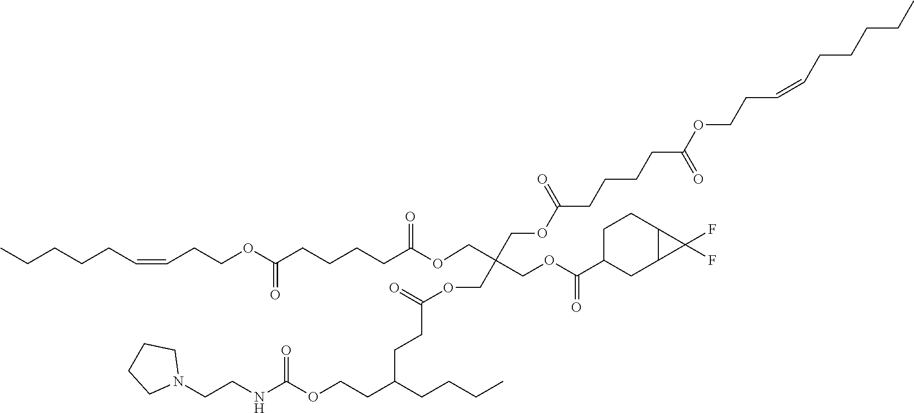 [SMILES: CCCCC/C=C\CCOC(=O)CCCCC(=O)OCC(COC(=O)CCCCC(=O)OCC/C=C\CCCCC)(COC(=O)CCC(CCCC)CCOC(=O)NCCN1CCCC1)COC(=O)C1CCC2C(C1)C2(F)F] |
| 205 | 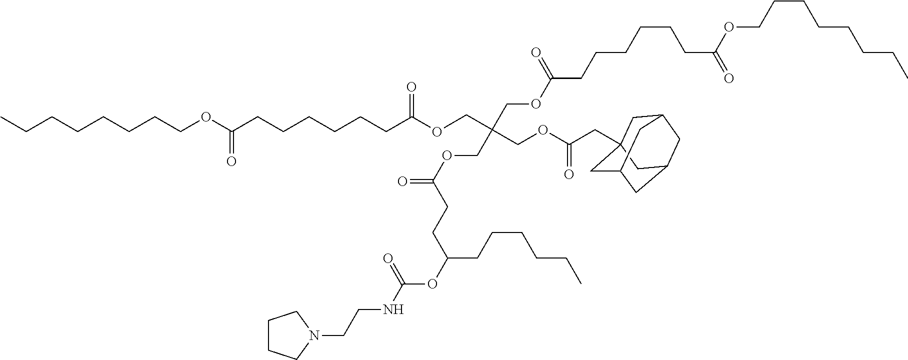 [SMILES: CCCCCCCCOC(=O)CCCCCCC(=O)OCC(COC(=O)CCCCCCC(=O)OCCCCCCCC)(COC(=O)CCC(CCCCCC)OC(=O)NCCN1CCCC1)COC(=O)CC12CC3CC(CC(C3)C1)C2] |
| 206 | 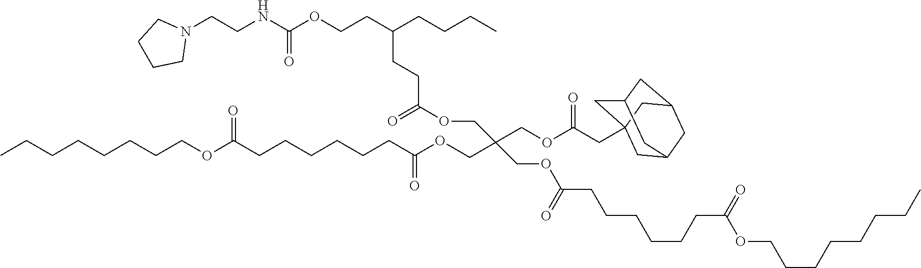 [SMILES: CCCCCCCCOC(=O)CCCCCCC(=O)OCC(COC(=O)CCCCCCC(=O)OCCCCCCCC)(COC(=O)CCC(CCCC)CCOC(=O)NCCN1CCCC1)COC(=O)CC12CC3CC(CC(C3)C1)C2] |
| 207 | 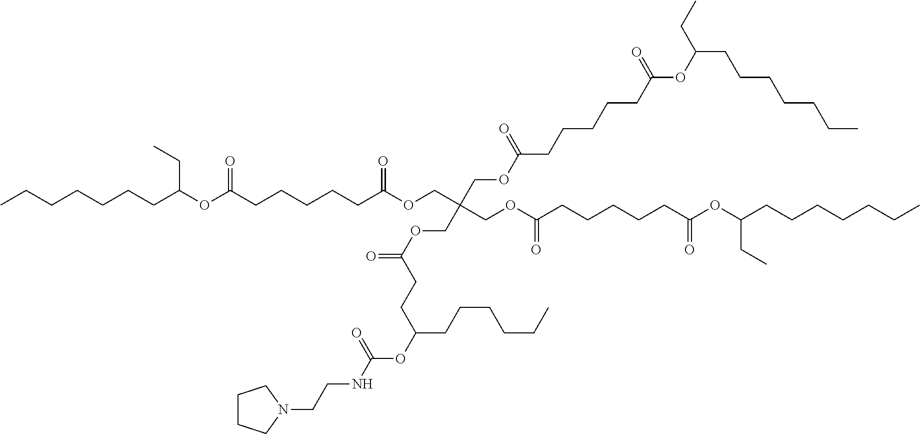 [SMILES: CCCCCCCC(CC)OC(=O)CCCCCC(=O)OCC(COC(=O)CCCCCC(=O)OC(CC)CCCCCCC)(COC(=O)CCCCCC(=O)OC(CC)CCCCCCC)COC(=O)CCC(CCCCCC)OC(=O)NCCN1CCCC1] |
| 208 | 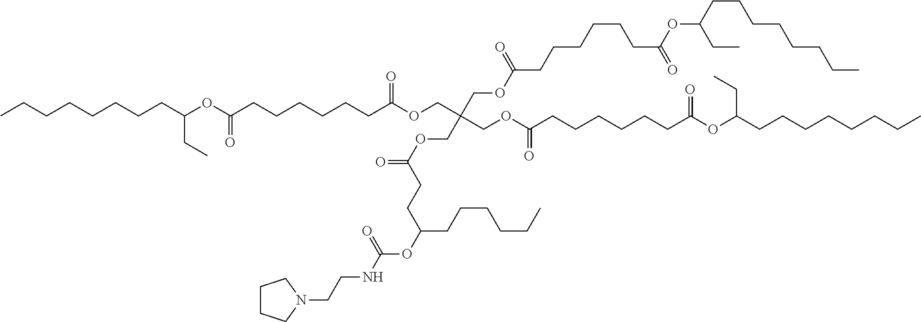 [SMILES: CCCCCCCCC(CC)OC(=O)CCCCCCC(=O)OCC(COC(=O)CCCCCCC(=O)OC(CC)CCCCCCCC)(COC(=O)CCCCCCC(=O)OC(CC)CCCCCCCC)COC(=O)CCC(CCCCCC)OC(=O)NCCN1CCCC1] |
| 209 | 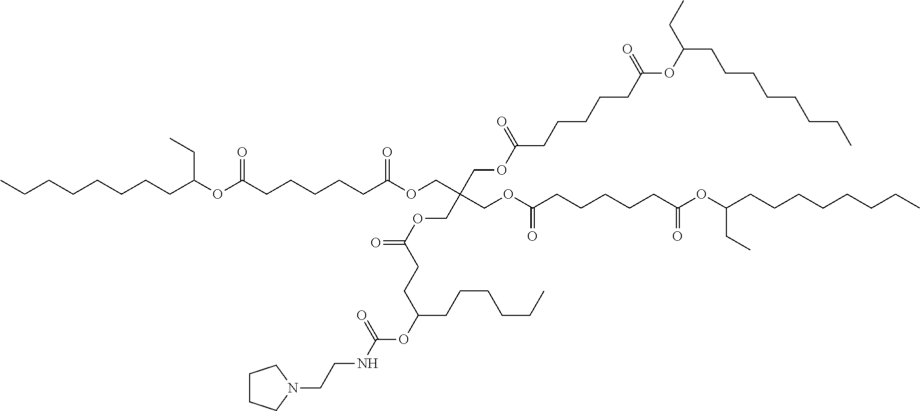 [SMILES: CCCCCCCCC(CC)OC(=O)CCCCCC(=O)OCC(COC(=O)CCCCCC(=O)OC(CC)CCCCCCCC)(COC(=O)CCCCCC(=O)OC(CC)CCCCCCCC)COC(=O)CCC(CCCCCC)OC(=O)NCCN1CCCC1] |

In certain embodiments, the lipid compound is any one of Compounds 1-209.

In certain embodiments, the lipid compound is any one of Compounds 7, 8, 10, 13, 14, 26, 33, 38, 39, 40, 48, 60, 61, 89, 103, or 109, or a pharmaceutically acceptable salt thereof.

In certain embodiments, the lipid compound is any one of Compounds 1-20, or a pharmaceutically acceptable salt thereof.

In certain embodiments, the lipid compound is Compound 14 or a pharmaceutically acceptable salt thereof.

In certain embodiments, the lipid compound is any one of Compounds 7, 8, 10, 13, or 18, or a pharmaceutically acceptable salt thereof.

In certain embodiments, the lipid compound is any one of Compounds 1-4 or 6-12, or a pharmaceutically acceptable salt thereof.

In certain embodiments, the lipid compound is any one of Compounds 17, 60, or 61, or a pharmaceutically acceptable salt thereof.

In certain embodiments, the lipid compound is any one of Compounds 50-58 or a pharmaceutically acceptable salt thereof.

In certain embodiments, the lipid compound is any one of Compounds 31, 41-43, or 47, or a pharmaceutically acceptable salt thereof.

In certain embodiments, the lipid compound is any one of Compounds 24, 25, 28, 29, 33-36, 38, or 40, or a pharmaceutically acceptable salt thereof.

In certain embodiments, the lipid compound is any one of Compounds 62-34 or 66, or a pharmaceutically acceptable salt thereof.

In certain embodiments, the lipid compound is any one of Compounds 21, 23, 26, 27, 37, 39, or 44-46, or a pharmaceutically acceptable salt thereof.

In certain embodiments, the lipid compound is any one of Compounds 13-14, 67, 73, 81, 91, 93, 96, or 98-101, or a pharmaceutically acceptable salt thereof.

In certain embodiments, the lipid compound is any one of Compounds 14-15, 49-50, 59, or 102-103, or a pharmaceutically acceptable salt thereof.

In certain embodiments, the lipid compound is any one of Compounds 8, 14, 7, 10, or 60-61, or a pharmaceutically acceptable salt thereof.

In certain embodiments, the lipid compound of Formula (I) or Formula (II), or pharmaceutically acceptable salt thereof, is an ionizable lipid compound.

### II. Lipid Compositions

### Lipid Nanoparticles (LNP)

In certain embodiments, the present disclosure provides lipid compositions (e.g., a lipid nanoparticle (LNP)) comprising at least one lipid compound of Formula (I) or Formula (II), or a pharmaceutically acceptable salt thereof. In certain embodiments, the present disclosure provides lipid nanoparticles (LNPs) comprising at least one lipid compound of Formula (I) or Formula (II), or a pharmaceutically acceptable salt thereof. In certain embodiments, the lipid nanoparticles are microspherical vesicles (i.e., liposomes) that comprise liposomal lamellar phase lipid bilayers (unilamellar or multilamellar) which encompass an interlamellar space. In certain embodiments, the lipid nanoparticles are nanospherical vesicles that comprise liposomal lamellar phase lipid bilayers (unilamellar or multilamellar).

In certain embodiments, the lipid nanoparticle (LNP) comprises: (i) at least one lipid compound of Formula (I) or Formula (II), or a pharmaceutically acceptable salt thereof; (ii) at least one phospholipid (e.g., distearoylphosphatidylcholine (DSPC)); (iii) at least one helper lipid (e.g., cholesterol); and (iv) at least one PEG lipid. In certain embodiments, the LNP comprises: (i) at least one lipid compound of Formula (I) or Formula (II), or a pharmaceutically acceptable salt thereof; (ii) at least one phospholipid (e.g., distearoylphosphatidylcholine (DSPC)); (iii) at least one cholesterol; and (iv) at least one PEG lipid. In certain embodiments, the lipid nanoparticle (LNP) comprises one or more additional lipid components.

In certain embodiments, the lipid nanoparticle (LNP) comprises a lipid compound selected from any one of Compounds 1-209. In certain embodiments, the lipid nanoparticle (LNP) comprises a lipid compound selected from any one of Compounds 7, 8, 10, 13, 14, 26, 33, 38, 39, 40, 48, 60, 61, 89, 103, or 109, or a pharmaceutically acceptable salt thereof.

Phospholipids for use in a lipid composition (e.g., LNP) of the present disclosure can be neutral, uncharged, or zwitterionic phospholipids. Examples of phospholipids for use in a lipid composition include, but are not limited to: dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the phospholipid is distearoylphosphatidylcholine (DSPC).

Helper lipids for use in a lipid composition (e.g., LNP) of the present disclosure include steroids, sterols, and alkyl resorcinols. Examples of helper lipids for use in a lipid composition include, but are not limited to, cholesterol, cholesterol hemisuccinate, and 5-heptadecylresorcinol. In certain embodiments, the helper lipid is cholesterol.

PEG lipids for use in a lipid composition (e.g., LNP) of the present disclosure include compounds which comprise a lipid moiety bound to a PEG-based polymer moiety (i.e., PEG moiety). In certain embodiments, the lipid moiety of the PEG lipid is derived from diacylglycerol or diacylglycamide. In certain embodiments, the lipid moiety of the PEG lipid is derived from a dialkylglycerol or dialkylglycamide group. In certain embodiments, the dialkylglycerol or dialkylglycamide group has alkyl chain length from about C4 to about C40 saturated or unsaturated carbon atoms. In certain embodiments, the alkyl chain length is from about C10 to about C20. In certain embodiments, the dialkylglycerol or dialkylglycamide group comprises one or more functional groups (e.g., amide or ester). In certain embodiments, the dialkylglycerol or dialkylglycamide group comprises one or more substituted alkyl groups.

PEG moieties of the PEG lipid can include any polyethylene glycol (PEG) or other polyalkylene ether polymers, including optionally substituted linear or branched polymers of ethylene glycol or ethylene oxide. PEG moieties can have a molecular weight of: from about 130 to about 50,000 Da; from about 150 to about 25,000 Da; from about 150 to about 15,000 Da; from about 150 to about 10,000 Da; from about 150 to about 5,000 Da; from about 150 to about 4,000 Da; from about 150 to about 3,000 Da; from about 150 to about 2,500 Da; from about 150 to about 2,000 Da; from about 500 to about 3,000 Da; from about 500 to about 2,000 Da; from about 1,000 to about 3,000 Da; or from about 1,000 to about 2,000 Da. In certain embodiments, the PEG moiety comprises PEG2000 (having 2,000 Da).

In certain embodiments, the lipid composition (e.g., LNP) comprises about 40-70 mol % of at least one lipid compound of Formula (I) or Formula (II), or a pharmaceutically acceptable salt thereof. In certain embodiments, the lipid composition comprises about 20-80 mol % of a lipid compound of Formula (I) or Formula (II) or a pharmaceutically acceptable salt thereof. In certain embodiments, the lipid composition comprises about 45-65 mol % of a lipid compound of Formula (I) or Formula (II) or a pharmaceutically acceptable salt thereof. In certain embodiments, the lipid composition comprises about 45-50 mol % (e.g., about 47.5 mol %) of a lipid compound of Formula (I) or Formula (II) or a pharmaceutically acceptable salt thereof. In certain embodiments, the lipid composition comprises about 47.5-52.5 mol % (e.g., about 50 mol %) of a lipid compound of Formula (I) or Formula (II) or a pharmaceutically acceptable salt thereof. In certain embodiments, the lipid composition comprises about 50-55 mol % (e.g., about 52.5 mol %) of a lipid compound of Formula (I) or Formula (II) or a pharmaceutically acceptable salt thereof. In certain embodiments, the lipid composition comprises about 52.5-57.5 mol % (e.g., about 55 mol %) of a lipid compound of Formula (I) or Formula (II) or a pharmaceutically acceptable salt thereof. In certain embodiments, the lipid composition comprises about 55-60 mol % (e.g., about 57.5 mol %) of a lipid compound of Formula (I) or Formula (II) or a pharmaceutically acceptable salt thereof. In certain embodiments, the lipid composition comprises about 57.5-62.5 mol % (e.g., about 60 mol %) of a lipid compound of Formula (I) or Formula (II) or a pharmaceutically acceptable salt thereof. In certain embodiments, the lipid composition comprises about 60-65 mol % (e.g., about 62.5 mol %) of a lipid compound of Formula (I) or Formula (II) or a pharmaceutically acceptable salt thereof. In certain embodiments, the lipid composition comprises about 62.5-67.5 mol % (e.g., about 65 mol %) of a lipid compound of Formula (I) or Formula (II) or a pharmaceutically acceptable salt thereof.

In certain embodiments, the lipid composition (e.g., LNP) comprises about 7.5-40 mol % of a phospholipid (e.g., distearoylphosphatidylcholine (DSPC). In certain embodiments, the lipid composition (e.g., LNP) comprises about 7.5-12.5 mol % of a phospholipid (e.g., distearoylphosphatidylcholine (DSPC). In certain embodiments, the lipid composition comprises about 10 mol % of a phospholipid (e.g., distearoylphosphatidylcholine (DSPC).

In certain embodiments, the lipid composition (e.g., LNP) comprises about 6-50 mol % of a helper lipid (e.g., cholesterol). In certain embodiments, the lipid composition (e.g., LNP) comprises about 20-50 mol % of a helper lipid (e.g., cholesterol). In certain embodiments, the lipid composition (e.g., LNP) comprises about 6-45 mol % of a helper lipid (e.g., cholesterol). In certain embodiments, the lipid composition comprises about 25-45 mol % of a helper lipid (e.g., cholesterol). In certain embodiments, the lipid composition comprises about 26-29 mol % of a helper lipid (e.g., cholesterol). In certain embodiments, the lipid composition comprises about 27.5 mol % of a helper lipid (e.g., cholesterol). In certain embodiments, the lipid composition comprises about 37-40 mol % of a helper lipid (e.g., cholesterol). In certain embodiments, the lipid composition comprises about 38.5 mol % of a helper lipid (e.g., cholesterol). In certain embodiments, the lipid composition comprises about 37.5-40.5 mol % of a helper lipid (e.g., cholesterol). In certain embodiments, the lipid composition comprises about 39 mol % of a helper lipid (e.g., cholesterol). In certain embodiments, the lipid composition comprises about 38-42 mol % of a helper lipid (e.g., cholesterol). In certain embodiments, the lipid composition comprises about 40 mol % of a helper lipid (e.g., cholesterol).

In certain embodiments, the lipid composition (e.g., LNP) comprises about 1-4 mol % of a PEG lipid. In certain embodiments, the lipid composition comprises about 1-2 mol % (e.g., 1.5 mol %) of a PEG lipid. In certain embodiments, the lipid composition comprises about 1.5-2.5 mol % (e.g., 2 mol %) of a PEG lipid. In certain embodiments, the lipid composition comprises about 2-3 mol % (e.g., 2.5 mol %) of a PEG lipid. In certain embodiments, the lipid composition comprises about 2.5-3.5 mol % (e.g., 3 mol %) of a PEG lipid. In certain embodiments, the lipid composition comprises about 3-4 mol % (e.g., 3.5 mol %) of a PEG lipid.

In certain embodiments, the lipid composition (e.g., LNP) comprises: (i) about 20-80 mol % of a lipid compound of Formula (I) or Formula (II) or a pharmaceutically acceptable salt thereof; (ii) about 7.5-40 mol % of a phospholipid (e.g., distearoylphosphatidylcholine (DSPC); (iii) about 6-45 mol % of a helper lipid (e.g., cholesterol); and (iv) about 1-4 mol % of a PEG lipid. In certain embodiments, the lipid composition (e.g., LNP) comprises: (i) about 40-70 mol % of a lipid compound of Formula (I) or Formula (II) or a pharmaceutically acceptable salt thereof; (ii) about 7.5-12.5 mol % of a phospholipid (e.g., distearoylphosphatidylcholine (DSPC); (iii) about 20-50 mol % of a helper lipid (e.g., cholesterol); and (iv) about 1-4 mol % of a PEG lipid. In certain embodiments, the lipid composition comprises: (i) about 45-65 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 25-45 mol % of a helper lipid (e.g., cholesterol); and (iv) about 1-4 mol % of a PEG lipid.

In certain embodiments, the lipid composition (e.g., LNP) comprises: (i) about 45-50 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 38-42 mol % of a helper lipid (e.g., cholesterol); and (iv) about 2-3 mol % of a PEG lipid. In certain embodiments, the lipid composition comprises: (i) about 47.5 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 40 mol % of a helper lipid (e.g., cholesterol); and (iv) about 2.5 mol % of a PEG lipid.

In certain embodiments, the lipid composition (e.g., LNP) comprises: (i) about 47.5-52.5 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 37-40 mol % of a helper lipid (e.g., cholesterol); and (iv) about 1-2 mol % of a PEG lipid. In certain embodiments, the lipid composition comprises: (i) about 50 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 38.5 mol % of a helper lipid (e.g., cholesterol); and (iv) about 1.5 mol % of a PEG lipid.

In certain embodiments, the lipid composition (e.g., LNP) comprises: (i) about 57.5-62.5 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 26-29 mol % of a helper lipid (e.g., cholesterol); and (iv) about 2-3 mol % of a PEG lipid. In certain embodiments, the lipid composition comprises: (i) about 60 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 27.5 mol % of a helper lipid (e.g., cholesterol); and (iv) about 2.5 mol % of a PEG lipid.

In certain embodiments, the lipid composition (e.g., LNP) comprises: (i) about 45-50 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 37.5-40.5 mol % of a helper lipid (e.g., cholesterol); and (iv) about 3-4 mol % of a PEG lipid. In certain embodiments, the lipid composition comprises: (i) about 47.5 mol % of a lipid compound of Formula (I) or Formula (II) or a pharmaceutically acceptable salt thereof; (ii) about 10 mol % of a phospholipid (e.g., distearoylphosphatidylcholine (DSPC); (iii) about 39 mol % of a helper lipid (e.g., cholesterol); and (iv) about 3.5 mol % of a PEG lipid.

In certain embodiments, lipid compositions of the present disclosure (e.g., LNP) further comprise a targeting component. In certain embodiments, the targeting component is a passive targeting component. In certain embodiments, the passive targeting component comprises a targeting lipid. In certain embodiments, the targeting lipid comprises at least one cationic targeting lipid. Examples of cationic targeting lipids for use in a lipid composition (e.g., LNP) of the present disclosure include, but are not limited to, 1,2-dioleoyl-3-trimethylammonium propane (DOTAP) and N-[1-(2,3-dioleyloxy)propyl]-N,N,N-trimethylammonium chloride (DOTMA). In certain embodiments, the targeting lipid comprises at least one anionic targeting lipid. Examples of anionic targeting lipids for use in a lipid composition (e.g., LNP) of the present disclosure include, but are not limited to, phosphatidic acid (PA), Bis(monoacylglycero)phosphate (BMP), hemi-bis(monoacylglycero)phosphates (hemi-BMP), and bis(diacylglycero)phosphates (BDP).

In certain embodiments, the targeting component of a lipid compositions (e.g., a lipid nanoparticle (LNP)) disclosed herein is an active targeting component. In certain embodiments, the active targeting component comprises a protein. In certain embodiments, the active targeting component comprises a peptide. In certain embodiments, the active targeting component comprises a small molecule. In certain embodiments, the active targeting component comprises an antibody. In certain embodiments, the active targeting component comprises an antigen-binding fragment of an antibody.

### Active Agents

In certain embodiments, the lipid compositions (e.g., a lipid nanoparticle (LNP)) disclosed herein comprise at least one active agent (e.g., RNA, DNA). In certain embodiments, the present disclosure provides lipid compositions (e.g., a lipid nanoparticle (LNP)) comprising at least one lipid compound of Formula (I) or Formula (II), or a pharmaceutically acceptable salt thereof, and at least one active agent (e.g., RNA, DNA) within the lipid composition, e.g., within the LNP.

In certain embodiments, the active agent comprises one or more polynucleotides. In certain embodiments, the active agent comprises one or more RNA. In certain embodiments, the active agent comprises one or more DNA. In certain embodiments, the active agent comprises one or more RNA and one or more DNA.

In certain embodiments, the active agent comprises mRNA. In certain embodiments, the active agent comprises an mRNA encoding an RNA-guided DNA-binding agent (e.g., a Cas nuclease, such as Cas9).

In certain embodiments, the active agent comprises gRNA. In certain embodiments, the active agent comprises dgRNA or sgRNA.

In certain embodiments, the active agent comprises an inhibitory polynucleotide, e.g., siRNA (i.e., a non-coding, short interfering RNA molecule).

### Compositions and Methods

In certain embodiments, the present disclosure provides a pharmaceutical composition comprising a lipid composition (e.g., a lipid nanoparticle (LNP)) of the present disclosure. In certain embodiments, the pharmaceutical composition comprises a lipid composition (e.g., a lipid nanoparticle (LNP)) of the present disclosure, and at least one pharmaceutically acceptable excipient. In certain embodiments, the pharmaceutical composition is an aqueous solution or suspension. In certain embodiments, the pharmaceutical composition is an oil-based solution or suspension.

In certain embodiments, the present disclosure provides a method of delivering an active agent (e.g., polynucleotide) to a target cell or tissue (e.g., extrahepatic cell or tissue). In certain embodiments, the method comprises administering to a subject an effective amount of a lipid composition (e.g., LNP) of the present disclosure. In certain embodiments, the method comprises administering to a subject an effective amount of a pharmaceutical composition comprising a lipid composition (e.g., LNP) of the present disclosure.

In certain embodiments, the present disclosure describes a composition of the present disclosure for use in delivering an active agent (e.g., polynucleotide) to a target cell or tissue (e.g., extrahepatic cell or tissue). In certain embodiments, the present disclosure describes the use a composition of the present disclosure in delivering an active agent (e.g., polynucleotide) to a target cell or tissue (e.g., extrahepatic cell or tissue). In certain embodiments, the present disclosure describes the use a composition of the present disclosure in the manufacture of a medicament for use in delivering an active agent (e.g., polynucleotide) to a target cell or tissue (e.g., extrahepatic cell or tissue). In certain embodiments, the use comprises administering to a subject an effective amount of a lipid composition (e.g., LNP) of the present disclosure. In certain embodiments, the use comprises administering to a subject an effective amount of a pharmaceutical composition comprising a lipid composition (e.g., LNP) of the present disclosure.

In certain embodiments, the target cell or tissue is an extrahepatic cell or tissue. In certain embodiments, the extrahepatic cell or tissue comprises one or more of (e.g., all of): brain cell or tissue; lung cell or tissue; bone marrow cell or tissue; spleen cell or tissue; lymph node cell or tissue; ovarian/testicular cell or tissue; muscle cell or tissue; kidney cell or tissue; pancreatic cell or tissue; and/or heart cell or tissue. In certain embodiments, the extrahepatic cell or tissue comprises one or more of (e.g., all of): brain cell or tissue; lung cell or tissue; and/or bone marrow cell or tissue. In certain embodiments, the bone marrow cell or tissue is in a femur. In certain embodiments, the extrahepatic cell or tissue comprises a brain cell or tissue. In certain embodiments, the extrahepatic cell or tissue comprises a lung cell or tissue. In certain embodiments, the extrahepatic cell or tissue comprises a bone marrow cell or tissue. In certain embodiments, the extrahepatic cell or tissue comprises spleen cell or tissue. In certain embodiments, the extrahepatic cell or tissue comprises lymph node cell or tissue. In certain embodiments, the extrahepatic cell or tissue comprises ovarian or testicular cell or tissue. In certain embodiments, the extrahepatic cell or tissue comprises muscle cell or tissue. In certain embodiments, the extrahepatic cell or tissue comprises kidney cell or tissue. In certain embodiments, the extrahepatic cell or tissue comprises a pancreatic cell or tissue. In certain embodiments, the extrahepatic cell or tissue comprises heart cell or tissue.

In certain embodiments, the present disclosure provides a method of treating a disease in a subject, the method comprising administering to the subject a therapeutically effective amount of a pharmaceutical composition comprising a lipid composition (e.g., LNP) of the present disclosure.

In certain embodiments, the present disclosure describes a composition of the present disclosure for use in the treatment of a disease in a subject by administering to the subject a therapeutically effective amount of the composition, wherein the composition comprises a lipid composition (e.g., LNP) of the present disclosure.

In certain embodiments, the present disclosure describes the use a composition of the present disclosure in the treatment of a disease in a subject by administering to the subject a therapeutically effective amount of the composition, wherein the composition comprises a lipid composition (e.g., LNP) of the present disclosure.

In certain embodiments, the present disclosure describes the use a composition of the present disclosure in the manufacture of a medicament for use in the treatment of a disease in a subject by administering to the subject a therapeutically effective amount of the medicament, wherein the medicament comprises a lipid composition (e.g., LNP) of the present disclosure.

In certain embodiments, the lipid composition (e.g., LNP) comprises a lipid compound of Formula (I) or Formula (II), or a pharmaceutically acceptable salt thereof, and an active agent (e.g., DNA, RNA).

In certain embodiments, the lipid composition (e.g., LNP) comprises a lipid compound selected from any one of Compounds 1-209, or a pharmaceutically acceptable salt thereof, and encapsulates an active agent (e.g., DNA, RNA).

In certain embodiments, the lipid composition (e.g., LNP) comprises a lipid compound selected from any one of Compounds 7, 8, 10, 13, 14, 26, 33, 38, 39, 40, 48, 60, 61, 89, 103, or 109, or a pharmaceutically acceptable salt thereof, and encapsulates an active agent (e.g., DNA, RNA).

In certain embodiments, the present disclosure provides a method of producing a therapeutic composition, the method comprising encapsulating an active agent (e.g., DNA, RNA) within a lipid nanoparticle (LNP) of the present disclosure. In certain embodiments, the present disclosure provides a method of producing a prophylactic composition, the method comprising encapsulating an active agent (e.g., DNA, RNA) within a lipid nanoparticle (LNP) of the present disclosure. In certain embodiments, the present disclosure provides a method of producing a vaccine, the method comprising encapsulating an active agent (e.g., DNA, RNA) within a lipid nanoparticle (LNP) of the present disclosure.

In certain embodiments, the lipid composition (e.g., LNP) of the therapeutic composition, prophylactic composition, or vaccine comprises a lipid compound of Formula (I) or Formula (II), or a pharmaceutically acceptable salt thereof. In certain embodiments, the active agent (e.g., DNA, RNA) is encapsulated into the lipid nanoparticle (LNP) by mixing a solution comprising the active agent (e.g., DNA, RNA) with a solution/suspension comprising the lipid nanoparticles, or comprising precursor elements of the lipid nanoparticles. Examples of solutions or solvents that can be used in forming LNPs or in encapsulating active agents into LNPs include, but are not limited to: water, PBS, Tris buffer, NaCl, citrate buffer, acetate buffer, ethanol, chloroform, diethylether, cyclohexane, tetrahydrofuran, methanol, and isopropanol.

In certain embodiments, the active agent comprises DNA. In certain embodiments, the active agent comprises RNA. In certain embodiments, the active agent comprises DNA and RNA. In certain embodiments, the RNA comprises mRNA. In certain embodiments, the RNA comprises inhibitory RNA, e.g., siRNA.

### III. Definitions

Unless stated otherwise, the following terms and phrases have the meanings described below. The definitions are not meant to be limiting in nature and serve to provide a clearer understanding of certain aspects of the present disclosure.

Administering: As used herein, the term “administering” refers to providing a composition to a subject.

Alkenoxy: As used herein, the term “alkenoxy” refers to an alkenyl moiety attached through a divalent oxygen bridge (e.g., —O—C₁₋₂₀ alkenyl). Examples of such groups include, but are not limited to, ethenoxy, propenoxy, and the like.

Alkenyl: As used herein, the term “alkenyl” refers to an unsaturated hydrocarbon chain (branched or unbranched) having one or more carbon-carbon double bonds within the chain. In certain embodiments, an alkenyl group may be optionally substituted with one or more substituents. Examples of alkenyl groups include, but are not limited to, ethylenyl, propenyl, butenyl, pentenyl, hexenyl, and the like.

Alkenylene: As used herein, the term “alkenylene” refers to a divalent alkenyl group. Examples of alkenylene groups include, but are not limited to, ethenylene, propenylene, butenylene, pentenylene, hexenylene, and the like.

Alkoxy: As used herein, the term “alkoxy” refers to an alkyl moiety attached through a divalent oxygen bridge (e.g., R—O—C₁₋₂₀). Examples of such groups include, but are not limited to, methoxy, ethoxy, propoxy, and the like.

Alkyl: As used herein, the term “alkyl” refers to a saturated hydrocarbon chain (branched or unbranched). In certain embodiments, an alkyl group may be optionally substituted with one or more substituents (i.e., the alkyl group may be unsubstituted or may be substituted with one or more substituents). In certain embodiments, an alkyl group may be optionally substituted with one or more halo groups (e.g., F). Examples of alkyl groups include, but are not limited to, methyl, ethyl, propyl (n-propyl, iso-propyl), butyl (n-butyl, sec-butyl, iso-butyl, tert-butyl), pentyl (n-pentyl, isopentyl, neopentyl), and the like.

Alkylene: As used herein, the term “alkylene” refers to divalent alkyl group. Examples of alkylene groups include, but are not limited to, methylene, ethylene, propylene (n-propylene, iso-propylene), butylene (n-butylene, sec-butylene, iso-butylene, tert-butylene), pentylene (n-pentylene, isopentylene, neopentylene), and the like.

Alkynyl: As used herein, the term “alkynyl” refers to an unsaturated hydrocarbon chain (branched or unbranched) having one or more carbon-carbon triple bonds within the chain. Examples of alkynyl groups include, but are not limited to, ethynyl, propynyl, butynyl, pentynyl, hexynyl and the like.

Alkynylene: As used herein, the term “alkynylene” refers to a divalent alkynyl group. Examples of alkynylene groups include, but are not limited to, ethynylene, propynylene, butynylene, pentynylene, hexynylene and the like.

Approximately/About: As used herein, the terms “approximately” and “about” are used interchangeably and refer to a value that is within +/−10% of the recited value as applied to one or more values of interest. In certain embodiments, the term refers to a range of values that fall within +/−10%, +/−9%, +/−8%, +/−7%, +/−6%, +/−5%, +/−4%, +/−3%, +/−2%, +/−1%, or less of the stated reference value, unless otherwise expressly stated or otherwise clearly evident from the context.

Aryl: As used herein, the term “aryl” refers to a monocyclic aromatic hydrocarbon ring or a multicyclic group that contains at least one aromatic hydrocarbon ring. In certain embodiments, an aryl group may be optionally substituted with one or more substituents. Examples of aryl groups include, but are not limited to, phenyl and naphthyl.

Arylene: The term “arylene” is a multivalent (e.g., divalent or trivalent) aryl group.

Cycloalkyl: As used herein, the term “cycloalkyl” refers to a saturated monocyclic or multicyclic (e.g., bicyclic or tricyclic) hydrocarbon ring. In certain embodiments, a cycloalkyl group may be optionally substituted with one or more substituents. In certain embodiments, a cycloalkyl group may be optionally substituted with one or more C₁-C₁₂ alkyl groups (e.g., methyl, ethyl, n-pentyl, n-butyl, n-pentyl, etc.). The number of ring atoms in the cycloalkyl ring can be specified using “Cr-Cy cycloalkyl” nomenclature where x and y are integers specifying the number of ring atoms. Examples of cycloalkyl groups include, but are not limited to, cyclopropyl, cyclobutyl, cyclopentyl, cyclohexyl, adamantyl and the like. In certain embodiments, a cycloalkyl group comprises a fused cycloalkyl group (e.g., fused bicyclic or fused tricyclic group). In certain embodiments, a cycloalkyl group comprises a bridged cycloalkyl group (e.g., bridged bicyclic group). In certain embodiments, a cycloalkyl group comprises a spiro cycloalkyl group (e.g., spiro bicyclic group).

Cycloalkylene: As used herein, the term “cycloalkylene” refers to a divalent cycloalkyl group. Examples of such groups include, but are not limited to, cyclopropylene, cyclobutylene, cyclopentylene, cyclohexylene, adamantylene, and the like.

Dienyl: As used herein, the term “dienyl” refers to an unsaturated hydrocarbon chain (branched or unbranched) having one or more carbon-carbon double bonds within the chain. Examples of dienyl groups include, but are not limited to, 1,3-pentadienyl, 1,4-hexadienyl, (2Z,5Z)-undeca-2,5-dienyl, and the like.

Effective amount: As used herein, the term “effective amount” or “therapeutically effective amount” of an agent is an amount sufficient to effect a beneficial or desired result (e.g., biological, medical, or clinical result). As such, an effective amount depends upon the context in which it is being applied (e.g., route of administration, seriousness of the condition, biochemistry, and medical history of subject, etc.), and can be determined by standard clinical techniques by those with skill in the art (e.g., extrapolated from dose-response curves derived from testing).

Halo: As used herein, the term “halo” refers to fluoro, chloro, bromo, and/or iodo.

Heterocyclic Heterocycle Heterocyclyl: As used herein, the term “heterocyclic,” “heterocycle,” or “heterocyclyl” refers to a saturated or unsaturated non-aromatic ring (monocyclic or bicyclic) containing one or more (e.g., from 1 to 4) heteroatoms (e.g., N, O, or S). The number of ring atoms in the heterocyclic ring can be specified using “x-y membered” nomenclature where x and y are integers specifying the number of ring atoms. For example, a 3-6 membered heterocycle group refers to a saturated or unsaturated 3- to 6-membered ring structure containing one or more heteroatoms, such as nitrogen, oxygen, and sulfur. Examples of heterocyclic groups include, but are not limited to, pyrrolinyl, pyrrolidinyl, pyrazolidinyl, oxazolyl, thiazolyl, piperidinyl, piperazinyl, morpholinyl, tetrahydropyranyl, and the like.

Heterocyclylene: As used herein, the term “heterocyclylene” refers to a divalent heterocycle group. Examples of such groups include, but are not limited to, pyrrolinylene, pyrrolidinylene, pyrazolidinylene, oxazolylene, thiazolylene, piperidinylene, piperazinylene, morpholinylene, and the like.

Hydroxyalkyl: As used herein, the term “hydroxyalkyl” refers to an R—C₁₋₂₀—OH group. Examples of such groups include, but are not limited to, hydroxymethyl, 2-hydroxyethyl, 3-hydroxypropyl, and the like.

Lipid nanoparticle: As used herein, the terms “lipid nanoparticle” or “LNP” refer to particles comprising a plurality of lipid molecules physically associated with each other by intermolecular forces. In certain embodiments, lipid nanoparticles are microspherical vesicles that comprise liposomal lamellar phase lipid bilayers (unilamellar or multilamellar). In other embodiments, lipid nanoparticles are nanospherical vesicles that comprise liposomal lamellar phase lipid bilayers (unilamellar or multilamellar).

mol %: As used herein, the terms “mole percentage” or “mol %” refer to the mole fraction of a specific element within a mixture, stated as a percentage of the total number of moles in the mixture. The mole fraction is the number of moles of one ingredient in the given mixture relative to the total number of moles in the mixture.

Pharmaceutically acceptable: As used herein, the terms “pharmaceutically acceptable” or “therapeutically acceptable” are used to describe compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: As used herein, the term “pharmaceutically acceptable excipient” refers to an ingredient in a composition capable of suspending, carrying, diluting, stabilizing, controlling, encapsulating, or otherwise supplementing a compound or composition of the present disclosure (e.g., LNP) in a pharmaceutical composition. Pharmaceutically acceptable excipients are substantially nontoxic, non-inflammatory, and otherwise pharmaceutically acceptable (as defined above) in a subject.

Pharmaceutically acceptable salt: As used herein, the phrase “pharmaceutically acceptable salt” refers to salts which are, within the scope of sound medical judgement, suitable for use in contact with the tissues of humans and lower animals without undue toxicity, irritation, allergic response, and the like and are commensurate with a reasonable benefit/risk ratio. Compounds as described herein may contain either a basic or an acidic functionality, or both, and can be converted to a pharmaceutically acceptable salt, when desired, by using a suitable acid or base. The salts may be prepared in situ during the final isolation and purification of the compounds.

Phospholipid: As used herein, the term “phospholipid” refers to a lipid that includes a phosphate moiety and one or more carbon chains, such as unsaturated fatty acid chains. A phospholipid may include one or more double or triple bonds in the carbon chains (e.g., one or more unsaturations).

Preventing or prophylaxis: As used herein, the term “preventing,” “prevention,” or “prophylaxis” refers to partially or completely delaying onset of a disease or condition; partially or completely delaying onset of one or more symptoms, features, or clinical manifestations of a disease or condition; partially or completely delaying progression of a disease or condition; and/or decreasing the risk of developing pathology associated with a disease or condition. In certain embodiments, “preventing,” “prevention,” or “prophylaxis” of a disease or condition may be considered a subset within the meaning of the term “treatment” or “treating” of the disease or condition.

Subject: As used herein, the term “subject” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject may seek or need treatment, require treatment, is receiving treatment, will receive treatment, or is under care by a trained professional for a particular disease or condition.

The Same Substituents: As used herein, when two substituents are “the same,” the substituents have the same molecular formula and sequence of bonded atoms, but may or may not differ in the three-dimensional orientations of their atoms in space. In certain embodiments, when each of the same substituents contains one or more double bonds, the configuration of each of the one or more double bonds in one substituent is the same as the configuration of the corresponding one or more double bonds in the other substituent.

Treating: As used herein, the term “treating” or “treatment” refers to partially or completely alleviating, ameliorating, improving, relieving, delaying onset of, inhibiting progression of, reducing severity of, reducing incidence of, and/or preventing one or more symptoms or features of a particular disease or condition. Treatment may be administered to a subject who does not exhibit signs of a disease or condition and/or to a subject who exhibits only early signs of a disease or condition for the purpose of decreasing the risk of developing pathology (or further pathology) associated with the disease or condition.

### General Considerations

At various places in the present disclosure, substituents, or properties of compounds of the present disclosure are disclosed in groups or in ranges. It is intended that the present disclosure comprise each and every individual or sub-combination of the members of such groups and ranges, and that such groups or ranges include the endpoints. By way of nonlimiting example, if a group or range is from about 1 to about 10, then the group or range includes both the value of about 1 and the value of about 10.

Articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure can include embodiments in which exactly one member of the group is present in, employed in, or otherwise relevant to a given product or process. The present disclosure can include embodiments in which more than one, or the entire group members are present in, employed in, or otherwise relevant to a given product or process.

The term “comprising” is intended to be open and permits but does not require the inclusion of additional elements or steps. When the term “comprising” is used herein, the terms “consisting of” and “consisting essentially of” are also encompassed and disclosed.

The abbreviation, “e.g.,” is derived from the Latin exempli gratia, and is used herein to indicate a non-limiting example. Thus, the abbreviation “e.g.,” is synonymous with the term “for example.” The abbreviation, “i.e.,” is derived from the Latin id est, and is used herein to indicate a non-limiting rewording or clarification. Thus, the abbreviation “i.e.,” is synonymous with the term “that is.”

Where a variable is provided in the context of an organic chemical structure as having a range of numbers, that variable is understood to be an integer value in that range, inclusive of the end points. For example, “n is 0-3” means that n is 0, 1, 2, or 3.

Any embodiment of the present disclosure that falls within the prior art may be explicitly excluded from any one or more of the claims. Any embodiment of the agents, methods, and/or compositions of the present disclosure can be excluded from any one or more claims, for any reason, whether or not related to the existence of prior art.

The present specification will control in instances where publications, patent applications, patents, and other references mentioned herein are incorporated by reference and are in conflict with the present specification.

Section headings, materials, methods, and examples are illustrative only and not intended to be limiting.

### EXAMPLES

### Example 1—Preparation and Analysis of Lipid Nanoparticles (LNPs)

a. LNP Formulations

Formulations comprising lipid compounds of the present disclosure were prepared. Four general formulations were used, according to Table 2:

TABLE 2
General Formulations

| Formulation | Lipid Compound | DSPC | Cholesterol | PEG Lipid |
|---|---|---|---|---|
| Number | (mol %) | (mol %) | (mol %) | (mol %) |
| F1 | 47.5 | 10 | 40 | 2.5 |
| F2 | 50 | 10 | 38.5 | 1.5 |
| F3 | 60 | 10 | 27.5 | 2.5 |
| F4 | 47.5 | 10 | 39 | 3.5 |

LNP Formulations were produced using lipid compounds from Table 1: Compounds 1-12, Compound 14, Compound 16, Compound 18, and Compound 20. Formulation results are shown in Table 3 (PDI=polydispersity index, N/P=molar ratio of ionizable nitrogen to phosphate groups, EE %=encapsulation efficiency (measured using a fluorescence plate-based assay)):

TABLE 3
LNP Formulations

| Lipid | Formulation | Size (nm) | | PDI | | EE (%) | |
|---|---|---|---|---|---|---|---|
| Compound | Number | N/P:6 | N/P:3 | N/P:6 | N/P:3 | N/P:6 | N/P:3 |
| Compound 1 | F1 | 74 ± 1 | 85 ± 4 | 0.072 | 0.084 | 100 | 97 |
| | F2 | 89 ± 2 | 99 ± 2 | 0.069 | 0.052 | 100 | 98 |
| | F3 | 107 ± 3 | 136 ± 3 | 0.051 | 0.037 | 100 | 97 |
| | F4 | 58 ± 2 | 78 ± 1 | 0.151 | 0.153 | 100 | 98 |
| Compound 2 | F1 | 75 ± 8 | — | 0.16 | — | 98 | — |
| | F2 | 84 ± 1 | — | 0.04 | — | 98 | — |
| | F3 | 91 ± 0 | — | 0.04 | — | 98 | — |
| | F4 | 65 ± 2 | — | 0.18 | — | 97 | — |
| Compound 3 | F1 | 74 ± 1 | — | 0.09 | — | 98 | — |
| | F2 | 92 ± 1 | — | 0.01 | — | 99 | — |
| | F3 | 102 ± 1 | — | 0.07 | — | 98 | — |
| | F4 | 73 ± 1 | — | 0.14 | — | 98 | — |
| Compound 4 | F1 | 78 ± 2 | 86 ± 10 | 0.063 | 0.092 | 100 | 98 |
| | F2 | 93 ± 1 | 97 ± 1 | 0.069 | 0.041 | 100 | 98 |
| | F3 | 108 ± 2 | 116 ± 6 | 0.08 | 0.089 | 100 | 94 |
| | F4 | 73 ± 2 | 73 ± 0 | 0.12 | 0.068 | 100 | 98 |
| Compound 5 | F1 | 70 ± 0 | — | 0.12 | — | 98 | — |
| | F2 | 88 ± 3 | — | 0.08 | — | 96 | — |
| | F3 | 97 ± 3 | — | 0.06 | — | 98 | — |
| | F4 | 66 ± 3 | — | 0.16 | — | 99 | — |
| Compound 6 | F1 | 69 ± 3 | 77 ± 2 | 0.172 | 0.09 | 100 | 99 |
| | F2 | 83 ± 1 | 89 ± 2 | 0.012 | 0.027 | 100 | 99 |
| | F3 | 85 ± 1 | 105 ± 5 | 0.078 | 0.059 | 100 | 95 |
| | F4 | 63 ± 1 | 65 ± 1 | 0.081 | 0.084 | 100 | 98 |
| Compound 7 | F1 | 75 ± 1 | — | 0.08 | — | 99 | — |
| | F2 | 97 ± 5 | — | 0.11 | — | 97 | — |
| | F3 | 99 ± 2 | — | 0.09 | — | 98 | — |
| | F4 | 75 ± 3 | — | 0.21 | — | 98 | — |
| Compound 8 | F1 | 87 ± 6 | — | 0.08 | — | 100 | — |
| | F2 | 117 ± 4 | — | 0.06 | — | 99 | — |
| | F3 | 100 ± 2 | — | 0.12 | — | 95 | — |
| | F4 | 79 ± 1 | — | 0.19 | — | 99 | — |
| Compound 9 | F1 | 68 ± 2 | — | 0.07 | — | 100 | — |
| | F2 | 76 ± 1 | — | 0.03 | — | 100 | — |
| | F3 | 92 ± 2 | — | 0.03 | — | 99 | — |
| | F4 | 63 ± 1 | — | 0.13 | — | 99 | — |
| Compound 10 | F1 | 78 ± 3 | 86 ± 4 | 0.09 | 0.208 | 100 | 98 |
| | F2 | 96 ± 0 | 102 ± 3 | 0.122 | 0.097 | 100 | 98 |
| | F3 | 100 ± 3 | 106 ± 2 | 0.092 | 0.081 | 100 | 96 |
| | F4 | 78 ± 1 | 92 ± 4 | 0.227 | 0.278 | 100 | 98 |
| Compound 11 | F1 | 77 ± 7 | — | 0.16 | — | 100 | — |
| | F2 | 88 ± 3 | — | 0.11 | — | 100 | — |
| | F3 | 91 ± 1 | — | 0.14 | — | 97 | — |
| | F4 | 74 ± 2 | — | 0.30 | — | 99 | — |
| Compound 12 | F1 | 81 ± 9 | — | 0.16 | — | 98 | — |
| | F2 | 84 ± 5 | — | 0.07 | — | 98 | — |
| | F3 | 93 ± 1 | — | 0.04 | — | 97 | — |
| | F4 | 63 ± 0 | — | 0.09 | — | 98 | — |
| Compound 14 | F1 | 78 ± 2 | — | 0.06 | — | 99 | — |
| | F2 | 90 ± 5 | — | 0.07 | — | 99 | — |
| | F3 | 83 ± 1 | — | 0.14 | — | 99 | — |
| | F4 | 68 ± 3 | — | 0.09 | — | 98 | — |
| Compound 16 | F1 | 73 ± 7 | — | 0.14 | — | 99 | — |
| | F2 | 82 ± 2 | — | 0.07 | — | 99 | — |
| | F3 | 92 ± 0 | — | 0.04 | — | 98 | — |
| | F4 | 64 ± 0 | — | 0.15 | — | 99 | — |
| Compound 18 | F1 | 67 ± 1 | — | 0.04 | — | 99 | — |
| | F2 | 78 ± 1 | — | 0.02 | — | 99 | — |
| | F3 | 96 ± 2 | — | 0.03 | — | 99 | — |
| | F4 | 65 ± 2 | — | 0.05 | — | 99 | — |
| Compound 20 | F1 | 64 ± 1 | — | 0.06 | — | 100 | — |
| | F2 | 76 ± 1 | — | 0.03 | — | 100 | — |
| | F3 | 91 ± 2 | — | 0.03 | — | 100 | — |
| | F4 | 61 ± 1 | — | 0.05 | — | 100 | — |

b. LNP Preparation

Lipids and active ingredients (e.g., RNA, DNA, etc.) were assembled into LNPs using microfluidic mixing. An ethanol phase was prepared by solubilizing ionizable lipids (i.e., lipid compounds), phospholipids (e.g., DSPC), cholesterol, and PEG lipids in ethanol, at predetermined mol % ratios given in Table 2. An aqueous phase was prepared by diluting a nucleic acid cargo (e.g., firefly luciferase mRNA) in an acidified buffer (pH 4.0 citrate buffer, 50 mM). A chip with microfluidic mixing architecture (e.g., toroidal mixer) was used to mix the two phases at predetermined flow rate ratios (e.g., 9 ml/min for the aqueous phase and 3 ml/min for the ethanol phase). Resulting LNPs comprising the lipids and encapsulating the nucleic acid cargo were dialyzed against PBS pH 7.4 for 2 hours at room temperature in a 20 kDa MWCO dialysis membrane. Then, PBS was refreshed to continue dialysis overnight at 2-8 TC. Upon completion of the buffer exchange, LNPs were optionally concentrated using a ultracentrifugal unit with a 10-100 kDa MWCO membrane.

c. Plate-Based Fluorescence (RiboGreen) Assay

Nucleic acid cargos in the LNPs were quantified by RiboGreen assay, against a nucleic acid standard curve. LNPs encapsulating nucleic acid cargo were mixed with 1× Tris-EDTA (TE) and 2% triton in TE, separately, to achieve dilutions required by the assay. Then, the samples were heated at 30° C. and vortexed at 300 rpm. Samples were plated into 96-well plates and RiboGreen reagent was added into each well. Following incubation in the dark, fluorescence was measured from the wells, and plotted against the standard curve. Nucleic acid concentration obtained from the Triton-treated samples were used for total cargo concentration quantification. Only TE diluted sample fluorescence values were used to determine the non-encapsulated (free) cargo, and their ratio was used to determine the encapsulation efficiency (EE %).

d. Dynamic Light Scattering

LNPs were diluted with PBS pH 7.4 to predetermined dilution ranges based on the process step and LNP concentration. Following mixing, the particle size and PDI were measured by a dynamic light scattering instrument.

### Example 2—Stability Studies

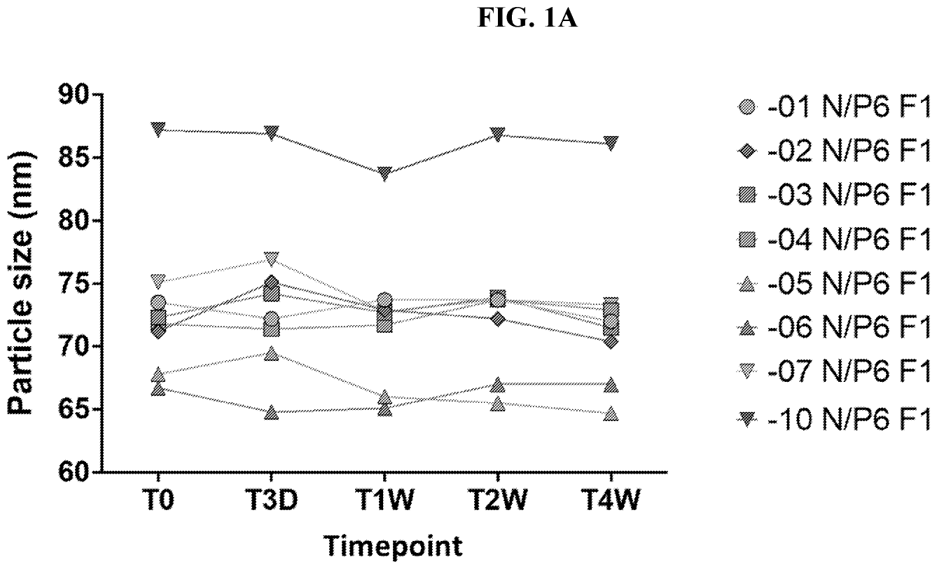

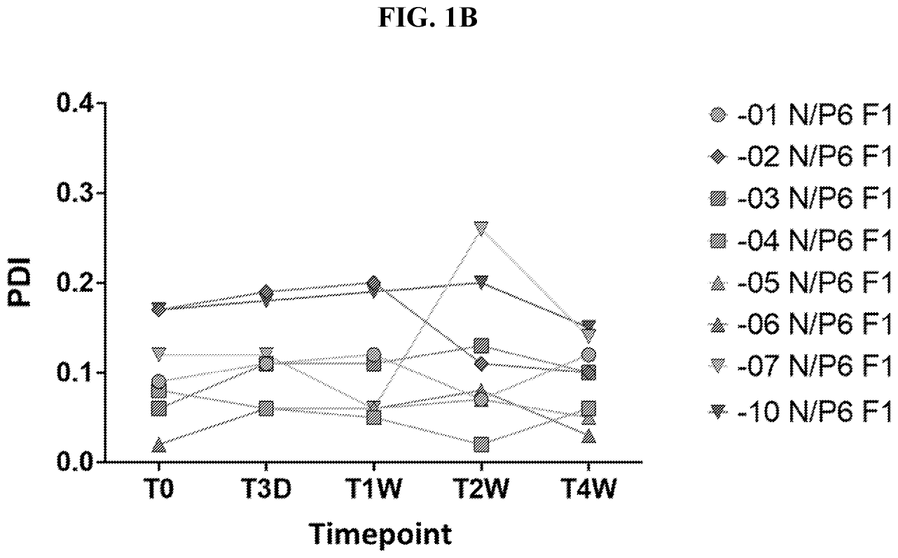

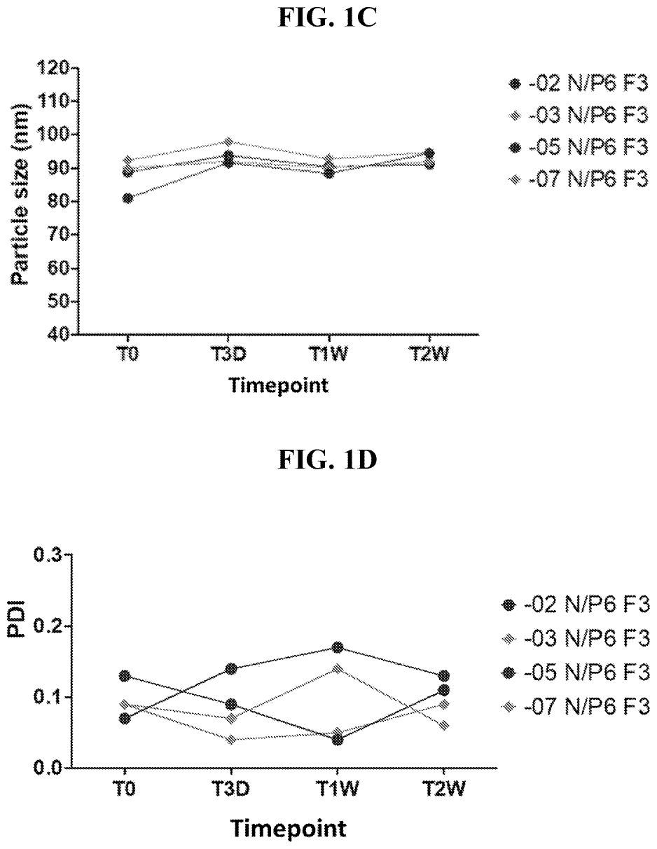

LNPs prepared from selected lipid compounds were studied for stability in 2-8° C. conditions. Compounds 1-7 and 10 (N/P: 6) were tested in Formulation F1 over 4 weeks. Particle size and PDI results are shown in FIG. 1A and FIG. 1B.

Compounds 2, 3, 5, and 7 were also tested in Formulation F3 over 2 weeks. Particle size and PDI results are shown in FIG. 1C and FIG. 1D.

### Example 3—In Vivo Activity in Mice

a. General Protocols

Small rodent biodistribution studies were performed in mice (e.g., C57BL/6J, Balb-c, CD-1, etc.). For evaluating LNP biodistribution and tissue activity upon systemic administration, C57BL/6J mice received dose administration by single intravenous injection via tail vein with LNP formulations (individual or pooled) in PBS, at different dose (0.25-3 mg/kg) levels of firefly luciferase mRNA. At predetermined time points (4-6 hours post-injection), the animals were anesthetized via isoflurane and subjected to in-life imaging sessions for bioluminescence using an In Vivo Imaging System (IVIS). All animals were dosed with D-Luciferin at 15 mg/mL via subcutaneous (SC) injection at 0.2 mL/animal. Animals had their abdomen hair shaved using an animal trimmer. They were then placed so their shaved belly faced up toward the IVIS camera. Whole body imaging sessions were performed 10-15 minutes following D-Luciferin administration.

For in detail organ distribution and activity of LNP formulations, ex vivo imaging sessions were also performed. All animals received SC D-Luciferin and were then euthanized by isoflurane overdose, followed by cardiac perfusion with saline. Following perfusion, the organs were collected and subjected to IVIS imaging for luminescence within 10-15 minutes of D-Luciferin injection.

For delivery to lungs, CD-1 mice were administered with LNP formulations via single intratracheal administration under isoflurane anesthesia. Animals were dosed with D-Luciferin at 15 mg/mL via subcutaneous (SC) injection at 0.2 mL/animal. Whole body imaging sessions were performed within 10-15 minutes following D-Luciferin injections. Then the animals were perfused with saline, and both lungs and trachea were collected and imaged for luminescence.

All IVIS images were processed with a computer software to identify the regions of interests for individual organs to detect the total flux (p/s) values as luminescence quantification. Then the total flux values were graphed to evaluate the in vivo activity in each collected organ.

b. General Activity Study

LNPs prepared from selected lipid compounds were studied for in vivo activity in mice for delivery of mRNA. LNPs were formulated using the indicated lipid compound according to each of the four formulations described in Example 2, and then the four LNP formulations were pooled together. Additional pools were created by combining sets of four LNPs corresponding to four different lipid compounds as indicated, resulting in pools of sixteen distinct compositions. Formulation properties of the resulting LNPs are shown in Table 4.

TABLE 4
LNP Compositions for in vivo mouse study

| Lipid Compound | Particle | | mRNA Content | |
|---|---|---|---|---|
| or Pool | Size (nm) | PDI | (μg/ml) | EE % |
| 1 | 89.9 | 0.143 | 102.4 | 99 |
| 2 | 82.3 | 0.11 | 86 | 96 |
| 3 | 89.1 | 0.08 | 83 | 96 |
| 4 | 93.4 | 0.022 | 99.1 | 99 |
| 5 | 84.4 | 0.12 | 80 | 96 |
| 6 | 77.9 | 0.107 | 100.1 | 99 |
| 7 | 89.3 | 0.14 | 84 | 96 |
| 8 | 101 | 0.07 | 108 | 97 |
| 9 | 79 | 0.07 | 102 | 97 |
| 10 | 91.7 | 0.176 | 101.7 | 99 |
| 11 | 86 | 0.13 | 105 | 97 |
| 12 | 83.7 | 0.119 | 91 | 97 |
| 14 | 82.9 | 0.106 | 93 | 96 |
| 16 | 82.6 | 0.125 | 84 | 96 |
| 18 | 81.1 | 0.066 | 100 | 97 |
| 20 | 79 | 0.02 | 98 | 97 |
| 1/4/6/10 Pooled | 88.2 | 0.133 | 101.7 | 99 |
| 2/3/5/7 Pooled | 84.4 | 0.06 | 91 | 96 |
| 8/9/11/20 Pooled | 87 | 0.14 | 96 | 97 |
| 12/14/16/18 Pooled | 83.9 | 0.076 | 92 | 97 |

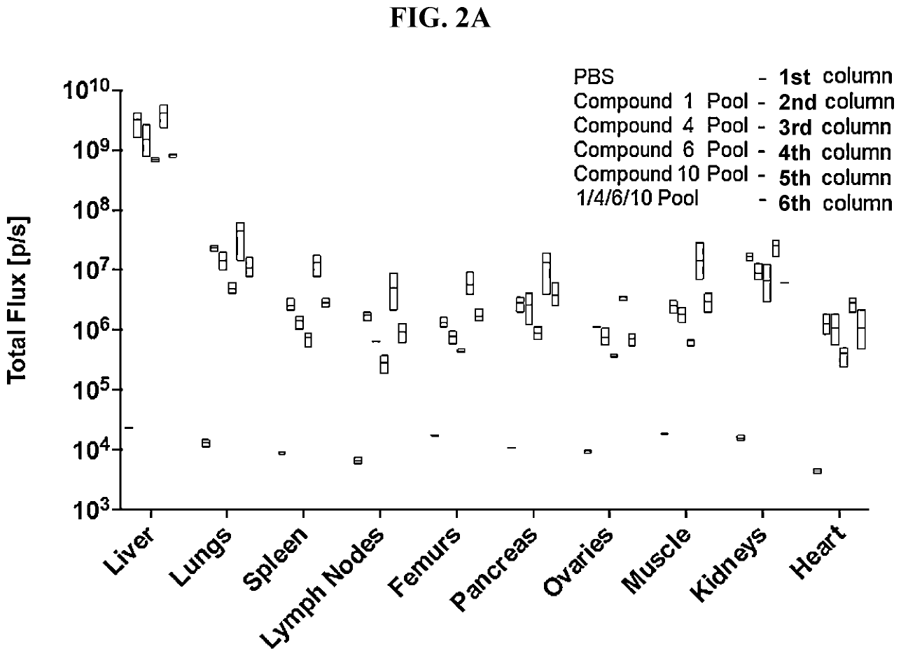

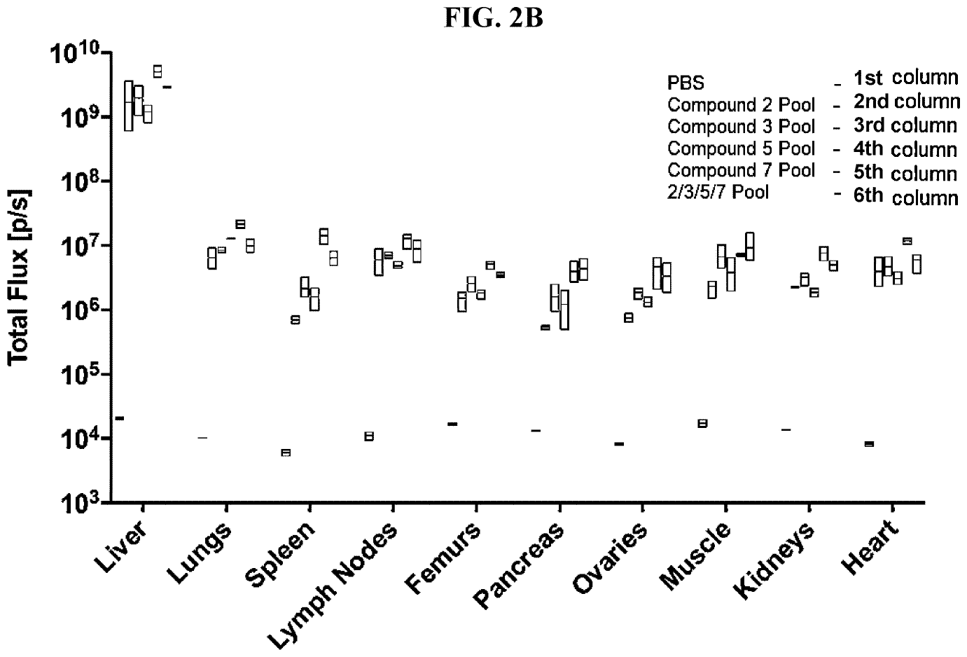

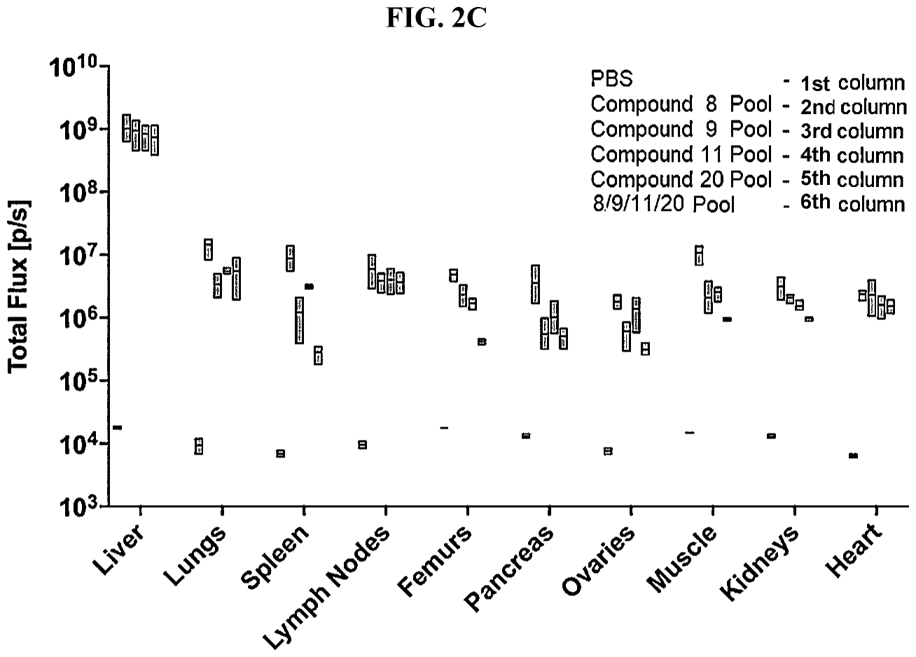

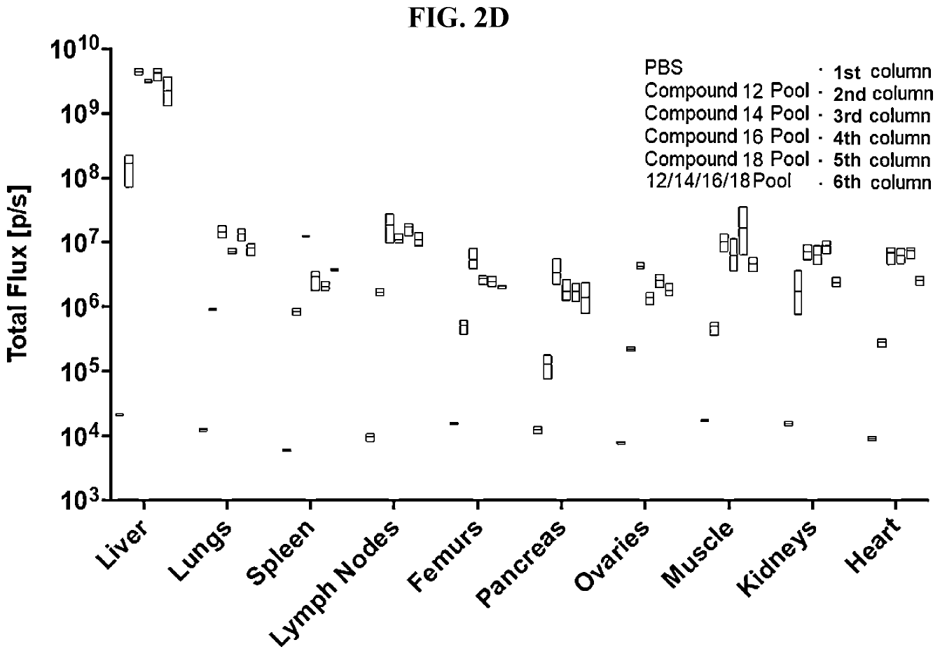

Each LNP compound pool was injected (IV) into mice (1 mg/kg), with PBS as control. Mouse organ imaging results after 6 hours showing Total Flux [p/s] are shown in FIG. 2A, FIG. 2B, FIG. 2C, and FIG. 2D.

c. Brain Activity

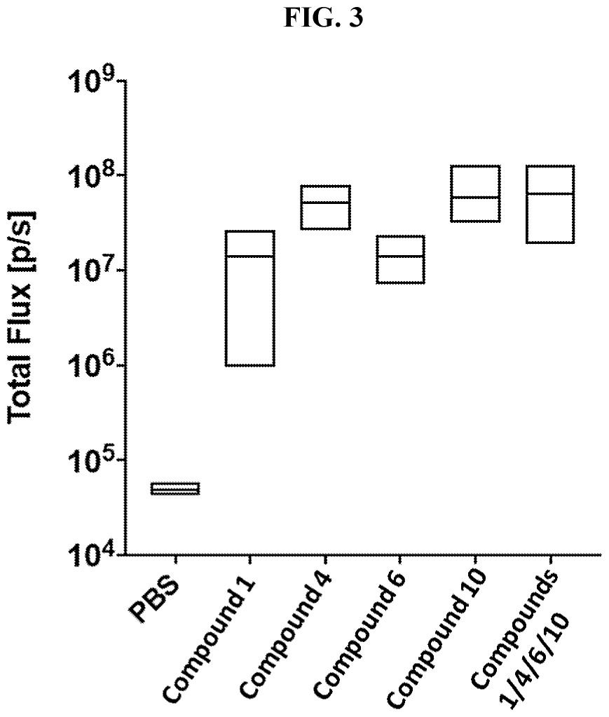

LNP compound pools for Compounds 1, 4, 6, and 10 were locally administered (ICV) into mouse brain (1 μg/animal), with PBS as control. Mouse brain imaging results showing Total Flux [p/s] are shown in FIG. 3.

d. Lung Activity

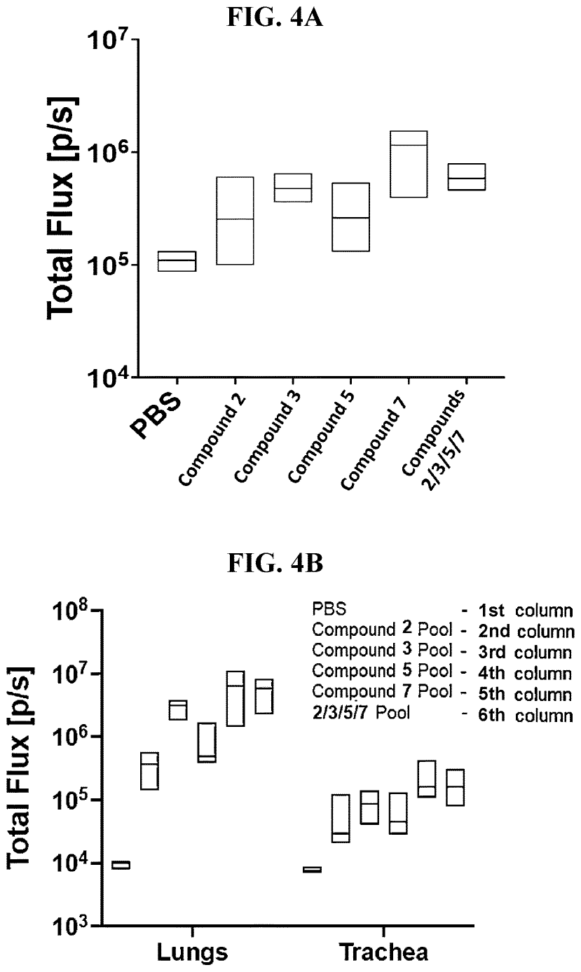

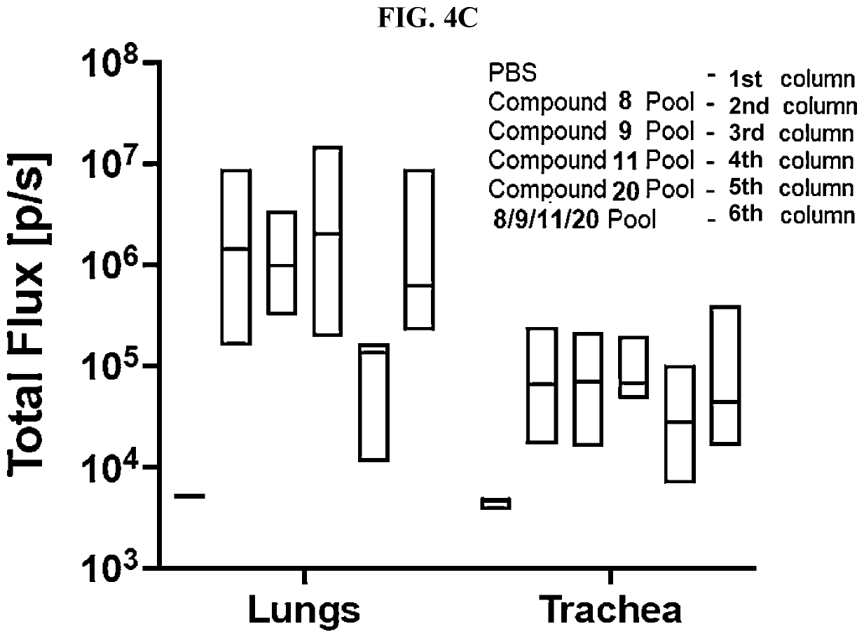

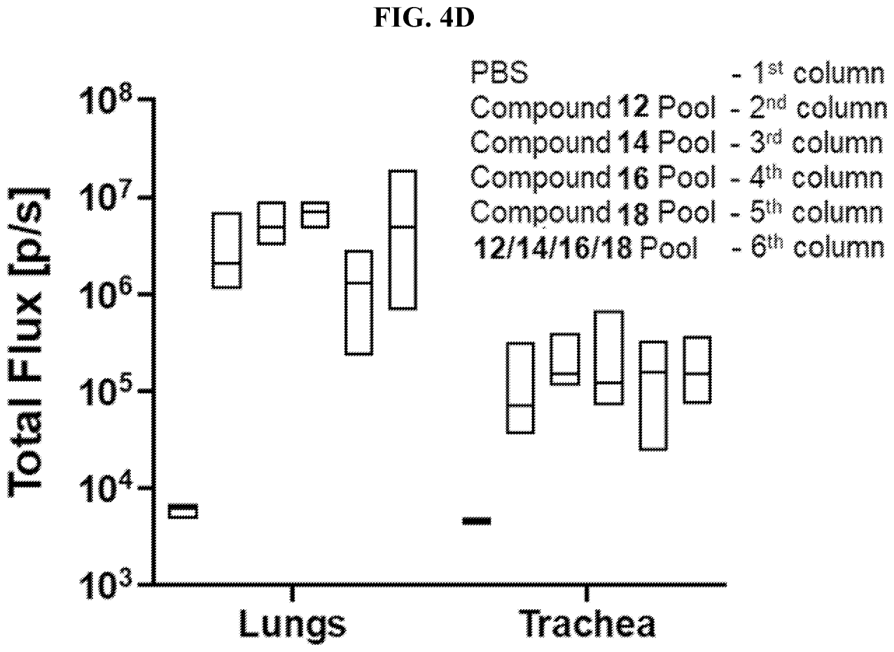

LNP compound pools for Compounds 2, 3, 5, and 7 were locally administered (intratracheally) into mouse lungs (7 μg RNA/animal), with PBS as control. Mouse lung imaging results showing Total Flux [p/s] are shown in FIG. 4A (live animal imaging) and FIG. 4B (ex vivo imaging).

LNP compound pools for Compounds 8, 9, 11, and 20 were locally administered (intratracheally) into mouse lungs (7 μg RNA/animal), with PBS as control. Mouse lung imaging results showing Total Flux [p/s] are shown in FIG. 4C.

LNP compound pools for Compounds 12, 14, 16, and 18 were locally administered (intratracheally) into mouse lungs (7 μg RNA/animal), with PBS as control. Mouse lung imaging results showing Total Flux [p/s] are shown in FIG. 4D.

### Example 4—Formulation F2 Study

a. Formulation F2 Study Design

LNPs incorporating lipid Compounds 7, 8, 10, 14, and 18 were prepared in Formulation F2 from Example 1. An LNP formulation incorporating MC3 as lipid compound (Cayman Chemical) was also prepared using Formulation F2 for comparison. PBS was used as a control. Formulation properties are shown in Table 5.

TABLE 5
LNP Compositions for Formulation F2 Study

| Lipid Compound | Particle Size (nm) | PDI | EE % |
|---|---|---|---|
| MC3 | 87.8 | 0.098 | 98 |
| 7 | 120.8 | 0.086 | 98 |
| 8 | 119 | 0.071 | 98 |
| 10 | 125.5 | 0.139 | 98 |
| 14 | 107.6 | 0.178 | 99 |
| 18 | 92.2 | 0.059 | 98 |

b. General Formulation F2 Activity

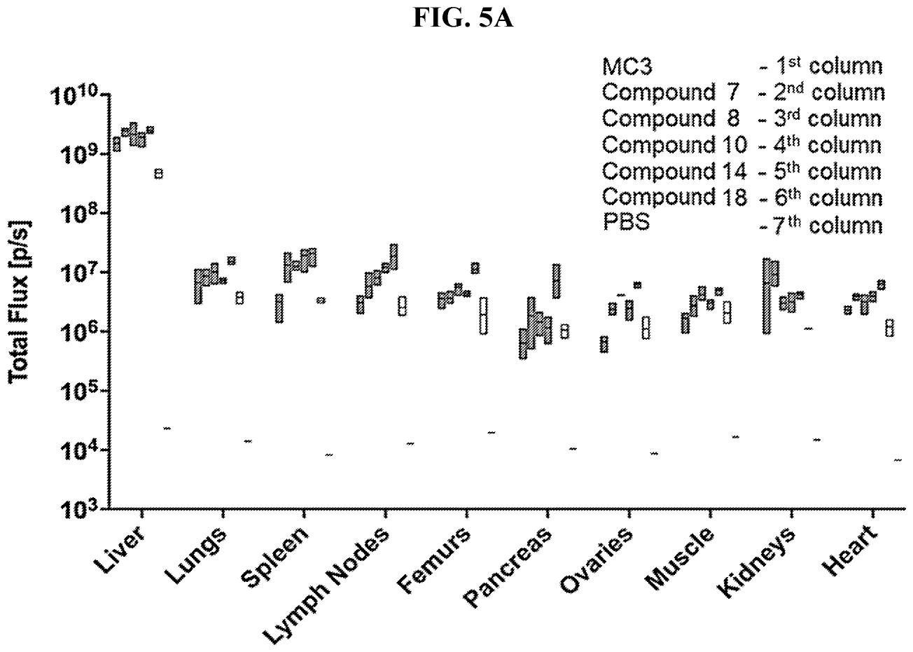

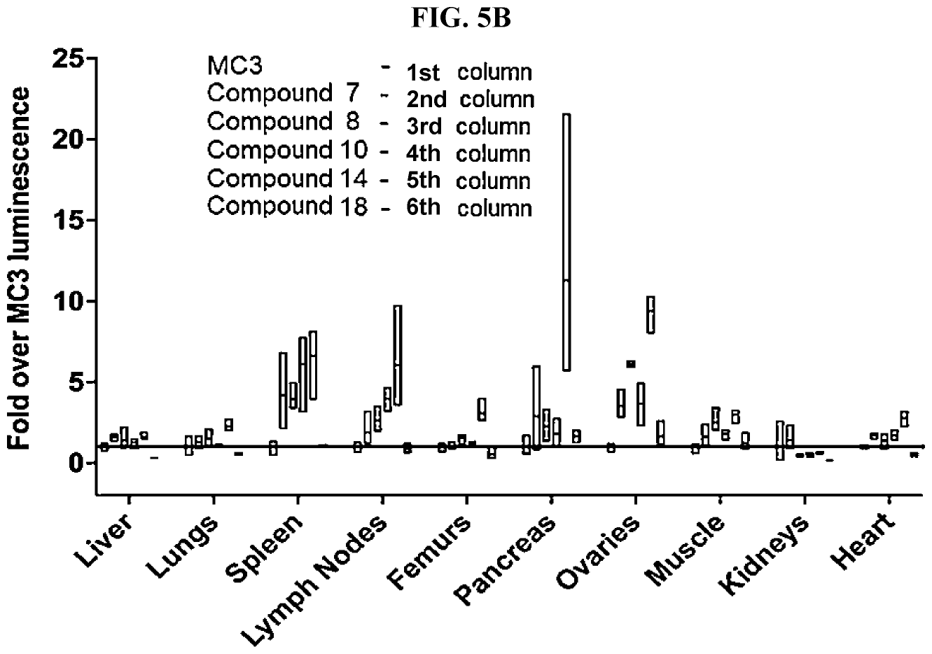

Each LNP formulation was injected (IV) into mice (0.3 mg/kg), with PBS as control. Imaging results were collected according to the protocols in Example 4. Mouse organ imaging results showing Total Flux [p/s] after 6 hours are shown in FIG. 5A. Mouse organ imaging results normalized to MC3 activity are shown in FIG. 5B.

c. Formulation F2 Lung Study

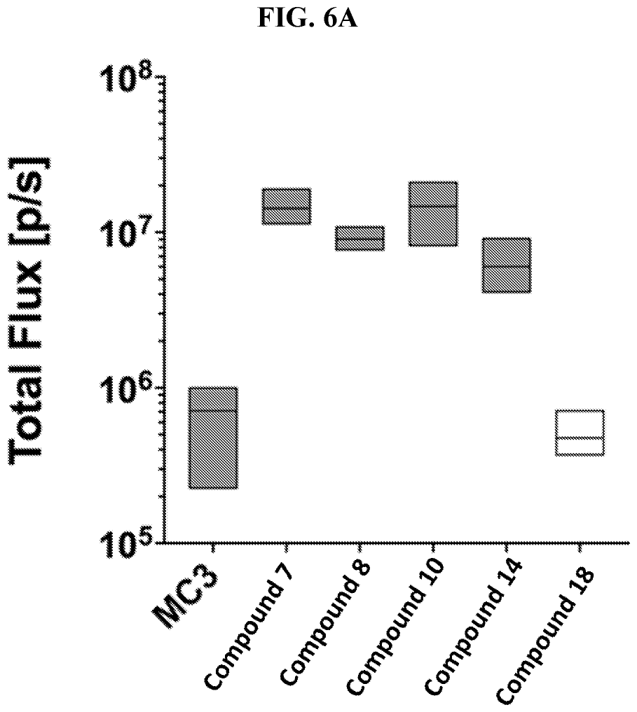

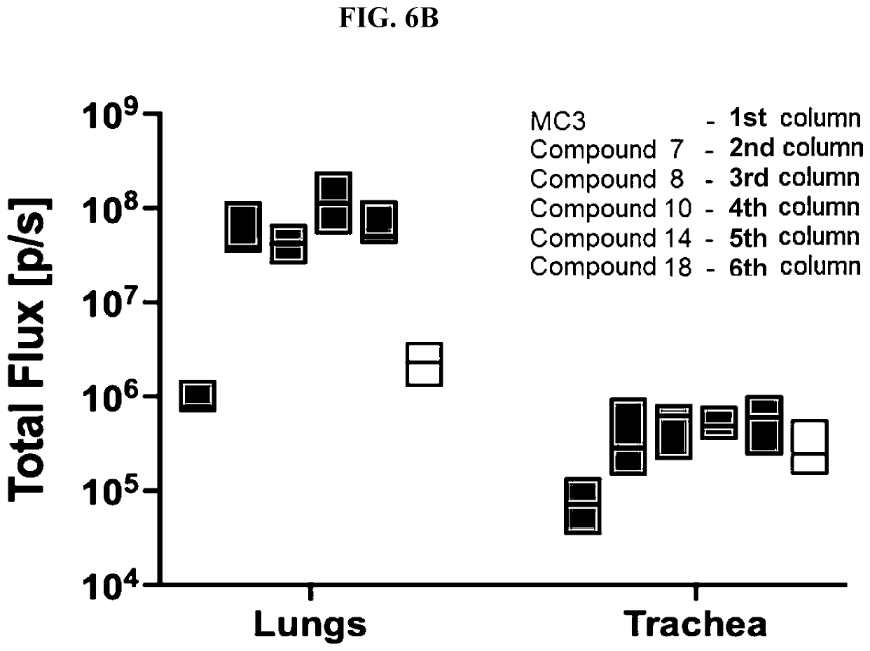

Each LNP formulation was locally administered (intratracheally) into mouse lungs (7 μg RNA/animal), MCS for comparison and with PBS as control. Imaging results were collected according to the protocols in Example 4. Mouse lung and trachea imaging results showing Total Flux [p/s] are shown in FIG. 6A (live animal imaging) and FIG. 6B (ex vivo imaging).

### Example 5—In Vivo Activity Screening in Mice

Small rodent biodistribution studies were performed in mice according to the General Study Protocols in Example 3(a). LNPs incorporating lipid compounds were prepared in Formulation F2, with MC3 as lipid compound (Cayman Chemical) prepared using Formulation F2 for comparison. LNP pools (with firefly luciferase mRNA encapsulated) were injected into mice at varying doses, and organ mean total flux (p/s) results (measuring luciferase activity) were then collected according to Example 3(a).

LNP dose, LNP pool composition, and organ mean total flux results for multiple mouse organs are shown in Table 6. LNPs comprising MC3 used as a control and for cross-mouse comparison. Dose is in mg/kg; LC is Lipid Compound according to Table 1.

TABLE 6
in vivo Activity Screening

| Mouse/ | | Organ Mean Total Flux (p/s) (×10⁶) | | | | | | | |
|---|---|---|---|---|---|---|---|---|---|
| Dose | LC | Liver | Lung | Kidney | Muscle | Spleen | Pancreas | Femur | Heart |
| 1/0.3 | MC3 | 6896 | 17.2 | 1.9 | 7.5 | 22.3 | 2.6 | 8.5 | 2.8 |
| | 14 | 3910 | 31.9 | 2.8 | 9.5 | 31.5 | 14.9 | 9.3 | 1.8 |
| | 1 | 17.5 | 0.5 | 0.1 | 0.6 | 1.7 | 0.2 | 0.8 | 0.1 |
| | 2 | 99.8 | 2.6 | 0.7 | 0.5 | 15.6 | 0.2 | 0.8 | 0.2 |
| | 3 | 29.1 | 0.7 | 0.1 | 0.5 | 1.4 | 0.2 | 1.3 | 0.1 |
| | 4 | 105.0 | 39.9 | 0.8 | 18.4 | 17.6 | 5.0 | 15.6 | 4.4 |
| | 6 | 2966 | 17.6 | 1.3 | 8.3 | 14.1 | 2.1 | 6.1 | 2.1 |
| | 7 | 140.0 | 4.9 | 0.7 | 2.5 | 29.0 | 1.8 | 2.4 | 0.2 |
| | 8 | 72.4 | 20.1 | 0.8 | 22.7 | 10.7 | 4.4 | 11.1 | 2.8 |
| | 9 | 3583 | 29.8 | 3.4 | 8.3 | 32.5 | 6.7 | 7.6 | 1.4 |
| | 10 | 12560 | 41.5 | 81.1 | 6.2 | 12.3 | 2.7 | 7.1 | 4.3 |
| | 11 | 5140 | 27.7 | 2.4 | 21.8 | 32.3 | 6.0 | 5.3 | 2.3 |
| | 12 | 61.8 | 0.7 | 0.2 | 2.7 | 0.7 | 0.2 | 1.3 | 0.1 |
| 2/1.0 | MC3 | 15500 | 60.5 | 16.6 | 16.6 | 23.0 | 7.4 | 9.9 | 17.0 |
| | 14 | 2883 | 96.9 | 7.0 | 144.9 | 107.0 | 32.1 | 62.6 | 5.9 |
| | 17 | 11856 | 59.3 | 7.6 | 30.7 | 5.5 | 3.0 | 8.0 | 13.0 |
| | 14/8 | 26 | 24.1 | 0.7 | 31.2 | 10.2 | 3.7 | 11.4 | 1.3 |
| | 60 | 1118 | 72.8 | 5.5 | 28.7 | 367.7 | 86.2 | 56.4 | 2.9 |
| | 61 | 542.7 | 123.9 | 4.8 | 28.1 | 85.1 | 28.8 | 34.2 | 1.9 |
| 3/1.0 | MC3 | 9707 | 106.4 | 7.3 | 23.6 | 22.8 | 2.7 | 6.9 | 8.5 |
| | 14 | 4307 | 49.2 | 7.7 | 193.7 | 123.8 | 23.4 | 41.8 | 8.6 |
| | 50 | 2413 | 12.1 | 2.1 | 3.8 | 9.0 | 8.8 | 3.8 | 3.1 |
| | 51 | 15467 | 59.6 | 7.2 | 16.5 | 9.7 | 4.9 | 7.5 | 16.0 |
| | 52 | 12143 | 56.0 | 8.4 | 15.1 | 6.1 | 4.7 | 6.0 | 9.0 |
| | 53 | 1286 | 12.1 | 2.2 | 5.5 | 9.3 | 2.3 | 2.5 | 2.1 |
| | 54 | 24850 | 81.5 | 60.3 | 19.2 | 9.6 | 3.6 | 9.0 | 19.3 |
| | 55 | 4527 | 19.9 | 2.4 | 17.4 | 6.2 | 2.6 | 6.1 | 4.4 |
| | 56 | 15800 | 66.6 | 9.4 | 19.7 | 13.0 | 3.4 | 6.4 | 17.7 |
| | 57 | 25033 | 71.8 | 11.4 | 16.4 | 6.5 | 2.7 | 7.4 | 16.1 |
| | 58 | 1493 | 41.0 | 2.6 | 29.1 | 10.5 | 9.2 | 11.5 | 2.8 |
| 5/0.5 | MC3 | 5667 | 34.5 | 18.6 | 10.0 | 17.6 | 1.9 | 4.8 | 5.7 |
| | 14 | 1303 | 18.9 | 1.8 | 34.1 | 20.8 | 1.9 | 11.9 | 4.4 |
| | 31 | 26.8 | 1.2 | 0.1 | 1.0 | 2.0 | 0.2 | 0.9 | 0.1 |
| | 41 | 3493 | 18.0 | 2.8 | 10.0 | 2.5 | 0.8 | 3.8 | 2.2 |
| | 42 | 1778 | 13.4 | 1.9 | 25.8 | 4.5 | 1.8 | 6.2 | 1.7 |
| | 43 | 669.3 | 12.8 | 1.6 | 8.0 | 9.5 | 2.0 | 3.1 | 0.7 |
| | 47 | 974.3 | 25.7 | 2.5 | 62.4 | 67.0 | 4.4 | 15.1 | 1.7 |
| 6/0.5 | MC3 | 4796 | 28.6 | 3.9 | 8.2 | 11.7 | 3.6 | 3.3 | 5.2 |
| | 14 | 676.3 | 17.3 | 3.2 | 27.6 | 28.7 | 14.7 | 9.1 | 1.6 |
| | 24 | 4153 | 28.8 | 5.7 | 12.1 | 10.7 | 7.9 | 6.3 | 7.1 |
| | 25 | 911.0 | 12.5 | 5.9 | 5.1 | 80.3 | 14.3 | 4.4 | 1.4 |
| | 28 | 278.3 | 10.4 | 0.9 | 11.6 | 3.5 | 0.8 | 3.5 | 0.5 |
| | 29 | 1913 | 11.4 | 3.2 | 10.3 | 3.3 | 1.4 | 4.2 | 3.9 |
| | 33 | 1024 | 16.9 | 3.0 | 53.7 | 30.1 | 9.2 | 21.3 | 2.8 |
| | 34 | 1610 | 20.2 | 6.7 | 28.3 | 39.4 | 12.8 | 10.5 | 3.3 |
| | 35 | 850.3 | 11.0 | 1.3 | 15.5 | 5.4 | 2.5 | 4.2 | 1.3 |
| | 36 | 2330 | 16.5 | 2.7 | 6.0 | 6.1 | 1.2 | 3.6 | 3.7 |
| | 38 | 6770 | 33.3 | 8.1 | 23.5 | 27.2 | 2.6 | 9.8 | 11.0 |
| | 40 | 3230 | 27.7 | 5.1 | 34.4 | 6.4 | 4.6 | 10.9 | 6.0 |
| 9/0.5 | MC3 | 3903 | 12.7 | 2.1 | 5.6 | 5.4 | 1.1 | 3.8 | 4.1 |
| | 14 | 721.0 | 10.4 | 2.5 | 31.2 | 32.2 | 10.2 | 13.3 | 1.2 |
| | 62 | 38.0 | 2.5 | 0.3 | 1.4 | 3.3 | 0.5 | 0.6 | 0.1 |
| | 63 | 351.7 | 37.3 | 2.4 | 37.4 | 40.7 | 15.8 | 10.4 | 1.9 |
| | 64 | 438.7 | 6.2 | 2.2 | 14.7 | 9.3 | 2.6 | 6.9 | 0.9 |
| | 66 | 68.9 | 14.1 | 1.8 | 11.3 | 7.8 | 4.0 | 5.5 | 1.3 |
| 11/0.5 | MC3 | 2603 | 12.1 | 1.0 | 2.7 | 4.0 | 0.7 | 2.2 | 2.9 |
| | 14 | 360.7 | 25.4 | 0.9 | 24.2 | 26.2 | 4.2 | 10.1 | 1.8 |
| | 21 | 818.7 | 6.6 | 0.7 | 4.1 | 2.0 | 2.8 | 2.0 | 0.8 |
| | 23 | 1727 | 15.7 | 1.2 | 3.0 | 1.7 | 0.6 | 1.2 | 2.1 |
| | 26 | 481.0 | 26.9 | 4.4 | 6.2 | 152.7 | 8.2 | 20.0 | 1.2 |
| | 27 | 1441 | 16.4 | 1.2 | 2.0 | 1.0 | 0.5 | 1.1 | 1.7 |
| | 37 | 1737 | 10.5 | 0.8 | 5.2 | 2.5 | 0.6 | 2.2 | 1.9 |
| | 39 | 3390 | 19.6 | 2.3 | 11.0 | 2.9 | 1.1 | 2.6 | 6.5 |
| | 44 | 415.7 | 6.5 | 1.3 | 22.6 | 36.0 | 3.1 | 5.6 | 0.7 |
| | 45 | 809.3 | 7.2 | 0.9 | 5.6 | 6.0 | 0.5 | 1.0 | 1.1 |
| | 46 | 3107 | 17.8 | 2.1 | 4.2 | 1.8 | 0.9 | 1.8 | 4.1 |
| 12/0.5 | MC3 | 2307 | 13.7 | 53.9 | 4.1 | 8.6 | 9.6 | 5.8 | 2.4 |
| | 14 | 585.3 | 32.4 | 2.2 | 35.5 | 21.1 | 3.5 | 9.3 | 1.8 |
| | 13 | 620.0 | 16.7 | 4.5 | 17.0 | 21.3 | 1.1 | 4.8 | 1.0 |
| | 67 | 220.0 | 11.8 | 1.9 | 22.8 | 50.1 | 10.4 | 10.8 | 0.4 |
| | 73 | 127.7 | 10.6 | 1.7 | 6.8 | 49.2 | 15.7 | 7.3 | 0.5 |
| | 81 | 727.0 | 12.1 | 4.0 | 5.7 | 115.9 | 12.9 | 11.1 | 1.2 |
| | 91 | 404.7 | 26.3 | 12.3 | 7.8 | 123.6 | 76.4 | 15.8 | 2.6 |
| | 93 | 528.7 | 34.3 | 3.5 | 11.0 | 136.6 | 25.8 | 27.8 | 1.3 |
| | 96 | 213.0 | 9.0 | 0.9 | 11.9 | 6.6 | 0.9 | 3.4 | 0.4 |
| | 98 | 1007 | 16.3 | 1.2 | 5.0 | 7.4 | 0.9 | 3.1 | 1.5 |
| | 99 | 177.0 | 31.4 | 1.2 | 6.5 | 47.5 | 4.3 | 7.2 | 0.4 |
| | 100 | 273.0 | 37.8 | 2.3 | 4.0 | 163.7 | 25.2 | 13.8 | 0.7 |
| | 101 | 306.7 | 26.5 | 0.9 | 5.2 | 53.2 | 14.3 | 9.7 | 0.8 |
| 13/0.5 | MC3 | 1767 | 8.3 | 0.9 | 2.8 | 5.7 | 0.8 | 2.7 | 5.0 |
| | 14 | 439.7 | 16.8 | 5.7 | 16.7 | 19.7 | 5.3 | 8.3 | 1.1 |
| | 15 | 992.3 | 3.7 | 0.4 | 1.1 | 1.6 | 0.1 | 1.3 | 1.4 |
| | 49 | 185.5 | 11.8 | 1.4 | 2.9 | 57.1 | 7.5 | 10.2 | 0.5 |
| | 50 | 569.7 | 4.7 | 0.7 | 2.3 | 4.7 | 1.5 | 1.2 | 1.0 |
| | 59 | 1967 | 9.9 | 1.5 | 4.7 | 2.8 | 0.7 | 2.6 | 2.6 |
| | 102 | 334.3 | 7.1 | 1.0 | 9.0 | 5.5 | 1.2 | 2.9 | 0.9 |
| | 103 | 444.3 | 16.0 | 2.4 | 4.5 | 161.3 | 13.5 | 26.0 | 0.8 |
| 14/0.5 | MC3 | 2037 | 19.3 | 26.8 | 5.7 | 8.5 | 1.2 | 2.2 | 4.4 |
| | 14 | 240.7 | 12.6 | 4.6 | 41.1 | 24.0 | 7.6 | 10.8 | 2.0 |
| | 14/8 | 9.5 | 6.0 | 0.7 | 7.3 | 4.6 | 5.8 | 4.7 | 3.0 |
| | 7 | 338.0 | 14.5 | 3.2 | 48.2 | 11.8 | 1.5 | 5.7 | 0.9 |
| | 8 | 257.0 | 6.4 | 3.6 | 46.9 | 10.4 | 4.0 | 9.8 | 2.2 |
| | 10 | 1175 | 16.2 | 3.7 | 15.1 | 10.2 | 4.0 | 6.5 | 2.8 |
| | 60 | 448.0 | 20.5 | 4.1 | 14.9 | 42.7 | 18.0 | 11.4 | 2.5 |
| | 61 | 157.3 | 33.8 | 5.0 | 11.1 | 109.9 | 51.6 | 16.6 | 1.0 |

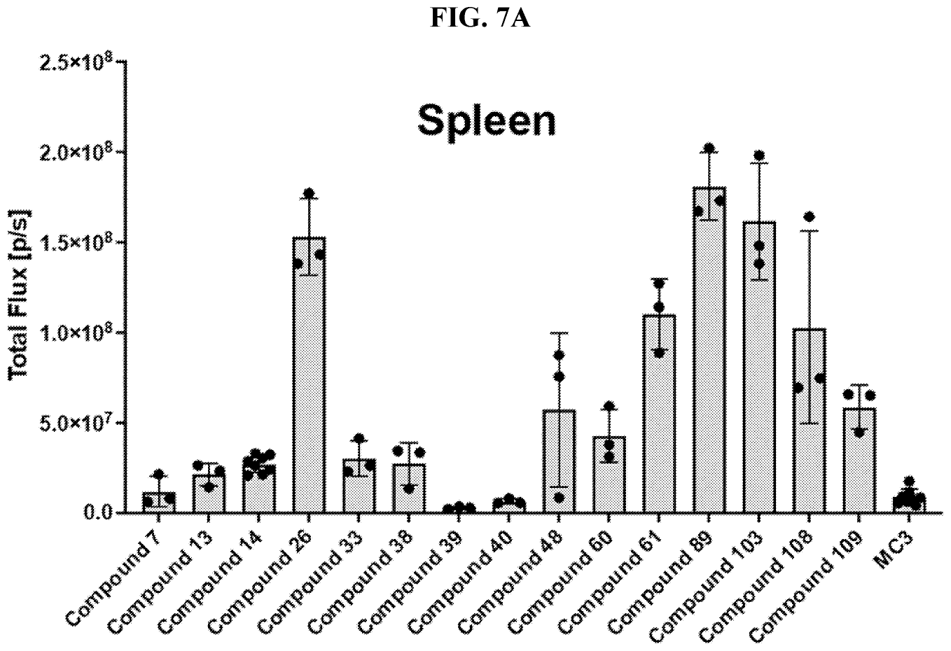

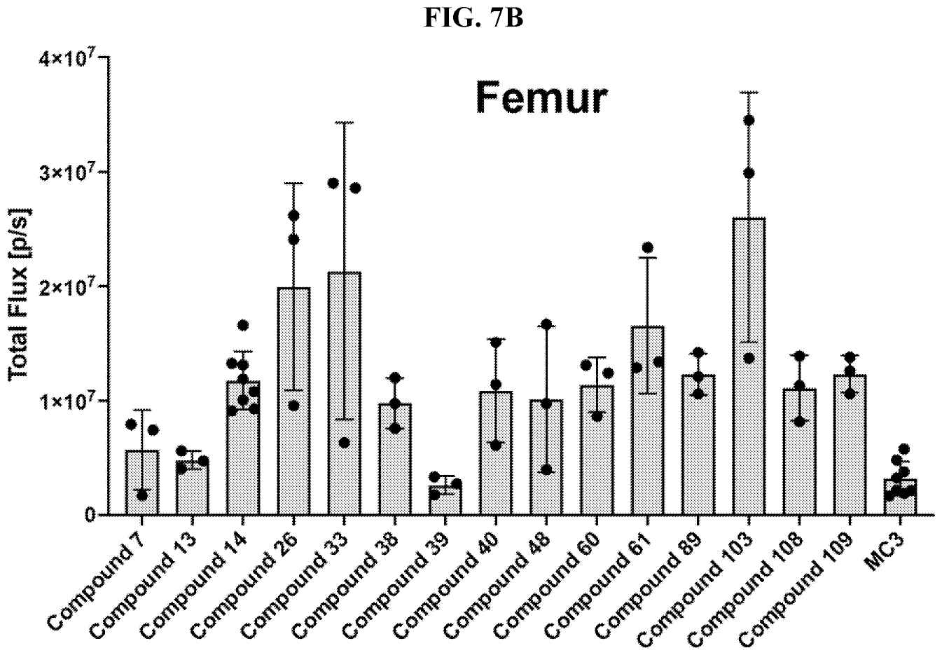

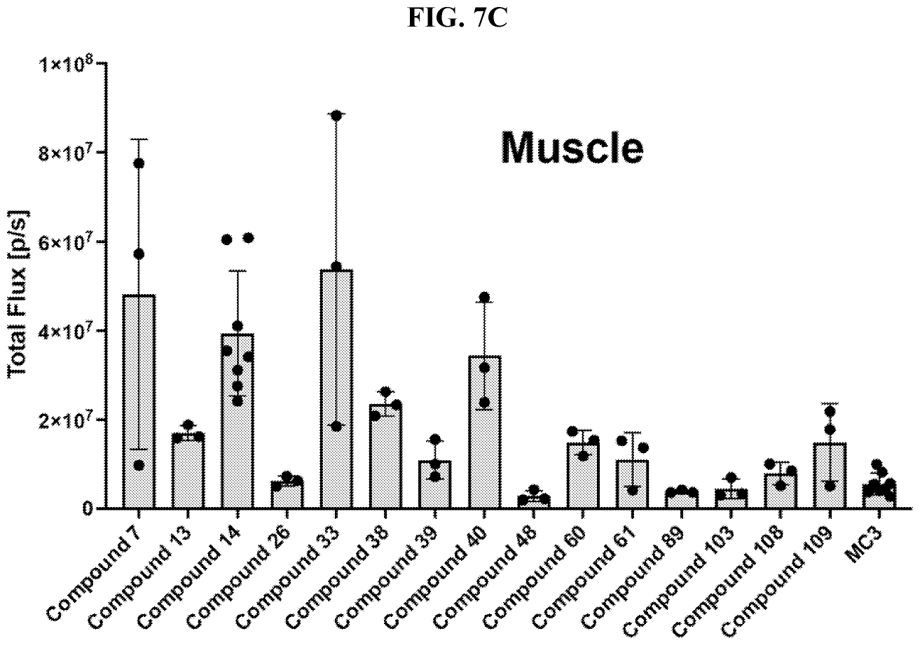

Additional screening results for Compounds 7, 13, 14, 26, 33, 38, 39, 40, 48, 60, 61, 89, 103, and 109 in spleen, femur, and muscle are shown in FIG. 7A, FIG. 7B, and FIG. 7C, with MC3 as a used as a control for comparison.

### Example 6—In Vivo Activity Screening in Non-Human Primates

Non-human primate (NIIP) biodistribution studies were performed in cynomolgus monkeys (e.g., Macaca fascicularis), aged 4-6 years. Animals were premedicated with dexamethasone, famotidine and diphenhydramine (at 1, 0.5 and 5 mg/kg, respectively) −2 and −1 day prior to test material dosing. LNP formulations (prepared in Formulation F2 from Example 1) were prepared with ionizable lipids so that each LNP formulation encapsulates one or more mRNAs with predetermined unique barcode sequences as their cargo. LNPs in PBS, identified by their unique barcoded mRNA cargo, are then pooled together at predetermined ratios. To evaluate LNP biodistribution into tissues upon systemic administration, NHPs received the dose administration by single intravenous infusion via peripheral vein (cephalic, saphenous, or another available) for 30 minutes with LNP dosing material (individual or pooled) in PBS at different total dose (0.5-1 mg/kg) levels.

At predetermined time points (4-6 hours after pooled test material administration), animals were sacrificed by exsanguination via the femoral artery after the intravenous injection of ketamine (10 mg/kg) and Euthasol® (0.25 mL/kg). Then, all animals underwent whole-body perfusion with PBS prior to tissue collection. 20-25 mg samples for biodistribution analysis were collected from all treatment group animals and then placed in sample tubes containing RNALater™ solution. All tissue samples were stored at room temperature for 24 hours, then the supernatants were removed, and samples were frozen.

To determine the unique LNPs distributed in the tissues, samples were homogenized by a tissue homogenizer (e.g., TissueLyser). Total RNA from the tissue homogenates were extracted by standard methods. Then, normalized amounts of RNA were sequenced using a targeted RNA sequencing approach with unique molecular identifiers (UMI) to ensure accurate RNA molecule counting. For each tissue, the resulting RNA molecule counts were normalized by pooled test article RNA counts. The results were analyzed using the internal bioinformatics pipeline.

LNP pool composition and UMI cound for multiple NHP organs are shown in Table 7. LNPs comprising MC3 used as a control and for cross-mouse comparison. Dose is in mg/kg; LC is Lipid Compound according to Table 1.

TABLE 7
in vivo Activity Screening

| | | Mean normalized UMI counts (×10⁴) | | | | | | | | |
|---|---|---|---|---|---|---|---|---|---|---|
| | | Bone | | | | | | | | |
| NHP | LC | Marrow | Brain | Heart | Kidney | Liver | Lung | Pancreas | Muscle | Spleen |
| 1 | 14 | 2.443 | 0.218 | 1.245 | 0.839 | 1.394 | N/A | 2.410 | 3.327 | 3.619 |
| | 14/8 | 7.095 | 1.239 | 3.649 | 4.948 | 5.569 | | 14.84 | 12.46 | 3.812 |
| | 17 | 0.410 | 0.033 | 0.216 | 0.140 | 0.563 | | 0.818 | 1.129 | 4.780 |
| | 50 | 0.450 | 0.199 | 0.576 | 0.386 | 0.316 | | 1.114 | 1.781 | 0.193 |
| | 51 | 0.824 | 0.113 | 1.049 | 0.355 | 1.782 | | 3.984 | 2.078 | 0.556 |
| | 52 | 1.615 | 0.326 | 1.474 | 0.790 | 1.577 | | 2.513 | 3.796 | 0.838 |
| | 53 | 1.979 | 0.293 | 1.351 | 1.132 | 2.308 | | 4.205 | 4.094 | 1.265 |
| | 54 | 0.710 | 0.112 | 0.776 | 0.356 | 1.521 | | 1.765 | 1.921 | 0.541 |
| | 55 | 0.723 | 0.106 | 0.781 | 0.335 | 1.541 | | 1.790 | 1.922 | 0.543 |
| | 56 | 0.624 | 0.095 | 0.804 | 0.306 | 1.726 | | 1.960 | 1.799 | 0.493 |
| | 57 | 0.293 | 0.056 | 0.474 | 0.176 | 1.510 | | 1.651 | 1.101 | 0.346 |
| | 58 | 1.038 | 0.095 | 1.235 | 0.420 | 1.770 | | 2.647 | 2.103 | 1.525 |
| | 60 | 4.186 | 0.354 | 1.130 | 1.606 | 3.164 | | 4.630 | 3.880 | 4.319 |
| | 61 | 6.205 | 0.369 | 1.064 | 1.795 | 4.765 | | 5.979 | 4.703 | 7.259 |
| | MC3 | 0.445 | 0.110 | 0.764 | 0.446 | 7.666 | | 2.643 | 2.024 | 2.690 |
| 2 | 13 | 207.1 | 64.0 | 129.1 | 142.6 | 102.3 | 124.6 | 84.3 | 130.1 | 181.2 |
| | 14 | 173.7 | 54.3 | 95.0 | 108.5 | 89.9 | 102.7 | 65.1 | 102.8 | 139.2 |
| | 15 | 75.3 | 26.6 | 126.2 | 102.1 | 49.3 | 91.0 | 39.8 | 72.5 | 38.5 |
| | 21 | 75.9 | 26.9 | 48.0 | 57.8 | 54.7 | 59.7 | 29.9 | 51.9 | 75.7 |
| | 23 | 76.1 | 29.2 | 105.3 | 107.1 | 83.4 | 94.7 | 34.8 | 70.2 | 66.2 |
| | 26 | 275.2 | 89.7 | 121.1 | 151.2 | 132.2 | 158.0 | 89.7 | 158.9 | 282.0 |
| | 27 | 68.9 | 28.1 | 117.2 | 115.6 | 99.6 | 92.3 | 32.8 | 74.8 | 58.2 |
| | 37 | 51.2 | 16.5 | 159.0 | 103.7 | 101.6 | 80.0 | 34.1 | 64.3 | 30.9 |
| | 39 | 86.6 | 29.7 | 188.5 | 139.1 | 74.6 | 114.7 | 49.8 | 87.6 | 43.6 |
| | 44 | 66.0 | 18.4 | 44.9 | 47.6 | 36.8 | 48.8 | 28.5 | 49.3 | 74.1 |
| | 45 | 79.7 | 24.5 | 112.2 | 120.6 | 72.4 | 93.6 | 39.0 | 76.9 | 67.8 |
| | 46 | 61.1 | 25.5 | 79.9 | 87.9 | 95.2 | 71.7 | 25.9 | 61.2 | 53.9 |
| | 48 | 249.3 | 81.9 | 103.7 | 133.6 | 118.9 | 141.0 | 73.2 | 140.2 | 267.4 |
| | 49 | 37.5 | 12.0 | 46.3 | 34.0 | 54.3 | 32.6 | 14.7 | 33.7 | 25.0 |
| | 59 | 58.0 | 14.1 | 68.3 | 38.8 | 55.0 | 37.2 | 163.6 | 82.4 | 26.3 |
| | 62 | 19.0 | 3.8 | 87.0 | 21.9 | 27.4 | 21.8 | 8.1 | 43.9 | 29.8 |
| | 63 | 233.0 | 72.8 | 115.8 | 153.6 | 108.2 | 139.5 | 89.4 | 142.7 | 207.6 |
| | 64 | 113.2 | 35.7 | 91.2 | 101.6 | 79.6 | 80.2 | 46.8 | 81.4 | 82.7 |
| | 66 | 114.8 | 34.3 | 102.7 | 106.1 | 82.8 | 83.5 | 46.1 | 87.6 | 90.5 |
| | 67 | 277.0 | 100.6 | 130.7 | 208.8 | 139.1 | 176.2 | 107.4 | 171.3 | 245.9 |
| | 73 | 270.2 | 82.6 | 117.8 | 209.9 | 140.9 | 176.7 | 104.1 | 165.9 | 221.0 |
| | 81 | 275.6 | 103.0 | 130.2 | 205.4 | 149.7 | 166.3 | 100.7 | 172.2 | 253.2 |
| | 91 | 296.1 | 108.2 | 117.6 | 195.7 | 146.0 | 178.3 | 101.4 | 177.6 | 271.5 |
| | 93 | 38.3 | 8.3 | 92.9 | 53.2 | 60.9 | 36.2 | 60.3 | 51.7 | 23.2 |
| | 96 | 153.5 | 48.0 | 103.6 | 106.3 | 81.0 | 92.5 | 72.3 | 102.7 | 106.2 |
| | 98 | 100.7 | 34.4 | 100.1 | 89.8 | 81.7 | 80.1 | 42.3 | 73.6 | 65.3 |
| | 99 | 295.5 | 100.1 | 127.6 | 228.1 | 151.2 | 183.5 | 110.0 | 183.7 | 253.9 |
| | 100 | 296.1 | 104.4 | 118.8 | 169.0 | 146.7 | 173.5 | 81.4 | 157.8 | 291.7 |
| | 101 | 288.2 | 109.7 | 112.2 | 168.8 | 144.4 | 171.8 | 79.4 | 161.3 | 285.2 |
| | 102 | 110.2 | 34.3 | 81.4 | 84.6 | 85.8 | 72.3 | 43.7 | 77.5 | 83.8 |
| | 103 | 311.4 | 116.8 | 129.1 | 210.8 | 162.9 | 185.5 | 100.3 | 187.2 | 297.1 |
| | MC3 | 31.7 | 6.8 | 70.2 | 25.7 | 328.6 | 21.3 | 23.6 | 35.6 | 63.0 |
| 3 | 7 | 210.0 | 95.7 | 181.6 | 146.6 | 106.7 | 193.4 | 119.5 | 107.8 | 206.0 |
| | 8 | 160.7 | 79.4 | 146.5 | 126.5 | 103.3 | 157.8 | 96.1 | 99.2 | 159.0 |
| | 10 | 128.7 | 65.6 | 121.9 | 99.7 | 81.4 | 127.1 | 83.7 | 74.2 | 130.3 |
| | 14 | 134.1 | 67.5 | 133.4 | 103.5 | 92.2 | 128.3 | 76.5 | 84.3 | 133.7 |
| | 14/8 | 149.2 | 96.8 | 161.4 | 147.6 | 137.5 | 183.1 | 99.2 | 113.0 | 95.4 |
| | 60 | 185.8 | 85.2 | 158.1 | 165.8 | 202.8 | 192.9 | 87.4 | 129.8 | 147.5 |
| | 61 | 202.0 | 68.6 | 136.0 | 147.2 | 271.4 | 177.4 | 66.8 | 126.0 | 192.8 |
| | 65 | 129.9 | 53.3 | 103.5 | 84.7 | 123.8 | 108.7 | 59.7 | 71.8 | 141.1 |
| | 80 | 197.2 | 47.5 | 98.3 | 120.8 | 263.7 | 148.4 | 52.7 | 98.3 | 190.1 |
| | 82 | 224.2 | 41.1 | 83.5 | 93.6 | 223.1 | 126.7 | 38.7 | 79.0 | 282.9 |
| | 87 | 212.6 | 58.9 | 115.3 | 123.9 | 252.3 | 157.0 | 54.9 | 100.1 | 225.4 |
| | 89 | 218.3 | 46.7 | 92.6 | 105.6 | 262.2 | 138.9 | 43.5 | 91.1 | 236.8 |
| | 105 | 86.6 | 60.1 | 99.5 | 83.5 | 80.3 | 105.4 | 55.9 | 60.8 | 68.5 |
| | 106 | 200.2 | 37.2 | 78.0 | 94.2 | 231.1 | 122.8 | 39.6 | 81.7 | 242.0 |
| | 108 | 201.2 | 85.1 | 158.2 | 147.8 | 172.7 | 180.1 | 82.7 | 114.8 | 174.9 |
| | 109 | 183.7 | 78.1 | 148.0 | 150.6 | 196.6 | 177.7 | 77.3 | 116.3 | 161.2 |
| | 110 | 102.0 | 69.3 | 120.8 | 102.9 | 105.3 | 119.2 | 68.6 | 81.1 | 84.2 |
| | MC3 | 41.9 | 16.6 | 34.3 | 32.9 | 289.5 | 34.6 | 26.4 | 35.5 | 109.7 |
| 4 | 14 | 2.323 | 1.176 | 1.995 | 1.899 | 2.050 | 1.980 | 0.692 | 1.880 | 2.291 |
| | 24 | 3.188 | 2.114 | 3.103 | 3.375 | 1.892 | 3.325 | 0.772 | 2.488 | 2.036 |
| | 25 | 1.750 | 0.707 | 1.534 | 1.488 | 1.207 | 1.465 | 0.920 | 1.390 | 2.010 |
| | 28 | 2.176 | 1.337 | 2.168 | 2.497 | 2.171 | 2.708 | 0.654 | 1.801 | 1.709 |
| | 29 | 2.624 | 1.263 | 2.288 | 2.285 | 2.231 | 2.451 | 0.780 | 1.903 | 2.531 |
| | 31 | 2.716 | 0.861 | 1.655 | 2.140 | 3.066 | 2.661 | 0.764 | 1.576 | 2.870 |
| | 33 | 3.608 | 1.726 | 3.106 | 3.628 | 3.545 | 3.916 | 1.215 | 2.875 | 3.160 |
| | 34 | 2.608 | 1.456 | 2.520 | 2.604 | 2.098 | 2.617 | 0.935 | 2.070 | 2.364 |
| | 35 | 3.203 | 1.924 | 3.206 | 3.552 | 2.366 | 3.385 | 0.810 | 2.735 | 2.104 |
| | 36 | 2.919 | 2.072 | 2.868 | 3.045 | 1.011 | 2.732 | 0.584 | 2.251 | 1.021 |
| | 38 | 3.967 | 1.608 | 3.743 | 2.619 | 1.585 | 1.893 | 1.859 | 2.325 | 1.234 |
| | 40 | 2.777 | 1.224 | 3.122 | 2.103 | 1.912 | 1.430 | 1.925 | 1.866 | 0.912 |
| | 41 | 3.421 | 2.410 | 3.524 | 3.191 | 2.257 | 2.806 | 0.723 | 2.612 | 0.895 |
| | 42 | 2.575 | 1.129 | 3.001 | 1.985 | 2.857 | 1.450 | 1.742 | 1.959 | 0.975 |
| | 43 | 2.200 | 1.188 | 1.970 | 1.991 | 1.421 | 1.903 | 0.585 | 1.724 | 1.437 |
| | 47 | 2.005 | 1.103 | 1.903 | 1.916 | 1.361 | 1.774 | 0.560 | 1.640 | 1.394 |
| | MC3 | 0.675 | 0.255 | 1.876 | 0.680 | 8.589 | 0.581 | 0.748 | 1.072 | 3.059 |

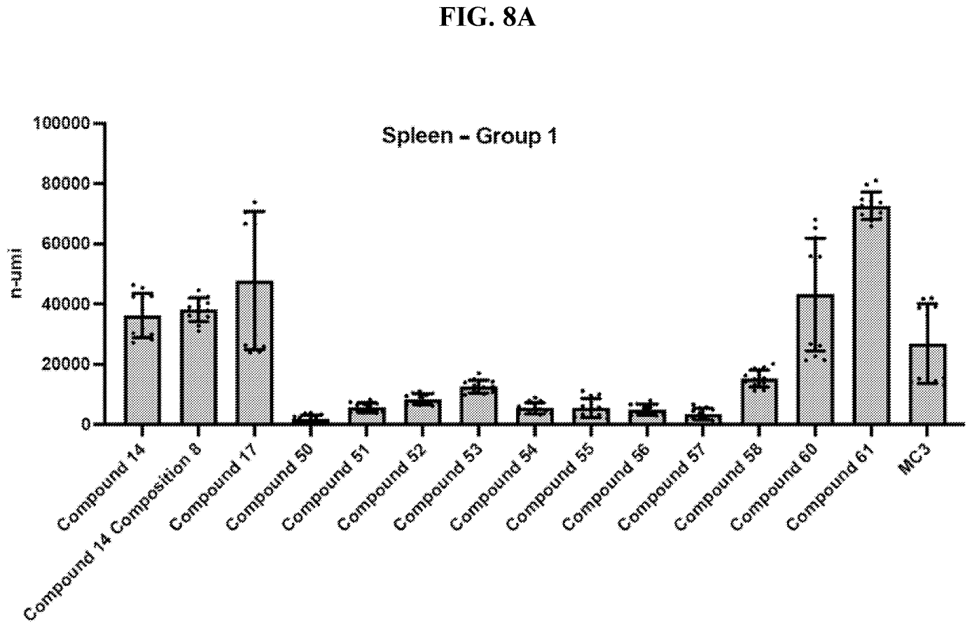

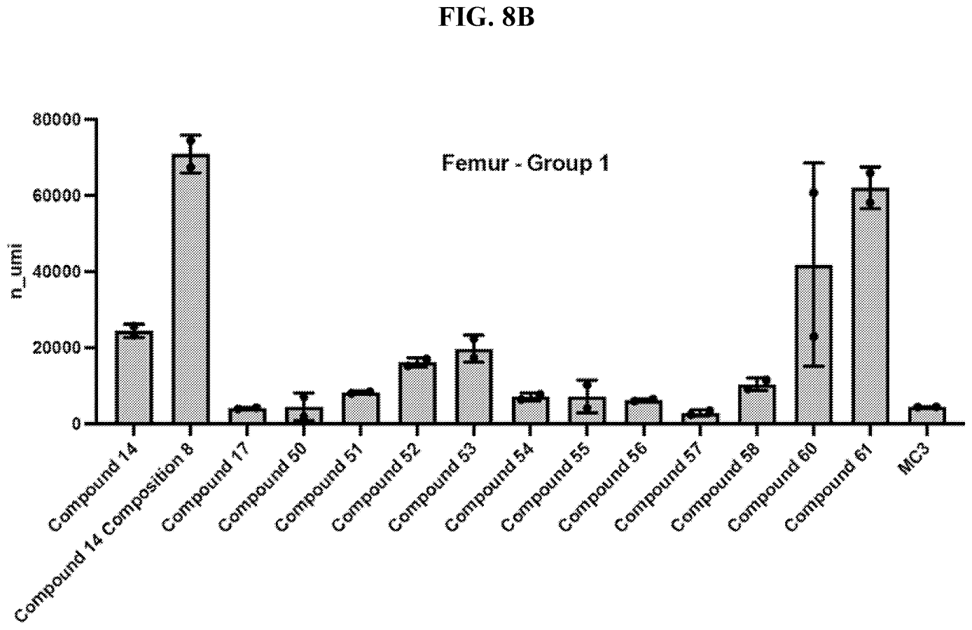

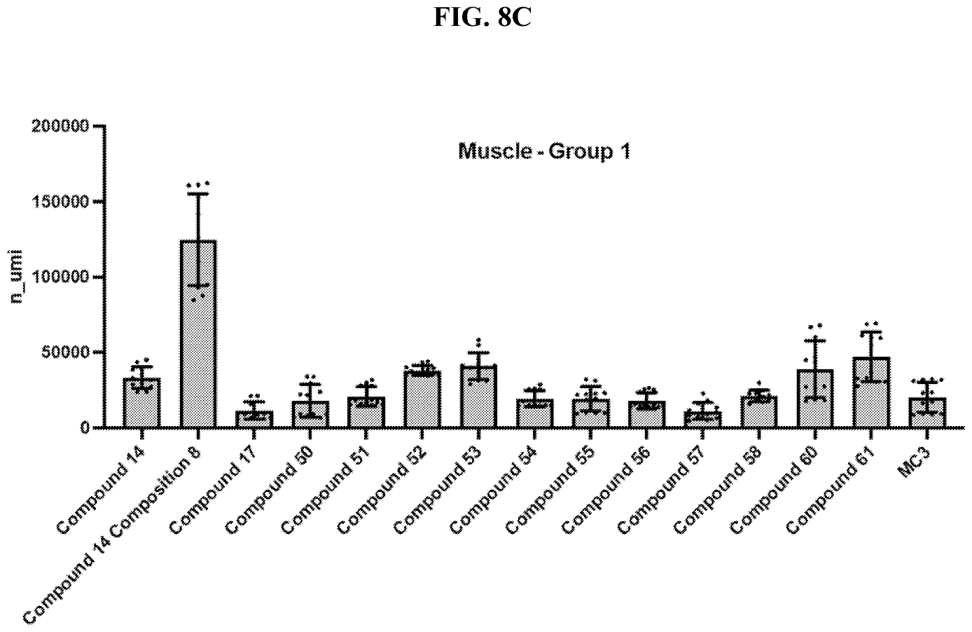

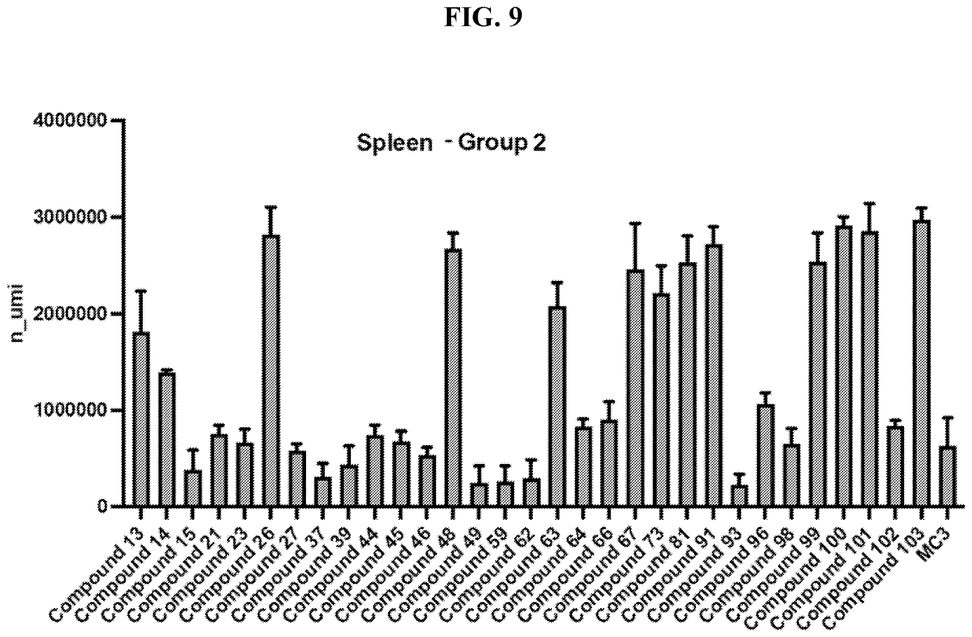

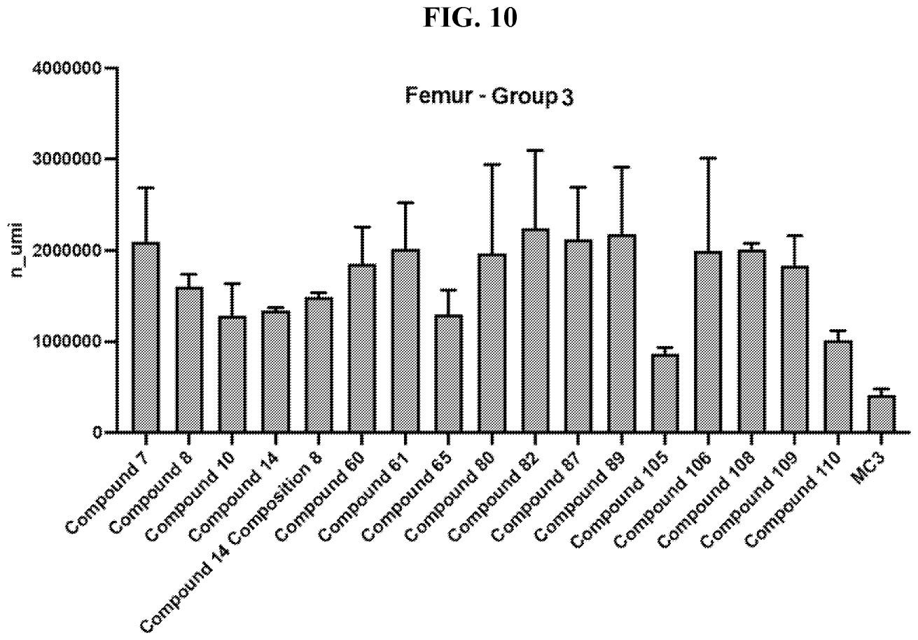

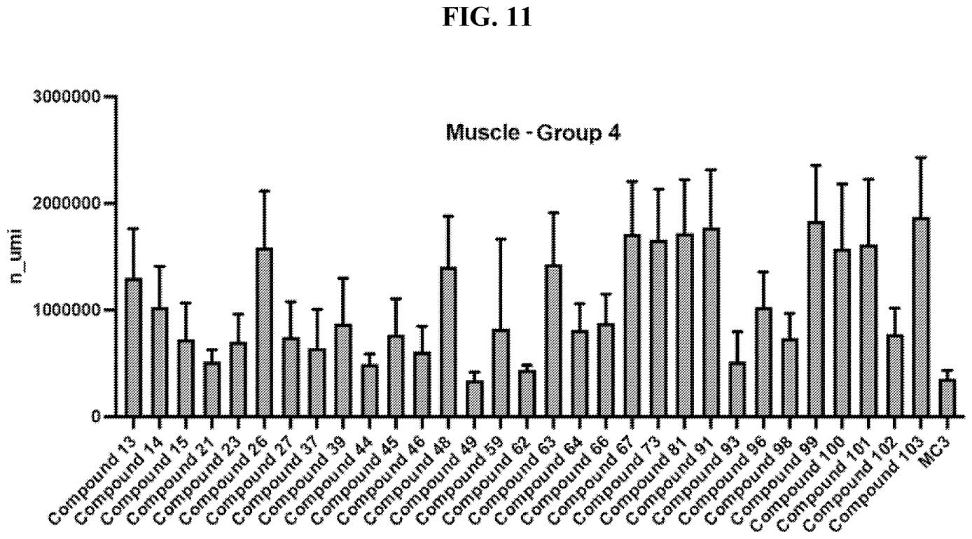

Screening results for NHP Group 1 (spleen, femur, and muscle) are shown in FIG. 8A, FIG. 8B, and FIG. 8C. Screening results for NHP Group 2 (spleen) are shown in FIG. 9. Screening results for NHP Group 3 (femur) are shown in FIG. 10. Screening results for NHP Group 4 (muscle) are shown in FIG. 11.

### Example 7—General Materials and Methods for Syntheses

All temperatures are in degrees Celsius and are uncorrected. Reagent grade chemicals and anhydrous solvents were purchased from commercial sources and unless otherwise mentioned, were used without further purification. The names of the products were determined using the naming software included in the Biovia electronic lab notebook. Silica gel chromatography was performed on Teledyne Isco instruments using pre-packaged disposable SiO₂ stationary phase columns with eluent flow-rate ranges of 15 to 200 mL/min, UV detection (254 and 280 nm). Reverse phase purification was carried out using C₁₈ columns, UV detection (214 and 254 nm). The chemical shifts are reported in parts-per-million and are referenced to solvent peaks, which in ¹H NMR appear at 7.26 ppm for CDCl₃, 2.50 for DMSO-d₆, and 3.31 ppm for CD₃OD.

### Terms and Abbreviations:

4-PPY 4-pyrrolidin-1-ylpyridine;
Ac acetyl;
aq aqueous;
Bn benzyl;
DCM dichloromethane;
DIPEA N,N-diisopropylethylamine;
DMAP 4-dimethylaminopyridine;
EDCI 1-ethyl-3-(3-dimethylaminopropyl)carbodiimide hydrochloride;
Et ethyl;
EtOAc ethyl acetate;
h hour(s);
HCl hydrochloride;
HPLC high performance liquid chromatography;
LCMS liquid chromatography mass spectrometry;
mCPBA 3-chloroperbenzoic acid;
Me methyl;
min minute(s);
NaHCO₃ sodium bicarbonate;
Na₂SO₄ sodium sulfate;
PPTS pyridinium p-toluenesulfonate;
Py pyridine;
TEA triethylamine;
TEMPO 1-oxidanyl-2,2,6,6-tetramethyl-piperidine;
NH₄Cl ammonium chloride;
NMR nuclear magnetic resonance;
p-TsOH—H₂O para-toluenesulfonic acid monohydrate;
sat. saturated;
TBS tert-butyldimethylsilyl;
THF tetrahydrofuran.

### Example 8—Compound 1

### 2-((oleoyloxy)methyl)-2-(((4-(((2-(pyrrolidin-1-yl)ethyl)carbamoyl)oxy)decanoyl)oxy)methyl)propane-1,3-diyl dioleate

### Step 1:

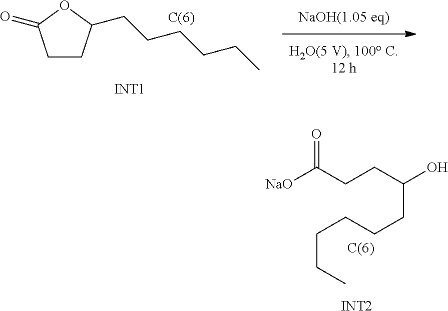

To a solution of 5-hexyltetrahydrofuran-2-one (100 g, 587.38 mmol, 1 eq) in H₂O (500 mL) was added NaOH (24.67 g, 616.75 mmol, 1.05 eq) slowly. The mixture was stirred at 100° C. for 12 hr under N₂. The reaction mixture was concentrated under reduced pressure to remove solvent. Compound 4-hydroxydecanoyloxysodium (90 g, crude) was obtained as a white solid and it was used in next step directly.

### Step 2:

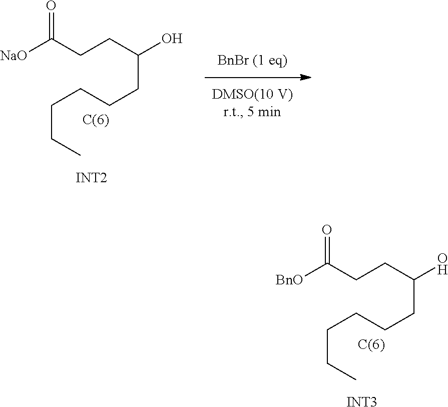

To a solution of 4-hydroxydecanoyloxysodium (10 g, 47.56 mmol, 1 eq) in DMSO (100 mL) was added bromomethylbenzene (8.13 g, 47.56 mmol, 5.65 mL, 1 eq) dropwise. The mixture was stirred at 25° C. for 5 min under N₂. The reaction mixture was diluted with sat. NaCl (100 mL) and extracted with EtOAc (200 mL) (100 mL*2). The combined organic layers were dried over Na₂SO₄, filtered and the filtrate was concentrated under reduced pressure to give a residue. Compound benzyl 4-hydroxydecanoate (12 g, crude) was obtained as a pale-yellow oil and it was used in next step quickly.

### Step 3:

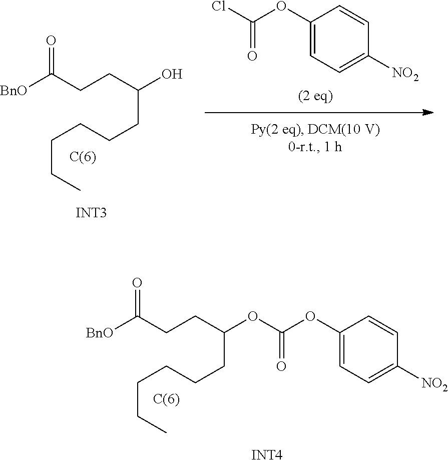

To a solution of benzyl 4-hydroxydecanoate (12 g, 43.11 mmol, 1 eq) in DCM (120 mL) was added (4-nitrophenyl) carbonochloridate (17.38 g, 86.21 mmol, 2 eq) and Py. (6.82 g, 86.21 mmol, 6.96 mL, 2 eq) slowly at 0° C. The mixture was stirred at 25° C. for 1 hr under N₂. The reaction mixture was diluted with petroleum ether (100 mL), filtered and the filtrate was concentrated under reduced pressure to give a residue. The residue was purified by column chromatography (SiO₂, Petroleum ether/Ethyl acetate=100/1 to 10/1). Compound benzyl 4-(4-nitrophenoxy)carbonyloxydecanoate (10 g, 22.55 mmol, 52.31% yield) was obtained as a colorless oil. ¹H NMR (400 MHz, CDCl₃) δ ppm 8.30-8.25 (m, 2H) 7.37-7.27 (m, 7H) 5.14 (s, 2H) 4.91-4.85 (m, 1H) 2.53-2.50 (t, J=7.6 Hz, 2H) 2.16-2.07 (m, 1H) 2.05-1.97 (m, 1H) 1.77-1.72 (m, 1H) 1.67-1.60 (m, 1H) 1.43-1.30 (m, 8H) 0.91-0.88 (t, J=6.0 Hz, 3H).

### Step 4:

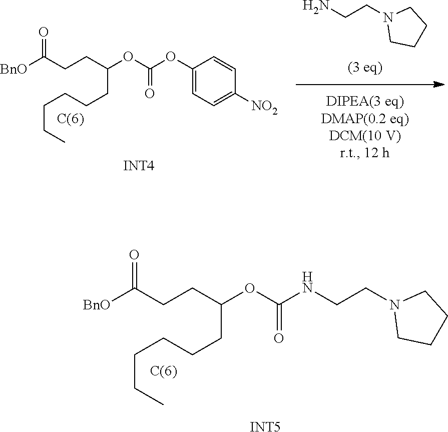 [SMILES: CCCCCCC(CCC(=O)OCc1ccccc1)OC(=O)Oc1ccc([N+](=O)[O-])cc1 ; "INT4" ; NCCN1CCCC1 ; CCCCCCC(CCC(=O)OCc1ccccc1)OC(=O)NCCN1CCCC1 ; "INT5"]

To a solution of benzyl 4-(4-nitrophenoxy)carbonyloxydecanoate (10 g, 22.55 mmol, 1 eq) in DCM (100 mL) was added 2-pyrrolidin-1-ylethanamine (7.72 g, 67.65 mmol, 3 eq), DMAP (550.94 mg, 4.51 mmol, 0.2 eq) and DIPEA (8.74 g, 67.65 mmol, 11.78 mL, 3 eq). The mixture was stirred at 25° C. for 12 hr under N₂. The reaction mixture was diluted with H₂O (100 mL) and extracted with DCM 200 mL (100 mL*2). The combined organic layers were concentrated under reduced pressure to give a residue. The residue was purified by column chromatography (SiO₂, Petroleum ether/Ethyl acetate=50/1 to 0/1). Compound benzyl 4-(2-pyrrolidin-1-ylethylcarbamoyloxy) decanoate (7 g, 16.72 mmol, 74.17% yield) was obtained as a colorless oil.

### Step 5:

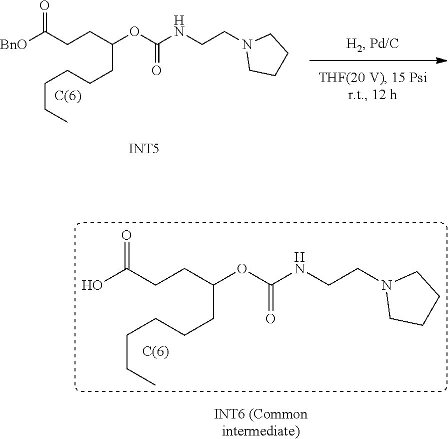

To a suspension of Pd/C (2 g, 1.88 mmol, 10% purity, 0.112 eq) in THF (140 mL) was added benzyl 4-(2-pyrrolidin-1-ylethylcarbamoyloxy)decanoate (7 g, 16.72 mmol, 1 eq). The mixture was stirred at 25° C. for 12 hr under H2 (15 Psi). The reaction mixture was filtered and the filtrate was concentrated under reduced pressure to give a residue. The residue was purified by column chromatography (SiO2, DCM/MeOH=50/1 to 3/1). Compound 4-(2-pyrrolidin-1-ylethylcarbamoyloxy)decanoic acid (3 g, 9.13 mmol, 54.62% yield) was obtained as a colorless oil. ¹H NMR (400 MHz, CDCl₃) δ ppm 10.23 (s, 1H) 6.27 (s, 1H) 4.77-4.71 (m, 1H) 3.68-3.60 (m, 1H) 3.19-3.14 (m, 1H) 3.08-2.95 (m, 5H) 2.82- 2.77 (m, 1H) 2.37-2.21 (m, 2H) 2.06-2.00 (m, 1H) 1.96 (s, 4H) 1.84-1.74 (m, 1H) 1.64-1.58 (m, 1H) 1.52-1.45 (m, 1H) 1.30-1.26 (m, 8H) 0.88-0.85 (t, J=6.8 Hz, 3H).

### Step 6:

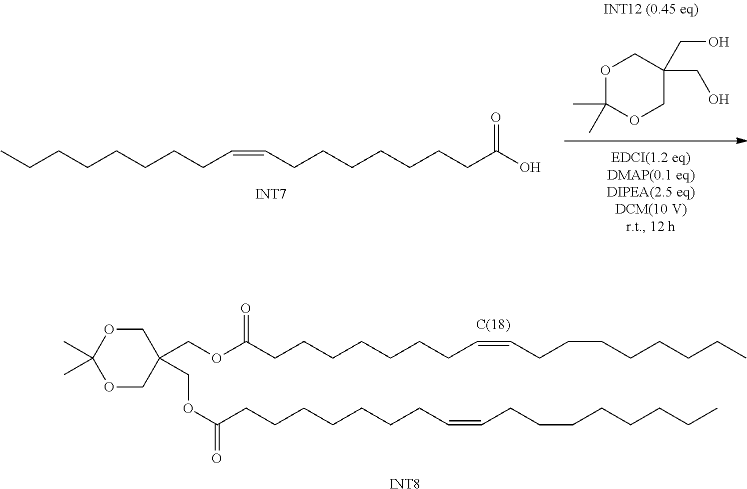

A mixture of [5-(hydroxymethyl)-2,2-dimethyl-1,3-dioxan-5-yl]methanol (8 g, 45.40 mmol, 0.45 eq), (Z)-octadec-9-enoic acid (28.50 g, 100.89 mmol, 28.50 mL, 1 eq), EDCI (23.21 g, 121.07 mmol, 1.2 eq), DIPEA (32.60 g, 252.22 mmol, 43.93 mL, 2.5 eq) and DMAP (1.23 g, 10.09 mmol, 0.1 eq) in DCM (285 mL) was degassed and purged with N₂ for 3 times, and then the mixture was stirred at 20° C. for 12 hr under N₂ atmosphere. The reaction mixture was diluted with water (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound [2,2-dimethyl-5-[[(Z)-octadec-9-enoyl]oxymethyl]-1,3-dioxan-5-yl]methyl (Z)-octadec-9-enoate (20 g, 28.36 mmol, 56.23% yield) was obtained as a colorless oil. ¹H NMR (400 MHz, CDCl₃) δ ppm 5.37-5.34 (m, 4H) 4.11 (s, 4H) 3.75 (s, 4H) 2.34-2.30 (t, J=7.6 Hz, 4H) 2.05-1.99 (m, 8H) 1.63-1.60 (m, 4H) 1.43 (s, 6H) 1.31-1.27 (m, 40H) 0.91-0.87 (t, J=6.8 Hz, 6H).

### Step 7:

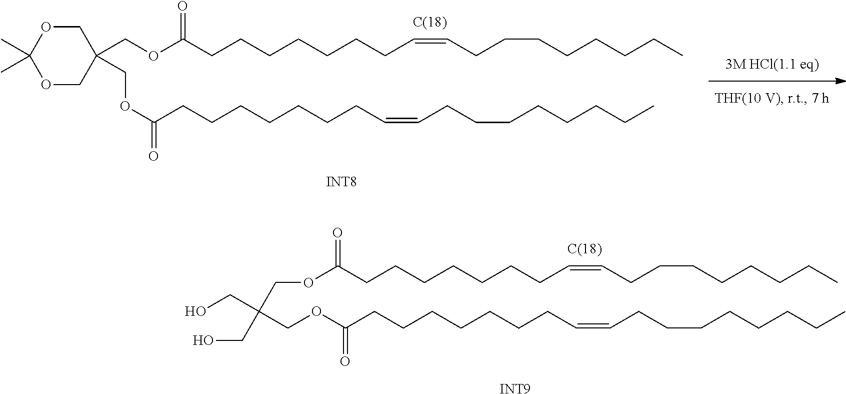

To a solution of [2,2-dimethyl-5-[[(Z)-octadec-9-enoyl]oxymethyl]-1,3-dioxan-5-yl]methyl (Z)-octadec-9-enoate (20 g, 28.36 mmol, 1 eq) in THF (200 mL) was added HCl (3 M, 10.40 mL, 1.1 eq) slowly at 0° C. Then the mixture was stirred at 20° C. for 12 hr under N₂ atmosphere. The reaction mixture was adjusted pH=7 with sat. Na₂CO₃ at 0° C. and it was extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=20/1 to 1/1). Compound [2,2-bis(hydroxymethyl)-3-[(Z)-octadec-9-enoyl]oxy-propyl](Z)-octadec-9-enoate (10 g, 15.04 mmol, 53.01% yield) was obtained as a colorless oil. ¹H NMR (400 MHz, CDCl₃) δ ppm 5.38-5.34 (m, 4H) 4.15 (s, 4H) 3.58 (s, 4H) 2.73 (s, 2H) 2.38-2.34 (t, J=7.2 Hz, 4H) 2.07-2.00 (m, 8H) 1.65-1.60 (m, 4H) 1.31-1.25 (m, 40H) 0.91-0.87 (t, J=6.8 Hz, 6H).

### Step 8:

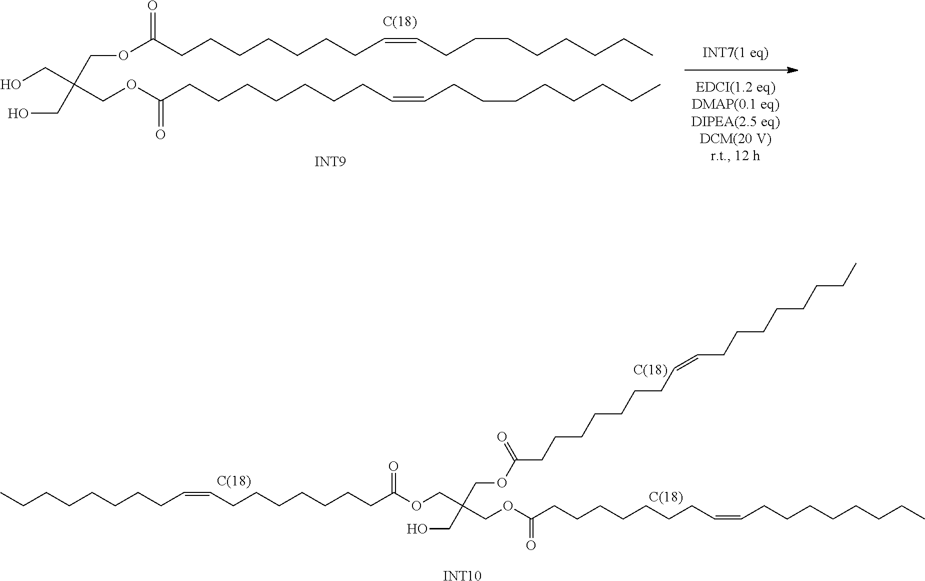

To a solution of [2,2-bis(hydroxymethyl)-3-[(Z)-octadec-9-enoyl]oxy-propyl](Z)-octadec-9-enoate (4 g, 6.01 mmol, 1 eq), EDCI (1.38 g, 7.22 mmol, 1.2 eq), DMAP (73.48 mg, 601.47 mol, 0.1 eq) and DIPEA (1.94 g, 15.04 mmol, 2.62 mL, 2.5 eq) in DCM (40 mL) was added oleic acid (1.70 g, 6.01 mmol, 1.70 mL, 1 eq) in DCM (40 mL) slowly. The reaction mixture was stirred at 20° C. for 12 hr under N₂ atmosphere. The reaction mixture was diluted with water (100 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound [2-(hydroxymethyl)-3-[(Z)-octadec-9-enoyl]oxy-2-[[(Z)-octadec-9-enoyl]oxymethyl]propyl](Z)-octadec-9-enoate (1.5 g, 1.61 mmol, 26.83% yield) was obtained as a colorless oil. ¹H NMR (400 MHz, CDCl₃) δ ppm 5.40-5.31 (m, 6H) 4.12 (s, 6H) 3.50-3.49 (d, J=6.8 Hz, 2 H) 2.53-2.49 (t, J=7.2 Hz, 1H) 2.35-2.31 (t, J=7.2 Hz, 6H) 2.07-2.00 (m, 12H) 1.64-1.58 (m, 6H) 1.31-1.28 (m, 60H) 0.90-0.87 (t, J=6.8 Hz, 9H).

### Step 9:

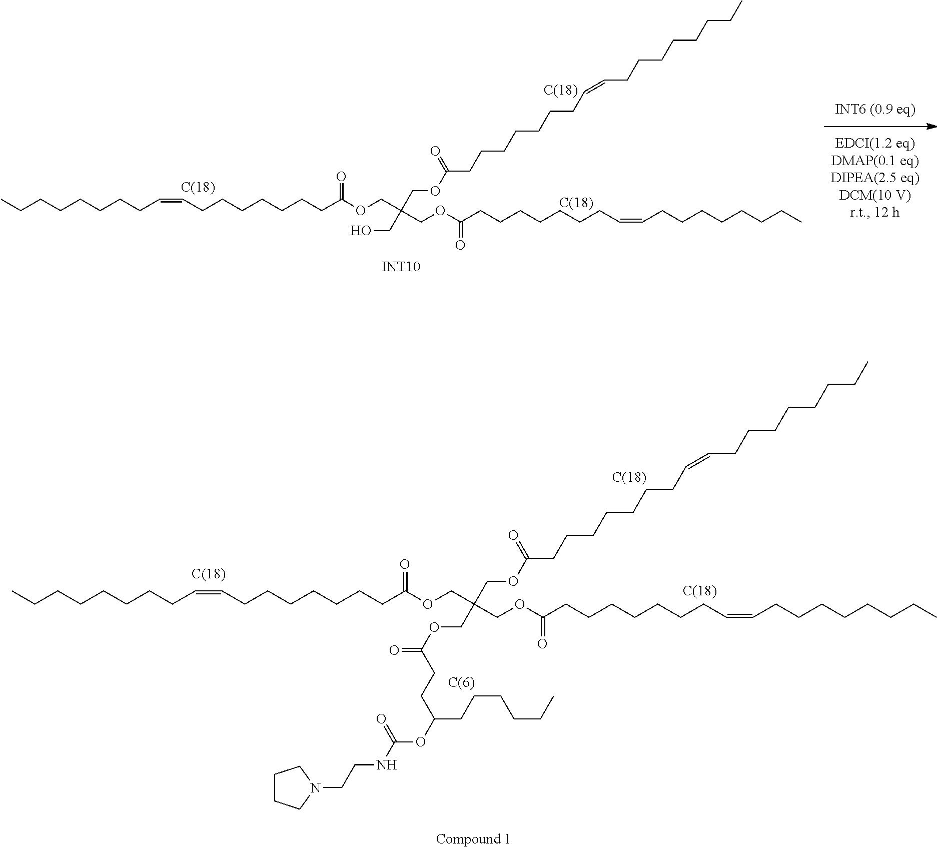

To a mixture of 4-(2-pyrrolidin-1-ylethylcarbamoyloxy)decanoic acid (477.04 mg, 1.45 mmol, 0.9 eq), EDCI (371.24 mg, 1.94 mmol, 1.2 eq), DMAP (19.72 mg, 161.38 mol, 0.1 eq) and DIPEA (521.43 mg, 4.03 mmol, 702.74 μL, 2.5 eq) in DCM (15 mL) was added [2-(hydroxymethyl)-3-[(Z)-octadec-9-enoyl]oxy-2-[[(Z)-octadec-9-enoyl]oxymethyl]propyl](Z)-octadec-9-enoate (1.5 g, 1.61 mmol, 1 eq) slowly. The mixture was stirred at 25° C. for 12 hr under N₂ atmosphere. The reaction mixture was diluted with water (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 0/1). Compound [2,2-bis[[(Z)-octadec-9-enoyl]oxymethyl]-3-[4-(2-pyrrolidin-1-ylethyl carbamoyloxy)decanoyloxy]propyl](Z)-octadec-9-enoate (104 mg, 83.88 mol, 5.20% yield) was obtained as a colorless oil. ¹H NMR (400 MHz, CDCl₃) δ ppm 5.80 (s, 1H) 5.39-5.32 (m, 6H) 4.75-4.71 (m, 1H) 4.17-4.08 (m, 8H) 3.52-3.50 (m, 2H) 3.20-2.94 (m, 4H) 2.39-2.35 (m, 2H) 2.33-2.29 (t, J=7.6 Hz, 6H) 2.02-1.99 (m, 14H) 1.94-1.86 (m, 2H) 1.83-1.75 (m, 2H) 1.61-1.58 (m, 8H) 1.49-1.42 (m, 2H) 1.30-1.27 (m, 68H) 0.90-0.87 (m, 12H).

### Example 9—Compound 2

### 2-((((9Z,12Z)-octadeca-9,12-dienoyl)oxy)methyl)-2-(((4-(((2-(pyrrolidin-1-yl)ethyl)carbamoyl)oxy)decanoyl)oxy)methyl)propane-1,3-diyl (9Z,9′Z,12Z,12′Z)-bis(octadeca-9,12-dienoate)

### Step 1:

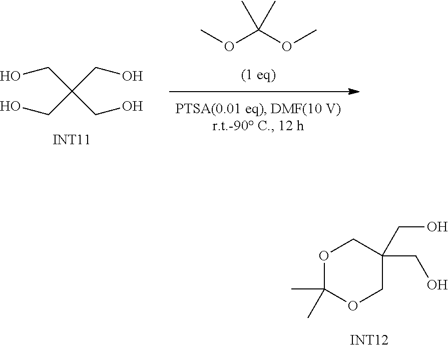

2,2-bis(hydroxymethyl) propane-1,3-diol (50 g, 367.25 mmol, 1 eq) was dissolved in DMF (500 mL) under N₂ in a 1000 mL single-necked round bottom flask. The mixture was stirred at 90° C. for 1 h under N₂. Then 2,2-dimethoxypropane (38.25 g, 367.25 mmol, 45.00 mL, 1 eq) and 4-methylbenzenesulfonic acid; hydrate (698.58 mg, 3.67 mmol, 0.01 eq) was added to the above reaction mixture at 70° C. under N₂. The mixture was stirred at 25° C. for 12 hr under N₂. The reaction mixture was quenched by using TEA (10 mL) and stirred at 25° C. for 1 hr under N₂. Then the mixture was concentrated under reduced pressure to give a residue. The residue was purified by column chromatography (SiO₂, Petroleum ether/Ethyl acetate=100/1 to 0/1). Compound [5-(hydroxymethyl)-2,2-dimethyl-1,3-dioxan-5-yl]methanol (28 g, 158.90 mmol, 43.27% yield) was obtained as a white solid.

### Step 2:

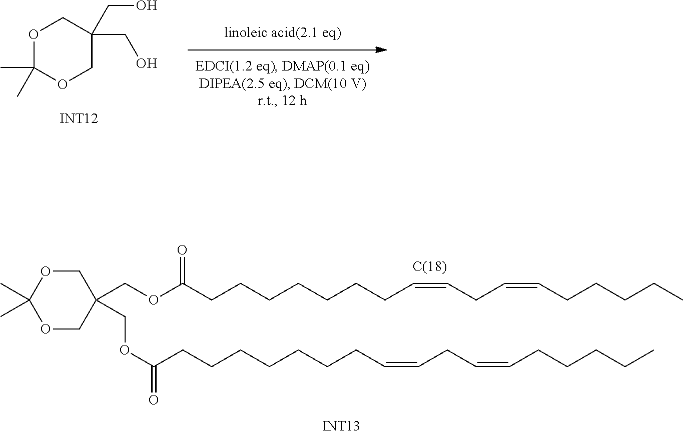

To a mixture of [5-(hydroxymethyl)-2,2-dimethyl-1,3-dioxan-5-yl]methanol (8.47 g, 48.04 mmol, 1 eq) and (9Z,12Z)-octadeca-9,12-dienoic acid (28.29 g, 100.89 mmol, 28.29 mL, 2.1 eq) in DCM (280 mL) was added EDCI (11.05 g, 57.65 mmol, 1.2 eq), DMAP (586.93 mg, 4.80 mmol, 0.1 eq) and DIPEA (15.52 g, 120.11 mmol, 20.92 mL, 2.5 eq). The mixture was stirred at 25° C. for 12 hr under N₂ atmosphere. The reaction mixture was diluted with H₂O (500 mL) and extracted with ethyl acetate 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2,2-dimethyl-5-[[(9Z,12Z)-octadeca-9,12-dienoyl]oxymethyl]-1,3-dioxan-5-yl]methyl (9Z,12Z)-octadeca-9,12-dienoate (15 g, 21.40 mmol, 44.54% yield) was obtained as a colorless oil. ¹H NMR (400 MHz, CDCl₃) δ ppm 11.79-11.42 (m, 1H), 5.40-5.36 (m, 4H), 2.80-2.77 (t, J=6.32 Hz, 2H), 2.38-2.40 (t, J=7.52 Hz, 2H), 2.08-2.04 (q, J=6.6 Hz, 4H), 1.66-1.61 (m, 2H), 1.37-1.31 (m, 14H), 0.92-0.88 (t, J=6.72 Hz, 3H).

### Step 3:

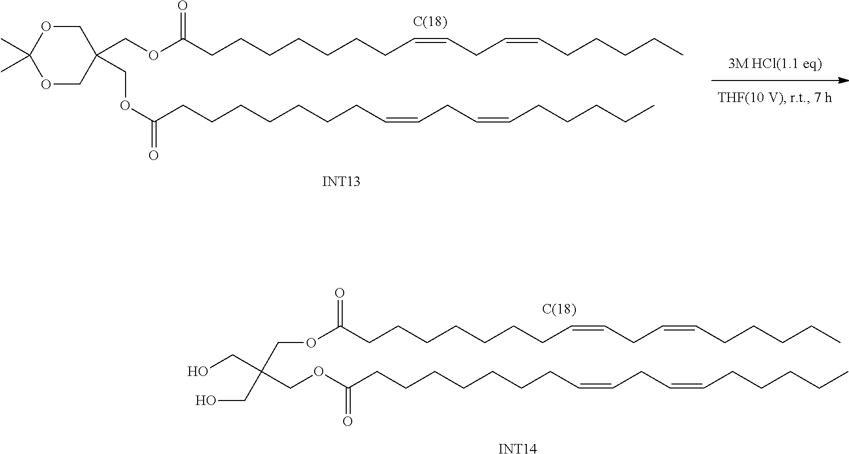

To a solution of [2,2-dimethyl-5-[[(9Z,12Z)-octadeca-9,12-dienoyl]oxymethyl]-1,3-dioxan-5-yl]methyl (9Z,12Z)-octadeca-9,12-dienoate (15 g, 21.40 mmol, 1 eq) in THE (150 mL) was added HCl (3 M, 7.85 mL, 1.1 eq). The mixture was stirred at 25° C. for 7 hr. The reaction mixture was diluted with H₂O 200 mL and extracted with Ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2,2-bis(hydroxymethyl)-3-[(9Z,12Z)-octadeca-9,12-dienoyl]oxy-propyl](9Z,12Z)-octadeca-9,12-dienoate (8 g, 12.10 mmol, 56.57% yield) was obtained as a colorless oil. ¹H NMR (400 MHz, CDCl₃) δ ppm 5.33-5.26 (m, 8H) 4.07 (s, 4H) 3.50 (s, 4H) 2.72-2.69 (t, J=6.8 Hz, 4H) 2.30-2.26 (t, J=7.2 Hz, 4H) 2.01-1.96 (m, 8H) 1.58-1.54 (t, J=7.2 Hz, 4H) 1.32-1.19 (m, 30H) 0.84-0.80 (t, J=6.8 Hz, 6H).

### Step 4:

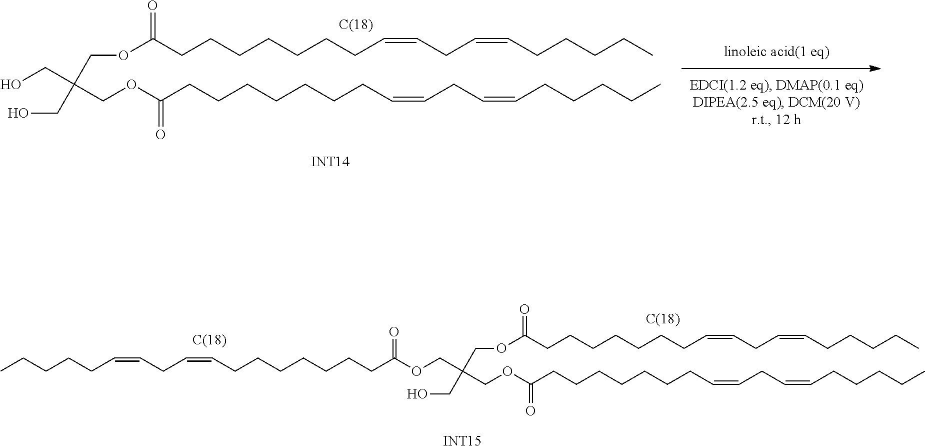

To a mixture of (9Z,12Z)-octadeca-9,12-dienoic acid (424.27 mg, 1.51 mmol, 424.27 μL, 0.5 eq) and [2,2-bis(hydroxymethyl)-3-[(9Z,12Z)-octadeca-9,12-dienoyl]oxy-propyl](9Z,12Z)-octadeca-9,12-dienoate (2 g, 3.03 mmol, 1 eq) in DCM (204 mL) was added DIPEA (977.63 mg, 7.56 mmol, 1.32 mL, 2.5 eq), EDCI (696.04 mg, 3.63 mmol, 1.2 eq) and DMAP (36.96 mg, 302.57 mol, 0.1 eq). The mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2-(hydroxymethyl)-3-[(9Z,12Z)-octadeca-9,12-dienoyl]oxy-2-[[(9Z,12Z)-octadeca-9,12-dienoyl]oxymethyl]propyl](9Z,12Z)-octadeca-9,12-dienoate (700 mg, 758.04 mol, 25.05% yield) was obtained as a colorless oil. ¹H NMR (400 MHz, CDCl₃) δ ppm 5.40-5.30 (m, 12H) 4.12 (s, 6H) 3.50-3.49 (d, J=6.8 Hz, 2H) 2.79-2.76 (t, J=6.4 Hz, 6H) 2.54-2.50 (t, J=7.2 Hz, 1H) 2.35-2.31 (t, J=7.6 Hz, 6H) 2.08-2.03 (m, 12H) 1.64-1.60 (m, 6H) 1.38-1.28 (m, 42H) 0.91-0.88 (t, J=6.8 Hz, 9H).

### Step 5:

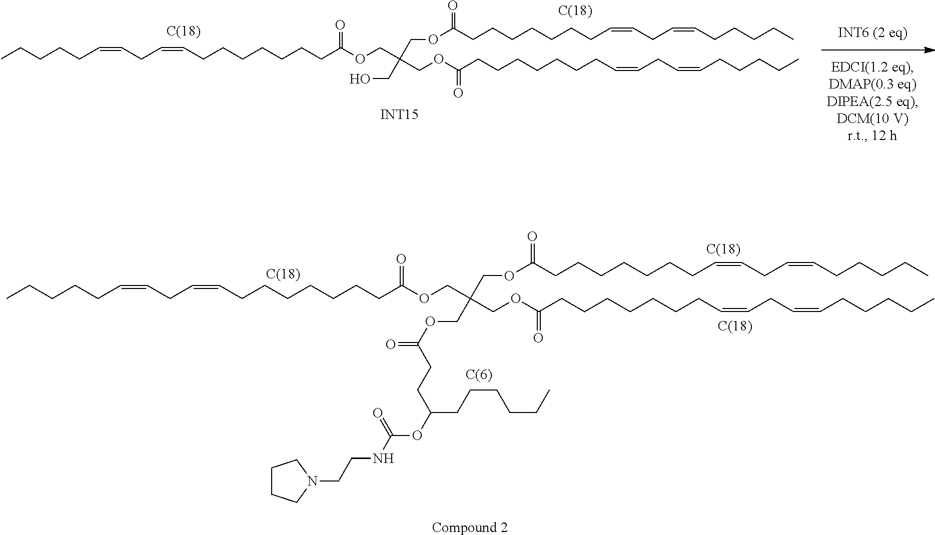

To a mixture of [2-(hydroxymethyl)-3-[(9Z,12Z)-octadeca-9,12-dienoyl]oxy-2-[[(9Z,12Z)-octadeca-9,12-dienoyl]oxymethyl]propyl](9Z,12Z)-octadeca-9,12-dienoate (700 mg, 758.04 mol, 1 eq) and 4-(2-pyrrolidin-1-ylethylcarbamoyloxy)decanoic acid (497.95 mg, 1.52 mmol, 2 eq) in DCM (7 mL) was added DMAP (27.78 mg, 227.41 mol, 0.3 eq), DIPEA (244.93 mg, 1.90 mmol, 330.09 μL, 2.5 eq) and EDCI (174.38 mg, 909.65 mol, 1.2 eq). The mixture was stirred at 25° C. for 12 hr under N₂ atmosphere. The reaction mixture was diluted with H₂O (30 mL) and extracted with ethyl acetate 30 mL (10 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2,2-bis[[(9Z,12Z)-octadeca-9,12-dienoyl]oxymethyl]-3-[4-(2-pyrrolidin-1-ylethylcarbamoyloxy)decanoyloxy]propyl](9Z,12Z)-octadeca-9,12-dienoate (110 mg, 88.24 mol, 11.64% yield, 98.98% purity) was obtained as a colorless oil. ¹H NMR (400 MHz, CDCl₃) δ ppm 5.61 (s, 1H) 5.42-5.30 (m, 12H) 4.75 (s, 1H) 4.17-4.09 (m, 8H) 3.46 (s, 2H) 2.94-2.85 (m, 2H) 2.79-2.76 (t, J=6.8 Hz, 6H) 2.39-2.35 (m, 2H) 2.33-2.29 (t, J=7.6 Hz, 6H) 2.08-2.03 (m, 12H) 1.96-1.89 (m, 4H) 1.87-1.74 (m, 2H) 1.62-1.59 (m, 8H) 1.49-1.27 (m, 54H) 0.94-0.86 (m, 12H).

### Example 10—Compound 3

### [2-[[2-(1-adamantyl)acetyl]oxymethyl]-2-[[(Z)-octadec-9-enoyl]oxymethyl]-3-[4-(2-pyrrolidin-1ylethylcarbamoyloxy)decanoyloxy]propyl](Z)-octadec-9-enoate

### Step 1:

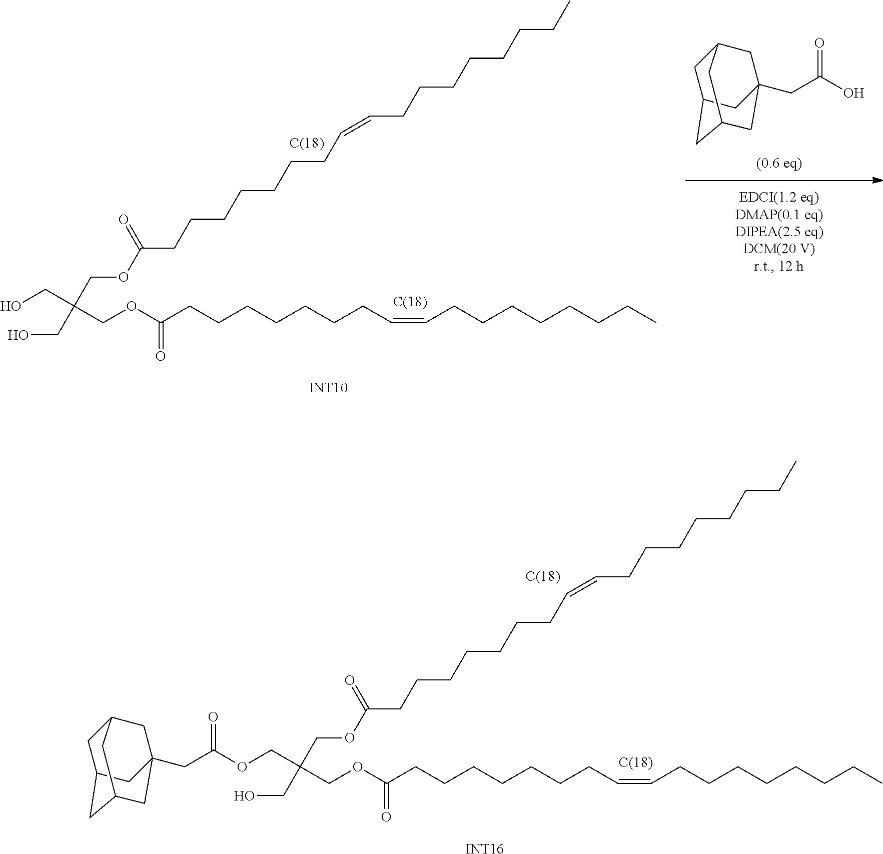

To a mixture of [2,2-bis(hydroxymethyl)-3-[(Z)-octadec-9-enoyl]oxy-propyl](Z)-octadec-9-enoate (3 g, 4.51 mmol, 1 eq), EDCI (1.04 g, 5.41 mmol, 1.2 eq), DIPEA (1.46 g, 11.28 mmol, 1.96 mL, 2.5 eq) and DMAP (55.11 mg, 451.10 mol, 0.1 eq) in DCM (30 mL) was added 2-(1-adamantyl)acetic acid (525.81 mg, 2.71 mmol, 0.6 eq) in DCM (30 mL) slowly. The reaction mixture was stirred at 20° C. for 12 hr under N₂. The reaction mixture was diluted with water (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound [2-[[2-(1-adamantyl)acetyl]oxymethyl]-2-(hydroxymethyl)-3-[(Z)-octadec-9-enoyl]oxy-propyl](Z)-octadec-9-enoate (1.5 g, 1.78 mmol, 39.52% yield) was obtained as a colorless oil. ¹H NMR (400 MHz, CDCl₃) δ ppm 5.40-5.31 (m, 4H) 4.12-4.09 (m, 6H) 3.52-3.50 (d, J=6.8 Hz, 2H) 2.54-2.50 (t, J=7.2 Hz, 1H) 2.35-2.31 (t, J=7.6 Hz, 4H) 2.10 (s, 2H) 2.04-1.98 (m, 9H) 1.73-1.57 (m, 16H) 1.31-1.28 (m, 42H) 0.91-0.87 (t, J=6.8 Hz, 6H).

### Step 2:

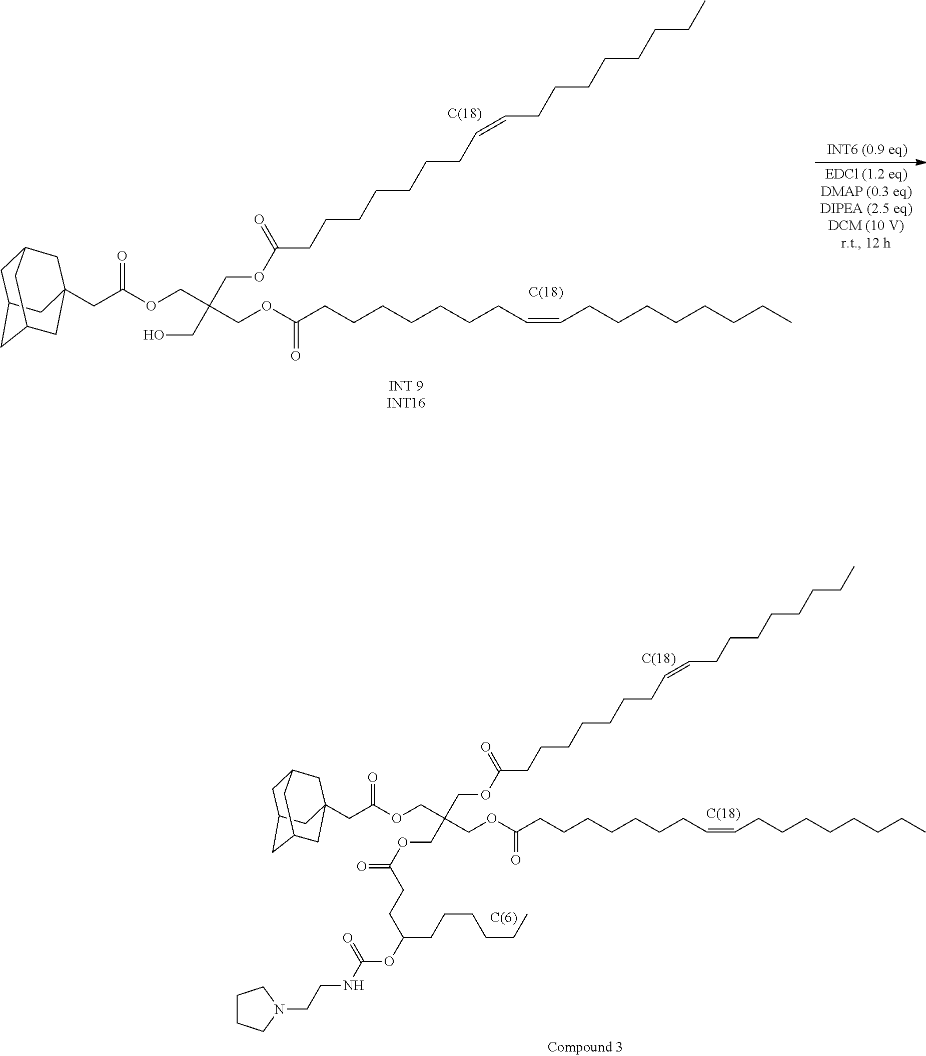 [SMILES: CCCCCCCC/C=C\CCCCCCCC(=O)OCC(CO)(COC(=O)CCCCCCC/C=C\CCCCCCCC)COC(=O)CC12CC3CC(CC(C3)C1)C2 ; "INT 9INT16" ; CCCCCCCC/C=C\CCCCCCCC(=O)OCC(COC(=O)CCCCCCC/C=C\CCCCCCCC)(COC(=O)CCC(CCCCCC)OC(=O)NCCN1CCCC1)COC(=O)CC12CC3CC(CC(C3)C1)C2]

To a mixture of [2-[[2-(1-adamantyl)acetyl]oxymethyl]-2-(hydroxymethyl)-3-[(Z)-octadec-9-enoyl]oxy-propyl](Z)-octadec-9-enoate (1 g, 1.19 mmol, 1 eq) and 4-(2-pyrrolidin-1-ylethylcarbamoyloxy)decanoic acid (351.37 mg, 1.07 mmol, 0.9 eq) in DCM (10 mL) was added EDCI (273.44 mg, 1.43 mmol, 1.2 eq), DMAP (43.56 mg, 356.60 mol, 0.3 eq) and DIPEA (384.06 mg, 2.97 mmol, 517.60 μL, 2.5 eq). The reaction mixture was stirred at 20° C. for 12 hr under N₂ atmosphere. The reaction mixture was diluted with water (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 0/1). Compound [2-[[2-(1-adamantyl)acetyl]oxymethyl]-2-[[(Z)-octadec-9-enoyl]oxymethyl]-3-[4-(2-pyrrolidin-1ylethylcarbamoyloxy)decanoyloxy]propyl](Z)-octadec-9-enoate (105 mg, 91.17 mol, 7.67% yield) was obtained as a colorless oil. ¹H NMR (400 MHz, CDCl₃) δ ppm 5.39-5.31 (m, 4H) 4.75 (s, 1H) 4.16-4.09 (m, 8H) 3.39 (s, 2H) 2.86-2.63 (m, 4H) 2.40-2.35 (m, 2H) 2.33-2.29 (t, J=7.6 Hz, 4H) 2.08 (s, 2H) 2.02-1.97 (m, 10H) 1.89 (s, 4H) 1.82-1.77 (m, 2H) 1.72-1.69 (m, 4H) 1.63-1.58 (m, 14H) 1.49-1.42 (m, 2H) 1.30-1.27 (m, 50H) 0.90-0.87 (m, 9H).

### Example 11—Compound 4

### [2-(4,4-dioctoxybutanoyloxymethyl)-2-[[(Z)-octadec-9-enoyl]oxymethyl]-3-[4-(2-pyrrolidin-1-ylethylcarbamoyloxy)decanoyloxy]propyl](Z)-octadec-9-enoate

### Step 1:

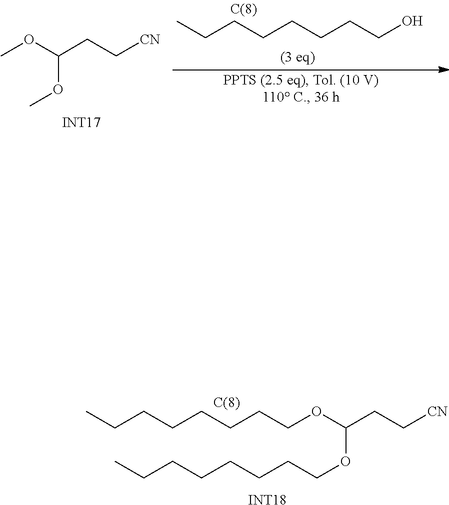

A mixture of 4,4-dimethoxybutanenitrile (30 g, 232.28 mmol, 1 eq), octan-1-ol (90.75 g, 696.83 mmol, 110.13 mL, 3 eq) and PPTS (145.93 g, 580.69 mmol, 2.5 eq) in toluene (300 ml) was degassed and purged with N₂ for 3 times, and then the mixture was stirred at 110° C. for 36 hr under N₂ atmosphere. The reaction mixture was concentrated under reduced pressure to give a residue. The residue was purified by column chromatography (SiO₂, Petroleum ether/Ethyl acetate=1/0 to 50/1). Compound 4,4-dioctoxybutanenitrile (40 g, 122.88 mmol, 52.90% yield) was obtained as a colorless oil.

### Step 2:

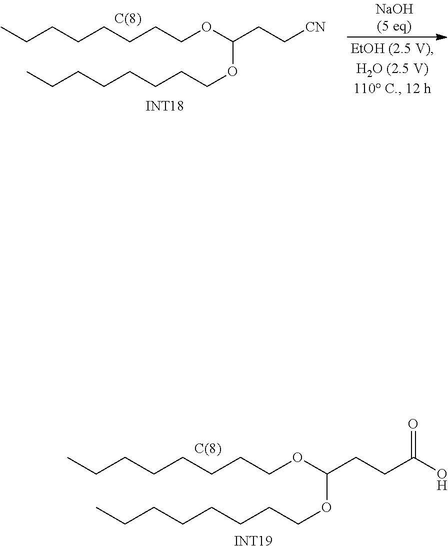

To a solution of 4,4-dioctoxybutanenitrile (40 g, 122.88 mmol, 1 eq) in EtOH (100 mL) and H₂O (100 mL) was added NaOH (24.57 g, 614.39 mmol, 5 eq) slowly. The reaction mixture was stirred at 110° C. for 12 hr under N₂ atmosphere. The reaction mixture was adjusted pH=5 by using 1M HCl and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 0/1). Compound 4,4-dioctoxybutanoic acid (30 g, 87.08 mmol, 70.86% yield) was obtained as a colorless oil. ¹H NMR (400 MHz, CDCl₃) δ ppm 4.54-4.51 (t, J=5.6 Hz, 1H) 3.60-3.56 (m, 2H) 3.45- 3.40 (m, 2H) 2.47-2.44 (t, J=7.6 Hz, 2H) 1.97-1.92 (m, 2H) 1.58-1.55 (m, 4H) 1.32-1.28 (m, 20H) 0.90-0.87 (t, J=6.8 Hz, 6H).

### Step 3:

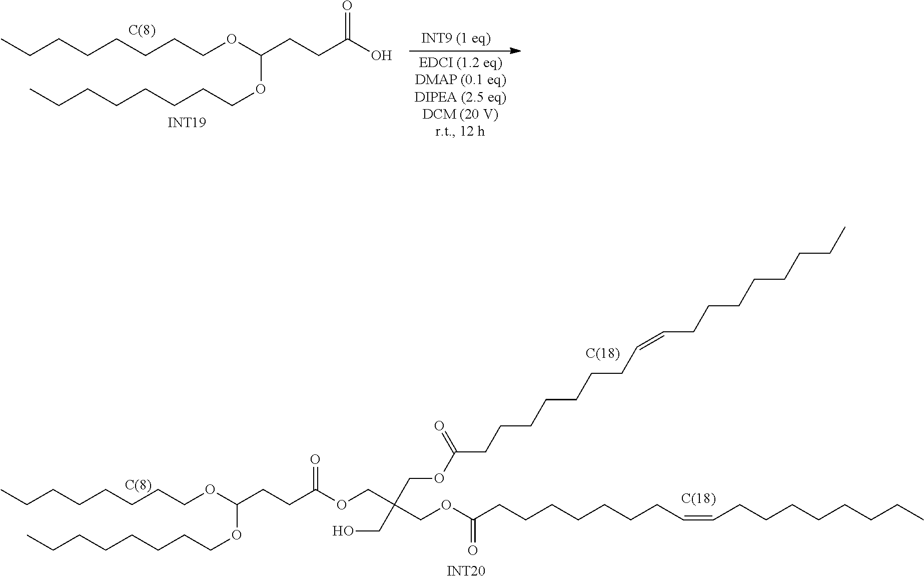

To a solution of [2,2-bis(hydroxymethyl)-3-[(Z)-octadec-9-enoyl]oxy-propyl](Z)-octadec-9-enoate (3 g, 4.51 mmol, 1 eq), EDCI (1.04 g, 5.41 mmol, 1.2 eq), DMAP (55.11 mg, 451.10 mol, 0.1 eq) and DIPEA (1.46 g, 11.28 mmol, 1.96 mL, 2.5 eq) in DCM (30 mL) was added 4,4-dioctoxybutanoic acid (1.55 g, 4.51 mmol, 1 eq) in DCM (30 mL) slowly. The reaction mixture was stirred at 20° C. for 12 hours under N₂ atmosphere. The reaction mixture was diluted with water (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound [2-(4,4-dioctoxybutanoyloxymethyl)-2-(hydroxymethyl)-3-[(Z)-octadec-9-enoyl]oxy-propyl](Z)-octadec-9-enoate (1.5 g, 1.51 mmol, 33.54% yield) was obtained as a colorless oil. ¹H NMR (400 MHz, CDCl₃) δ ppm 5.39-5.30 (m, 4H) 4.50-4.47 (t, J=5.6 Hz, 1H) 4.11 (s, 6H) 3.59-3.50 (m, 4H) 3.43-3.38 (m, 2H) 2.61-2.57 (t, J=6.8 Hz, 1H) 2.44-2.40 (t, J=7.6 Hz, 2H) 2.34-2.31 (t, J=7.6 Hz, 4H) 2.02-1.91 (m, 10H) 1.63-1.54 (m, 8H) 1.31-1.28 (m, 60H) 0.90-0.87 (t, J=6.8 Hz, 12H).

### Step 4:

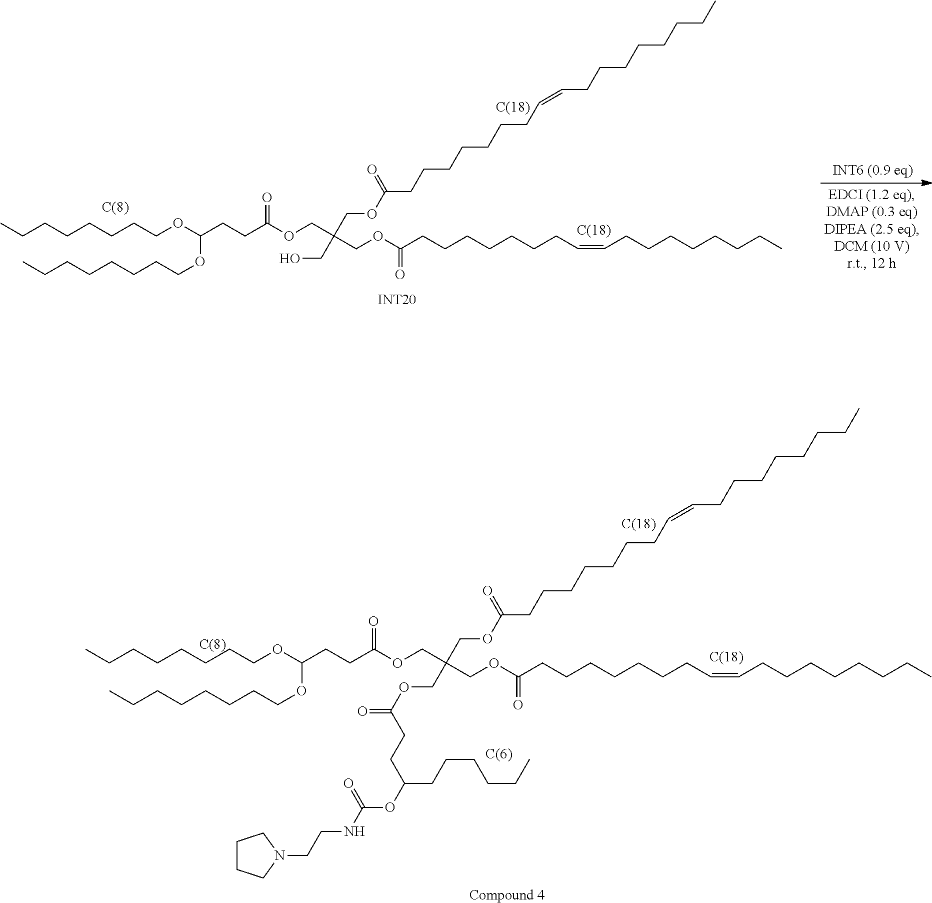

A mixture of [2-(4,4-dioctoxybutanoyloxymethyl)-2-(hydroxymethyl)-3-[(Z)-octadec-9-enoyl]oxy-propyl](Z)-octadec-9-enoate (1.5 g, 1.51 mmol, 1 eq), 4-(2-pyrrolidin-1-ylethylcarbamoyloxy)decanoic acid (447.18 mg, 1.36 mmol, 0.9 eq), EDCI (348.00 mg, 1.82 mmol, 1.2 eq), DMAP (55.44 mg, 453.84 mol, 0.3 eq) and DIPEA (488.79 mg, 3.78 mmol, 658.75 μL, 2.5 eq) in DCM (20 mL) was degassed and purged with N₂ for 3 times, and then the mixture was stirred at 20° C. for 12 hr under N₂ atmosphere. The reaction mixture was diluted with water (50 ml) and extracted with DCM 150 mL (50 ml*3). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 0/1). Compound [2-(4,4-dioctoxybutanoyloxymethyl)-2-[[(Z)-octadec-9-enoyl]oxymethyl]-3-[4-(2-pyrrolidin-1-ylethylcarbamoyloxy)decanoyloxy]propyl](Z)-octadec-9-enoate (110 mg, 84.49 mol, 5.58% yield) was obtained as a colorless oil. ¹H NMR (400 MHz, CDCl₃) δ ppm 5.40-5.31 (m, 4H) 4.76 (s, 1H) 4.49-4.47 (t, J=5.2 Hz, 1H) 4.15-4.11 (m, 8H) 3.59-3.53 (m, 2H) 3.43-3.38 (m, 4H) 2.69 (s, 4H) 2.44-2.26 (m, 8H) 2.02-1.99 (m, 8H) 1.94-1.73 (m, 8H) 1.68-1.05 (m, 84H) 0.90-0.87 (t, J=6.8 Hz, 15H)

### Example 12—Compound 5

### [2-[[2-(1-adamantyl)acetyl]oxymethyl]-2-[[(9Z,12Z)-octadeca-9,12-dienoyl]oxymethyl]-3-[4-(2-pyrrolidin-1-ylethylcarbamoyloxy)decanoyloxy]propyl](9Z,12Z)-octadeca-9,12-dienoate

### Step 1:

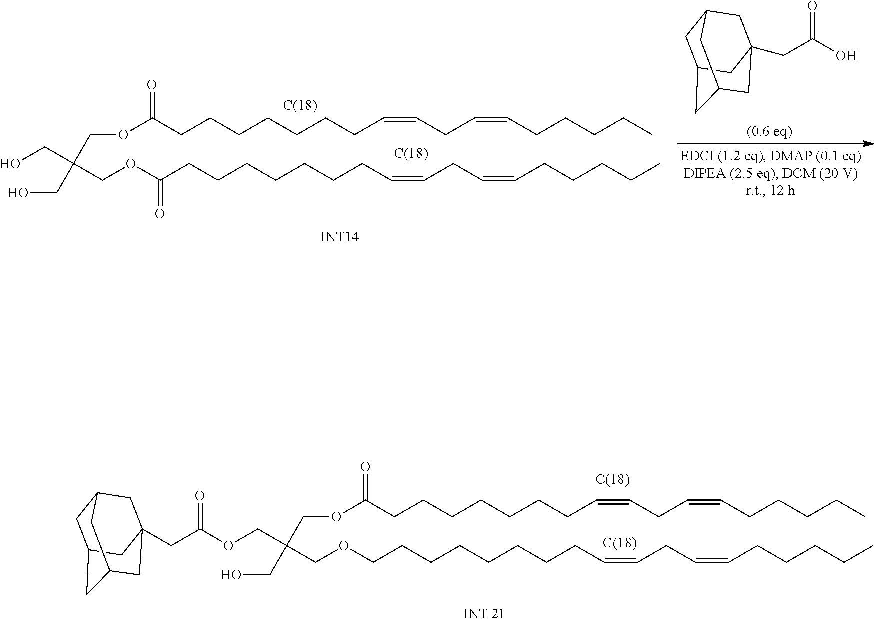

A mixture of [2,2-bis(hydroxymethyl)-3-[(9Z,12Z)-octadeca-9,12-dienoyl]oxy-propyl](9Z,12Z)-octadeca-9,12-dienoate (3 g, 4.54 mmol, 1 eq), 2-(1-adamantyl)acetic acid (529.02 mg, 2.72 mmol, 0.6 eq), DMAP (55.45 mg, 453.86 mol, 0.1 eq), EDCI (1.04 g, 5.45 mmol, 1.2 eq) and DIPEA (1.47 g, 11.35 mmol, 1.98 mL, 2.5 eq) in DCM (30 mL) was degassed and purged with N₂ for 3 times, and then the mixture was stirred at 25° C. for 12 hr under N₂ atmosphere. The reaction mixture was diluted with H₂O (100 mL) and extracted with ethyl acetate 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2-[[2-(1-adamantyl) acetyl]oxymethyl]-2 -(hydroxymethyl)-3-[(9Z,12Z)-octadeca-9,12-dienoyl]oxy-propyl](9Z,12Z)-octadeca-9,12-dienoate (800 mg, 955.50 mol, 21.05% yield) was obtained as a colorless oil. ¹H NMR (400 MHz, CDCl₃) δ ppm 7.77 (d, J=5.4 Hz, 1H), 3.97-3.87 (m, 1H), 3.81-3.68 (m, 4H), 3.48 (s, 3H), 2.87-2.69 (m, 6H), 2.40-2.37 (t, J=6.6 Hz, 2H), 1.99-1.94 (m, 2H), 1.20-1.17 (t, J=7.2 Hz, 6H).

### Step 2:

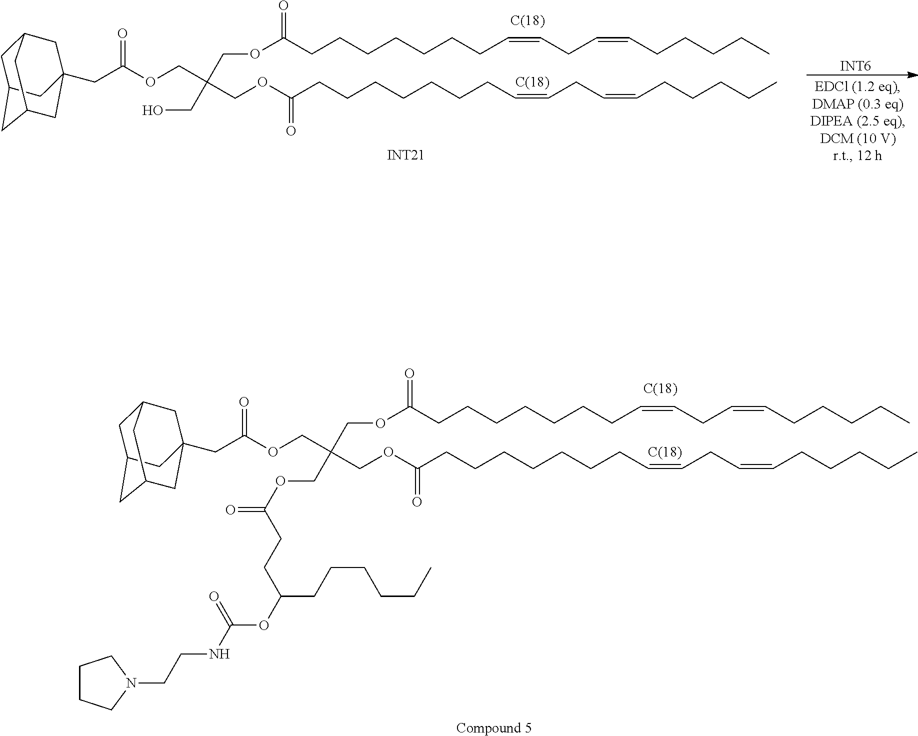

A mixture of [2-[[2-(1-adamantyl)acetyl]oxymethyl]-2-(hydroxymethyl)-3-[(9Z,12Z)-octadeca-9,12-dienoyl]oxy-propyl](9Z,12Z)-octadeca-9,12-dienoate (800 mg, 955.50 mol, 1 eq), 4-(2-pyrrolidin-1-ylethylcarbamoyloxy)decanoic acid (627.66 mg, 1.91 mmol, 2 eq), DMAP (35.02 mg, 286.65 mol, 0.3 eq), DIPEA (308.73 mg, 2.39 mmol, 416.08 L, 2.5 eq) and EDCI (219.81 mg, 1.15 mmol, 1.2 eq) in DCM (8 mL) was degassed and purged with N₂ for 3 times, and then the mixture was stirred at 25° C. for 12 hr under N₂ atmosphere. The reaction mixture was diluted with H₂O (30 mL) and extracted with ethyl acetate 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2-[[2-(1-adamantyl)acetyl]oxymethyl]-2-[[(9Z,12Z)-octadeca-9,12-dienoyl]oxymethyl]-3-[4-(2 -pyrrolidin-1-ylethylcarbamoyloxy)decanoyloxy]propyl](9Z,12Z)-octadeca-9,12-dienoate (140 mg, 121.98 mol, 12.77% yield) was obtained as a colorless oil. ¹H NMR (400 MHz, CDCl₃) δ ppm 5.54-5.24 (m, 8H), 5.18 (d, J=1.0 Hz, 1H), 4.75 (s, 1H), 4.16-4.03 (m, 8H), 3.39-3.21 (m, 2H), 2.79-2.76 (t, J=6.4 Hz, 4H), 2.69-2.44 (m, 6H), 2.40-2.28 (m, 6H), 2.10-2.01 (m, 10H), 2.00 (m, 1H), 1.97 (s, 2H), 1.79 (s, 5H), 1.72 (s, 1H), 1.69 (s, 2H), 1.65-1.54 (m, 14H), 1.42-1.21 (m, 38H), 0.91-0.87 (q, J=6.6 Hz, 9H).

### Example 13—Compound 6

### [2-(4,4-dioctoxybutanoyloxymethyl)-2-[[(9Z,12Z)-octadeca-9,12-dienoyl]oxymethyl]-3-[4-(2-pyrrolidin-1-ylethylcarbamoyloxy)decanoyloxy]propyl](9Z,12Z)-octadeca-9,12-dienoate

### Step 1:

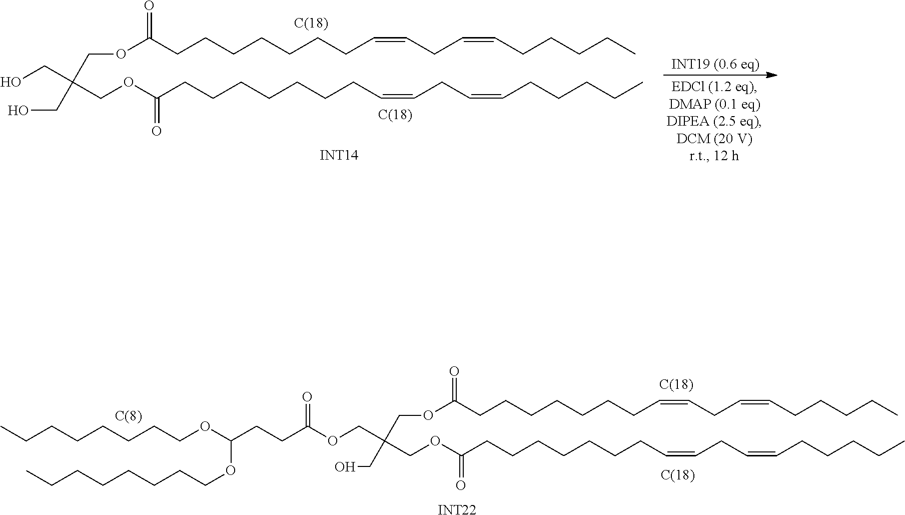

A mixture of [2,2-bis(hydroxymethyl)-3-[(9Z,12Z)-octadeca-9,12-dienoyl]oxy-propyl](9Z,12Z)-octadeca-9,12-dienoate (3 g, 4.54 mmol, 1 eq), 4,4-dioctoxybutanoic acid (938.19 mg, 2.72 mmol, 0.6 eq), EDCI (1.04 g, 5.45 mmol, 1.2 eq), DMAP (55.45 mg, 453.86 mol, 0.1 eq) and DIPEA (1.47 g, 11.35 mmol, 1.98 mL, 2.5 eq) in DCM (60 mL) was degassed and purged with N₂ for 3 times, and then the mixture was stirred at 25° C. for 12 hr under N₂ atmosphere. The reaction mixture was diluted with H₂O (300 mL) and extracted with ethyl acetate 900 mL (300 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2-(4,4-dioctoxybutanoyloxymethyl)-2-(hydroxymethyl)-3-[(9Z,12Z)-octadeca-9,12-dienoyl]oxy-propyl](9Z,12Z)-octadeca-9,12-dienoate (900 mg, 911.38 mol, 20.08% yield) was obtained as a colorless oil. ¹H NMR (400 MHz, CDCl₃) δ ppm 5.41-5.32 (m, 8H), 4.50-4.47 (t, J=5.2 Hz, 1H), 4.12 (s, 6H), 3.58-3.50 (m, 4H), 3.44- 3.38 (m, 2H), 2.80-2.76 (t, J=6.4 Hz, 4H), 2.60-2.56 (t, J=6.8 Hz, 1H), 2.44-2.40 (t, J=7.2 Hz, 2H), 2.35-2.31 (t, J=7.6 Hz, 4H), 2.08-2.03 (q, J=6.8 Hz, J=13.6 Hz, 8H), 1.96-1.91 (q, J=7.6 Hz, J=13.2 Hz, 2H), 1.64-1.55 (m, 8H), 1.40-1.28 (m, 48H), 0.92-0.87 (m, 12H)

### Step 2:

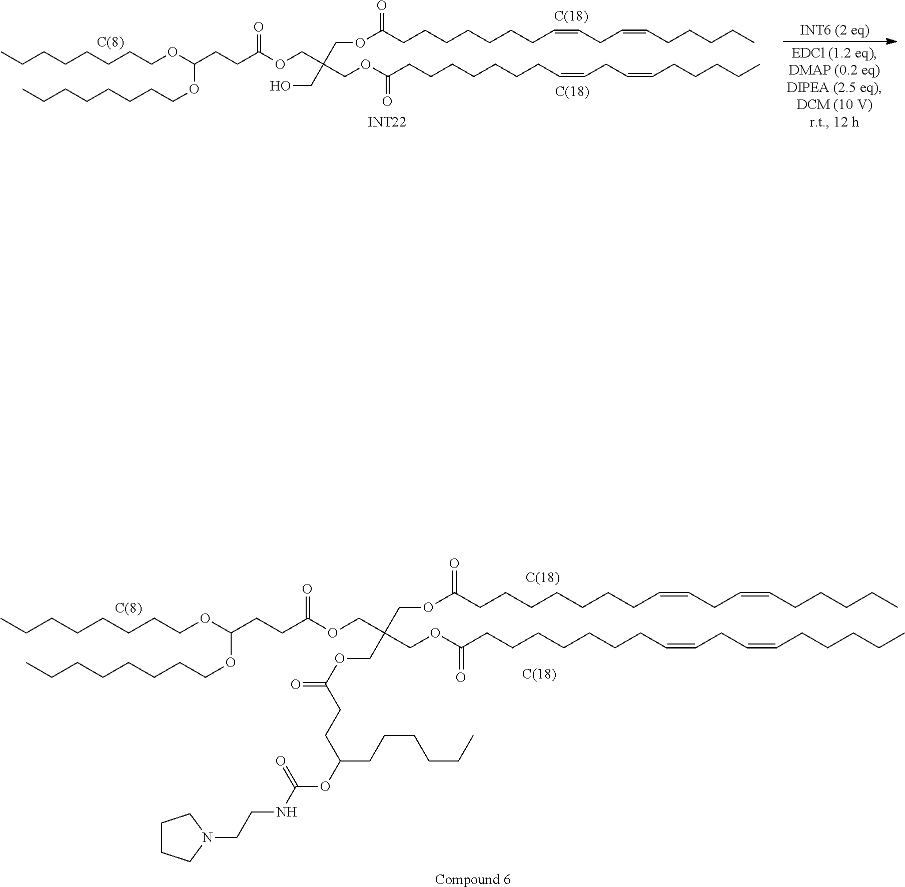

A mixture of [2-(4,4-dioctoxybutanoyloxymethyl)-2-(hydroxymethyl)-3-[(9Z,12Z)-octadeca-9,12-dienoyl]oxy-propyl](9Z,2Z)-octadeca-9,12-dienoate (900 mg, 911.38 mol, 1 eq), 4-(2-pyrrolidin-1-ylethylcarbamoyloxy)decanoic acid (598.68 mg, 1.82 mmol, 2 eq), EDCI (209.66 mg, 1.09 mmol, 1.2 eq), DIPEA (294.47 mg, 2.28 mmol, 396.86 L, 2.5 eq) and DMAP (22.27 mg, 182.28 mol, 0.2 eq) in DCM (9 mL) was degassed and purged with N₂ for 3 times, and then the mixture was stirred at 25° C. for 12 hr under N₂ atmosphere. The reaction mixture was diluted with H₂O (30 mL) and extracted with ethyl acetate 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound [2-(4,4-dioctoxybutanoyloxymethyl)-2-[[(9Z,12Z)-octadeca-9,12-dienoyl]oxymethyl]-3-[4-(2-pyrrolidin-1-ylethylcarbamoyloxy)decanoyloxy]propyl](9Z, 12Z)-octadeca-9,12-dienoate (140 mg, 103.69 mol, 11.38% yield, 96.13% purity) was obtained as a colorless oil. ¹H NMR (400 MHz, CHLOROFORM-d) δ ppm 5.42-5.30 (m, 8H), 5.18 (s, 1H), 4.75 (s, 1H), 4.49-4.47 (t, J=5.2 Hz, 1H), 4.12-4.11 (d, J=3.2 Hz, 8H), 3.59-3.53 (m, 2H), 3.43-3.38 (m, 2H), 3.302-3.289 (d, J=5.2 Hz, 2H), 2.79-2.76 (t, J=6.8 Hz, 4H), 2.62-2.59 (t, J=5.6 Hz, 2H), 2.53 (s, 4H), 2.42-2.35 (m, 4H), 2.32-2.29 (t, J=7.6 Hz, 4H), 2.08-2.03 (q, J=6.4 Hz, J=13.2 Hz, 8H), 1.94-1.89 (q, J=6.4 Hz, J=13.2 Hz, 3H), 1.78 (s, 3H), 1.62-1.53 (m, 10H), 1.40-1.28 (m, 58H), 0.91-0.87 (m, 15H).

### Example 14—Compound 7

### O7-[2,2-bis[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate

### Step 1:

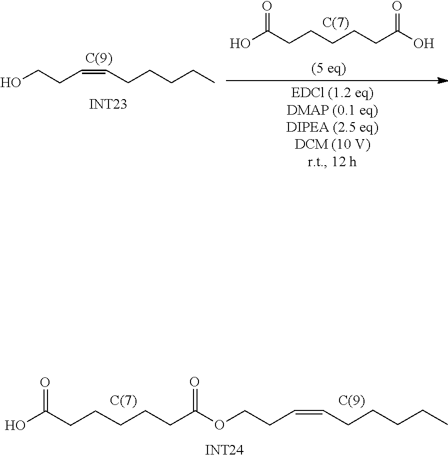

A mixture of (Z)-non-3-en-1-ol (50 g, 351.52 mmol, 1 eq), heptanedioic acid (281.51 g, 1.76 mol, 5 eq), EDCI (80.87 g, 421.83 mmol, 1.2 eq), DMAP (4.29 g, 35.15 mmol, 0.1 eq) and DIPEA (113.58 g, 878.81 mmol, 153.07 mL, 2.5 eq) in DCM (500 mL) was degassed and purged with N₂ for 3 times, and then the mixture was stirred at 20° C. for 12 hr under N₂ atmosphere. The reaction mixture was diluted with water (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound 7-[(Z)-non-3-enoxy]-7-oxo-heptanoic acid (140 g, 492.28 mmol, 35.01% yield) was obtained as a colorless oil. ¹H NMR (400 MHz, CDCl₃) δ ppm 11.63-10.83 (m, 1H), 5.52-5.49 (m, 1H), 5.35-5.30 (m, 1H), 4.09-4.05 (t, J=6.8 Hz, 2H), 2.04-2.29 (m, 6H), 2.07-2.01 (m, 2H), 1.70-1.62 (m, 4H), 1.43-1.28 (m, 8H), 0.91-0.87 (t, J=6.8 Hz, 3H)

### Step 2:

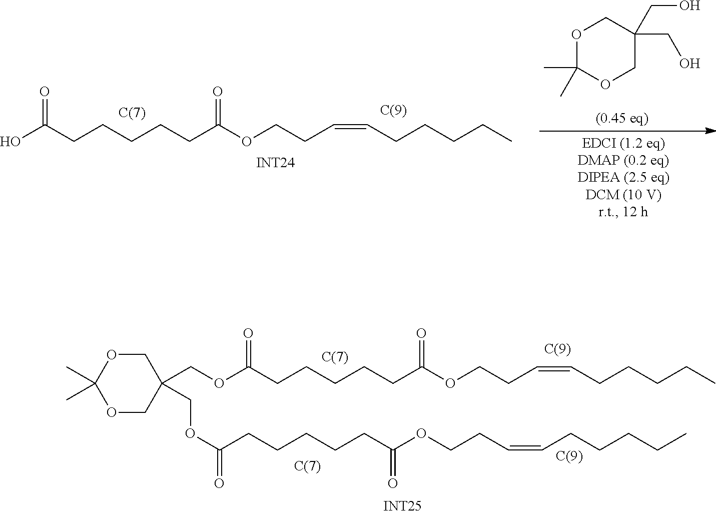

A mixture of 7-[(Z)-non-3-enoxy]-7-oxo-heptanoic acid (28.69 g, 100.89 mmol, 1 eq), [5-(hydroxymethyl)-2,2-dimethyl-1,3-dioxan-5-yl]methanol (8 g, 45.40 mmol, 0.45 eq), EDCI (23.21 g, 121.07 mmol, 1.2 eq), DIPEA (32.60 g, 252.22 mmol, 43.93 mL, 2.5 eq) and DMAP (2.47 g, 20.18 mmol, 0.2 eq) in DCM (300 mL) was degassed and purged with N₂ for 3 times, and then the mixture was stirred at 20° C. for 12 hr under N₂ atmosphere. The reaction mixture was diluted with water (200 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound O7-[[2,2-dimethyl-5-[[7-[(Z)-non-3-enoxy]-7-oxo-heptanoyl]oxymethyl]-1,3-dioxan-5-yl]methyl]O1-[(Z)-non-3-enyl]heptanedioate (70 g, 98.74 mmol, 65.24% yield) was obtained as a colorless oil. ¹H NMR (400 MHz, CDCl₃) δ ppm 5.54-5.49 (m, 2H), 5.35-5.32 (m, 2H), 4.10 (s, 4H), 4.08-4.05 (t, J=6.8 Hz, 4H), 3.75 (s, 4H), 2.40-2.28 (m, 12H), 2.06-2.01 (q, J=6.8 Hz, J=13.6 Hz, 4H), 1.66-1.62 (m, 8H), 1.42 (s, 6H), 1.37-1.26 (m, 16H), 0.91-0.87 (t, J=6.4 Hz, 6H)

### Step 3:

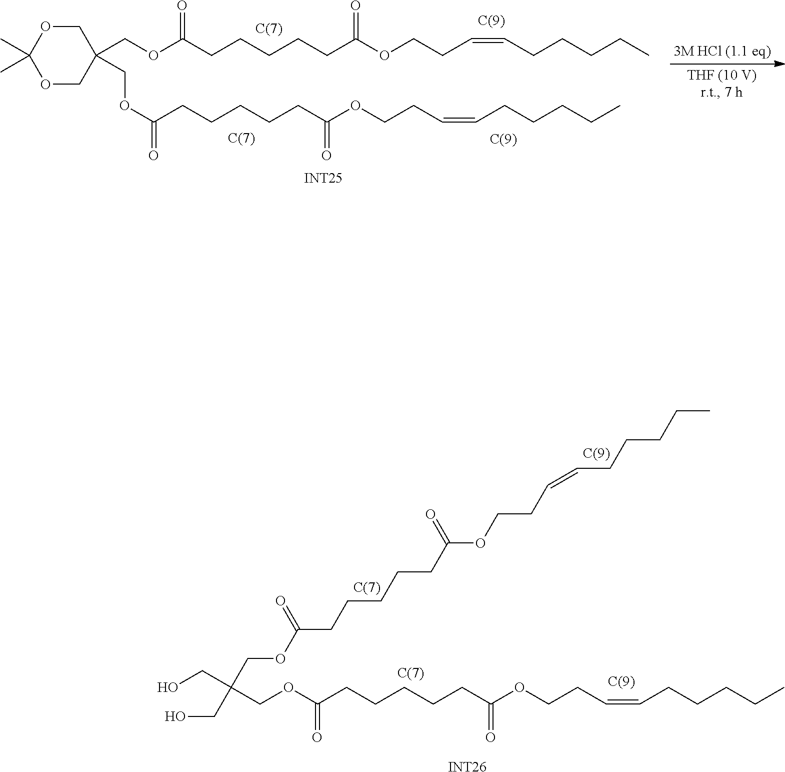

A solution of O7-[[2,2-dimethyl-5-[[7-[(Z)-non-3-enoxy]-7-oxo-heptanoyl]oxymethyl]-1,3-dioxan-5-yl]methyl]O1-[(Z)-non-3-enyl]heptanedioate (30 g, 42.32 mmol, 1 eq) in THF (300 mL) was added HCl (3 M, 15.52 mL, 1.1 eq) slowly and then the mixture was stirred at 20° C. for 12 hr under N₂ atmosphere. The reaction mixture was added sat.Na₂CO₃ (300 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=20/1 to 1/1). Compound O7-[2,2-bis(hydroxymethyl)-3-[7-[(Z)-non-3-enoxy]-7-oxo-heptanoyl]oxy-propyl]O1-[(Z)-non-3-enyl]heptanedioate (20 g, 29.90 mmol, 35.33% yield) was obtained as a colorless oil. ¹H NMR (400 MHz, CDCl₃) δ ppm 5.52-5.47 (m, 2H), 5.36-5.30 (m, 2H), 4.14 (s, 4H), 4.08-4.04 (t, J=7.2 Hz), 3.58 (s, 4H), 2.84 (s, 2H), 2.40-2.29 (m, 12H), 2.06-2.00 (q, J=6.4 Hz, J=14 Hz, 4H), 1.69-1.60 (m, 8H), 1.38-1.28 (m, 16H), 0.91-0.87 (t, J=6.8 Hz, 6H)

### Step 4:

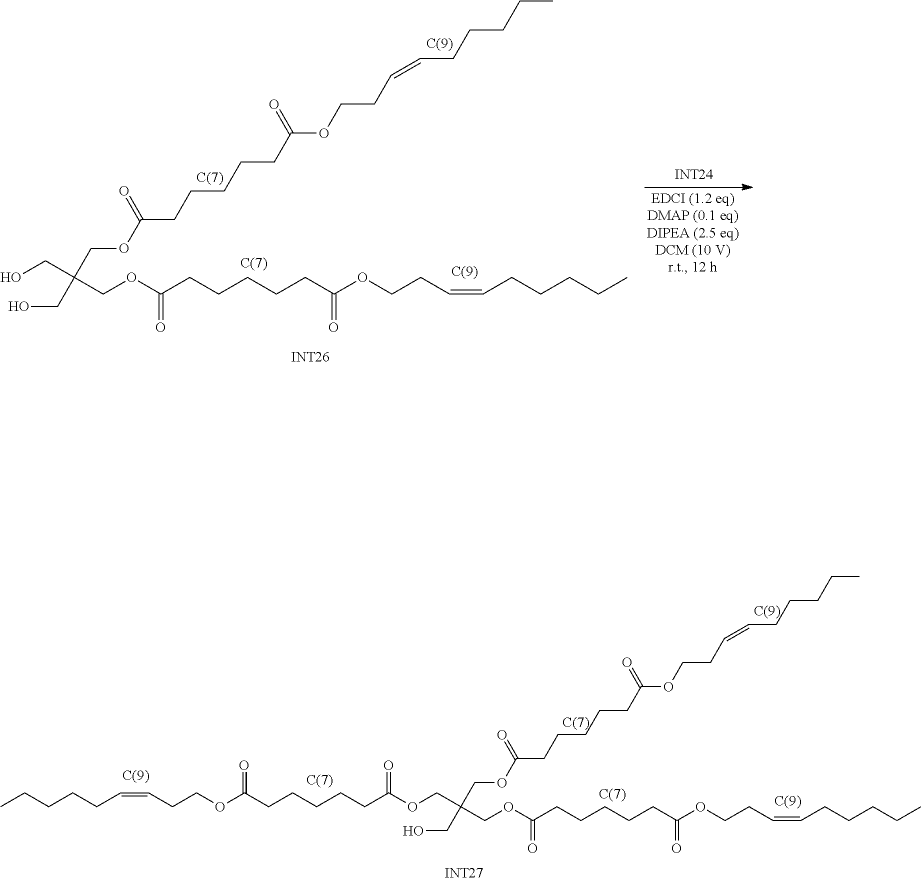

To a mixture of O7-[2,2-bis(hydroxymethyl)-3-[7-[(Z)-non-3-enoxy]-7-oxo-heptanoyl]oxy-propyl]O1-[(Z)-non-3-enyl]heptanedioate (20 g, 29.90 mmol, 1 eq), EDCI (6.88 g, 35.88 mmol, 1.2 eq), DIPEA (9.66 g, 74.75 mmol, 13.02 mL, 2.5 eq), DMAP (365.28 mg, 2.99 mmol, 0.1 eq) in DCM (150 mL) was added 7-[(Z)-non-3-enoxy]-7-oxo-heptanoic acid (5.10 g, 17.94 mmol, 0.6 eq) in DCM (50 mL). The mixture was stirred at 20° C. for 12 hr under N₂ atmosphere. The reaction mixture was diluted with water (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 10/1). Compound O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (8 g, 8.55 mmol, 28.61% yield) was obtained as a colorless oil. ¹H NMR (400 MHz, CDCl₃) δ ppm 5.54-5.49 (m, 3H), 5.35-5.30 (m, 3H), 4.11 (s, 6H), 4.08-4.05 (t, J=7.2 Hz, 6H), 3.52-3.50 (d, J=6 Hz, 2H), 2.67-2.64 (t, J=13.6 Hz, 1H), 2.40-2.29 (m, 18H), 2.06-2.01 (q, J=6.4 Hz, 13.6 Hz, 6H), 1.68-1.60 (m, 12H), 1.39-1.27 (m, 24H), 0.91-0.87 (t, J=6.8 Hz, 9H)

### Step 5:

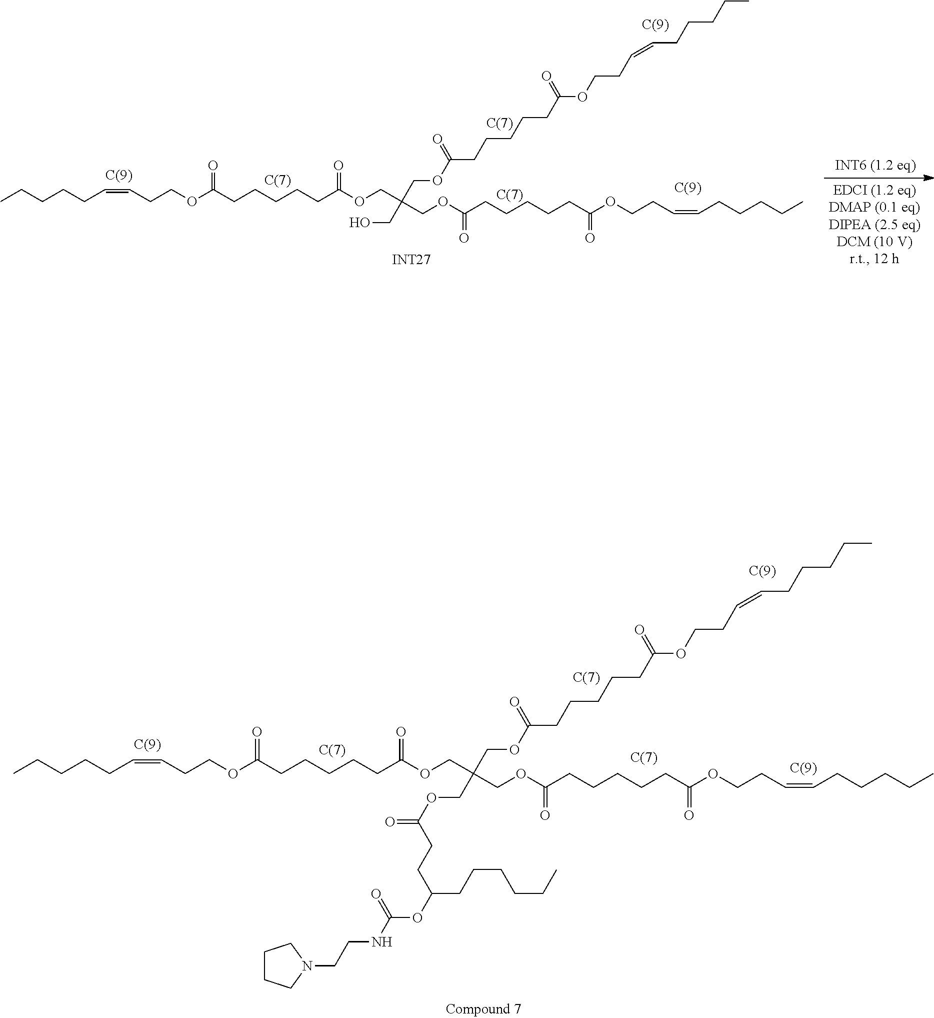

A mixture of O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (8 g, 8.55 mmol, 1 eq), 4-(2-pyrrolidin-1-ylethylcarbamoyloxy)decanoic acid (3.37 g, 10.26 mmol, 1.2 eq), EDCI (1.97 g, 10.26 mmol, 1.2 eq), DIPEA (2.76 g, 21.38 mmol, 3.72 mL, 2.5 eq) and DMAP (104.50 mg, 855.37 mol, 0.1 eq) in DCM (80 mL) was degassed and purged with N₂ for 3 times, and then the mixture was stirred at 20° C. for 12 hr under N₂ atmosphere. The reaction mixture was diluted with water (200 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 10/1). Compound O7-[2,2-bis[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate (3.4 g, 2.73 mmol, 31.91% yield) was obtained as a colorless oil. ¹H NMR (400 MHz, CDCl₃) δ ppm 5.54-5.47 (m, 3H), 5.35-5.30 (m, 3H), 5.18 (s, 1H), 4.75 (s, 1H), 4.10 (s, 8H), 4.08-4.04 (t, J=7.2 Hz, 6H), 3.29-3.28 (d, J=5.2 Hz, 2H), 2.61-2.58 (t, J=5.6 Hz, 2H), 2.52 (s, 4H), 2.40-2.28 (m, 22H), 2.06-2.01 (q, J=6.4 Hz, J=13.6 Hz, 6H), 1.90-1.87 (m, 1H), 1.77 (s, 3H), 1.67-1.59 (m, 14H), 1.37-1.27 (m, 32H), 0.91-0.86 (m, 12H)

### Example 15—Compound 8

### O7-[2-[[2-(1-adamantyl)acetyl]oxymethyl]-2-[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate

### Step 1:

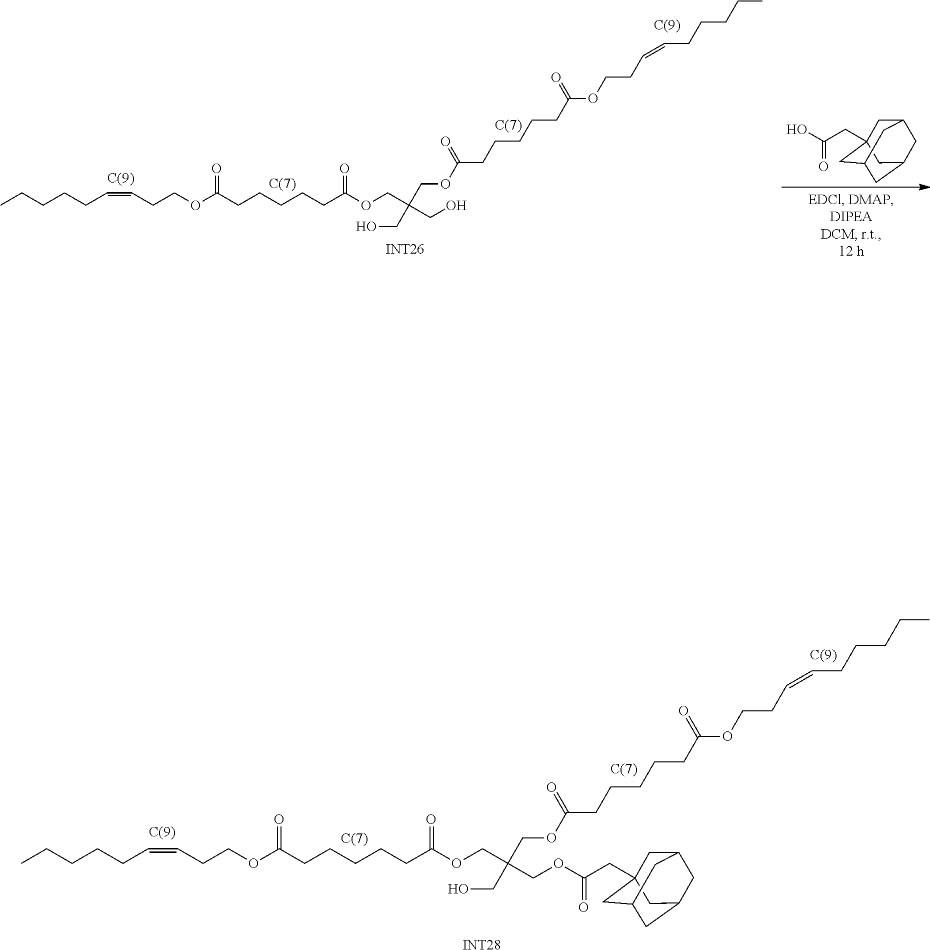

To a solution of O7-[2,2-bis(hydroxymethyl)-3-[7-[(Z)-non-3-enoxy]-7-oxo-heptanoyl]oxy-propyl]O1-[(Z)-non-3-enyl]heptanedioate (1.5 g, 2.24 mmol, 1 eq) in DCM (15 mL) was added EDCI (515.87 mg, 2.69 mmol, 1.2 eq), DIPEA (724.57 mg, 5.61 mmol, 976.51 μL, 2.5 eq) and DMAP (54.79 mg, 448.50 μmol, 0.2 eq). Then a solution of 2-(1-adamantyl)acetic acid (392.08 mg, 2.02 mmol, 0.9 eq) in DCM (15 mL) was added to the above reaction mixture dropwise. The mixture was stirred at 20° C. for 12 hr under N₂. The reaction mixture was diluted with H₂O (30 mL) and extracted with ethyl acetate 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 5/1). Compound O7-[2-[[2-(1-adamantyl)acetyl]oxymethyl]-2-(hydroxymethyl)-3-[7-[(Z)-non-3-enoxy]-7-oxoheptanoyl]oxy-propyl]O1-[(Z)-non-3-enyl]heptanedioate (0.7 g, 828.26 mol, 36.93% yield) was obtained as a colorless oil. ¹H NMR (400 MHz, CDCl₃) δ ppm 5.54-5.48 (m, 2H), 5.37-5.31 (m, 2H), 4.12 (s, 4H), 4.09-4.05 (t, J=6.4 Hz, 6H), 3.53-3.52 (d, J=4 Hz, 2H), 2.61 (s, 1H), 2.40-2.29 (m, 12H), 2.10 (s, 2H), 2.07-2.02 (q, J=6.8 Hz, J=14 Hz, 4H), 1.98 (s, 3H), 1.73-1.60 (m, 20H), 1.40-1.27 (m, 16H), 0.91-0.88 (t, J=6.4 Hz, 6H)

### Step 2:

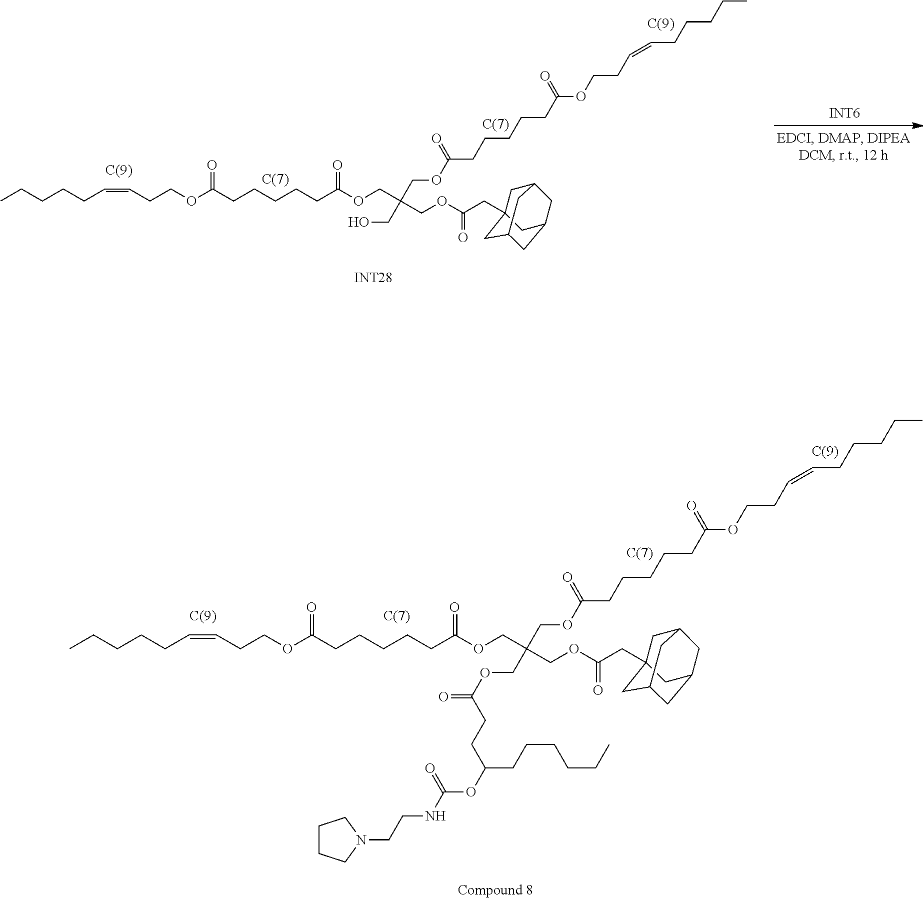

To a solution of 4-(2-pyrrolidin-1-ylethylcarbamoyloxy)decanoic acid (272.04 mg, 828.26 mol, 1 eq) in DCM (7 mL) was added EDCI (190.53 mg, 993.91 mol, 1.2 eq), DIPEA (267.62 mg, 2.07 mmol, 360.67 μL, 2.5 eq) and DMAP (20.24 mg, 165.65 mol, 0.2 eq), then O7-[2-[[2-(1-adamantyl)acetyl]oxymethyl]-2-(hydroxymethyl)-3-[7-[(Z)-non-3-enoxy]-7-oxo-heptanoyl]oxy-propyl]O1-[(Z)-non-3-enyl]heptanedioate (0.7 g, 828.26 mol, 1 eq) was added to the above reaction mixture. The mixture was stirred at 20° C. for 12 hr under N₂. The reaction mixture was diluted with H₂O (30 mL) and extracted with ethyl acetate 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 1/1). Compound O7-[2-[[2-(1-adamantyl)acetyl]oxymethyl]-2-[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate (0.15 g, 129.80 μmol, 15.67% yield) was obtained as a colorless oil. ¹H NMR (400 MHz, CDCl₃) δ ppm 5.54-5.48 (m, 2H), 5.37-5.31 (m, 2H), 5.28- 5.23 (m, 1H), 4.75 (s, 1H), 4.11 (s, 6H), 4.08-4.05 (t, J=6.8 Hz, 6H), 3.309 (s, 2H), 2.63-2.56 (d, J=26.8 Hz, 6H), 2.40-2.29 (m, 14H), 2.08-2.02 (m, 6H), 1.97 (s, 3H), 1.90-1.86 (m, 1H), 1.80 (s, 5H), 1.72-1.58 (m, 22H), 1.39-1.27 (m, 24H), 0.91-0.86 (m, 9H).

### Example 16—Compound 9

### O7-[2-(4,4-dioctoxybutanoyloxymethyl)-2-[[7-[(Z)-non-3-enoxy]-7-oxo-heptanoyl]oxymethyl]-3-[4-(2-pyrrolidin-1ylethylcarbamoyloxy)decanoyloxy]propyl]O1-[(Z)-non-3-enyl]heptanedioate

### Step 1:

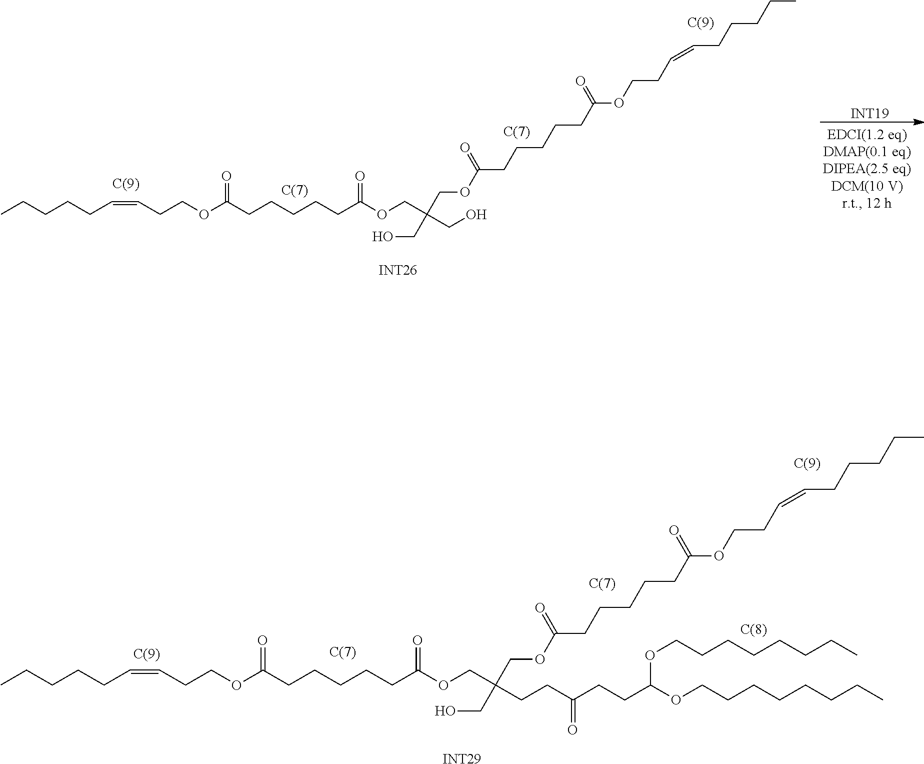

A mixture of O7-[2,2-bis(hydroxymethyl)-3-[7-[(Z)-non-3-enoxy]-7-oxo-heptanoyl]oxy-propyl]O1-[(Z)-non-3-enyl]heptanedioate (3.2 g, 4.78 mmol, 1 eq), 4,4-dioctoxybutanoic acid (824.11 mg, 2.39 mmol, 0.5 eq), DMAP (58.45 mg, 478.40 mol, 0.1 eq), DIPEA (1.55 g, 11.96 mmol, 2.08 mL, 2.5 eq) and EDCI (1.10 g, 5.74 mmol, 1.2 eq) in DCM (32 mL) was degassed and purged with N₂ for 3 times, and then the mixture was stirred at 25° C. for 12 hr under N₂ atmosphere. The reaction mixture was diluted with H₂O (50 mL) and extracted with ethyl acetate 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound O7-[2-(4,4-dioctoxybutanoyloxymethyl)-2-(hydroxymethyl)-3-[7-[(Z)-non-3-enoxy]-7-oxo-heptanoyl]oxy-propyl]O1-[(Z)-non-3-enyl]heptanedioate (1.4 g, 1.41 mmol, 29.40% yield) was obtained as a colorless oil. ¹H NMR (400 MHz, CDCl₃) δ ppm 5.54-5.50 (m, 2H), 5.35-5.33 (m, 2H), 4.50-4.47 (t, J=5.2 Hz, 1H), 4.11 (s, 6H), 4.09-4.05 (t, J=7.2 Hz, 4H), 3.59-3.51 (m, 4H), 3.43-3.38 (m, 2H), 2.67-2.64 (t, J=6.8 Hz, 1H), 2.42-2.29 (m, 14H), 2.07-2.02 (q, J=6.8 Hz, J=14.0 Hz, 4H), 1.96-1.91 (q, J=7.6 Hz, J=13.2 Hz, 2H), 1.68-1.54 (m, 12H), 1.40-1.28 (m, 36H), 0.91-0.87 (m, 12H)

### Step 2:

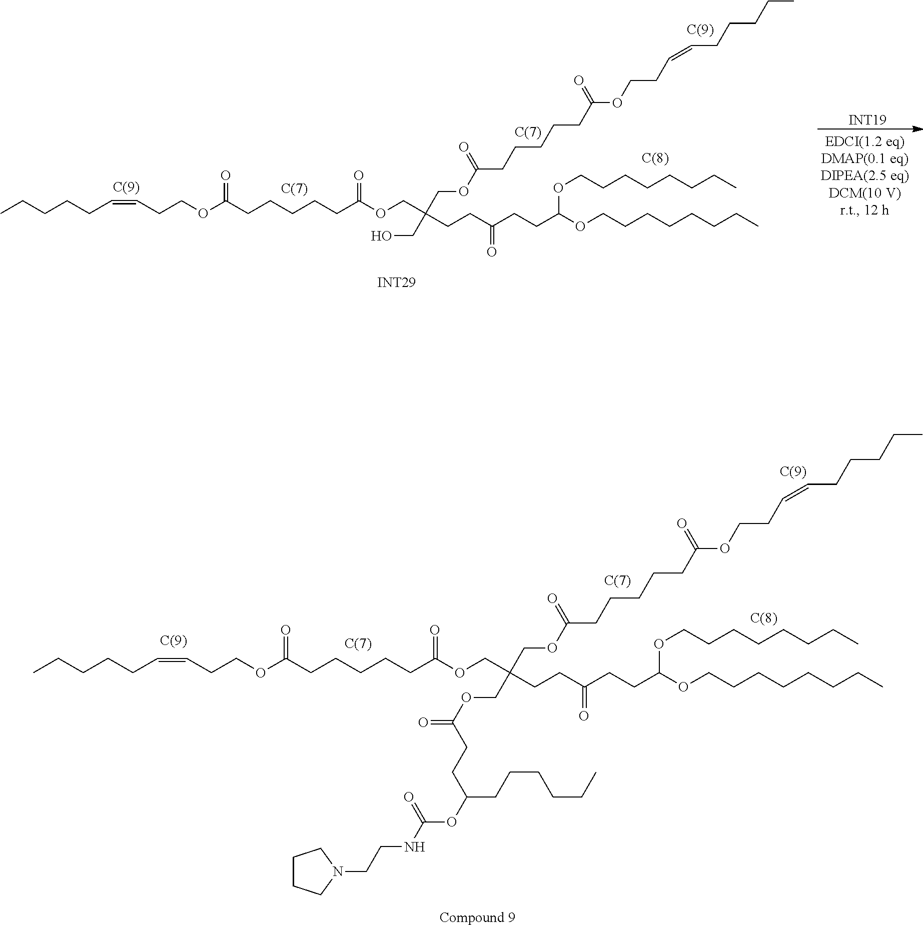

A mixture of O7-[2-(4,4-dioctoxybutanoyloxymethyl)-2-(hydroxymethyl)-3-[7-[(Z)-non-3-enoxy]-7-oxo-heptanoyl]oxy-propyl]O1-[(Z)-non-3-enyl]heptanedioate (1.4 g, 1.41 mmol, 1 eq), 4-(2-pyrrolidin-1-ylethylcarbamoyloxy)decanoic acid (461.95 mg, 1.41 mmol, 1 eq), DMAP (17.18 mg, 140.65 mol, 0.1 eq), EDCI (323.55 mg, 1.69 mmol, 1.2 eq) and DIPEA (454.44 mg, 3.52 mmol, 612.45 μL, 2.5 eq) in DCM (14 mL) was degassed and purged with N₂ for 3 times, and then the mixture was stirred at 25° C. for 12 hr under N₂ atmosphere. The reaction mixture was diluted with H₂O (50 mL) and extracted with ethyl acetate 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound O7-[2-(4,4-dioctoxybutanoyloxymethyl)-2-[[7-[(Z)-non-3-enoxy]-7-oxo-heptanoyl]oxymethyl]-3-[4-(2-pyrrolidin-1ylethylcarbamoyloxy)decanoyloxy]propyl]O1-[(Z)-non-3-enyl]heptanedioate (150 mg, 114.87 mol, 8.17% yield) was obtained as a colorless oil. ¹H NMR (400 MHz, CDCl₃) δ ppm 5.52-5.49 (m, 2H), 5.35-5.32 (m, 2H), 5.19 (s, 1H), 4.74 (s, 1H), 4.49-4.46 (t, J==5.6 Hz, 1H), 4.11-4.10 (d, J=2 Hz, 8H), 4.08-4.04 (t, J=6.8 Hz, 4H), 3.58-3.53 (m, 2H), 3.43-3.37 (m, 2H), 3.30-3.28 (d, J=5.6 Hz, 2H), 2.61-2.58 (t, J=5.6 Hz, 2H), 2.53 (s, 4H), 2.41-2.28 (m, 16H), 2.06-2.01 (q, J=6.8 Hz, J=13.6 Hz, 4H), 1.93-1.88 (q, J=7.6 Hz, J=13.2 Hz, 3H), 1.78 (s, 5H), 1.67-1.52 (m, 14H), 1.37-1.28 (m, 44H), 0.91-0.86 (m, 15H)

### Example 17—Compound 10

### O8-[2,2-bis[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate

### Step 1:

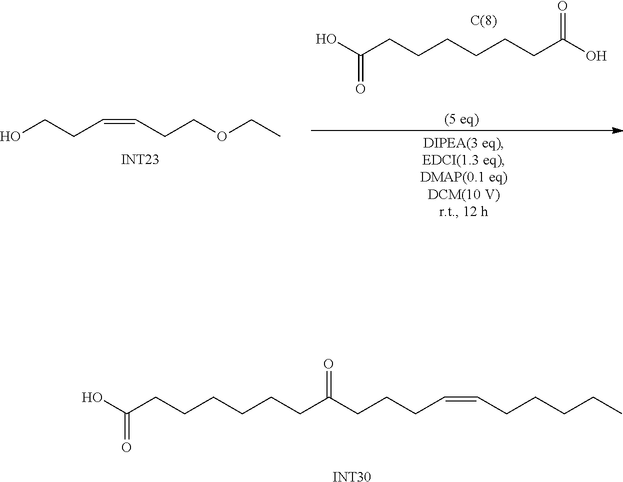

A mixture of (Z)-non-3-en-1-ol (40 g, 281.22 mmol, 1 eq), octanedioic acid (244.93 g, 1.41 mol, 5 eq), EDCI (70.08 g, 365.58 mmol, 1.3 eq), DIPEA (109.04 g, 843.66 mmol, 146.95 mL, 3 eq) and DMAP (3.44 g, 28.12 mmol, 0.1 eq) in DCM (400 mL) was degassed and purged with N₂ for 3 times, and then the mixture was stirred at 20° C. for 12 hr under N₂ atmosphere. The reaction mixture was diluted with water (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound 8-[(Z)-non-3-enoxy]-8-oxo-octanoic acid (140 g, 469.14 mmol, 33.36% yield) was obtained as colourless oil. ¹H NMR (400 MHz, CDCl₃) δ ppm 11.33 (s, 1H), 5.54-5.48 (m, 1H), 5.37-5.31 (m, 1H), 4.09-4.05 (t, J=6.8 Hz, 2H), 2.40-2.28 (m, 6H), 2.07-2.01 (q, J=6.4 Hz, J=14 Hz, 2H), 1.68-1.60 (m, 4H), 1.39-1.27 (m, 10H), 0.91-0.88 (t, J=6.8 Hz, 3H)

### Step 2:

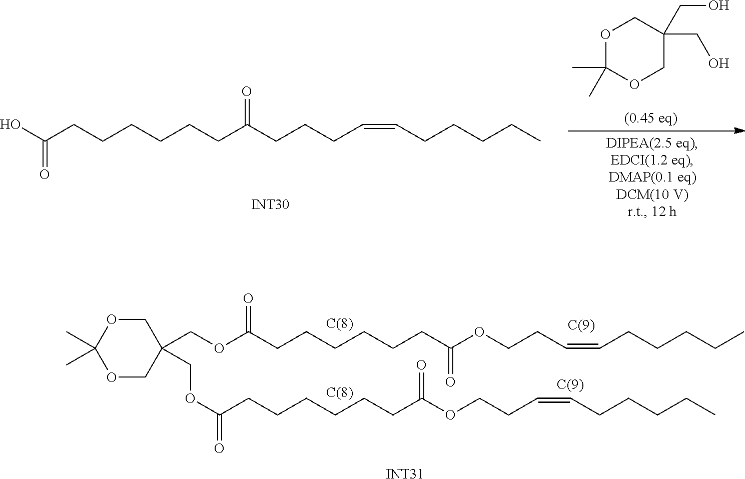

A mixture of 8-[(Z)-non-3-enoxy]-8-oxo-octanoic acid (30.11 g, 100.89 mmol, 1 eq), [5-(hydroxymethyl)-2,2-dimethyl-1,3-dioxan-5-yl]methanol (8 g, 45.40 mmol, 0.45 eq), EDCI (23.21 g, 121.07 mmol, 1.2 eq), DIPEA (32.60 g, 252.22 mmol, 43.93 mL, 2.5 eq) and DMAP (1.23 g, 10.09 mmol, 0.1 eq) in DCM (300 mL) was degassed and purged with N₂ for 3 times, and then the mixture was stirred at 20° C. for 12 hr under N₂ atmosphere. The reaction mixture was diluted with water (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 10/1). Compound O8-[[2,2-dimethyl-5-[[8-[(Z)-non-3-enoxy]-8-oxo-octanoyl]oxymethyl]-1,3-dioxan-5-yl]methyl]O1-[(Z)-non-3-enyl]octanedioate (70 g, 94.98 mmol, 62.76% yield) was obtained as colorless oil. ¹H NMR (400 MHz, CDCl₃) δ ppm 5.54-5.47 (m, 2H), 5.37-5.30 (m, 2H), 4.11 (s, 4H), 4.08-4.05 (t, J=6.8 Hz, 4H), 3.74 (s, 4H), 2.40-2.27 (m, 12H), 2.07-2.01 (q, J=7.2 Hz, J=14 Hz, 4H), 1.64-1.59 (m, 8H), 1.42 (s, 6H), 1.37-1.27 (m, 20H), 0.91-0.88 (t, J=6.8 Hz, 6H).

### Step 3:

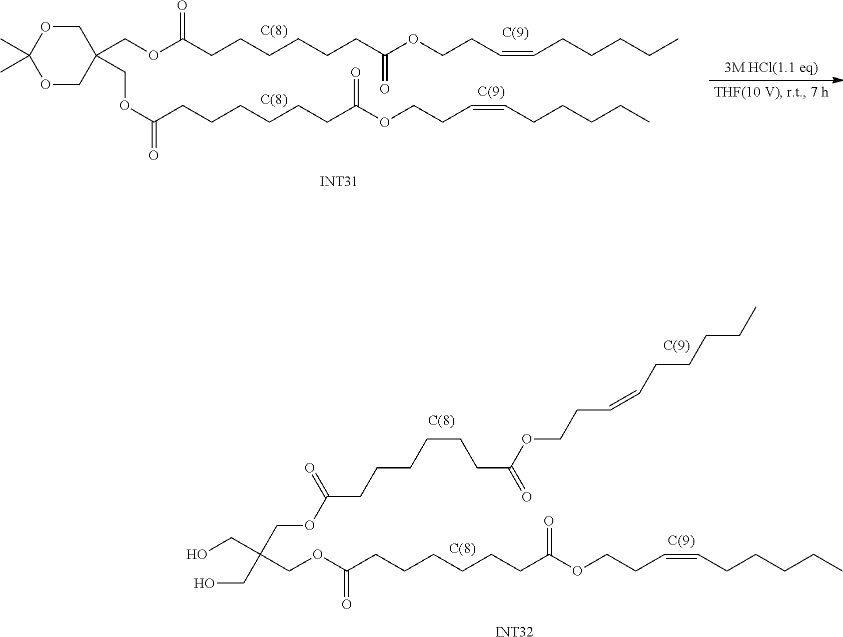

A solution of O8-[[2,2-dimethyl-5-[[8-[(Z)-non-3-enoxy]-8-oxo-octanoyl]oxymethyl]-1,3-dioxan-5-yl]methyl]O1-[(Z)-non-3-enyl]octanedioate (70 g, 94.98 mmol, 1 eq) and HCl (3 M, 34.83 mL, 1.1 eq) in THF (700 mL) was degassed and purged with N₂ for 3 times, and then the mixture was stirred at 20° C. for 7 hr under N₂ atmosphere. The reaction mixture was added sat.Na₂CO₃ 500 mL at 20° C. slowly. Then it was extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=20/1 to 1/1). Compound O8-[2,2-bis(hydroxymethyl)-3-[8-[(Z)-non-3-enoxy]-8-oxo-octanoyl]oxy-propyl]O1-[(Z)-non-3-enyl]octanedioate (38 g, 54.52 mmol, 57.41% yield) was obtained as a colorless oil. ¹H NMR (400 MHz, CDCl₃) δ ppm 5.53-5.47 (m, 2H), 5.37-5.30 (m, 2H), 4.14 (s, 1H), 4.08-4.05 (t, J=7.2 Hz, 4H), 3.58 (s, 4H), 2.81 (s, 2H), 2.40-2.28 (m, 12H), 2.06-2.01 (q, J=6.8 Hz, J=14 Hz, 4H), 1.65-1.60 (m, 8H), 1.37-1.27 (m, 20H), 0.90-0.87 (t, J=6.4 Hz, 6H).

### Step 4:

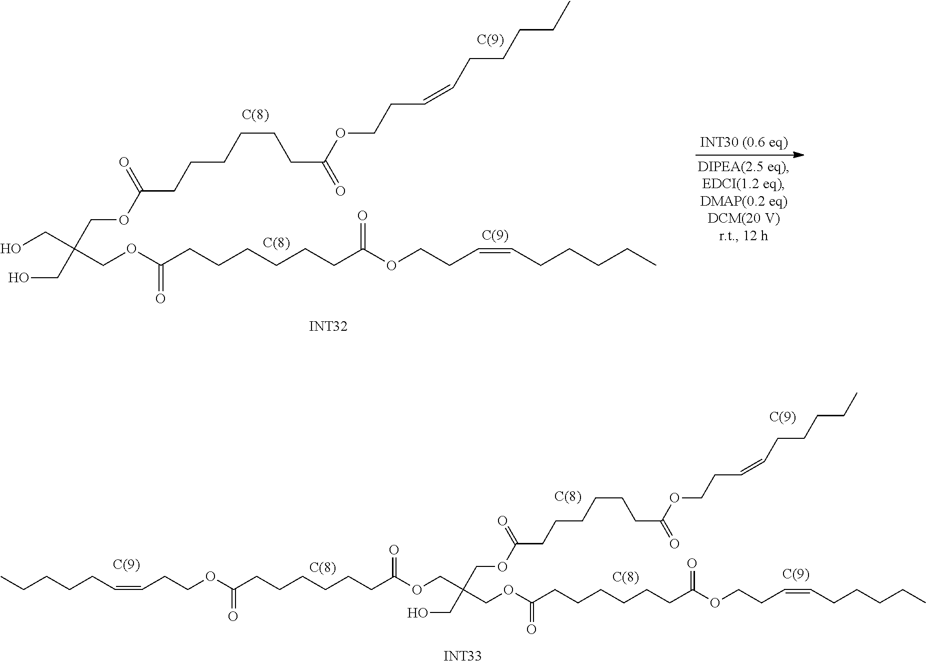

To a solution of O8-[2,2-bis(hydroxymethyl)-3-[8-[(Z)-non-3-enoxy]-8-oxo-octanoyl]oxy-propyl]O1-[(Z)-non-3-enyl]octanedioate (23 g, 33.00 mmol, 1 eq), EDCI (7.59 g, 39.60 mmol, 1.2 eq), DIPEA (10.66 g, 82.50 mmol, 14.37 mL, 2.5 eq) and DMAP (403.17 mg, 3.30 mmol, 0.1 eq) in DCM (230 mL) was added 8-[(Z)-non-3-enoxy]-8-oxo-octanoic acid (5.91 g, 19.80 mmol, 0.6 eq) in DCM (230 mL). The mixture was stirred at 20° C. for 12 hr under N₂ atmosphere. The reaction mixture was diluted with water (200 mL) and extracted with DCM 800 mL (400 mL*2). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 10/1). Compound O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (11 g, 11.25 mmol, 34.10% yield) was obtained as a colorless oil. ¹H NMR (400 MHz, CDCl3) δ ppm 5.54-5.48 (m, 3H), 5.37-5.31 (m, 3H), 4.11 (s, 6H), 4.09-4.05 (t, J=6.8 Hz, 6H), 3.51-3.50 (d, J=6.8 Hz, 2H), 2.61-2.57 (t, J=6.8 Hz, 1H), 2.40-2.28 (m, 18H), 2.07-2.02 (q, J=6.8 Hz, J=14 Hz, 6H), 1.64-1.61 (t, J=7.2 Hz, 12H), 1.38-1.27 (m, 30H), 0.91-0.88 (t, J=6.4 Hz, 9H)

### Step 5:

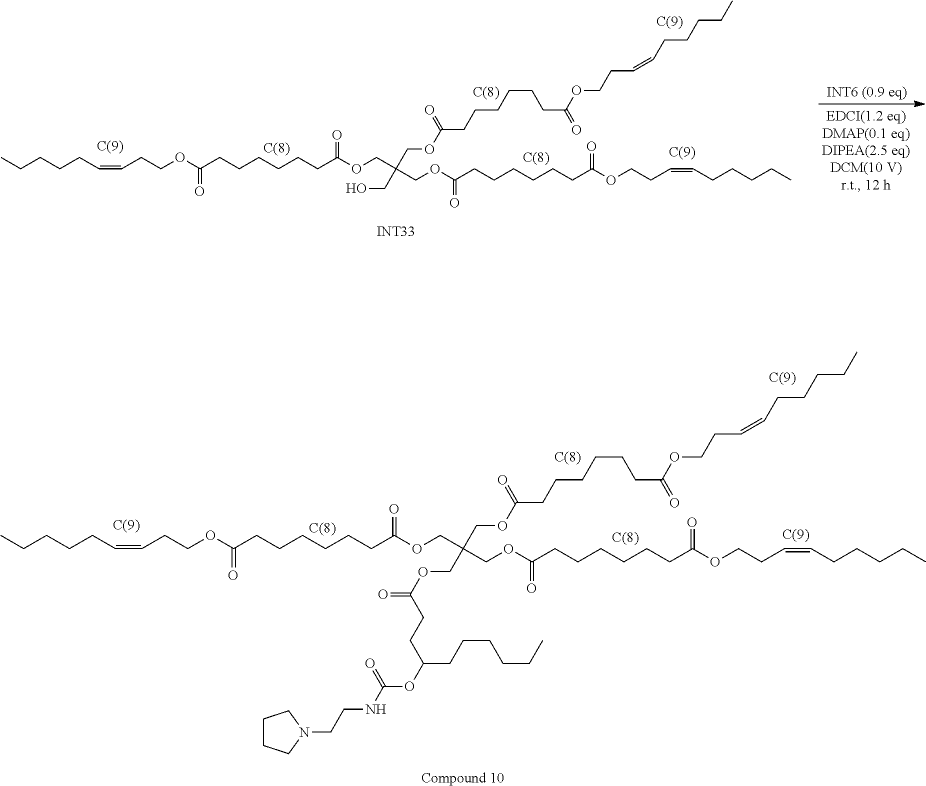

A mixture of O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (11 g, 11.25 mmol, 1 eq), 4-(2-pyrrolidin-1-ylethylcarbamoyloxy)decanoic acid (3.33 g, 10.13 mmol, 0.9 eq), EDCI (2.59 g, 13.51 mmol, 1.2 eq), DIPEA (3.64 g, 28.14 mmol, 4.90 mL, 2.5 eq) and DMAP (137.50 mg, 1.13 mmol, 0.1 eq) in DCM (100 mL) was degassed and purged with N₂ for 3 times, and then the mixture was stirred at 20° C. for 12 hr under N₂ atmosphere. The reaction mixture was diluted with water (200 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 0/1). Compound O8-[2,2-bis[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate (3.5 g, 2.72 mmol, 24.15% yield) was obtained as a colorless oil. ¹H NMR (400 MHz, CDCl₃) δ ppm 5.54-5.48 (m, 3H), 5.37-5.31 (m, 3H), 5.17 (s, 1H), 4.75 (s, 1H), 4.11 (s, 8H), 4.08-4.05 (t, J=6.8 Hz, 8H), 3.29-3.28 (d, J=4.8 Hz, 2H), 2.60-2.57 (t, J=6.0 Hz, 4H), 2.52 (s, 4H), 2.40-2.35 (q, J=7.2 Hz, J=14.0 Hz, 8H), 2.33-2.28 (q, J=7.2 Hz, J=12.4 Hz, 12H), 2.07-2.01 (q, J=6.8 Hz, J=14.0 Hz, 6H), 1.89 (s, 1H), 1.78 (s, 3H), 1.67-1.59 (m, 14H), 1.39-1.28 (m, 40H), 0.91-0.86 (m, 12H).

### Example 18—Compound 11

### O8-[2-[[2-(1-adamantyl)acetyl]oxymethyl]-2-[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate

### Step 1:

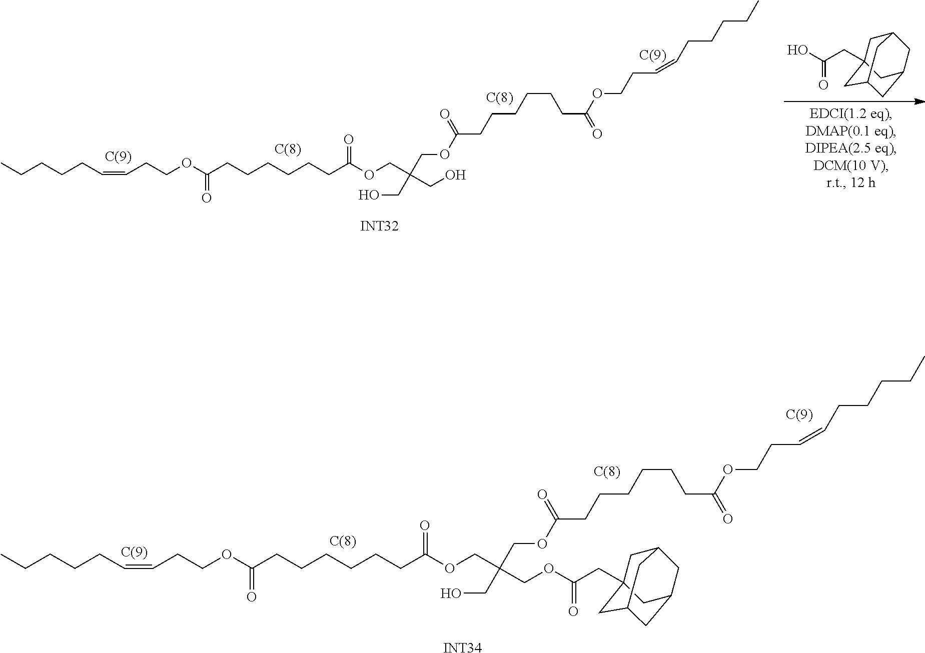

To a solution of O8-[2,2-bis(hydroxymethyl)-3-[8-[(Z)-non-3-enoxy]-8-oxo-octanoyl]oxy-propyl]O1-[(Z)-non-3-enyl]octanedioate (1 g, 1.43 mmol, 1 eq) in DCM (10 mL) was added DIPEA (463.60 mg, 3.59 mmol, 624.80 L, 2.5 eq), EDCI (330.07 mg, 1.72 mmol, 1.2 eq), DMAP (17.53 mg, 143.48 mol, 0.1 eq) and 2-(1-adamantyl)acetic acid (278.74 mg, 1.43 mmol, 1 eq). The mixture was stirred at 20° C. for 12 h under N₂. The reaction mixture was poured into H₂O (50 mL) and extracted with DCM (50 mL×3). The combined organic layers were washed with brine (50 mL), dried over Na₂SO₄, filtered and the filtrate was concentrated under reduced pressure to give a residue. The residue was purified by column chromatography (SiO₂, Petroleum ether/Ethyl acetate=8/1 to 5/1). Compound O8-[2-[[2-(1-adamantyl)acetyl]oxymethyl]-2-(hydroxymethyl)-3-[8-[(Z)-non-3-enoxy]-8-oxo-octanoyl]oxy-propyl]O1-[(Z)-non-3-enyl]octanedioate (450 mg) was obtained as a colorless oil.

### Step 2:

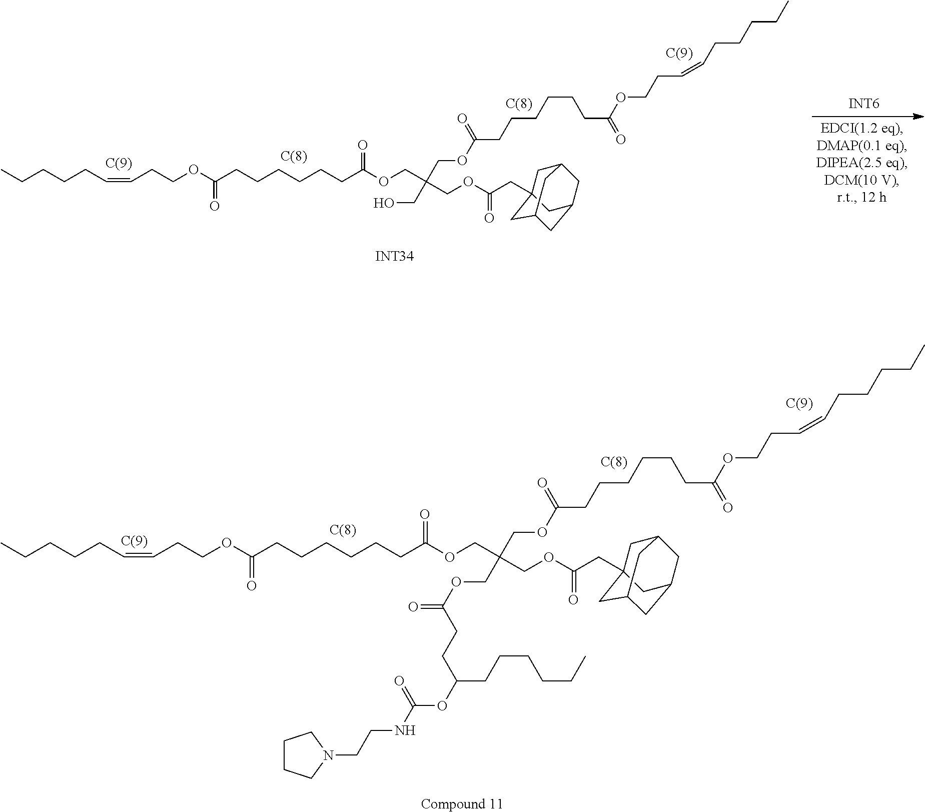

To a solution of O8-[2-[[2-(1-adamantyl)acetyl]oxymethyl]-2-(hydroxymethyl)-3-[8-[(Z)-non-3-enoxy]-8-oxooctanoyl]oxy-propyl]O1-[(Z)-non-3-enyl]octanedioate (0.65 g, 744.39 mol, 1 eq) in DCM (6.5 mL) was added EDCI (171.24 mg, 893.26 mol, 1.2 eq), DMAP (9.09 mg, 74.44 mol, 0.1 eq), DIPEA (240.52 mg, 1.86 mmol, 324.15 L, 2.5 eq) and 4-(2-pyrrolidin-1-ylethylcarbamoyloxy)decanoic acid (293.39 mg, 893.26 mol, 1.2 eq). The mixture was stirred at 15° C. for 12 h under N₂. The reaction mixture was diluted with H₂O (50 mL) and extracted with DCM 150 mL (50 mL*3). The combined organic layers were concentrated under reduced pressure to give a residue. The residue was purified by column chromatography (SiO₂, Petroleum ether/Ethyl acetate=50/1 to 2/1). Compound O8-[2-[[2-(1-adamantyl)acetyl]oxymethyl]-2-[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate (200 mg) was obtained as a colorless oil. ¹H NMR (400 MHz, CDCl₃) δ ppm 5.51-5.47 (m, 2H), 5.37-5.30 (m, 2H), 5.17 (s, 1H), 4.74 (m, 1H), 4.11-4.04 (m, 12H), 3.29- 3.28 (d, J=4.0 Hz, 2H), 2.60-2.57 (t, J=4.0 Hz, 2H), 2.52 (s, 4H), 2.39-2.34 (q, J=4.0 Hz, J=12.0 Hz, 6H), 2.32-2.27 (m, 8H), 2.07-2.00 (m, 6H), 1.96 (s, 3H), 1.88-1.87 (m, 1H), 1.78 (s, 5H), 1.77-1.68 (m, 9H), 1.62-1.57 (m, 16H), 1.35-1.26 (m, 30H), 0.90-0.86 (m, 9H).

### Example 19—Compound 12

### O8-[2-(4,4-dioctoxybutanoyloxymethyl)-2-[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate

### Step 1:

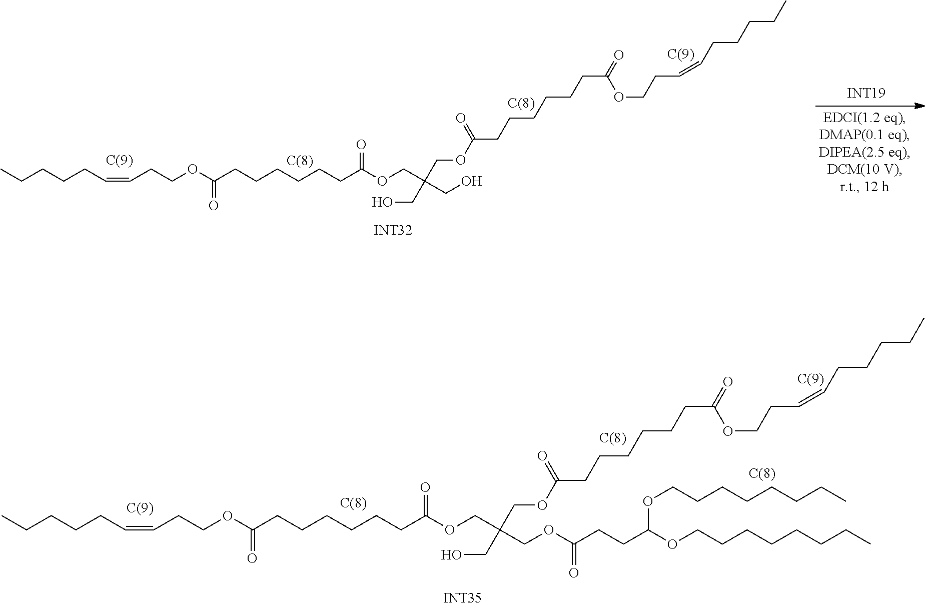

To a solution of O8-[2,2-bis(hydroxymethyl)-3-[8-[(Z)-non-3-enoxy]-8-oxo-octanoyl]oxy-propyl]O1-[(Z)-non-3-enyl]octanedioate (1.5 g, 2.15 mmol, 1 eq) in DCM (15 mL) was added DIPEA (695.40 mg, 5.38 mmol, 937.20 L, 2.5 eq), EDCI (495.11 mg, 2.58 mmol, 1.2 eq), DMAP (26.29 mg, 215.22 mol, 0.1 eq) and 4,4-dioctoxybutanoic acid (444.90 mg, 1.29 mmol, 0.6 eq). The mixture was stirred at 20° C. for 12 h under N₂. The reaction mixture was poured into H₂O (50 mL) and extracted with DCM (50 mL×3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=8/1 to 5/1). Compound O8-[2-(4,4-dioctoxybutanoyloxymethyl)-2-(hydroxymethyl)-3-[8-[(Z)-non-3 -enoxy]-8-oxo-octanoyl]oxy-propyl]O1-[(Z)-non-3-enyl]octanedioate (0.7 g) was obtained as a colorless oil.

### Step 2:

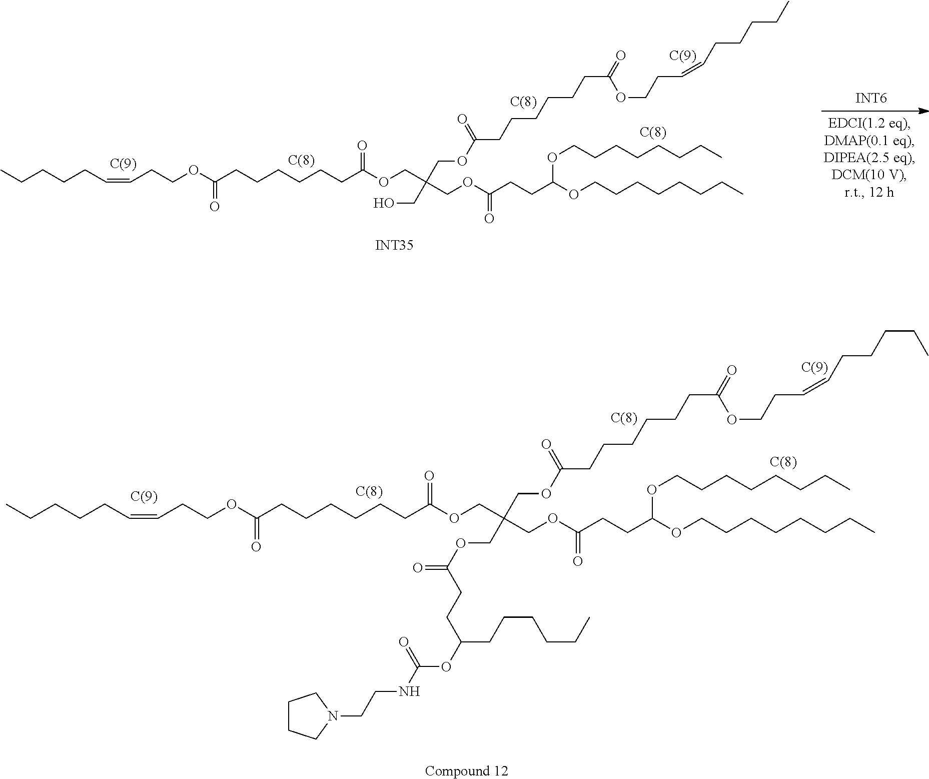

To a solution of O8-[2-(4,4-dioctoxybutanoyloxymethyl)-2-(hydroxymethyl)-3-[8-[(Z)-non-3-enoxy]-8-oxo-octanoyl]oxy-propyl]O1-[(Z)-non-3-enyl]octanedioate (700 mg, 683.95 mol, 1 eq) in DCM (7 mL) was added DIPEA (220.99 mg, 1.71 mmol, 297.83 L, 2.5 eq), EDCI (157.34 mg, 820.74 mol, 1.2 eq), DMAP (8.36 mg, 68.40 mol, 0.1 eq) and 4-(2-pyrrolidin-1-ylethylcarbamoyloxy)decanoic acid (269.57 mg, 820.74 mol, 1.2 eq). The mixture was stirred at 20° C. for 12 h under N₂. The reaction mixture was poured into H₂O (50 mL) and extracted with DCM (50 mL×3). The combined organic layers were washed with brine (50 mL), dried over Na₂SO₄, filtered and the filtrate was concentrated under reduced pressure to give a residue. The residue was purified by column chromatography (SiO₂, Petroleum ether/Ethyl acetate=8/1 to 5/1). Compound O8-[2-(4,4-dioctoxybutanoyloxymethyl)-2-[[8-[(Z)-non-3-enoxy]-8-oxo-octanoyl]oxymethyl]-3-[4-(2-pyrrolidin-1-ylethylcarbamoyloxy)decanoyloxy]propyl]O1-[(Z)-non-3-enyl]octanedioate (160 mg) was obtained as a colorless oil. ¹H NMR (400 MHz, CDCl₃) δ=5.54-5.48 (m, 2H), 5.38-5.31 (m, 2H), 5.17 (s, 1H), 4.75 (s, 1H), 4.50-4.47 (t, J=8.0 Hz, 1H), 4.12-4.11 (d, J= 4.0 Hz, 8H), 4.09-4.05 (t, J=6.8 Hz, 4H), 3.59-3.53 (m, 2H), 3.43-3.38 (m, 2H), 3.30-3.28 (m, 2H), 2.61-2.58 (t, J=6.0 Hz, 2H), 2.52 (s, 4H), 2.42-2.35 (m, 8H), 2.33-2.28 (m, 8H), 2.07-2.02 (q, J=6.8 Hz, J=14 Hz, 4H), 1.94-1.89 (m, 3H), 1.78 (s, 5H), 1.64-1.55 (m, 14H), 1.38-1.28 (m, 48H), 0.91-0.87 (m, 15H)

### Example 20—Compound 13

### O6-[2,2-bis[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Step 1:

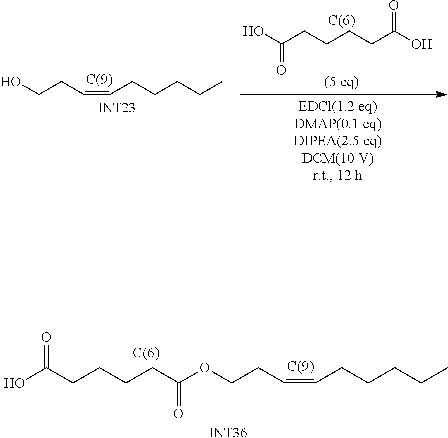

To a solution of (Z)-non-3-en-1-ol (30.00 g, 210.91 mmol, 1 eq) in DCM (300 mL) was added EDCI (48.52 g, 253.10 mmol, 1.2 eq), DIPEA (68.15 g, 527.29 mmol, 91.84 mL, 2.5 eq), adipic acid (154.12 g, 1.05 mol, 175.13 mL, 5 eq) and DMAP (2.58 g, 21.09 mmol, 0.1 eq). The mixture was stirred at 20° C. for 12 hr under N₂. The reaction mixture was diluted with H₂O (300 mL) and extracted with DCM 900 mL (300 mL*3). The combined organic layers were concentrated under reduced pressure to give a residue. The residue was purified by column chromatography (SiO₂, Petroleum ether/Ethyl acetate=100/1 to 0/1). Compound 6-[(Z)-non-3-enoxy]-6-oxo-hexanoic acid (30 g, 110.96 mmol, 52.61% yield) was obtained as a colorless oil. ¹H NMR (400 MHz, CDCl₃) δ ppm 5.54-5.50 (m, 1H), 5.35-5.32 (m, 1H), 4.09-4.06 (t, J=7.2 Hz, 2H), 2.04-2.32 (m, 6H), 2.06-2.01 (m, 2H), 1.70-1.67 (m, 4H), 1.37-1.26 (m, 6H), 0.91-0.88 (t, J=6.8 Hz, 3H)

### Step 2:

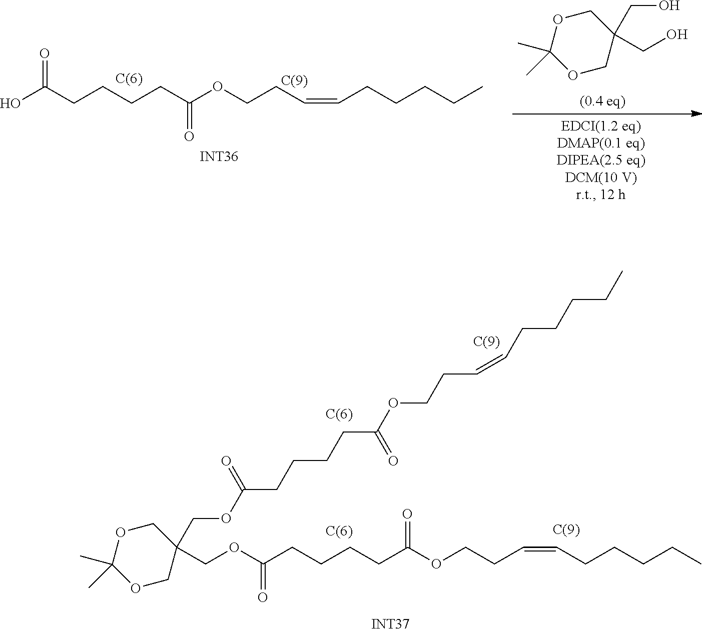

To a solution of 6-[(Z)-non-3-enoxy]-6-oxo-hexanoic acid (26.85 g, 99.31 mmol, 1 eq) in DCM (270 mL) was added EDCI (22.85 g, 119.18 mmol, 1.2 eq), DIPEA (32.09 g, 248.28 mmol, 43.25 mL, 2.5 eq), [5-(hydroxymethyl)-2,2-dimethyl-1,3-dioxan-5-yl]methanol (7 g, 39.73 mmol, 0.4 eq) and DMAP (1.21 g, 9.93 mmol, 0.1 eq). The mixture was stirred at 20° C. for 12 hr under N₂. The reaction mixture was diluted with H₂O (300 mL) and extracted with DCM 900 mL (300 mL*3). The combined organic layers were concentrated under reduced pressure to give a residue. The residue was purified by column chromatography (SiO₂, Petroleum ether/Ethyl acetate=100/1 to 0/1). Compound O6-[[2,2-dimethyl-5-[[6-[(Z)-non-3-enoxy]-6-oxo-hexanoyl]oxymethyl]-1,3-dioxan-5-yl]methyl]O1-[(Z)-non-3-enyl]hexanedioate (15 g, 22.03 mmol, 22.18% yield) was obtained as a colorless oil. ¹H NMR (400 MHz, CDCl₃) δ ppm 5.52-5.49 (m, 2H), 5.35-5.30 (m, 2H), 4.11 (S, 1H), 4.09-4.05 (t, J=7.2 Hz, 4H), 3.74 (s, 4H), 2.40-2.30 (m, 12H), 2.06-2.01 (q, J=6.8 Hz, J=14 Hz, 4H), 1.67-1.64 (m, 8H), 1.42 (s, 6H), 1.37-1.26 (m, 12H), 0.91-0.88 (t, J=6.4 Hz, 6H).

### Step 3:

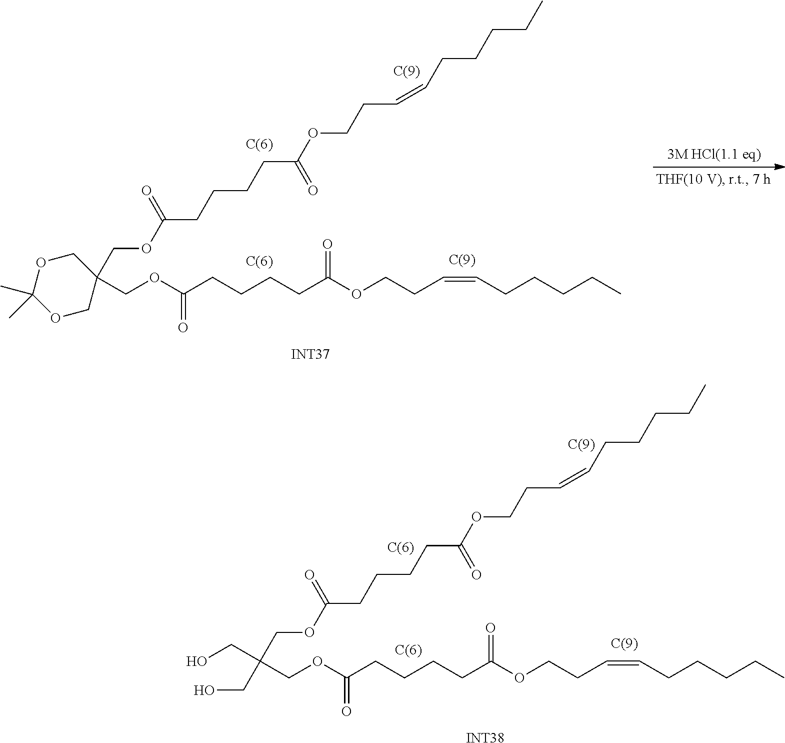

To a solution of O6-[[2,2-dimethyl-5-[[6-[(Z)-non-3-enoxy]-6-oxo-hexanoyl]oxymethyl]-1,3-dioxan-5-yl]methyl]O1-[(Z)-non-3-enyl]hexanedioate (15 g, 22.03 mmol, 1 eq) in THF (150 mL) was added HCl (3 M, 8.08 mL, 1.1 eq) slowly. The mixture was stirred at 20° C. for 7 hr under N₂. The reaction mixture was diluted with sat.NaHCO₃ (300 mL) and extracted with DCM 900 mL (300 mL*3). The combined organic layers were concentrated under reduced pressure to give a residue. The residue was purified by column chromatography (SiO₂, Petroleum ether/Ethyl acetate=100/1 to 0/1). Compound O6-[2,2-bis(hydroxymethyl)-3-[6-[(Z)-non-3-enoxy]-6-oxo-hexanoyl]oxy-propyl]O1-[(Z)-non-3-enyl]hexanedioate (6 g, 9.36 mmol, 42.50% yield) was obtained as a colorless oil.

### Step 4:

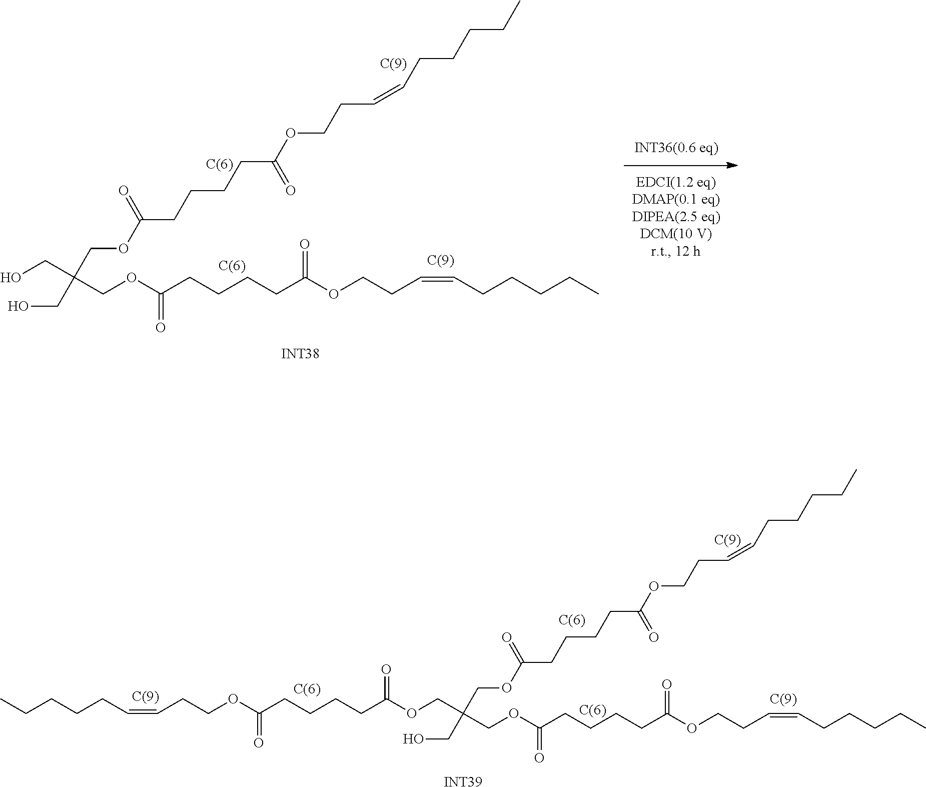

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (20 mL) was added EDCI (717.93 mg, 3.75 mmol, 1.2 eq), DIPEA (1.01 g, 7.80 mmol, 1.36 mL, 2.5 eq), 6-[(Z)-non-3-enoxy]-6-oxo-hexanoic acid (506.27 mg, 1.87 mmol, 0.6 eq) and DMAP (38.13 mg, 312.09 mol, 0.1 eq). The mixture was stirred at 20° C. for 12 hr under N₂. The reaction mixture was diluted with H₂O (30 mL) and extracted with DCM 90 mL (30 mL*3). The combined organic layers were concentrated under reduced pressure to give a residue. The residue was purified by column chromatography (SiO₂, Petroleum ether/Ethyl acetate=100/1 to 0/1). Compound O6-[2-(hydroxymethyl)-3-[6-[(Z)-non-3-enoxy]-6-oxo-hexanoyl]oxy-2-[[6-[(Z)-non-3-enoxy]-6-oxo-hexanoyl]oxymethyl]propyl]O1-[(Z)-non-3-enyl]hexanedioate (0.9 g, 1.01 mmol, 32.29% yield) was obtained as a colorless oil. ¹H NMR (400 MHz, CDCl₃) δ ppm 5.54-5.48 (m, 3H), 5.37-5.32 (m, 3H), 4.12 (s, 6H), 4.09-4.05 (t, J=7.2 Hz, 6H), 3.53-3.51 (d, J=6.4 Hz, 2H), 2.69-2.67 (t, J=6.4 Hz, 1H), 2.40- 2.31 (m, 18H), 2.07-2.01 (m, 6H), 1.68-1.64 (m, 12H), 1.38-1.27 (m, 18H), 0.91-0.88 (t, J=6.8 Hz, 9H)

### Step 5:

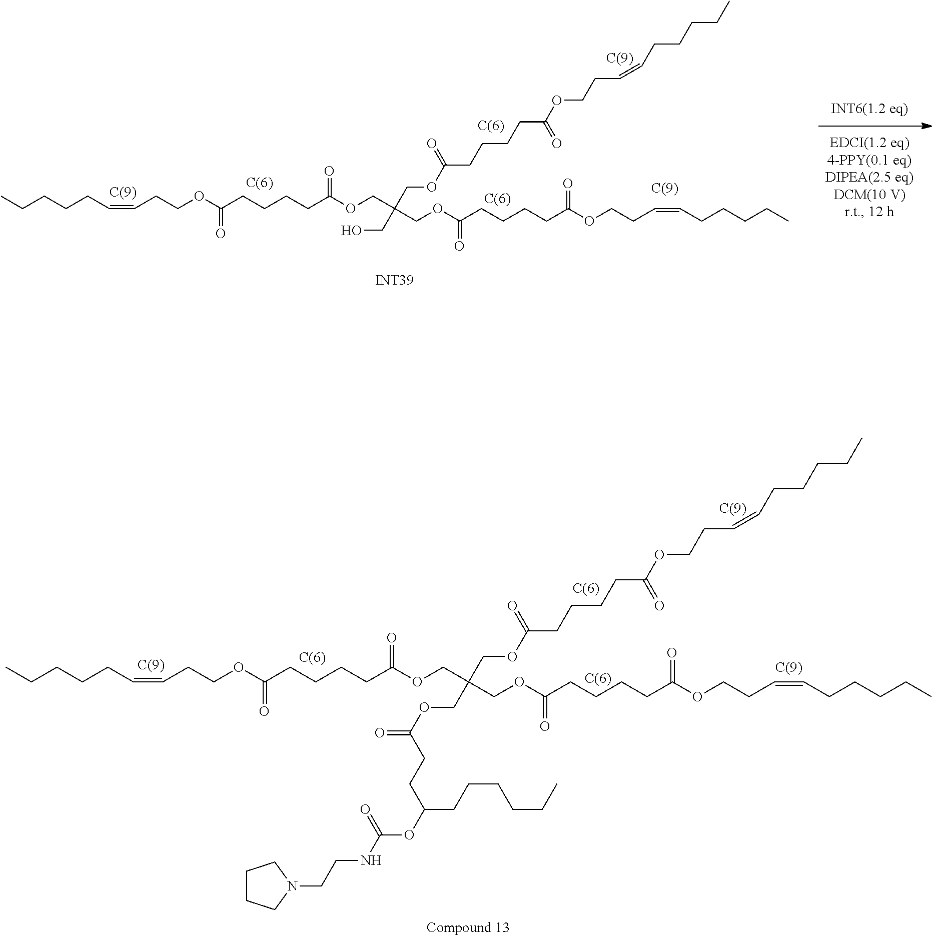

To a solution of O6-[2-(hydroxymethyl)-3-[6-[(Z)-non-3-enoxy]-6-oxo-hexanoyl]oxy-2-[[6-[(Z)-non-3-enoxy]-6-oxo-hexanoyl]oxymethyl]propyl]O1-[(Z)-non-3-enyl]hexanedioate (0.9 g, 1.01 mmol, 1 eq) in DCM (9 mL) was added EDCI (231.79 mg, 1.21 mmol, 1.2 eq), DIPEA (325.57 mg, 2.52 mmol, 438.77 L, 2.5 eq), 4-(2-pyrrolidin-1-ylethylcarbamoyloxy)decanoic acid (397.14 mg, 1.21 mmol, 1.2 eq) and 4-pyrrolidin-1-ylpyridine (14.93 mg, 100.76 mol, 0.1 eq). The mixture was stirred at 20° C. for 12 hr under N₂. The reaction mixture was diluted with H₂O (30 mL) and extracted with DCM 90 mL (30 mL*3). The combined organic layers were concentrated under reduced pressure to give a residue. The residue was purified by column chromatography (SiO₂, Petroleum ether/Ethyl acetate=100/1 to 0/1). Compound O6-[2,2-bis[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.5 g, 415.41 mol, 41.23% yield) was obtained as a colorless oil. ¹H NMR (400 MHz, CDCl₃) δ ppm 5.52-5.48 (m, 3H), 5.37-5.19 (m, 3H), 5.19 (s, 1H), 4.75 (s, 1H), 4.11 (s, 8H), 4.09-4.05 (t, J=6.8 Hz, 6H), 3.23-3.29 (d, J=4 Hz, 2H), 2.60 (s, 2H), 2.52 (s, 4H), 2.40-2.31 (m, 20H), 2.07-2.01 (q, J=6.8 Hz, 6H), 1.92-1.85 (m, 1H), 1.78 (s, 5H), 1.67-1.63 (m, 14H), 1.38-1.27 (m, 26H), 0.91-0.86 (m, 12H)

### Example 21—Compound 14

### O6-[2-[[2-(1-adamantyl) acetyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Step 1:

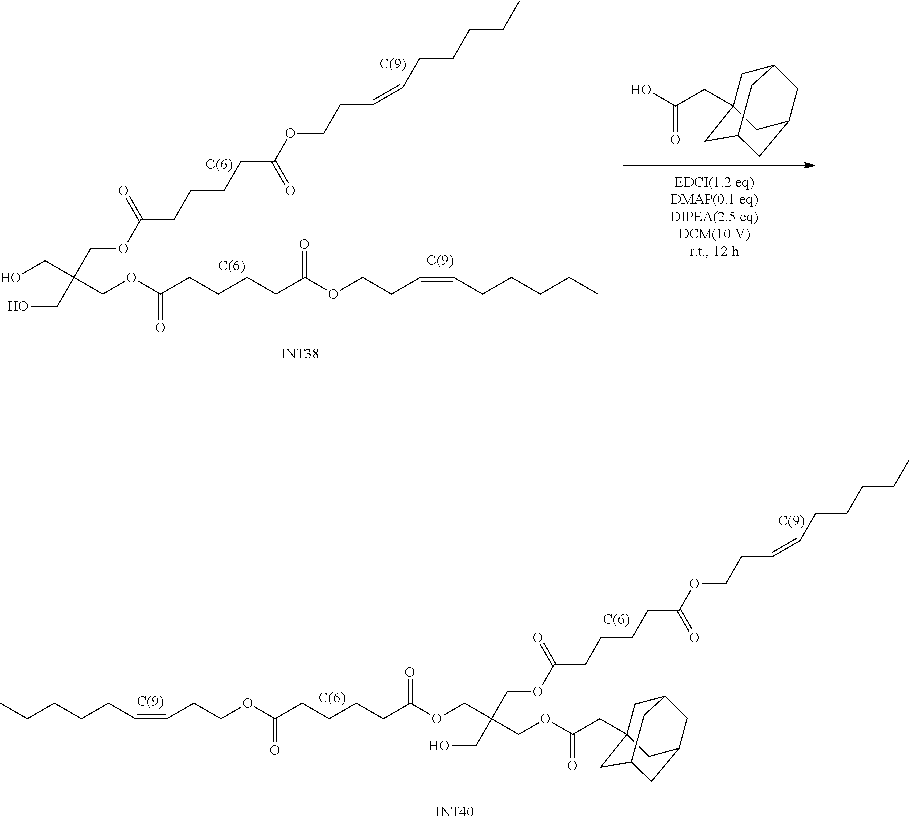

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (20 mL) was added EDCI (717.94 mg, 3.75 mmol, 1.2 eq), DIPEA (1.01 g, 7.80 mmol, 1.36 mL, 2.5 eq), 2-(1-adamantyl)acetic acid (363.78 mg, 1.87 mmol, 0.6 eq) and DMAP (38.13 mg, 312.09 mol, 0.1 eq). The mixture was stirred at 20° C. for 12 hr under N₂. The reaction mixture was diluted with H₂O (100 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound O6-[2-[[2-(1-adamantyl) acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 979.08 mol, 31.37% yield) was obtained as a colorless oil. ¹H NMR (400 MHz, CDCl₃) δ ppm 5.52-5.48 (m, 2H), 5.37-5.31 (m, 2H), 4.12 (s, 4H), 4.09-4.06 (m, 6H), 5.53-5.52 (d, J=6.8 Hz, 2H), 2.63-2.59 (t, J=6.8 Hz, 1H), 2.40-2.31 (m, 12H), 2.10 (s, 2H), 2.07-2.01 (q, J=6.4 Hz, J= 14 Hz, 4H), 1.98 (s, 3H), 1.73-1.59 (m, 20H), 1.38-1.27 (m, 12H), 0.91-0.88 (t, J=6.4 Hz, 6H)

### Step 2:

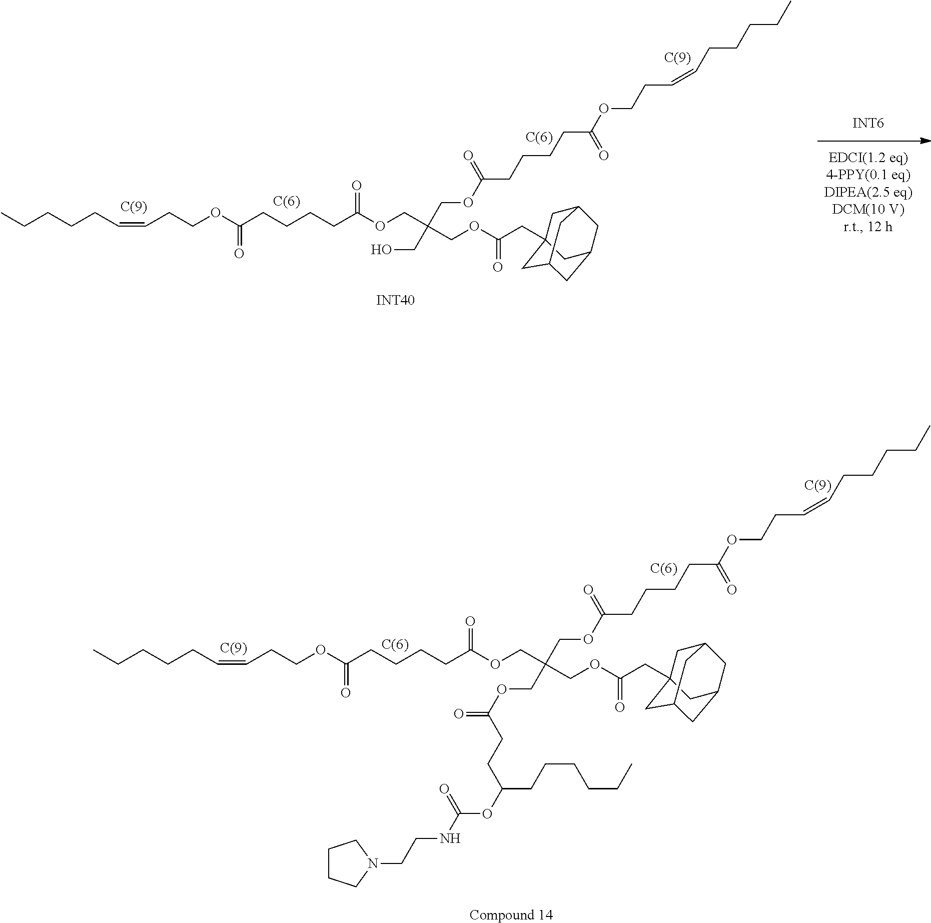

To a solution of O6-[2-[[2-(1-adamantyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 979.08 mol, 1.1 eq) in DCM (8 mL) was added EDCI (204.75 mg, 1.07 mmol, 1.2 eq), 4-(2-pyrrolidin-1-ylethylcarbamoyloxy)decanoic acid (292.34 mg, 890.07 mol, 1 eq), DIPEA (287.59 mg, 2.23 mmol, 387.59 μL, 2.5 eq) and DMAP (10.87 mg, 89.01 mol, 0.1 eq). The mixture was stirred at 20° C. for 12 hr under N₂. The reaction mixture was diluted with H₂O (10 mL) and extracted with DCM 30 mL (10 mL*3). The combined organic layers were concentrated under reduced pressure to give a residue. The residue was purified by column chromatography (SiO₂, Petroleum ether/Ethyl acetate=100/1 to 10/1). Compound O6-[2-[[2-(1-adamantyl) acetyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.1 g, 88.69 mol, 9.96% yield) was obtained as a colorless oil. ¹H NMR (400 MHz, CDCl₃) δ ppm 5.52-5.48 (m, 2H), 5.35-5.31 (m, 2H), 5.21 (s, 1H), 4.75 (s, 1H), 4.12 (s, 6H), 4.09-4.05 (t, J=6.8 Hz, 6H), 3.31-3.30 (d, J=3.2 Hz, 2H), 2.62-2.55 (d, J=30 Hz, 6H), 2.40-2.31 (m, 14H), 2.08-2.01 (m, 6H), 1.97 (s, 3H), 1.88 (s, 1H), 1.80 (s, 5H), 1.72-1.63 (m, 14H), 1.60-1.58 (m, 8H), 1.40-1.27 (m, 20H), 0.91-0.86 (m, 9H)

### Example 22—Compound 15

### O6-[2-(4,4-dioctoxybutanoyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Step 1:

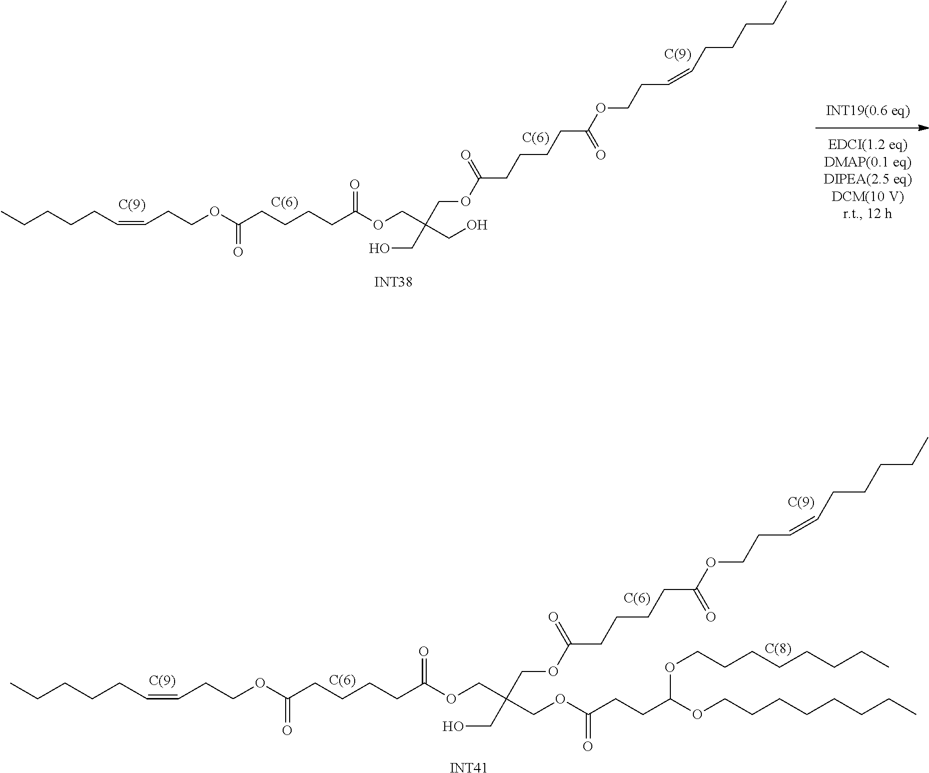

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (20 mL) was added EDCI (717.93 mg, 3.75 mmol, 1.2 eq), 4,4-dioctoxybutanoic acid (645.14 mg, 1.87 mmol, 0.6 eq), DIPEA (1.01 g, 7.80 mmol, 1.36 mL, 2.5 eq) and DMAP (38.13 mg, 312.09 mol, 0.1 eq). The mixture was stirred at 20° C. for 12 hr under N₂. The reaction mixture was diluted with H₂O (100 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound O6-[2-(4,4-dioctoxybutanoyloxymethyl)-2-(hydroxymethyl)-3-[6-[(Z)-non- 3-enoxy]-6-oxo-hexanoyl]oxy-propyl]O1-[(Z)-non-3-enyl]hexanedioate (0.6 g, 620.25 mol, 19.87% yield) was obtained as a colorless oil.

### Step 2:

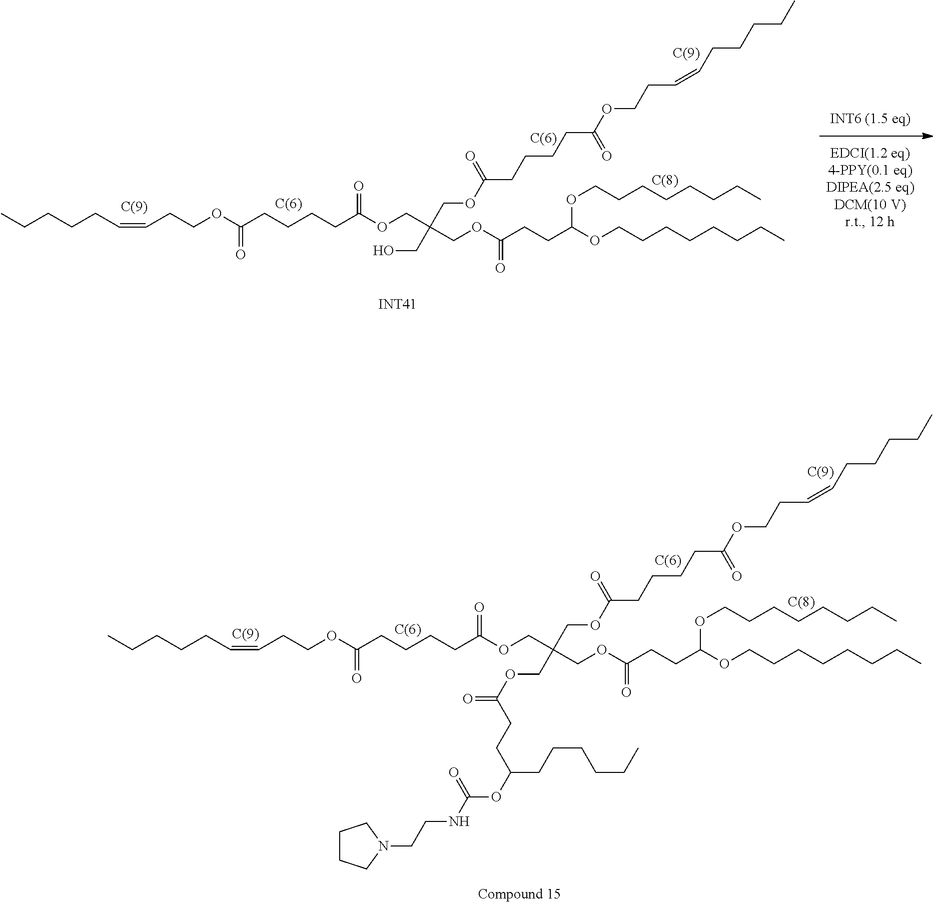

To a solution of O6-[2-(4,4-dioctoxybutanoyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.6 g, 620.25 mol, 1 eq) in DCM (6 mL) was added EDCI (142.68 mg, 744.30 mol, 1.2 eq), 4-(2-pyrrolidin-1-ylethylcarbamoyloxy)decanoic acid (305.58 mg, 930.37 mol, 1.5 eq), DIPEA (200.41 mg, 1.55 mmol, 270.09 L, 2.5 eq) and 4-pyrrolidin-1-ylpyridine (9.19 mg, 62.02 mol, 0.1 eq). The mixture was stirred at 20° C. for 12 hr under N₂. The reaction mixture was diluted with H₂O (10 mL) and extracted with DCM 30 mL (10 mL*3). The combined organic layers were concentrated under reduced pressure to give a residue. The residue was purified by column chromatography (SiO₂, Petroleum ether/Ethyl acetate=100/1 to 0/1). Compound O6-[2-(4,4-dioctoxybutanoyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[ (Z)-non-3-enyl]hexanedioate (0.15 g, 117.39 mol, 18.93% yield) was obtained as a colorless oil. ¹H NMR (400 MHz, CDCl₃) δ ppm 5.54-5.48 (m, 2H), 5.35-5.33 (m, 2H), 5.19 (s, 1H), 4.75 (s, 1H), 4.50-4.47 (t, J=5.6 Hz, 1H), 4.11 (s, 8H), 4.09-4.05 (t, J=7.2 Hz, 4H), 3.57-3.53 (m, 2H), 3.43-3.38 (m, 2H), 3.30-3.28 (d, J=5.2 Hz, 2H), 2.61-2.58 (t, J=5.6 Hz, 2H), 2.52 (s, 4H), 2.40-2.32 (m, 14H), 2.07-2.02 (q, J=6.4 Hz, J=14 Hz, 4H), 1.94-1.89 (q, J=7.6 Hz, J=13.6 Hz, 3H), 1.78 (s, 5H), 1.69-1.63 (m, 10H), 1.60-1.53 (m, 6H), 1.36-1.28 (m, 40H), 0.91-0.87 (m, 15H)

### Example 23—Compound 16

### O7-[2-[7-(2-butyloctanoyloxy)heptanoyloxymethyl]-2-[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate

### Step 1:

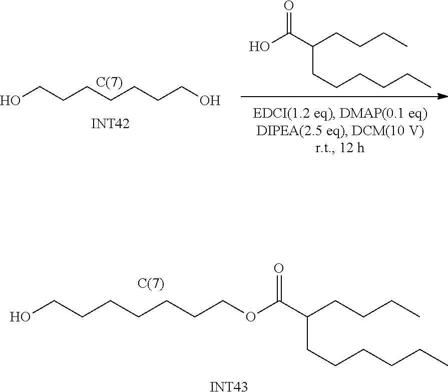

To a solution of heptane-1,7-diol (49.50 g, 374.41 mmol, 3 eq) in DCM (500 mL) was added DIPEA (40.32 g, 312.01 mmol, 54.35 mL, 2.5 eq), 2-butyloctanoic acid (25 g, 124.80 mmol, 1 eq), DMAP (1.52 g, 12.48 mmol, 0.1 eq) and EDCI (28.71 g, 149.76 mmol, 1.2 eq). The mixture was stirred at 25° C. for 12 hr under N₂. The reaction mixture was diluted with H₂O (600 mL) and extracted with DCM 1000 mL (500 mL*2). The combined organic layers were dried over Na₂SO₄, filtered and the filtrate was concentrated under reduced pressure to give a residue. Compound 7-hydroxyheptyl 2-butyloctanoate (100 g, 317.96 mmol, 63.69% yield) was obtained as a colorless oil.

### Step 2:

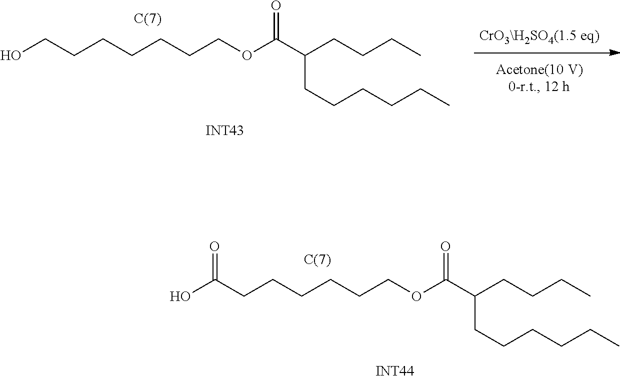

To a solution of 7-hydroxyheptyl 2-butyloctanoate (25 g, 79.49 mmol, 1 eq) in acetone (250 mL) was added CrO₃\H₂SO₄ (238.47 mg, 119.24 mmol, 1.5 eq) dropwise at 0° C. The mixture was stirred at 25° C. for 12 hr under N₂. The reaction mixture was diluted with H₂O (200 mL) and extracted with EtOAc 300 mL (100 mL*3). The combined organic layers were dried over Na₂SO₄, filtered and the filtrate was concentrated under reduced pressure to give a residue. Compound 7-(2-butyloctanoyloxy)heptanoic acid (50 g, 152.21 mmol, 47.87% yield) was obtained as a colorless oil. ¹H NMR (400 MHz, CDCl₃) δ ppm 11.27-10.80 (m, 1H), 4.09-4.06 (t, J=6.8 Hz, 2H), 2.39-2.28 (m, 3H), 1.57-1.55 (m, 6H), 1.47-1.37 (m, 6H), 1.33-1.22 (m, 12H), 0.90-0.86 (m, 6H)

### Step 3:

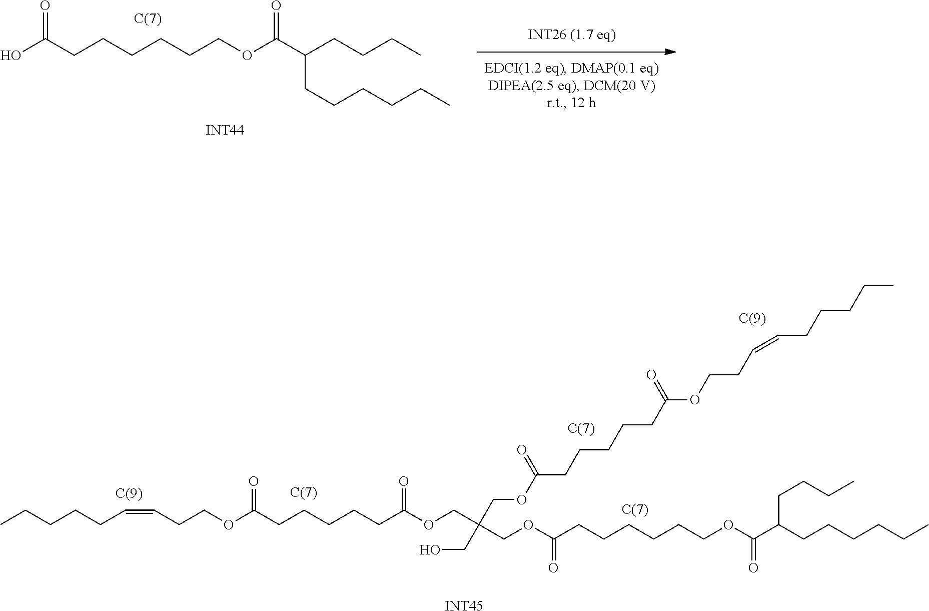

To a solution of O7-[2,2-bis(hydroxymethyl)-3-[7-[(Z)-non-3-enoxy]-7-oxo-heptanoyl]oxy-propyl]O1-[(Z)-non-3-enyl]heptanedioate (2 g, 2.99 mmol, 1 eq) in DCM (20 mL) was added EDCI (687.83 mg, 3.59 mmol, 1.2 eq), DIPEA (966.09 mg, 7.48 mmol, 1.30 mL, 2.5 eq), 7-(2-butyloctanoyloxy)heptanoic acid (589.30 mg, 1.79 mmol, 0.6 eq) and DMAP (36.53 mg, 299.00 mol, 0.1 eq). The mixture was stirred at 20° C. for 12 hr under N₂. The reaction mixture was diluted with H₂O (20 mL) and extracted with DCM 30 mL (10 mL*3). The combined organic layers were concentrated under reduced pressure to give a residue. The residue was purified by column chromatography (SiO₂, Petroleum ether/Ethyl acetate=100/1 to 0/1). Compound O7-[2-[7-(2-butyloctanoyloxy)heptanoyloxymethyl]-2-(hydroxymethyl)-3-[7-[(Z)-non-3-enoxy]-7-oxo-heptanoyl]oxy-propyl]O1-[(Z)-non-3-enyl]heptanedioate (1 g, 1.02 mmol, 34.15% yield) was obtained as a colorless oil. ¹H NMR (400 MHz, CDCl₃) δ ppm 5.52-5.48 (m, 2H), 5.37-5.33 (m, 2H), 4.12 (s, 6H), 4.09-4.05 (t, J=6.8 Hz, 6H), 3.52-3.50 (d, J=6.8 Hz, 2H), 2.64-2.61 (t, J=6.8 Hz, 1H), 2.39-2.29 (m, 14H), 2.07- 2.02 (q, J=6.4 Hz, J=10.8 Hz, 4H), 1.68-1.57 (m, 16H), 1.47-1.43 (m, 2H), 1.38-1.26 (m, 30H), 0.91-0.86 (m, 12H)

### Step 4:

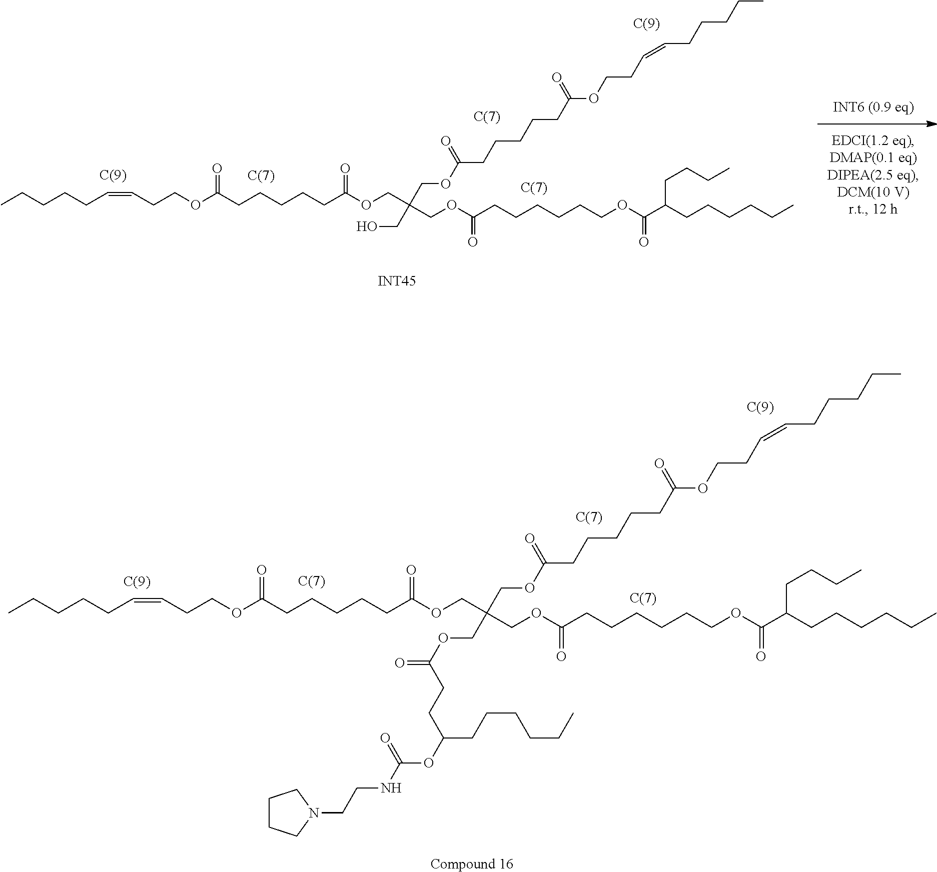

To a solution of O7-[2-[7-(2-butyloctanoyloxy)heptanoyloxymethyl]-2-(hydroxymethyl)-3-[7-[(Z)-non-3-enoxy]-7-oxo-heptanoyl]oxy-propyl]O1-[(Z)-non-3-enyl]heptanedioate (1 g, 1.02 mmol, 1 eq) in DCM (10 mL) was added EDCI (234.89 mg, 1.23 mmol, 1.2 eq), DIPEA (329.92 mg, 2.55 mmol, 444.63 μL, 2.5 eq), 4-(2-pyrrolidin-H-ylethyl carbamoyloxy)decanoic acid (301.83 mg, 918.96 μmol, 0.9 eq) and DMAP (12.47 mg, 102.11 μmol, 0.1 eq). The mixture was stirred at 20° C. for 12 hr under N₂. The reaction mixture was diluted with H₂O (20 mL) and extracted with DCM 30 mL (10 mL*3). The combined organic layers were concentrated under reduced pressure to give a residue. The residue was purified by column chromatography (SiO₂, Petroleum ether/Ethyl acetate=100/1 to 0/1). Compound O7-[2-[7-(2-butyloctanoyloxy)heptanoyloxymethyl]-2-[[7-[(Z)-non-3-enoxy]-7-oxo-heptanoyl]oxymethyl]-3-[4-(2-pyrrolidin-1-ylethylcarbamoyloxy)decanoyloxy]propyl]O1-[(Z)-non-3-enyl]heptanedioate (0.15 g, 116.30 μmol, 11.3900 yield) was obtained as a colorless oil. ¹H NMR (400 MHz, CDCl₃) δ ppm 5.52-5.48 (m, 2H), 5.37-5.31 (m, 2H), 5.27-5.01 (m, 1H), 4.75 (s, 1H), 4.11 (s, 8H), 4.08-4.05 (t, J=6.8 Hz, 6H), 3.31 (s, 2H), 2.62-2.55 (m, 6H), 2.40-2.29 (m, 18H), 2.07-2.02 (q, J=6.4 Hz, J=14 Hz, 4H), 1.91-1.80 (m, 5H), 1.66-1.59 (m, 14H), 1.46-1.27 (m, 44H), 0.91-0.86 (m, 15H)

### Example 24—Compound 17

### O7-[2,2-bis[7-(2-butyloctanoyloxy)heptanoyloxymethyl]-3-[4-(2-pyrrolidin-1-ylethylcarbamoyloxy)decanoyloxy]propyl]O1-[(Z)-non-3-enyl]heptanedioate

### Step 1:

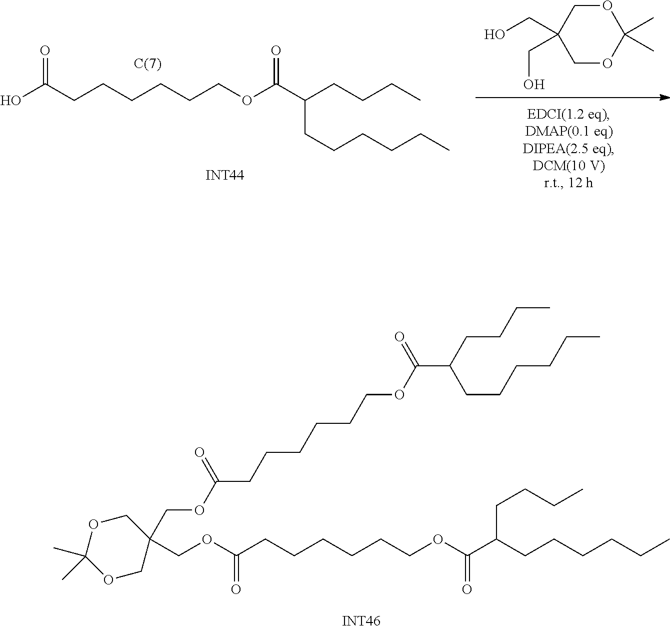

To a solution of 7-(2-butyloctanoyloxy)heptanoic acid (24.86 g, 75.67 mmol, 1 eq) in DCM (250 mL) was added EDCI (17.41 g, 90.80 mmol, 1.2 eq), [5-(hydroxymethyl)-2,2-dimethyl-1,3-dioxan-5-yl]methanol (6 g, 34.05 mmol, 0.45 eq), DMAP (924.41 mg, 7.57 mmol, 0.1 eq) and DIPEA (24.45 g, 189.17 mmol, 32.95 mL, 2.5 eq). The mixture was stirred at 25° C. for 12 hr under N₂. The mixture was diluted with H₂O (300 mL) and extracted with DCM 500 mL (100 mL*5). The combined organic layers were dried over Na₂SO₄, filtered and the filtrate was concentrated under reduced pressure to give a residue. Compound [7-[[5-[7-(2-butyloctanoyloxy)heptanoyloxymethyl]-2,2-dimethyl-1,3-dioxan-5-yl]methoxy]-7-oxo-heptyl]2-butyloctanoate (13 g, 16.31 mmol, 21.55% yield) was obtained as a colorless oil.

### Step 2:

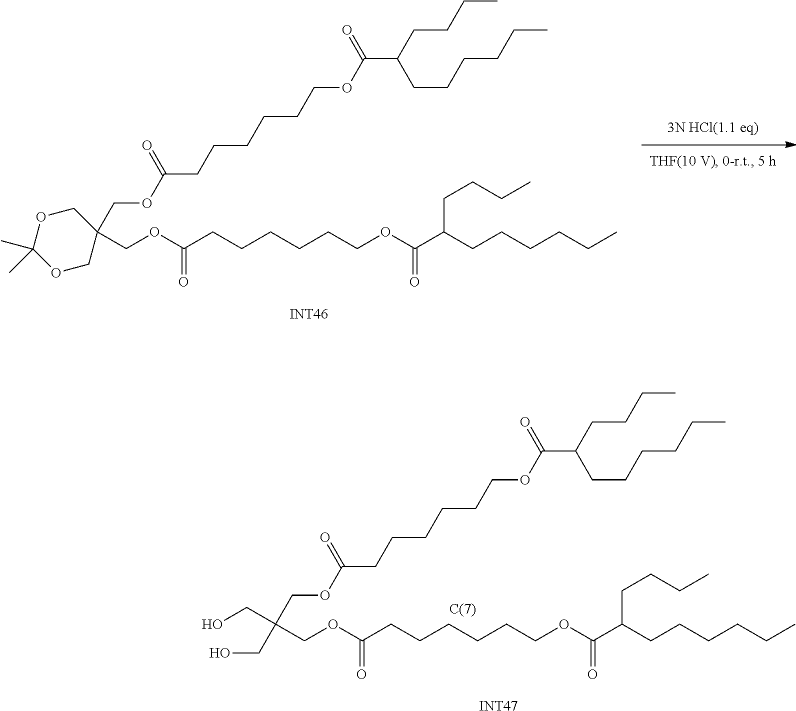

To a solution of [7-[[5-[7-(2-butyloctanoyloxy) heptanoyloxymethyl]-2,2-dimethyl-1,3-dioxan-5-yl]methoxy]-7-oxo-heptyl]2-butyloctanoate (27 g, 33.87 mmol, 1 eq) in THF (270 mL) was added HCl (3 M, 12.42 mL, 1.1 eq) dropwise at 0° C. The mixture was stirred at 25° C. for 5 hr under N₂. Then it was poured into H₂O (200 mL) and extracted with DCM 800 mL (200 mL*4). The combined organic layers were dried over Na₂SO₄, filtered and the filtrate was concentrated under reduced pressure to give a residue. Compound [7-[2-[7-(2-butyloctanoyloxy) heptanoyloxymethyl]-3-hydroxy-2-(hydroxymethyl) propoxy]-7-oxo-heptyl]2-butyloctanoate (10 g, 13.21 mmol, 39.00% yield) was obtained as a colorless oil.

### Step 3:

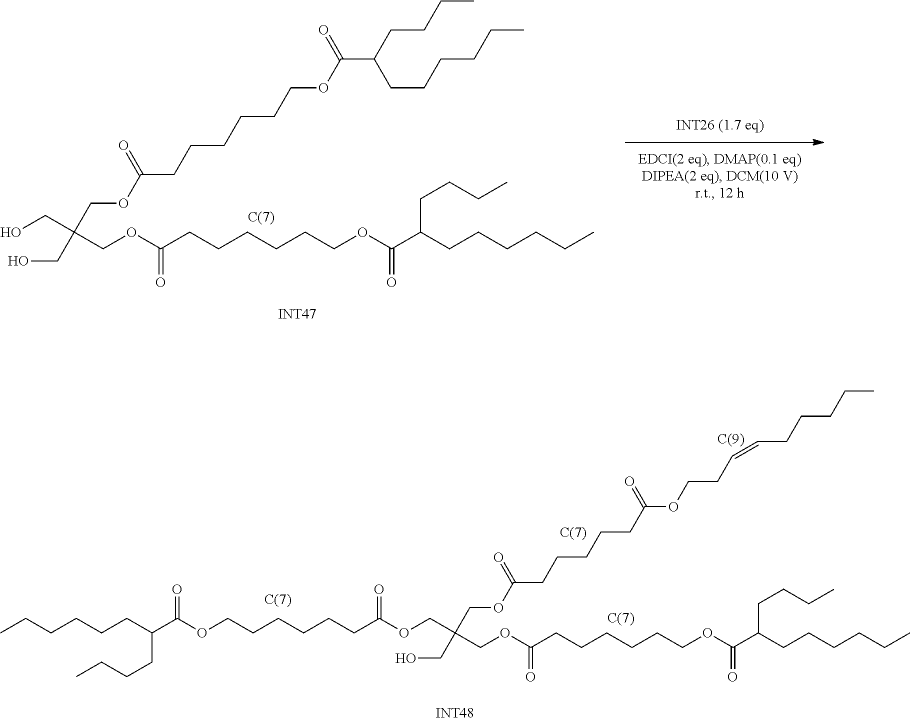

To a solution of [7-[2-[7-(2-butyloctanoyloxy)heptanoyloxymethyl]-3-hydroxy-2-(hydroxymethyl) propoxy]-7-oxo-heptyl]2-butyloctanoate (2 g, 2.64 mmol, 1 eq) in DCM (20 mL) was added EDCI (1.01 g, 5.28 mmol, 2 eq), 7-[(Z)-non-3-enoxy]-7-oxo-heptanoic acid (450.77 mg, 1.59 mmol, 0.6 eq), DMAP (32.27 mg, 264.17 mol, 0.1 eq) and DIPEA (682.84 mg, 5.28 mmol, 920.28 L, 2 eq). The mixture was stirred at 25° C. for 12 hr under N₂. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. Compound O7-[2,2-bis[7-(2-butyloctanoyloxy) heptanoyloxymethyl]-3-hydroxy-propyl]O1-[(Z)-non-3-enyl]heptanedioate (0.7 g, 683.95 mol, 25.89% yield) was obtained as a colorless oil.

### Step 4:

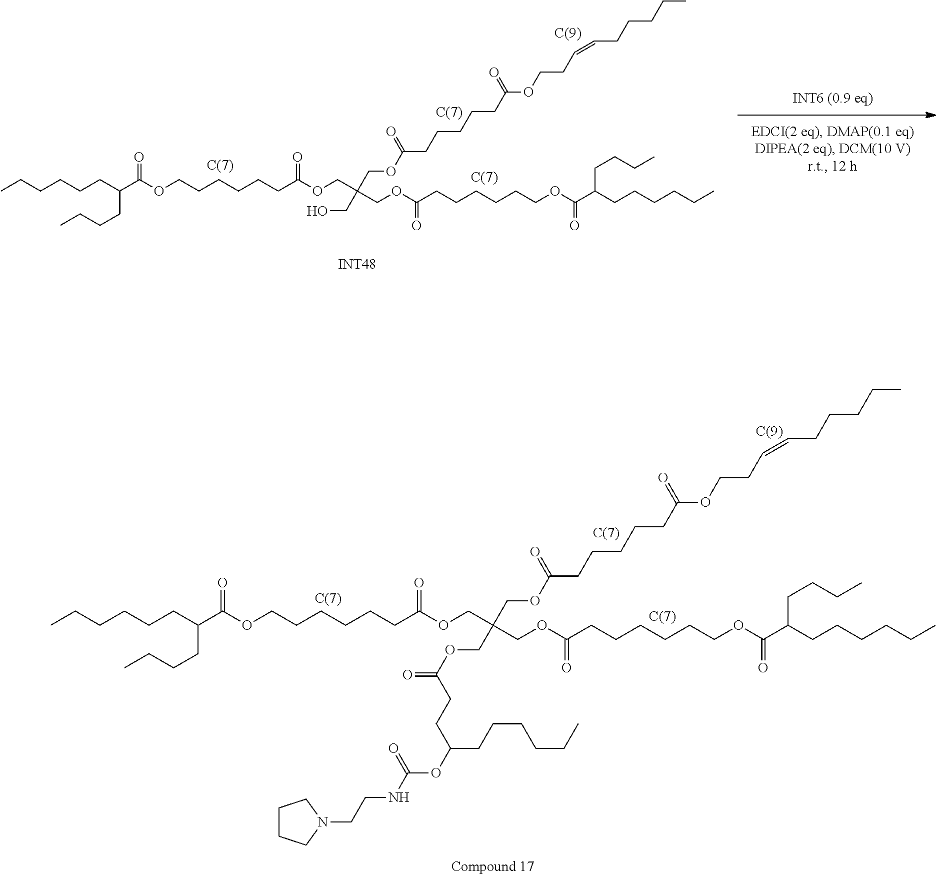

To a solution of O7-[2,2-bis[7-(2-butyloctanoyloxy)heptanoyloxymethyl]-3-hydroxy-propyl]O1-[(Z)-non-3-enyl]heptanedioate (0.7 g, 683.95 μmol, 1.2 eq) in DCM (7 mL) was added EDCI (218.53 mg, 1.14 mmol, 2 eq), 4-(2-pyrrolidin-5-ylethyl carbamoyloxy)decanoic acid (187.20 mg, 569.96 mol, 1 eq), DMAP (6.96 mg, 57.00 mol, 0.1 eq) and DIPEA (147.33 mg, 1.14 mmol, 198.55 μL, 2 eq). The mixture was stirred at 25° C. for 12 hr under N₂. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. Compound O7-[2,2-bis[7-(2-butyloctanoyloxy)heptanoyloxymethyl]-3-[4-(2-pyrrolidin-1-ylethylcarbamoyloxy)decanoyloxy]propyl]O1-[(Z)-non-3-enyl]heptanedioate (111 mg) was obtained as a colorless oil. ¹H NMR (400 MHz, CDCl₃) δ ppm 5.52-5.48 (m, 1H), 5.43-5.04 (m, 2H), 4.75 (s, 1H), 4.11 (s, 8H), 4.08-4.05 (t, J=6.8 Hz, 6H), 3.34 (s, 2H), 2.67-2.60 (m, 6H), 2.40-2.28 (m, 14H), 2.07-2.01 (q, J=6.8 Hz, J=13.6 Hz, 2H), 1.90-1.76 (m, 6H), 1.63-1.56 (m, 16H), 1.47-1.24 (m, 54H), 0.91-0.86 (m, 18H)

### Example 25—Compound 18

### [7-[2,2-bis[7-(2-butyloctanoyloxy)heptanoyloxymethyl]-3-[4-(2-pyrrolidin-1-ylethylcarbamoyloxy)decanoyloxy]propoxy]-7-oxo-heptyl]2-butyloctanoate

### Step 1:

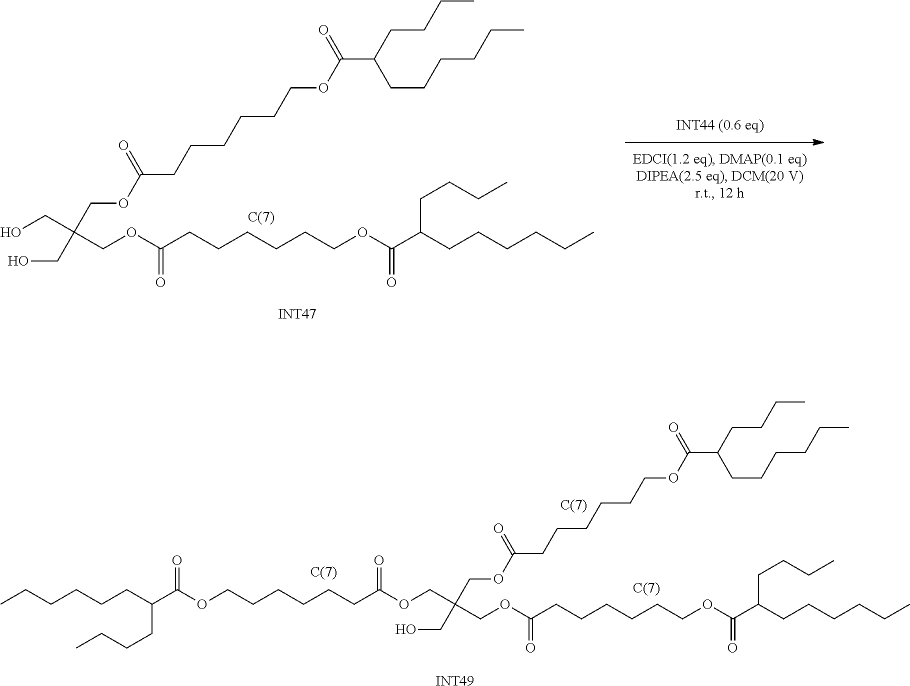

To a solution of [7-[2-[7-(2-butyloctanoyloxy)heptanoyloxymethyl]-3-hydroxyl-2-(hydroxymethyl) propoxy]-7-oxo-heptyl]2-butyloctanoate (3 g, 3.96 mmol, 1 eq) in DCM (30 mL) was added EDCI (911.56 mg, 4.76 mmol, 1.2 eq), DIPEA (51.21 mg, 396.26 mol, 69.02 μL, 0.1 eq), 7-(2-butyloctanoyloxy)heptanoic acid (780.99 mg, 2.38 mmol, 0.6 eq) and DMAP (1.21 g, 9.91 mmol, 2.5 eq). The mixture was stirred at 25° C. for 12 hr under N₂. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. Compound [7-[2,2-bis[7-(2-butyloctanoyloxy) heptanoyloxymethyl]-3-hydroxy-propoxy]-7-oxo-heptyl]2-butyloctanoate (1.5 g, 1.41 mmol, 35.46% yield) was obtained as a colorless oil. ¹H NMR (400 MHz, CDCl₃) δ ppm 4.12 (s, 6H), 4.09-4.06 (t, J=6.8 Hz, 6H), 3.515-3.50 (d, J=6 Hz), 2.59-2.56 (t, J=6.4 Hz, 1H), 2.36-2.28 (m, 9H), 1.67-1.55 (m, 24H), 1.45-1.26 (m, 48H), 0.91-0.87 (m, 18H)

### Step 2:

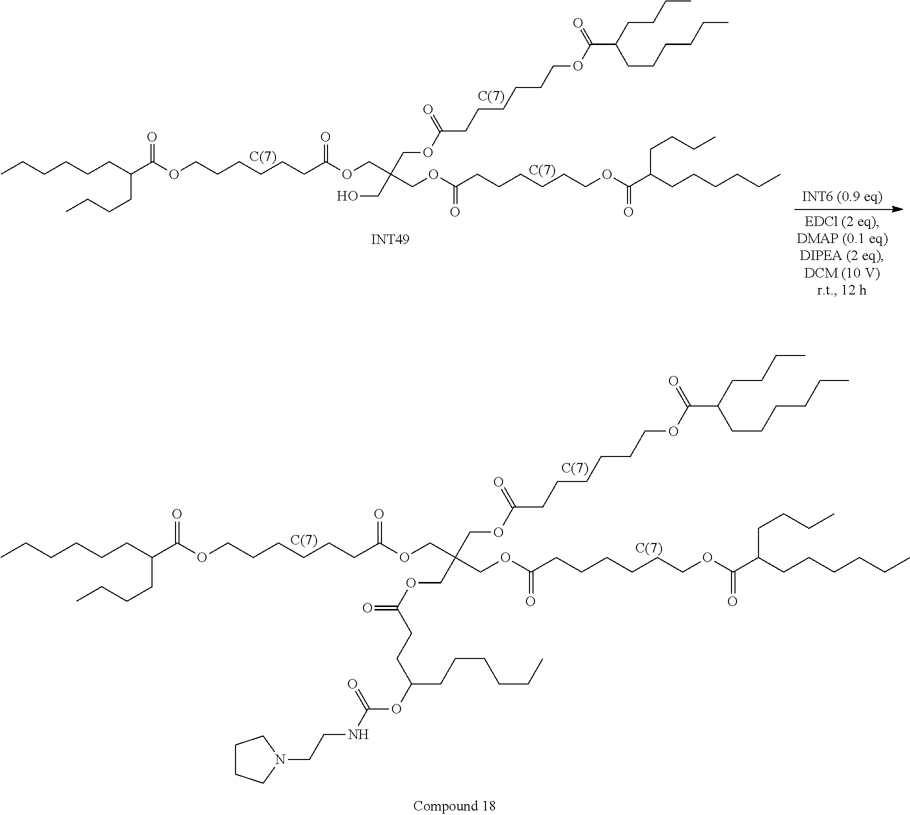

To a solution of [7-[2,2-bis[7-(2-butyloctanoyloxy)heptanoyloxymethyl]-3-hydroxy-propoxy]-7-oxo-heptyl]2-butyloctanoate (1.3 g, 1.22 mmol, 1 eq) in DCM (13 mL) was added EDCI (466.88 mg, 2.44 mmol, 2 eq), 4-(2-pyrrolidin-1-ylethylcarbamoyloxy)decanoic acid (359.96 mg, 1.10 mmol, 0.9 eq), DMAP (14.88 mg, 121.77 mol, 0.1 eq) and DIPEA (314.77 mg, 2.44 mmol, 424.21 L, 2 eq). The mixture was stirred at 25° C. for 12 hr under N₂. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. Compound [7-[2,2-bis[7-(2-butyloctanoyloxy)heptanoyloxymethyl]-3-[4-(2-pyrrolidin-1-ylethylcarbamoyloxy) decanoyloxy]propoxy]-7-oxo-heptyl]2-butyloctanoate (106 mg, 76.92 mol, 6.32% yield) was obtained as a colorless oil. ¹H NMR (400 MHz, CDCl₃) δ ppm 0.86-0.90 (td, J=6.94, 2.2 Hz, 21H) 1.23-1.33 (m, 42H) 1.34-1.51 (m, 20H) 1.51-1.69 (m, 20H) 1.78 (m, 6H) 1.84-1.96 (m, 1H) 2.30-2.42 (m, 10H) 2.53 (m, 4H) 2.60 (m, 2H) 3.29 (d, J=4.8 Hz, 2H) 4.07 (t, J=6.6 Hz, 6H) 4.12 (s, 8H) 4.75 (br s, 1H) 5.18 (br s, 1H).

### Example 26—Compound 19

### [7-[2-[7-(2-butyloctanoyloxy)heptanoyloxymethyl]-2-(4,4-dioctoxybutanoyloxymethyl)-3-[4-(2-pyrrolidin-1-ylethylcarbamoyloxy)decanoyloxy]propoxy]-7-oxo-heptyl]2-butyloctanoate

### Step 1:

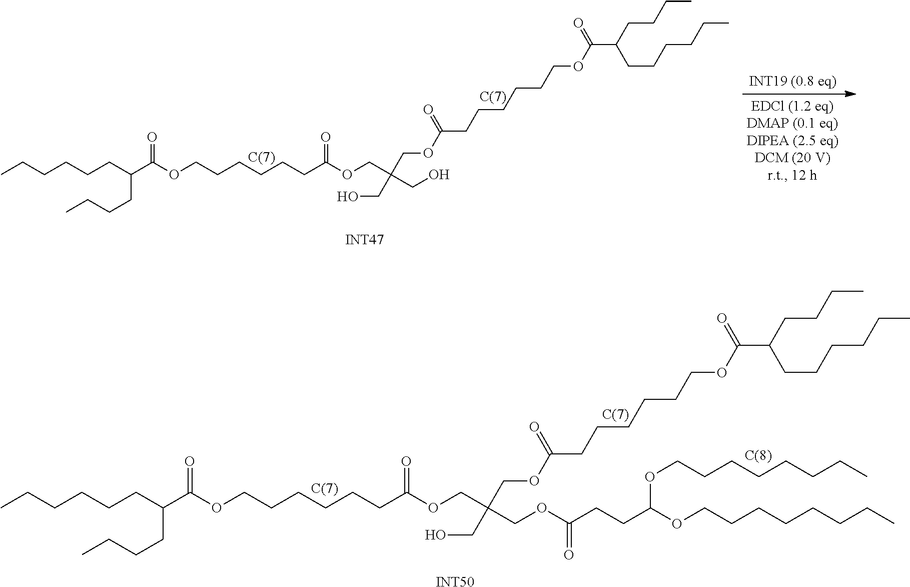

To a solution of [7-[2-[7-(2-butyloctanoyloxy)heptanoyloxymethyl]-3-hydroxy-2-(hydroxymethyl)propoxy]-7-oxoheptyl]2-butyloctanoate (2 g, 2.64 mmol, 1 eq) in DCM (20 mL) was added EDCI (607.70 mg, 3.17 mmol, 1.2 eq), DIPEA (853.56 mg, 6.60 mmol, 1.15 mL, 2.5 eq) and DMAP (32.27 mg, 264.17 mol, 0.1 eq), then a solution of 4,4-dioctoxybutanoic acid (728.11 mg, 2.11 mmol, 0.8 eq) in DCM (20 mL) was added to the above reaction mixture dropwise. The mixture was stirred at 20° C. for 12 hr under N₂. The reaction mixture was diluted with H₂O (20 mL) and extracted with DCM 60 mL (20 mL*3). The combined organic layers were concentrated under reduced pressure to give a residue. The residue was purified by column chromatography (SiO₂, Petroleum ether/Ethyl acetate=50/1 to 5/1). Compound [7-[2-[7-(2-butyloctanoyloxy)heptanoyloxymethyl]-2-(4,4-dioctoxybutanoyloxymethyl)-3-hydroxypropoxy]-7-oxo-heptyl]2-butyl octanoate (1.4 g, 1.29 mmol, 48.91% yield, 100% purity) was obtained as a colorless oil. ¹H NMR (400 MHz, CHLOROFORM-d) δ ppm 0.70-0.90 (m, 18H) 1.12-1.43 (m, 58H) 1.48-1.62 (m, 14H) 1.79-1.92 (m, 2H) 2.16-2.29 (m, 6H) 2.34 (t, J=7.6 Hz, 2H) 2.54 (t, J=7.0 Hz, 1H) 3.33 (dt, J=9.2, 6.8 Hz, 2H) 3.40-3.57 (m, 4H) 3.99 (t, J=6.6 Hz, 4H) 4.04 (s, 6H) 4.41 (t, J=5.4 Hz, 1H).

### Step 2:

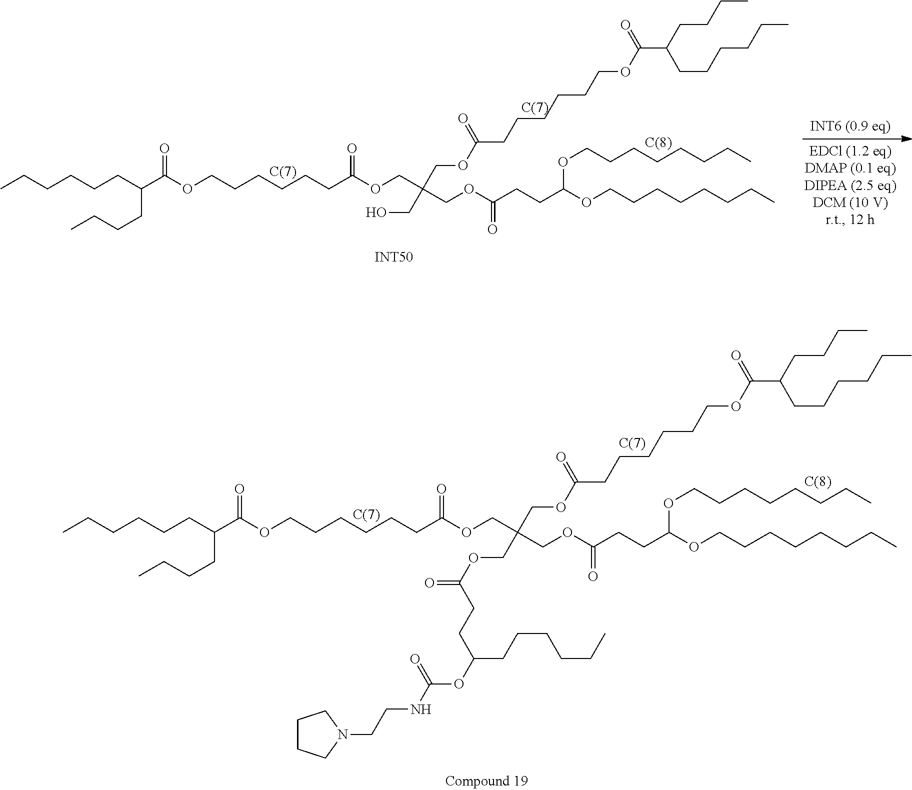

To a solution of [7-[2-[7-(2-butyloctanoyloxy)heptanoyloxymethyl]-2-(4,4-dioctoxybutanoyloxymethyl)-3-hydroxypropoxy]-7-oxo-heptyl]2-butyloctanoate (1 g, 922.85 mol, 1 eq) in DCM (10 mL) was added EDCI (212.29 mg, 1.11 mmol, 1.2 eq), DIPEA (298.18 mg, 2.31 mmol, 401.86 L, 2.5 eq) and DMAP (11.27 mg, 92.29 mol, 0.1 eq), then 4-(2-pyrrolidin-1-ylethylcarbamoyloxy)decanoic acid (272.80 mg, 830.57 mol, 0.9 eq) was added to the above reaction mixture. The mixture was stirred at 20° C. for 12 hr under N₂. The reaction mixture was diluted with H₂O (20 mL) and extracted with DCM 30 mL (10 mL*3). The combined organic layers were concentrated under reduced pressure to give a residue. The residue was purified by column chromatography (SiO₂, Petroleum ether/Ethyl acetate=50/1 to 1/1). Compound [7-[2-[7-(2-butyloctanoyloxy)heptanoyloxymethyl]-2-(4,4-dioctoxybutanoyloxymethyl)-3-[4-(2-pyrrolidin-1-ylethylcarbamoyloxy)decanoyloxy]propoxy]-7-oxo-heptyl]2-butyloctanoate (0.102 g, 73.17 mol, 7.93% yield) was obtained as a colorless oil. ¹H NMR (400 MHz, CDCl₃) δ ppm 0.86-0.90 (td, J=7.0, 2.4 Hz, 6H) 1.22-1.32 (m, 12H) 1.36-1.45 (m, 12H) 1.59-1.67 (m, 6H) 2.35-2.38 (m, 4H) 3.50 (s, 2H) 3.70-3.76 (m, 4H) 4.05-4.09 (t, J=6.6 Hz, 2H) 4.25 (s, 2H).

### Example 27—Compound 20

### [7-[2,2-bis(4,4-dioctoxybutanoyloxymethyl)-3-[4-(2-pyrrolidin-1-ylethylcarbamoyloxy) decanoyloxy]propoxy]-7-oxo-heptyl]2-butyloctanoate

### Step 1:

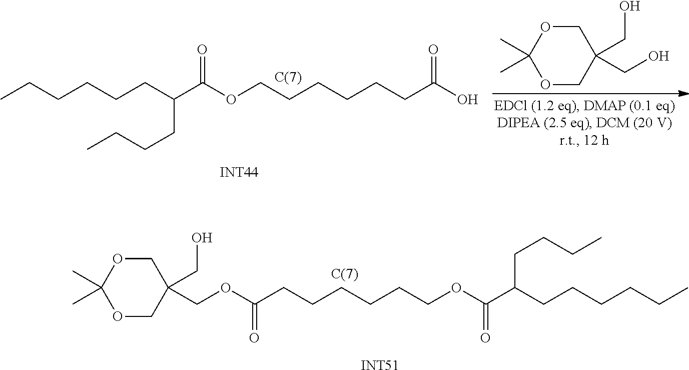

To a solution of 7-(2-butyloctanoyloxy)heptanoic acid (5.83 g, 17.73 mmol, 1 eq) in DCM (58 mL) was added EDCI (4.08 g, 21.28 mmol, 1.2 eq), [5-(hydroxymethyl)-2,2-dimethyl-1,3-dioxan-5-yl]methanol (5 g, 28.38 mmol, 1.6 eq), DIPEA (5.73 g, 44.34 mmol, 7.72 mL, 2.5 eq) and DMAP (216.66 mg, 1.77 mmol, 0.1 eq). The mixture was stirred at 20° C. for 12 hr under N₂. The reaction mixture was diluted with H₂O (100 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound [7-[[5-(hydroxymethyl)-2,2-dimethyl-1,3-dioxan-5-yl]methoxy]-7-oxo-heptyl]2-butyloctanoate (3 g, 6.16 mmol, 34.76% yield) was obtained as a colorless oil. ¹H NMR (400 MHz, CDCl₃) δ ppm 0.86-0.90 (td, J=7.0, 2.4 Hz, 6H) 1.22-1.32 (m, 12H) 1.36-1.45 (m, 12H) 1.59-1.67 (m, 6H) 2.35-2.38 (m, 4H) 3.50 (s, 2H) 3.70-3.76 (m, 4H) 4.05-4.09 (t, J=6.6 Hz, 2H) 4.25 (s, 2H).

### Step 2:

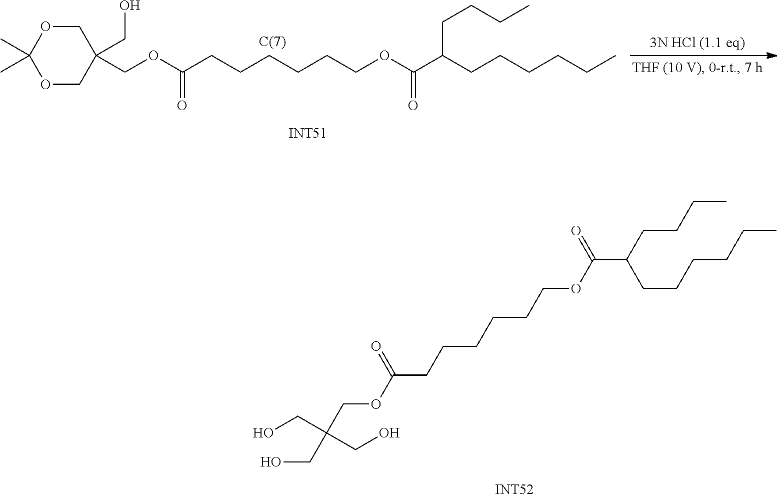

To a solution of [7-[[5-(hydroxymethyl)-2,2-dimethyl-1,3-dioxan-5-yl]methoxy]-7-oxo-heptyl]2-butyloctanoate (3 g, 6.16 mmol, 1 eq) in THF (30 mL) was added HCl (3 M, 2.26 mL, 1.1 eq) slowly at 0° C. The mixture was stirred at 20° C. for 7 hr under N₂. The reaction mixture was added to sat.NaHCO₃ to adjust to pH=9 and extracted with EtOAc 90 mL (30 mL*3). The combined organic layers were concentrated under reduced pressure to give a residue. The residue was purified by column chromatography (SiO₂, Petroleum ether/Ethyl acetate=100/1 to 0/1). Compound [7-[3-hydroxy-2,2-bis(hydroxymethyl) propoxy]-7-oxo-heptyl]2-butyloctanoate (1.5 g, 3.36 mmol, 54.49% yield) was obtained as a colorless oil. ¹H NMR (400 MHz, CDCl₃) δ ppm 0.86-0.90 (td, J=6.94, 2.6 Hz, 6H) 1.21-1.47 (m, 19H) 1.63-1.66 (m, 6H) 2.32 (s, 1H) 2.36-2.39 (t, J=7.4 Hz, 2H) 2.53-2.65 (m, 2H) 3.66 (s, 6H) 4.06-4.09 (t, J=6.6 Hz, 2H) 4.23 (s, 2H).

### Step 3:

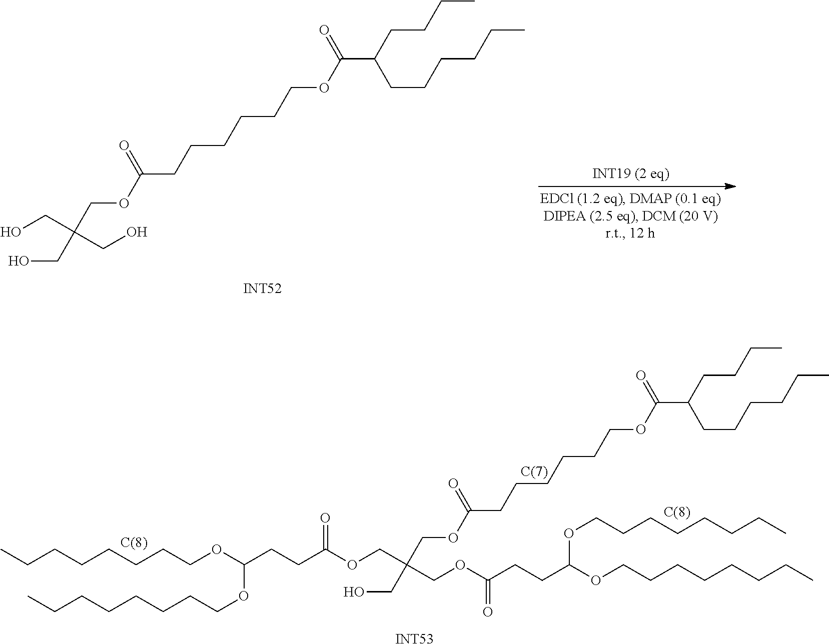

To a solution of [7-[3-hydroxy-2,2-bis(hydroxymethyl) propoxy]-7-oxo-heptyl]2-butyloctanoate (1.5 g, 3.36 mmol, 1 eq) in DCM (30 mL) was added EDCI (772.62 mg, 4.03 mmol, 1.2 eq), 4,4-dioctoxybutanoic acid (2.31 g, 6.72 mmol, 2 eq), DIPEA (1.09 g, 8.40 mmol, 1.46 mL, 2.5 eq) and DMAP (41.03 mg, 335.86 mol, 0.1 eq). The mixture was stirred at 20° C. for 12 hr under N₂. The reaction mixture was concentrated under reduced pressure to remove solvent. The residue was diluted with H₂O (50 mL) and extracted with DCM 150 mL (50 mL*3). The combined organic layers were concentrated under reduced pressure to give a residue. The residue was purified by column chromatography (SiO₂, Petroleum ether/Ethyl acetate=100/1 to 0/1). Compound [7-[2,2-bis(4,4-dioctoxybutanoyloxymethyl)-3-hydroxy-propoxy]-7-oxo-heptyl]2-butyloctanoate (1.2 g, 1.09 mmol, 32.49% yield) was obtained as a colorless oil. ¹H NMR (400 MHz, CDCl₃) δ ppm 0.87-0.91 (m, 18H) 1.28-1.38 (m, 56H) 1.53-1.65 (m, 16H) 1.91-1.96 (m, 4H) 2.29-2.35 (m, 3H) 2.40-2.44 (t, J=7.4 Hz, 4H) 2.64-2.67 (t, J=7.0 Hz, 1H) 3.38-3.44 (dt, J=9.2, 6.8 Hz, 4H) 3.51-3.59 (m, 6H) 4.05-4.09 (t, J=6.6 Hz, 2H) 4.11 (s, 6H) 4.47-4.50 (t, J=5.4 Hz, 2H).

### Step 4:

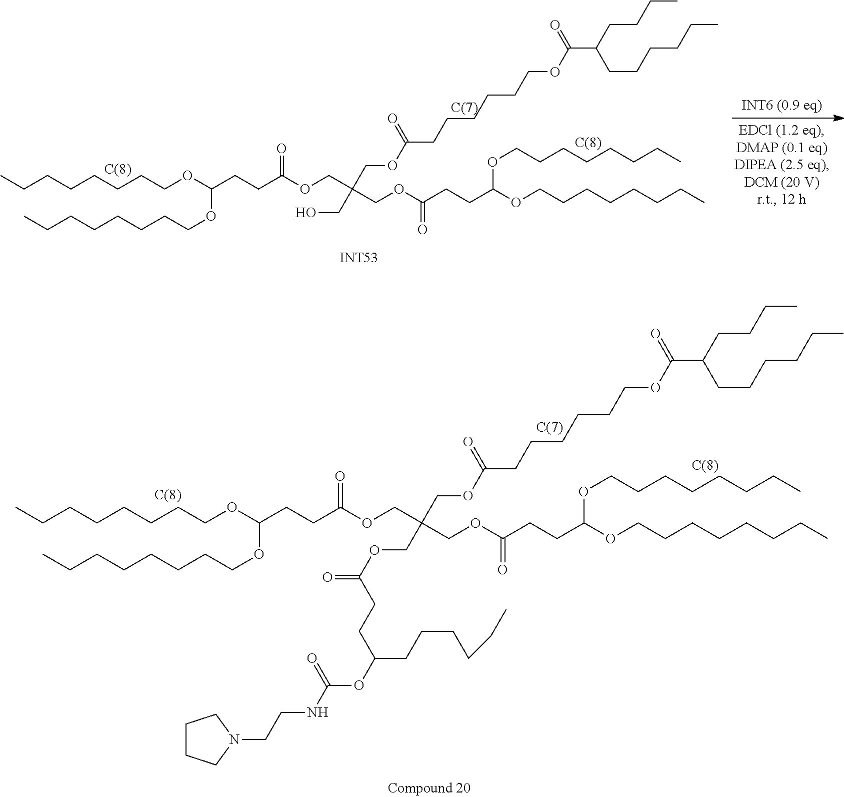

To a solution of [7-[2,2-bis(4,4-dioctoxybutanoyloxymethyl)-3-hydroxy-propoxy]-7-oxo-heptyl]2-butyl octanoate (1.2 g, 1.09 mmol, 1 eq) in DCM (24 mL) was added EDCI (251.04 mg, 1.31 mmol, 1.2 eq), 4-(2-pyrrolidin-1-ylethylcarbamoyloxy)decanoic acid (322.58 mg, 982.14 mol, 0.9 eq), DIPEA (141.04 mg, 1.09 mmol, 190.08 L, 2.5 eq) and DMAP (13.33 mg, 109.13 mol, 0.1 eq). The mixture was stirred at 20° C. for 12 hr under N₂. The reaction mixture was diluted with H₂O (50 mL) and extracted with DCM 150 mL (50 mL*3). The combined organic layers were concentrated under reduced pressure to give a residue. The residue was purified by column chromatography (SiO₂, Petroleum ether/Ethyl acetate=100/1 to 0/1). Compound [7-[2,2-bis(4,4-dioctoxybutanoyloxymethyl)-3-[4-(2-pyrrolidin-1-ylethylcarbamoyloxy) decanoyloxy]propoxy]-7-oxo-heptyl]2-butyloctanoate (0.12 g, 85.10 mol, 7.80% yield) was obtained as a colorless oil. ¹H NMR (400 MHz, CDCl₃) δ ppm 0.81-0.94 (m, 21H) 1.24-1.51 (m, 66H) 1.52-1.66 (m, 18H) 1.79 (m, 4H) 1.89-1.95 (m, 4H) 2.25-2.43 (m, 9H) 2.53 (m, 4H) 2.61 (m, 2H) 3.20-3.34 (m, 2H) 3.41 (dt, J=9.2, 6.8 Hz, 4H) 3.56 (dt, J=9.2, 6.8 Hz, 4H) 4.07 (t, J=6.6 Hz, 2H) 4.12 (s, 8H) 4.48 (t, J=5.4 Hz, 2H) 4.68-4.90 (m, 1H) 5.20 (br s, 1H).

### Example 28—Compound 21

### O9-[2,2-bis[[9-[(Z)-oct-3-enoxy]-9-oxo-nonanoyl]oxymethyl]-3-[4-(2-pyrrolidin-1ylethylcarbamoyloxy)decanoyloxy]propyl]O1-[(Z)-oct-3-enyl]nonanedioate

### Step 1:

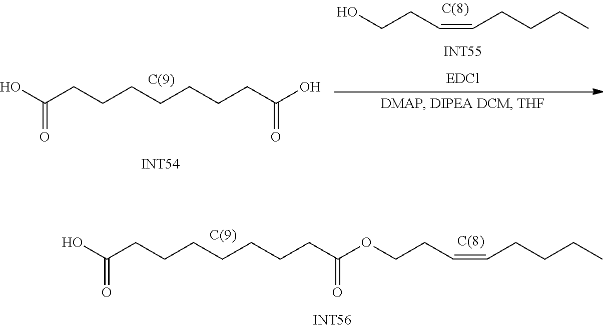

To a solution of (Z)-oct-3-en-1-ol (20 g, 155.99 mmol, 1 eq) in DCM (100 mL) and THF (100 mL) was added EDCI (35.88 g, 187.19 mmol, 1.2 eq), nonanedioic acid (146.80 g, 779.96 mmol, 5 eq), DIPEA (50.40 g, 389.98 mmol, 67.93 mL, 2.5 eq) and DMAP (1.91 g, 15.60 mmol, 0.1 eq) under N₂ atmosphere. The reaction mixture was stirred at 25° C. for 12 hours under N₂ atmosphere. The reaction mixture was diluted with H₂O (100 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound 9-[(Z)-oct-3-enoxy]-9-oxo-nonanoic acid (20 g, 67.02 mmol, 42.96% yield) was obtained as a colorless oil.

### Step 2:

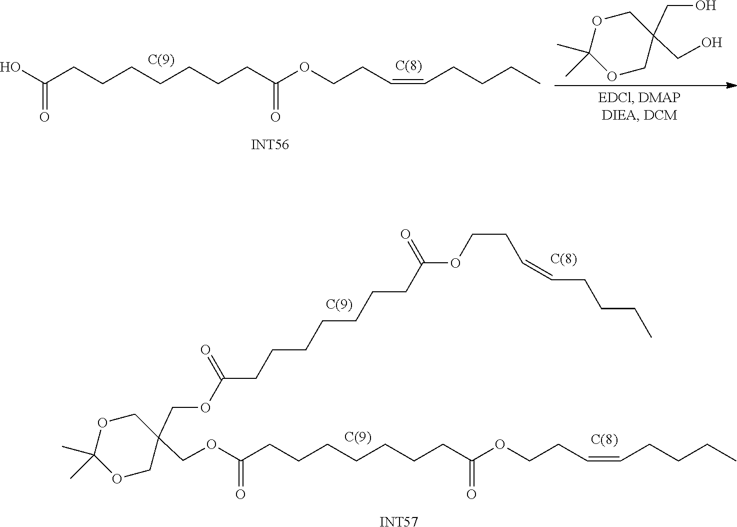

To a solution of 9-[(Z)-oct-3-enoxy]-9-oxo-nonanoic acid (11.29 g, 37.83 mmol, 1 eq) in DCM (110 mL) was added EDCI (8.70 g, 45.40 mmol, 1.2 eq), [5-(hydroxymethyl)-2,2-dimethyl-1,3-dioxan-5-yl]methanol (3 g, 17.03 mmol, 0.45 eq), DIPEA (12.22 g, 94.58 mmol, 16.47 mL, 2.5 eq) and DMAP (462.21 mg, 3.78 mmol, 0.1 eq) under N₂ atmosphere. The reaction mixture was stirred at 25° C. for 12 hours under N₂ atmosphere. The reaction mixture was diluted with H₂O (100 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound O9-[[2,2-dimethyl-5-[[9-[(Z)-oct-3-enoxy]-9-oxo-nonanoyl]oxymethyl]-1,3-dioxan-5-yl]methyl]O1-[(Z)-oct-3-enyl]nonanedioate (7 g, 9.50 mmol, 25.10% yield) was obtained as a colorless oil. ¹H NMR (400 MHz, CDCl₃) δ ppm 5.52-5.49 (m, 2H), 5.35-5.32 (m, 2H), 4.10 (s, 4H), 4.06 (t, J=12.0 Hz, 8H), 3.74 (s, 4H), 2.40-2.35 (m, 4H), 2.33-2.27 (m, 4H), 2.05-2.02 (m, 4H), 1.60 (t, J=8.0 Hz, 9H), 1.42 (s, 6H), 1.34-1.31 (m, 20H), 0.92-0.88 (m, 6H).

### Step 3:

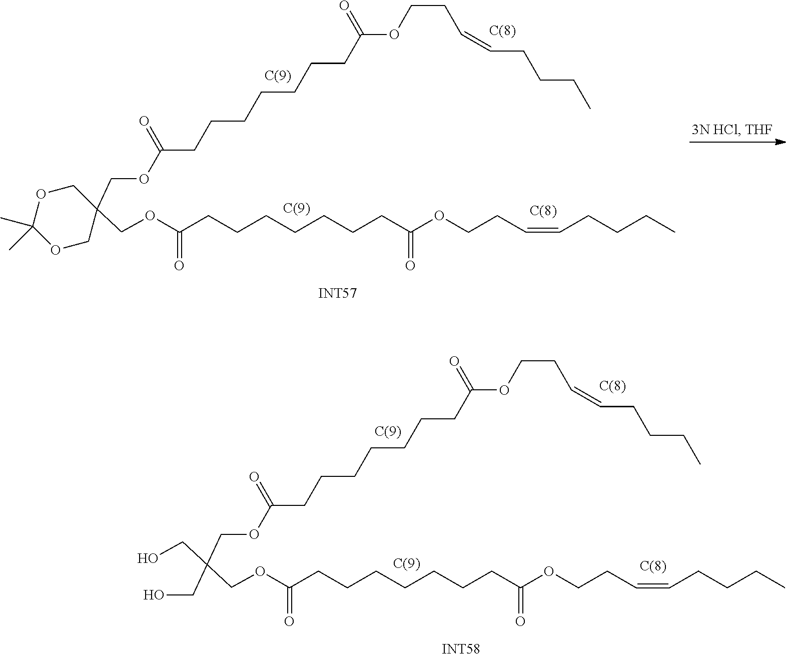

To a solution of O9-[[2,2-dimethyl-5-[[9-[(Z)-oct-3-enoxy]-9-oxo-nonanoyl]oxymethyl]-1,3-dioxan-5-yl]methyl]O1-[(Z)-oct-3-enyl]nonanedioate (7 g, 9.50 mmol, 1 eq) in THF (70 mL) was added HCl (3 M, 3.48 mL, 1.1 eq) at 0° C. under N₂ atmosphere. The reaction mixture was stirred at 20° C. for 2 hours under N₂ atmosphere. The reaction mixture was diluted with aq. NaHCO₃ (70 mL) and extracted with DCM 300 mL (100 mL*3). The combined organic layers were concentrated under reduced pressure to give a residue. The residue was purified by column chromatography (SiO₂, Petroleum ether/Ethyl acetate=100/1 to 0/1). Compound O9-[2,2-bis(hydroxymethyl)-3-[9-[(Z)-oct-3-enoxy]-9-oxo-nonanoyl]oxy-propyl]O1-[(Z)-oct-3-enyl]nonanedioate (2.5 g, 3.59 mmol, 37.77% yield) was obtained as a colorless oil.

### Step 4:

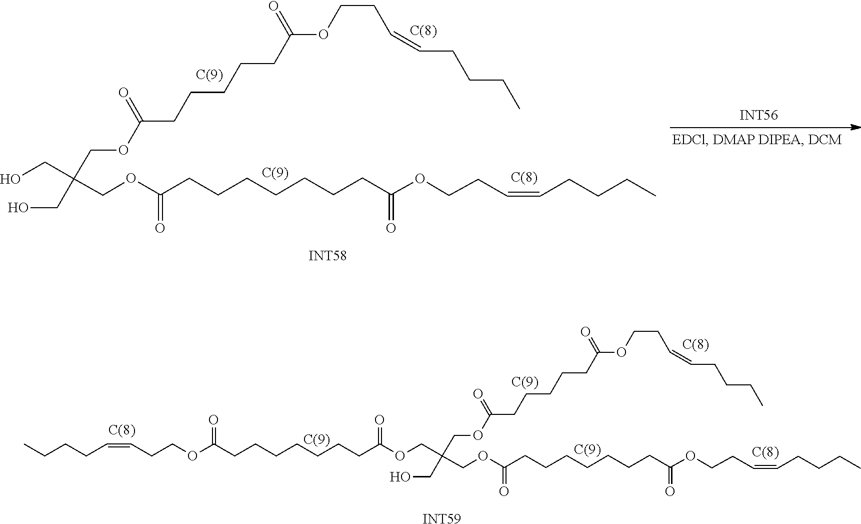

To a solution of O9-[2,2-bis(hydroxymethyl)-3-[9-[(Z)-oct-3-enoxy]-9-oxo-nonanoyl]oxy-propyl]O1-[(Z)-oct-3-enyl]nonanedioate (1 g, 1.43 mmol, 1 eq) in DCM (10 mL) was added EDCI (330.07 mg, 1.72 mmol, 1.2 eq), 9-[(Z)-oct-3-enoxy]-9-oxo-nonanoic acid (256.91 mg, 860.90 mol, 0.6 eq), DIPEA (463.60 mg, 3.59 mmol, 624.80 L, 2.5 eq) and DMAP (17.53 mg, 143.48 mol, 0.1 eq) under N₂ atmosphere. The reaction mixture was stirred at 20° C. for 12 hr under N₂ atmosphere. The reaction mixture was diluted with H₂O (10 mL) and extracted with DCM 30 mL (10 mL*3). The combined organic layers were concentrated under reduced pressure to give a residue. The residue was purified by column chromatography (SiO₂, Petroleum ether/Ethyl acetate=100/1 to 0/1). Compound 09-[2-(hydroxymethyl)-3-[9-[(Z)-oct-3-enoxy]-9-oxo-nonanoyl]oxy-2-[[9-[(Z)-oct-3-enoxy]-9-oxo-nonanoyl]oxymethyl]propyl]O1-[(Z)-oct-3-enyl]nonanedioate (0.5 g, 511.59 mol, 35.66% yield) was obtained as a colorless oil. ¹H NMR (400 MHz, CDCl₃) δ ppm 5.52-5.49 (m, 3H), 5.36-5.33 (m, 3H), 4.11 (s, 6H), 4.06 (t, J=4.0 Hz, 6H), 3.51 (d, J=4.0 Hz, 2H), 2.60 (t, J=4.0 Hz, 1H), 2.40-2.27 (m, 18H), 2.07-2.02 (m, 6H), 1.34-1.32 (m, 28H), 0.92-0.89 (m, 9H).

### Step 5:

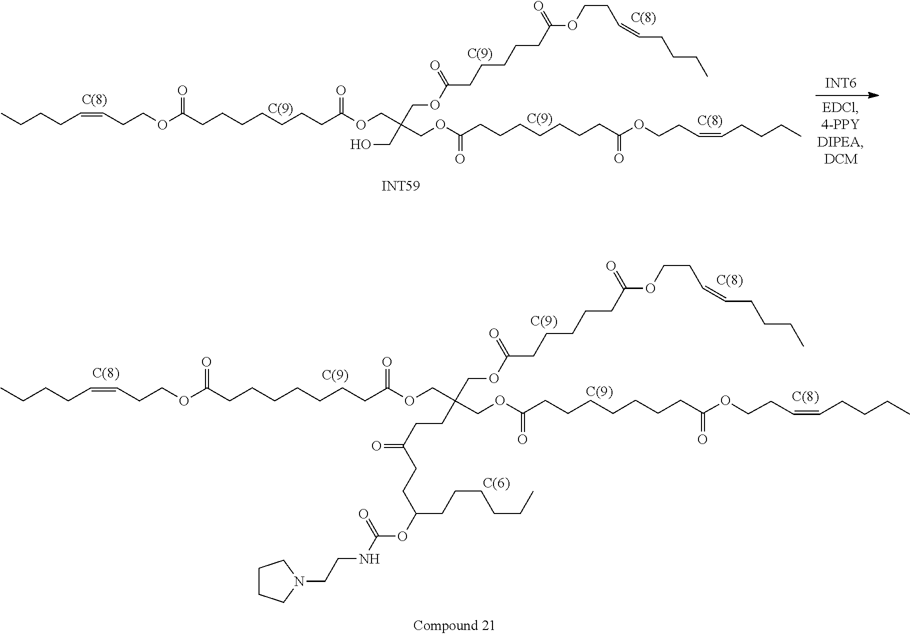

To a solution of O9-[2-(hydroxymethyl)-3-[9-[(Z)-oct-3-enoxy]-9-oxo-nonanoyl]oxy-2-[[9-[(Z)-oct-3-enoxy]-9-oxo-nonanoyl]oxymethyl]propyl]O1-[(Z)-oct-3-enyl]nonanedioate (0.5 g, 511.59 mol, 1 eq) in DCM (5 mL) was added EDCI (117.69 mg, 613.91 mol, 1.2 eq), 4-(2-pyrrolidin-1-ylethylcarbamoyloxy)decanoic acid (201.63 mg, 613.91 mol, 1.2 eq), DIPEA (165.30 mg, 1.28 mmol, 222.77 L, 2.5 eq) and 4-pyrrolidin-1-ylpyridine (7.58 mg, 51.16 mol, 0.1 eq) under N₂ atmosphere. The reaction mixture was stirred at 20° C. for 12 h under N₂ atmosphere. The reaction mixture was concentrated under reduced pressure to remove solvent. The residue was diluted with H₂O (10 mL) and extracted with DCM 30 mL (10 mL*3). The combined organic layers were concentrated under reduced pressure to give a residue. Compound O9-[2,2-bis[[9-[(Z)-oct-3-enoxy]-9-oxo-nonanoyl]oxymethyl]-3-[4-(2-pyrrolidin-1ylethylcarbamoyloxy)decanoyloxy]propyl]O1-[(Z)-oct-3-enyl]nonanedioate (133 mg) was obtained as a colorless oil. ¹H NMR (400 MHz, CDCl₃) δ ppm 5.52-5.49 (m, 3H), 5.36-5.33 (m, 3H), 5.26-5.25 (m, 1H), 4.75 (s, 1H), 4.12 (d, J=3.2 Hz, 8H), 4.06 (t, J=7.2 Hz, 6H), 3.32 (d, J=4.4 Hz, 2H), 2.61 (d, J=25.6 Hz, 6H), 2.40-2.35 (m, 8H), 2.32-2.27 (m, 12H), 2.08-2.03 (m, 6H), 1.81 (s, 6H), 1.63-1.49 (m, 16H), 1.36-1.27 (m, 36H), 0.92-0.86 (m, 12H).

### Example 29—Compound 22

### O9-[2,2-bis[[9-[(Z)-non-3-enoxy]-9-oxo-nonanoyl]oxymethyl]-3-[4-(2-pyrrolidin-1ylethylcarbamoyloxy)decanoyloxy]propyl]O1-[(Z)-non-3-enyl]nonanedioate

### Step 1:

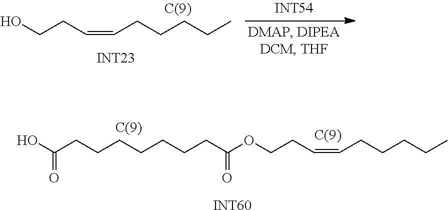

To a solution of (Z)-non-3-en-1-ol (20 g, 140.61 mmol, 1 eq) in DCM (100 mL) and THF (100 mL) was added EDCI (32.35 g, 168.73 mmol, 1.2 eq), nonanedioic acid (132.33 g, 703.05 mmol, 5 eq), DIPEA (45.43 g, 351.52 mmol, 61.23 mL, 2.5 eq) and DMAP (1.72 g, 14.06 mmol, 0.1 eq) under N₂ atmosphere. The mixture was stirred at 20° C. for 12 hr under N₂ atmosphere. The reaction mixture was concentrated under reduced pressure to remove solvent. The residue was diluted with H₂O (300 mL) and extracted with DCM 900 mL (300 mL*3). The combined organic layers were concentrated under reduced pressure to give a residue. The residue was purified by column chromatography (SiO₂, Petroleum ether/Ethyl acetate=100/1 to 0/1). Compound 9-[(Z)-non-3-enoxy]-9-oxo-nonanoic acid (20 g, 64.01 mmol, 45.52% yield) was obtained as a colorless oil. ¹H NMR (400 MHz, CDCl₃) δ ppm 11.21 (br s, 1H), 5.56-5.45 (m, 1H), 5.39-5.29 (m, 1H), 4.07 (t, J=6.8 Hz, 2H), 2.42-2.27 (m, 6H), 2.08-1.99 (m, 2H), 1.71-1.56 (m, 4H), 1.38-1.20 (m, 12H), 0.89 (t, J=6.8 Hz, 3H).

### Step 2:

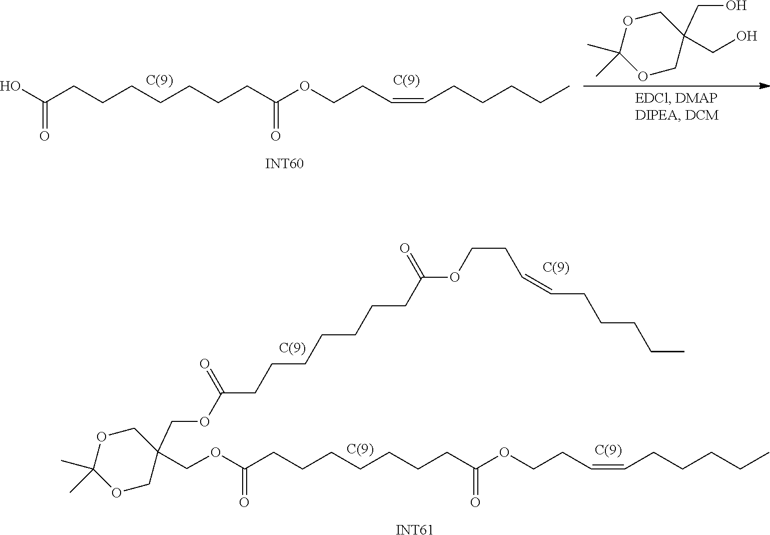

To a solution of 9-[(Z)-non-3-enoxy]-9-oxo-nonanoic acid (3.94 g, 12.61 mmol, 1 eq) in DCM (40 mL) was added EDCI (2.90 g, 15.13 mmol, 1.2 eq), [5-(hydroxymethyl)-2,2-dimethyl-1,3-dioxan-5-yl]methanol (1 g, 5.68 mmol, 0.45 eq), DIPEA (4.07 g, 31.53 mmol, 5.49 mL, 2.5 eq) and DMAP (154.07 mg, 1.26 mmol, 0.1 eq) under N₂ atmosphere. The mixture was stirred at 20° C. for 12 hr under N₂ atmosphere. The reaction mixture was diluted with H₂O (50 mL) and extracted with DCM 90 mL (30 mL*3). The combined organic layers were concentrated under reduced pressure to give a residue. The residue was purified by column chromatography (SiO₂, Petroleum ether/Ethyl acetate=100/1 to 0/1). Compound O9-[[2,2-dimethyl-5-[[9-[(Z)-non-3-enoxy]-9-oxo-nonanoyl]oxymethyl]-1,3-dioxan-5-yl]methyl]O1-[(Z)-non-3-enyl]nonanedioate (2 g, 2.61 mmol, 20.73% yield) was obtained as a colorless oil. ¹H NMR (400 MHz, CDCl₃) δ ppm 5.51-5.49 (m, 2H), 5.35-5.32 (m, 2H), 4.10 (s, 4H), 4.06 (t, J=12.0 Hz, 8H), 3.74 (s, 4H), 2.40-2.35 (m, 4H), 2.33-2.27 (m, 4H), 2.04 (t, J=8.0 Hz, 4H), 1.6 (t, J=8.0 Hz, 9H), 1.42 (s, 6H), 1.34-1.31 (m, 20H), 0.92-0.88 (m, 6H).

### Step 3:

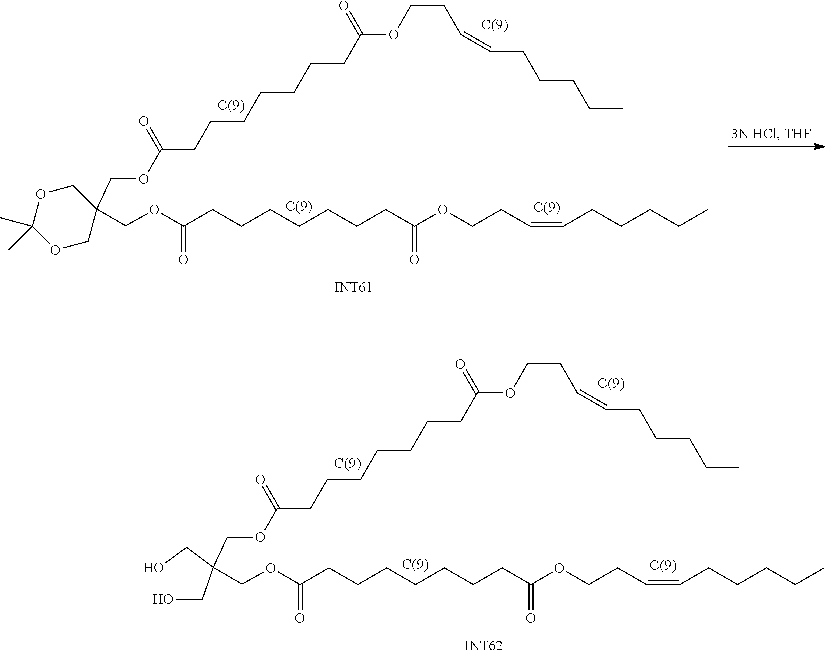

To a solution of O9-[[2,2-dimethyl-5-[[9-[(Z)-non-3-enoxy]-9-oxo-nonanoyl]oxymethyl]-1,3-dioxan-5-yl]methyl]O1-[(Z)-non-3-enyl]nonanedioate (6.5 g, 8.50 mmol, 1 eq) in THF (65 mL) was added HCl (3 M, 3.12 mL, 1.1 eq) at 0° C. under N₂ atmosphere. The reaction mixture was stirred at 20° C. for 12 hours under N₂ atmosphere. The reaction mixture was diluted with sat. NaHCO₃ (100 mL) and extracted with EtOAc 300 mL (100 mL*3). The combined organic layers were concentrated under reduced pressure to give a residue. The residue was purified by column chromatography (SiO₂, Petroleum ether/Ethyl acetate=100/1 to 0/1). Compound O9-[2,2-bis(hydroxymethyl)-3-[9-[(Z)-non-3-enoxy]-9-oxo-nonanoyl]oxy-propyl]O1-[(Z)-non-3-enyl]nonanedioate (2.5 g, 3.45 mmol, 40.59% yield) was obtained as a colorless oil.

### Step 4:

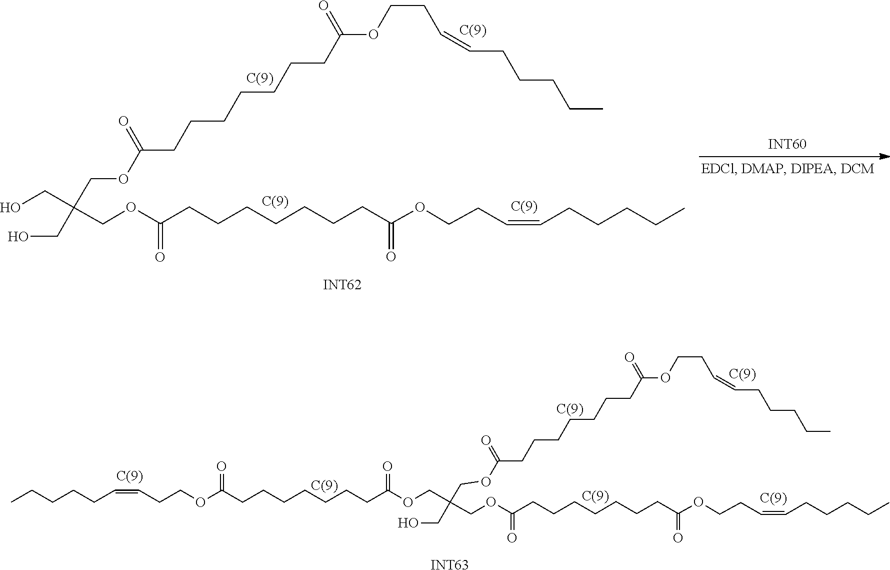

To a solution of O9-[2,2-bis(hydroxymethyl)-3-[9-[(Z)-non-3-enoxy]-9-oxo-nonanoyl]oxy-propyl]O1-[(Z)-non-3-enyl]nonanedioate (0.6 g, 827.58 mol, 1 eq) in DCM (6 mL) was added EDCI (190.38 mg, 993.10 mol, 1.2 eq), 9-[(Z)-non-3-enoxy]-9-oxo-nonanoic acid (155.14 mg, 496.55 mol, 0.6 eq), DIPEA (267.40 mg, 2.07 mmol, 360.38 L, 2.5 eq) and DMAP (10.11 mg, 82.76 mol, 0.1 eq) under N₂ atmosphere. The reaction mixture was stirred at 20° C. for 12 h under N₂ atmosphere. The reaction mixture was diluted with H₂O (30 mL) and extracted with DCM 90 mL (30 mL*3). The combined organic layers were concentrated under reduced pressure to give a residue. The residue was purified by column chromatography (SiO₂, Petroleum ether/Ethyl acetate=100/1 to 0/1). Compound 09-[2-(hydroxymethyl)-3-[9-[(Z)-non-3-enoxy]-9-oxo-nonanoyl]oxy-2-[[9-[(Z)-non-3-enoxy]-9-oxononanoyl]oxymethyl]propyl]O1-[(Z)-non-3-enyl]nonanedioate (0.3 g, 294.28 mol, 35.56% yield) was obtained as a colorless oil.

### Step 5:

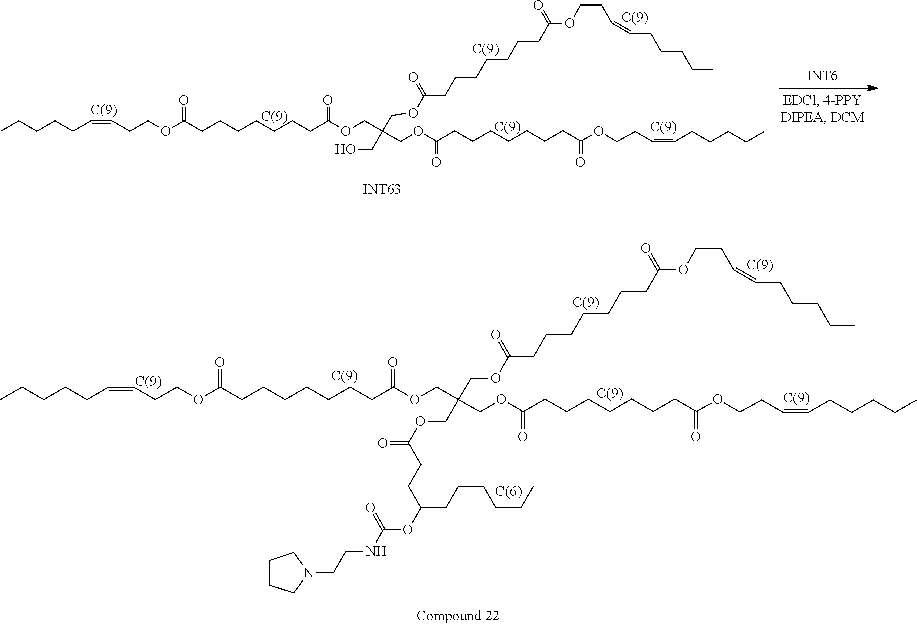

To a solution of O9-[2-(hydroxymethyl)-3-[9-[(Z)-non-3-enoxy]-9-oxo-nonanoyl]oxy-2-[[9-[(Z)-non-3-enoxy]-9-oxo-nonanoyl]oxymethyl]propyl]O1-[(Z)-non-3-enyl]nonanedioate (1.5 g, 1.47 mmol, 1 eq) in DCM (20 mL) was added EDCI (338.49 mg, 1.77 mmol, 1.2 eq), DIPEA (475.42 mg, 3.68 mmol, 640.73 μL, 2.5 eq), 4-(2-pyrrolidin-1-ylethylcarbamoyloxy)decanoic acid (579.93 mg, 1.77 mmol, 1.2 eq) and 4-pyrrolidin-1-ylpyridine (21.81 mg, 147.14 μmol, 0.1 eq) under N₂ atmosphere. The reaction mixture was stirred at 20° C. for 12 h under N₂ atmosphere. The reaction mixture was concentrated under reduced pressure to remove solvent. The residue was diluted with H₂O (30 mL) and extracted with DCM 90 mL (30 mL*3). The combined organic layers were concentrated under reduced pressure to give a residue. The residue was purified by column chromatography (SiO₂, Petroleum ether/Ethyl acetate=100/1 to 0/1). Compound O9-[2,2-bis[[9-[(Z)-non-3-enoxy]-9-oxo-nonanoyl]oxymethyl]-3-[4-(2-pyrrolidin-1ylethylcarbamoyloxy)decanoyloxy]propyl]O1-[(Z)-non-3-enyl]nonanedioate (0.1 g, 75.20 μmol, 5.11% yield) was obtained as a colorless oil. ¹H NMR (400 MHz, CDCl₃) δ ppm 5.52-5.48 (m, 3H), 5.36-5.33 (m, 3H), 4.76 (br s, 1H), 4.11 (s, 8H), 4.07 (t, J=7.0 Hz, 6H), 3.34 (br s, 2H), 2.75-2.55 (m, 4H), 2.38 (m, 8H), 2.30 (m, 12H), 2.04 (m, 6H), 1.90-1.73 (m, 6H), 1.70-1.58 (m, 16H), 1.38-1.27 (m, 44H), 0.91-0.85 (m, 12H).

### Example 30—Compound 23

### O8-[2,2-bis[(8-nonoxy-8-oxo-octanoy)oxymethyl]-3-[4-(2-pyrrolidin-1-ylethylcarbamoyloxy)decanoyloxy]propyl]O1-nonyl octanedioate

### Step 1:

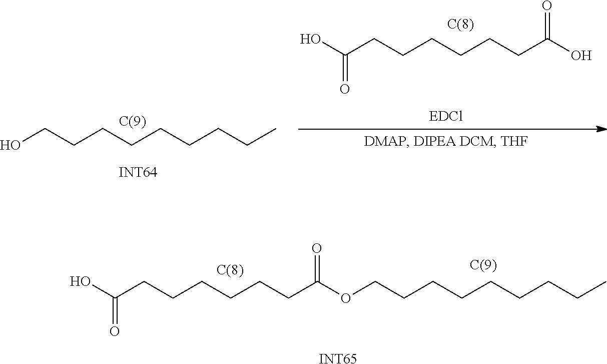

To a solution of nonan-1-ol (20 g, 138.64 mmol, 1 eq) in DCM (600 mL) and THF (600 mL) was added octanedioic acid (120.76 g, 693.22 mmol, 5 eq), DIPEA (53.76 g, 415.93 mmol, 72.45 mL, 3 eq), EDCI (34.55 g, 180.24 mmol, 1.3 eq) and DMAP (3.39 g, 27.73 mmol, 0.2 eq) under N₂ atmosphere. The reaction mixture was stirred at 20° C. for 12 hr under N₂ atmosphere. The reaction mixture was poured into H₂O (1000 mL) and extract ed with DCM (300 mL×3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=5/1 to 3/1). Compound 8-nonoxy-8-oxo-octanoic acid (33 g, 109.84 mmol, 79.23% yield) was obtained as a colorless oil. ¹H NMR (400 MHz, CDCl₃) δ ppm 11.40 (br s, 1H), 4.06 (t, J=6.8 Hz, 2H), 2.43-2.20 (m, 4H), 1.73-1.55 (m, 6H), 1.43-1.15 (m, 16H), 0.96-0.81 (m, 3H).

### Step 2:

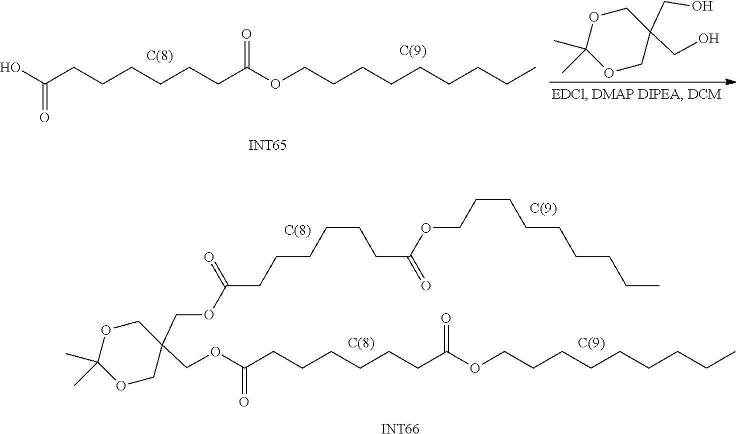

To a solution of 8-nonoxy-8-oxo-octanoic acid (18.94 g, 63.06 mmol, 1 eq) and [5-(hydroxymethyl)-2,2-dimethyl-1,3-dioxan-5-yl]methanol (5 g, 28.38 mmol, 0.45 eq) in DCM (200 mL) was added DIPEA (20.37 g, 157.64 mmol, 27.46 mL, 2.5 eq), EDCI (14.51 g, 75.67 mmol, 1.2 eq) and DMAP (770.34 mg, 6.31 mmol, 0.1 eq) under N₂ atmosphere. The reaction mixture was stirred at 20° C. for 12 hr under N₂ atmosphere. The reaction mixture was poured into H₂O (50 mL) and extracted with DCM (50 mL×3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=20/1 to 10/1). Compound O8-[[2,2-dimethyl-5-[(8-nonoxy-8-oxo-octanoyl)oxymethyl]-1,3-dioxan-5-yl]methyl]O1-nonyl octanedioate (17 g, 22.94 mmol, 72.65% yield) was obtained as a colorless oil. ¹H NMR (400 MHz, CDCl₃) δ ppm 4.11 (s, 4H), 4.06 (t, J=6.8 Hz, 4H), 3.74 (s, 4H), 2.31 (m, 8H), 1.70-1.55 (m, 12H), 1.42 (s, 6H), 1.39-1.09 (m, 32H), 0.94-0.80 (m, 6H).

### Step 3:

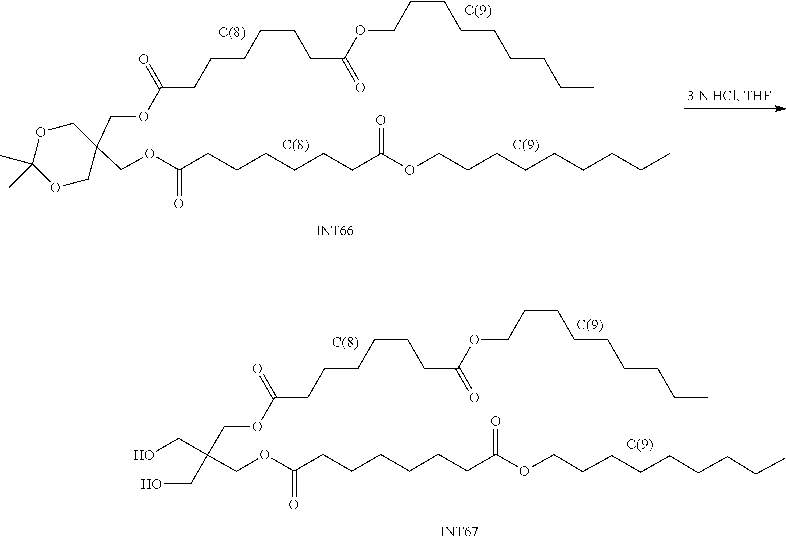

To a solution of O8-[[2,2-dimethyl-5-[(8-nonoxy-8-oxo-octanoyl)oxymethyl]-1,3-dioxan-5-yl]methyl]O1-nonyl octanedioate (17 g, 22.94 mmol, 1 eq) in THF (170 mL) was added HCl (3 M, 9.18 mL, 1.2 eq) at 0° C. under N₂. The reaction mixture was stirred at 20° C. for 12 hr under N₂ atmosphere. The reaction mixture was diluted with sat. NaHCO₃ (100 mL) and extracted with EtOAc 300 mL (100 mL*3). The combined organic layers were concentrated under reduced pressure to give a residue. The residue was purified by column chromatography (SiO₂, Petroleum ether/Ethyl acetate=5/1 to 3/1). Compound O8-[2,2-bis(hydroxymethyl)-3-(8-nonoxy-8-oxo-octanoyl)oxy-propyl]O1-nonyl octanedioate (8.7 g, 12.41 mmol, 54.10% yield) was obtained as a colorless oil. ¹H NMR (400 MHz, CDCl₃) δ ppm 4.13 (s, 4H), 4.05 (t, J=6.8 Hz, 4H), 3.58 (br s, 4H), 2.86 (br s, 2H), 2.40-2.16 (m, 8H), 1.71-1.50 (m, 12H), 1.41-1.13 (m, 32H), 0.87 (t, J=6.8 Hz, 6H).

### Step 4:

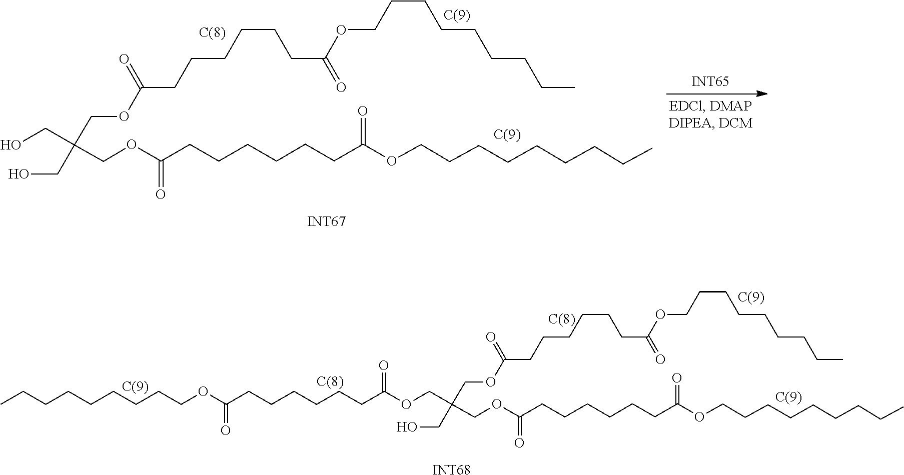

To a solution of 8-nonoxy-8-oxo-octanoic acid (2.24 g, 7.45 mmol, 0.6 eq) and O8-[2,2bis(hydroxymethyl)-3-(8-nonoxy-8-oxo-octanoyl)oxy-propyl]O1-nonyl octanedioate (8.7 g, 12.41 mmol, 1 eq) in DCM (87 mL) was added DIPEA (4.01 g, 31.03 mmol, 5.40 mL, 2.5 eq), EDCI (2.86 g, 14.89 mmol, 1.2 eq) and DMAP (151.62 mg, 1.24 mmol, 0.1 eq). The reaction mixture was stirred at 20° C. for 12 hr under N₂ atmosphere. The reaction mixture was poured into H₂O (100 mL) and extracted with DCM (50 mL×3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=8/1 to 5/1). Compound O8-[2-(hydroxymethyl)-3-(8-nonoxy-8-oxo-octanoyl)oxy-2-[(8-nonoxy-8-oxooctanoyl)oxymethyl]propyl]O1-nonyl octanedioate (3.4 g, 3.46 mmol, 27.85% yield) was obtained as a colorless oil. ¹H NMR (400 MHz, CDCl₃) δ ppm 4.10 (s, 6H), 4.04 (t, J=6.8 Hz, 6H), 3.49 (br d, J=6.2 Hz, 2H), 2.65 (br t, J=6.6 Hz, 1H), 2.30 (m, 12H), 1.52-1.68 (m, 18H), 1.11-1.39 (m, 48H), 0.77-0.90 (m, 9H),

### Step 5:

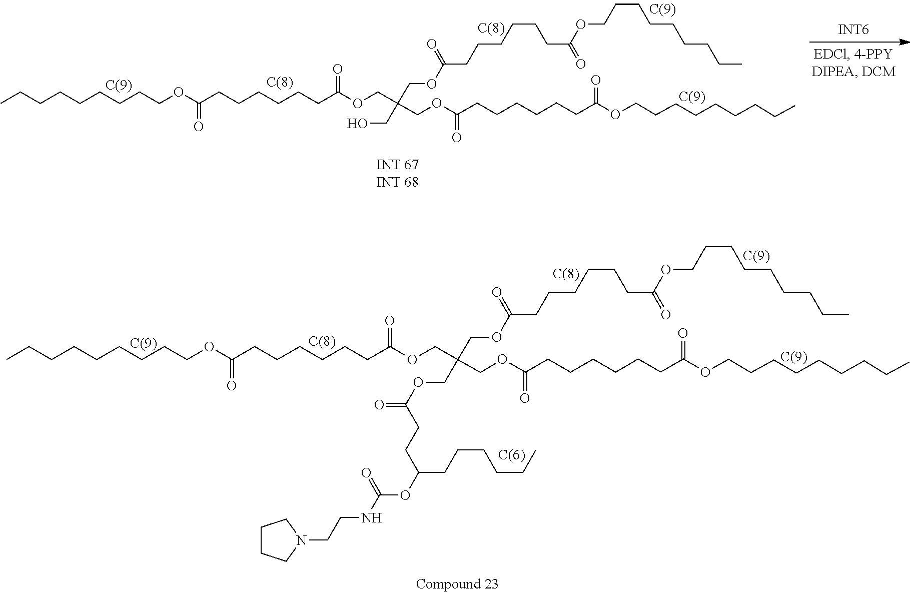

To a solution of O8-[2-(hydroxymethyl)-3-(8-nonoxy-8-oxo-octanoyl)oxy-2-[(8-nonoxy-8-oxo-octanoyl)-oxymethyl]propyl]O1-nonyl octanedioate (800 mg, 813.51 mol, 1 eq) and 4-(2-pyrrolidin-1-ylethylcarbamoyloxy)decanoic acid (534.39 mg, 1.63 mmol, 2 eq) in DCM (8 mL) was added DIPEA (262.85 mg, 2.03 mmol, 354.25 μL, 2.5 eq), EDCI (187.14 mg, 976.21 mol, 1.2 eq) and 4-pyrrolidin-1-ylpyridine (12.06 mg, 81.35 mol, 0.1 eq) under N₂ atmosphere. The reaction mixture was stirred at 20° C. for 12 hr under N₂ atmosphere. The reaction mixture was poured into H₂O (20 mL) and extracted with DCM (10 mL×3). The combined organic layers were washed with brine (20 mL×2), dried over Na₂SO₄, filtered and the filtrate was concentrated under reduced pressure to give a residue. The residue was purified by column chromatography (SiO₂, dichloromethane/methanol=50/1 to 5/1). Compound O8-[2,2-bis[(8-nonoxy-8-oxo-octanoy)oxymethyl]-3-[4-(2-pyrrolidin-1-ylethylcarbamoyloxy)decanoyloxy]propyl]O1-nonyl octanedioate (110 mg, 81.64 mol, 10.06% yield, 96.02% purity) was obtained as a colorless oil. ¹H NMR (400 MHz, CDCl₃) δ ppm 5.19 (br s, 1H), 4.75 (br s, 1H), 4.11 (s, 8H), 4.06 (t, J=6.8 Hz, 6H), 3.29 (br d, J=5.4 Hz, 2H), 3.29 (br d, J=5.4 Hz, 2H), 2.59 (br t, J=5.8 Hz, 2H), 2.52 (br s, 4H), 2.42-2.34 (m, 2H), 2.33-2.19 (m, 12H), 1.89 (br d, J=6.8 Hz, 1H), 1.78 (br s, 5H), 1.70 (br s, 2H), 1.61 (br d, J=3.2 Hz, 18H), 1.43-1.22 (m, 56H), 0.92-0.84 (m, 12H).

### Example 31—Compound 24

### O7-[2,2-bis[(7-nonoxy-7-oxo-heptanoyl)oxymethyl]-3-[4-(2-pyrrolidin-1-ylethylcarbamoyloxy) decanoyloxy]propyl]O1-nonyl heptanedioate

### Step 1:

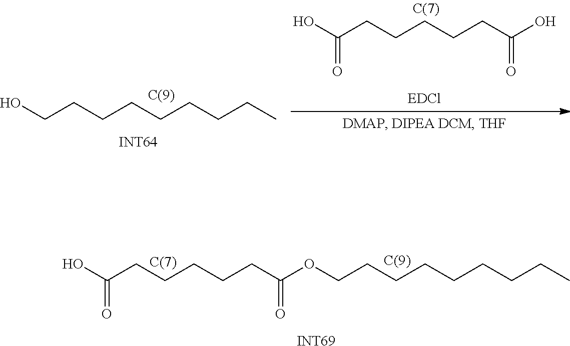

To a solution of nonan-1-ol (20 g, 138.64 mmol, 1 eq) in DCM (200 mL) and THF (200 mL) was added EDCI (34.55 g, 180.24 mmol, 1.3 eq), heptanedioic acid (111.03 g, 693.22 mmol, 5 eq), DMAP (1.69 g, 13.86 mmol, 0.1 eq) and DIPEA (44.80 g, 346.61 mmol, 60.37 mL, 2.5 eq) under N₂ atmosphere. The reaction mixture was stirred at 25° C. for 12 hr under N₂ atmosphere. The reaction mixture was diluted with H₂O (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound 7-nonoxy-7-oxo-heptanoic acid (30 g, 104.75 mmol, 75.55% yield) was obtained as a colorless oil. ¹H NMR (400 MHz, CDCl₃) δ ppm 11.78-10.36 (br s, 1H), 4.06 (t, J=6.8 Hz, 2H), 2.49-2.24 (m, 4H), 1.65 (m, 6H), 1.50-1.49 (m, 14H), 0.99-0.80 (m, 3H).

### Step 2:

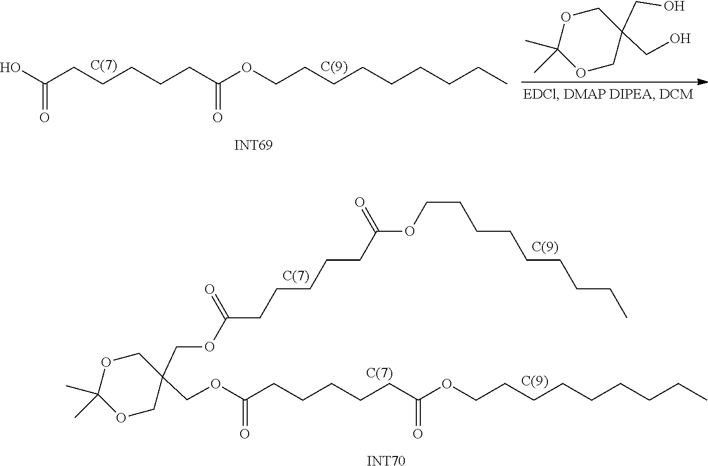

To a solution of 7-nonoxy-7-oxo-heptanoic acid (14.45 g, 50.44 mmol, 1 eq) in DCM (145 mL) was added EDCI (11.60 g, 60.53 mmol, 1.2 eq), [5-(hydroxymethyl)-2,2-dimethyl-1,3-dioxan-5-yl]methanol (4 g, 22.70 mmol, 0.45 eq), DMAP (616.27 mg, 5.04 mmol, 0.1 eq) and DIPEA (16.30 g, 126.11 mmol, 21.97 mL, 2.5 eq) under N₂ atmosphere. The reaction mixture was stirred at 25° C. for 12 hr under N₂ atmosphere. The reaction mixture was diluted with H₂O (300 mL) and extracted with DCM 800 mL (200 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O7-[[2,2-dimethyl-5-[(7-nonoxy-7-oxo-heptanoyl)oxymethyl]-1,3-dioxan-5-yl]methyl]O1-nonyl heptanedioate (7.5 g, 10.52 mmol, 20.85% yield) was obtained as a colorless oil. ¹H NMR (400 MHz, CDCl₃) δ ppm 4.11 (s, 4H), 4.06 (t, J=6.8 Hz, 4H), 3.74 (s, 4H), 2.32 (m, 8H), 1.72-1.55 (m, 12H), 1.42 (s, 6H), 1.40-1.09 (m, 28H), 0.88 (t, J=6.8 Hz, 6H).

### Step 3:

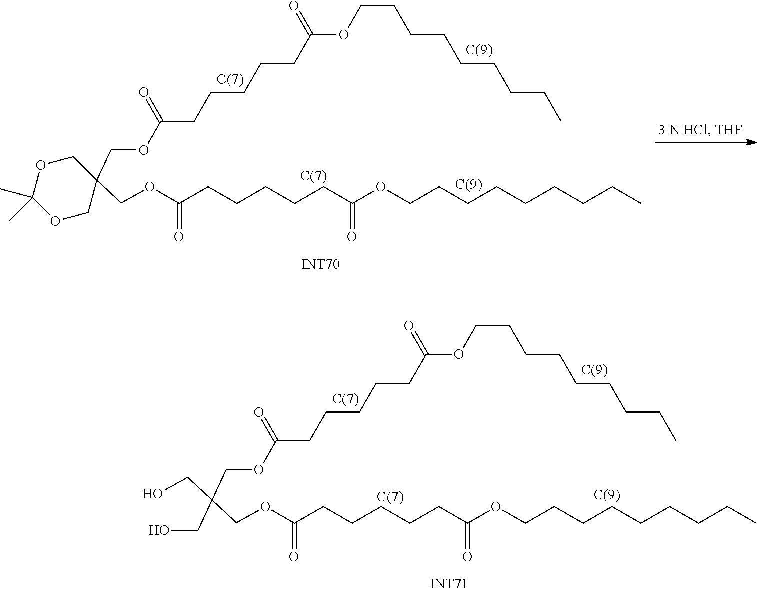

To a solution of O7-[[2,2-dimethyl-5-[(7-nonoxy-7-oxo-heptanoyl)oxymethyl]-1,3-dioxan-5-yl]methyl]O1-nonyl heptanedioate (7 g, 9.82 mmol, 1 eq) in THF (70 mL) was added HCl (3 M, 3.60 mL, 1.1 eq) at 0° C. under N₂ atmosphere. The reaction mixture was stirred at 25° C. for 7 hr under N₂ atmosphere. The reaction mixture was diluted with sat. NaHCO₃ (50 mL) and extracted with EtOAc 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2,2-bis(hydroxymethyl)-3-(7-nonoxy-7-oxo-heptanoyl)oxy-propyl]O1-nonyl heptanedioate (2.5 g, 3.72 mmol, 37.84% yield) was obtained as a colorless oil.

### Step 4:

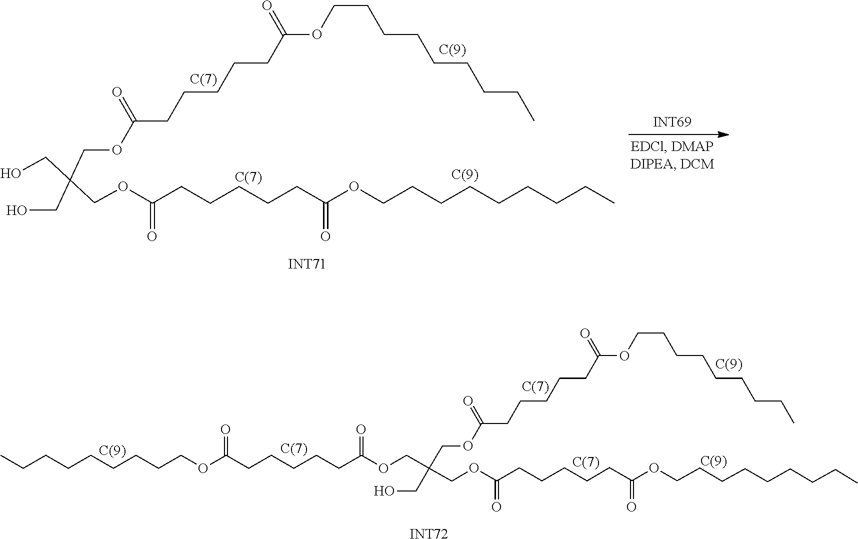

To a solution of O7-[2,2-bis(hydroxymethyl)-3-(7-nonoxy-7-oxo-heptanoyl)oxy-propyl]O1-nonylheptanedioate (2.5 g, 3.72 mmol, 1 eq) in DCM (25 mL) was added EDCI (854.63 mg, 4.46 mmol, 1.2 eq), 7-nonoxy-7-oxo-heptanoic acid (638.42 mg, 2.23 mmol, 0.6 eq), DMAP (45.39 mg, 371.51 mol, 0.1 eq) and DIPEA (1.20 g, 9.29 mmol, 1.62 mL, 2.5 eq) under N₂ atmosphere. The reaction mixture was stirred at 25° C. for 12 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2-(hydroxymethyl)-3-(7-nonoxy-7-oxo-heptanoyl)oxy-2-[(7-nonoxy-7-oxo-heptanoyl)oxymethyl]propyl]O1-nonyl heptanedioate (1.2 g, 1.27 mmol, 34.31% yield) was obtained as a colorless oil. ¹H NMR (400 MHz, CDCl₃) δ ppm 4.11 (s, 6H), 4.05 (t, J=13.6 Hz, 6H), 3.51 (d, J=6.4 Hz, 2H), 2.66 (t, J=6.8 Hz, 1H), 2.36-2.29 (m, 12H), 1.68-1.60 (m, 18H), 1.39-1.27 (m, 42H), 0.92-0.87 (m, 9H).

### Step 5:

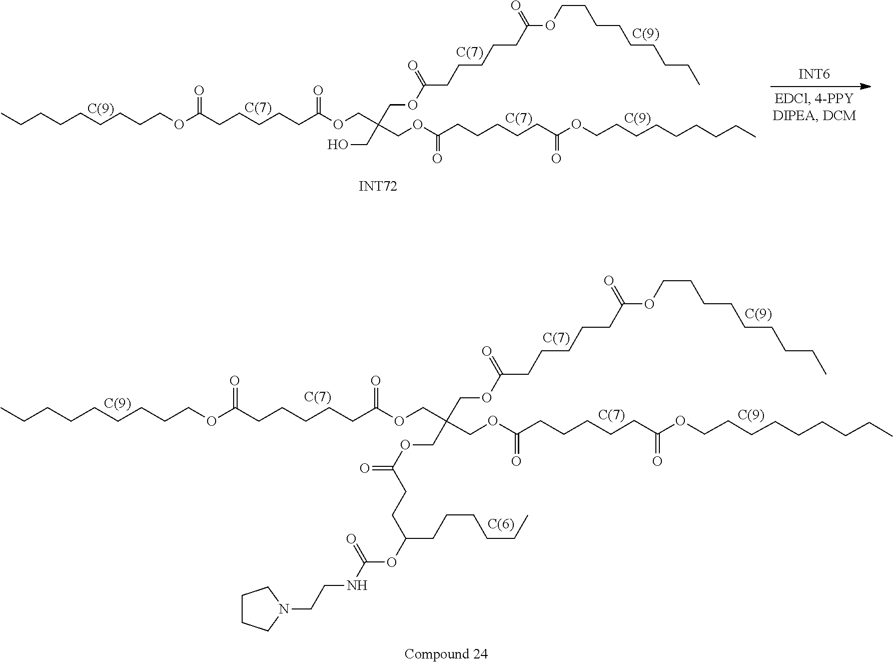

To a solution of O7-[2-(hydroxymethyl)-3-(7-nonoxy-7-oxo-heptanoyl)oxy-2-[(7-nonoxy-7-oxo-heptanoyl)oxymethyl]propyl]O1-nonyl heptanedioate (1.2 g, 1.27 mmol, 1 eq) in DCM (12 mL) was added EDCI (293.26 mg, 1.53 mmol, 1.2 eq), 4-(2-pyrrolidin-1-ylethylcarbamoyloxy)decanoic acid (251.22 mg, 764.89 mol, 0.6 eq), 4-pyrrolidin-1-ylpyridine (18.89 mg, 127.48 mol, 0.1 eq) and DIPEA (411.90 mg, 3.19 mmol, 555.12 μL, 2.5 eq) under N₂ atmosphere. The reaction mixture was stirred at 25° C. for 12 hr under N₂ atmosphere. The reaction mixture was diluted with H₂O (50 mL) and extracted with DCM 50 mL (25 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2,2-bis[(7-nonoxy-7-oxo-heptanoyl)oxymethyl]-3-[4-(2-pyrrolidin-1-ylethylcarbamoyloxy) decanoyloxy]propyl]O1-nonyl heptanedioate (120 mg, 91.78 mol, 7.20% yield, 95.74% purity) was obtained as a colorless oil. ¹H NMR (400 MHz, CDCl₃) δ ppm 5.20 (s, 1H), 4.76-4.75 (s, 1H), 4.11 (s, 8H), 4.06 (t, J=6.8 Hz, 6H), 3.30-3.29 (s, 2H), 2.57 (d, J=30.8 Hz, 6H), 2.40-2.29 (m, 14H), 1.91-1.79 (m, 6H), 1.68-1.56 (m, 22H), 1.52-1.45 (m, 2H), 1.39-1.28 (m, 46H), 0.93-0.87 (m, 12H).

### Example 32—Compound 25

### O8-[2,2-bis[[8-[(Z)-non-3-enoxy]-8-oxo-octanoyl]oxymethyl]-3-[4-(2-pyrrolidin-1-ylethylcarbamoyloxy)octanoyloxy]propyl]O1-[(Z)-non-3-enyl]octanedioate

### Step 1:

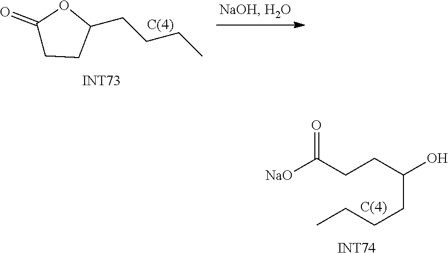

To a solution of NaOH (14.77 g, 369.21 mmol, 1.05 eq) in H₂O (250 mL) was added 5-butyltetrahydrofuran-2-one (50 g, 351.63 mmol, 50.97 mL, 1 eq) under N₂ atmosphere. The reaction mixture was stirred at 100° C. for 12 hr under N₂ atmosphere. The reaction mixture was concentrated under reduced pressure to remove solvent. Compound 4-hydroxyoctanoyloxysodium (45 g, 246.99 mmol, 70.24% yield) was obtained as a white solid and it was used in next step directly.

### Step 2:

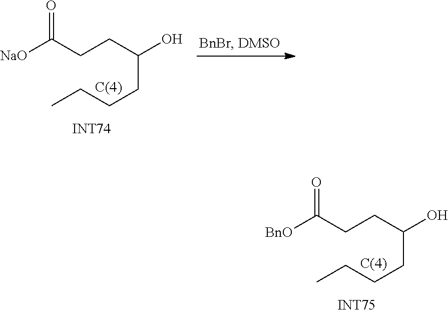

To a solution of 4-hydroxyoctanoyloxysodium (9 g, 49.40 mmol, 1 eq) in DMSO (90 mL) was added BnBr (8.45 g, 49.40 mmol, 5.87 mL, 1 eq) dropwise under N₂ atmosphere. The reaction mixture was stirred at 25° C. for 5 min under N₂ atmosphere. The reaction mixture was diluted with sat. NaCl (100 mL) and extracted with EtOAc (200 mL) (100 mL*2). The combined organic layers were dried over Na₂SO₄, filtered and the filtrate was concentrated under reduced pressure to give a residue. Compound benzyl 4-hydroxyoctanoate (2.67 g, crude) was obtained as a pale yellow oil and it was used in next step quickly.

### Step 3:

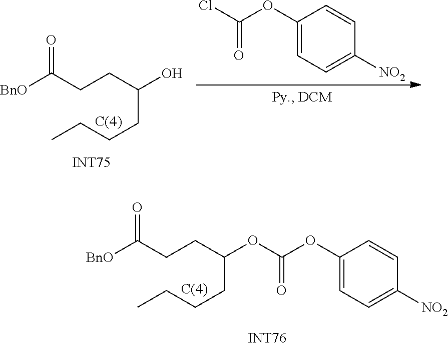

To a solution of benzyl 4-hydroxyoctanoate (8 g, 31.96 mmol, 1 eq) in DCM (201 mL) was added (4-nitrophenyl) carbonochloridate (12.88 g, 63.91 mmol, 2 eq) and Py. (5.06 g, 63.91 mmol, 5.16 mL, 2 eq) slowly at 0° C. under N₂ atmosphere. The reaction mixture was stirred at 25° C. for 1 hr under N₂ atmosphere. The reaction mixture was diluted with petroleum ether (100 mL), filtered and the filtrate was concentrated under reduced pressure to give a residue. The residue was purified by column chromatography (SiO₂, Petroleum ether/Ethyl acetate=100/1 to 10/1). Compound benzyl 4-(4-nitrophenoxy)carbonyloxyoctanoate (5 g, 12.04 mmol, 37.66% yield) was obtained as a colorless oil.

### Step 4:

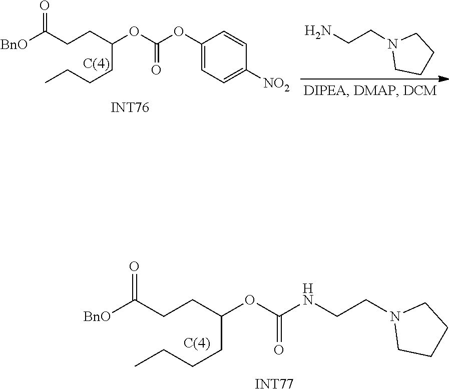

To a solution of benzyl 4-(4-nitrophenoxy)carbonyloxyoctanoate (5 g, 12.04 mmol, 1 eq) in DCM (77 mL) was added DIPEA (4.67 g, 36.11 mmol, 6.29 mL, 3 eq), 2-pyrrolidin-1-ylethanamine (2.75 g, 24.07 mmol, 2 eq) and DMAP (147.03 mg, 1.20 mmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (100 mL) and extracted with DCM 360 mL (120 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound benzyl 4-(2-pyrrolidin-1-ylethylcarbamoyloxy)octanoate (2 g, 5.12 mmol, 42.55% yield) was obtained as a colorless oil.

### Step 5:

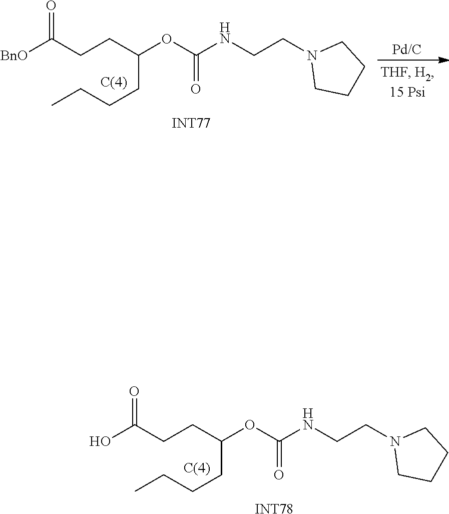

To a suspension of Pd/C (6.54 g, 6.15 mmol, 10% purity, 1.2 eq) in THF (40 mL) was added benzyl 4-(2-pyrrolidin-1-ylethylcarbamoyloxy)octanoate (2 g, 5.12 mmol, 1 eq) under N₂ atmosphere. The reaction mixture was stirred at 25° C. for 12 hr under H₂ (15 Psi). The reaction mixture was filtered and the filtrate was concentrated under reduced pressure to give a residue. The residue was purified by column chromatography (SiO₂, DCM/MeOH=50/1 to 3/1). Compound 4-(2-pyrrolidin-1-ylethylcarbamoyloxy)octanoic acid (0.5 g, 1.66 mmol, 32.50% yield) was obtained as a pale yellow oil. ¹H NMR (400 MHz, CDCl₃) δ ppm 6.56 (d, J=2.8 Hz, 1H), 4.75-4.68 (m, 1H), 3.65-3.57 (m, 1H), 3.22-3.16 (m, 1H), 3.07-2.83 (m, 6H), 2.34-2.23 (m, 2H), 2.00-1.95 (m, 5H), 1.83-1.76 (m, 1H), 1.62-1.46 (m, 2H), 1.33-1.29 (m, 4H), 0.86 (t, J=6.4 Hz, 3H).

### Step 6:

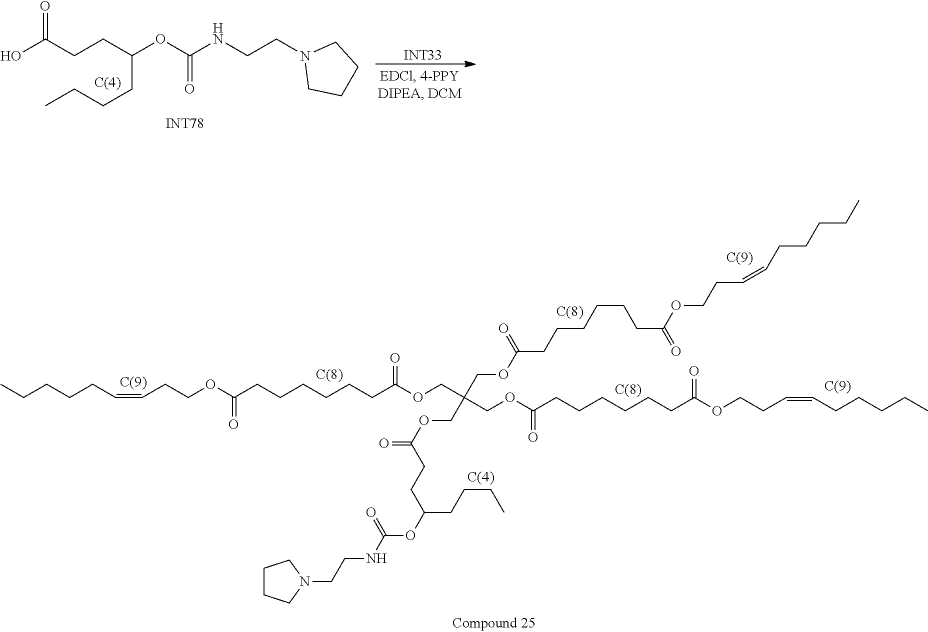

To a solution of O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (0.5 g, 511.59 mol, 1 eq) in DCM (5 mL) was added EDCI (117.69 mg, 613.91 mol, 1.2 eq), 4-(2-pyrrolidin-1-ylethylcarbamoyloxy)octanoic acid (230.52 mg, 767.38 mol, 1.5 eq), DIPEA (165.30 mg, 1.28 mmol, 222.77 L, 2.5 eq) and 4-pyrrolidin-1-ylpyridine (7.58 mg, 51.16 mol, 0.1 eq) under N₂ atmosphere. The reaction mixture was stirred at 20° C. for 12 hr under N₂ atmosphere. The reaction mixture was concentrated under reduced pressure to remove solvent. The residue was diluted with H₂O (30 mL) and extracted with DCM 60 mL (20 mL*3). The combined organic layers were dried over Na₂SO₄, filtrated, and the filtrate was concentrated under reduced pressure to give a residue. The residue was purified by column chromatography (SiO₂, Petroleum ether/Ethyl acetate=100/1 to 0/1). Compound O8-[2,2-bis[[8-[(Z)-non-3-enoxy]-8-oxo-octanoyl]oxymethyl]-3-[4-(2-pyrrolidin-1-ylethylcarbamoyloxy)octanoyloxy]propyl]O1-[(Z)-non-3-enyl]octanedioate (105 mg, 83.35 mol, 16.29% yield) was obtained as a colorless oil. ¹H NMR (400 MHz, CDCl₃) δ ppm 5.52-5.47 (m, 3H), 5.37-5.33 (m, 3H), 4.76 (s, 1H), 4.11 (s, 8H), 4.07 (t, J=7.2 Hz, 6H), 3.39 (s, 2H), 2.78 (s, 4H), 2.39-2.35 (m, 8H), 2.33-2.27 (m, 12H), 2.07-1.99 (m, 6H), 1.89-1.77 (m, 6H), 1.67-1.57 (m, 14H), 1.50-1.46 (m, 1H), 1.39-1.23 (m, 36H), 0.89 (t, J=6.8 Hz, 12H).

### Example 33—Compound 26

### O7-[2,2-bis[[7-[(Z)-non-3-enoxy]-7-oxo-heptanoyl]oxymethyl]-3-[4-(2-pyrrolidin-1-ylethylcarbamoyloxy)octanoyloxy]propyl]O1-[(Z)-non-3-enyl]heptanedioate

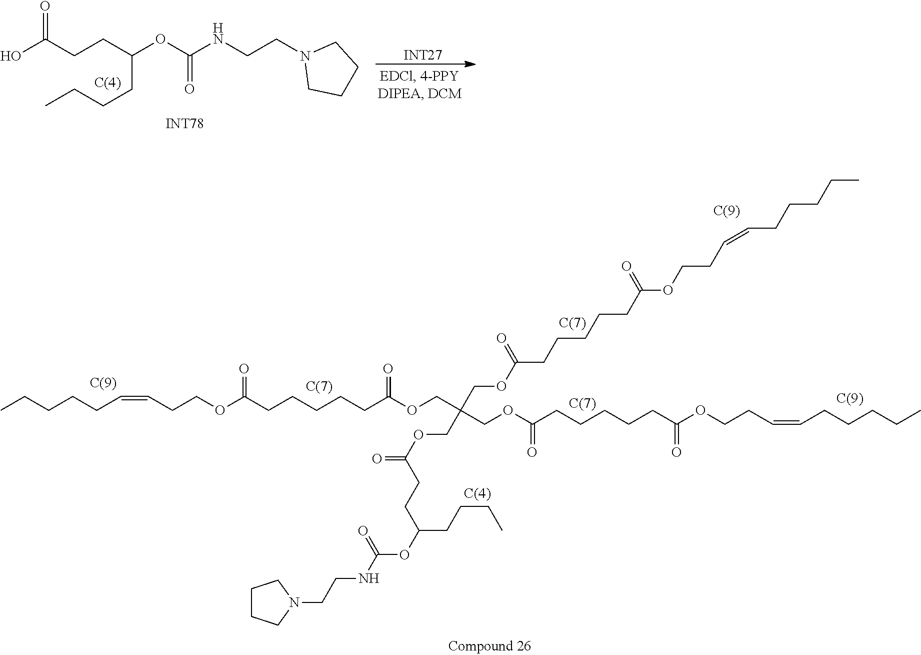

To a solution of O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl] in DCM (10 mL) was added EDCI (196.77 mg, 1.03 mmol, 1.2 eq), DIPEA (276.38 mg, 2.14 mmol, 372.47 μL, 2.5 eq), 4-(2-pyrrolidin-1-ylethylcarbamoyloxy) octanoic acid (308.34 mg, 1.03 mmol, 1.2 eq) and 4-pyrrolidin-1-ylpyridine (12.68 mg, 85.54 mol, 0.1 eq) under N₂ atmosphere. The reaction mixture was stirred at 20° C. for 12 hr under N₂ atmosphere. The reaction mixture was concentrated under reduced pressure to remove solvent. The residue was diluted with H₂O (30 mL) and extracted with DCM 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[[7-[(Z)-non-3-enoxy]-7-oxo-heptanoyl]oxymethyl]-3-[4-(2-pyrrolidin-1-ylethylcarbamoyloxy)octanoyloxy]propyl]O1-[(Z)-non-3-enyl]heptanedioate (0.12 g, 98.55 mol, 11.52% yield) was obtained as a colorless oil. ¹H NMR (400 MHz, CDCl₃) δ ppm 5.52-5.50 (m, 3H), 5.35-5.33 (m, 3H), 4.75 (s, 1H), 4.11 (s, 8H), 4.07 (t, J=7.2 Hz, 6H), 3.32 (d, J=5.2 Hz, 2H), 2.60 (d, J=25.2 Hz, 6H), 2.40-2.29 (m, 21H), 2.07-2.01 (m, 6H), 2.07-1.99 (m, 6H), 1.80 (s, 6H), 1.68-1.59 (m, 14H), 1.39-1.29 (m, 28H), 0.90 (t, J=6.8 Hz, 12H).

### Example 34—Compound 27

### O8-[2,2-bis[[8-[(Z)-non-3-enoxy]-8-oxooctanoyl]oxymethyl]-3-[4-(2-pyrrolidin-1-ylethylcarbamoyloxy)dodecanoyloxy]propyl]O1-[(Z)-non-3-enyl]octanedioate

### Step 1:

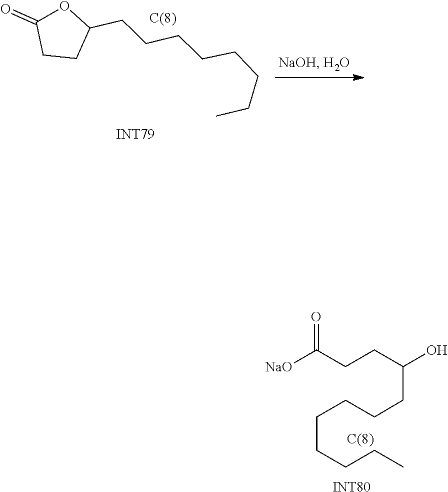

To a solution of 5-octyltetrahydrofuran-2-one (50 g, 252.14 mmol, 1 eq) in H₂O (500 mL) was added NaOH (10.59 g, 264.75 mmol, 1.05 eq) under N₂ atmosphere. The reaction mixture was stirred at 100° C. for 12 hr under N₂ atmosphere. The reaction mixture was concentrated under reduced pressure to remove solvent. Compound 4-hydroxydodecanoyloxysodium (50 g, crude) was obtained as a white solid and it was used in next step directly.

### Step 2:

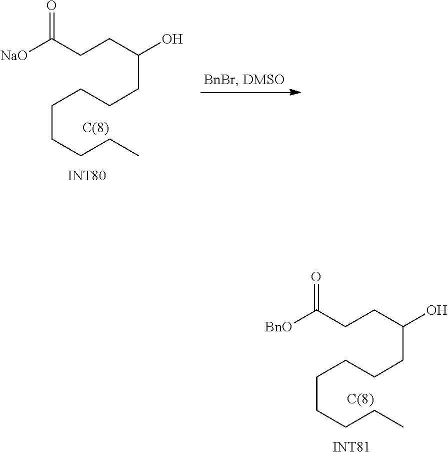

To a solution of 4-hydroxydodecanoyloxysodium (10 g, 41.96 mmol, 1 eq) in DMSO (100 mL) was added BnBr (7.18 g, 41.96 mmol, 4.98 mL, 1 eq) under N₂ atmosphere. The reaction mixture was stirred at 20° C. for 0.5 hr under N₂ atmosphere. The reaction mixture was diluted with sat. NaCl (100 mL) and extracted with EtOAc (50 mL×3). The combined organic layers were washed with brine (50 mL×2), dried over Na₂SO₄, filtered and the filtrate was concentrated under reduced pressure to give a residue. Compound benzyl 4-hydroxydecanoate (30 g, crude) was obtained as a pale yellow oil and it was used in next step quickly.

### Step 3:

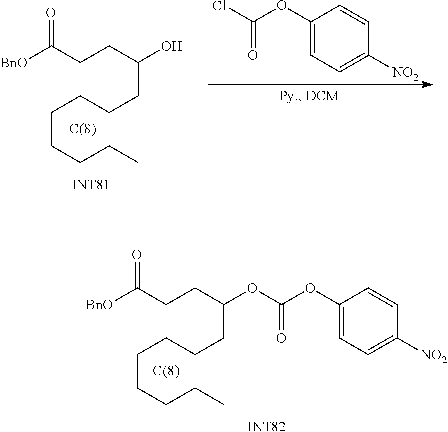

To a solution of benzyl 4-hydroxydodecanoate (10 g, 32.63 mmol, 1 eq) in DCM (100 mL) was added Py. (5.16 g, 65.27 mmol, 5.27 mL, 2 eq) and (4-nitrophenyl) carbonochloridate (7.89 g, 39.16 mmol, 1.2 eq) at 0° C. under N₂ atmosphere. The reaction mixture was stirred at 25° C. for 1 hr under N₂ atmosphere. The reaction mixture was diluted with petroleum ether (100 mL), filtered and the filtrate was concentrated under reduced pressure to give a residue. The residue was purified by column chromatography (SiO₂, Petroleum ether/Ethyl acetate=100/1 to 10/1). Compound benzyl 4-(4-nitrophenoxy)carbonyloxydodecanoate (9 g, 19.09 mmol, 81.82% yield) was obtained as a colorless oil. ¹H NMR (400 MHz, CDCl₃) δ ppm 8.28-8.24 (m, 2H), 7.41-7.33 (m, 7H), 5.14 (s, 2H), 4.92-4.85 (m, 1H), 2.52 (t, J=7.6 Hz, 2H), 2.11 (m, 1H), 2.04-1.94 (m, 1H), 1.82-1.69 (m, 1H), 1.68-1.60 (m, 1H), 1.48-1.25 (m, 12H), 0.89 (t, J=6.8 Hz, 3H).

### Step 4:

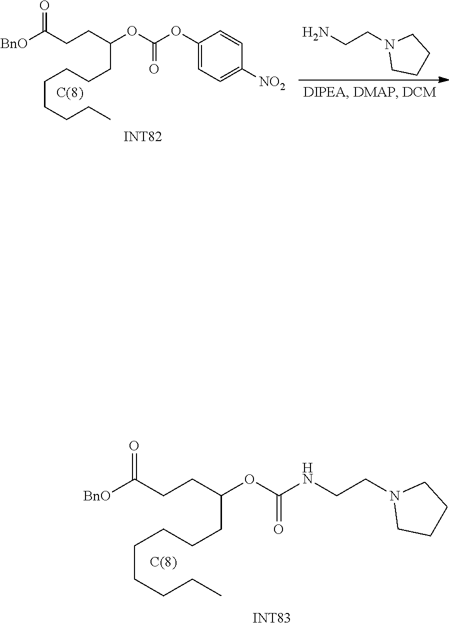

To a solution of benzyl 4-(4-nitrophenoxy)carbonyloxydodecanoate (9 g, 19.09 mmol, 1 eq) and 2-pyrrolidin-1-ylethanamine (4.36 g, 38.17 mmol, 2 eq) in DCM (90 mL) was added DIEA (7.40 g, 57.26 mmol, 9.97 mL, 3 eq) and DMAP (233.17 mg, 1.91 mmol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were concentrated under reduced pressure to give a residue. The residue was purified by column chromatography (SiO₂, Petroleum ether/Ethyl acetate=50/1 to 0/1). Compound benzyl 4-(2-pyrrolidin-1-ylethylcarbamoyloxy)dodecanoate (3 g, 6.72 mmol, 35.21% yield) was obtained as a yellow oil. ¹H NMR (400 MHz, CDCl₃) δ ppm 10.55 (br s, 1H), 7.49-7.29 (m, 5H), 5.29 (br d, J=6.2 Hz, 1H), 5.11 (s, 2H), 4.74 (br s, 1H), 3.42-3.16 (m, 2H), 2.78-2.56 (m, 6H), 2.38 (br t, J=7.6 Hz, 2H), 2.01-1.89 (m, 1H), 1.83 (br s, 4H), 1.55-1.37 (m, 2H), 1.36-1.22 (m, 12H), 0.87 (t, J=6.8 Hz, 3H).

### Step 5:

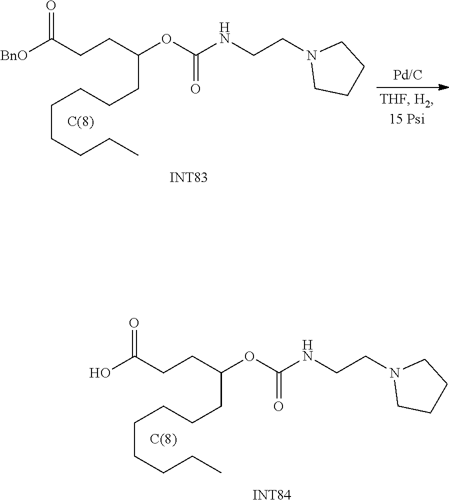

To a suspension of Pd/C (714.83 mg, 671.71 mol, 10% purity, 0.1 eq) in THF (180 mL) was added benzyl 4-(2-pyrrolidin-1-ylethylcarbamoyloxy)dodecanoate (3 g, 6.72 mmol, 1 eq) under N₂ atmosphere. The reaction mixture was stirred at 25° C. for 12 hr under H₂ (15 Psi). The reaction mixture was filtered and the filtrate was concentrated under reduced pressure to give a residue. The residue was purified by column chromatography (SiO₂, DCM/MeOH=50/1 to 3/1). Compound 4-(2-pyrrolidin-1-ylethylcarbamoyloxy)dodecanoic acid (1.8 g, 5.05 mmol, 90.00% yield) was obtained as a yellow oil. ¹H NMR (400 MHz, CDCl₃) δ ppm 8.87 (br s, 1H), 6.43 (br d, J=3.38 Hz, 1H), 4.73 (br d, J=2.50 Hz, 1H), 3.63-3.60 (m, 1H), 3.24-3.13 (m, 1H), 3.10-2.89 (m, 5H), 2.85-2.83 (m, 1H), 2.40-2.17 (m, 2H), 2.11-1.89 (m, 5H), 1.87-1.70 (m, 1H), 1.69-1.54 (m, 1H), 1.51-1.46 (m, 1H), 1.38-1.22 (m, 12H), 0.87 (t, J=6.82 Hz, 3H).

### Step 6:

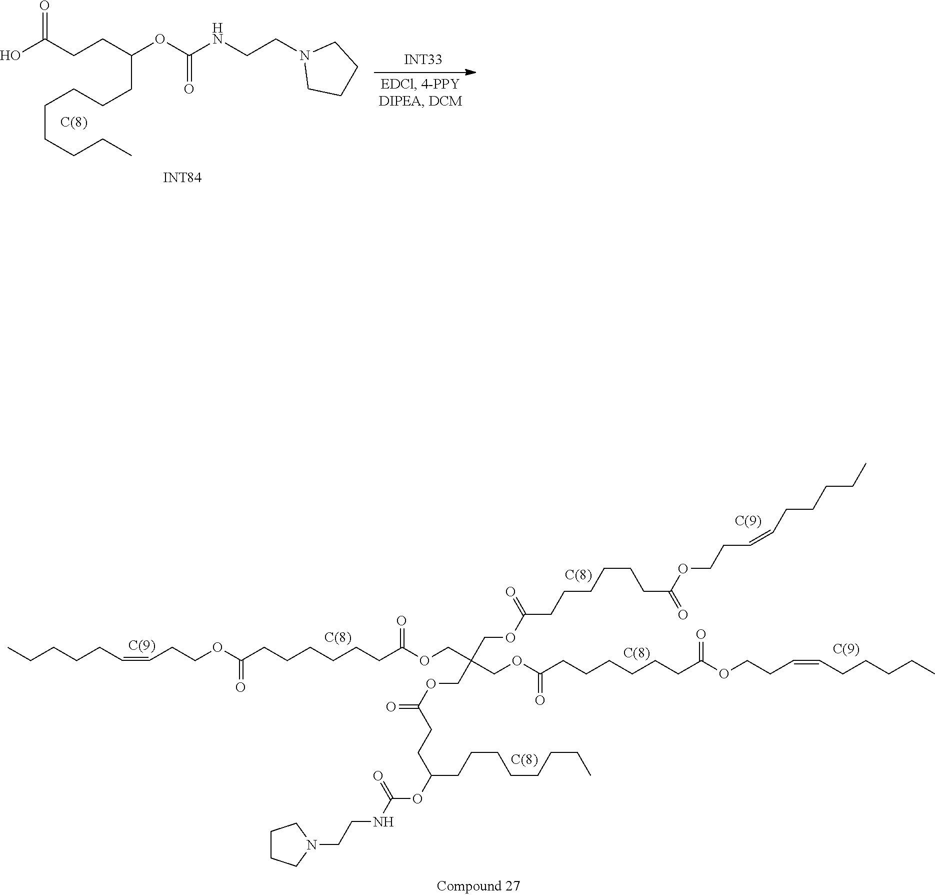

To a solution of O8-[2-(hydroxymethyl)-3-[8-[(Z)-non-3-enoxy]-8-oxo-octanoyl]oxy-2-[[8-[(Z)-non-3-enoxy]-8-oxooctanoyl]oxymethyl]propyl]O1-[(Z)-non-3-enyl]octanedioate (800 mg, 818.54 mol, 1 eq) and 4-(2-pyrrolidin-1-ylethylcarbamoyloxy)dodecanoic acid (437.71 mg, 1.23 mmol, 1.5 eq) in DCM (10 mL) was added DIEA (264.48 mg, 2.05 mmol, 356.44 L, 2.5 eq), EDCI (188.30 mg, 982.25 mol, 1.2 eq) and 4-pyrrolidin-1-ylpyridine (12.13 mg, 81.85 mol, 0.1 eq) under N₂ atmosphere. The reaction mixture was stirred at 20° C. for 12 hr under N₂ atmosphere. The reaction mixture was diluted with H₂O (30 mL) and extracted with DCM 90 mL (30 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis[[8-[(Z)-non-3-enoxy]-8-oxooctanoyl]oxymethyl]-3-[4-(2-pyrrolidin-1-ylethylcarbamoyloxy)dodecanoyloxy]propyl]O1-[(Z)-non-3-enyl]octanedioate (300 mg, 183.06 mol, 22.30% yield, 80.29% purity) was obtained as a colorless oil. ¹H NMR (400 MHz, CDCl₃) δ ppm 5.52-5.49 (m, 3H), 5.37-5.33 (m, 3H), 4.75 (br s, 1H), 4.18-4.02 (m, 14H), 3.35 (br d, J=4.8 Hz, 2H), 2.81-2.48 (m, 6H), 2.37 (m, 8H), 2.04 (m, 6H), 2.30 (m, 12H), 1.94-1.78 (m, 6H), 1.71-1.58 (m, 12H), 1.43-1.10 (m, 46H), 0.91-0.84 (m, 12H).

### Example 35—Compound 28

### O7-[2,2-bis[[7-[(Z)-non-3-enoxy]-7-oxo-heptanoyl]oxymethyl]-3-[4-(2-pyrrolidin-1-ylethylcarbamoyloxy)dodecanoyloxy]propyl]O1-[(Z)-non-3-enyl]heptanedioate

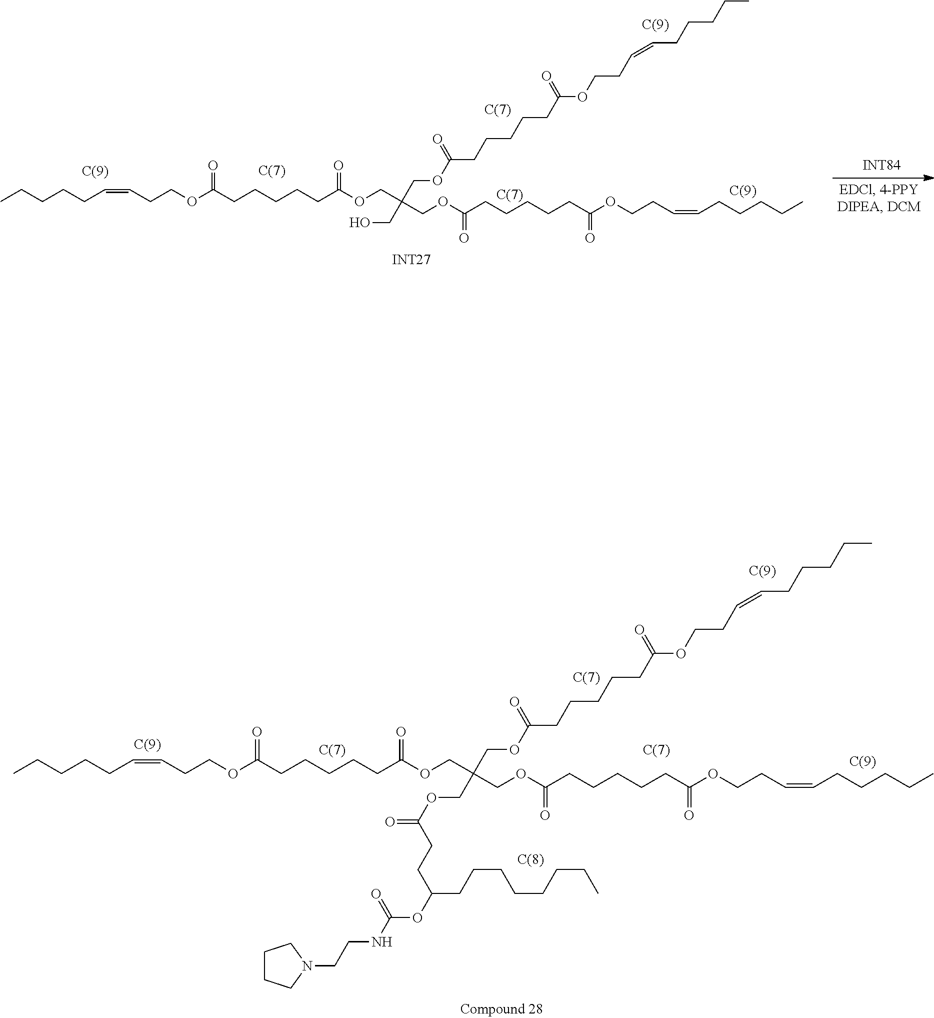

To a solution of O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (0.8 g, 855.37 mol, 1 eq) in DCM (110 mL) was added EDCI (196.77 mg, 1.03 mmol, 1.2 eq), 4-(2-pyrrolidin-1-ylethylcarbamoyloxy)dodecanoic acid (274.44 mg, 769.83 mol, 0.9 eq), DMAP (261.24 mg, 2.14 mmol, 2.5 eq) and 4-pyrrolidin-1-ylpyridine (12.68 mg, 85.54 mol, 0.1 eq) under N₂ atmosphere. The reaction mixture was stirred at 20° C. for 12 hr under N₂ atmosphere. The reaction mixture was concentrated under reduced pressure to remove solvent. The residue was diluted with H₂O (200 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[[7-[(Z)-non-3-enoxy]-7-oxo-heptanoyl]oxymethyl]-3-[4-(2-pyrrolidin-1-ylethylcarbamoyloxy)dodecanoyloxy]propyl]O1-[(Z)-non-3-enyl]heptanedioate (0.12 g, 94.21 mol, 11.01% yield) was obtained as a colorless oil. ¹H NMR (400 MHz, CDCl₃) δ ppm 5.49-5.52 (m, 3H), 5.35-5.32 (m, 3H), 4.67 (s, 1H), 4.03 (s, 8H), 3.99 (t, J=7.2 Hz, 6H), 3.70-3.26 (m, 2H), 2.60-2.46 (m, 6H), 2.33-2.21 (m, 20H), 1.99-1.91 (m, 6H), 1.75 (s, 6H), 1.58-1.53 (m, 14H), 1.31-1.19 (m, 36H), 0.84-0.79 (m, 12H).

### Example 36—Compound 29

### O8-[2-[4-[2-[ethyl(methyl)amino]ethylcarbamoyloxy]decanoyloxymethyl]-3-[8-[(Z)-non-3-enoxy]-8-oxo-octanoyl]oxy-2-[[8-[(Z)-non-3-enoxy]-8-oxooctanoyl]oxymethyl]propyl]O1-[(Z)-non-3-enyl]octanedioate

### Step 1:

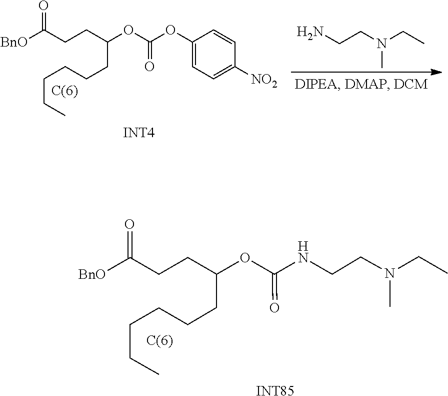

To a mixture of benzyl 4-(4-nitrophenoxy)carbonyloxydecanoate (9 g, 20.29 mmol, 1 eq), DIPEA (7.87 g, 60.88 mmol, 10.60 mL, 3 eq) and DMAP (247.92 mg, 2.03 mmol, 0.1 eq) in DCM (90 mL) was added N′-ethyl-N′-methyl-ethane-1,2-diamine (5.18 g, 50.73 mmol, 2.5 eq) under N₂ atmosphere. The reaction mixture was stirred at 25° C. for 12 hr under N₂ atmosphere. The reaction mixture was diluted with H₂O (40 mL) and extracted with ethyl acetate 120 mL (40 mL*3). The combined organic layers were concentrated under reduced pressure to give a residue. The residue was purified by column chromatography (SiO₂, Petroleum ether/Ethyl acetate=100/1 to 0/1). Compound benzyl 4-[2-[ethyl(methyl)amino]ethylcarbamoyloxy]decanoate (5 g, 12.30 mmol, 60.60% yield) was obtained as a colorless oil. ¹H NMR (400 MHz, MeOD) δ ppm 7.31-7.28 (m, 5H), 4.86 (s, 2H), 4.77-4.71 (m, 1H), 3.23 (t, J=6.8 Hz, 2H), 2.60-2.47 (m, 4H), 2.42 (t, J=7.4 Hz, 2H), 2.29 (s, 3H), 1.92 (m, 1H), 1.80 (m, 1H), 1.64-1.48 (m, 2H), 1.30 (br d, J=5.2 Hz, 8H), 1.08 (t, J=7.2 Hz, 3H), 0.95-0.82 (m, 3H).

### Step 2:

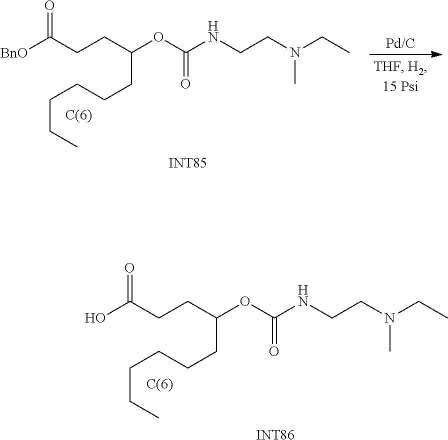 [SMILES: CCCCCCC(CCC(=O)OCc1ccccc1)OC(=O)NCCN(C)CC ; "INT85" ; CCCCCCC(CCC(=O)O)OC(=O)NCCN(C)CC ; "INT86"]

To a suspension of Pd/C (300 mg, 10% purity) in THF (100 mL) was added benzyl 4-[2-[ethyl(methyl)amino]ethylcarbamoyloxy]decanoate (5 g, 12.30 mmol, 1 eq) under N₂ atmosphere. The reaction mixture was stirred at 25° C. for 12 hr under H₂ (15 Psi). The reaction mixture was filtered and the filtrate was concentrated under reduced pressure to give a residue. The residue was purified by column chromatography (SiO₂, Petroleum ether/Ethyl acetate=100/1 to DCM:MeOH=0:1). Compound 4-[2-[ethyl(methyl)amino]ethylcarbamoyloxy]decanoic acid (2.2 g, 6.95 mmol, 56.53% yield) was obtained as a pale yellow oil. ¹H NMR (400 MHz, MeOD) δ ppm 4.84-4.70 (m, 1H), 3.31-3.26 (m, 1H), 3.23-3.01 (m, 5H), 2.75 (s, 3H), 2.27-2.20 (m, 1H), 2.14-1.98 (m, 2H), 1.80-1.72 (m, 1H), 1.61-1.57 (m, 1H), 1.48-1.44 (m, 1H), 1.30-1.27 (m, 10H), 0.88-0.84 (m, 3H).

### Step 3:

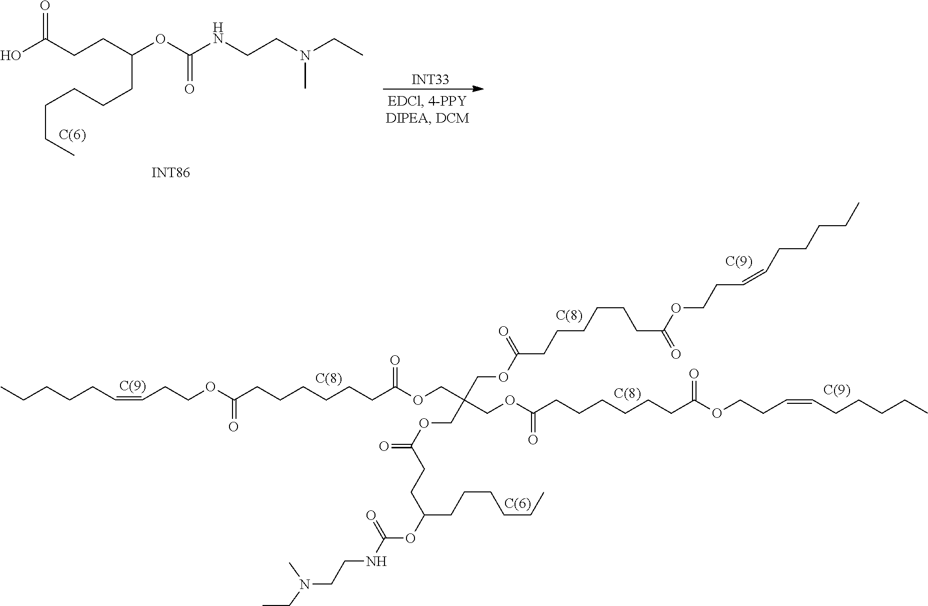

To a mixture of O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (1.1 g, 1.13 mmol, 1 eq), 4-[2-[ethyl(methyl)amino]ethylcarbamoyloxy]decanoic acid (427.37 mg, 1.35 mmol, 1.2 eq), DIPEA (363.66 mg, 2.81 mmol, 490.10 L, 2.5 eq) and EDCI (258.91 mg, 1.35 mmol, 1.2 eq) in DCM (20 mL) was added 4-pyrrolidin-1-ylpyridine (16.68 mg, 112.55 mol, 0.1 eq) under N₂ atmosphere. The reaction mixture was stirred at 25° C. for 12 hr under N₂ atmosphere. The reaction mixture was diluted with H₂O (20 mL) and extracted with EtOAc 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). O8-[2-[4-[2-[ethyl(methyl)amino]ethylcarbamoyloxy]decanoyloxymethyl]-3-[8-[(Z)-non-3-enoxy]-8-oxo-octanoyl]oxy-2-[[8-[(Z)-non-3-enoxy]-8-oxooctanoyl]oxymethyl]propyl]O1-[(Z)-non-3-enyl]octanedioate (100 mg, 78.38 mol, 6.96% yield, 100% purity) was obtained as a colorless oil. ¹H NMR (400 MHz, CDCl₃) δ ppm 5.52-5.49 (m, 3H), 5.35-5.33 (m, 3H), 4.75 (s, 1H), 4.11 (s, 8H), 4.06 (t, J=3.4 Hz, 6H), 3.29 (s, 2H), 2.53-2.37 (m, 4H), 2.40-2.33 (m, 8H), 2.31-2.78 (m, 14H), 2.07-2.01 (m, 6H), 1.64-1.57 (m, 14H), 1.50-1.40 (m, 2H), 1.36-1.27 (m, 40H), 1.10-1.05 (m, 3H), 0.91-0.86 (m, 12H).

### Example 37—Compound 30

### O7-[2-[4-[2-[ethyl(methyl)amino]ethylcarbamoyloxy]decanoyloxymethyl]-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate

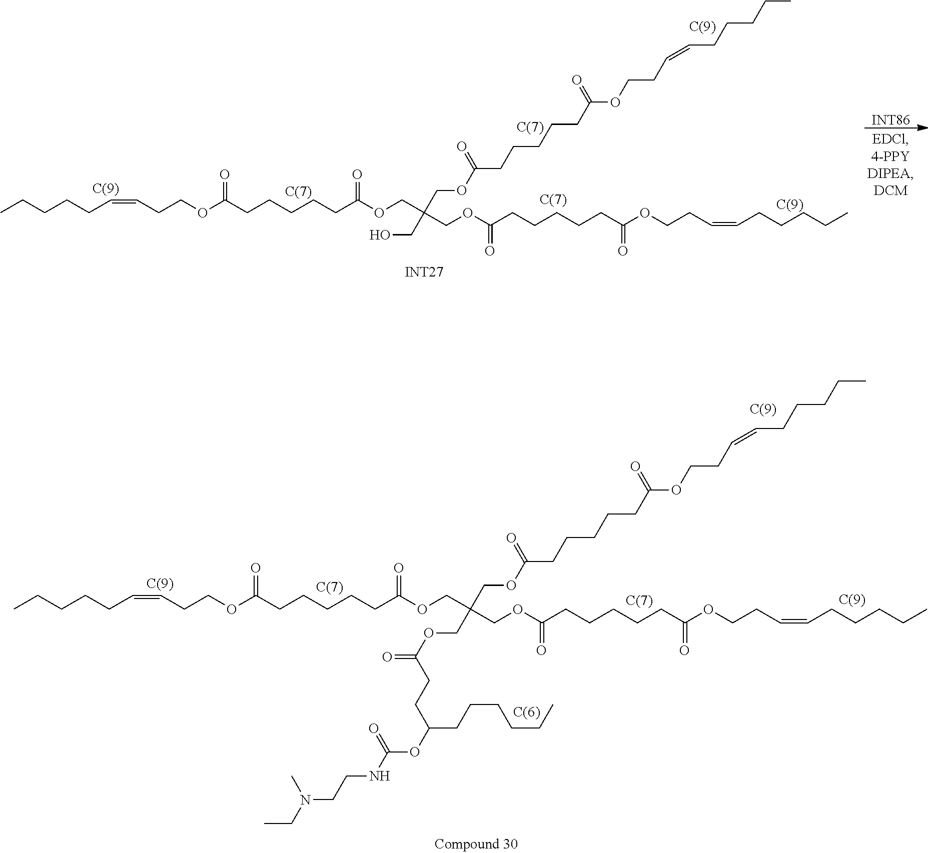

To a mixture of O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (0.8 g, 855.37 mol, 1 eq), 4-[2-[ ethyl(methyl)amino]ethylcarbamoyloxy]decanoic acid (324.80 mg, 1.03 mmol, 1.2 eq), DIPEA (276.38 mg, 2.14 mmol, 372.47 μL, 2.5 eq) and EDCI (196.77 mg, 1.03 mmol, 1.2 eq) in DCM (10 mL) was added 4-pyrrolidin-1-ylpyridine (12.68 mg, 85.54 mol, 0.1 eq) under N₂ atmosphere. The reaction mixture was stirred at 25° C. for 12 hr under N₂ atmosphere. The reaction mixture was diluted with H₂O (20 mL) and extracted with EtOAc 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). O7-[2-[4-[2-[ethyl(methyl)amino]ethylcarbamoyloxy]decanoyloxymethyl]-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (0.1 g, 81.06 mol, 9.48% yield, 100% purity) was obtained as a colorless oil. ¹H NMR (400 MHz, CDCl₃) δ ppm 5.52-5.50 (m, 3H), 5.36-5.33 (m, 3H), 4.75 (s, 1H), 4.11 (s, 8H), 4.06 (t, J=3.4 Hz, 6H), 3.37 (s, 2H), 2.65-2.37 (m, 3H), 2.40-2.34 (m, 22H), 1.68-1.60 (m, 22H), 1.38-1.24 (m, 32H), 0.89 (m, 12H).

### Example 38—Compound 31

### O8-[2,2-bis[[8-[(Z)-non-3-enoxy]-8-oxo-octanoyl]oxymethyl]-3-[4-(3-pyrrolidin-1-ylpropoxycarbonyloxy)decanoyloxy]propyl]O1-[(Z)-non-3-enyl]octanedioate

### Step 1:

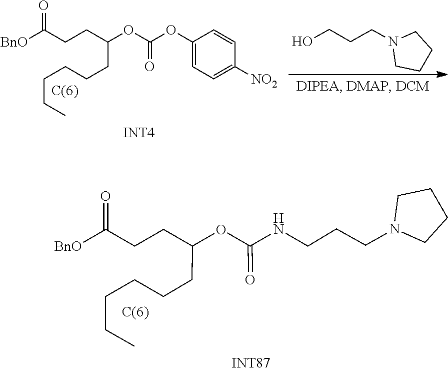

To a solution of benzyl 4-(4-nitrophenoxy)carbonyloxydecanoate (2 g, 4.51 mmol, 1 eq) in ACN (28 mL) was added Py. (713.43 mg, 9.02 mmol, 727.99 L, 2 eq), 3-pyrrolidin-1-ylpropan-1-ol (873.98 mg, 6.76 mmol, 1.5 eq) and DMAP (55.09 mg, 450.97 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (100 mL) and extracted with EtOAc 400 mL (100 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound benzyl 4-(3-pyrrolidin-1-ylpropoxycarbonyloxy)decanoate (1 g, 2.31 mmol, 51.14% yield) was obtained as a colorless oil. ¹H NMR (400 MHz, CDCl3) δ ppm 7.37-7.27 (m, 5H), 5.12 (s, 2H), 4.75 -4.72 (m, 1H), 4.20-4.16 (m, 2H), 2.59-2.55 (m, 2H), 2.64-2.49 (m, 6H), 1.99 (m, 1H), 1.95-1.56 (m, 3H), 1.89-1.81 (m, 4H), 1.70-1.48 (m, 2H), 1.38-1.25 (m, 8H), 0.88 (t, J=6.6 Hz, 3H).

### Step 2:

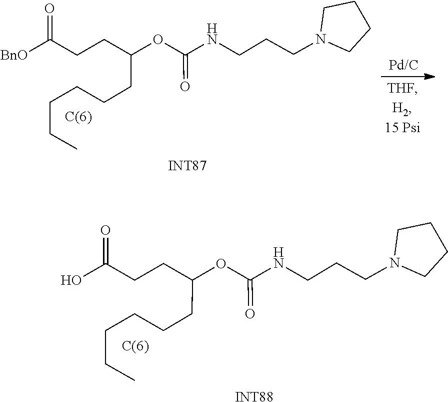 [SMILES: CCCCCCC(CCC(=O)OCc1ccccc1)OC(=O)NCCCN1CCCC1 ; "INT87" ; CCCCCCC(CCC(=O)O)OC(=O)NCCCN1CCCC1 ; "INT88"]

To a suspension of Pd/C (333.33 mg, 313.22 mol, 10% purity, 1.36e-1 eq) in THF (20 mL) was added benzyl 4-(3-pyrrolidin-1-ylpropoxycarbonyloxy) decanoate (1 g, 2.31 mmol, 1 eq) under N₂ atmosphere. The reaction mixture was stirred under H₂ (15 Psi.) at 25° C. for 12 hr. The reaction mixture was filtered and the filtrate was concentrated under reduced pressure to give a residue. The residue was purified by column chromatography (SiO2, DCM/MeOH=100/1 to 0/1). Compound 4-(3-pyrrolidin-1-ylpropoxycarbonyloxy)decanoic acid (0.4 g, 1.16 mmol, 50.50% yield) was obtained as a pale yellow oil. ¹H NMR (400 MHz, CDCl₃) δ ppm 8.24-8.17 (m, 1H), 4.84-4.82 (m, 1H), 4.34-4.32 (m, 1H), 4.02 (m, 1H), 3.32-3.05 (m, 3H), 2.98-2.87 (m, 2H), 2.26-2.73 (m, 1H), 2.39-2.20 (m, 2H), 2.15-2.03 (m, 1H), 2.01-1.83 (m, 7H), 1.78-1.56 (m, 1H), 1.52 (m, 1H), 1.38-1.07 (m, 8H), 0.95-0.76 (m, 3H).

### Step 3:

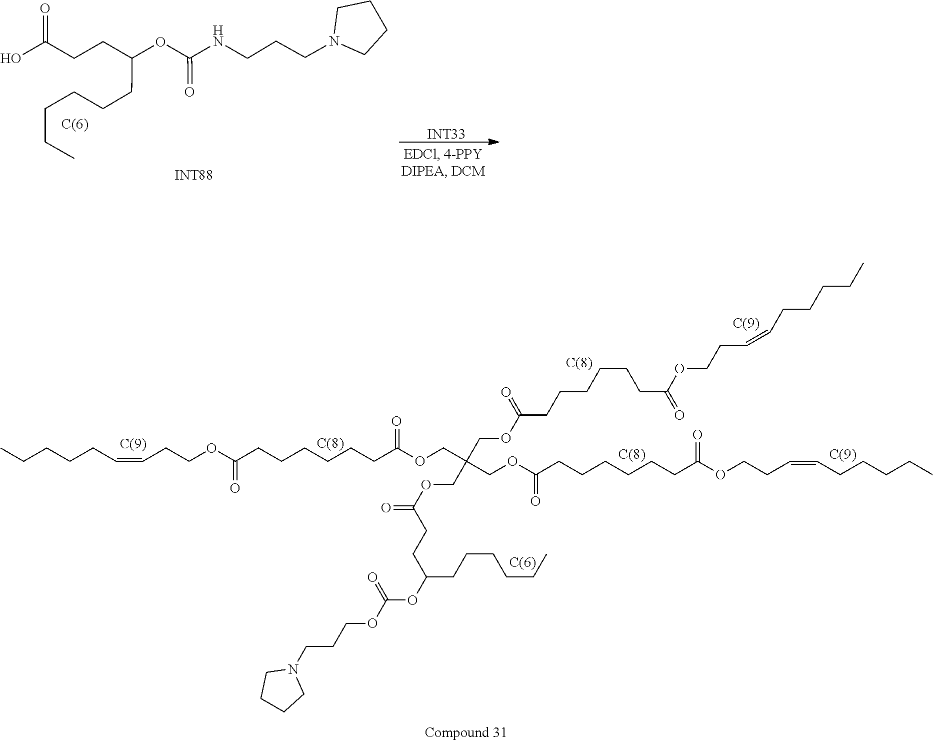 [SMILES: CCCCCCC(CCC(=O)O)OC(=O)NCCCN1CCCC1 ; "INT88" ; "INT33" ; CCCCC/C=C\CCOC(=O)CCCCCCC(=O)OCC(COC(=O)CCCCCCC(=O)OCC/C=C\CCCCC)(COC(=O)CCCCCCC(=O)OCC/C=C\CCCCC)COC(=O)CCC(CCCCCC)OC(=O)OCCCN1CCCC1]

To a solution of O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (1 g, 1.02 mmol, 1 eq) in DCM (1.13 mL) was added EDCI (235.37 mg, 1.23 mmol, 1.2 eq), 4-(3-pyrrolidin-1-ylpropoxycarbonyloxy)decanoic acid (316.27 mg, 920.86 mol, 0.9 eq), 4-pyrrolidin-1-ylpyridine (15.16 mg, 102.32 mol, 0.1 eq) and DIPEA (330.60 mg, 2.56 mmol, 445.55 L, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O8-[2,2-bis[[8-[(Z)-non-3-enoxy]-8-oxo-octanoyl]oxymethyl]-3-[4-(3-pyrrolidin-1-ylpropoxycarbonyloxy)decanoyloxy]propyl]O1-[(Z)-non-3-enyl]octanedioate (0.1 g, 76.76 mol, 7.50% yield) was obtained as a colorless oil. ¹H NMR (400 MHz, CDCl₃) δ ppm 5.54-5.48 (m, 3H), 5.37-5.31 (m, 3H), 4.72-4.70 (m, 1H), 4.21-4.20 (m, 2H), 4.11 (d, J=4.0 Hz, 8H), 4.06 (t, J=7.2 Hz, 6H), 2.63 (s, 4H), 2.40-2.35 (m, 8H), 2.33-2.28 (m, 12H), 2.05-2.01 (m, 10H), 1.88-1.84 (m, 4H), 1.64-1.59 (m, 16H), 1.34-1.28 (m, 38H), 0.91-0.87 (m, 12H).

### Example 39—Compound 32

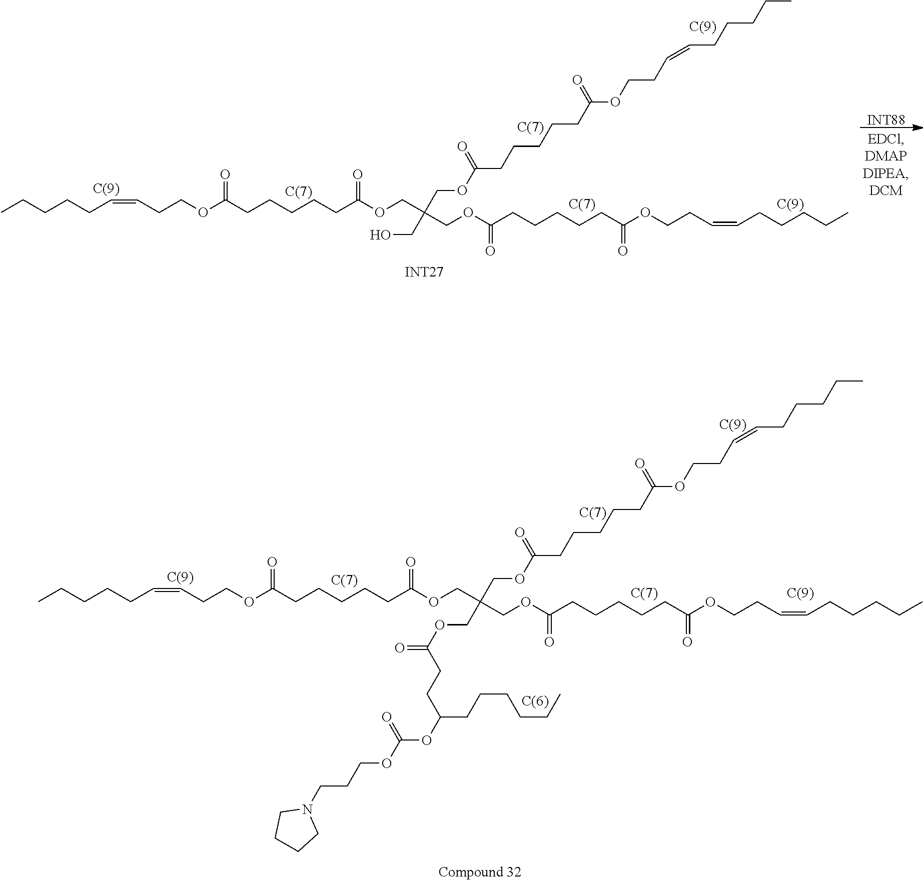

To a solution of O7-[2-(hydroxymethyl)-3-[7-[(Z)-non-3-enoxy]-7-oxo-heptanoyl]oxy-2-[[7-[(Z)-non-3-enoxy]-7-oxo-heptanoyl]oxymethyl]propyl]O1-[(Z)-non-3-enyl]heptanedioate (1.5 g, 1.60 mmol, 1 eq) in DCM (20 mL) was added EDCI (368.94 mg, 1.92 mmol, 1.2 eq), 4-(3-pyrrolidin-1-ylpropoxycarbonyloxy)decanoic acid (495.76 mg, 1.44 mmol, 0.9 eq), DMAP (19.59 mg, 160.38 mol, 0.1 eq) and DIPEA (518.20 mg, 4.01 mmol, 698.39 L, 2.5 eq) under N₂ atmosphere. The reaction mixture was stirred at 25° C. for 12 hr under N₂ atmosphere. The reaction mixture was diluted with H₂O (100 mL) and extracted with DCM 200 (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2,2-bis[[7-[(Z)-non-3-enoxy]-7-oxo-heptanoyl]oxymethyl]-3-[4-(3-pyrrolidin-1-ylpropoxycarbonyloxy) decanoyloxy]propyl]O1-[(Z)-non-3-enyl]heptanedioate (100 mg, 78.88 mol, 4.92% yield, 99.45% purity) was obtained as a colorless oil. ¹H NMR (400 MHz, CDCl₃) δ ppm 5.52-5.50 (m, 3H), 5.35-5.33 (m, 3H), 4.74-4.70 (m, 1H), 4.22 (t, J=6 Hz, 2H), 4.11 (s, 8H), 4.06 (t, J=7.2 Hz, 6H), 3.04-2.88 (m, 4H), 2.38 (t, J=6.4 Hz, 7H), 2.35-2.27 (m, 12H), 2.20-2.18 (m, 2H), 2.07-2.01 (m, 10H), 1.66-1.60 (m, 20H), 1.37-1.28 (m, 30H), 0.91-0.87 (m, 12H).

### Example 40—Compound 33

### O8-[2,2-bis[(8-octoxy-8-oxo-octanoyl)oxymethyl]-3-[4-(2-pyrrolidin-1-ylethylcarbamoyloxy)decanoyloxy]propyl]O1-octyl octanedioate

### Step 1:

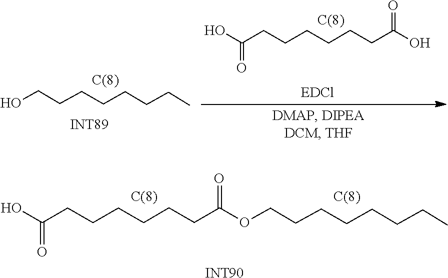

To a mixture of octan-1-ol (20 g, 153.58 mmol, 24.27 mL, 1 eq), DIPEA (59.55 g, 460.73 mmol, 80.25 mL, 3 eq), DMAP (3.75 g, 30.72 mmol, 0.2 eq) and EDCI (38.27 g, 199.65 mmol, 1.3 eq) in DCM (800 mL) and THF (800 mL) was added octanedioic acid (133.76 g, 767.89 mmol, 5 eq) under N₂ atmosphere and the reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 8-octoxy-8-oxo-octanoic acid (25 g, 87.29 mmol, 56.84% yield) was obtained as a colorless oil. ¹H NMR (400 MHz, CDCl₃) δ ppm 11.23 (br s, 1H), 4.06 (t, J=6.8 Hz, 2H), 2.42-2.22 (m, 4H), 1.73-1.55 (m, 6H), 1.46-1.26 (m, 14H), 0.89 (t, J=6.8 Hz, 3H).

### Step 2:

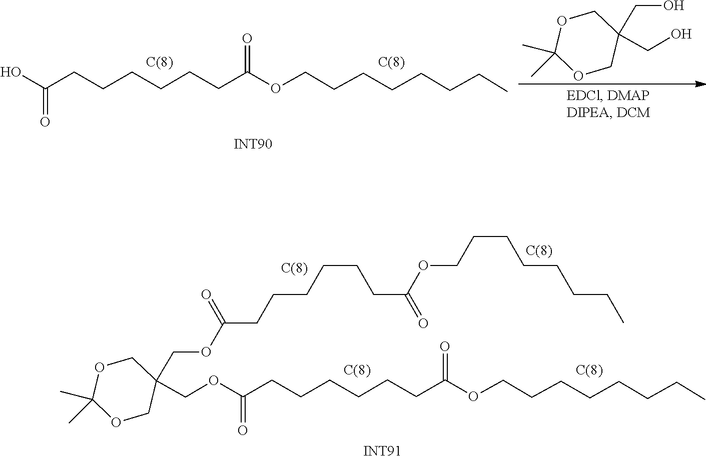

To a mixture of 8-octoxy-8-oxo-octanoic acid (14.45 g, 50.44 mmol, 1 eq), DIPEA (16.30 g, 126.11 mmol, 21.97 mL, 2.5 eq), EDCI (11.60 g, 60.53 mmol, 1.2 eq) and DMAP (616.27 mg, 5.04 mmol, 0.1 eq) in DCM (200 mL) was added [5-(hydroxymethyl)-2,2 -dimethyl-1,3-dioxan-5-yl]methanol (4 g, 22.70 mmol, 0.45 eq) under N₂ atmosphere and the reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[[2,2-dimethyl-5-[(8-octoxy-8-oxo-octanoyl)oxymethyl]-1,3-dioxan-5-yl]methyl]O1-octyl octanedioate (6.5 g, 9.12 mmol, 18.07% yield) was obtained as a colorless oil. ¹H NMR (400 MHz, CDCl₃) δ ppm 4.10 (s, 4H), 4.06 (t, J=6.8 Hz, 4H), 3.74 (s, 4H), 2.30 (m, 8H), 1.69-1.59 (m, 12H), 1.42 (s, 6H), 1.36-1.24 (m, 28H), 0.94-0.83 (m, 6H).

### Step 3:

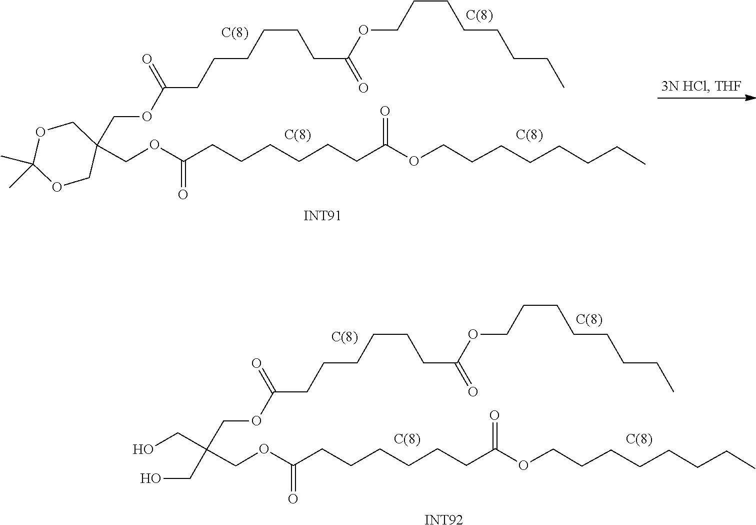

To a solution of O8-[[2,2-dimethyl-5-[(8-octoxy-8-oxo-octanoyl)oxymethyl]-1,3-dioxan-5-yl]methyl]O1-octyl octanedioate (6.5 g, 9.12 mmol, 1 eq) in THF (65 mL) was added HCl (3 M, 3.75 mL, 1.1 eq) at 0° C. under N₂ atmosphere. The reaction mixture was stirred at 25° C. for 12 hr under N₂ atmosphere. The reaction mixture was diluted with aq. NaHCO₃ (200 mL) and extracted with EtOAc 250 mL (50 mL*5). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis(hydroxymethyl)-3-(8-octoxy-8-oxo-octanoyl)oxy-propyl]O1-octyl octanedioate (2.5 g, 3.72 mmol, 40.75% yield) was obtained as a colorless oil. ¹H NMR (400 MHz, CDCl₃) δ ppm 4.15 (s, 4H), 3.98 (t, J=6.8 Hz, 4H), 3.57 (s, 4H), 2.86 (br s, 2H), 2.34-2.16 (m, 8H), 1.59-1.46 (m, 12H), 1.31-1.17 (m, 28H), 0.90-0.72 (m, 6H).

### Step 4:

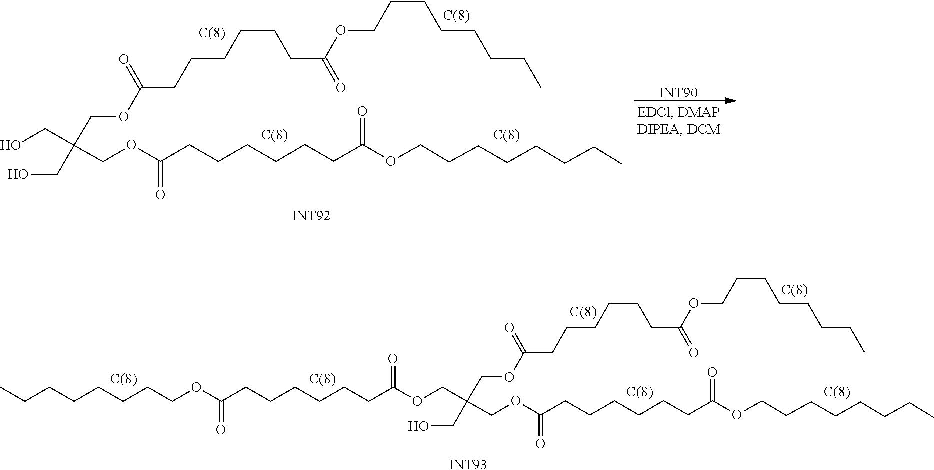

To a mixture of O8-[2,2-bis(hydroxymethyl)-3-(8-octoxy-8-oxo-octanoyl)oxy-propyl]O1-octyl octanedioate (1.3 g, 1.93 mmol, 1 eq), EDCI (444.41 mg, 2.32 mmol, 1.2 eq), DMAP (23.60 mg, 193.19 mol, 0.1 eq) and DIPEA (624.20 mg, 4.83 mmol, 841.24 L, 2.5 eq) in DCM (150 mL) was added 8-octoxy-8-oxo-octanoic acid (331.98 mg, 1.16 mmol, 0.6 eq) under N₂ atmosphere and the reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2-(hydroxymethyl)-3-(8-octoxy-8-oxo-octanoyl)oxy-2-[(8-octoxy-8-oxo-octanoyl)oxymethyl]propyl]O1-octyl octanedioate (500 mg, 531.17 mol, 27.50% yield) was obtained as a colorless oil. ¹H NMR (400 MHz, CDCl₃) δ ppm 4.11 (s, 6H), 4.06 (t, J=6.8 Hz, 6H), 3.50 (d, J=6.8 Hz, 2H), 2.60 (t, J=6.8 Hz, 1H), 2.38-2.27 (m, 12H), 1.62 (m, 18H), 1.36-1.23 (m, 42H), 0.93-0.82 (m, 9H).

### Step 5:

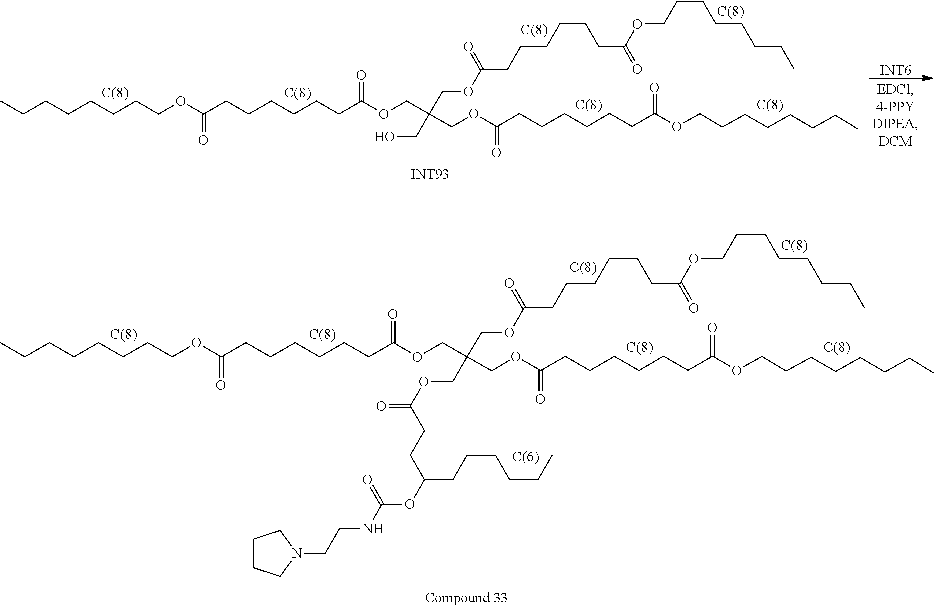

To a mixture of O8-[2-(hydroxymethyl)-3-(8-octoxy-8-oxo-octanoyl)oxy-2-[(8-octoxy-8-oxo-octanoyl)oxymethyl]propyl]O1-octyl octanedioate (1.1 g, 1.17 mmol, 1 eq), DIPEA (377.58 mg, 2.92 mmol, 508.86 L, 2.5 eq), EDCI (268.82 mg, 1.40 mmol, 1.2 eq) and 4-pyrrolidin-1-ylpyridine (17.32 mg, 116.86 mol, 0.1 eq) in DCM (15 mL) was added 4-(2-pyrrolidin-1-ylethylcarbamoyloxy)decanoic acid (498.96 mg, 1.52 mmol, 1.3 eq) under N₂ atmosphere and the reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis[(8-octoxy-8-oxo-octanoyl)oxymethyl]-3-[4-(2-pyrrolidin-1-ylethylcarbamoyloxy)decanoyloxy]propyl]O1-octyl octanedioate (150 mg, 119.83 mol, 10.25% yield, 100% purity) was obtained as a colorless oil. ¹H NMR (400 MHz, CDCl₃) δ ppm 5.18 (s, 1H), 4.75 (s, 1H), 4.14-4.11 (m, 8H), 4.05 (t, J=3.4 Hz, 2H), 3.29 (d, J=2.6 Hz, 2H), 2.60-2.52 (m, 6H), 2.33-2.29 (m, 14H), 1.90 (d, J=3.0 Hz, 1H), 1.18 (s, 4H), 1.62-1.61 (m, 20H), 1.35-1.25 (m, 52H), 0.90-0.86 (m, 12H).

### Example 41—Compound 34

### O7-[2,2-bis[(7-octoxy-7-oxo-heptanoyl)oxymethyl]-3-[4-(2-pyrrolidin-1-ylethylcarbamoyloxy) decanoyloxy]propyl]O1-octyl heptanedioate

### Step 1:

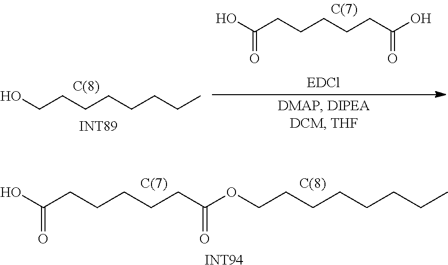

A solution of heptanedioic acid (122.99 g, 767.89 mmol, 5 eq) in DCM (615 mL) and THF (615 mL) was added EDCI (38.27 g, 199.65 mmol, 1.3 eq), DMAP (1.88 g, 15.36 mmol, 0.1 eq), DIPEA (49.62 g, 383.94 mmol, 66.88 mL, 2.5 eq) and octan-1-ol (20 g, 153.58 mmol, 24.27 mL, 1 eq) was stirred at 25° C. for 12 hr under N₂ atmosphere. The reaction mixture was diluted with H₂O (300 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound 7-octoxy-7-oxo-heptanoic acid (30 g, 110.14 mmol, 71.72% yield) was obtained as a colorless oil. ¹H NMR (400 MHz, CDCl₃) δ ppm 11.02 (br s, 1H), 4.06 (t, J=6.8 Hz, 2H), 2.43-2.24 (m, 4H), 1.74-1.56 (m, 6H), 1.45-1.16 (m, 12H), 0.94-0.80 (m, 3H).

### Step 2:

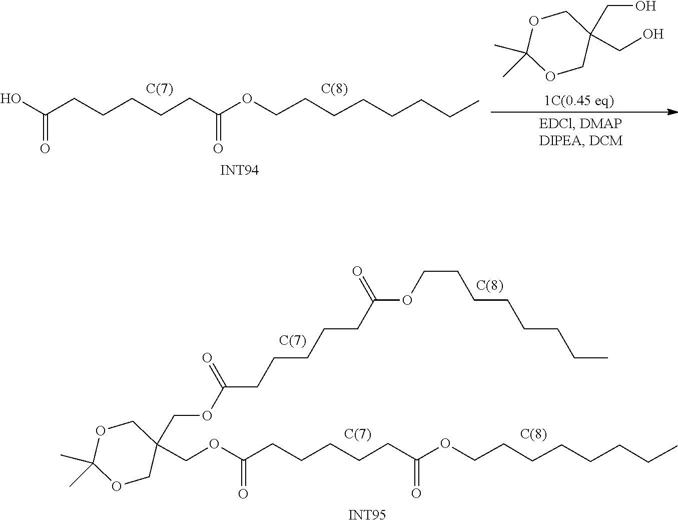

To a solution of 7-octoxy-7-oxo-heptanoic acid (13.74 g, 50.44 mmol, 1 eq) in DCM (130 mL) was added EDCI (11.60 g, 60.53 mmol, 1.2 eq), [5-(hydroxymethyl)-2,2-dimethyl-1,3-dioxan-5-yl]methanol (4 g, 22.70 mmol, 0.45 eq), DMAP (616.27 mg, 5.04 mmol, 0.1 eq) and DIPEA (16.30 g, 126.11 mmol, 21.97 mL, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[[2,2-dimethyl-5-[(7-octoxy-7-oxo-heptanoyl)oxymethyl]-1,3-dioxan-5-yl]methyl]O1-octylheptanedioate (7 g, 10.22 mmol, 20.26% yield) was obtained as a colorless oil. ¹H NMR (400 MHz, CDCl₃) δ ppm 4.11 (s, 4H), 4.06 (t, J=6.8 Hz, 4H), 3.74 (s, 4H), 2.32 (m, 8H), 1.70-1.59 (m, 12H), 1.42 (s, 6H), 1.40-1.25 (m, 24H), 0.95-0.81 (m, 6H).

### Step 3:

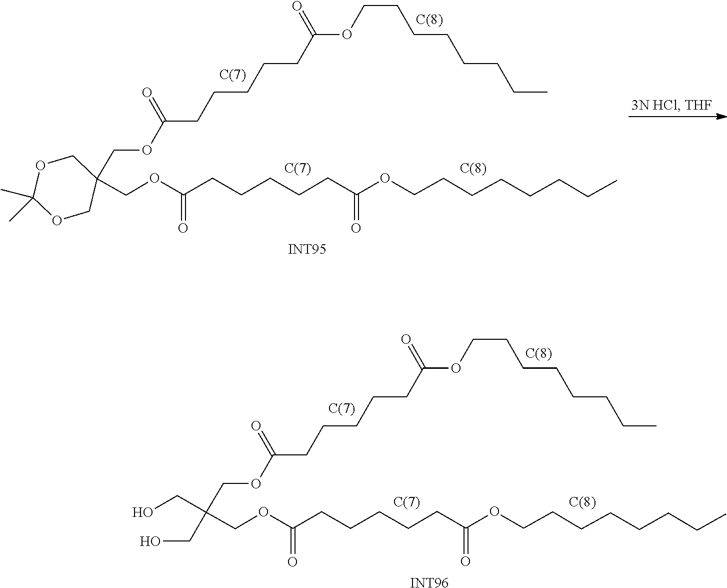

To a solution of O7-[[2,2-dimethyl-5-[(7-octoxy-7-oxo-heptanoyl)oxymethyl]-1,3-dioxan-5-yl]methyl]O1-octyl heptanedioate (7 g, 10.22 mmol, 1 eq) in THF (70 mL) was added HCl (3 M, 3.75 mL, 1.1 eq) at 0° C. under N₂ atmosphere. The reaction mixture was stirred at 25° C. for 12 hr under N₂ atmosphere. The reaction mixture was diluted with aq. NaHCO₃ (200 mL) and extracted with EtOAc 250 mL (50 mL*5). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2,2-bis(hydroxymethyl)-3-(7-octoxy-7-oxo-heptanoyl)oxy-propyl]O1-octyl heptanedioate (2.5 g, 3.88 mmol, 37.93% yield) was obtained as a colorless oil.

### Step 4:

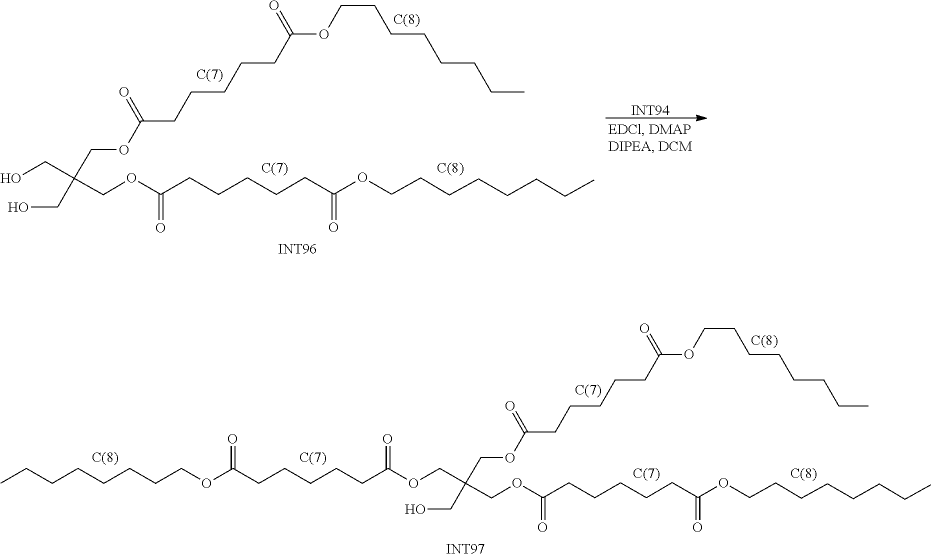

To a solution of O7-[2,2-bis(hydroxymethyl)-3-(7-octoxy-7-oxo-heptanoyl)oxy-propyl]O1-octyl heptanedioate (1 g, 1.55 mmol, 1 eq) in DCM (10 mL) was added EDCI (356.72 mg, 1.86 mmol, 1.2 eq), 7-octoxy-7-oxo-heptanoic acid (253.43 mg, 930.41 mol, 0.6 eq), DMAP (18.94 mg, 155.07 mol, 0.1 eq) and DIPEA (501.04 mg, 3.88 mmol, 675.26 L, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2-(hydroxymethyl)-3-(7-octoxy-7-oxo-heptanoyl)oxy-2-[(7-octoxy-7-oxo-heptanoyl)oxymethyl]propyl]O1-octyl heptanedioate (0.4 g, 444.82 mol, 28.69% yield) was obtained as a colorless oil. ¹H NMR (400 MHz, CDCl₃) δ ppm 4.16 (s, 6H), 4.06 (t, J=6.4 Hz, 6H), 3.52 (d, J=6.8 Hz, 2H), 2.66 (t, J=6.8 Hz, 1H), 2.36-2.29 (m, 12H), 1.69-1.59 (m, 19H), 1.40-1.27 (m, 34H), 0.89 (t, J=13.6 Hz, 9H).

### Step 5:

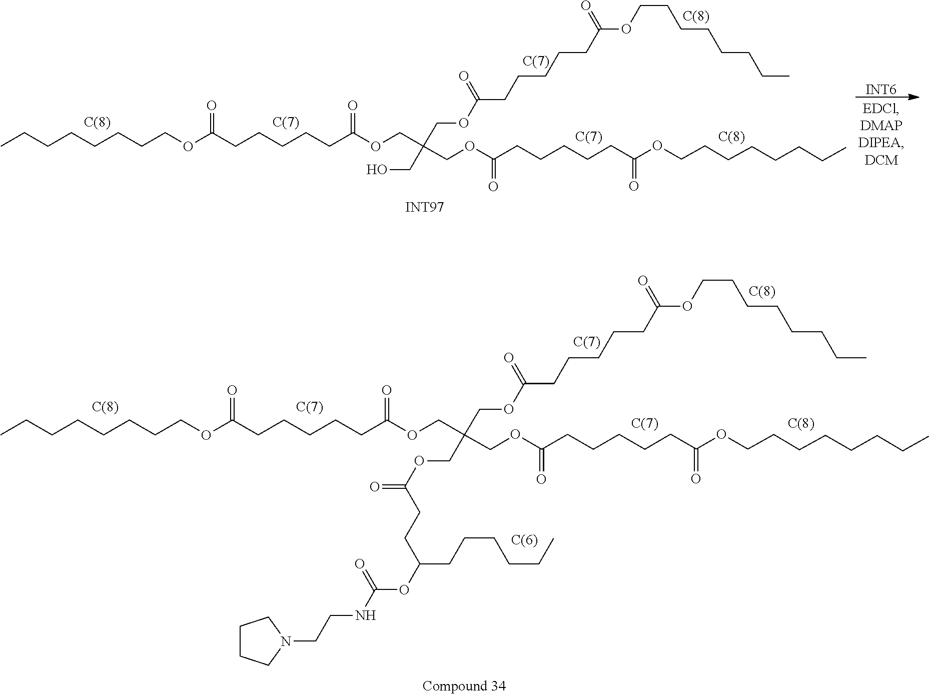

To a solution of O7-[2-(hydroxymethyl)-3-(7-octoxy-7-oxo-heptanoyl)oxy-2-[(7-octoxy-7-oxo-heptanoyl)oxymethyl]propyl]O1-octyl heptanedioate (0.4 g, 444.82 mol, 1 eq) in DCM (5 mL) was added EDCI (102.33 mg, 533.79 mol, 1.2 eq), 4-(2-pyrrolidin-1-ylethylcarbamoyloxy)decanoic acid (131.49 mg, 400.34 mol, 0.9 eq), DIPEA (143.72 mg, 1.11 mmol, 193.70 L, 2.5 eq) and DMAP (5.43 mg, 44.48 mol, 0.1 eq) under N₂ atmosphere. The reaction mixture was stirred at 25° C. for 12 hr under N₂ atmosphere. The reaction mixture was diluted with H₂O (50 mL) and extracted with DCM 50 mL (25 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O7-[2,2-bis[(7-octoxy-7-oxo-heptanoyl)oxymethyl]-3-[4-(2-pyrrolidin-1-ylethylcarbamoyloxy) decanoyloxy]propyl]O1-octyl heptanedioate was obtained as a colorless oil. ¹H NMR (400 MHz, CDCl₃) δ ppm 5.19 (s, 1H), 4.76 (d, J=2.0 Hz, 1H), 4.12 (s, 8H), 4.06 (t, J=6.8 Hz, 6H), 3.30 (d, J=5.2 Hz, 1H), 2.61 (t, J=12 Hz, 2H), 2.52 (s, 4H), 2.40-2.29 (m, 14H), 1.95-1.78 (m, 6H), 1.68-1.60 (m, 22H), 1.39-1.28 (m, 42H), 0.90-0.87 (m, 12H).

### Example 42—Compound 35

### O8-[2,2-bis[(8-decoxy-8-oxo-octanoyl)oxymethyl]-3-[4-(2-pyrrolidin-1ylethylcarbamoyloxy)decanoyloxy]propyl]O1-decyloctanedioate

### Step 1:

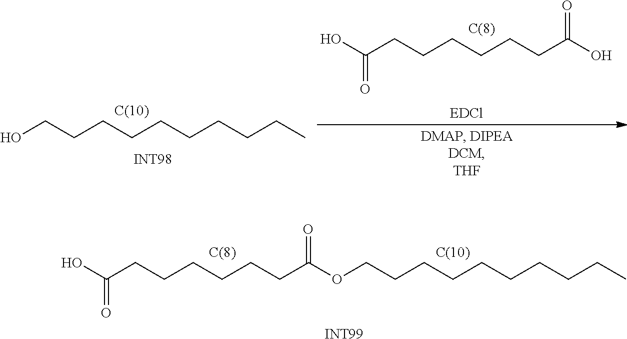

To a mixture of decan-1-ol (20 g, 126.36 mmol, 24.13 mL, 1 eq), DMAP (1.54 g, 12.64 mmol, 0.1 eq), EDCI (29.07 g, 151.63 mmol, 1.2 eq) and DIPEA (40.83 g, 315.90 mmol, 55.02 mL, 2.5 eq) in DCM (65 mL) and THF (65 mL) was added octanedioic acid (110.05 g, 631.79 mmol, 5 eq) under N₂ atmosphere. The reaction mixture was stirred at 25° C. for 12 hr under N₂ atmosphere. The reaction mixture was diluted with H₂O (600 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 8-decoxy-8-oxo-octanoic acid (25 g, 79.50 mmol, 62.92% yield) was obtained as a colorless oil. ¹H NMR (400 MHz, CDCl₃) δ ppm 11.71-10.55 (m, 1H), 4.06 (t, J=3.2 Hz, 2H), 2.37-2.28 (m, 4H), 1.69-1.58 (m, 6H), 1.43-1.27 (m, 18H), 0.89 (t, J=3.4 Hz, 3H).

### Step 2:

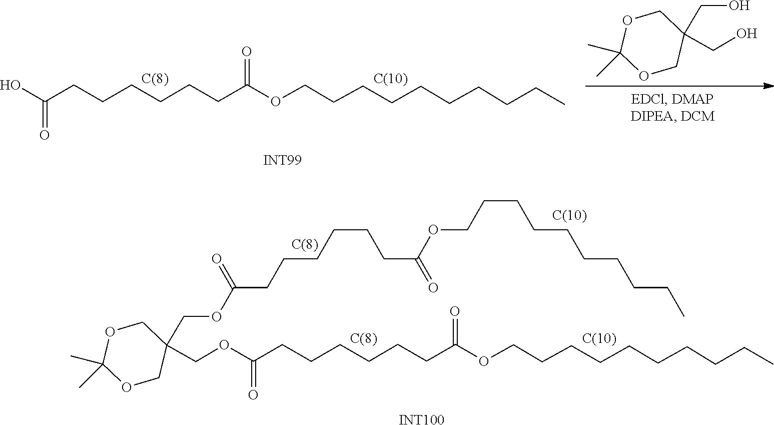

To a mixture of 8-decoxy-8-oxo-octanoic acid (7.93 g, 25.22 mmol, 1 eq), DIPEA (8.15 g, 63.06 mmol, 10.98 mL, 2.5 eq), EDCI (5.80 g, 30.27 mmol, 1.2 eq) and DMAP (616.27 mg, 5.04 mmol, 0.2 eq) in DCM (300 mL) was added [5-(hydroxymethyl)-2,2-dimethyl-1,3-dioxan-5-yl]methanol (2 g, 11.35 mmol, 0.45 eq) under N₂ atmosphere. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[[5-[(8-decoxy-8-oxo-octanoyl)oxymethyl]-2,2-dimethyl-1,3-dioxan-5-yl]methyl]O1-decyl octanedioate (4 g, 5.20 mmol, 20.62% yield) was obtained as a colorless oil. ¹H NMR (400 MHz, CDCl₃) δ ppm 4.10 (s, 4H), 4.05 (t, J=6.8 Hz, 4H), 3.74 (s, 4H), 2.30-2.90 (m, 8H), 1.63-1.60 (m, 12H), 1.42 (s, 6H), 1.22-1.36 (m, 36H), 0.88 (t, J=6.8 Hz, 6H).

### Step 3:

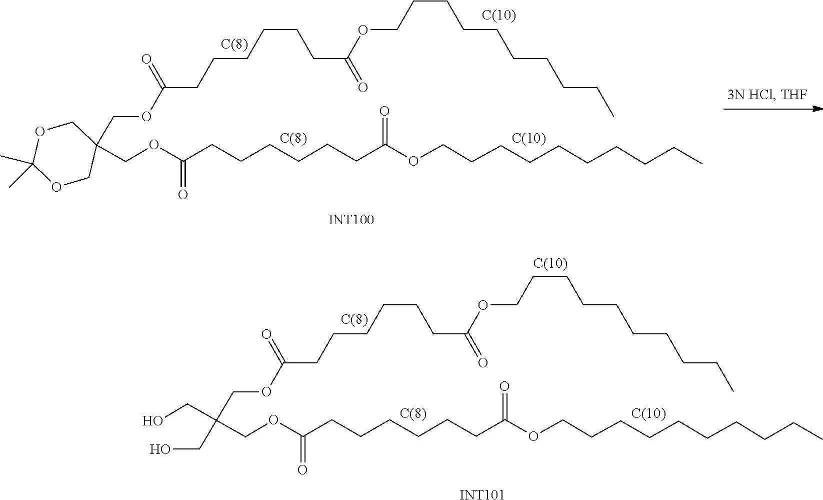

To a solution of O8-[[5-[(8-decoxy-8-oxo-octanoyl)oxymethyl]-2,2-dimethyl-1,3-dioxan-5-yl]methyl]O1-decyl octanedioate (4 g, 5.20 mmol, 1 eq) in THF (40 mL) was added HCl (2 M, 2.86 mL, 1.1 eq) at 0° C. under N₂ atmosphere. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2-[(8-decoxy-8-oxo-octanoyl)oxymethyl]-3-hydroxy-2-(hydroxymethyl)propyl]O1-decyl octanedioate (1.5 g, 2.06 mmol, 39.56% yield) was obtained as a colorless oil.

### Step 4:

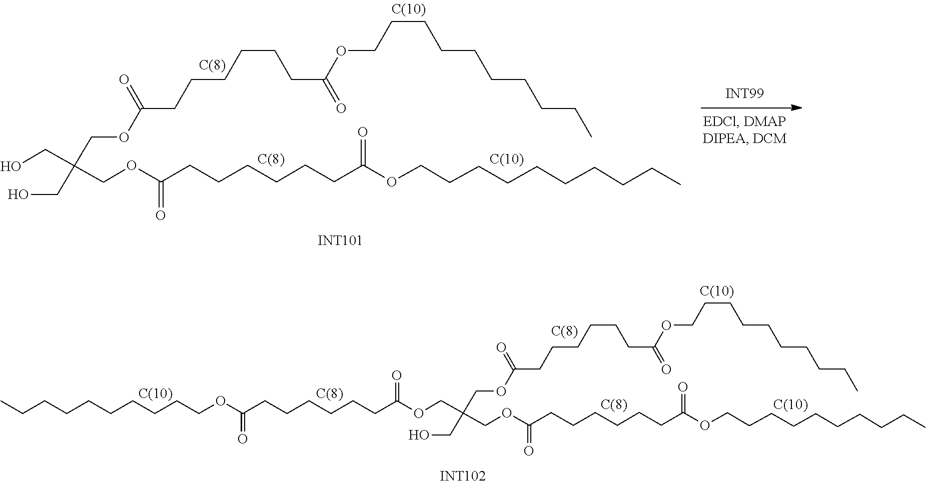

To a mixture of O8-[2-[(8-decoxy-8-oxo-octanoyl)oxymethyl]-3-hydroxy-2-(hydroxymethyl)propyl]O1-decyl octanedioate (1.5 g, 2.06 mmol, 1 eq), EDCI (473.32 mg, 2.47 mmol, 1.2 eq), DMAP (25.14 mg, 205.75 mol, 0.1 eq) and DIPEA (664.80 mg, 5.14 mmol, 895.96 μL, 2.5 eq) in DCM (15 mL) was added 8-decoxy-8-oxo-octanoic acid (388.20 mg, 1.23 mmol, 0.6 eq) under N₂ atmosphere. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis[(8-decoxy-8-oxo-octanoyl)oxymethyl]-3-hydroxy-propyl]O1-decyl octanedioate (800 mg, 780.12 mol, 37.92% yield) was obtained as colorless oil. ¹H NMR (400 MHz, CDCl₃) δ ppm 4.11 (s, 6H), 4.05 (t, J=6.8 Hz, 6H), 3.50 (d, J=6.8 Hz, 1H), 3.56-3.43 (m, 1H), 2.61 (t, J=6.6 Hz, 1H), 2.37-2.27 (m, 12H), 1.67-1.58 (m, 18H), 1.37-1.20 (m, 54H), 0.97-0.73 (m, 9H).

### Step 5:

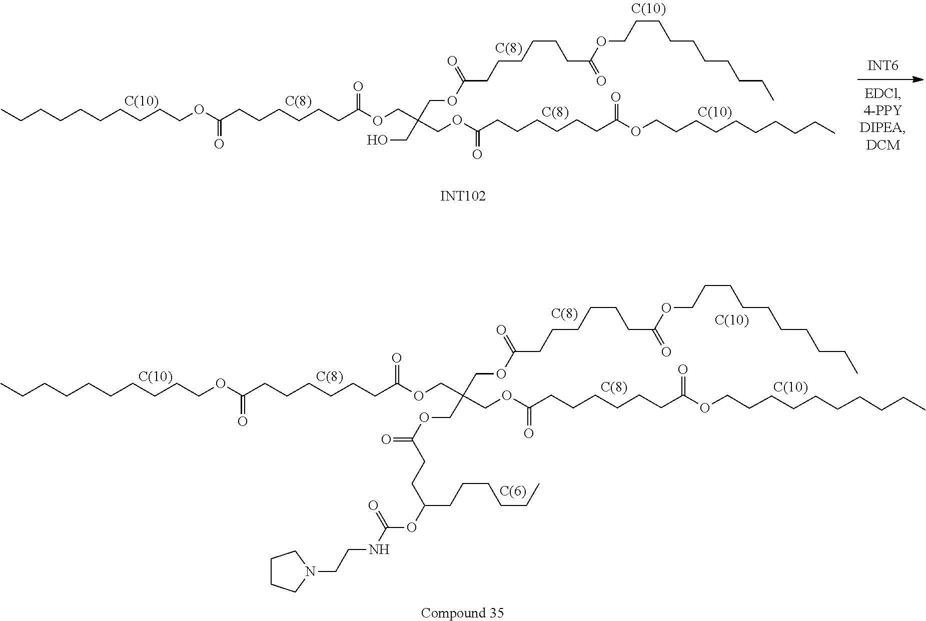

To a solution of O8-[2,2-bis[(8-decoxy-8-oxo-octanoyl)oxymethyl]-3-hydroxy-propyl]O1-decyloctanedioate (1.5 g, 1.46 mmol, 1 eq) in DCM (20 mL) was added 4-(2-pyrrolidin-1-ylethylcarbamoyloxy)decanoic acid (576.51 mg, 1.76 mmol, 1.2 eq), DIPEA (472.62 mg, 3.66 mmol, 636.95 μL, 2.5 eq), 4-pyrrolidin-1-ylpyridine (216.78 mg, 1.46 mmol, 1 eq) and EDCI (336.49 mg, 1.76 mmol, 1.2 eq) under N₂ atmosphere. The reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis[(8-decoxy-8-oxo-octanoyl)oxymethyl]-3-[4-(2-pyrrolidin-1ylethylcarbamoyloxy)decanoyloxy]propyl]O1-decyloctanedioate (110 mg, 82.34 mol, 5.63% yield, 100% purity) was obtained as a colorless oil. ¹H NMR (400 MHz, CDCl₃) δ ppm 5.18 (br s, 1H), 4.75 (br s, 1H), 4.14-4.09 (m, 8H), 4.06 (t, J=6.8 Hz, 6H), 3.29 (d, J=5.2 Hz, 2H), 2.59 (t, J=5.9 Hz, 2H), 2.52 (br s, 4H), 2.40-2.34 (m, 2H), 2.33-2.27 (m, 12H), 1.94-1.84 (m, 1H), 1.78 (br s, 4H), 1.61 (m, 18H), 1.44-1.11 (m, 66H), 0.96-0.81 (m, 12H).

### Example 43—Compound 36

### O7-[2,2-bis[(7-decoxy-7-oxo-heptanoyl)oxymethyl]-3-[4-(2-pyrrolidin-1-ylethylcarbamoyloxy)decanoyloxy]propyl]O1-decyl heptanedioate

### Step 1:

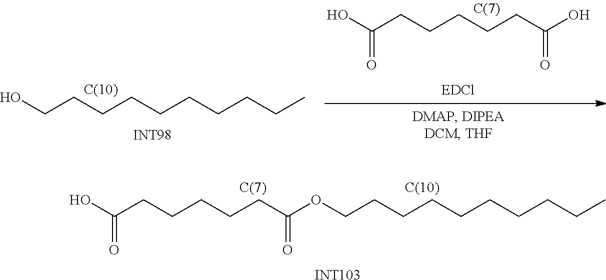

To a mixture of decan-1-ol (20 g, 126.36 mmol, 24.13 mL, 1 eq), EDCI (29.07 g, 151.63 mmol, 1.2 eq), DMAP (1.54 g, 12.64 mmol, 0.1 eq) and DIPEA (40.83 g, 315.90 mmol, 55.02 mL, 2.5 eq) in DCM (600 mL) and THF (600 mL) was added heptanedioic acid (101.19 g, 631.79 mmol, 5 eq) under N₂ atmosphere. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 7-decoxy-7-oxo-heptanoic acid (30 g, 99.86 mmol, 79.03% yield) was obtained as a colorless oil. ¹H NMR (400 MHz, CDCl₃) δ ppm 11.27 (br s, 1H), 4.06 (t, J=6.8 Hz, 2H), 2.45-2.29 (m, 4H), 1.71-1.56 (m, 6H), 1.45-1.24 (m, 16H), 0.96-0.83 (m, 3H).

### Step 2:

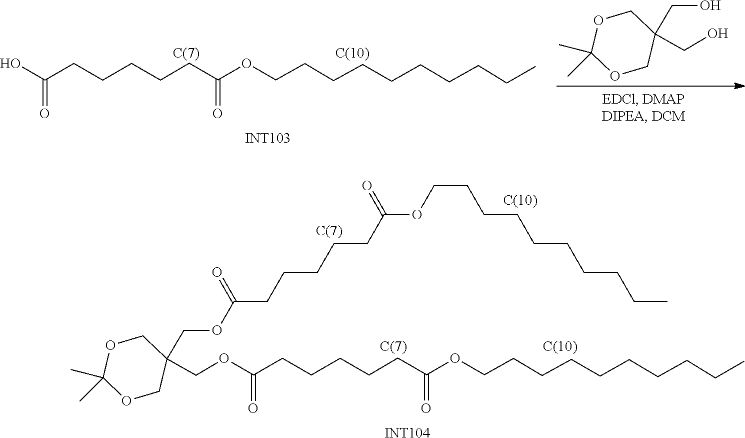

To a mixture of [5-(hydroxymethyl)-2,2-dimethyl-1,3-dioxan-5-yl]methanol (3 g, 17.03 mmol, 0.45 eq), DMAP (462.20 mg, 3.78 mmol, 0.1 eq), EDCI (8.70 g, 45.40 mmol, 1.2 eq) and DIPEA (12.22 g, 94.58 mmol, 16.47 mL, 2.5 eq) in DCM (150 mL) was added 7-decoxy-7-oxo-heptanoic acid (11.37 g, 37.83 mmol, 1 eq) under N₂ atmosphere. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[[5-[(7-decoxy-7-oxo-heptanoyl)oxymethyl]-2,2-dimethyl-1,3-dioxan-5-yl]methyl]O1-decyl heptanedioate (3.5 g, 4.72 mmol, 12.48% yield) was obtained as a colorless oil. ¹H NMR (400 MHz, CDCl₃) δ ppm 4.11 (s, 4H), 4.06 (t, J=6.8 Hz, 4H), 3.74 (s, 4H), 2.32 (m, 8H), 1.73-1.59 (m, 12H), 1.42 (s, 6H), 1.39-1.23 (m, 32H), 0.90-0.86 (m, 6H).

### Step 3:

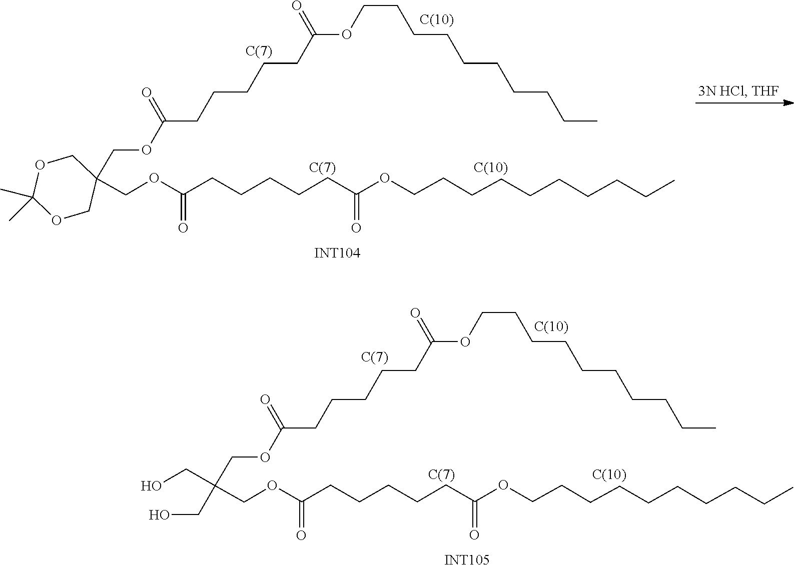

To a mixture of O7-[[5-[(7-decoxy-7-oxo-heptanoyl)oxymethyl]-2,2-dimethyl-1,3-dioxan-5-yl]methyl]O1-decyl heptanedioate (3.5 g, 4.72 mmol, 1 eq) in THF (35 mL) was added HCl (3 M, 1.73 mL, 1.1 eq) at 0° C. under N₂ atmosphere. The reaction mixture was stirred at 25° C. for 12 hr under N₂ atmosphere. The reaction mixture was diluted with aq. NaHCO₃ (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2-[(7-decoxy-7-oxo-heptanoyl)oxymethyl]-3-hydroxy-2-(hydroxymethyl)propyl]O1-decyl heptanedioate (1.2 g, 1.71 mmol, 36.25% yield) was obtained as a colorless oil. ¹H NMR (400 MHz, CDCl₃) δ ppm 4.15 (s, 4H), 4.06 (t, J=6.8 Hz, 4H), 3.59 (br d, J=4.2 Hz, 4H), 2.78 (br s, 2H), 2.45-2.25 (m, 8H), 1.69-1.59 (m, 12H), 1.40-1.24 (m, 32H), 0.97-0.82 (m, 6H).

### Step 4:

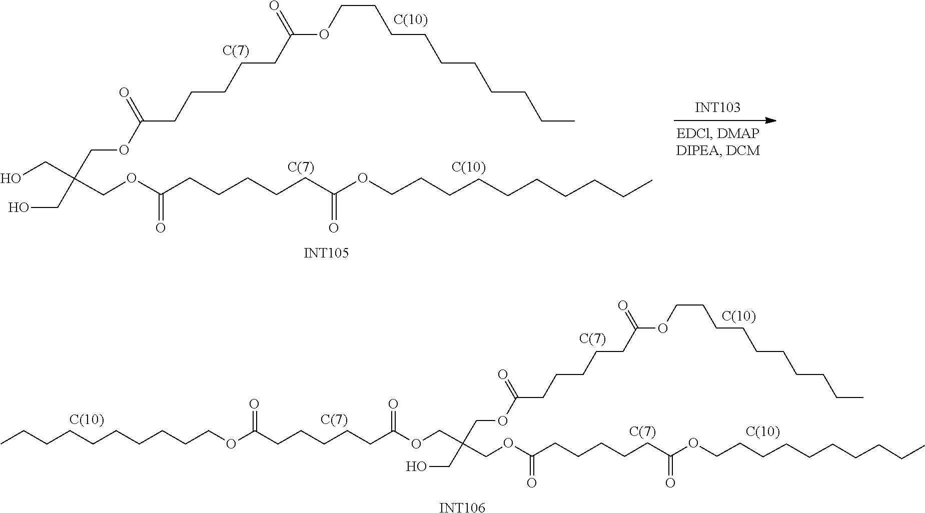

To a mixture of O7-[2-[(7-decoxy-7-oxo-heptanoyl)oxymethyl]-3-hydroxy-2-(hydroxymethyl)propyl]O1-decyl heptanedioate (1 g, 1.43 mmol, 1 eq), DIPEA (460.94 mg, 3.57 mmol, 621.21 μL, 2.5 eq), EDCI (328.17 mg, 1.71 mmol, 1.2 eq) and DMAP (17.43 mg, 142.66 mol, 0.1 eq) in DCM (15 mL) was added 7-decoxy-7-oxo-heptanoic acid (257.15 mg, 855.94 mol, 0.6 eq) under N₂ atmosphere. The reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[(7-decoxy-7-oxo-heptanoyl)oxymethyl]-3-hydroxy-propyl]O1-decyl heptanedioate (0.4 g, 406.75 mol, 28.51% yield) was obtained as a colorless oil. ¹H NMR (400 MHz, CDCl₃) δ ppm 4.11 (s, 6H), 4.05 (t, J=6.8 Hz, 6H), 3.51 (br d, J=6.4 Hz, 2H), 2.68 (t, J=6.6 Hz, 1H), 2.32 (m, 12H), 1.68-1.59 (m, 18H), 1.40-1.24 (m, 48H), 0.88 (t, J=6.6 Hz, 9H)

### Step 5:

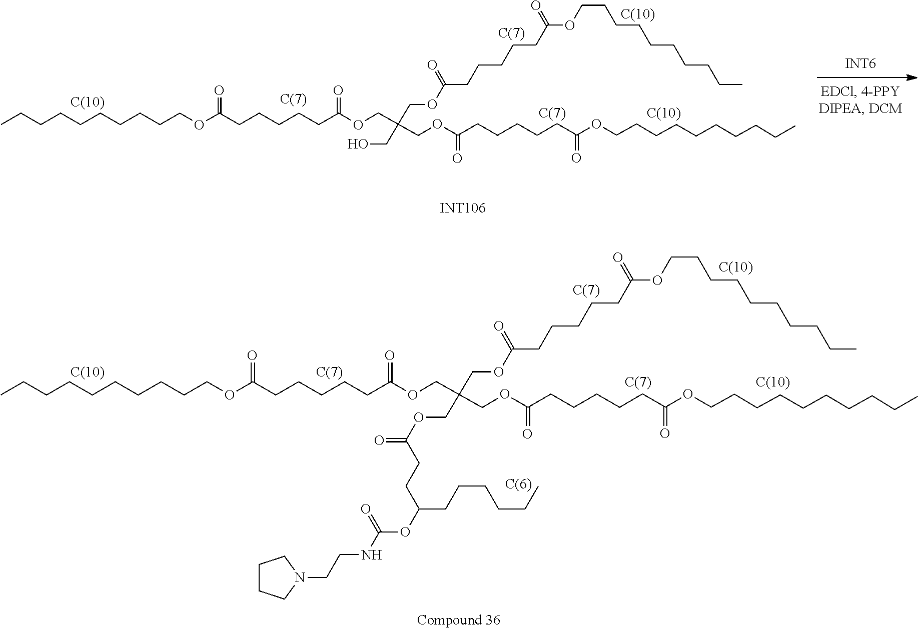

To a mixture of O7-[2,2-bis[(7-decoxy-7-oxo-heptanoyl)oxymethyl]-3-hydroxy-propyl]O1-decylheptanedioate (0.7 g, 711.82 mol, 1 eq), DIPEA (229.99 mg, 1.78 mmol, 309.96 μL, 2.5 eq), EDCI (163.75 mg, 854.18 mol, 1.2 eq) and 4-pyrrolidin-1-ylpyridine (10.55 mg, 71.18 mol, 0.1 eq) in DCM (10 mL) was added 4-(2-pyrrolidin-1-ylethylcarbamoyloxy)decanoic acid (280.55 mg, 854.18 mol, 1.2 eq) under N₂ atmosphere. The reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[(7-decoxy-7-oxo-heptanoyl)oxymethyl]-3-[4-(2-pyrrolidin-1-ylethylcarbamoyloxy)decanoyloxy]propyl]O1-decyl heptanedioate (130 mg, 100.48 mol, 14.12% yield, 100% purity) was obtained as a colorless oil. ¹H NMR (400 MHz, CDCl₃) δ ppm 5.21 (br s, 1H), 4.75 (br s, 1H), 4.11 (s, 8H), 4.06 (t, J=6.8 Hz, 6H), 3.30 (br d, J=4.8 Hz, 2H), 2.60 (br s, 2H), 2.53 (br s, 4H), 2.42-2.36 (m, 2H), 2.31 (d, J=7.4 Hz, 10H), 1.90 (m, 1H), 1.78 (br s, 6H), 1.71-1.59 (m, 20H), 1.46-1.24 (m, 58H), 0.98-0.82 (m, 12H).

### Example 44—Compound 37

### O1-[2,2-bis[[8-(1-methyloctoxy)-8-oxo-octanoyl]oxymethyl]-3-[4-(2-pyrrolidin-1-ylethylcarbamoyloxy) decanoyloxy]propyl]O8-(1-methyloctyl) octanedioate

### Step 1:

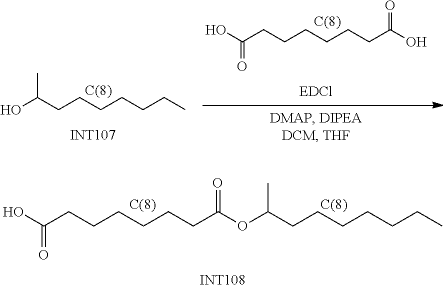

To a solution of octanedioic acid (60.38 g, 346.61 mmol, 5 eq) in DCM 600 mL was added EDCI (15.95 g, 83.19 mmol, 1.2 eq), DMAP (846.89 mg, 6.93 mmol, 0.1 eq) and DIPEA (22.40 g, 173.31 mmol, 30.19 mL, 2.5 eq) under N₂ atmosphere. Then nonan-2-ol (10 g, 69.32 mmol, 1 eq) in DCM (100 ml) was added to the above reaction mixture and the reaction mixture was stirred at 25° C. for 12 hr under N₂ atmosphere. The reaction mixture was diluted with H₂O (500 mL) and extracted with DCM 150 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound 8-(1-methyloctoxy)-8-oxo-octanoic acid (6 g, 19.97 mmol, 28.81% yield) was obtained as a colorless oil. ¹H NMR (400 MHz, CDCl₃) δ ppm 10.42 (br s, 1H), 4.80-5.00 (m, 1H), 2.36 (t, J=7.4 Hz, 2H), 2.28 (t, J=7.6 Hz, 2H), 1.71-1.41 (m, 5H), 1.49-1.41 (m, 1H), 1.40-1.33 (m, 4H), 1.33-1.22 (m, 10H), 1.20 (d, J=6.2 Hz, 3H), 0.96-0.80 (m, 3H).

### Step 2:

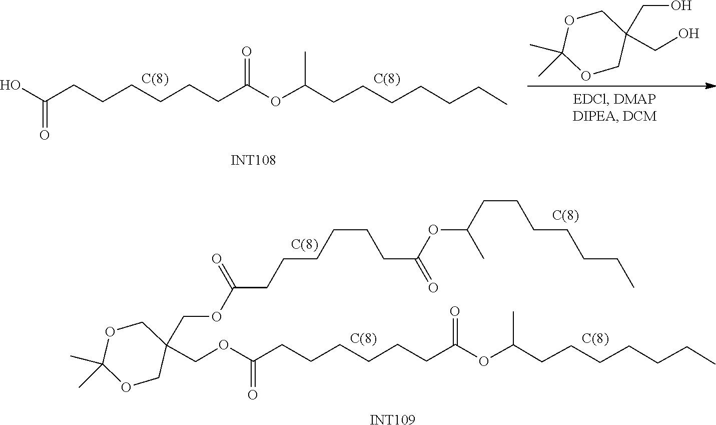

To a solution of 8-(1-methyloctoxy)-8-oxo-octanoic acid (15.16 g, 50.44 mmol, 1 eq) in DCM (200 mL) was added EDCI (11.60 g, 60.53 mmol, 1.2 eq), [5-(hydroxymethyl)-2,2-dimethyl-1,3-dioxan-5-yl]methanol (4 g, 22.70 mmol, 0.45 eq), DMAP (616.27 mg, 5.04 mmol, 0.1 eq) and DIPEA (16.30 g, 126.11 mmol, 21.97 mL, 2.5 eq) under N₂ atmosphere. The reaction mixture was stirred at 25° C. for 12 hr under N₂ atmosphere. The reaction mixture was diluted with H₂O (200 mL) and extracted with DCM 800 mL (200 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[[2,2-dimethyl-5-[[8-(1-methyloctoxy)-8-oxo-octanoyl]oxymethyl]-1,3-dioxan-5-yl]methyl]O8-(1-methyloctyl) octanedioate (8 g, 10.80 mmol, 21.40% yield) was obtained as a colorless oil. ¹H NMR (400 MHz, CDCl₃) δ ppm 4.92-4.87 (m, 2H), 4.11 (s, 4H), 3.74 (s, 4H), 2.30 (m, 8H), 1.71-1.56 (m, 10H), 1.48 (m, 2H), 1.42 (s, 6H), 1.39-1.31 (m, 10H), 1.31-1.25 (m, 18H), 1.20 (d, J=6.2 Hz, 6H), 0.93-0.83 (m, 6H).

### Step 3:

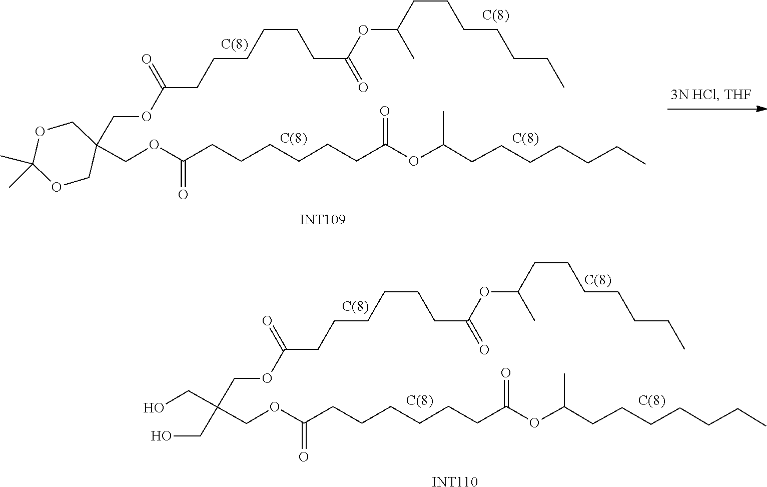

To a solution of O1-[[2,2-dimethyl-5-[[8-(1-methyloctoxy)-8-oxo-octanoyl]oxymethyl]-1,3-dioxan-5-yl]methyl]O8-(1-methyloctyl) octanedioate (7 g, 9.45 mmol, 1 eq) in THF (70 mL) was added HCl (3 M, 3.46 mL, 1.1 eq) at 0° C. under N₂ atmosphere. The reaction mixture was stirred at 25° C. for 2 hr under N₂ atmosphere. The reaction mixture was diluted with aq. NaHCO₃ (200 mL) and extracted with EtOAc 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis(hydroxymethyl)-3-[8-(1-methyloctoxy)-8-oxo-octanoyl]oxy-propyl]O8-(1-methyloctyl)octanedioate (4 g, 5.71 mmol, 60.41% yield) was obtained as a colorless oil. ¹H NMR (400 MHz, CDCl₃) δ ppm 4.92-4.88 (m, 2H), 4.15 (s, 4H), 3.59 (s, 4H), 2.78 (br s, 2 H), 2.36 (t, J=7.6 Hz, 4H), 2.28 (t, J=7.4 Hz, 4H), 1.63 (m, 10H), 1.52-1.42 (m, 2H), 1.37-1.23 (m, 28H), 1.20 (d, J=6.2 Hz, 6H), 0.96-0.80 (m, 6H).

### Step 4:

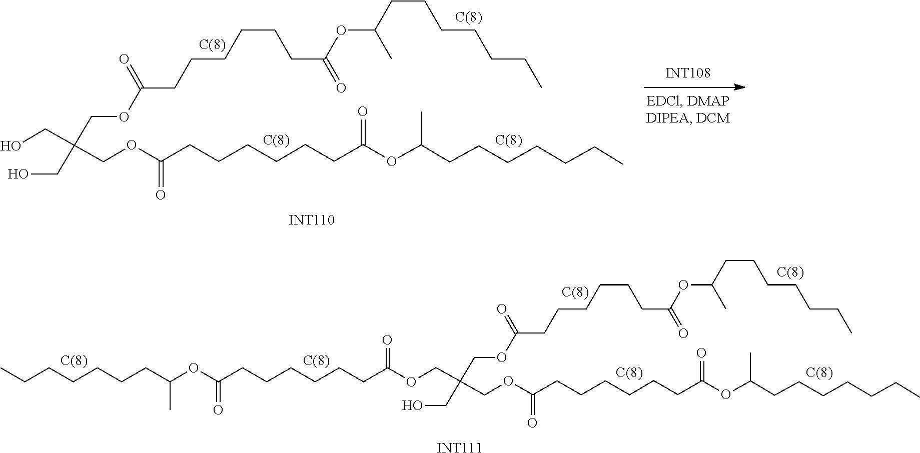

To a solution of O1-[2,2-bis(hydroxymethyl)-3-[8-(1-methyloctoxy)-8-oxo-octanoyl]oxy-propyl]O8-(1-methyloctyl) octanedioate (3 g, 4.28 mmol, 1 eq) in DCM (37 mL) was added EDCI (984.51 mg, 5.14 mmol, 1.2 eq), 8-(1-methyloctoxy)-8-oxo-octanoic acid (771.46 mg, 2.57 mmol, 0.6 eq), DMAP (52.28 mg, 427.97 mol, 0.1 eq) and DIPEA (1.38 g, 10.70 mmol, 1.86 mL, 2.5 eq) under N₂ atmosphere. The reaction mixture was stirred at 25° C. for 12 hr under N₂ atmosphere. The reaction mixture was diluted with H₂O (100 mL) and extracted with DCM 400 ml (100 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O1-[2-(hydroxymethyl)-3-[8-(1-methyloctoxy)-8-oxo-octanoyl]oxy-2-[[8-(1-methyloctoxy)-8-oxo-octanoyl]oxymethyl]propyl]O8-(1-methyloctyl) octanedioate (1.5 g, 1.53 mmol, 35.64% yield) was obtained as a colorless oil. ¹H NMR (400 MHz, CDCl₃) δ ppm 4.92-4.86 (m, 3H), 4.12 (s, 6H), 3.51 (d, J=9.6 Hz, 2H), 2.61 (t, J=6.8 Hz, 1H), 2.35-2.26 (m, 12H), 1.64-1.61 (m, 12H), 1.49-1.43 (m, 4H), 1.36-1.32 (m, 14H), 1.30-1.28 (m, 30H), 1.20 (t, J=6.4 Hz, 9H), 0.88 (t, J=13.6 Hz, 9H).

### Step 5:

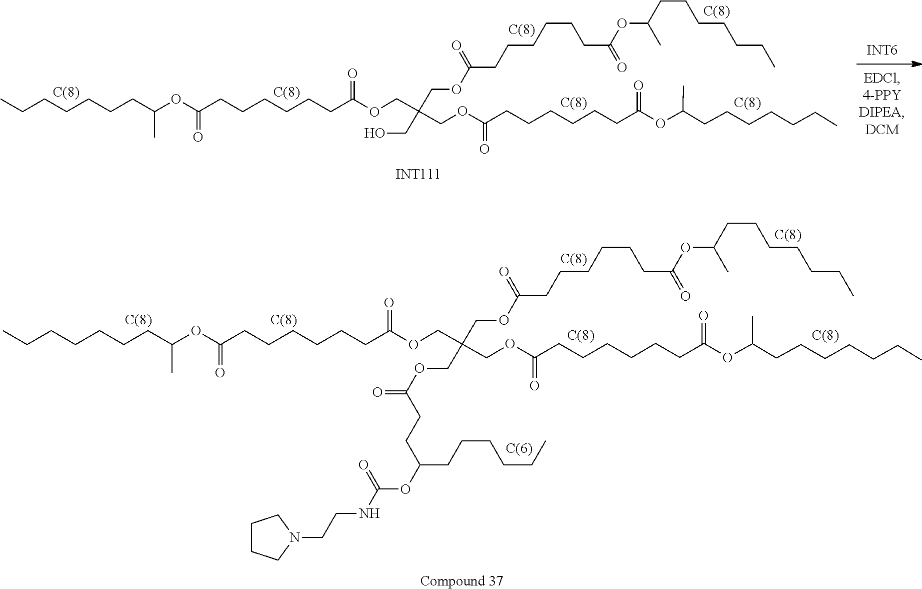

To a solution of O1-[2-(hydroxymethyl)-3-[8-(1-methyloctoxy)-8-oxo-octanoyl]oxy-2-[[8-(1-methyloctoxy)-8-oxo-octanoyl]oxymethyl]propyl]O8-(1-methyloctyl) octanedioate (1.8 g, 1.83 mmol, 1 eq) in DCM (19 mL) was added EDCI (421.07 mg, 2.20 mmol, 1.2 eq), 4-(2-pyrrolidin-1-ylethylcarbamoyloxy)decanoic acid (541.07 mg, 1.65 mmol, 0.9 eq), 4-pyrrolidin-1-ylpyridine (27.13 mg, 183.04 μmol, 0.1 eq) and DIPEA (591.41 mg, 4.58 mmol, 797.05 L, 2.5 eq) under N₂ atmosphere. The reaction mixture was stirred at 25° C. for 12 hr under N₂ atmosphere. The reaction mixture was diluted with H₂O (100 mL) and extracted with DCM 400 mL (100 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis[[8-(1-methyloctoxy)-8-oxo-octanoyl]oxymethyl]-3-[4-(2-pyrrolidin-1-ylethylcarbamoyloxy) decanoyloxy]propyl]O8-(1-methyloctyl) octanedioate (0.1 g, 61.83 mol, 3.38% yield, 80% purity) was obtained as a colorless oil. ¹H NMR (400 MHz, CDCl₃) δ ppm 5.40-5.35 (m, 1H), 4.94-4.74 (m, 3H), 4.75 (s, 1H), 4.11-4.09 (s, 8H), 3.37 (s, 2H), 2.72-2.60 (s, 6H), 2.40-2.25 (m, 14H), 1.86-1.77 (m, 6H), 1.64-1.55 (m, 18H), 1.49-1.45 (m, 4H), 1.35-1.27 (m, 48H), 1.20 (d, J=6.4 Hz, 9H), 0.88 (t, J=13.6 Hz, 12H).

### Example 45—Compound 38

### O1-[2,2-bis[[7-(1-methyloctoxy)-7-oxo-heptanoyl]oxymethyl]-3-[4-(2-pyrrolidin-1-ylethylcarbamoyloxy) decanoyloxy]propyl]O7-(1-methyloctyl) heptanedioate

### Step 1:

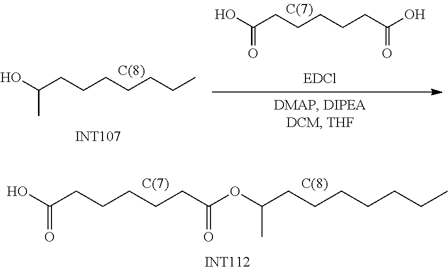

To a solution of nonan-2-ol (20 g, 138.64 mmol, 1 eq) in DCM (1310 mL) was added EDCI (34.55 g, 180.24 mmol, 1.3 eq), heptanedioic acid (111.03 g, 693.22 mmol, 5 eq), DMAP (1.69 g, 13.86 mmol, 0.1 eq) and DIPEA (44.80 g, 346.61 mmol, 60.37 mL, 2.5 eq) under N₂ atmosphere. The reaction mixture was stirred at 25° C. for 12 hr under N₂ atmosphere. The reaction mixture was diluted with H₂O (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound 7-(1-methyloctoxy)-7-oxo-heptanoic acid (30 g, 104.75 mmol, 75.55% yield) was obtained as a colorless oil. ¹H NMR (400 MHz, CDCl₃) δ ppm 4.93-4.88 (m, 1H), 2.37 (t, J=7.6 Hz, 2H), 2.29 (t, J=7.6 Hz, 2H), 1.74-1.61 (m, 4H), 1.61-1.52 (m, 1H), 1.52-1.34 (m, 3H), 1.33-1.23 (m, 10H), 1.20 (d, J=6.2 Hz, 3H), 0.96-0.82 (m, 3H).

### Step 2:

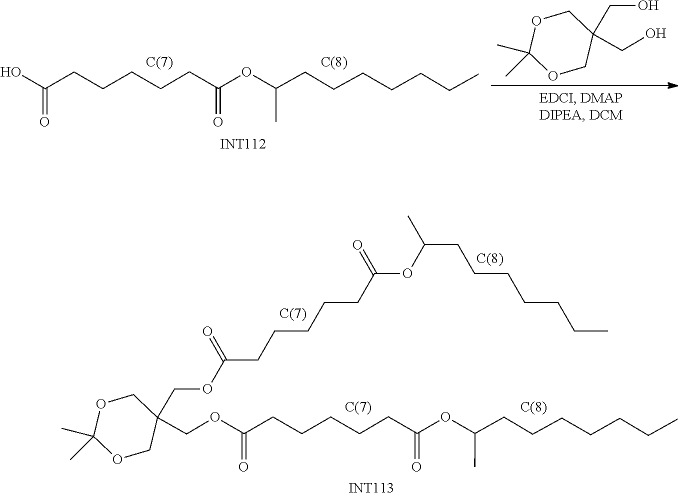

To a solution of 7-(1-methyloctoxy)-7-oxo-heptanoic acid (3.61 g, 12.61 mmol, 1 eq) in DCM (46 mL) was added EDCI (2.90 g, 15.13 mmol, 1.2 eq), [5-(hydroxymethyl)-2,2-dimethyl-1,3-dioxan-5-yl]methanol (1 g, 5.68 mmol, 0.45 eq), DMAP (154.07 mg, 1.26 mmol, 0.1 eq) and DIPEA (4.07 g, 31.53 mmol, 5.49 mL, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[[2,2-dimethyl-5-[[7-(1-methyloctoxy)-7-oxo-heptanoyl]oxymethyl]-1,3-dioxan-5-yl]methyl]O7-(1-methyloctyl)heptanedioate (1.5 g, 2.10 mmol, 16.68% yield) was obtained as a colorless oil. ¹H NMR (400 MHz, CDCl₃) δ ppm 4.92-4.88 (m, 2H), 4.11 (s, 4H), 3.75 (s, 4H), 2.37-2.13 (m, 8H), 1.69-1.60 (m, 8H), 1.47 (br d, J=8.0 Hz, 2H), 1.56 (s, 2H), 1.42 (s, 6H), 1.39-1.22 (m, 24H), 1.20 (d, J=6.2 Hz, 6H), 0.98-0.71 (m, 6H).

### Step 3:

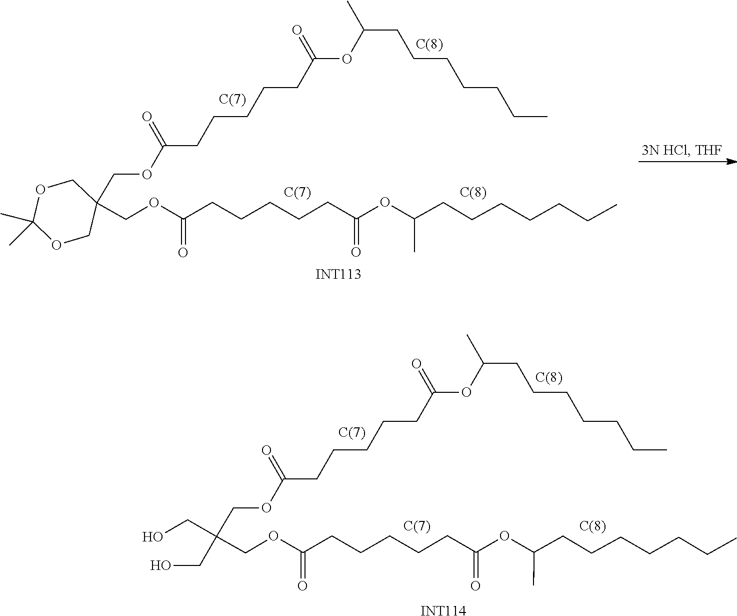

To a solution of O1-[[2,2-dimethyl-5-[[7-(1-methyloctoxy)-7-oxo-heptanoyl]oxymethyl]-1,3-dioxan-5-yl]methyl]O7-(1-methyloctyl) heptanedioate (1.5 g, 2.10 mmol, 1 eq) in THF (15 mL) was added HCl (3 M, 771.40 L, 1.1 eq) at 0° C. under N₂ atmosphere. The reaction mixture was stirred at 25° C. for 2 hr under N₂ atmosphere. The reaction mixture was diluted with aq. NaHCO₃ (50 mL) and extracted with EtOAC 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis(hydroxymethyl)-3-[7-(1-methyloctoxy)-7-oxo-heptanoyl]oxy-propyl]O7-(1-methyloctyl)heptanedioate (0.6 g, 891.63 mol, 42.38% yield) was obtained as a colorless oil. ¹H NMR (400 MHz, CDCl₃) δ ppm 4.92-4.87 (m, 2H), 4.15 (s, 4H), 3.59 (s, 4H), 2.37 (t, J= 7.2 Hz, 4H), 2.29 (t, J=7.6 Hz, 4H), 1.70-1.55 (m, 12H), 1.50-1.32 (m, 8H), 1.28 (s, 18H) 1.20 (d, J=6.0 Hz, 6H), 0.89 (t, J=6.4 Hz, 6H).

### Step 4:

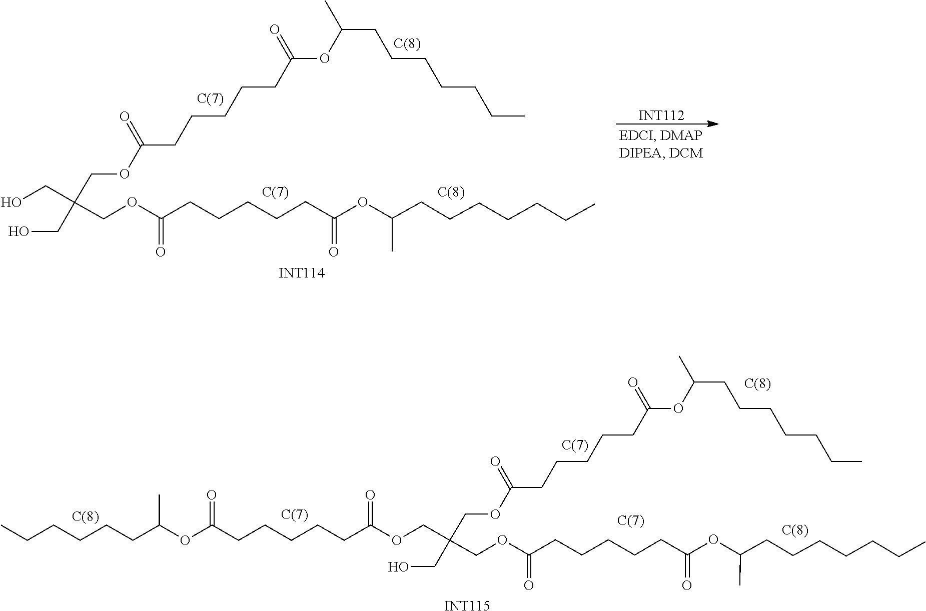

To a solution of O1-[2,2-bis(hydroxymethyl)-3-[7-(1-methyloctoxy)-7-oxo-heptanoyl]oxy-propyl]O7-(1-methyloctyl) heptanedioate (0.6 g, 891.63 mol, 1 eq) in DCM (1 mL) was added EDCI (205.11 mg, 1.07 mmol, 1.2 eq), 7-(1-methyloctoxy)-7-oxo-heptanoic acid (153.22 mg, 534.98 mol, 0.6 eq), DMAP (10.89 mg, 89.16 mol, 0.1 eq) and DIPEA (288.09 mg, 2.23 mmol, 388.26 L, 2.5 eq) under N₂ atmosphere. The reaction mixture was stirred at 25° C. for 12 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O1-[2-(hydroxymethyl)-3-[7-(1-methyloctoxy)-7-oxo-heptanoyl]oxy-2-[[7-(1-methyloctoxy)-7-oxo-heptanoyl]oxymethyl]propyl]O7-(1-methyloctyl) heptanedioate (0.3 g, 318.70 mol, 35.74% yield) was obtained as a colorless oil. ¹H NMR (400 MHz, CDCl₃) δ ppm 4.92-4.87 (m, 3H), 4.11 (s, 6H), 3.51 (d, J=6.4 Hz, 2H), 2.69 (t, J=13.6 Hz, 1H), 2.36-2.26 (m, 12H), 1.69-1.62 (m, 12H), 1.58-1.44 (m, 6H), 1.40-1.32 (m, 8H), 1.27 (s, 28H), 1.20 (d, J=6 Hz, 9H), 0.88 (t, J=13.6 Hz, 9H).

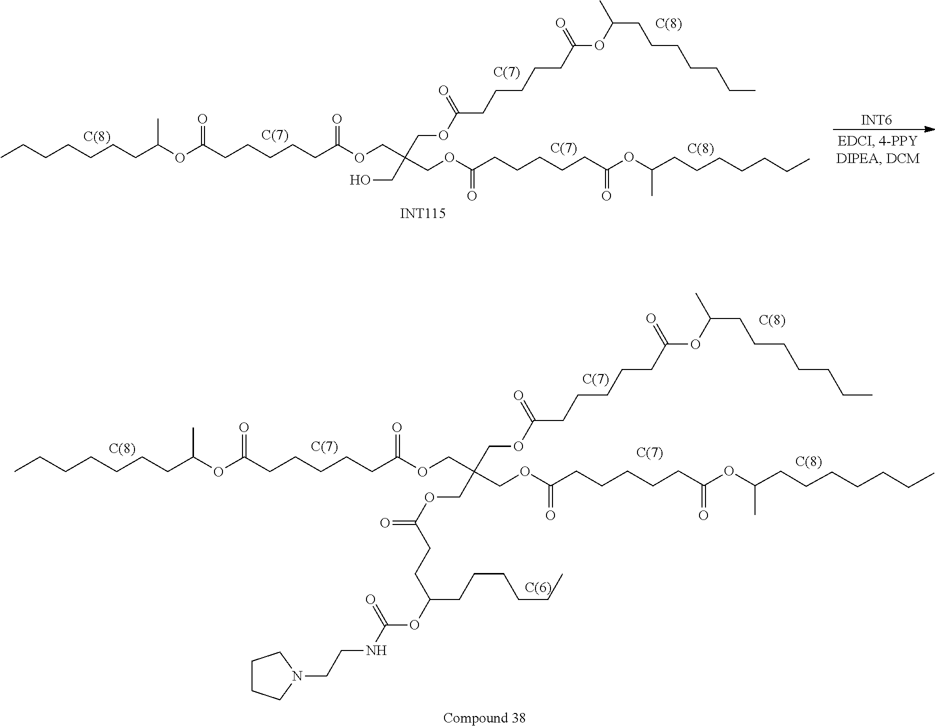

To a solution of O1-[2-(hydroxymethyl)-3-[7-(1-methyloctoxy)-7-oxo-heptanoyl]oxy-2-[[7-(1-methyloctoxy)-7-oxo-heptanoyl]oxymethyl]propyl]O7-(1-methyloctyl) heptanedioate (0.9 g, 956.11 mol, 1 eq) in DCM (10 mL) was added EDCI (219.94 mg, 1.15 mmol, 1.2 eq), 4-(2-pyrrolidin-1-ylethylcarbamoyloxy)decanoic acid (282.63 mg, 860.50 mol, 0.9 eq), 4-pyrrolidin-1-ylpyridine (14.17 mg, 95.61 mol, 0.1 eq) and DIPEA (308.93 mg, 2.39 mmol, 416.34 L, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis[[7-(1-methyloctoxy)-7-oxo-heptanoyl]oxymethyl]-3-[4-(2-pyrrolidin-1-ylethylcarbamoyloxy) decanoyloxy]propyl]O7-(1-methyloctyl) heptanedioate (0.1 g, 79.82 mol, 8.35% yield, 99.92% purity) was obtained as a colorless oil. ¹H NMR (400 MHz, CDCl₃) δ ppm 5.35-5.32 (s, 1H), 4.92-4.87 (m, 3H), 4.75 (s, 1H), 4.11 (s, 8H), 3.34 (d, J=4.0 Hz, 2H), 2.65 (d, J=18.4 Hz, 6H), 2.39-2.26 (m, 14H), 1.83 (s, 6H), 1.67-1.46 (m, 22H), 1.39-1.27 (m, 42H), 1.20 (d, J=6.4 Hz, 9H), 0.88 (t, J=6.4 Hz, 12H).

### Example 46—Compound 39

### O1-[2,2-bis[[8-(1-methylnonoxy)-8-oxo-octanoyl]oxymethyl]-3-[4-(2-pyrrolidin-1-ylethylcarbamoyloxy) decanoyloxy]propyl]O8-(1-methylnonyl) octanedioate

### Step 1:

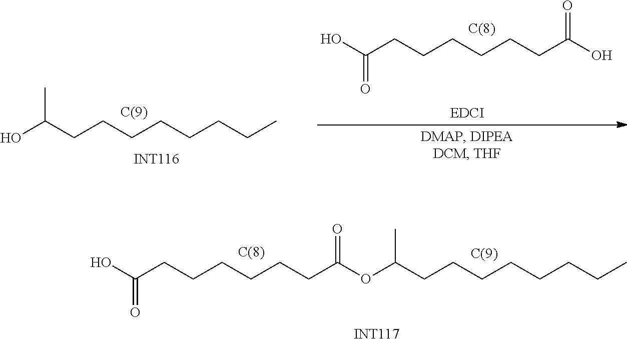

To a solution of decan-2-ol (20 g, 126.36 mmol, 1 eq) in DCM (1300 mL) was added EDCI (31.49 g, 164.27 mmol, 1.3 eq), octanedioic acid (110.05 g, 631.79 mmol, 5 eq), DMAP (1.54 g, 12.64 mmol, 0.1 eq) and DIPEA (40.83 g, 315.90 mmol, 55.02 mL, 2.5 eq) under N₂ atmosphere. The reaction mixture was stirred at 25° C. for 12 hr under N₂ atmosphere. The reaction mixture was diluted with H₂O (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound 8-(1-methylnonoxy)-8-oxo-octanoic acid (30 g, 95.40 mmol, 75.50% yield) was obtained as a colorless oil. ¹H NMR (400 MHz, CDCl₃) δ ppm 4.93-4.88 (m, 1H), 2.36 (t, J=7.6 Hz, 2H), 2.28 (t, J=7.6 Hz, 2H), 1.74-1.52 (m, 6H), 1.51-1.42 (m, 1H), 1.38-1.22 (m, 16H), 1.20 (d, J=6.2 Hz, 3H), 0.94-0.81 (m, 3H).

### Step 2:

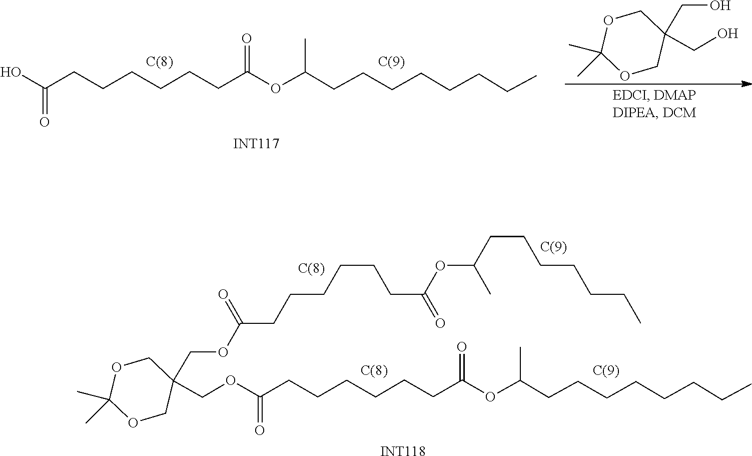

To a solution of 8-(1-methylnonoxy)-8-oxo-octanoic acid (3.97 g, 12.61 mmol, 1 eq) in DCM (50 mL) was added EDCI (2.90 g, 15.13 mmol, 1.2 eq), [5-(hydroxymethyl)-2,2-dimethyl-1,3-dioxan-5-yl]methanol (1 g, 5.68 mmol, 0.45 eq), DMAP (154.07 mg, 1.26 mmol, 0.1 eq) and DIPEA (4.07 g, 31.53 mmol, 5.49 mL, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[[2,2-dimethyl-5-[[8-(1-methylnonoxy)-8-oxo-octanoyl]oxymethyl]-1,3-dioxan-5-yl]methyl]O8-(1-methylnonyl) octanedioate (1.5 g, 1.95 mmol, 15.47% yield) was obtained as a colorless oil. ¹H NMR (400 MHz, CDCl₃) δ ppm 4.92-4.88 (m, 2H), 4.11 (s, 4H), 3.75 (s, 4H), 2.30 (m, 8H), 1.68-1.56 (m, 10H), 1.48 (m, 2H), 1.42 (s, 6H), 1.36-1.23 (m, 32H), 1.20 (d, J=6.2 Hz, 6H), 0.96-0.81 (m, 6H),

### Step 3:

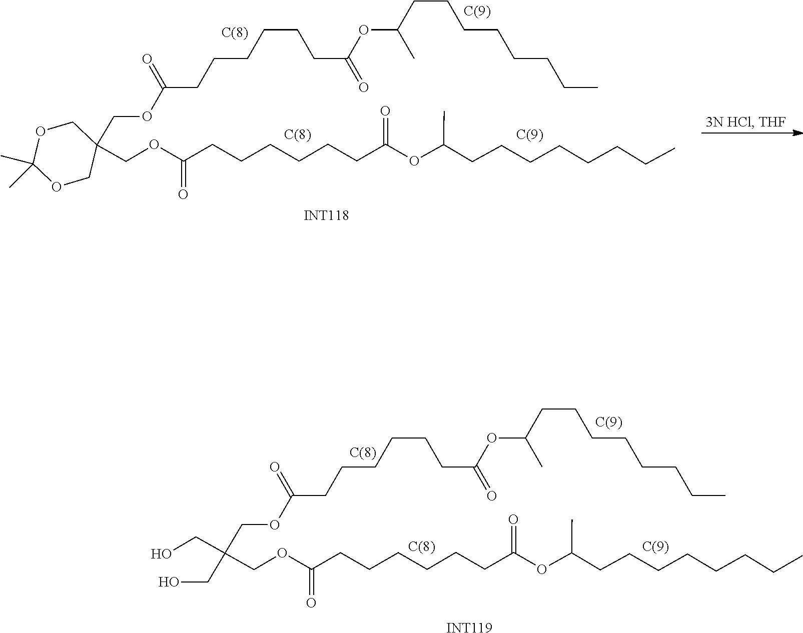

To a solution of O1-[[2,2-dimethyl-5-[[8-(1-methylnonoxy)-8-oxo-octanoyl]oxymethyl]-1,3-dioxan-5-yl]methyl]O8-(1-methylnonyl) octanedioate (1.5 g, 1.95 mmol, 1 eq) in THF (15 mL) was added HCl (3 M, 715.12 μL, 1.1 eq) at 0° C. under N₂ atmosphere. The reaction mixture was stirred at 25° C. for 2 hr under N₂ atmosphere. The reaction mixture was diluted with sat. NaHCO₃ (100 mL) and extracted with EtOAc 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis(hydroxymethyl)-3-[8-(1-methylnonoxy)-8-oxo-octanoyl]oxy-propyl]O8-(1-methylnonyl)octanedioate (0.8 g, 1.10 mmol, 56.26% yield) was obtained as a colorless oil. ¹H NMR (400 MHz, CDCl₃) δ ppm 4.92-4.88 (m, 2H), 4.15 (s, 4H), 3.58 (br s, 4H), 2.79 (br s, 2H), 2.35 (t, J=7.4 Hz, 4H), 2.27 (t, J=7.4 Hz, 4H), 1.66-1.53 (m, 10H), 1.49-1.41 (m, 2H), 1.39-1.23 (m, 32H), 1.20 (d, J=6.25 Hz, 6H), 0.97-0.82 (m, 6H).

### Step 4:

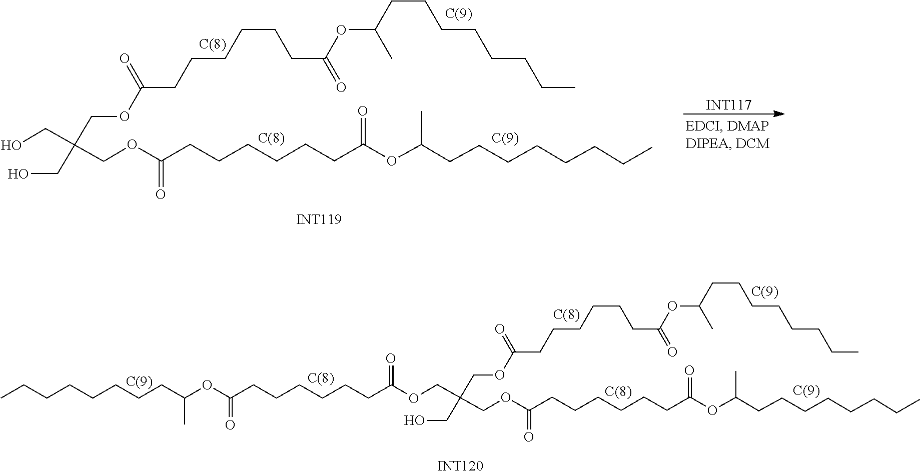

To a solution of O1-[2,2-bis(hydroxymethyl)-3-[8-(1-methylnonoxy)-8-oxo-octanoyl]oxy-propyl]O8-(1-methylnonyl) octanedioate (0.8 g, 1.10 mmol, 1 eq) in DCM (10 mL) was added EDCI (252.43 mg, 1.32 mmol, 1.2 eq), 8-(1-methylnonoxy)-8-oxo-octanoic acid (207.04 mg, 658.41 mol, 0.6 eq), DMAP (13.41 mg, 109.73 mol, 0.1 eq) and DIPEA (354.56 mg, 2.74 mmol, 477.84 μL, 2.5 eq) under N₂ atmosphere. The reaction mixture was stirred at 25° C. for 12 hr under N₂ atmosphere. The reaction mixture was diluted with H₂O (100 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2-(hydroxymethyl)-3-[8-(1-methylnonoxy)-8-oxo-octanoyl]oxy-2-[[8-(1-methylnonoxy)-8-oxo-octanoyl]oxymethyl]propyl]O8-(1methylnonyl) octanedioate (0.3 g, 292.55 mol, 26.66% yield) was obtained as a colorless oil. ¹H NMR (400 MHz, CDCl₃) δ ppm 4.92-4.87 (m, 3H), 4.12 (s, 6H), 3.51 (d, J=2.8 Hz, 2H), 2.62 (s, 1H), 2.33 (t, J=7.6 Hz, 6H), 2.27 (t, J=7.2 Hz, 6H), 1.64-1.61 (m, 10H), 1.49-1.42 (m, 6H), 1.36-1.32 (m, 14H), 1.27 (s, 34H), 1.20 (d, J=6.4 Hz, 9H), 0.88 (t, J=6.4 Hz, 9H).

### Step 5:

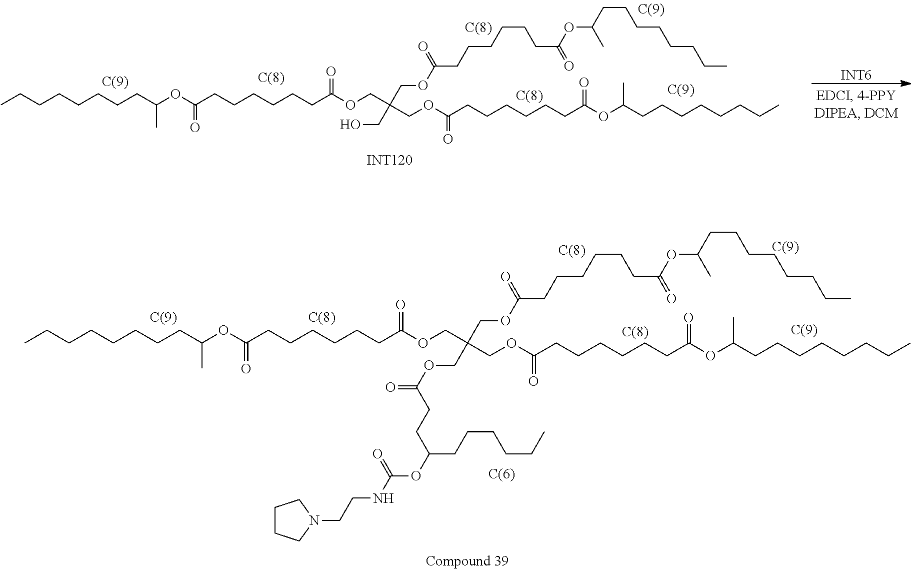

To a solution of O1-[2-(hydroxymethyl)-3-[8-(1-methylnonoxy)-8-oxo-octanoyl]oxy-2-[[8-(1-methylnonoxy)-8-oxo-octanoyl]oxymethyl]propyl]O8-(1-methylnonyl) octanedioate (1 g, 975.16 mol, 1 eq) in DCM (13 mL) was added EDCI (224.33 mg, 1.17 mmol, 1.2 eq), 4-(2-pyrrolidin-1-ylethylcarbamoyloxy)decanoic acid (288.26 mg, 877.64 mol, 0.9 eq), 4-pyrrolidin-1-ylpyridine (14.45 mg, 97.52 mol, 0.1 eq) and DIPEA (315.08 mg, 2.44 mmol, 424.64 μL, 2.5 eq) under N₂ atmosphere. The reaction mixture was stirred at 25° C. for 12 hr under N₂ atmosphere. The reaction mixture was diluted with H₂O (50 mL) and extracted with DCM 100 mL (25 mL*4). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2,2-bis[[8-(1-methylnonoxy)-8-oxo-octanoyl]oxymethyl]-3-[4-(2-pyrrolidin-1-ylethylcarbamoyloxy) decanoyloxy]propyl]08-(1-methylnonyl) octanedioate (0.1 g, 74.86 mol, 7.68% yield) was obtained as a colorless oil. ¹H NMR (400 MHz, CDCl₃) δ ppm 5.31-5.29 (s, 1H), 4.93-4.86 (m, 3H), 4.75 (s, 1H), 4.11 (s, 8H), 3.33 (d, J=4 Hz, 2H), 2.61 (d, J=25.6 Hz, 6H), 2.40-2.25 (m, 14H), 1.90-1.81 (m, 6H), 1.64-1.55 (m, 18H), 1.49-1.42 (m, 4H), 1.35-1.32 (m, 14H), 1.27 (s, 40H), 1.20 (d, J=6.4 Hz, 9H), 0.88 (t, J=6.4 Hz, 12H).

### Example 47—Compound 40

### O1-[2,2-bis[[7-(1-methylnonoxy)-7-oxo-heptanoyl]oxymethyl]-3-[4-(2-pyrrolidin-1-ylethylcarbamoyloxy)decanoyloxy]propyl]O7-(1-methylnonyl)heptanedioate

### Step 1:

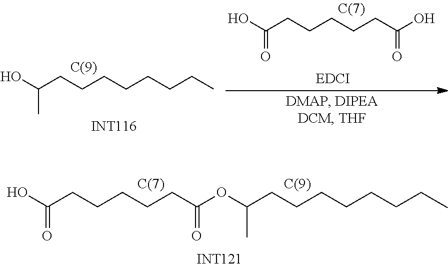

To a solution of decan-2-ol (20 g, 126.36 mmol, 1 eq) in DCM (1210 mL) was added EDCI (29.07 g, 151.63 mmol, 1.2 eq), heptanedioic acid (101.19 g, 631.79 mmol, 5 eq), DMAP (1.54 g, 12.64 mmol, 0.1 eq) and DIPEA (40.83 g, 315.90 mmol, 55.02 mL, 2.5 eq) under N₂ atmosphere. The reaction mixture was stirred at 25° C. for 12 hr under N₂ atmosphere. The reaction mixture was diluted with H₂O (500 mL) and extracted with DCM 1500 mL (500 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound 7-(1-methylnonoxy)-7-oxo-heptanoic acid (30 g, 99.86 mmol, 79.03% yield) was obtained as a colorless oil. ¹H NMR (400 MHz, CDCl₃) δ ppm 4.92-4.88 (m, 1H), 2.36 (t, J=7.47 Hz, 2H), 2.29 (t, J=7.47 Hz, 2H), 1.74-1.60 (m, 4H), 1.59-1.36 (m, 4H), 1.27 (br s, 12H), 1.20 (d, J=6.27 Hz, 3H), 0.93-0.82 (m, 3H).

### Step 2:

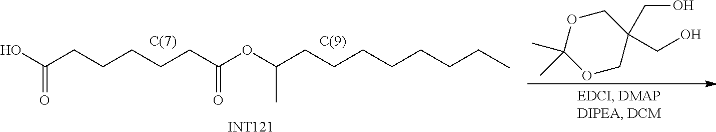

To a solution of 7-(1-methylnonoxy)-7-oxo-heptanoic acid (3.79 g, 12.61 mmol, 1 eq) in DCM (50 mL) was added EDCI (2.90 g, 15.13 mmol, 1.2 eq), [5-(hydroxymethyl)-2,2-dimethyl-1,3-dioxan-5-yl]methanol (1 g, 5.68 mmol, 0.45 eq), DMAP (154.07 mg, 1.26 mmol, 0.1 eq) and DIPEA (4.07 g, 31.53 mmol, 5.49 mL, 2.5 eq) under N₂ atmosphere. The reaction mixture was stirred at 25° C. for 12 hr under N₂ atmosphere. The reaction mixture was diluted with H₂O (200 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[[2,2-dimethyl-5-[[7-(1-methylnonoxy)-7-oxo-heptanoyl]oxymethyl]-1,3-dioxan-5-yl]methyl]O7-(1-methylnonyl)heptanedioate (1.5 g, 2.02 mmol, 16.05% yield) was obtained as a colorless oil. ¹H NMR (400 MHz, CDCl₃) δ ppm 4.92-4.88 (m, 2H), 4.11 (s, 4H), 3.74 (s, 4H), 2.42-2.21 (m, 8H), 1.71-1.57 (m, 10H), 1.47 (br d, J=8.0 Hz, 2H), 1.42 (s, 6H), 1.38-1.21 (m, 28H), 1.20 (d, J=6.2 Hz, 6H), 0.97-0.77 (m, 6H).

### Step 3:

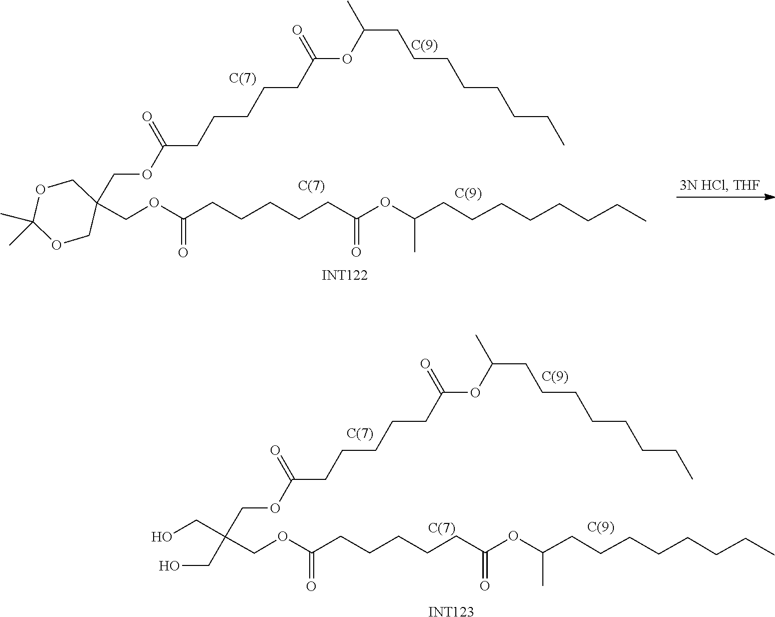 [SMILES: CCCCCCCCC(C)OC(=O)CCCCCC(=O)OCC1(COC(=O)CCCCCC(=O)OC(C)CCCCCCCC)COC(C)(C)OC1 ; "INT122" ; CCCCCCCCC(C)OC(=O)CCCCCC(=O)OCC(CO)(CO)COC(=O)CCCCCC(=O)OC(C)CCCCCCCC ; "INT123"]

To a solution of O1-[[2,2-dimethyl-5-[[7-(1-methylnonoxy)-7-oxo-heptanoyl]oxymethyl]-1,3-dioxan-5-yl]methyl]O7-(1-methylnonyl) heptanedioate (1.5 g, 2.02 mmol, 1 eq) in THF (15 mL) was added HCl (3 M, 742.20 μL, 1.1 eq) at 0° C. under N₂ atmosphere. The reaction mixture was stirred at 25° C. for 2 hr under N₂ atmosphere. The reaction mixture was diluted with sat. NaHCO₃ (100 mL) and extracted with EtOAc 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis(hydroxymethyl)-3-[7-(1-methylnonoxy)-7-oxo-heptanoyl]oxy-propyl]O7-(1 -methylnonyl) heptanedioate (0.8 g, 1.14 mmol, 56.38% yield) was obtained as a colorless oil. ¹H NMR (400 MHz, CDCl₃) δ ppm 4.92-4.87 (m, 2H), 4.15 (s, 4H), 3.59 (br d, J=4.50 Hz, 4H), 2.80 (br s, 2H), 2.37 (t, J=7.44 Hz, 4H), 2.29 (t, J=7.38 Hz, 4H), 1.69-1.60 (m, 10H), 1.48-1.34 (m, 6H), 1.27 (br s, 24H), 1.20 (d, J=6.25 Hz, 6H), 0.89 (t, J=6.75 Hz, 6H).

### Step 4:

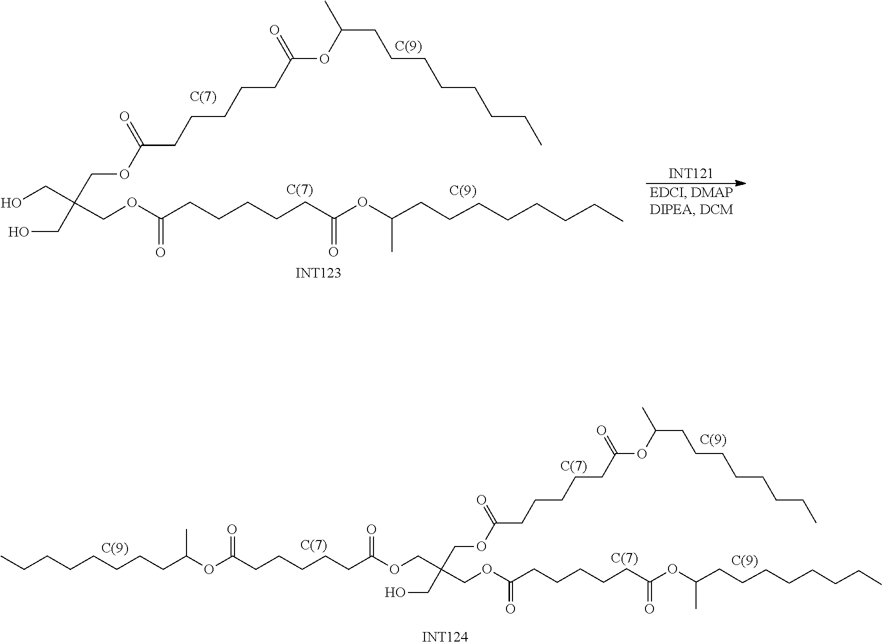

To a solution of O1-[2,2-bis(hydroxymethyl)-3-[7-(1-methylnonoxy)-7-oxo-heptanoyl]oxy-propyl]O7-(1-methylnonyl) heptanedioate (0.8 g, 1.14 mmol, 1 eq) in DCM (10 mL) was added EDCI (262.54 mg, 1.37 mmol, 1.2 eq), 7-(1-methylnonoxy)-7-oxo-heptanoic acid (205.72 mg, 684.76 mol, 0.6 eq), DMAP (13.94 mg, 114.13 mol, 0.1 eq) and DIPEA (368.75 mg, 2.85 mmol, 496.97 L, 2.5 eq) under N₂ atmosphere. The reaction mixture was stirred at 25° C. for 12 hr under N₂ atmosphere. The reaction mixture was diluted with H₂O (100 mL) and extracted with DCM 400 mL (100 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O1-[2-(hydroxymethyl)-3-[7-(1-methylnonoxy)-7-oxo-heptanoyl]oxy-2-[[7-(1-methylnonoxy)-7-oxo-heptanoyl]oxymethyl]propyl]O7-(1-methylnonyl) heptanedioate (0.3 g, 305.06 mol, 26.73% yield) was obtained as a colorless oil. ¹H NMR (400 MHz, CDCl₃) δ ppm 4.93-4.86 (m, 3H), 4.11 (s, 6H), 3.52 (d, J=4.8 Hz, 2H), 2.68 (s, 1H), 2.34 (t, J=7.6 Hz, 6H), 2.90 (t, J=7.6 Hz, 6H), 1.68-1.60 (m, 14H), 1.49-1.42 (m, 4H), 1.40-1.27 (m, 42H), 1.20 (d, J=6.0 Hz, 9H), 0.88 (t, J=13.6 Hz, 9H).

### Step 5:

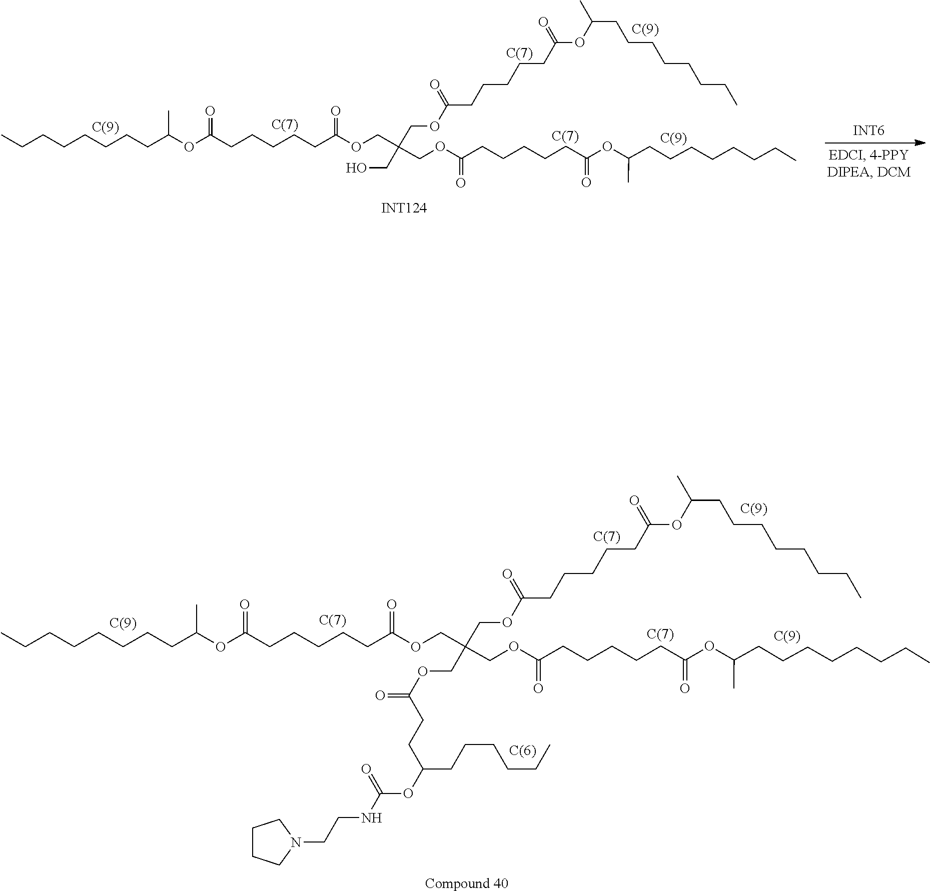

To a solution of O1-[2-(hydroxymethyl)-3-[7-(1-methylnonoxy)-7-oxo-heptanoyl]oxy-2-[[7-(1-methylnonoxy)-7-oxo-heptanoyl]oxymethyl]propyl]O7-(1-methylnonyl) heptanedioate (1 g, 1.02 mmol, 1 eq) in DCM (13 mL) was added EDCI (233.93 mg, 1.22 mmol, 1.2 eq), 4-(2-pyrrolidin-1-ylethylcarbamoyloxy) decanoic acid (300.59 mg, 915.19 mol, 0.9 eq), 4-pyrrolidin-1-ylpyridine (15.07 mg, 101.69 mol, 0.1 eq) and DIPEA (328.56 mg, 2.54 mmol, 442.81 L, 2.5 eq) under N₂ atmosphere. The reaction mixture was stirred at 25° C. for 12 hr under N₂ atmosphere. The reaction mixture was diluted with H₂O (50 mL) and extracted with DCM 50 mL (25 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O1-[2,2-bis[[7-(1-methylnonoxy)-7-oxo-heptanoyl]oxymethyl]-3-[4-(2-pyrrolidin-1-ylethylcarbamoyloxy)decanoyloxy]propyl]O7-(1-methylnonyl)heptanedioate (0.1 g, 77.29 mol, 7.60% yield) was obtained as a colorless oil. ¹H NMR (400 MHz, CDCl₃) δ ppm 5.39-5.28 (s, 1H), 4.92-4.87 (m, 3H), 4.75-4.74 (s, 1H), 4.11 (s, 8H), 3.34 (d, J=3.6 Hz, 2H), 2.63 (d, J=1.6 Hz, 6H), 2.39-2.26 (m, 14H), 1.91-1.77 (m, 6H), 1.67-1.44 (m, 26H), 1.38-1.27 (m, 44H), 1.20 (d, J=6.4 Hz, 9H), 0.88 (t, J=13.6 Hz, 12H).

### Example 48—Compound 41

### O1-[2,2-bis[[8-(1-methyldecoxy)-8-oxo-octanoyl]oxymethyl]-3-[4-(2-pyrrolidin-1-ylethylcarbamoyloxy)decanoyloxy]propyl]O8-(1-methyldecyl) octanedioate

### Step 1:

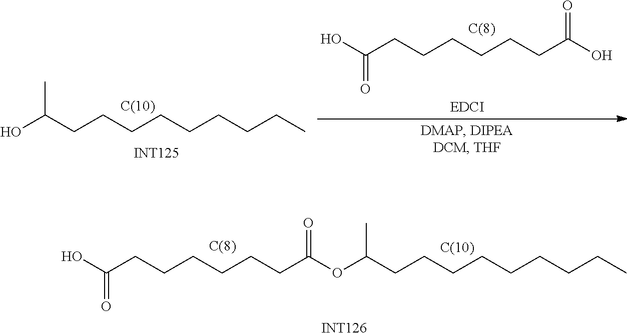

To a solution of undecan-2-ol (15 g, 87.05 mmol, 1 eq) in DCM (450 mL) and THF (450 mL) was added EDCI (20.03 g, 104.46 mmol, 1.2 eq), octanedioic acid (75.82 g, 435.27 mmol, 5 eq), DIPEA (28.13 g, 217.64 mmol, 37.91 mL, 2.5 eq) and DMAP (1.06 g, 8.71 mmol, 0.1 eq) under N₂ atmosphere. The reaction mixture was stirred at 25° C. for 12 hr under N₂ atmosphere. The reaction mixture was diluted with H₂O (1000 mL) and extracted with DCM 2100 mL (700 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 8-(1-methyldecoxy)-8-oxo-octanoic acid (15 g, 45.66 mmol, 52.45% yield) was obtained as a colorless oil.

### Step 2:

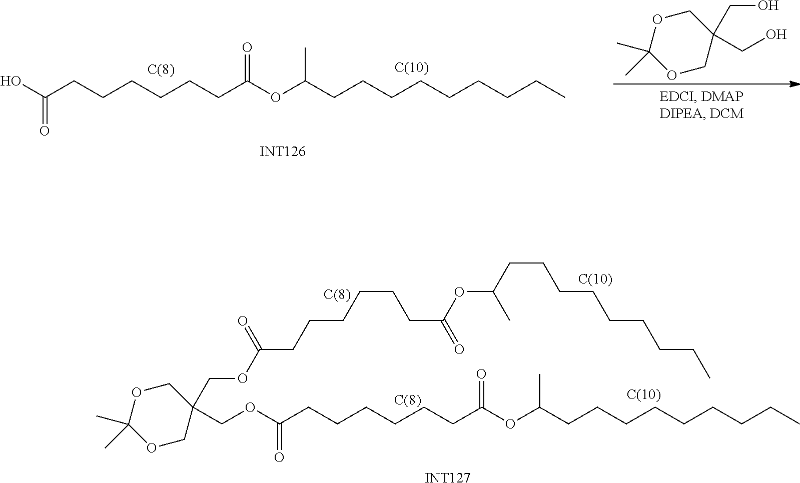

To a solution of 8-(1-methyldecoxy)-8-oxo-octanoic acid (4.14 g, 12.61 mmol, 1 eq) in DCM (50 mL) was added EDCI (2.90 g, 15.13 mmol, 1.2 eq), DIPEA (4.07 g, 31.53 mmol, 5.49 mL, 2.5 eq), [5-(hydroxymethyl)-2,2-dimethyl-1,3-dioxan-5-yl]methanol (1 g, 5.68 mmol, 0.45 eq) and DMAP (154.07 mg, 1.26 mmol, 0.1 eq) under N₂ atmosphere. The reaction mixture was stirred at 25° C. for 12 hr under N₂ atmosphere. The reaction mixture was diluted with H₂O (100 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[[2,2-dimethyl-5-[[8-(1-methyldecoxy)-8-oxo-octanoyl]oxymethyl]-1,3-dioxan-5-yl]methyl]O8-(1-methyldecyl) octanedioate (1.5 g, 1.88 mmol, 14.92% yield) was obtained as a colorless oil.

### Step 3:

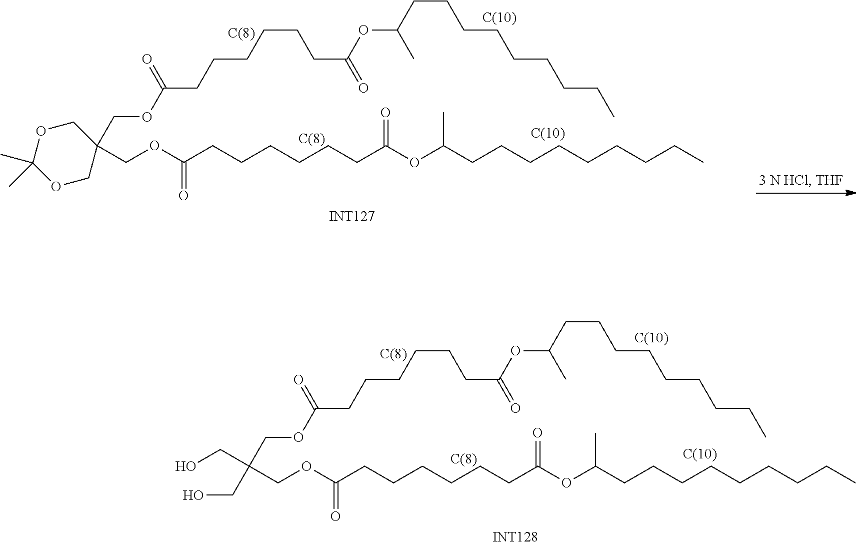 [SMILES: CCCCCCCCCC(C)OC(=O)CCCCCCC(=O)OCC1(COC(=O)CCCCCCC(=O)OC(C)CCCCCCCCC)COC(C)(C)OC1 ; "INT127" ; CCCCCCCCCC(C)OC(=O)CCCCCCC(=O)OCC(CO)(CO)COC(=O)CCCCCCC(=O)OC(C)CCCCCCCCC ; "INT128"]

To a solution of O1-[[2,2-dimethyl-5-[[8-(1-methyldecoxy)-8-oxo-octanoyl]oxymethyl]-1,3-dioxan-5-yl]methyl]O8-(1-methyldecyl) octanedioate (1.5 g, 1.88 mmol, 1 eq) in THF (15 mL) was added HCl (3 M, 689.96 L, 1.1 eq) at 0° C. under N₂ atmosphere. The reaction mixture was stirred at 25° C. for 12 hr under N₂ atmosphere. The reaction mixture was diluted with sat. NaHCO₃ (100 mL) and extracted with EtOAc 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2,2-bis(hydroxymethyl)-3-[8-(1-methyldecoxy)-8-oxo-octanoyl]oxy-propyl]O8-(1-methyldecyl) octanedioate (0.8 g, 1.06 mmol, 56.16% yield) was obtained as a colorless oil.

### Step 4:

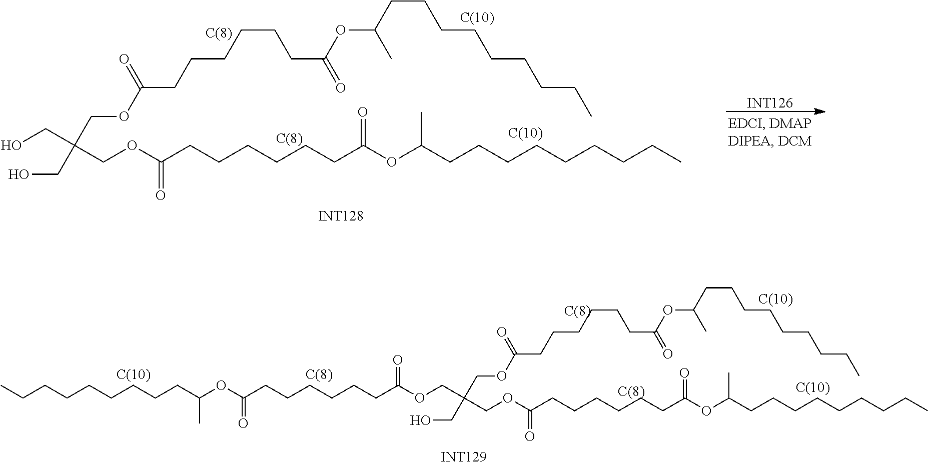

To a solution of O1-[2,2-bis(hydroxymethyl)-3-[8-(1-methyldecoxy)-8-oxo-octanoyl]oxy-propyl]O8-(1-methyldecyl) octanedioate (0.8 g, 1.06 mmol, 1 eq) in DCM (10 mL) was added EDCI (243.08 mg, 1.27 mmol, 1.2 eq), 8-(1-methyldecoxy)-8-oxo-octanoic acid (208.26 mg, 634.01 (mol, 0.6 eq), DIPEA (341.42 mg, 2.64 mmol, 460.14 μL, 2.5 eq) and DMAP (12.91 mg, 105.67 μmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (10 mL) and extracted with DCM 30 mL (10 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/i to 0/1). Compound O1-[2-(hydroxymethyl)-3-[8-(1-methyldecoxy)-8-oxo-octanoyl]oxy-2-[[8-(1-methyldecoxy)-8-oxo-octanoyl]oxymethyl]propyl]O8-(1-methyldecyl) octanedioate (0.35 g, 327.85 μmol, 31.03% yield) was obtained as a colorless oil. ¹H NMR (400 MHz, CDCl₃) δ ppm 4.91-4.87 (m, 3H), 4.11 (s, 6H), 3.50 (s, 2H), 2.33 (t, J=14.8 Hz, 6H), 2.27 (t, J=4.0 Hz, 6H), 2.60 (t, J=7.6 Hz, 6H), 1.92-1.78 (m, 2H), 1.63-1.54 (m, 14H), 1.49-1.44 (m, 4H), 1.35-1.26 (m, 53H), 1.20 (d, J=6.0 Hz, 9H).

### Step 5:

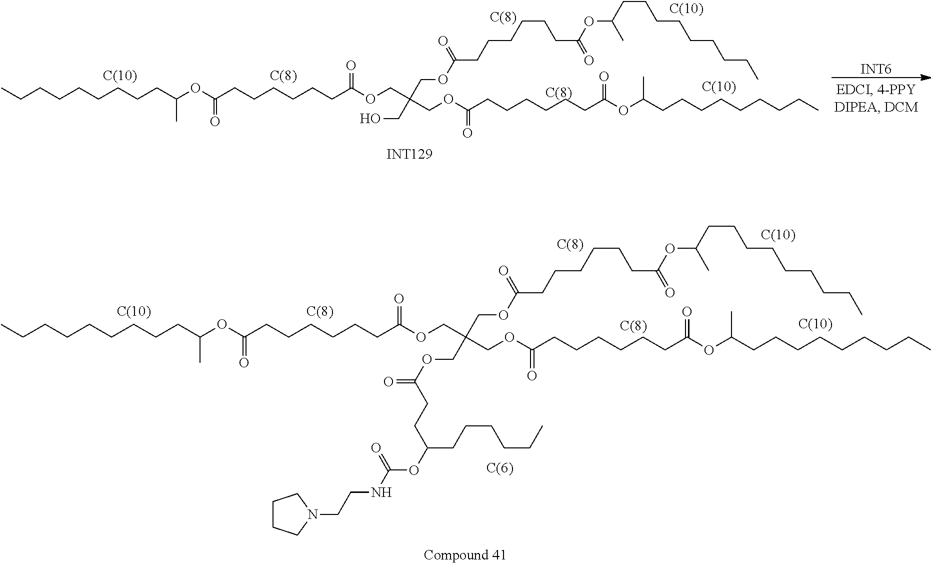

To a solution of O1-[2-(hydroxymethyl)-3-[8-(1-methyldecoxy)-8-oxo-octanoyl]oxy-2-[[8-(1 methyldecoxy-8-oxo-octanoyl]oxymethyl]propyl]O8-(1-methyldecyl) octanedioate (0.35 g, 327.85 mol, 1 eq) in DCM (15 mL) was added EDCI (75.42 mg, 393.42 mol, 1.2 eq), 4-(2-pyrrolidin-1-ylethylcarbamoyloxy)decanoic acid (129.22 mg, 393.42 mol, 1.2 eq), DIPEA (105.93 mg, 819.63 mol, 142.76 L, 2.5 eq) and 4-pyrrolidin-1-ylpyridine (4.86 mg, 32.79 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (10 mL) and extracted with DCM 30 mL (10 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2,2-bis[[8-(1-methyldecoxy)-8-oxo-octanoyl]oxymethyl]-3-[4-(2-pyrrolidin-1-ylethylcarbamoyloxy)decanoyloxy]propyl]O8-(1-methyldecyl) octanedioate (0.12 g, 87.08 mol, 26.56% yield) was obtained as a colorless oil. ¹H NMR (400 MHz, CDCl₃) δ ppm 5.35-5.30 (m, 1H), 4.93-4.85 (m, 3H), 4.75 (s, 1H), 4.14-4.11 (m, 8H), 3.32 (t, J=1.2 Hz, 2H), 2.60 (d, J=24.8 Hz, 6H), 2.42-2.25 (m, 14H), 1.90-1.85 (m, 6H), 1.80 (s, 4H), 1.63-1.54 (m, 16H), 1.49-1.42 (m, 4H), 1.35-1.26 (m, 62H), 1.20 (d, J=6.4 Hz, 9H), 0.88 (t, J=6.4 Hz, 12H).

### Example 49—Compound 42

### O1-[2,2-bis[[7-(1-methyldecoxy)-7-oxo-heptanoyl]oxymethyl]-3-[4-(2-pyrrolidin-1-ylethylcarbamoyloxy)decanoyloxy]propyl]O7-(1-methyldecyl) heptanedioate

### Step 1:

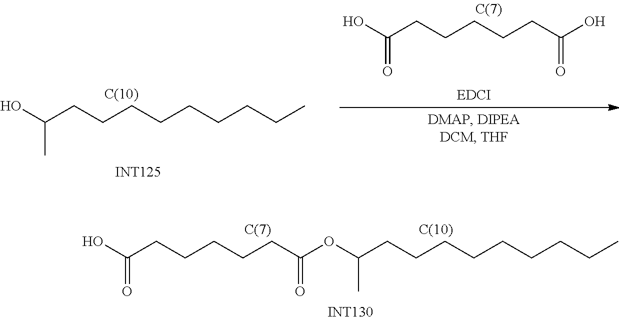

To a solution of undecan-2-ol (15 g, 87.05 mmol, 1 eq) in DCM (400 mL) and THF (400 mL) was added EDCI (20.03 g, 104.46 mmol, 1.2 eq), heptanedioic acid (69.72 g, 435.27 mmol, 5 eq), DIPEA (28.13 g, 217.64 mmol, 37.91 mL, 2.5 eq) and DMAP (1.06 g, 8.71 mmol, 0.1 eq) under N₂ atmosphere. The reaction mixture was stirred at 25° C. for 20 hr under N₂ atmosphere. The reaction mixture was concentrated under reduced pressure to remove solvent. The residue was diluted with H₂O (1000 mL) and extracted with DCM 3000 mL (1000 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 7-(1-methyldecoxy)-7-oxo-heptanoic acid (15 g, 47.70 mmol, 54.79% yield) was obtained as a colorless oil.

### Step 2:

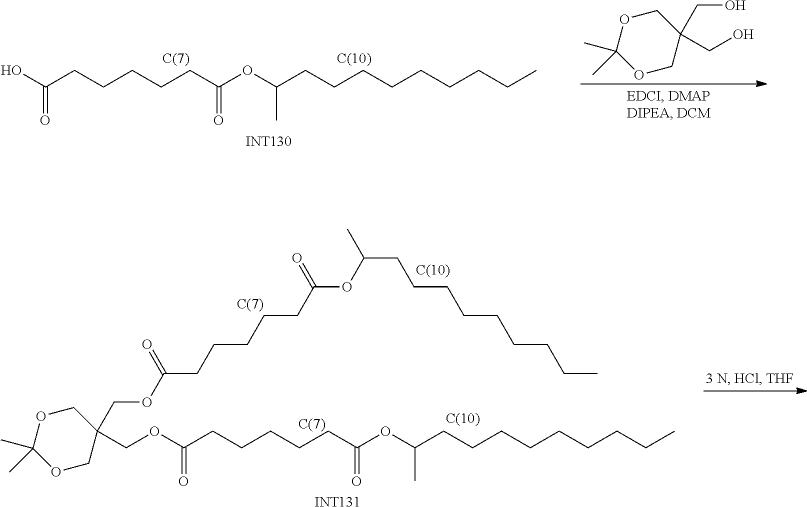

To a solution of 7-(1-methyldecoxy)-7-oxo-heptanoic acid (3.97 g, 12.61 mmol, 1 eq) in DCM (50 mL) was added EDCI (2.90 g, 15.13 mmol, 1.2 eq), [5-(hydroxymethyl)-2,2-dimethyl-1,3-dioxan-5-yl]methanol (1 g, 5.68 mmol, 0.45 eq), DIPEA (4.07 g, 31.53 mmol, 5.49 mL, 2.5 eq) and DMAP (154.07 mg, 1.26 mmol, 0.1 eq) under N₂ atmosphere. The reaction mixture was stirred at 20° C. for 12 hr under N₂ atmosphere. The reaction mixture was concentrated under reduced pressure to remove solvent. The residue was diluted with H₂O (100 mL) and extracted with DCM 300 mL (100 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[[2,2-dimethyl-5-[[7-(1-methyldecoxy)-7-oxo-heptanoyl]oxymethyl]-1,3-dioxan-5-yl]methyl]O7-(1-methyldecyl) heptanedioate (1.5 g, 1.95 mmol, 15.47% yield) was obtained as a colorless oil. ¹H NMR (400 MHz, CDCl₃) δ ppm 4.92-4.87 (m, 2H), 4.11 (s, 4H), 3.74 (s, 4H), 2.33 (t, J=7.2 Hz, 4H), 2.28 (t, J=7.6 Hz, 4H), 1.68-1.55 (m, 12H), 1.42 (s, 6H), 1.38-1.32 (m, 6H), 1.27 (s, 26H), 1.20 (d, J=6 Hz, 6H), 0.99 (t, J=6.8 Hz, 6H).

### Step 3:

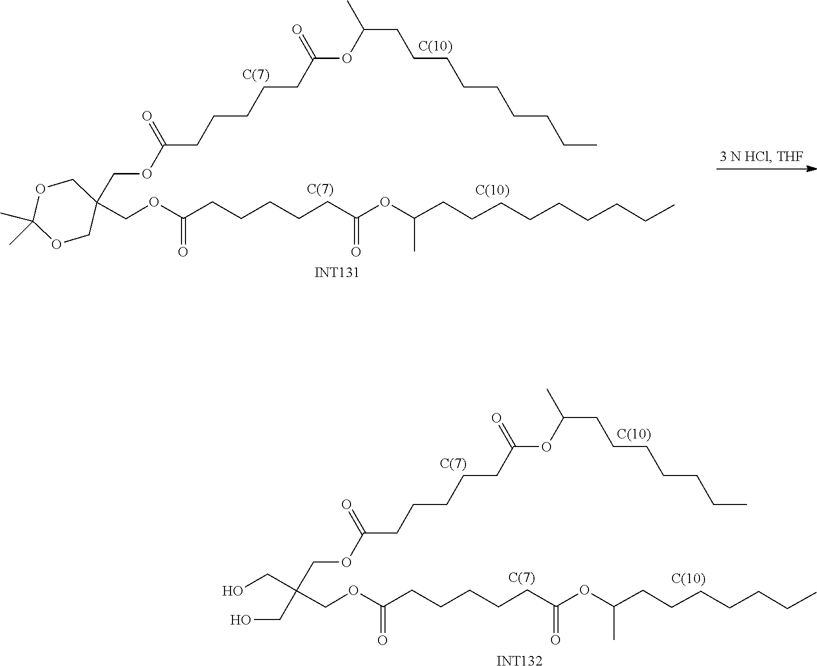 [SMILES: CCCCCCCCCC(C)OC(=O)CCCCCC(=O)OCC1(COC(=O)CCCCCC(=O)OC(C)CCCCCCCCC)COC(C)(C)OC1 ; "INT131" ; CCCCCCCC(C)OC(=O)CCCCCC(=O)OCC(CO)(CO)COC(=O)CCCCCC(=O)OC(C)CCCCCCC ; "INT132"]

To a solution of O1-[[2,2-dimethyl-5-[[7-(1-methyldecoxy)-7-oxo-heptanoyl]oxymethyl]-1,3-dioxan-5-yl]methyl]O7-(1-methyldecyl) heptanedioate (1.5 g, 1.95 mmol, 1 eq) in THF (15 mL) was added HCl (3 M, 715.12 L, 1.1 eq) at 0° C. under N₂ atmosphere. The reaction mixture was stirred at 25° C. for 2 hr under N₂ atmosphere. The reaction mixture was diluted with sat. NaHCO₃ (100 mL) and extracted with EtOAc 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2,2-bis(hydroxymethyl)-3-[7-(1-methyldecoxy)-7-oxo-heptanoyl]oxy-propyl]O7-(1-methyldecyl) heptanedioate (0.8 g, 1.10 mmol, 56.26% yield) was obtained as a colorless oil. ¹H NMR (400 MHz, CDCl₃) δ ppm 4.90-4.86 (m, 2H), 4.13 (s, 4H), 3.58 (s, 4H), 2.36 (t, J=7.6 Hz, 4H), 2.28 (t, J=7.2 Hz, 4H), 1.67-1.59 (m, 9H), 1.57-1.45 (m, 4H), 1.38-1.26 (m, 33H), 1.19 (d, J=6.0 Hz, 6H), 0.88 (t, J=6.4 Hz, 6H).

### Step 4:

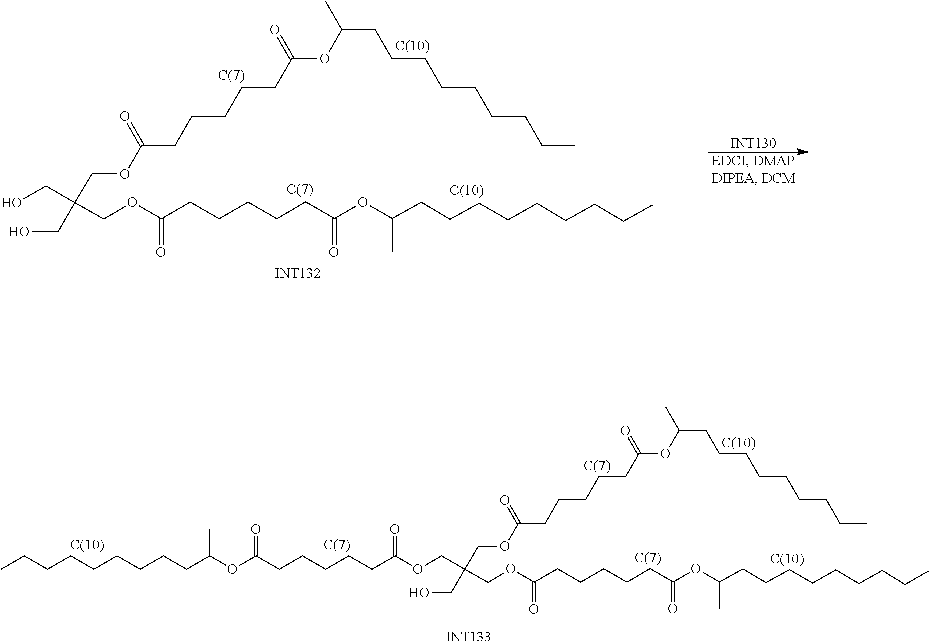

To a solution of O1-[2,2-bis(hydroxymethyl)-3-[7-(1-methyldecoxy)-7-oxo-heptanoyl]oxy-propyl]O7-(1-methyldecyl) heptanedioate (0.8 g, 1.10 mmol, 1 eq) in DCM (10 mL) was added EDCI (252.43 mg, 1.32 mmol, 1.2 eq), 7-(1-methyldecoxy)-7-oxo-heptanoic acid (207.04 mg, 658.41 mol, 0.6 eq), DIPEA (354.56 mg, 2.74 mmol, 477.84 μL, 2.5 eq) and DMAP (13.41 mg, 109.73 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2-(hydroxymethyl)-3-[7-(1-methyldecoxy)-7-oxo-heptanoyl]oxy-2-[[7-(1-methyldecoxy)-7-oxo-heptanoyl]oxymethyl]propyl]O7-(1-methyldecyl) heptanedioate (0.45 g, 438.82 mol, 39.99% yield) was obtained as a colorless oil. ¹H NMR (400 MHz, CDCl₃) δ ppm 4.91-4.87 (m, 3H), 4.11 (s, 6H), 3.51 (d, J=6.4 Hz, 2 H), 2.68 (t, J=6.4 Hz, 1H), 2.34 (t, J=7.2 Hz, 6H), 2.28 (t, J=7.6 Hz, 6H), 1.67-1.56 (m, 18H), 1.49-1.44 (m, 3H), 1.38-1.27 (m, 48H), 1.20 (d, J=6.0 Hz, 9H), 0.88 (t, J=6.4 Hz, 9H).

### Step 5:

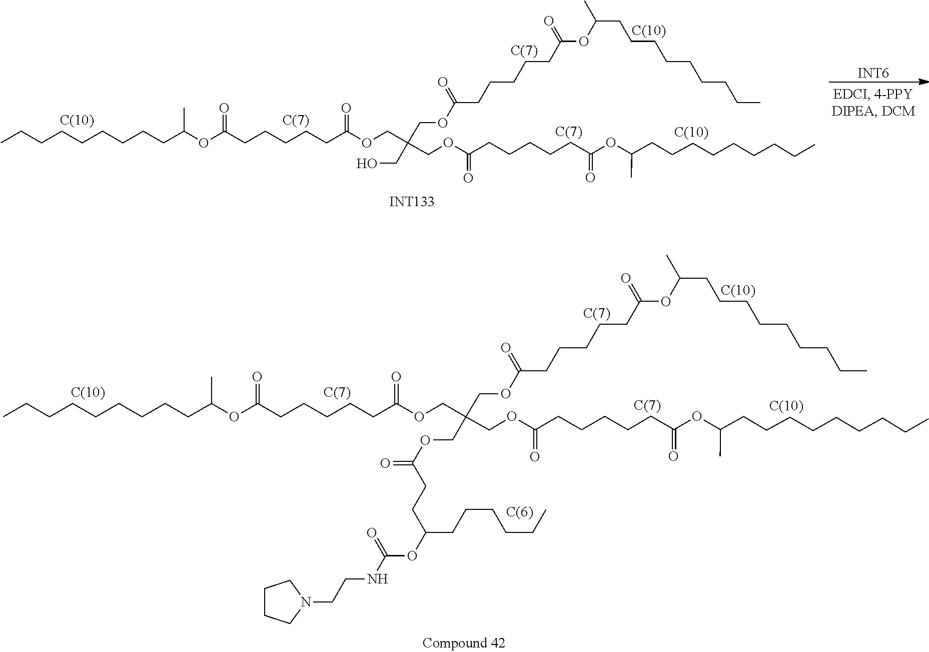

To a solution of O1-[2-(hydroxymethyl)-3-[7-(1-methyldecoxy)-7-oxo-heptanoyl]oxy-2-[[7-(1-methyldecoxy)-7-oxo-heptanoyl]oxymethyl]propyl]O7-(1-methyldecyl) heptanedioate (0.25 g, 243.79 mol, 1 eq) in DCM (10 mL) was added EDCI (56.08 mg, 292.55 mol, 1.2 eq), 4-pyrrolidin-1-ylpyridine (3.61 mg, 24.38 mol, 0.1 eq), 4-(2-pyrrolidin-1-ylethylcarbamoyloxy)decanoic acid (96.09 mg, 292.55 mol, 1.2 eq) and DIPEA (78.77 mg, 609.47 mol, 106.16 μL, 2.5 eq) under N₂ atmosphere. The reaction mixture was stirred at 25° C. for 12 hr under N₂ atmosphere. The reaction mixture was concentrated under reduced pressure to remove solvent. The residue was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O1-[2,2-bis[[7-(1-methyldecoxy)-7-oxo-heptanoyl]oxymethyl]-3-[4-(2-pyrrolidin-1-ylethylcarbamoyloxy)decanoyloxy]propyl]O7-(1-methyldecyl) heptanedioate was obtained as a colorless oil. ¹H NMR (400 MHz, CDCl₃) δ ppm 5.37-5.32 (m, 1H), 4.93-4.85 (m, 3H), 4.75 (s, 1H), 4.11 (s, 8H), 3.34 (s, 2H), 2.67- 2.56 (m, 4H), 2.38-2.26 (m, 14H), 1.89-1.75 (m, 6H), 1.67-1.57 (m, 20H), 1.49-1.44 (m, 4H), 1.38-1.27 (m, 54H), 1.23 (d, J=6.4 Hz, 9H), 0.88 (t, J=6.8 Hz, 12H).

### Example 50—Compound 43

### O8-[2,2-bis[[8-[(Z)-oct-3-enoxy]-8-oxo-octanoyl]oxymethyl]-3-[4-(2-pyrrolidin-1-ylethylcarbamoyloxy)decanoyloxy]propyl]O1-[(Z)-oct-3-enyl]octanedioate

### Step 1:

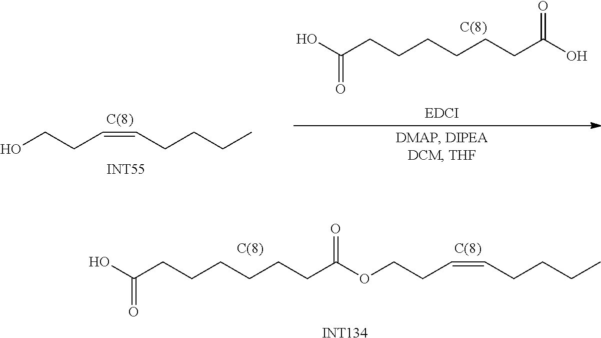

To a mixture of (Z)-oct-3-en-1-ol (20 g, 155.99 mmol, 1 eq), DMAP (1.91 g, 15.60 mmol, 0.1 eq), DIPEA (50.40 g, 389.98 mmol, 67.93 mL, 2.5 eq) and EDCI (35.88 g, 187.19 mmol, 1.2 eq) in DCM (800 mL) and THF (800 mL) was added octanedioic acid (135.86 g, 779.96 mmol, 5 eq) under N₂ atmosphere. The reaction mixture was stirred at 25° C. for 12 hr under N₂ atmosphere. The reaction mixture was diluted with H₂O (2000 mL) and extracted with ethyl acetate 6000 mL (2000 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 8-[(Z)-oct-3-enoxy]-8-oxo-octanoic acid (30 g, 105.49 mmol, 67.62% yield) was obtained as colorless oil. ¹H NMR (400 MHz, CDCl₃) δ ppm 11.41 (br s, 1H), 5.56-5.45 (m, 1H), 5.41-5.28 (m, 1H), 4.07 (t, J=6.8 Hz, 2H), 2.43-2.26 (m, 6H), 2.05 (q, J=6.6 Hz, 2H), 1.72-1.55 (m, 4H), 1.34-1.32 (m, 8H), 1.04-0.87 (m, 3H).

### Step 2:

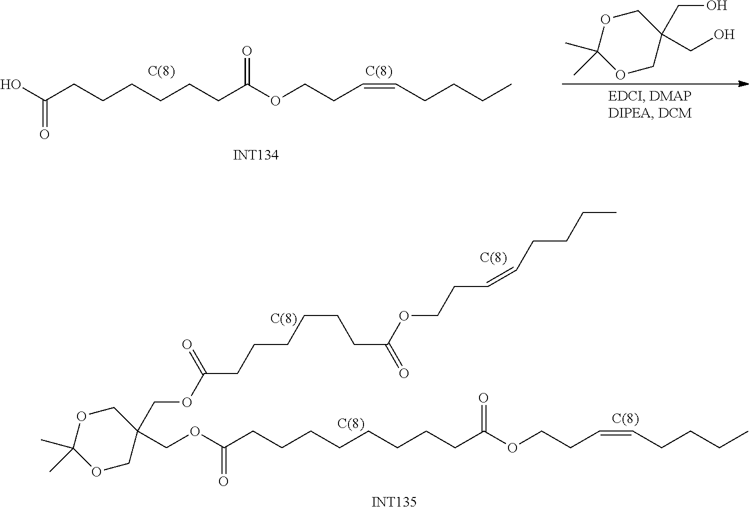

To a mixture of 8-[(Z)-oct-3-enoxy]-8-oxo-octanoic acid (10.76 g, 37.83 mmol, 1 eq), DIPEA (12.22 g, 94.58 mmol, 16.47 mL, 2.5 eq), DMAP (462.20 mg, 3.78 mmol, 0.1 eq) and EDCI (8.70 g, 45.40 mmol, 1.2 eq) in DCM (150 mL) was added [5-(hydroxymethyl)-2,2-dimethyl-1,3-dioxan-5-yl]methanol (3 g, 17.03 mmol, 0.45 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[[2,2-dimethyl-5-[[8-[(Z)-oct-3-enoxy]-8-oxo-octanoyl]oxymethyl]-1,3-dioxan-5-yl]methyl]O1-[(Z)-oct-3-enyl]octanedioate (3.5 g, 4.94 mmol, 13.05% yield) was obtained as a colorless oil. ¹H NMR (400 MHz, CDCl₃) δ ppm 5.52-5.49 (m, 2H), 5.35-5.33 (m, 2H), 4.11 (s, 4H), 4.07 (t, J=6.94 Hz, 4H), 3.74 (s, 4H), 2.38 (q, J=7.0 Hz, 4H), 2.31 (m, 8H), 2.13-1.96 (m, 4H), 1.65-1.59 (m, 8H), 1.42 (s, 6H), 1.38-1.23 (m, 16H), 0.96-0.82 (m, 6H).

### Step 3:

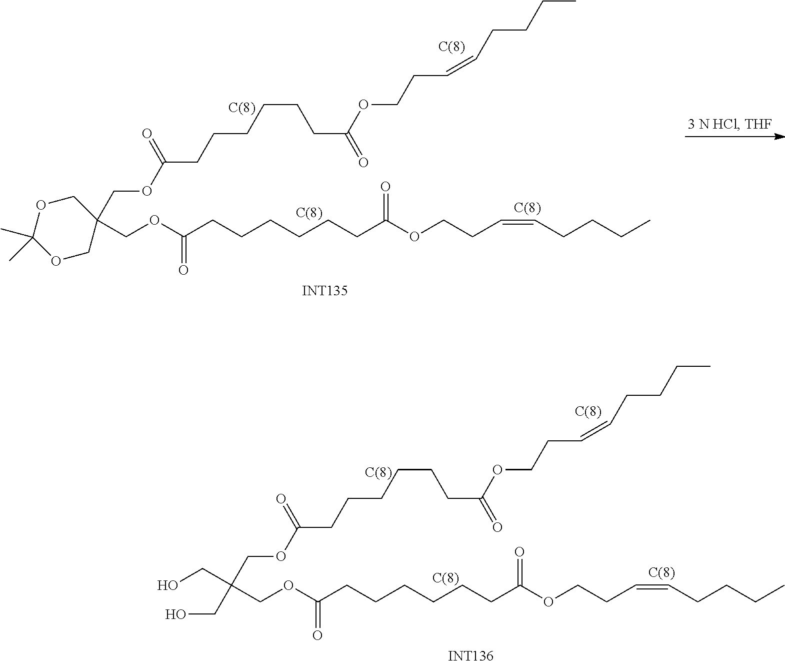 [SMILES: CCCC/C=C\CCOC(=O)CCCCCCC(=O)OCC1(COC(=O)CCCCCCC(=O)OCC/C=C\CCCC)COC(C)(C)OC1 ; "INT135" ; CCCC/C=C\CCOC(=O)CCCCCCC(=O)OCC(CO)(CO)COC(=O)CCCCCCC(=O)OCC/C=C\CCCC ; "INT136"]

To a solution of O8-[[2,2-dimethyl-5-[[8-[(Z)-oct-3-enoxy]-8-oxo-octanoyl]oxymethyl]-1,3-dioxan-5-yl]methyl]O1-[(Z)-oct-3-enyl]octanedioate (3.5 g, 4.94 mmol, 1 eq) in THF (35 mL) was added HCl (3 M, 1.65 mL, 1 eq) at 0° C. under N₂. The reaction mixture was stirred at 25° C. for 2 hr under N₂. The reaction mixture was diluted with sat. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis(hydroxymethyl)-3-[8-[(Z)-oct-3-enoxy]-8-oxo-octanoyl]oxy-propyl]O1-[(Z)-oct-3-enyl]octanedioate (1.2 g, 1.79 mmol, 36.34% yield) was obtained as a colorless oil. ¹H NMR (400 MHz, CDCl₃) δ ppm 5.52-5.50 (m, 2H), 5.36-5.33 (m, 2H), 4.15 (s, 4H), 4.07 (t, J=7.0 Hz, 4H), 3.59 (s, 4H), 2.75 (br s, 2H), 2.47-2.23 (m, 12H), 2.12-1.96 (m, 4H), 1.64 (m, 8H), 1.44-1.28 (m, 16H), 1.02-0.71 (m, 6H).

### Step 4:

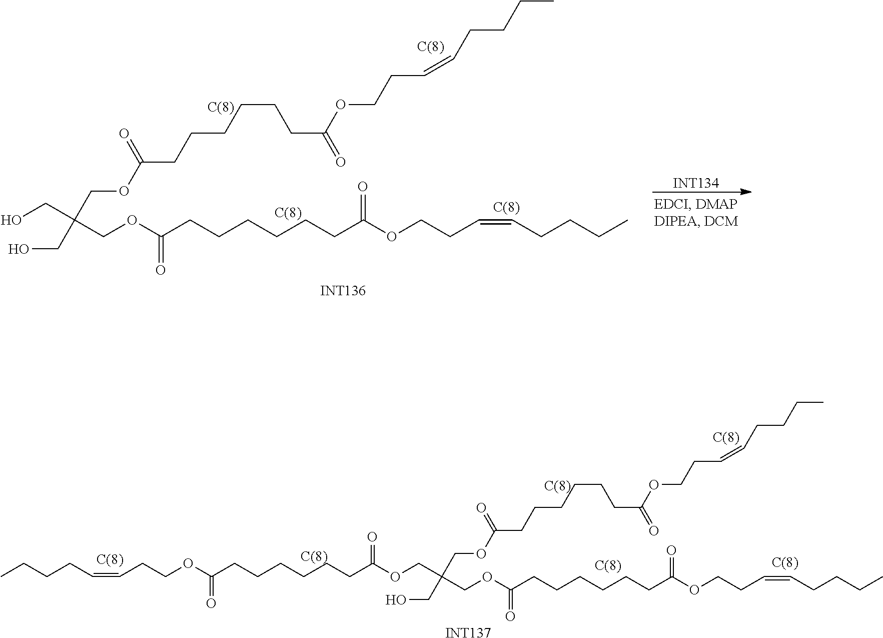

To a mixture of O8-[2,2-bis(hydroxymethyl)-3-[8-[(Z)-oct-3-enoxy]-8-oxo-octanoyl]oxy-propyl]O1-[(Z)-oct-3-enyl]octanedioate (1.2 g, 1.79 mmol, 1 eq), DMAP (21.92 mg, 179.40 mol, 0.1 eq), EDCI (412.69 mg, 2.15 mmol, 1.2 eq) and DIPEA (579.66 mg, 4.49 mmol, 781.21 μL, 2.5 eq) was added 8-[(Z)-oct-3-enoxy]-8-oxo-octanoic acid (306.12 mg, 1.08 mmol, 0.6 eq) under N₂. The reaction mixture was stirred at 25° C. for 12 hr under N₂. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2-(hydroxymethyl)-3-[8-[(Z)-oct-3-enoxy]-8-oxo-octanoyl]oxy-2-[[8-[(Z)-oct-3-enoxy]-8-oxo-octanoyl]oxymethyl]propyl]O1-[(Z)-oct-3-enyl]octanedioate (500 mg, 534.60 mol, 29.80% yield) was obtained as a colorless oil. ¹H NMR (400 MHz, CDCl₃) δ ppm 5.54-5.48 (m, 3H), 5.38-5.33 (m, 3H), 4.11 (s, 6H), 4.07 (t, J=6.8 Hz, 6 H), 3.50 (d, J=6.8 Hz, 2H), 2.65-2.57 (m, 1H), 2.43-2.28 (m, 18H), 2.05 (q, J=6.6 Hz, 6H), 1.71-1.59 (m, 12H), 1.41-1.29 (m, 24H), 1.00-0.82 (m, 9H).

### Step 5:

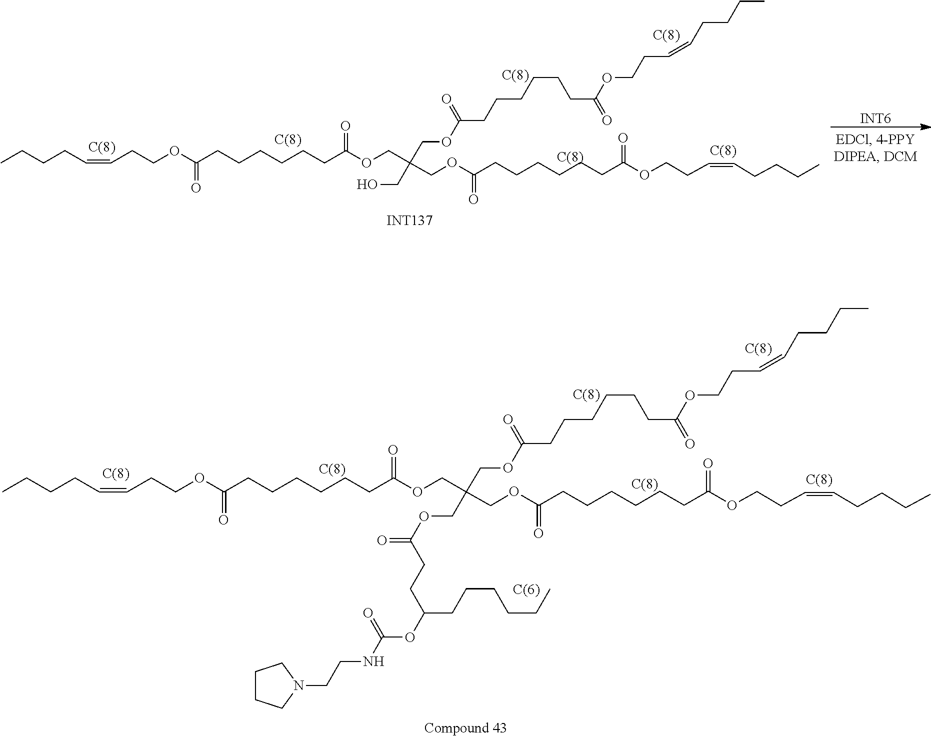

To a solution of O8-[2-(hydroxymethyl)-3-[8-[(Z)-oct-3-enoxy]-8-oxo-octanoyl]oxy-2-[[8-[(Z)-oct-3-enoxy]-8-oxo-octanoyl]oxymethyl]propyl]O1-[(Z)-oct-3-enyl]octanedioate (0.7 g, 748.45 mol, 1 eq) in DCM (10 mL) was added 4-(2-pyrrolidin-1-ylethylcarbamoyloxy) decanoic acid (294.99 mg, 898.14 mol, 1.2 eq), DIPEA (241.83 mg, 1.87 mmol, 325.91 μL, 2.5 eq), 4-pyrrolidin-1-ylpyridine (11.09 mg, 74.84 mol, 0.1 eq) and EDCI (172.17 mg, 898.14 mol, 1.2 eq) under N₂. The reaction mixture was stirred at 25° C. for 12 hr under N₂. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O8-[2,2-bis[[8-[(Z)-oct-3-enoxy]-8-oxo-octanoyl]oxymethyl]-3-[4-(2-pyrrolidin-1-ylethylcarbamoyloxy)decanoyloxy]propyl]O1-[(Z)-oct-3-enyl]octanedioate (110 mg, 88.30 mol, 11.80% yield, 100% purity) was obtained as a colorless oil. ¹H NMR (400 MHz, CDCl₃) δ ppm 5.52-5.49 (m, 3H), 5.35-5.33 (m, 3H), 4.75 (s, 1H), 4.11 (s, 8H), 4.06 (t, J=3.6 Hz, 6H), 3.32 (d, J=2.4 Hz, 2H), 2.61 (d, J=12.6 Hz, 6H), 2.40-2.28 (m, 20H), 2.07-2.02 (m, 6H), 1.90-1.80 (m, 6H), 1.64-1.60 (m, 14H), 1.35-1.25 (m, 32H), 0.93-0.86 (m, 12H).

### Example 51—Compound 44

### O7-[2,2-bis[[7-[(Z)-oct-3-enoxy]-7-oxo-heptanoyl]oxymethyl]-3-[4-(2-pyrrolidin-1-ylethylcarbamoyloxy)decanoyloxy]propyl]O1-[(Z)-oct-3-enyl]heptanedioate

### Step 1:

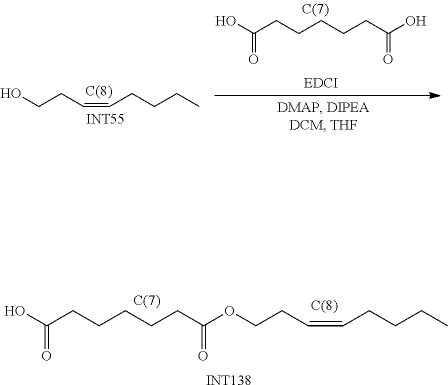

To a mixture of (Z)-oct-3-en-1-ol (20 g, 155.99 mmol, 1 eq), EDCI (35.88 g, 187.19 mmol, 1.2 eq), DMAP (1.91 g, 15.60 mmol, 0.1 eq) and DIPEA (50.40 g, 389.98 mmol, 67.93 mL, 2.5 eq) in DCM (700 mL) and THF (700 mL) was added heptanedioic acid (124.92 g, 779.96 mmol, 5 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 7-[(Z)-oct-3-enoxy]-7-oxo-heptanoic acid (30 g, 110.96 mmol, 71.13% yield) was obtained as a colorless oil. ¹H NMR (400 MHz, CDCl₃) δ ppm 11.00 (br s, 1H), 5.59-5.44 (m, 1H), 5.41-5.26 (m, 1H), 4.07 (t, J=6.2 Hz, 2H), 2.45-2.23 (m, 6H), 2.11-1.97 (m, 2H), 1.66 (m, 4H), 1.48-1.23 (m, 6H), 1.00-0.82 (m, 3H).

### Step 2:

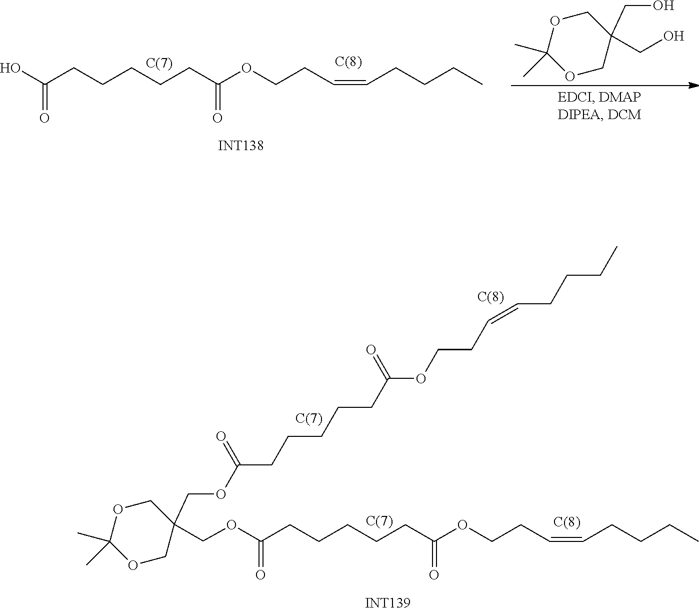

To a mixture of 7-[(Z)-non-3-enoxy]-7-oxo-heptanoic acid (17.93 g, 63.06 mmol, 1 eq), DMAP (770.34 mg, 6.31 mmol, 0.1 eq), EDCI (12.09 g, 63.06 mmol, 1 eq) and DIPEA (8.15 g, 63.06 mmol, 10.98 mL, 1 eq) in DCM (300 mL) was added [5-(hydroxymethyl)-2,2-dimethyl-1,3-dioxan-5-yl]methanol (5 g, 28.38 mmol, 0.45 eq) under N₂ atmosphere. The reaction mixture was stirred at 25° C. for 12 hr under N₂ atmosphere. The reaction mixture was diluted with aq. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[[2,2-dimethyl-5-[[7-[(Z)-non-3-enoxy]-7-oxo-heptanoyl]oxymethyl]-1,3-dioxan-5-yl]methyl]O1-[(Z)-non-3-enyl]heptanedioate (10 g, 14.11 mmol, 22.37% yield) was obtained as a colorless oil. ¹H NMR (400 MHz, CDCl₃) δ ppm 5.53-5.47 (m, 2H), 5.35-5.30 (m, 2H), 4.10-4.04 (m, 8H), 3.74 (s, 4H), 2.61-2.32 (m, 12H), 2.05 (br d, J=6.6 Hz, 4H), 1.64 (t, J=7.6 Hz, 8H), 1.42 (s, 6H), 1.39-1.21 (m, 12H), 0.90 (br t, J=6.4 Hz, 6H).

### Step 3:

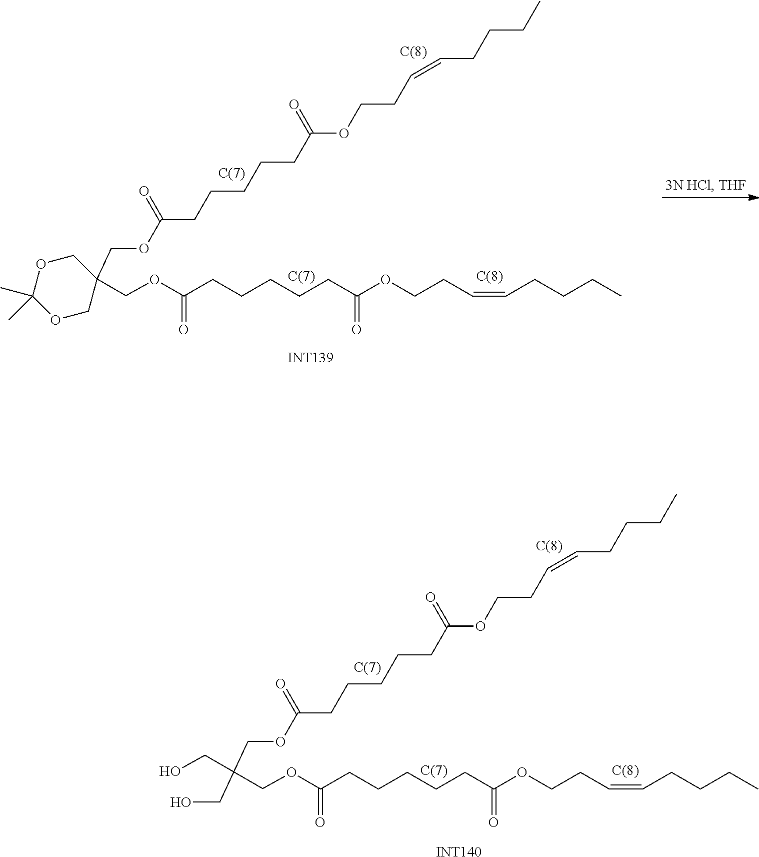 [SMILES: CCCC/C=C\CCOC(=O)CCCCCC(=O)OCC1(COC(=O)CCCCCC(=O)OCC/C=C\CCCC)COC(C)(C)OC1 ; "INT139" ; CCCC/C=C\CCOC(=O)CCCCCC(=O)OCC(CO)(CO)COC(=O)CCCCCC(=O)OCC/C=C\CCCC ; "INT140"]

To a solution of O7-[[2,2-dimethyl-5-[[7-[(Z)-non-3-enoxy]-7-oxo-heptanoyl]oxymethyl]-1,3-dioxan-5-yl]methyl]O1-[(Z)-non-3-enyl]heptanedioate (10 g, 14.11 mmol, 1 eq) in THF (100 mL) was added HCl (3 M, 5.17 mL, 1.1 eq) at 0° C. under N₂ atmosphere. The mixture was stirred at 25° C. for 12 hr under N₂ atmosphere. The reaction mixture was diluted with sat. NaHCO₃ (100 mL) and extracted with EtOAc 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis(hydroxymethyl)-3-[7-[(Z)-non-3-enoxy]-7-oxo-heptanoyl]oxy-propyl]O1-[(Z)-non-3-enyl]heptanedioate (5 g, 7.48 mmol, 52.99% yield) was obtained as a colorless oil.

### Step 4:

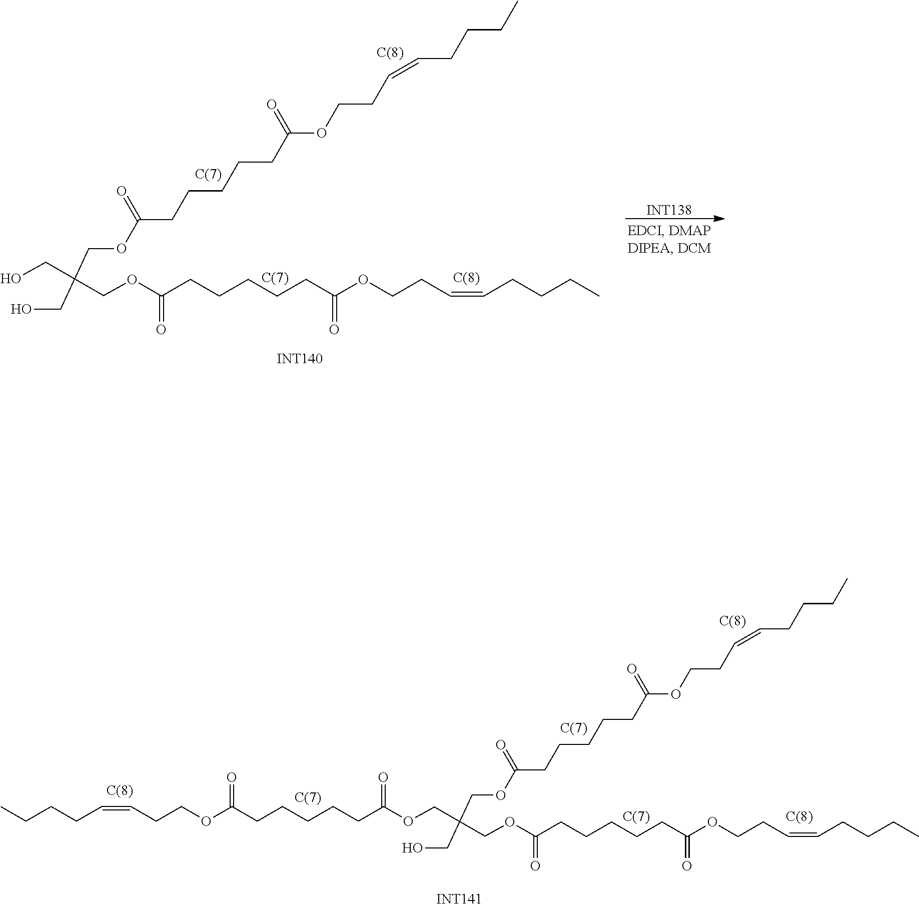

To a mixture of O7-[2,2-bis(hydroxymethyl)-3-[7-[(Z)-oct-3-enoxy]-7-oxo-heptanoyl]oxy-propyl]O1-[(Z)-oct-3-enyl]heptanedioate (2 g, 3.12 mmol, 1 eq), DIPEA (1.01 g, 7.80 mmol, 1.36 mL, 2.5 eq), EDCI (717.93 mg, 3.75 mmol, 1.2 eq) and DMAP (38.13 mg, 312.09 mol, 0.1 eq) in DCM (30 mL) was added 7-[(Z)-oct-3-enoxy]-7-oxo-heptanoic acid (506.27 mg, 1.87 mmol, 0.6 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2-(hydroxymethyl)-3-[7-[(Z)-oct-3-enoxy]-7-oxo-heptanoyl]oxy-2-[[7-[(Z)-oct-3-enoxy]-7oxoheptanoyl]oxymethyl]propyl]O1-[(Z)-oct-3-enyl]heptanedioate (0.8 g, 895.67 mol, 28.70% yield) was obtained as a colorless oil. ¹H NMR (400 MHz, CDCl₃) δ ppm 5.51-5.48 (m, 3H), 5.34-5.30 (m, 3H), 4.10-4.04 (m, 12H), 3.51 (d, J=3.2 Hz, 2H), 2.67 (t, J=3.2 Hz, 2H), 2.39-2.28 (m, 12H), 2.07-2.02 (m, 6H), 1.68-1.59 (m, 12H), 1.42-1.30 (m, 18H), 0.98-0.83 (m, 9H).

### Step 5:

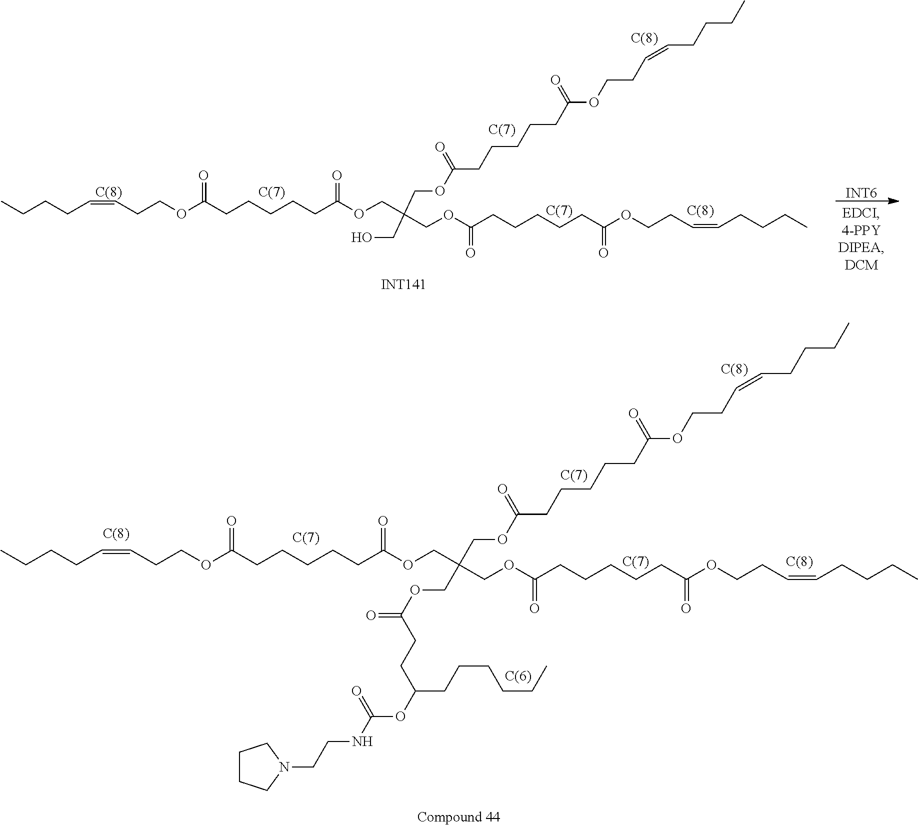

To a solution of O7-[2-(hydroxymethyl)-3-[7-[(Z)-oct-3-enoxy]-7-oxo-heptanoyl]oxy-2-[[7-[(Z)-oct-3-enoxy]-7-oxo-heptanoyl]oxymethyl]propyl]O1-[(Z)-oct-3-enyl]heptanedioate (1 g, 1.12 mmol, 1 eq) in DCM (15 mL) was added 4-(2-pyrrolidin-1-ylethylcarbamoyloxy)decanoic acid (330.95 mg, 1.01 mmol, 0.9 eq, DIPEA (361.75 mg, 2.80 mmol, 487.53 μL, 2.5 eq), 4-pyrrolidin-1-ylpyridine (16.59 mg, 111.96 mol, 0.1 eq) and EDCI (257.55 mg, 1.34 mmol, 1.2 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[[7-[(Z)-oct-3-enoxy]-7-oxo-heptanoyl]oxymethyl]-3-[4-(2-pyrrolidin-1-ylethylcarbamoyloxy)decanoyloxy]propyl]O1-[(Z)-oct-3-enyl]heptanedioate (110 mg, 91.39 mol, 8.16% yield, 100% purity) was obtained as a colorless oil. ¹H NMR (400 MHz, CDCl3) δ ppm 5.52-5.49 (m, 3H), 5.36-5.33 (m, 3H), 4.75 (s, 1H), 4.14-4.05 (m, 14H), 3.34 (s, 2H), 2.64 (d, J=10.0 Hz, 4H), 2.40-2.29 (m, 20H), 2.07-2.03 (m, 6H), 1.90-1.77 (m, 6H), 1.66-1.59 (m, 12H), 1.56-1.46 (m, 2H), 1.39-1.24 (m, 26H), 0.95-0.86 (m, 12H).

### Example 52—Compound 45

### O7-[2,2-bis[[7-[(Z)-dec-4-enoxy]-7-oxo-heptanoyl]oxymethyl]-3-[4-(2-pyrrolidin-1ylethylcarbamoyloxy)decanoyloxy]propyl]O1-[(Z)-dec-4-enyl]heptanedioate

### Step 1:

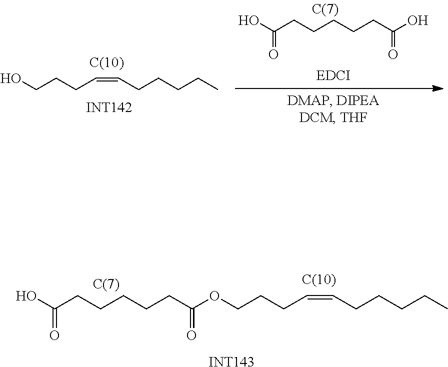

To a mixture of (Z)-dec-4-en-1-ol (20 g, 127.99 mmol, 1 eq), DMAP (1.56 g, 12.80 mmol, 0.1 eq), EDCI (29.44 g, 153.59 mmol, 1.2 eq) and DIPEA (41.35 g, 319.97 mmol, 55.73 mL, 2.5 eq) in DCM (600 mL) and THF (600 mL) was added heptanedioic acid (102.50 g, 639.94 mmol, 5 eq) under N₂ atmosphere. The reaction mixture was stirred at 25° C. for 12 hr under N₂ atmosphere. The reaction mixture was diluted with H₂O (2000 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 7-[(Z)-dec-4-enoxy]-7-oxo-heptanoic acid (25 g, 83.78 mmol, 65.46% yield) was obtained as a colorless oil. ¹H NMR (400 MHz, CDCl₃) δ ppm 5.43-5.32 (m, 2H), 4.08 (t, J=3.2 Hz, 2H), 2.39-2.30 (m, 4H), 2.14-2.00 (m, 4H), 1.72-1.65 (m, 6H), 1.40-1.29 (m, 8H), 0.90 (t, J=3.4 Hz, 3H).

### Step 2:

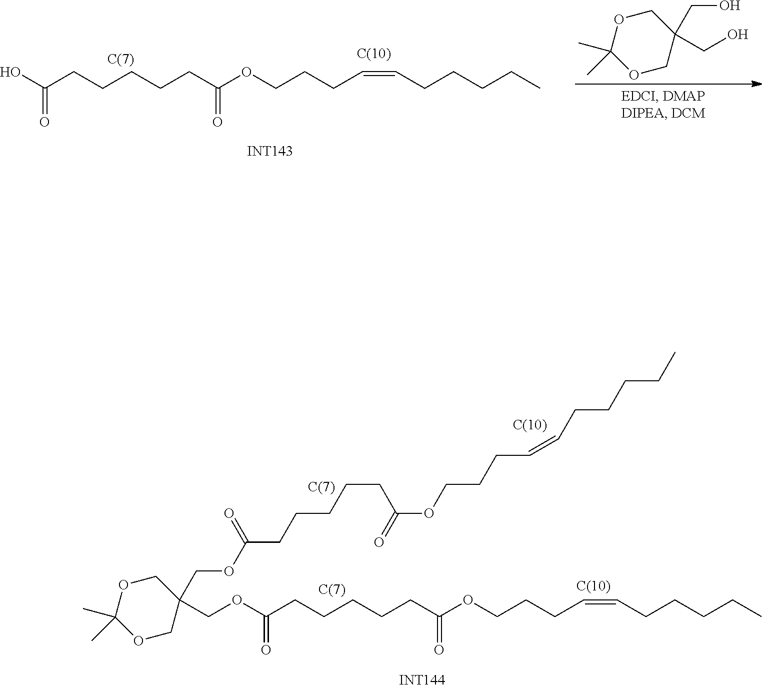

To a mixture of 7-[(Z)-dec-4-enoxy]-7-oxo-heptanoic acid (11.29 g, 37.83 mmol, 1 eq), DMAP (462.20 mg, 3.78 mmol, 0.1 eq), EDCI (8.70 g, 45.40 mmol, 1.2 eq) and DIPEA (12.22 g, 94.58 mmol, 16.47 mL, 2.5 eq) in DCM (150 mL) was added [5-(hydroxymethyl)-2,2-dimethyl-1,3-dioxan-5-yl]methanol (3 g, 17.03 mmol, 0.45 eq) under N₂ atmosphere. The reaction mixture was stirred at 25° C. for 12 hr under N₂ atmosphere. The reaction mixture was diluted with H₂O (150 mL) and extracted with DCM 450 mL (150 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[[5-[[7-[(Z)-dec-4-enoxy]-7-oxo-heptanoyl]oxymethyl]-2,2-dimethyl-1,3-dioxan-5-yl]methyl]O1-[(Z)-dec-4-enyl]heptanedioate (5.5 g, 7.46 mmol, 19.72% yield) was obtained as a colorless oil. ¹H NMR (400 MHz, CDCl₃) δ ppm 5.43-5.39 (m, 2H), 5.37-5.32 (m, 2H), 4.11 (s, 4H), 4.07 (t, J=6.6 Hz, 4H), 3.74 (s, 4H), 2.32 (q, J=7.8 Hz, 8H), 2.10 (q, J=7.2 Hz, 4H), 2.01 (q, J=7.0 Hz, 4H), 1.70-1.58 (m, 12H), 1.42 (s, 6H), 1.39-1.25 (m, 16H), 0.89 (t, J=6.8 Hz, 6H).

### Step 3:

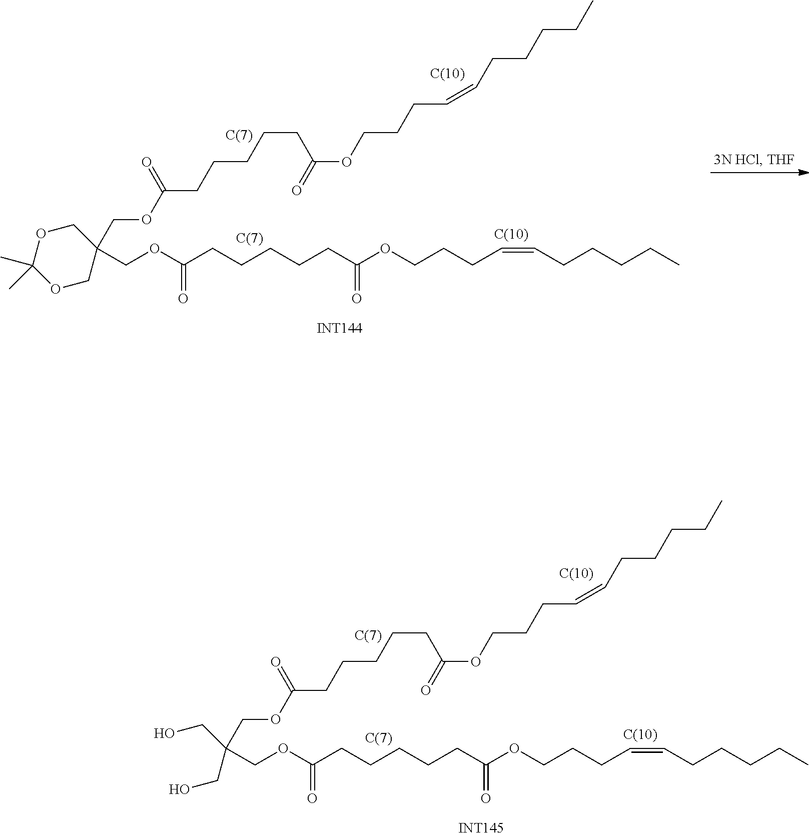 [SMILES: CCCCC/C=C\CCCOC(=O)CCCCCC(=O)OCC1(COC(=O)CCCCCC(=O)OCCC/C=C\CCCCC)COC(C)(C)OC1 ; "INT144" ; CCCCC/C=C\CCCOC(=O)CCCCCC(=O)OCC(CO)(CO)COC(=O)CCCCCC(=O)OCCC/C=C\CCCCC ; "INT145"]

To a solution of O7-[[5-[[7-[(Z)-dec-4-enoxy]-7-oxo-heptanoyl]oxymethyl]-2,2-dimethyl-1,3-dioxan-5-yl]methyl]O1-[(Z)-dec-4-enyl]heptanedioate (5.5 g, 7.46 mmol, 1 eq) in THF (55 mL) was added HCl (3 M, 2.74 mL, 1.1 eq) at 0° C. under N₂ atmosphere. The mixture was stirred at 25° C. for 12 hr under N₂ atmosphere. The reaction mixture was diluted with sat. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2-[[7-[(Z)-dec-4-enoxy]-7-oxo-heptanoyl]oxymethyl]-3-hydroxy-2-(hydroxymethyl)propyl]O1-[(Z)-dec-4-enyl]heptanedioate (2 g, 2.87 mmol, 38.45% yield) was obtained as a colorless oil. ¹H NMR (400 MHz, CDCl₃) δ ppm 5.43-5.38 (m, 2H), 5.38-5.32 (m, 2H), 4.14 (s, 4H), 4.07 (t, J=6.6 Hz, 4H), 3.59 (s, 4H), 2.42-2.30 (m, 8H), 2.10 (q, J=7.4 Hz, 4H), 2.06-1.99 (m, 4H), 1.66 (m, 12H), 1.40-1.26 (m, 16H), 0.89 (t, J=6.8 Hz, 6H).

### Step 4:

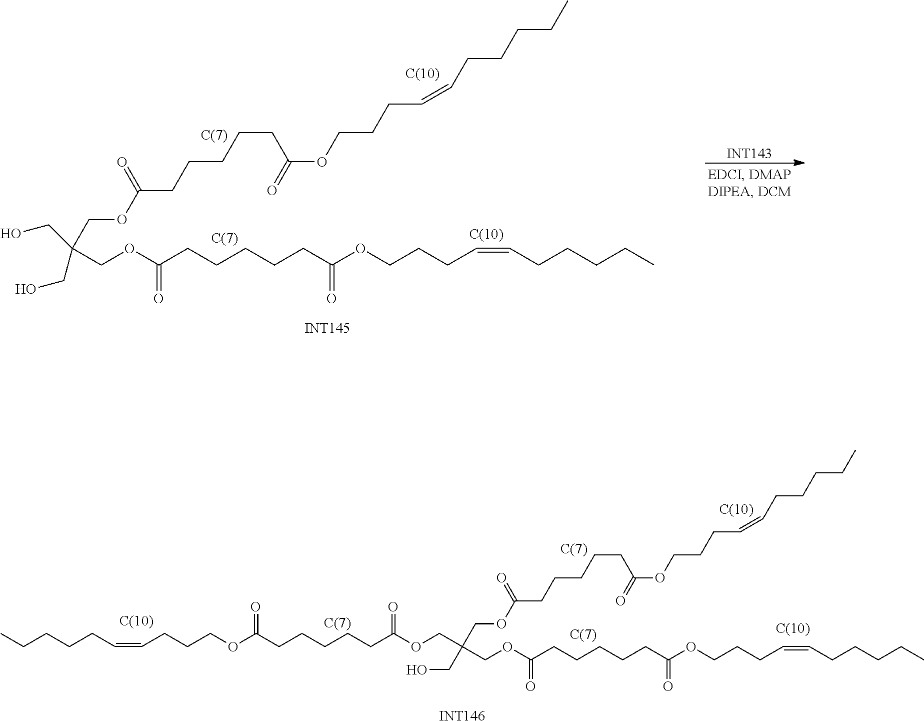

To a mixture of O7-[2-[[7-[(Z)-dec-4-enoxy]-7-oxo-heptanoyl]oxymethyl]-3-hydroxy-2-(hydroxymethyl)propyl]O1-[(Z)-dec-4-enyl]heptanedioate (2 g, 2.87 mmol, 1 eq), EDCI (660.14 mg, 3.44 mmol, 1.2 eq), DMAP (35.06 mg, 286.97 mol, 0.1 eq) and DIPEA (927.21 mg, 7.17 mmol, 1.25 mL, 2.5 eq) in DCM (25 mL) was added 7-[(Z)-dec-4-enoxy]-7-oxo-heptanoic acid (513.81 mg, 1.72 mmol, 0.6 eq) under N₂ atmosphere. The reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[[7-[(Z)-dec-4-enoxy]-7-oxo-heptanoyl]oxymethyl]-3-hydroxy-propyl]O1-[(Z)-dec-4-enyl]heptanedioate (0.8 g, 818.54 mol, 28.52% yield) was obtained as a colorless oil. ¹H NMR (400 MHz, CDCl₃) δ ppm 5.43-5.39 (m, 3H), 5.37-5.32 (m, 3H), 4.11 (s, 6H), 4.07 (t, J=6.6 Hz, 6H), 3.51 (s, 2H), 2.33 (m, 12H), 2.11 (q, J=7.2 Hz, 6H), 2.02 (q, J=7.0 Hz, 6H), 1.74-1.61 (m, 20H), 1.38-1.27 (m, 22H), 0.89 (t, J=6.8 Hz, 9H).

### Step 5:

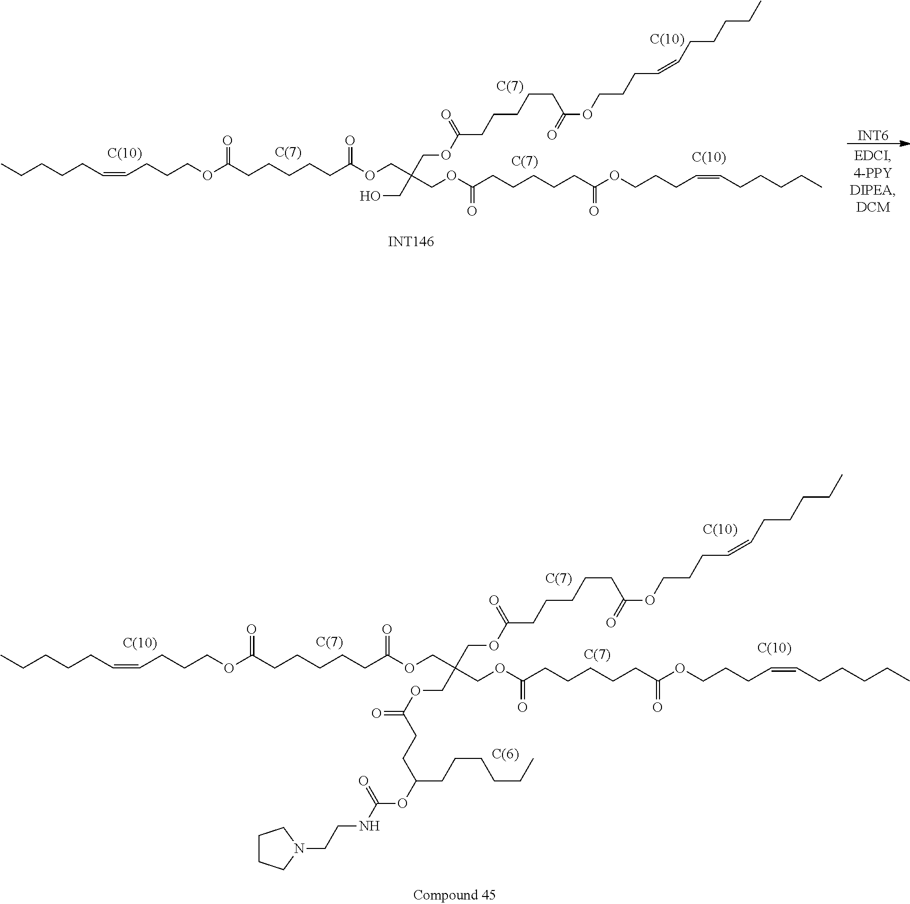

To a mixture of O7-[2,2-bis[[7-[(Z)-dec-4-enoxy]-7-oxo-heptanoyl]oxymethyl]-3-hydroxy-propyl]O1-[(Z)-dec-4-enyl]heptanedioate (0.8 g, 818.54 mol, 1 eq), 4-pyrrolidin-1-ylpyridine (12.13 mg, 81.85 mol, 0.1 eq), DIPEA (264.48 mg, 2.05 mmol, 356.44 L, 2.5 eq) and EDCI (188.30 mg, 982.25 mol, 1.2 eq) in DCM (11 mL) was added 4-(2-pyrrolidin-1-ylethylcarbamoyloxy)decanoic acid (322.62 mg, 982.25 mol, 1.2 eq), under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O7-[2,2-bis[[7-[(Z)-dec-4-enoxy]-7-oxo-heptanoyl]oxymethyl]-3-[4-(2-pyrrolidin-1ylethylcarbamoyloxy)decanoyloxy]propyl]O1-[(Z)-dec-4-enyl]heptanedioate (0.1 g, 77.65 mol, 9.49% yield, 100% purity) was obtained as a colorless oil. ¹H NMR (400 MHz, CDCl₃) δ ppm 5.96-5.75 (m, 1H), 5.43-5.32 (m, 6H), 4.74 (s, 1H), 4.11 (s, 8H), 4.07 (t, J=3.0 Hz, 6H), 3.47 (s, 2H), 3.03-2.80 (m, 6H), 2.34-2.30 (m, 14H), 2.13-2.08 (m, 6H), 2.04-2.00 (m, 8H), 1.70-1.61 (m, 26H), 1.36-1.26 (m, 30H), 1.35-1.25 (m, 32H), 0.91-0.86 (m, 12H).

### Example 53—Compound 46

### 8,8′-di((Z)-dec-4-en-1-yl) O′1,O1-(2-(((8-(((Z)-dec-4-en-1-yl)oxy)-8-oxooctanoyl)oxy)methyl)-2-(((4-(((2-(pyrrolidin-1-yl)ethyl)carbamoyl)oxy)decanoyl)oxy)methyl)propane-1,3-diyl) di(octanedioate)

### Step 1:

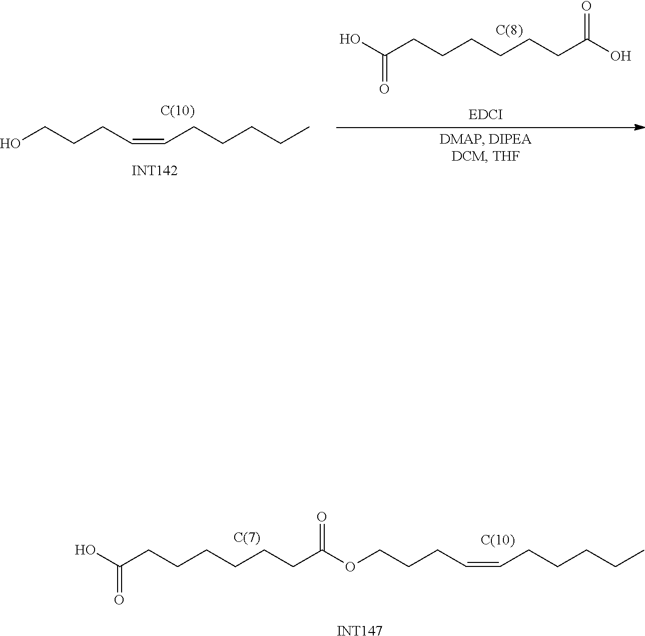

To a solution of (Z)-dec-4-en-1-ol (20 g, 127.99 mmol, 1 eq) in DCM (600 mL) and THF (600 mL) was added octanedioic acid (111.47 g, 639.94 mmol, 5 eq), EDCI (31.90 g, 166.38 mmol, 1.3 eq), DIPEA (49.62 g, 383.96 mmol, 66.88 mL, 3 eq) and DMAP (3.13 g, 25.60 mmol, 0.2 eq) under N₂ atmosphere. The mixture was stirred at 20° C. for 12 hr under N₂ atmosphere. The reaction mixture was poured into H₂O (500 mL), and extracted with DCM (500 mL×2). The combined organic layers were washed with brine (300 mL×2), dried over Na₂SO₄, filtered and concentrated under reduced pressure to give a residue. The residue was purified by column chromatography (SiO₂, Petroleum ether/Ethyl acetate=5/1 to 3/1). Compound 8-[(Z)-dec-4-enoxy]-8-oxo-octanoic acid (22 g, 70.41 mmol, 55.01% yield) was obtained as a colorless oil. ¹H NMR (400 MHz, CDCl₃) δ ppm 11.07 (br s, 1H), 5.49-5.25 (m, 2H), 4.07 (t, J=6.6 Hz, 2H), 2.33 (m, 4H), 2.11 (q, J=7.4 Hz, 2H), 2.02 (q, J=7.0 Hz, 2H), 1.75-1.58 (m, 6H), 1.41-1.19 (m, 11H), 0.89 (t, J=6.8 Hz, 3H).

### Step 2:

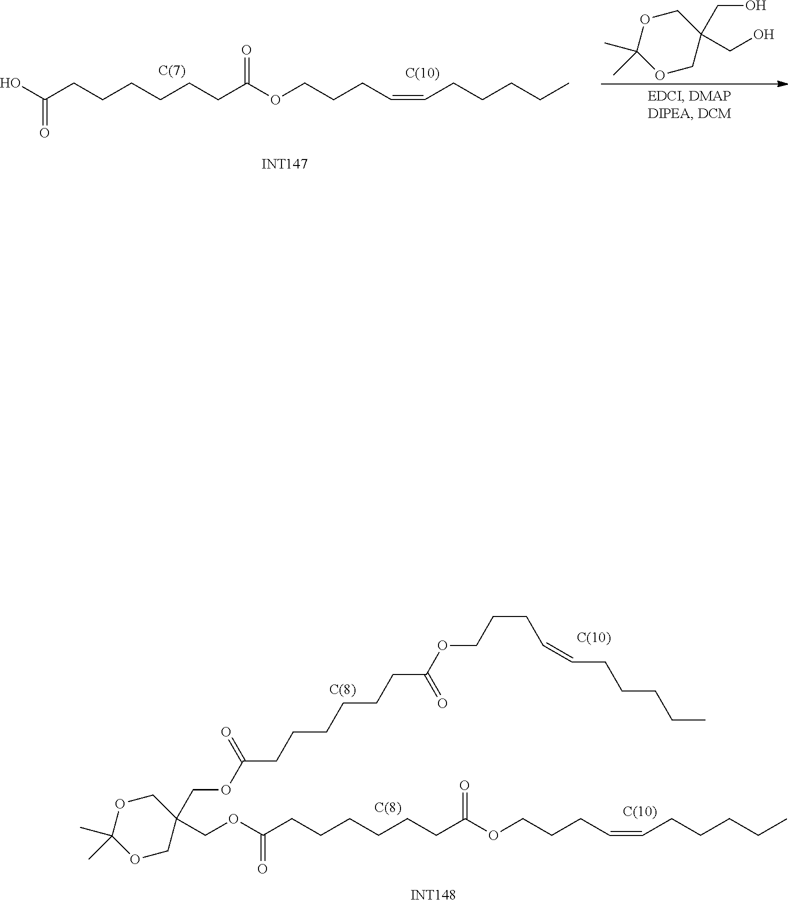

To a solution of 8-[(Z)-dec-4-enoxy]-8-oxo-octanoic acid (10.64 g, 34.05 mmol, 1 eq) and [5-(hydroxymethyl)-2,2-dimethyl-1,3-dioxan-5-yl]methanol (3 g, 17.03 mmol, 0.5 eq) in DCM (130 mL) was added DIEA (11.00 g, 85.13 mmol, 14.83 mL, 2.5 eq), EDCI (7.83 g, 40.86 mmol, 1.2 eq) and DMAP (415.98 mg, 3.41 mmol, 0.1 eq) under N₂ atmosphere. The reaction mixture was stirred at 20° C. for 12 hr under N₂ atmosphere. The reaction mixture was diluted into H₂O (100 mL) and extracted with DCM (100 mL×2). The combined organic layers were washed with brine (300 mL×2), dried over Na₂SO₄, filtered and the filtrate was concentrated under reduced pressure to give a residue. The residue was purified by column chromatography (SiO₂, Petroleum ether/Ethyl acetate=14/1 to 10/1). Compound O8-[[5-[[8-[(Z)-dec-4-enoxy]-8-oxo-octanoyl]oxymethyl]-2,2-dimethyl-1,3-dioxan-5-yl]-methyl]O1-[(Z)-dec-4-enyl]octanedioate (9 g, 11.76 mmol, 68.97% yield) was obtained as a colorless oil. ¹H NMR (400 MHz, CDCl₃) δ ppm 5.41-5.37 (m, 2H), 5.35-5.31 (m, 2H), 4.09 (s, 4H), 4.05 (t, J=6.6 Hz, 4H), 3.72 (s, 4H), 2.29 (q, J=7.6 Hz, 8H), 2.09 (q, J=7.2 Hz, 4H), 2.00 (q, J=7.0 Hz, 4H), 1.72-1.58 (m, 12H), 1.40 (s, 6H), 1.36-1.19 (m, 20H), 0.87 (t, J=6.8 Hz, 6H).

### Step 3:

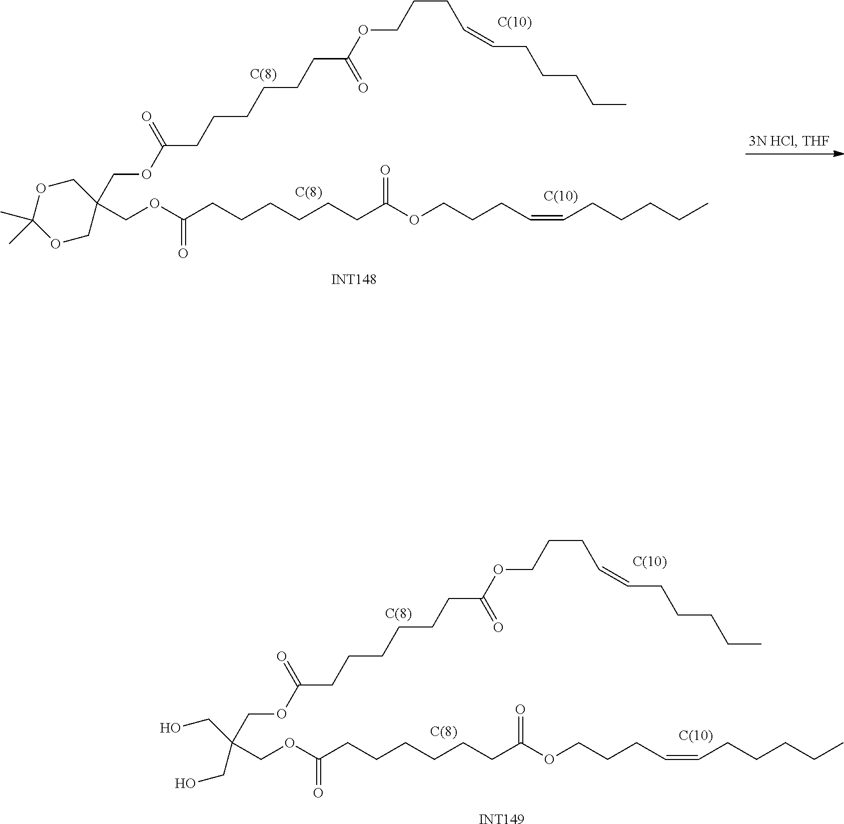 [SMILES: CCCCC/C=C\CCCOC(=O)CCCCCCC(=O)OCC1(COC(=O)CCCCCCC(=O)OCCC/C=C\CCCCC)COC(C)(C)OC1 ; "INT148" ; CCCCC/C=C\CCCOC(=O)CCCCCCC(=O)OCC(CO)(CO)COC(=O)CCCCCCC(=O)OCCC/C=C\CCCCC ; "INT149"]

To a solution of O8-[[5-[[8-[(Z)-dec-4-enoxy]-8-oxo-octanoyl]oxymethyl]-2,2-dimethyl-1,3-dioxan-5-yl]methyl]O1-[(Z)-dec-4-enyl]octanedioate (3 g, 3.92 mmol, 1 eq) in THF (100 mL) was added HCl (3 M, 1.57 mL, 1.2 eq) at 0° C. under N₂ atmosphere. The mixture was stirred at 20° C. for 12 hr under N₂ atmosphere. The reaction mixture was diluted with sat. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=8/1 to 10/1). Compound O′1,O1-(2,2-bis(hydroxymethyl)propane-1,3-diyl) 8,8′-di((Z)-dec-4-en-1-yl) di(octanedioate) (3 g, 4.30 mmol, 35.25% yield) was obtained as a colorless oil.

### Step 4:

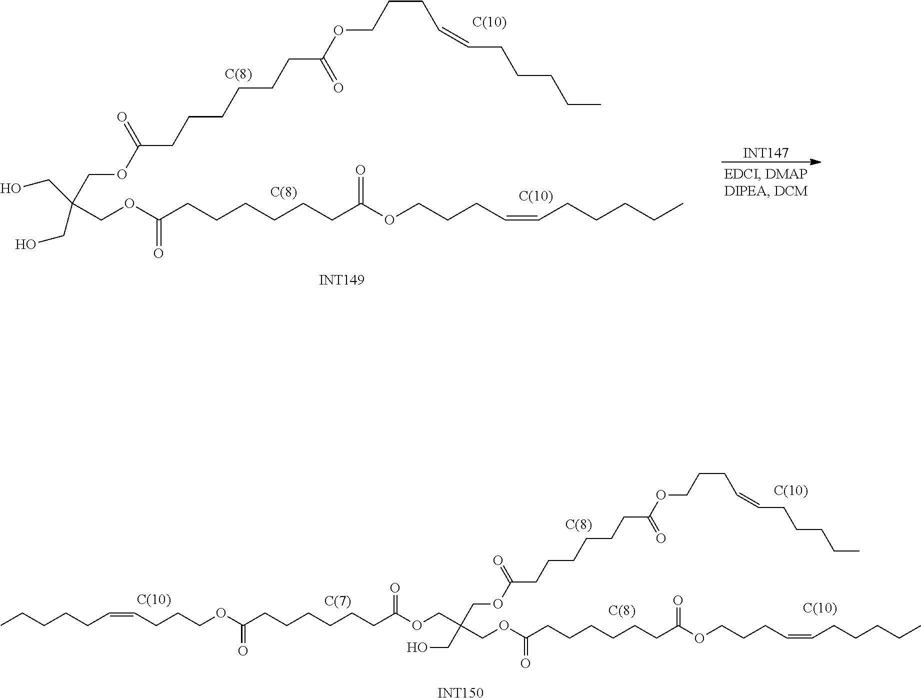

To a solution of 7-[(Z)-dec-4-enoxy]-7-oxo-heptanoic acid (770.72 mg, 2.58 mmol, 0.6 eq) in DCM (40 mL) was added DIEA (1.39 g, 10.76 mmol, 1.87 mL, 2.5 eq), EDCI (990.21 mg, 5.17 mmol, 1.2 eq) and DMAP (52.59 mg, 430.45 mol, 0.1 eq) under N₂ atmosphere. Then O′1,O1-(2,2-bis(hydroxymethyl)propane-1,3-diyl) 8,8′-di((Z)-dec-4-en-1-yl) di(octanedioate) (3 g, 4.30 mmol, 1 eq) was added to the above reaction mixture under N₂ atmosphere and the reaction mixture was stirred at 20° C. for 12 hr under N₂ atmosphere. The reaction mixture was diluted with H₂O (50 mL) and extracted with DCM (50 mL×3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=8/1 to 10/1). Compound 8,8′-di((Z)-dec-4-en-1-yl) O′1,O1-(2-(((8-(((Z)-dec-4-en-1-yl)oxy)-8-oxooctanoyl)oxy)methyl)-2-(hydroxymethyl)propane-1,3-diyl) di(octanedioate) (1.4 g, 1.43 mmol, 33.25% yield) was obtained as a colorless oil. ¹H NMR (400 MHz, CDCl₃) δ ppm 5.42-5.37 (m, 3H), 5.36-5.30 (m, 3H), 4.10 (s, 6H), 4.06 (t, J=6.6 Hz, 6H), 3.50 (br d, J=5.0 Hz, 2H), 2.71-2.59 (m, 1H), 2.31 (m, 12H), 2.10 (q, J=7.4 Hz, 6H), 2.01 (q, J=7.0 Hz, 6H), 1.73-1.57 (m, 18H), 1.41-1.21 (m, 30H), 0.88 (t, J=6.8 Hz, 9H).

### Step 5:

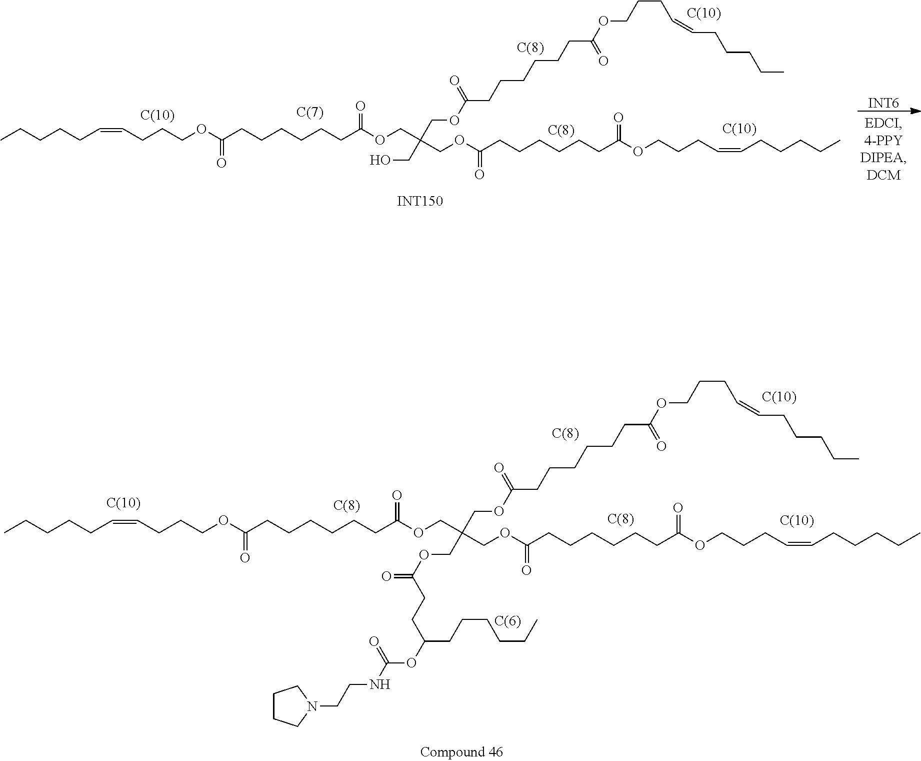

To a solution of 8,8′-di((Z)-dec-4-en-1-yl) O′1,O1-(2-(((8-(((Z)-dec-4-en-1-yl)oxy)-8-oxooctanoyl)oxy)-methyl)-2-(hydroxymethyl)propane-1,3-diyl)di(octanedioate) (800 mg, 818.54 mol, 1 eq) and 4-(2-pyrrolidin-1-ylethylcarbamoyloxy)decanoic acid (403.27 mg, 1.23 mmol, 1.5 eq) in DCM (10 mL) was added DIEA (264.48 mg, 2.05 mmol, 356.44 L, 2.5 eq), EDCI (188.30 mg, 982.25 mol, 1.2 eq) and 4-pyrrolidin-1-ylpyridine (12.13 mg, 81.85 mol, 0.1 eq) under N₂ atmosphere. The reaction mixture was stirred at 20° C. for 12 hr under N₂ atmosphere. The reaction mixture was concentrated under reduced pressure to remove solvent. The residue was diluted with H₂O (30 mL) and extracted with DCM 90 mL (30 mL*3). The combined organic layers were washed with brine (50 mL×2), dried over Na₂SO₄, filtered and the filtrate was concentrated under reduced pressure to give a residue. The residue was purified by column chromatography (SiO₂, Petroleum ether/Ethyl acetate=100/1 to 0/1). Compound 8,8′-di((Z)-dec-4-en-1-yl) O′1,O1-(2-(((8-(((Z)-dec-4-en-1-yl)oxy)-8-oxooctanoyl)oxy)methyl)-2-(((4-(((2-(pyrrolidin-1-yl)ethyl)carbamoyl)oxy)decanoyl)oxy)methyl)propane-1,3-diyl) di(octanedioate) (0.1 g, 75.20 mol, 5.11% yield) was obtained as a colorless oil. ¹H NMR (400 MHz, CDCl₃) δ ppm 5.43-5.39 (m, 3H), 5.38-5.32 (m, 3H), 4.75 (br s, 1H), 4.25-3.93 (m, 14H), 3.37 (br s, 2H), 2.50-2.78 (m, 4H), 2.44-2.28 (m, 14H), 2.11 (q, J=7.2 Hz, 6H), 2.02 (q, J=7.0 Hz, 6H), 1.94-1.74 (m, 6H), 1.73-1.55 (m, 22H), 1.39-1.22 (m, 40H), 0.93-0.85 (m, 12H).

### Example 54—Compound 47

### O6-[2,2-bis[[6-[(Z)-dec-4-enoxy]-6-oxo-hexanoyl]oxymethyl]-3-[4-(2-pyrrolidin-1ylethylcarbamoyloxy)decanoyloxy]propyl]O1-[(Z)-dec-4-enyl]hexanedioate

### Step 1:

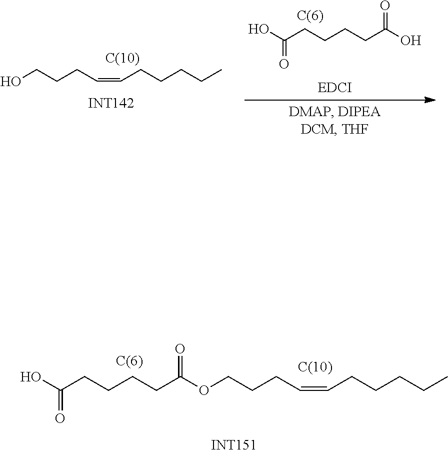

To a mixture of (Z)-dec-4-en-1-ol (20 g, 127.99 mmol, 1 eq), DMAP (1.56 g, 12.80 mmol, 0.1 eq), EDCI (29.44 g, 153.59 mmol, 1.2 eq) and DIPEA (41.35 g, 319.97 mmol, 55.73 mL, 2.5 eq) in DCM (500 mL) and THF (500 mL) was added adipic acid (93.52 g, 639.94 mmol, 106.27 mL, 5 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 6-[(Z)-dec-4-enoxy]-6-oxo-hexanoic acid (25 g, 87.91 mmol, 68.68% yield) was obtained as a colorless oil. ¹H NMR (400 MHz, CDCl₃) δ ppm 11.96-10.46 (m, 2H), 5.43-5.31 (m, 2H), 4.07 (t, J=3.2 Hz, 2H), 2.40-2.32 (m, 4H), 2.13-1.99 (m, 4H), 1.73-1.65 (m, 6H), 1.38-1.25 (m, 6H), 0.89 (t, J=3.4 Hz, 3H).

### Step 2:

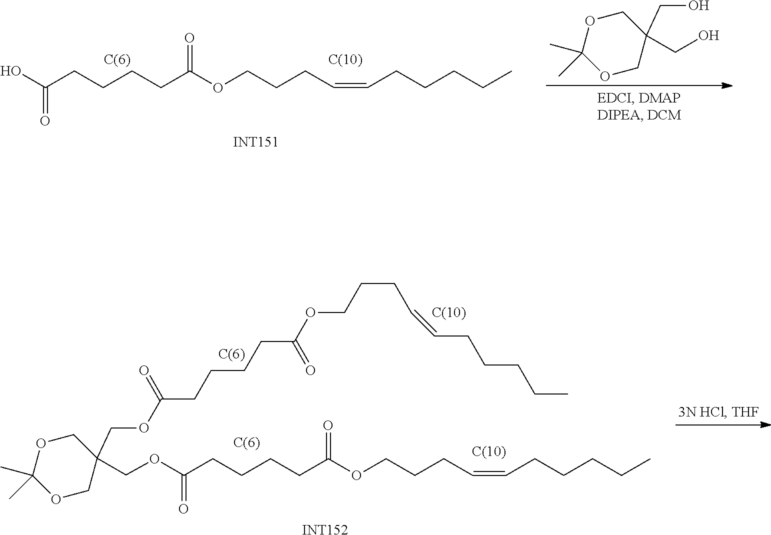

To a mixture of 6-[(Z)-dec-4-enoxy]-6-oxo-hexanoic acid (10.76 g, 37.83 mmol, 1 eq), DMAP (462.20 mg, 3.78 mmol, 0.1 eq), EDCI (8.70 g, 45.40 mmol, 1.2 eq) and DIPEA (12.22 g, 94.58 mmol, 16.47 mL, 2.5 eq) in DCM (130 mL) was added [5-(hydroxymethyl)-2,2-dimethyl-1,3-dioxan-5-yl]methanol (3 g, 17.03 mmol, 0.45 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[[5-[[6-[(Z)-dec-4-enoxy]-6-oxo-hexanoyl]oxymethyl]-2,2-dimethyl-1,3-dioxan-5-yl]methyl]O1-[(Z)-dec-4-enyl]hexanedioate (4 g, 5.64 mmol, 14.91% yield) was obtained as a colorless oil.

### Step 3:

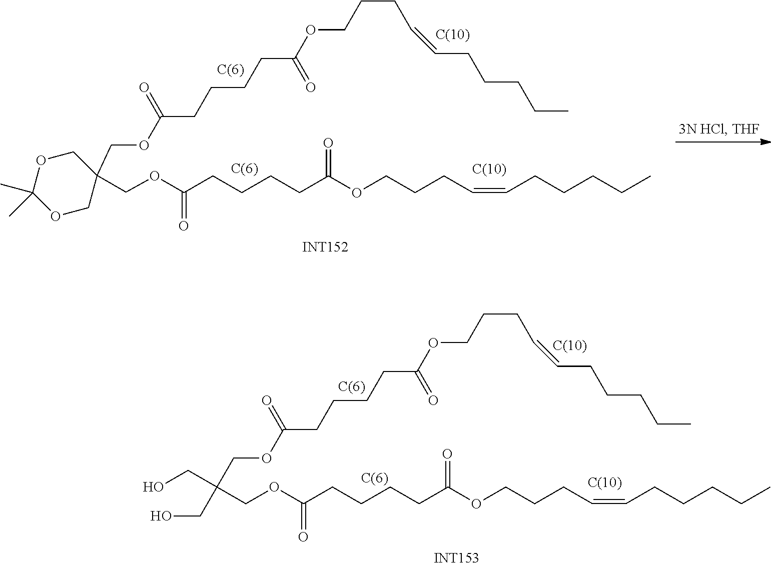 [SMILES: CCCCC/C=C\CCCOC(=O)CCCCC(=O)OCC1(COC(=O)CCCCC(=O)OCCC/C=C\CCCCC)COC(C)(C)OC1 ; "INT152" ; CCCCC/C=C\CCCOC(=O)CCCCC(=O)OCC(CO)(CO)COC(=O)CCCCC(=O)OCCC/C=C\CCCCC ; "INT153"]

To a solution of O6-[[5-[[6-[(Z)-dec-4-enoxy]-6-oxo-hexanoyl]oxymethyl]-2,2-dimethyl-1,3-dioxan-5-yl]methyl]O1-[(Z)-dec-4-enyl]hexanedioate (4 g, 5.64 mmol, 1 eq) in THF (40 mL) was added HCl (3 M, 1.88 mL, 1 eq) at 0° C. under N₂ atmosphere. The reaction mixture was stirred at 25° C. for 12 hr under N₂ atmosphere. The reaction mixture was diluted with sat. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[6-[(Z)-dec-4-enoxy]-6-oxo-hexanoyl]oxymethyl]-3-hydroxy-2-(hydroxymethyl)propyl]O1-[(Z)-dec-4-enyl]hexanedioate (1.4 g, 2.09 mmol, 37.10% yield) was obtained as a colorless oil. ¹H NMR (400 MHz, CDCl₃) δ ppm 5.43-5.38 (m, 2H), 5.37-5.32 (m, 2H), 4.15 (s, 4H), 4.08 (t, J=6.6 Hz, 4H), 3.59 (s, 4H), 2.77 (br s, 2H), 2.47-2.31 (m, 8H), 2.11 (q, J=7.4 Hz, 4H), 2.02 (q, J=7.0 Hz, 4H), 1.72-1.67 (m, 12H), 1.41-1.25 (m, 12H), 0.89 (t, J=6.8 Hz, 6H).

### Step 4:

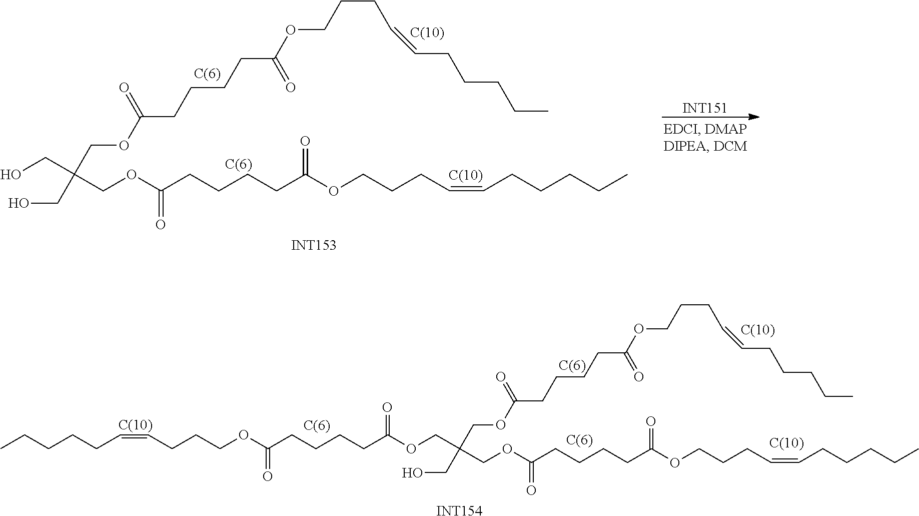

To a mixture of O6-[2-[[6-[(Z)-dec-4-enoxy]-6-oxo-hexanoyl]oxymethyl]-3-hydroxy-2-(hydroxymethyl)propyl]O1-[(Z)-dec-4-enyl]hexanedioate (1.4 g, 2.09 mmol, 1 eq), DMAP (25.57 mg, 209.30 mol, 0.1 eq), EDCI (481.48 mg, 2.51 mmol, 1.2 eq) and DIPEA (676.26 mg, 5.23 mmol, 911.41 L, 2.5 eq) in DCM (20 mL) was added 6-[(Z)-dec-4-enoxy]-6-oxo-hexanoic acid (357.14 mg, 1.26 mmol, 0.6 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2,2-bis[[6-[(Z)-dec-4-enoxy]-6-oxo-hexanoyl]oxymethyl]-3-hydroxy-propyl]O1-[(Z)-dec-4-enyl]hexanedioate (0.6 g, 641.53 mol, 30.65% yield) was obtained as a colorless oil. ¹H NMR (400 MHz, CDCl₃) δ ppm 5.43-5.38 (m, 3H), 5.37-5.32 (m, 3H), 4.11 (s, 6H), 4.07 (t, J=6.6 Hz, 6H), 3.52 (br s, 2H), 2.67 (br s, 1H), 2.35 (m, 12H), 2.10 (q, J=7.4 Hz, 6H), 2.02 (q, J=7.0 Hz, 6H), 1.71-1.61 (m, 18H), 1.37-1.18 (m, 18H), 0.89 (t, J=6.8 Hz, 9H).

### Step 5:

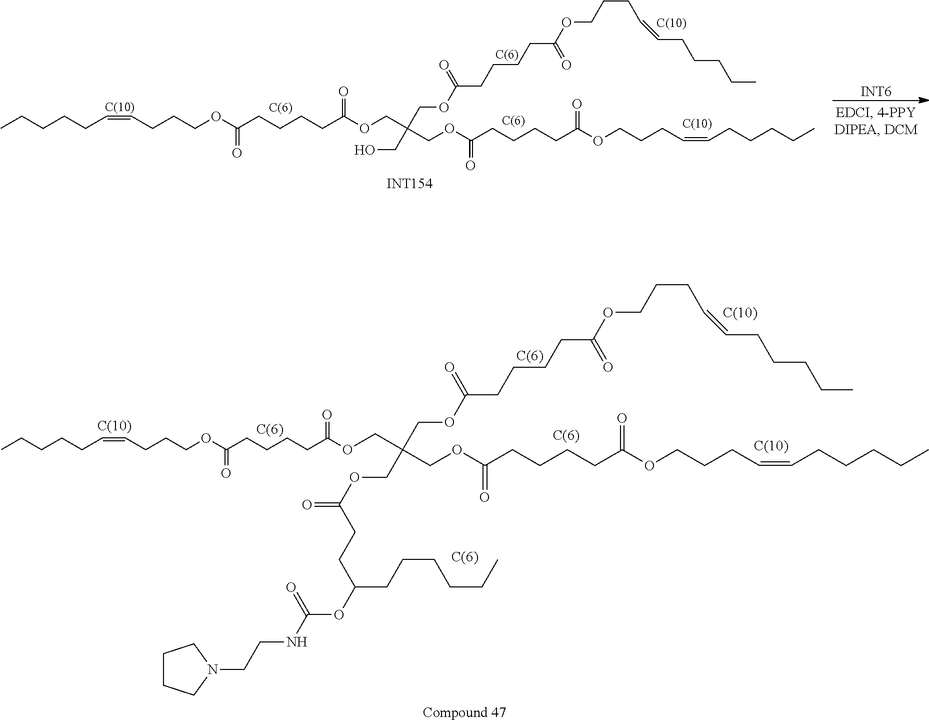

To a mixture of O6-[2,2-bis[[6-[(Z)-dec-4-enoxy]-6-oxo-hexanoyl]oxymethyl]-3-hydroxy-propyl]O1-[(Z)-dec-4-enyl]hexanedioate (0.6 g, 641.53 mol, 1 eq), 4-pyrrolidin-1-ylpyridine (9.51 mg, 64.15 mol, 0.1 eq), DIPEA (207.28 mg, 1.60 mmol, 279.36 L, 2.5 eq) and EDCI (147.58 mg, 769.83 mol, 1.2 eq) in DCM (9 mL) was added 4-(2-pyrrolidin-1-ylethylcarbamoyloxy)decanoic acid (252.85 mg, 769.83 mol, 1.2 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2,2-bis[[6-[(Z)-dec-4-enoxy]-6-oxo-hexanoyl]oxymethyl]-3-[4-(2-pyrrolidin-1ylethylcarbamoyloxy)decanoyloxy]propyl]O1-[(Z)-dec-4-enyl]hexanedioate (0.1 g, 79.47 mol, 12.39% yield, 99% purity) was obtained as a colorless oil. ¹H NMR (400 MHz, CDCl₃) δ ppm 5.43-5.32 (m, 6H), 4.74 (br s, 1H), 4.19-3.99 (m, 15H), 3.45 (br s, 2H), 2.91 (br d, J=1.8 Hz, 4H), 2.39-2.31 (m, 14H), 2.10 (q, J=7.4 Hz, 6H), 2.05-1.82 (m, 12H), 1.73-1.62 (m, 20H), 1.42-1.19 (m, 28H), 0.98-0.78 (m, 12H).

### Example 55—Compound 48

### O6-[2,2-bis[[6-[(Z)-oct-3-enoxy]-6-oxo-hexanoyl]oxymethyl]-3-[4-(2-pyrrolidin-1ylethylcarbamoyloxy)decanoyloxy]propyl]O1-[(Z)-oct-3-enyl]hexanedioate

### Step 1:

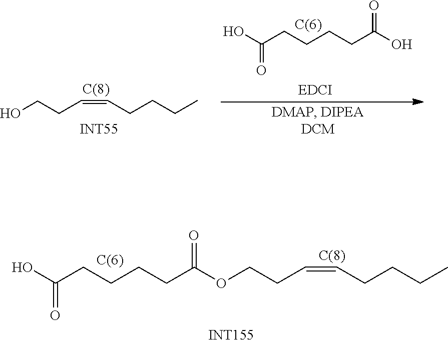

To a solution of (Z)-oct-3-en-1-ol (4 g, 31.20 mmol, 1 eq) in DCM (40 mL) was added DIPEA (12.10 g, 93.60 mmol, 16.30 mL, 3 eq), EDCI (7.78 g, 40.56 mmol, 1.3 eq) and DMAP (762.28 mg, 6.24 mmol, 0.2 eq) under N₂ atmosphere. Then adipic acid (22.80 g, 155.99 mmol, 25.91 mL, 5 eq) in DCM (220 mL) was added to the above reaction mixture. The reaction mixture was stirred at 25° C. for 12 hr under N₂ atmosphere. The reaction mixture was concentrated under reduced pressure to remove solvent. The residue was diluted with H₂O (1500 mL) and extracted with DCM 4500 mL (1500 mL*3). The combined organic layers were washed with aq. NaCl (1500 mL), dried over Na₂SO₄, filtered and the filtrate was concentrated under reduced pressure to give a residue. The residue was purified by column chromatography (SiO₂, Petroleum ether/Ethyl acetate=10/1). Compound 6-[(Z)-oct-3-enoxy]-6-oxo-hexanoic acid (25 g, 97.53 mmol, 62.52% yield) was obtained as a colorless oil.

### Step 2:

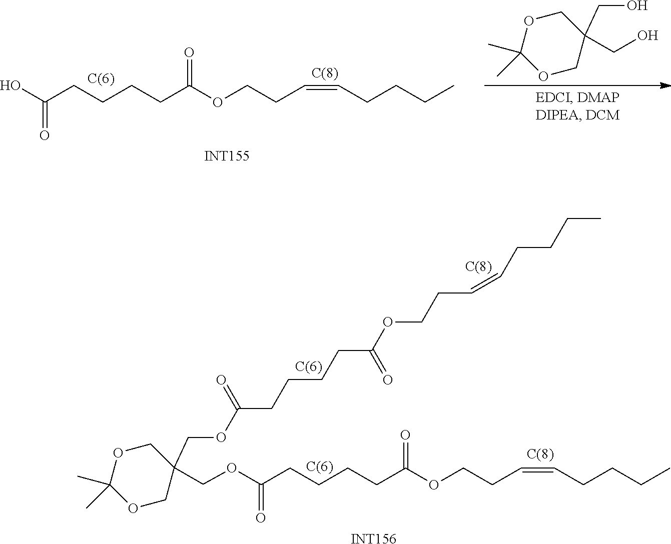

To a solution of 6-[(Z)-oct-3-enoxy]-6-oxo-hexanoic acid (3.23 g, 12.61 mmol, 1 PG eq) in DCM (32 mL) was added DIPEA (1.63 g, 12.61 mmol, 2.20 mL, 1 eq), EDCI (2.42 g, 12.61 mmol, 1 eq) and DMAP (1.54 g, 12.61 mmol, 1 eq) under N₂ atmosphere. Then [5-(hydroxymethyl)-2,2-dimethyl-1,3-dioxan-5-yl]methanol (1 g, 5.68 mmol, 0.45 eq) in DCM (10 mL) was added to the above reaction mixture. The reaction mixture was stirred at 25° C. for 12 hr under N₂ atmosphere. The reaction mixture was diluted with H₂O (150 mL) and extracted with DCM 450 mL (150 mL*3). The combined organic layers were washed with aq. NaCl (150 mL), dried over Na₂SO₄, filtered and the filtrate was concentrated under reduced pressure to give a residue. The residue was purified by column chromatography (SiO₂, Petroleum ether/Ethyl acetate=10/1 to 5/1). Compound O6-[[2,2-dimethyl-5-[[6-[(Z)-oct-3-enoxy]-6-oxo-hexanoyl]oxymethyl]-1,3-dioxan-5-yl]methyl]O1-[(Z)-oct-3-enyl]hexanedioate (1.5 g, 2.30 mmol, 18.22% yield) was obtained as a colorless oil. ¹H NMR (400 MHz, CDCl₃) δ ppm 5.52-5.49 (m, 2H), 5.35-5.33 (m, 2H), 4.11 (s, 2H), 4.07 (t, J=7.0 Hz, 4H), 3.74 (s, 2H), 2.46-2.25 (m, 12H), 2.05 (q, J=6.6 Hz, 4H), 1.77-1.53 (m, 12H), 1.42 (s, 6H), 1.38-1.25 (m, 8H), 0.98-0.80 (m, 6H).

### Step 3:

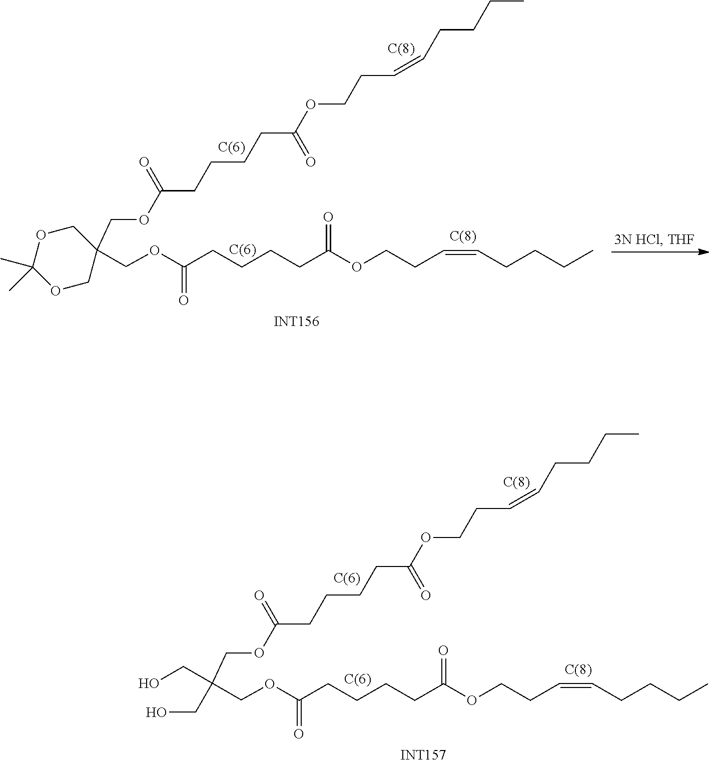 [SMILES: CCCC/C=C\CCOC(=O)CCCCC(=O)OCC1(COC(=O)CCCCC(=O)OCC/C=C\CCCC)COC(C)(C)OC1 ; "INT156" ; CCCC/C=C\CCOC(=O)CCCCC(=O)OCC(CO)(CO)COC(=O)CCCCC(=O)OCC/C=C\CCCC ; "INT157"]

To a solution of O6-[[2,2-dimethyl-5-[[6-[(Z)-oct-3-enoxy]-6-oxo-hexanoyl]oxymethyl]-1,3-dioxan-5-yl]methyl]O1-[(Z)-oct-3-enyl]hexanedioate (1.5 g, 2.30 mmol, 1 eq) in THF (82 mL) was added HCl (3 M, 842.46 L, 1.1 eq) at 0° C. under N₂ atmosphere. The reaction mixture was stirred at 25° C. for 12 hr under N₂ atmosphere. The reaction mixture was diluted with sat. NaHCO₃ (100 mL) and extracted with EtOAc 300 mL (100 mL*3). The combined organic layers were concentrated under reduced pressure to give a residue. The residue was purified by column chromatography (SiO₂, Petroleum ether/Ethyl acetate=1/1). Compound O6-[2,2-bis(hydroxymethyl)-3-[6-[(Z)-oct-3-enoxy]-6-oxo-hexanoyl]oxy-propyl]O1-[(Z)-oct-3-enyl]hexaneioate (800 mg, 1.31 mmol, 56.82% yield) was obtained as a colorless oil.

### Step 4:

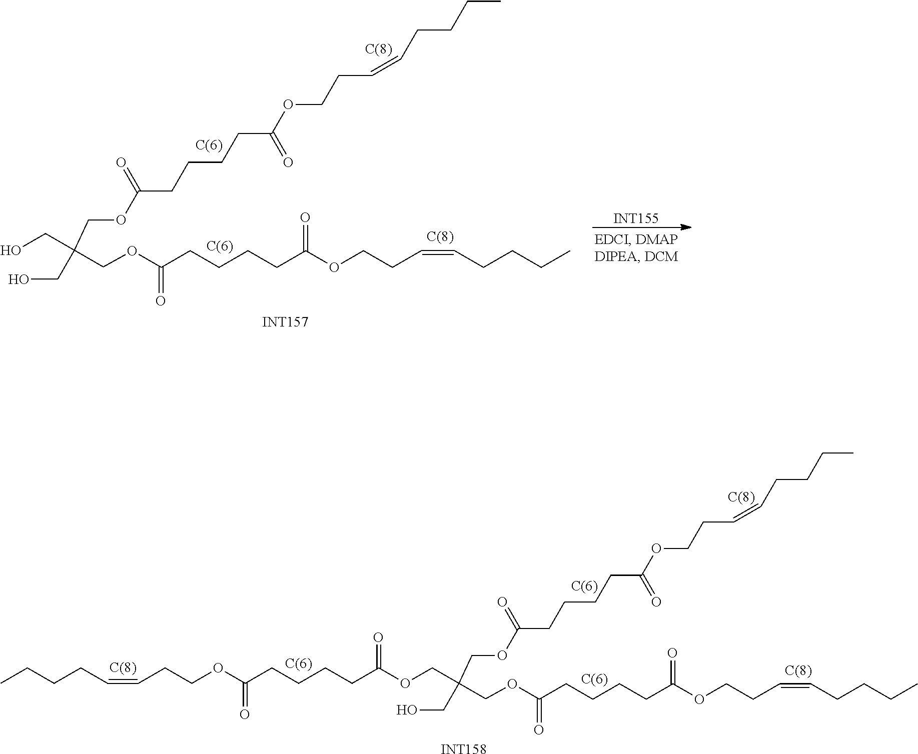

To a solution of O6-[2,2-bis(hydroxymethyl)-3-[6-[(Z)-oct-3-enoxy]-6-oxo-hexanoyl]oxy-propyl]O1-[(Z)-oct-3-enyl]hexanedioate (800 mg, 1.31 mmol, 1 eq) in DCM (24 mL) was added DIPEA (421.82 mg, 3.26 mmol, 568.49 L, 2.5 eq), EDCI (300.32 mg, 1.57 mmol, 1.2 eq) and DMAP (15.95 mg, 130.55 mol, 0.1 eq) under N₂ atmosphere. Then 6-[(Z)-oct-3-enoxy]-6-oxo-hexanoic acid (200.79 mg, 783.30 mol, 0.6 eq) in DCM (12 mL) was added to the above reaction mixture. The reaction mixture was stirred at 25° C. for 12 hr under N₂ atmosphere. The reaction mixture was concentrated under reduced pressure to remove solvent. The residue was diluted with H₂O (150 mL) and extracted with DCM 450 mL (150 mL*3). The combined organic layers were washed with aq. NaCl (150 mL), dried over Na₂SO₄, filtered and the filtrate was concentrated under reduced pressure to give a residue. The residue was purified by column chromatography (SiO₂, Petroleum ether/Ethyl acetate=2/1 to 1/1). Compound O6-[2-(hydroxymethyl)-3-[6-[(Z)-oct-3-enoxy]-6-oxo-hexanoyl]oxy-2-[[6-[(Z)-oct-3-enoxy]-6-oxohexanoyl]oxymethyl]propyl]O1-[(Z)-oct-3-enyl]hexanedioate (400 mg, 469.97 mol, 36.00% yield) was obtained as a colorless oil. ¹H NMR (400 MHz, CDCl₃) δ ppm 5.52-5.50 (m, 3H), 5.35-5.33 (m, 3H), 4.11-4.05 (m, 12 H), 3.52 (s, 2H), 2.41-2.31 (m, 18H), 2.09-2.01 (m, 6H), 1.66 (m, 12H), 1.38-1.28 (m, 12H), 0.95-0.85 (m, 9H).

### Step 5:

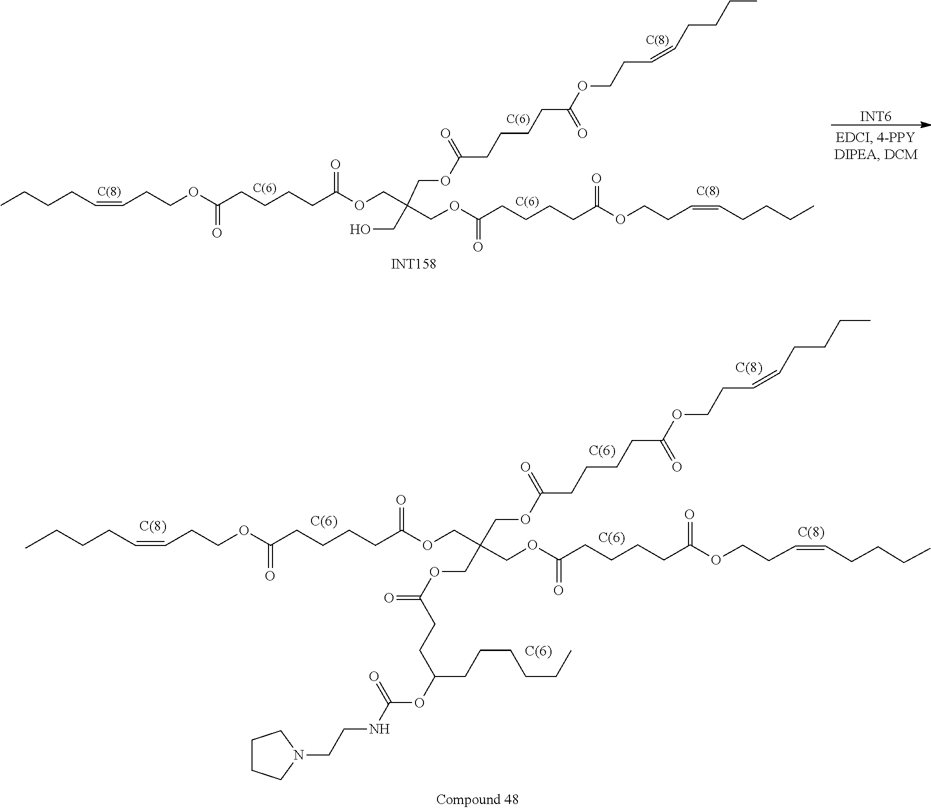

To a solution of O6-[2-(hydroxymethyl)-3-[6-[(Z)-oct-3-enoxy]-6-oxo-hexanoyl]oxy-2-[[6-[(Z)-oct-3-enoxy]-6-oxohexanoyl]oxymethyl]propyl]O1-[(Z)-oct-3-enyl]hexanedioate (200 mg, 234.99 mol, 1 eq) in DCM (7 mL) was added DIPEA (75.93 mg, 587.47 mol, 102.33 μL, 2.5 eq), EDCI (54.06 mg, 281.98 mol, 1.2 eq) and 4-pyrrolidin-1-ylpyridine (3.48 mg, 23.50 mol, 0.1 eq) under N₂ atmosphere. Then 1-(3-oxopropyl)heptyl N-(2-pyrrolidin-1-ylethyl)carbamate (88.10 mg, 281.98 mol, 1.2 eq) in DCM (3 mL) was added to the above reaction mixture. The reaction mixture was stirred at 25° C. for 12 hr under N₂ atmosphere. The reaction mixture was concentrated under reduced pressure to remove solvent to get a residue. The residue was diluted with H₂O (15 mL) and extracted with DCM 45 mL (15 mL*3). The combined organic layers were washed with aq. NaCl (150 mL), dried over Na₂SO₄, filtered and the filtrate was concentrated under reduced pressure to give a residue. The residue was purified by column chromatography (SiO₂, Petroleum ether/Ethyl acetate=2/1 to 1/1). Compound O6-[2,2-bis[[6-[(Z)-oct-3-enoxy]-6-oxo-hexanoyl]oxymethyl]-3-[4-(2-pyrrolidin-1ylethylcarbamoyloxy)decanoyloxy]propyl]O1-[(Z)-oct-3-enyl]hexanedioate (120 mg, 103.31 mol, 43.96% yield) was obtained as a colorless oil. ¹H NMR (400 MHz, CDCl₃) δ ppm 5.52-5.47 (m, 3H), 5.37-5.32 (m, 4H), 4.75 (s, 1H), 4.13-4.04 (m, 14H), 3.32 (d, J=4.8 Hz, 2H), 2.72-2.51 (m, 6H), 2.41-2.32 (m, 18H), 2.05 (q, J=6.6 Hz, 6H), 1.94-1.72 (m, 8H), 1.67-1.62 (m, 12H), 1.56-1.46 (m, 2H), 1.37-1.25 (m, 20H), 0.98-0.83 (m, 12H).

### Example 56—Compound 49

### O5-[2,2-bis[[5-[(Z)-non-3-enoxy]-5-oxo-pentanoyl]oxymethyl]-3-[4-(2-pyrrolidin-1-ylethylcarbamoyloxy)decanoyloxy]-propyl]O1-[(Z)-non-3-enyl]pentanedioate

### Step 1:

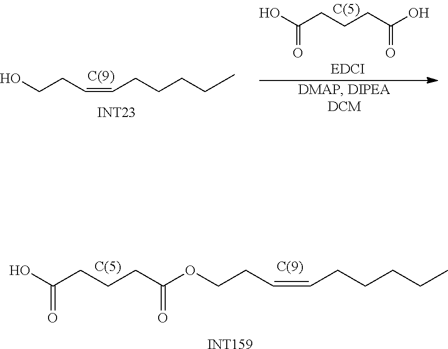

To a mixture of (Z)-non-3-en-1-ol (20 g, 140.61 mmol, 1 eq), DMAP (1.72 g, 14.06 mmol, 0.1 eq), DIPEA (45.43 g, 351.52 mmol, 61.23 mL, 2.5 eq) and EDCI (32.35 g, 168.73 mmol, 1.2 eq) in THF (600 mL) and DCM (600 mL) was added glutaric acid (92.88 g, 703.05 mmol, 5 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were washed with ethyl acetate 600 mL (200 mL*3), dried over Na₂SO₄, filtered and the filtrate was concentrated under reduced pressure to give a residue. The residue was purified by column chromatography (SiO₂, Petroleum ether/Ethyl acetate=100/1 to 0/1). Compound 5-[(Z)-non-3-enoxy]-5-oxo-pentanoic acid (25 g, 97.53 mmol, 69.36% yield) was obtained as a colorless oil. ¹H NMR (400 MHz, CDCl₃) δ ppm 5.58-5.45 (m, 1H), 5.40-5.23 (m, 1H), 4.09 (t, J=7.0 Hz, 2H), 2.51-2.33 (m, 6H), 2.11-1.90 (m, 4H), 1.42-1.20 (m, 6H), 0.90 (t, J=6.8 Hz, 3H).

### Step 2:

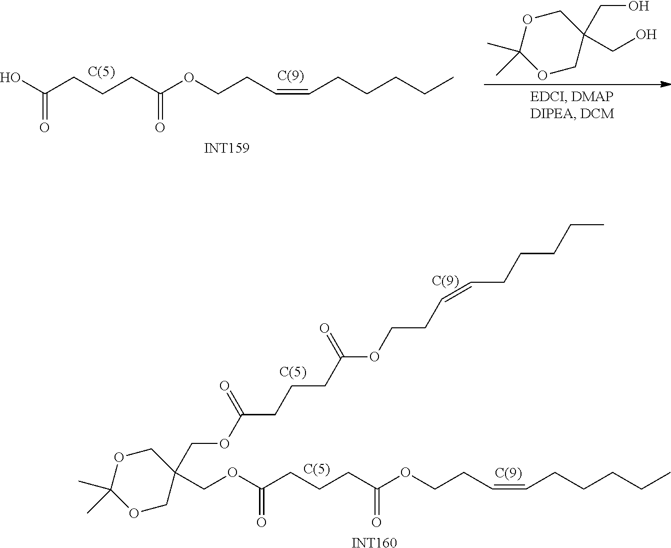

To a mixture of 5-[(Z)-non-3-enoxy]-5-oxo-pentanoic acid (3.23 g, 12.61 mmol, 1 eq), DIPEA (4.07 g, 31.53 mmol, 5.49 mL, 2.5 eq), DMAP (154.07 mg, 1.26 mmol, 0.1 eq) and EDCI (2.90 g, 15.13 mmol, 1.2 eq) in DCM (40 mL) was added [5-(hydroxymethyl)-2,2-dimethyl-1,3-dioxan-5-yl]methanol (1 g, 5.68 mmol, 0.45 eq) under N₂ atmosphere. The reaction mixture was stirred at 25° C. for 12 hr under N₂ atmosphere. The reaction mixture was diluted with H₂O (40 mL) and extracted with DCM 120 mL (40 mL*3). The combined organic layers were washed with ethyl acetate 120 mL (40 mL*3), dried over Na₂SO₄, filtered and the filtrate was concentrated under reduced pressure to give a residue. The residue was purified by column chromatography (SiO₂, Petroleum ether/Ethyl acetate=100/1 to 0/1). Compound O5-[[2,2-dimethyl-5-[[5-[(Z)-non-3-enoxy]-5-oxo-pentanoyl]oxymethyl]-1,3-dioxan-5-yl]methyl]O1-[(Z)-non-3-enyl]pentanedioate (1.5 g, 2.30 mmol, 18.22% yield) was obtained as a colorless oil. ¹H NMR (400 MHz, CDCl₃) δ ppm 5.57-5.44 (m, 2H), 5.39-5.26 (m, 2H), 4.12 (s, 4H), 4.07 (t, J=7.0 Hz, 4H), 3.74 (s, 4H), 2.44-2.32 (m, 12H), 2.04 (q, J=7.0 Hz, 4H), 1.99-1.90 (m, 4H), 1.42 (s, 6H), 1.39-1.24 (m, 12H), 0.89 (t, J=6.8 Hz, 6H).

### Step 3:

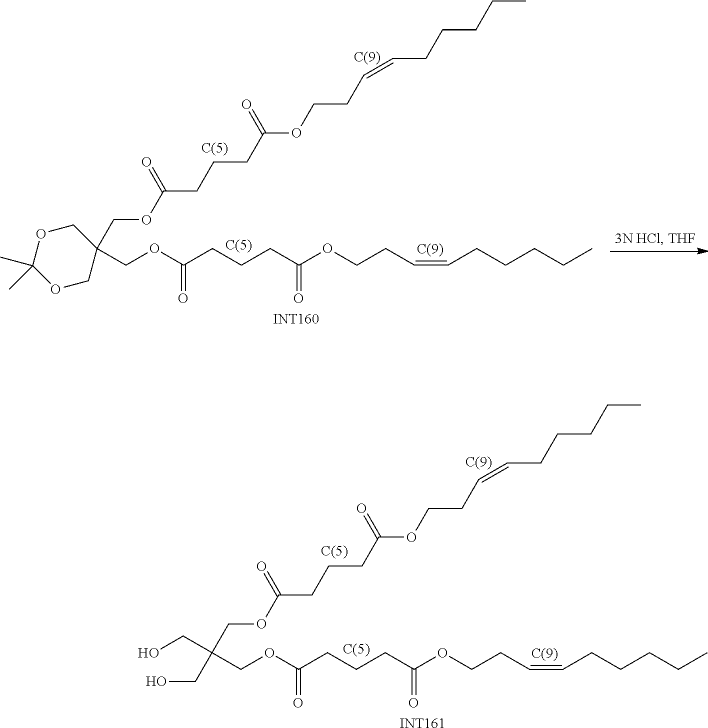

To a solution of 05-[[2,2-dimethyl-5-[[5-[(Z)-non-3-enoxy]-5-oxo-pentanoyl]oxymethyl]-1,3-dioxan-5-yl]methyl]O1-[(Z)-non-3-enyl]pentanedioate (5 g, 7.66 mmol, 1 eq) in THF (50 mL) was added HCl (3 M, 2.81 mL, 1.1 eq) at 0° C. under N₂ atmosphere. The reaction mixture was stirred at 25° C. for 12 hr under N₂ atmosphere. The reaction mixture was diluted with aq. NaHCO₃ (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O5-[2,2-bis(hydroxymethyl)-3-[5-[(Z)-non-3-enoxy]-5-oxo-pentanoyl]oxy-propyl]O1-[(Z)-non-3-enyl]pentanedioate (2 g, 3.26 mmol, 42.62% yield) was obtained as a colorless oil. H NMR (400 MHz, CDCl₃) δ ppm 5.58-5.47 (m, 2H) 5.39-5.26 (m, 2H), 4.14 (s, 4H), 4.08 (t, J=7.0 Hz, 4H), 3.59 (s, 4H), 2.45-2.36 (m, 12H), 2.06-1.93 (m, 8H), 1.37-1.23 (m, 12H), 0.89 (t, J=6.8 Hz, 6H).

### Step 4:

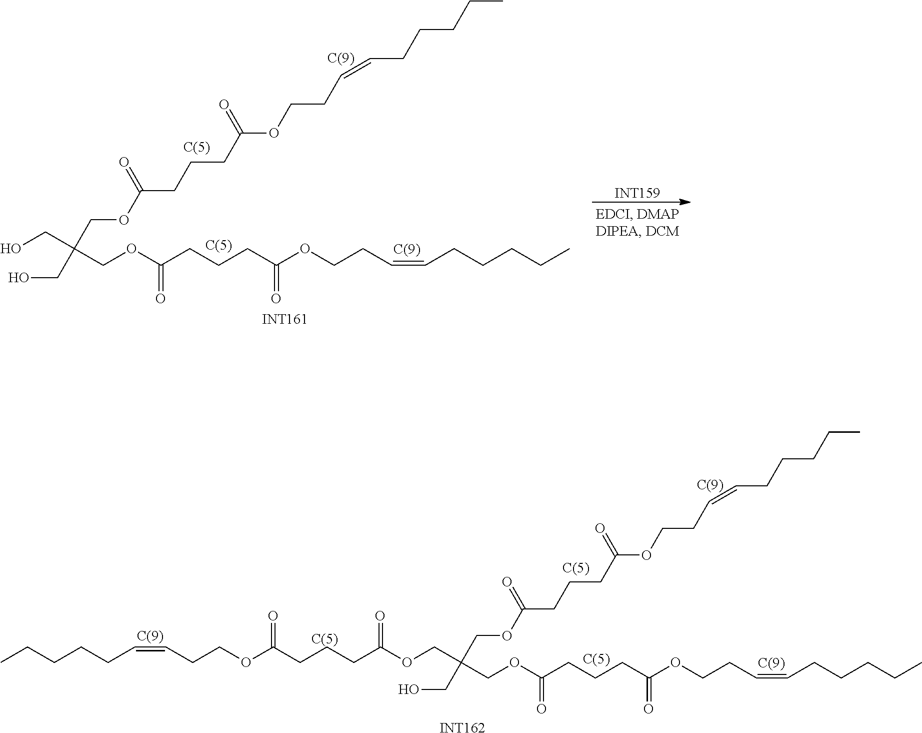

To a mixture of 05-[2,2-bis(hydroxymethyl)-3-[5-[(Z)-non-3-enoxy]-5-oxo-pentanoyl]oxy-propyl]O1-[(Z)-non-3-enyl]pentanedioate (2 g, 3.26 mmol, 1 eq), DMAP (39.87 mg, 326.38 mol, 0.1 eq), EDCI (750.80 mg, 3.92 mmol, 1.2 eq) and DIPEA (1.05 g, 8.16 mmol, 1.42 mL, 2.5 eq) in DCM (25 mL) was added 5-[(Z)-non-3-enoxy]-5-oxo-pentanoic acid (501.97 mg, 1.96 mmol, 0.6 eq) under N₂ atmosphere. The reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O5-[2-(hydroxymethyl)-3-[5-[(Z)-non-3-enoxy]-5-oxo-pentanoyl]oxy-2-[[5-[(Z)-non-3-enoxy]-5-oxopentanoyl]oxymethyl]propyl]O1-[(Z)-non-3-enyl]pentanedioate (0.8 g, 939.95 mol, 28.80% yield) was obtained as a colorless oil. ¹H NMR (400 MHz, CDCl₃) δ ppm 5.52-5.49 (m, 3H), 5.35-5.32 (m, 3H), 4.11-4.05 (m, 12H), 3.52 (s, 2H), 2.43-2.35 (m, 18H), 2.06-2.01 (m, 6H), 2.00-1.93 (m, 6H), 1.68-1.65 (m, 2H), 1.37-1.28 (m, 18H), 0.91-0.87 (m, 9H).

### Step 5:

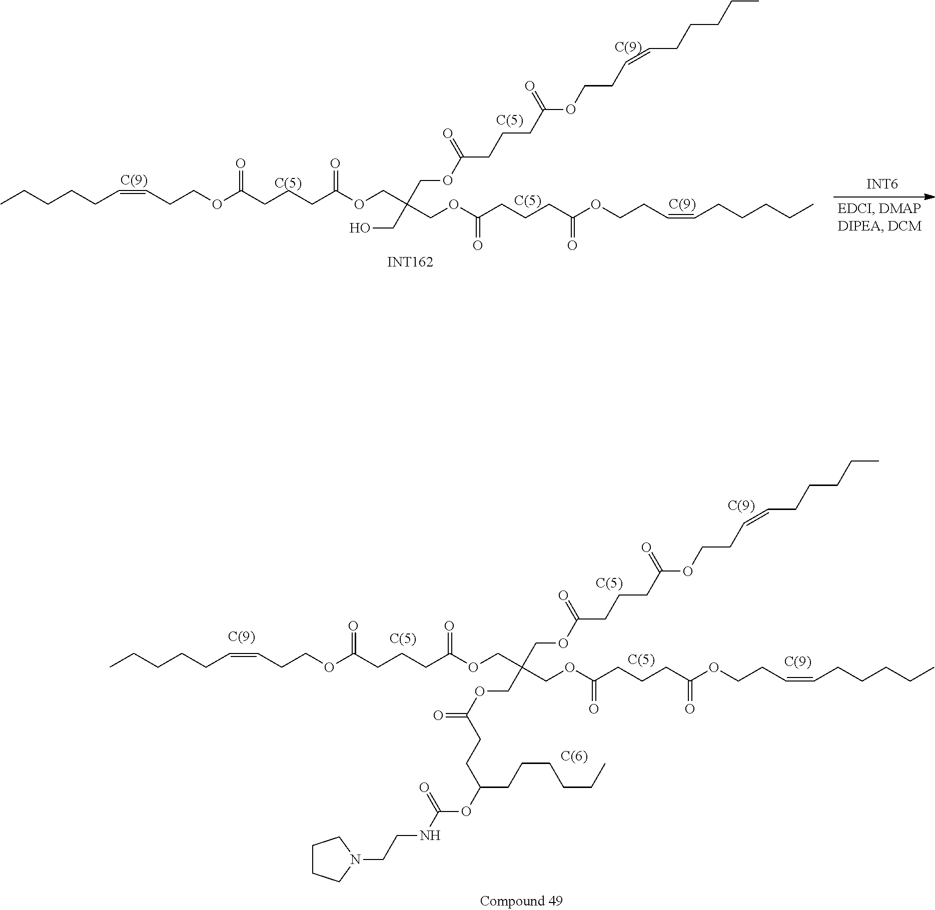

To a mixture of 05-[2-(hydroxymethyl)-3-[5-[(Z)-non-3-enoxy]-5-oxo-pentanoyl]oxy-2-[[5-[(Z)-non-3-enoxy]-5-oxo-pentanoyl]oxymethyl]propyl]O1-[(Z)-non-3-enyl]pentanedioate (0.8 g, 939.95 mol, 1 eq), DIPEA (121.48 mg, 939.95 mol, 163.72 L, 1 eq), DMAP (114.83 mg, 939.95 mol, 1 eq) and EDCI (180.19 mg, 939.95 mol, 1 eq) in DCM (12 mL) was added to 4-(2-pyrrolidin-1-ylethylcarbamoyloxy)decanoic acid (370.47 mg, 1.13 mmol, 1.2 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O5-[2,2-bis[[5-[(Z)-non-3-enoxy]-5-oxo-pentanoyl]oxymethyl]-3-[4-(2-pyrrolidin-1-ylethylcarbamoyloxy)decanoyloxy]-propyl]O1-[(Z)-non-3-enyl]pentanedioate (0.1 g, 86.09 mol, 9.16% yield, 100% purity) was obtained as a colorless oil. ¹H NMR (400 MHz, CDCl₃) δ ppm 5.52-5.48 (m, 3H), 5.35-5.32 (m, 3H), 4.75 (s, 1H), 4.16-4.06 (m, 14H), 3.44 (s, 2H) 2.89-2.76 (m, 4H), 2.42-2.35 (m, 20H), 2.07-2.00 (m, 6H), 1.97-1.90 (m, 10H), 1.83-1.72 (m, 2H), 1.60-1.48 (m, 12H), 1.38-1.26 (m, 26H), 0.91-0.86 (m, 12H).

### Example 57—Compound 50

### O6-[2,2-bis[(6-nonoxy-6-oxo-hexanoyl)oxymethyl]-3-[4-(2-pyrrolidin-1-ylethylcarbamoyloxy)decanoyloxy]propyl]O1-nonyl hexanedioate

### Synthetic Scheme:

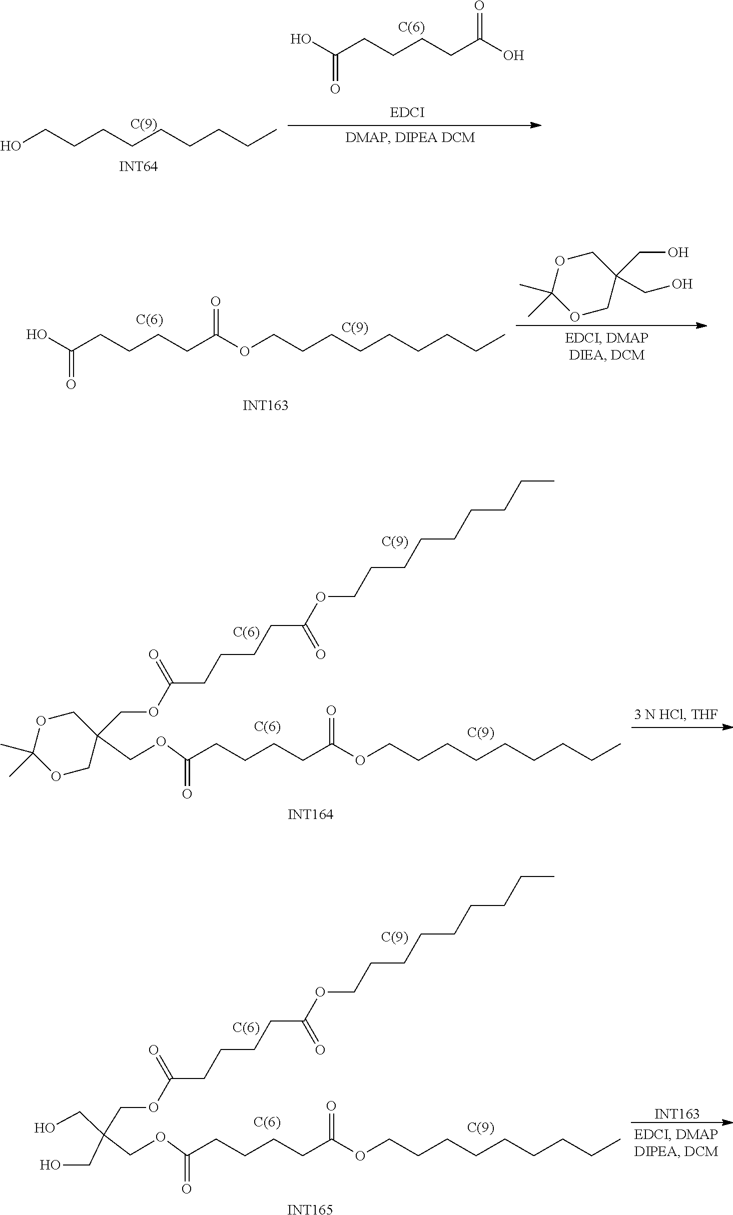

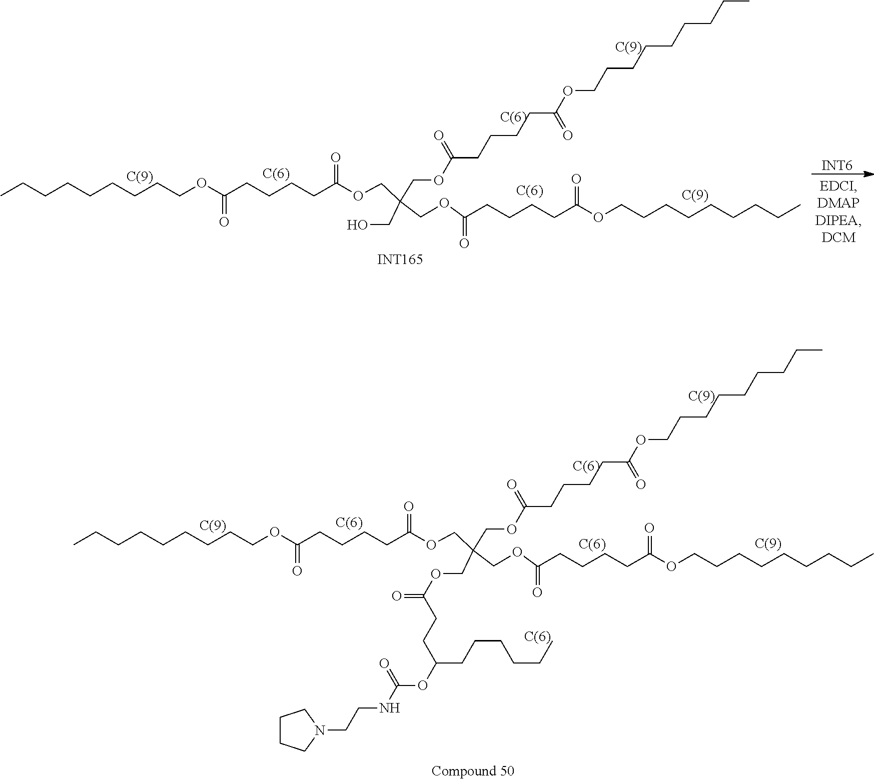

### Step 1:

To a mixture of adipic acid (20.26 g, 138.64 mmol, 23.02 mL, 5 eq), EDCI (6.38 g, 33.27 mmol, 1.2 eq), DIPEA (8.96 g, 69.32 mmol, 12.07 mL, 2.5 eq) and DMAP (338.76 mg, 2.77 mmol, 0.1 eq) in DCM (600 mL) and THF (600 mL) was added nonan-1-ol (4 g, 27.73 mmol, 1 eq) under N₂ atmosphere and the reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=10/1). Compound 6-nonoxy-6-oxo-hexanoic acid (23 g, 84.44 mmol, 60.90% yield) was obtained as a colorless oil.

### Step 2:

To a mixture of 6-nonoxy-6-oxo-hexanoic acid (10.31 g, 37.83 mmol, 1 eq), EDCI (8.70 g, 45.40 mmol, 1.2 eq), DIPEA (12.22 g, 94.58 mmol, 16.47 mL, 2.5 eq) and DMAP (462.20 mg, 3.78 mmol, 0.1 eq) in DCM (150 mL) was added [5-(hydroxymethyl)-2,2-dimethyl-1,3-dioxan-5-yl]methanol (3 g, 17.03 mmol, 0.45 eq) under N₂ atmosphere and the reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=5/1). Compound O6-[[2,2-dimethyl-5-[(6-nonoxy-6-oxo-hexanoyl)oxymethyl]-1,3-dioxan-5-yl]methyl]O1-nonyl hexanedioate (6 g, 8.76 mmol, 46.31% yield) was obtained as a colourless oil.

### Step 3:

To a solution of O6-[[2,2-dimethyl-5-[(6-nonoxy-6-oxo-hexanoyl)oxymethyl]-1,3-dioxan-5-yl]methyl]O1-nonyl hexanedioate (6 g, 8.76 mmol, 1 eq) in THF (100 mL) was added HCl (3 M, 3.21 mL, 1.1 eq) at 0° C. under N₂. The reaction mixture was stirred at 25° C. for 12 hr under N₂ atmosphere. The reaction mixture was diluted with H₂O (200 mL) and extracted with ethyl acetate 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/1). Compound O6-[2,2-bis(hydroxymethyl)-3-(6-nonoxy-6-oxo-hexanoyl)oxy-propyl]O1-nonyl hexanedioate (2 g, 3.10 mmol, 35.40% yield) was obtained as a colourless oil. ¹H NMR (400 MHz, CDCl₃) δ ppm 4.15-4.14 (m, 4H), 4.11-4.05 (m, 4H), 3.59 (s, 4H), 2.41-2.31 (m, 8H), 1.71-1.59 (m, 14H), 1.36-1.25 (m, 24H), 0.94-0.83 (m, 6H).

### Step 4:

To a solution of O6-[2,2-bis(hydroxymethyl)-3-(6-nonoxy-6-oxo-hexanoyl)oxy-propyl]O1-nonylhexanedioate (2 g, 3.10 mmol, 1 eq) in DCM (20 mL) was added EDCI (713.45 mg, 3.72 mmol, 1.2 eq), DMAP (37.89 mg, 310.14 mol, 0.1 eq) and DIPEA (1.00 g, 7.75 mmol, 1.35 mL, 2.5 eq) under N₂ atmosphere. Then 6-nonoxy-6-oxohexanoic acid (506.85 mg, 1.86 mmol, 0.6 eq) in DCM (20 mL) was added to the above reaction mixture. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=2/1 to 1/1). Compound O6-[2-(hydroxymethyl)-3-(6-nonoxy-6-oxo-hexanoyl)oxy-2-[(6-nonoxy-6-oxohexanoyl)oxymethyl]propyl]O1-nonyl hexanedioate (1 g, 1.11 mmol, 35.86% yield) was obtained as a colorless oil. ¹H NMR (400 MHz, CDCl₃) δ ppm 4.15-4.08 (m, 12H), 4.06-4.01 (m, 2H), 3.52 (s, 2H), 2.69 (s, 1H), 2.38-2.31 (m, 10H), 1.72-1.56 (m, 22H), 1.38-1.24 (m, 34H), 0.98-0.80 (m, 9H).

### Step 5:

To a solution of O6-[2-(hydroxymethyl)-3-(6-nonoxy-6-oxo-hexanoyl)oxy-2-[(6-nonoxy-6-oxohexanoy-1)oxymethyl]propyl]O1-nonyl hexanedioate (200 mg, 222.41 mol, 1 eq) in DCM (10 mL) was added DIPEA (71.86 mg, 556.03 mol, 96.85 μL, 2.5 eq), EDCI (51.16 mg, 266.89 mol, 1.2 eq), 4-pyrrolidin-1-ylpyridine (3.30 mg, 22.24 mol, 0.1 eq) under N₂ atmosphere. Then 4-(2-pyrrolidin-1-ylethylcarbamoyloxy)decanoic acid (73.05 mg, 222.41 mol, 1 eq) was added to the above reaction mixture. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=5/1 to 3/1). Compound O6-[2,2-bis[(6-nonoxy-6-oxo-hexanoyl)oxymethyl]-3-[4-(2-pyrrolidin-1-ylethylcarbamoyloxy)decanoyloxy]propyl]O1-nonyl hexanedioate (100 mg, 82.67 mol, 37.17% yield) was obtained as a pale yellow oil. ¹H NMR (400 MHz, CDCl₃) δ ppm 4.74 (s, 1H), 4.17-4.01 (m, 14H), 3.53 (d, J=17.0 Hz, 2H), 2.41-2.29 (m, 14H), 2.20-1.74 (m, 6H), 1.68-1.59 (m, 22H), 1.55-1.44 (m, 4H), 1.40-1.17 (m, 46H), 0.95-0.82 (m, 12H).

### Example 58—Compound 51

### O7-[2,2-bis[[7-(2-butyloctoxy)-7-oxo-heptanoyl]oxymethyl]-3-[4-(2-pyrrolidin-1ylethylcarbamoyloxy)decanoyloxy]propyl]O1-(2-butyloctyl) heptanedioate

### Synthetic Scheme:

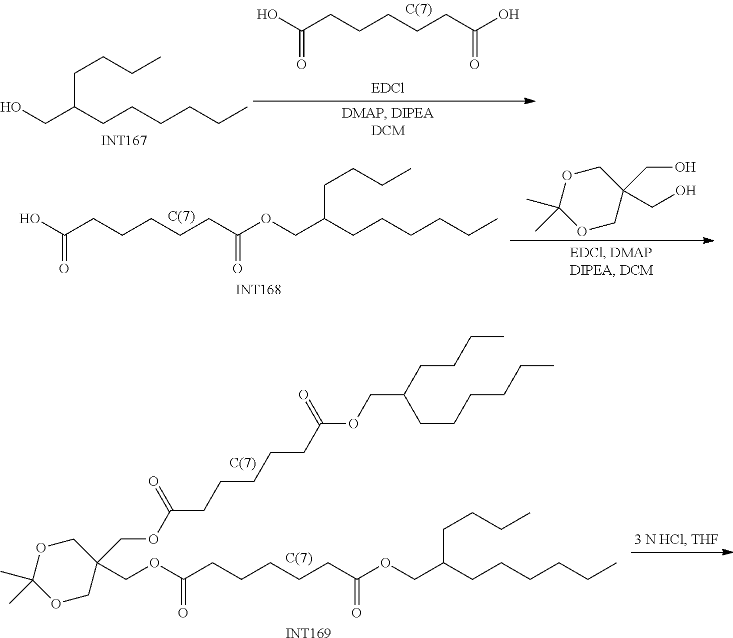

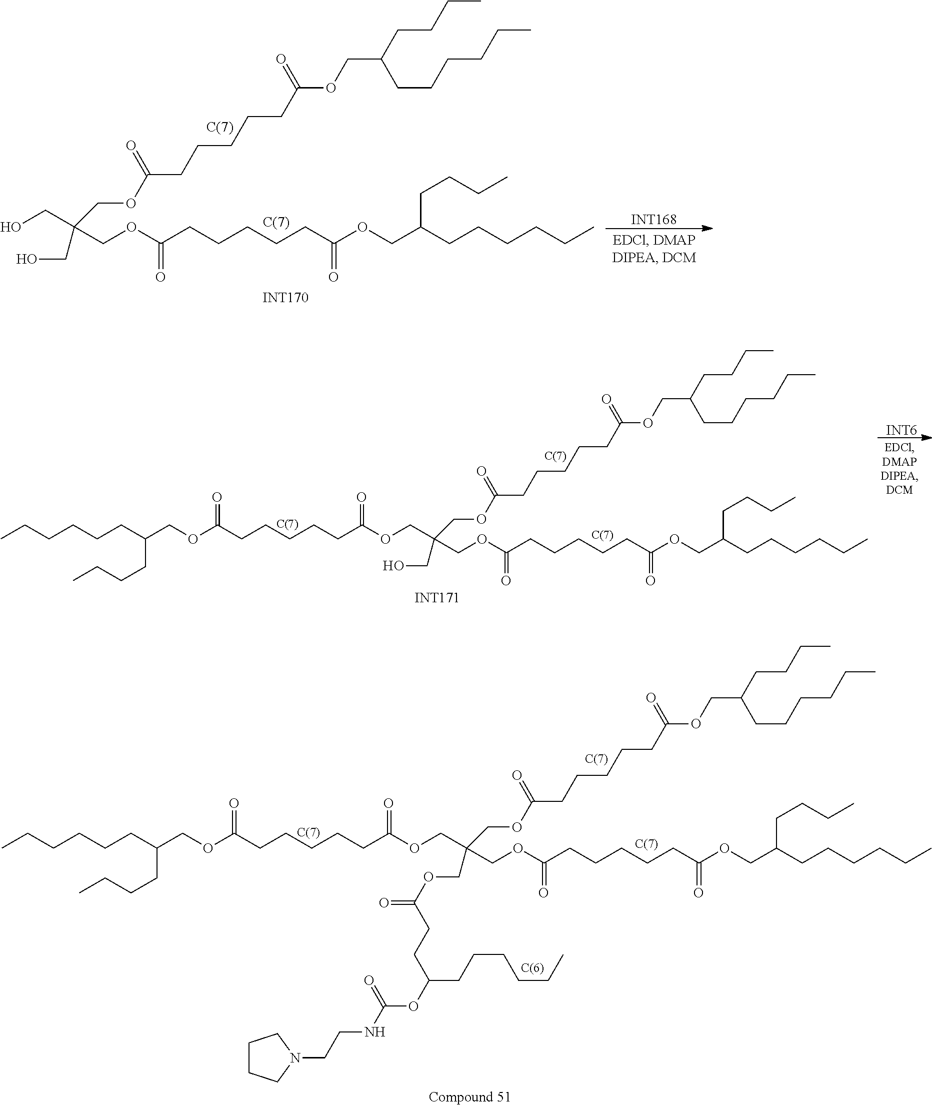

### Step 1:

To a solution of 2-butyloctan-1-ol (20 g, 107.33 mmol, 1 eq) in DCM (1000 mL) was added EDCI (24.69 g, 128.80 mmol, 1.2 eq), heptanedioic acid (85.96 g, 536.67 mmol, 5 eq), DMAP (1.31 g, 10.73 mmol, 0.1 eq) and DIPEA (34.68 g, 268.34 mmol, 46.74 mL, 2.5 eq) under N₂ atmosphere. The reaction mixture was stirred at 25° C. for 12 hr under N₂ atmosphere. The reaction mixture was diluted with H₂O (500 mL) and extracted with DCM 1500 mL (500 mL*3). The combined organic layers were dried over Na₂SO₄, filtered and the filtrate was concentrated under reduced pressure to give a residue. Compound 7-(2-butyloctoxy)-7-oxo-heptanoic acid (26 g, 79.15 mmol, 73.74% yield) was obtained as a colorless oil. ¹H NMR (400 MHz, CDCl₃) δ ppm 3.99-3.97, (d, J=2.8 Hz, 2H), 2.39-2.31 (m, 4H), 1.71-1.62 (m, 5H), 1.43-1.28 (m, 18H), 0.92-0.87 (m, 6H).

### Step 2:

To a solution of 7-(2-butyloctoxy)-7-oxo-heptanoic acid (12.43 g, 37.83 mmol, 1 eq) in DCM (150 mL) was added EDCI (8.70 g, 45.40 mmol, 1.2 eq), [5-(hydroxymethyl)-2,2-dimethyl-1,3-dioxan-5-yl]methanol (3 g, 17.03 mmol, 0.45 eq), DMAP (462.20 mg, 3.78 mmol, 0.1 eq) and DIPEA (12.22 g, 94.58 mmol, 16.47 mL, 2.5 eq) under N₂ atmosphere. The reaction mixture was stirred at 25° C. for 12 hr under N₂ atmosphere. The reaction mixture was diluted with H₂O (200 mL) and extracted with DCM 500 mL (100 mL*5). The combined organic layers were dried over Na₂SO₄, filtered and the filtrate was concentrated under reduced pressure to give a residue. Compound O7-[[5-[[7-(2-butyloctoxy)-7-oxo-heptanoyl]oxymethyl]-2,2-dimethyl-1,3-dioxan-5-yl]methyl]O1-(2-butyloctyl) heptanedioate (4.3 g, 5.39 mmol, 14.26% yield) was obtained as a colorless oil. ¹H NMR (400 MHz, CDCl₃) δ ppm 4.11 (s, 4H), 3.98 (d, J=3.0 Hz, 4H), 3.74 (s, 4H), 2.35-2.29 (m, 8H), 1.68-1.59 (m, 10H), 1.42-1.28 (m, 42H), 0.91-0.87 (m, 12H).

### Step 3:

To a solution of O7-[[5-[[7-(2-butyloctoxy)-7-oxo-heptanoyl]oxymethyl]-2,2-dimethyl-1,3-dioxan-5-yl]methyl]O1-(2-butyloctyl) heptanedioate (4.3 g, 5.39 mmol, 1 eq) in THF (54 mL) was added HCl (3 M, 1.98 mL, 1.1 eq) at 0° C. under N₂ atmosphere. The reaction mixture was stirred at 25° C. for 12 hr under N₂ atmosphere. The reaction mixture was diluted with aq. NaHCO₃ (100 mL) and extracted with EtOAc 400 mL (100 mL*4). The combined organic layers were dried over Na₂SO₄, filtered and the filtrate was concentrated under reduced pressure to give a residue. Compound O7-[2-[[7-(2-butyloctoxy)-7-oxo-heptanoyl]oxymethyl]-3-hydroxy-2-(hydroxymethyl)propyl]O1-(2-butyloctyl) heptanedioate (1.6 g, 2.11 mmol, 39.18% yield) was obtained as a colorless oil. ¹H NMR (400 MHz, CDCl₃) δ ppm 4.15 (s, 4H), 3.97 (d, J=5.8 Hz, 4H), 3.59 (s, 4H), 2.35 (m, 8H), 1.68-1.63 (m, 12H), 1.46-1.24 (m, 36H), 0.99-0.83 (m, 12H).

### Step 4:

To a solution of O7-[2-[[7-(2-butyloctoxy)-7-oxo-heptanoyl]oxymethyl]-3-hydroxy-2-(hydroxymethyl)propyl]O1-(2-butyloctyl) heptanedioate (1.6 g, 2.11 mmol, 1 eq) in DCM (20 mL) was added EDCI (486.16 mg, 2.54 mmol, 1.2 eq), 7-(2-butyloctoxy)-7-oxo-heptanoic acid (416.53 mg, 1.27 mmol, 0.6 eq), DMAP (25.82 mg, 211.34 mol, 0.1 eq) and DIPEA (682.84 mg, 5.28 mmol, 920.28 L, 2.5 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. Compound O7-[2,2-bis[[7-(2-butyloctoxy)-7-oxo-heptanoyl]oxymethyl]-3-hydroxy-propyl]O1-(2-butyloctyl)heptanedioate (0.9 g, 843.05 mol, 39.89% yield) was obtained as a colorless oil. ¹H NMR (400 MHz, CDCl₃) δ ppm 4.11 (s, 6H), 3.97 (d, J=3.0 Hz, 6H), 3.51 (s, 2H), 2.65-2.62 (m, 1H), 2.36-2.23 (m, 12H), 1.69-1.61 (m, 15 Hz), 1.40-1.28 (m, 54 Hz), 0.92-0.87 (m, 18H).

### Step 5:

To a solution of O7-[2,2-bis[[7-(2-butyloctoxy)-7-oxo-heptanoyl]oxymethyl]-3-hydroxy-propyl]O1-(2-butyloctyl) heptanedioate (0.9 g, 843.05 mol, 1 eq) in DCM (12 mL) was added EDCI (193.94 mg, 1.01 mmol, 1.2 eq), 4-(2-pyrrolidin-1-ylethylcarbamoyloxy)decanoic acid (166.14 mg, 505.83 mol, 0.6 eq), DMAP (10.30 mg, 84.30 mol, 0.1 eq) and DIPEA (272.39 mg, 2.11 mmol, 367.11 L, 2.5 eq) under N₂ atmosphere. The reaction mixture was stirred at 25° C. for 12 hr under N₂ atmosphere. The reaction mixture was diluted with H₂O (50 mL) and extracted with DCM 100 mL (25 mL*4). The combined organic layers were dried over Na₂SO₄, filtered and the filtrate was concentrated under reduced pressure to give a residue. Compound O7-[2,2-bis[[7-(2-butyloctoxy)-7-oxo-heptanoyl]oxymethyl]-3-[4-(2-pyrrolidin-1ylethylcarbamoyloxy)decanoyloxy]propyl]O1-(2-butyloctyl) heptanedioate (0.13 g, 94.34 mol, 11.19% yield) was obtained as a colorless oil. ¹H NMR (400 MHz, CDCl₃) δ ppm 5.32-5.28 (s, 1H), 4.75 (s, 1H), 4.11 (s, 8H), 3.98 (d, J=2.8 Hz, 6H), 3.33-3.32 (s, 2H), 2.62 (d, J=4.8 Hz, 6H), 2.40-2.29 (m, 14H), 1.81 (s, 6H), 1.68-1.59 (m, 16H), 1.39-1.28 (m, 64H), 0.91-0.86 (m, 21H).

### Example 59—Compound 52

### O7-[2-(hydroxymethyl)-3-[7-oxo-7-(3-pentyloctoxy)heptanoyl]oxy-2-[[7-oxo-7-(3-pentyloctoxy)heptanoyl]oxy-methyl]propyl]O1-(3-pentyloctyl) heptanedioate

### Synthetic Scheme:

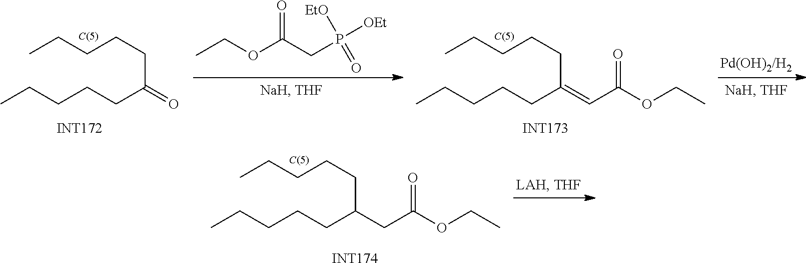

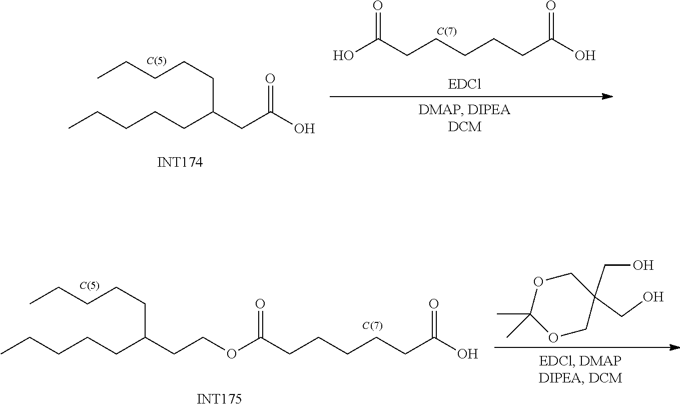

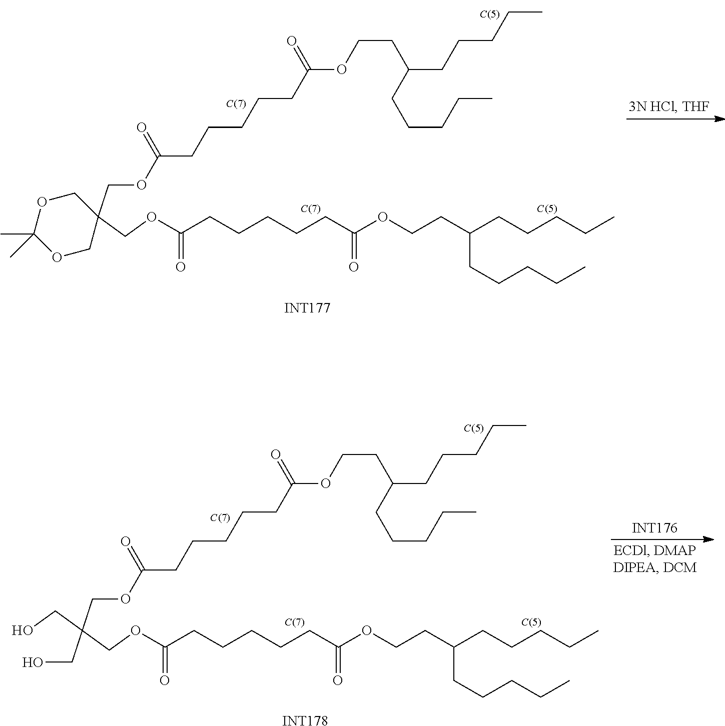

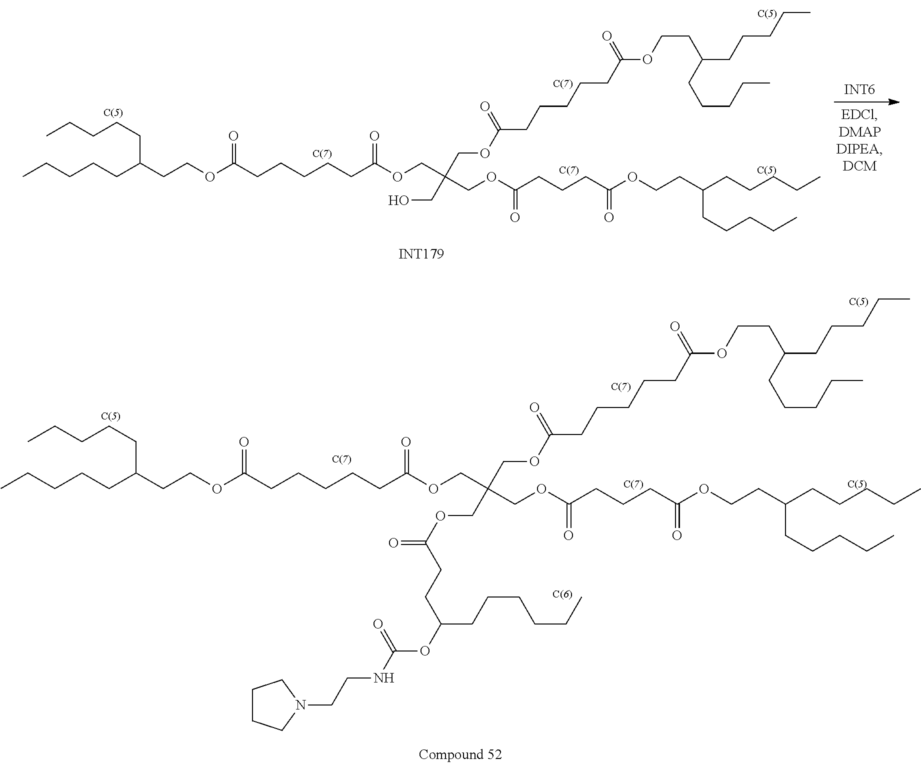

### Step 1:

To a solution of undecan-6-one (10 g, 58.72 mmol, 1 eq) in THF (100 mL) was added NaH (3.05 g, 76.34 mmol, 60% purity, 1.3 eq) portionwise at 0° C. under N₂ atmosphere. The reaction mixture was stirred at 25° C. for 1 h under N₂ atmosphere. Then ethyl 2-diethoxyphosphorylacetate (17.11 g, 76.34 mmol, 15.15 mL, 1.3 eq) in THF (100 mL) was added to the above solution dropwise at 25° C. under N₂ atmosphere and the reaction mixture was stirred for another 11 h at 25° C. under N₂ atmosphere. The reaction mixture was added sat. NH₄Cl (200 mL) slowly under N₂ atmosphere and stirred for another 10 min after addition. The reaction mixture was poured into water (200 mL) and extracted with EtOAc 600 ml (3*200 ml). The organic phase was evaporated under reduced pressure to get a residue. Compound ethyl 3-pentyloct-2-enoate (10 g, 41.60 mmol, 70.84% yield) was obtained as a colorless oil. ¹H NMR (400 MHz, CDCl₃) δ ppm 5.62 (s, 1H), 4.14 (q, J=7.2 Hz, 2H), 2.51-2.65 (m, 2H), 2.09-2.20 (m, 2H), 1.26-1.48 (m, 15H), 0.90 (t, J=6.4 Hz, 6H).

### Step 2:

To a solution of ethyl 3-pentyloct-2-enoate (10 g, 41.60 mmol, 1 eq) in THF (100 mL) was added Pd/C (44.27 g, 41.60 mmol, 10% purity, 1 eq) under N₂ atmosphere. The suspension was degassed and purged with H₂ for 3 times. The reaction mixture was stirred under H₂ (15 Psi) at 25° C. for 12 hr. The reaction mixture was filtered and the filtrate was concentrated under reduced pressure to give a residue. The residue was purified by column chromatography (SiO₂, Petroleum ether/Ethyl acetate=100/1 to 10/1). Compound ethyl 3-pentyloctanoate (8.5 g, 35.07 mmol, 84.29% yield) was obtained as a colorless oil. ¹H NMR (400 MHz, CDCl₃) δ ppm 4.16-4.10 (m, 2H), 2.23-2.22 (m, 2H), 1.86-1.84 (m, 1H), 1.31-1.24 (m, 19H), 0.89 (t, J=3.4 Hz, 6H).

### Step 3:

To a solution of LiAlH₄ (2.5 M, 28.05 mL, 2 eq) in THF (85 mL) was added ethyl 3-pentyloctanoate (8.5 g, 35.07 mmol, 1 eq) dropwise slowly at 0° C. under N₂ atmosphere. The reaction mixture was stirred for 12 h at 25° C. under N₂ atmosphere after addition. The reaction mixture was added sat. NH₄Cl (500 ml) slowly at 0° C. under N₂ atmosphere and stirred for another 10 min after addition. Then it was extracted with EtOAc and the organic phase was evaporated under reduced pressure to get a residue. The residue was purified by column chromatography (SiO₂, Petroleum ether/Ethyl acetate=100/1 to 0/1). Compound 3-pentyloctan-1-ol (5 g, 24.96 mmol, 71.17% yield) was obtained as a colorless oil. ¹H NMR (400 MHz, CDCl₃) δ ppm 3.68-3.64 (m, 2H), 1.55-1.42 (m, 4H), 1.31-1.26 (m, 16H), 0.89 (t, J=3.4 Hz, 6H).

### Step 4:

To a solution of 3-pentyloctan-1-ol (5 g, 24.96 mmol, 1 eq) in DCM (50 mL) was added EDCI (5.74 g, 29.95 mmol, 1.2 eq), heptanedioic acid (7.99 g, 49.91 mmol, 2 eq), DIPEA (8.06 g, 62.39 mmol, 10.87 mL, 2.5 eq) and DMAP (304.87 mg, 2.50 mmol, 0.1 eq) under N₂ atmosphere. The reaction mixture was stirred at 25° C. for 12 hr under N₂ atmosphere. The reaction mixture was concentrated under reduced pressure to remove solvent to get a residue. The residue was diluted with H₂O (200 mL) and extracted with DCM 600 mL (200 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound 7-oxo-7-(3-pentyloctoxy) heptanoic acid (5 g, 14.60 mmol, 58.50% yield) was obtained as a colorless oil.

### Step 5:

To a solution of 7-oxo-7-(3-pentyloctoxy) heptanoic acid (12.96 g, 37.83 mmol, 1 eq) in DCM (160 mL) was added EDCI (8.70 g, 45.40 mmol, 1.2 eq), [5-(hydroxymethyl)-2,2-dimethyl-1,3-dioxan-5-yl]methanol (3 g, 17.03 mmol, 0.45 eq), DIPEA (12.22 g, 94.58 mmol, 16.47 mL, 2.5 eq) and DMAP (462.20 mg, 3.78 mmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (200 mL) and extracted with DCM 600 mL (200 mL*3). The combined organic layers were filtered and the filtrate was concentrated under reduced pressure to give a residue. Compound O7-[[2-methyl-5-[[7-oxo-7-(3-pentyloctoxy) heptanoyl]oxymethyl]-1,3-dioxan-5-yl]methyl]O1-(3-pentyloctyl) heptanedioate (8.5 g, 10.48 mmol, 27.70% yield) was obtained as a colorless oil.

### Step 6:

To a solution of 7-oxo-7-(3-pentyloctoxy) heptanoic acid (12.96 g, 37.83 mmol, 1 eq) in DCM (160 mL) was added EDCI (8.70 g, 45.40 mmol, 1.2 eq), [5-(hydroxymethyl)-2,2-dimethyl-1,3-dioxan-5-yl]methanol (3 g, 17.03 mmol, 0.45 eq), DIPEA (12.22 g, 94.58 mmol, 16.47 mL, 2.5 eq) and DMAP (462.20 mg, 3.78 mmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (200 mL) and extracted with DCM 600 mL (200 mL*3). The combined organic layers were filtered and the filtrate was concentrated under reduced pressure to give a residue. Compound O7-[[2-methyl-5-[[7-oxo-7-(3-pentyloctoxy)heptanoyl]oxymethyl]-1,3-dioxan-5-yl]methyl]O1-(3-pentyloctyl) heptanedioate (8.5 g, 10.48 mmol, 27.70% yield) was obtained as a colorless oil.

### Step 7:

To a solution of O7-[2,2-bis(hydroxymethyl)-3-[7-oxo-7-(3-pentyloctoxy)heptanoyl]oxy-propyl]O1-(3-pentyloctyl) heptanedioate (0.8 g, 1.02 mmol, 1 eq) in DCM (10 mL) was added EDCI (234.40 mg, 1.22 mmol, 1.2 eq), 7-oxo-7-(3-pentyloctoxy)heptanoic acid (209.40 mg, 611.36 mol, 0.6 eq), DIPEA (329.22 mg, 2.55 mmol, 443.70 μL, 2.5 eq) and DMAP (12.45 mg, 101.89 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (20 mL) and extracted with DCM 60 mL (20 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O7-[2-(hydroxymethyl)-3-[7-oxo-7-(3-pentyloctoxy) heptanoyl]oxy-2-[[7-oxo-7-(3-pentyloctoxy)heptanoyl]-oxymethyl]propyl]O1-(3-pentyloctyl) heptanedioate (0.3 g, 270.36 mol, 26.53% yield) was obtained as a colorless oil. ¹H NMR (400 MHz, CDCl₃) δ ppm 4.11-4.06 (m, 12H), 3.51 (s, 2H), 2.65 (s, 1H), 2.29-2.21 (m, 12H), 1.59-1.49 (m, 20H), 1.30-1.18 (m, 58H), 0.82 (t, J=6.8 Hz, 18H).

### Step 8:

To a solution of O7-[2,2-bis(hydroxymethyl)-3-[7-oxo-7-(3-pentyloctoxy)heptanoyl]oxy-propyl]O1-(3-pentyloctyl) heptanedioate (0.8 g, 1.02 mmol, 1 eq) in DCM (10 mL) was added EDCI (234.40 mg, 1.22 mmol, 1.2 eq), 7-oxo-7-(3-pentyloctoxy)heptanoic acid (209.40 mg, 611.36 mol, 0.6 eq), DIPEA (329.22 mg, 2.55 mmol, 443.70 L, 2.5 eq) and DMAP (12.45 mg, 101.89 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (20 mL) and extracted with DCM 60 mL (20 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O7-[2-(hydroxymethyl)-3-[7-oxo-7-(3-pentyloctoxy) heptanoyl]oxy-2-[[7-oxo-7-(3-pentyloctoxy)heptanoyl]oxy-methyl]propyl]O1-(3-pentyloctyl) heptanedioate (0.3 g, 270.36 mol, 26.53% yield) was obtained as a colorless oil. ¹H NMR (400 MHz, CDCl₃) δ ppm 5.34-5.07 (m, 1H), 4.75 (s, 1H), 4.14-4.07 (m, 14H), 3.33 (s, 2H), 2.67-2.38 (m, 6H), 2.40-2.28 (m, 14H), 1.91-1.70 (m, 6H), 1.66-1.46 (m, 24H), 1.39-1.23 (m, 69H), 0.91-0.87 (m, 21H).

### Example 60—Compound 53

### O7-[[2-methyl-5-[[7-oxo-7-(3-pentyloctoxy) heptanoyl]oxymethyl]-1,3-dioxan-5-yl]methyl]O1-(3-pentyloctyl) heptanedioate

### Synthetic Scheme:

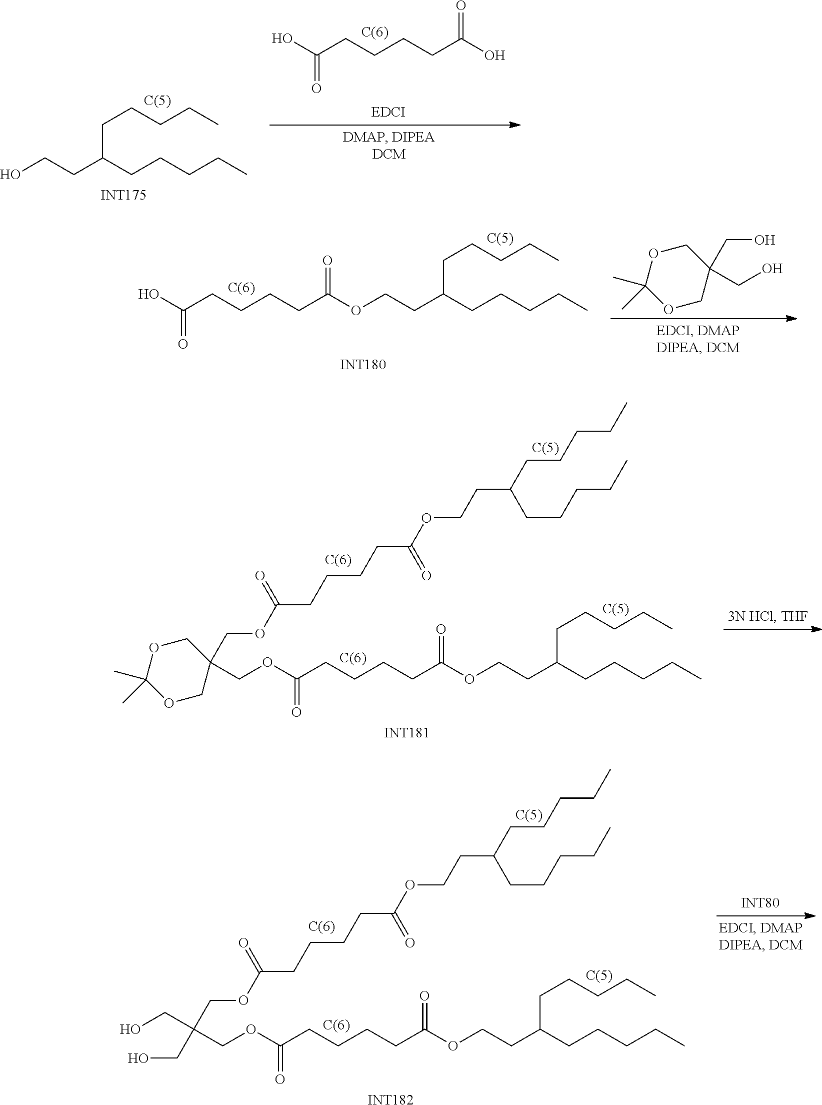

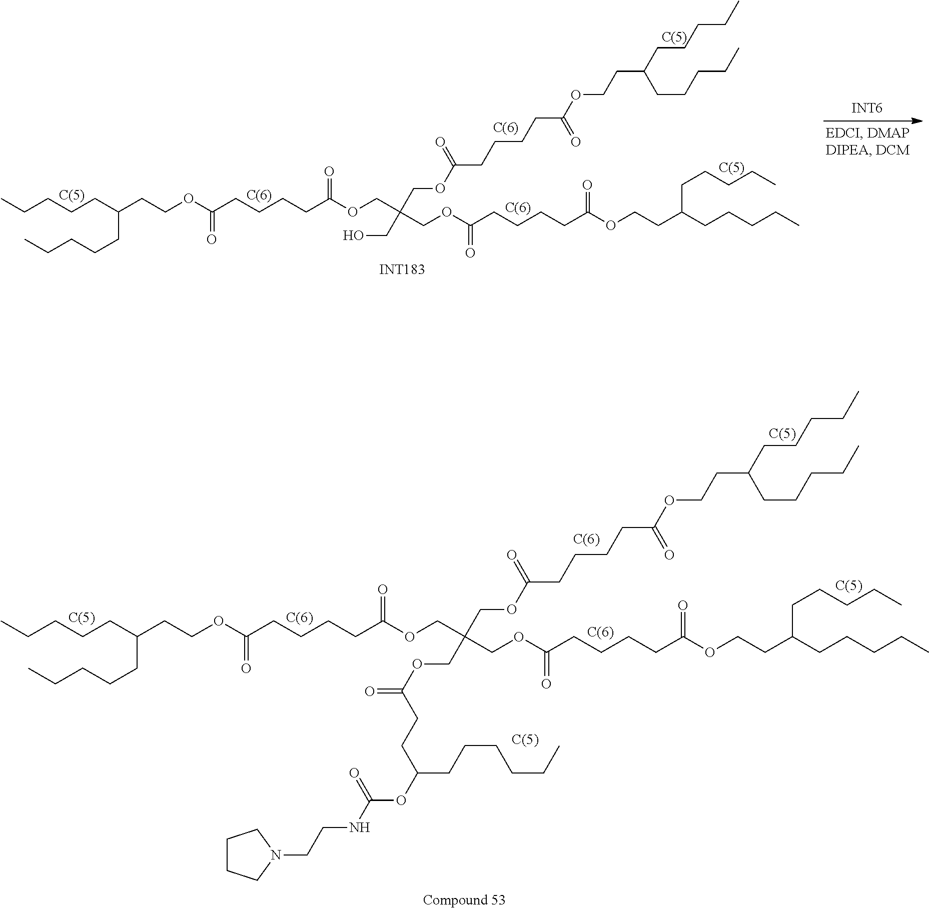

### Step 1:

To a solution of 3-pentyloctan-1-ol (3 g, 14.97 mmol, 1 eq) in THF (70 mL) and DCM (70 mL) was added EDCI (3.44 g, 17.97 mmol, 1.2 eq), adipic acid (10.94 g, 74.87 mmol, 12.43 mL, 5 eq), DIPEA (4.84 g, 37.43 mmol, 6.52 mL, 2.5 eq) and DMAP (182.92 mg, 1.50 mmol, 0.1 eq) under N₂ atmosphere. The reaction mixture was stirred at 25° C. for 12 hr under N₂ atmosphere. The reaction mixture was concentrated under reduced pressure to remove solvent to get a residue. The residue was diluted with H₂O (200 mL) and extracted with DCM 600 mL (200 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound 6-oxo-6-(3-pentyloctoxy) hexanoic acid (2.5 g, 7.61 mmol, 50.83% yield) was obtained as a colorless oil.

### Step 2:

To a solution of 6-oxo-6-(3-pentyloctoxy) hexanoic acid (2.5 g, 7.61 mmol, 1 eq) in DCM (35 mL) was added EDCI (1.75 g, 9.13 mmol, 1.2 eq), [5-(hydroxymethyl)-2,2-dimethyl-1,3-dioxan-5-yl]methanol (1.01 g, 5.71 mmol, 0.75 eq), DIPEA (2.46 g, 19.03 mmol, 3.31 mL, 2.5 eq) and DMAP (92.98 mg, 761.07 mol, 0.1 eq) under N₂ atmosphere. The reaction mixture was stirred at 25° C. for 12 hr under N₂ atmosphere. The reaction mixture was concentrated under reduced pressure to remove solvent to get a residue. The residue was diluted with H₂O (50 mL) and extracted with DCM 180 mL (60 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O6-[[2,2-dimethyl-5-[[6-oxo-6-(3-pentyloctoxy) hexanoyl]oxymethyl]-1,3-dioxan-5-yl]methyl]O1-(3-pentyloctyl) hexanedioate (2 g, 2.51 mmol, 32.97% yield) was obtained as a colorless oil.

### Step 3:

To a solution of O6-[[2,2-dimethyl-5-[[6-oxo-6-(3-pentyloctoxy) hexanoyl]oxymethyl]-1,3-dioxan-5-yl]methyl]O1-(3-pentyloctyl) hexanedioate (2 g, 2.51 mmol, 1 eq) in THF (20 mL) was added HCl (3 M, 919.94 L, 1.1 eq) at 0° C. under N₂ atmosphere. The reaction mixture was stirred at 25° C. for 12 hr under N₂ atmosphere. The reaction mixture was concentrated under reduced pressure to remove solvent. The residue was diluted with H₂O (40 mL) and extracted with EtOAc 120 mL (40 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O6-[2,2-bis(hydroxymethyl)-3-[6-oxo-6-(3-pentyloctoxy) hexanoyl]oxy-propyl]O1-(3-pentyloctyl) hexanedioate (0.8 g, 1.06 mmol, 42.12% yield) was obtained as a colorless oil.

### Step 4:

To a solution of O6-[2,2-bis(hydroxymethyl)-3-[6-oxo-6-(3-pentyloctoxy)hexanoyl]oxy-propyl]O1-(3-pentyloctyl) hexanedioate (0.8 g, 1.06 mmol, 1 eq) in DCM (10 mL) was added EDCI (243.08 mg, 1.27 mmol, 1.2 eq), 6-oxo-6-(3-pentyloctoxy)hexanoic acid (208.26 mg, 634.01 mol, 0.6 eq), DIPEA (341.42 mg, 2.64 mmol, 460.14 μL, 2.5 eq) and DMAP (12.91 mg, 105.67 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (50 mL) and extracted with DCM 180 mL (60 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-3-[6-oxo-6-(3-pentyloctoxy) hexanoyl]oxy-2-[[6-oxo-6-(3-pentyloctoxy)hexanoyl]oxymethyl]propyl]O1-(3-pentyloctyl) hexanedioate (0.3 g, 281.02 mol, 26.59% yield) was obtained as a colorless oil. ¹H NMR (400 MHz, CDCl₃) δ ppm 4.12-4.07 (m, 12H), 3.52 (s, 2H), 2.38-2.30 (m, 12H), 1.68-1.64 (m, 12H), 1.60-1.55 (m, 8H), 1.40 (s, 4H), 1.33-1.23 (m, 49H), 0.908-0.873 (t, J=3.4 Hz, 18H).

### Step 5:

To a solution of 7-oxo-7-(3-pentyloctoxy) heptanoic acid (12.96 g, 37.83 mmol, 1 eq) in DCM (160 mL) was added EDCI (8.70 g, 45.40 mmol, 1.2 eq), [5-(hydroxymethyl)-2,2-dimethyl-1,3-dioxan-5-yl]methanol (3 g, 17.03 mmol, 0.45 eq), DIPEA (12.22 g, 94.58 mmol, 16.47 mL, 2.5 eq) and DMAP (462.20 mg, 3.78 mmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (200 mL) and extracted with DCM 600 mL (200 mL*3). The combined organic layers were filtered and the filtrate was concentrated under reduced pressure to give a residue. Then it was purified by column. Compound O7-[[2-methyl-5-[[7-oxo-7-(3-pentyloctoxy) heptanoyl]oxymethyl]-1,3-dioxan-5-yl]methyl]O1-(3-pentyloctyl) heptanedioate (8.5 g, 10.48 mmol, 27.70% yield) was obtained as a colorless oil. ¹H NMR (400 MHz, CDCl₃) δ ppm 5.47-5.30 (m, 1H), 4.79 (d, J=14.8 Hz, 1H), 4.11-4.065 (m, 14H), 3.32-3.31 (m, 2H), 2.62 (d, J=11.8 Hz, 6H), 2.40-2.30 (m, 14H), 1.81 (m, 7H), 1.67-1.63 (m, 28H), 1.61-1.55 (m, 4H), 1.40-1.26 (m, 56H), 0.91-0.86 (m, 21H).

### Example 61—Compound 54

### O5-[2,2-bis[[5-oxo-5-(3-pentyloctoxy) pentanoyl]oxymethyl]-3-[4-(2-pyrrolidin-1-ylethylcarbamoyloxy)decanoyloxy]propyl]O1-(3-pentyloctyl) pentanedioate

### Synthetic Scheme:

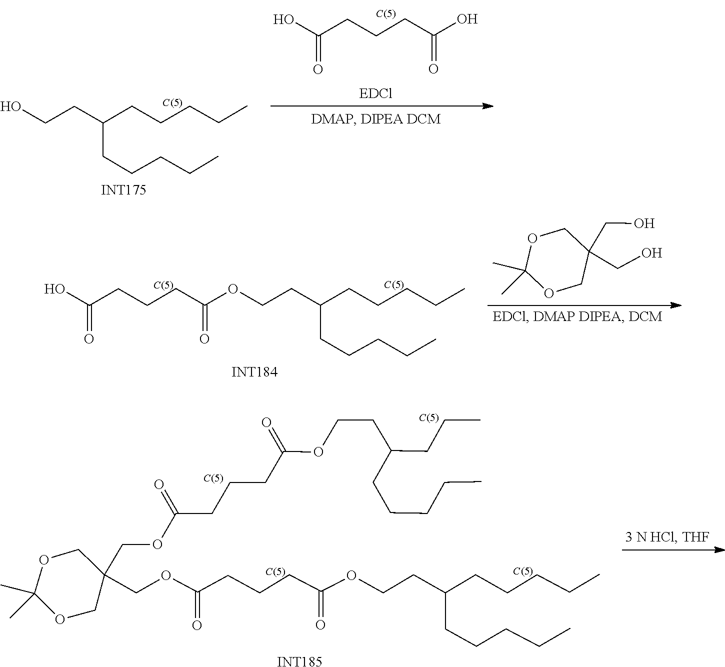

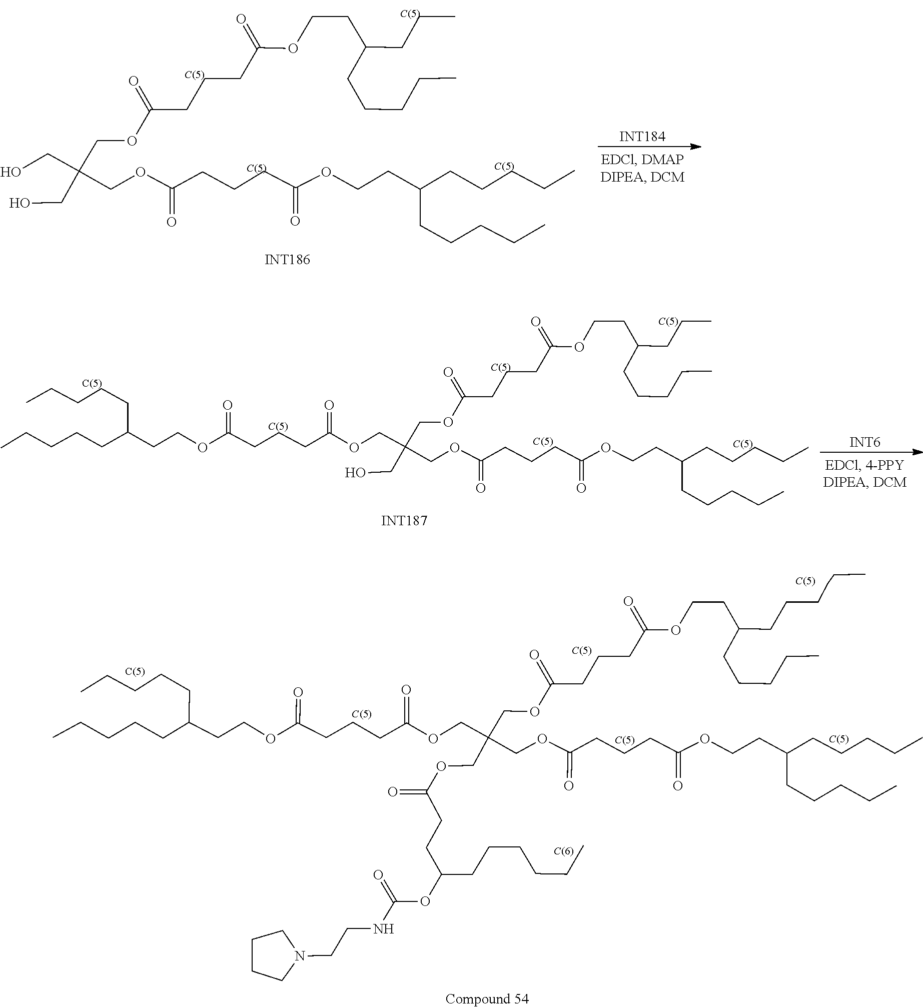

### Step 1:

To a solution of 3-pentyloctan-1-ol (10 g, 49.91 mmol, 1 eq) in DCM (250 mL) and THF (250 mL) was added EDCI (11.48 g, 59.89 mmol, 1.2 eq), DIPEA (16.13 g, 124.78 mmol, 21.73 mL, 2.5 eq), glutaric acid (32.97 g, 249.55 mmol, 5 eq) and DMAP (609.74 mg, 4.99 mmol, 0.1 eq) under N₂ atmosphere. The mixture was stirred at 25° C. for 12 hr under N₂ atmosphere. The reaction mixture was concentrated under reduced pressure to remove solvent. The residue was diluted with H₂O (100 mL) and extracted with DCM 300 mL (100 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound 5-oxo-5-(3-pentyloctoxy)pentanoic acid (10 g, 31.80 mmol, 63.72% yield) was obtained as a colorless oil. ¹H NMR (400 MHz, CDCl₃) δ ppm 4.10 (t, J=7.2 Hz, 2H), 2.42 (m, 4H), 1.97 (m, 2H), 1.58 (m, 2H), 1.40 (s, 1H), 1.19-1.36 (m, 16H), 0.89 (t, J=7.0 Hz, 6H).

### Step 2:

To a solution of 5-oxo-5-(3-pentyloctoxy) pentanoic acid (7.93 g, 25.22 mmol, 1 eq) in DCM (100 mL) was added EDCI (5.80 g, 30.27 mmol, 1.2 eq), DIPEA (8.15 g, 63.06 mmol, 10.98 mL, 2.5 eq), [5-(hydroxymethyl)-2,2-dimethyl-1,3-dioxan-5-yl]methanol (2 g, 11.35 mmol, 0.45 eq) and DMAP (308.14 mg, 2.52 mmol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (40 mL) and extracted with DCM 120 mL (40 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O5-[[2,2-dimethyl-5-[[5-oxo-5-(3-pentyloctoxy) pentanoyl]-oxymethyl]-1,3-dioxan-5-yl]methyl]O1-(3-pentyloctyl) pentanedioate (5 g, 6.50 mmol, 25.78% yield) was obtained as a colorless oil. ¹H NMR (400 MHz, CDCl₃) δ ppm 4.12-4.08 (m, 8H), 3.74 (s, 4H), 2.42-2.34 (m, 8H), 1.96-1.93 (m, 4H), 1.52-1.51 (m, 8H), 1.34 (s, 8H), 1.24-1.18 (m, 30H), 0.82 (t, J=3.2 Hz, 12H).

### Step 3:

To a solution of 05-[[2,2-dimethyl-5-[[5-oxo-5-(3-pentyloctoxy)pentanoyl]oxymethyl]-1,3-dioxan-5-yl]methyl]O1-(3-pentyloctyl) pentanedioate (2 g, 2.60 mmol, 1 eq) in THF (20 mL) was added HCl (3 M, 953.50 L, 1.1 eq) at 0° C. under N₂ atmosphere. The reaction mixture was stirred at 25° C. for 2 hr under N₂ atmosphere. The reaction mixture was diluted with aq. NaHCO₃ (40 mL) and extracted with EtOAc 120 mL (40 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound 05-[ 2,2-bis(hydroxymethyl)-3-[5-oxo-5-(3-pentyloctoxy)pentanoyl]oxy-propyl]O1-(3-pentyloctyl)pentanedioate (1 g, 1.37 mmol, 52.75% yield) was obtained as a colorless oil.

### Step 4:

To a solution of 05-[2,2-bis(hydroxymethyl)-3-[5-oxo-5-(3-pentyloctoxy)pentanoyl]oxy-propyl]O1-(3-pentyloctyl) pentanedioate (2 g, 2.74 mmol, 1 eq) in DCM (25 mL) was added EDCI (631.09 mg, 3.29 mmol, 1.2 eq), DIPEA (886.40 mg, 6.86 mmol, 1.19 mL, 2.5 eq), 5-oxo-5-(3-pentyloctoxy) pentanoic acid (517.60 mg, 1.65 mmol, 0.6 eq) and DMAP (33.51 mg, 274.34 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (50 mL) and extracted with DCM 180 mL (60 mL*3). The combined organic layers were filtered and the filtrate was concentrated under reduced pressure to give a residue. The residue was purified by column chromatography (SiO₂, Petroleum ether/Ethyl acetate=100/1 to 0/1). Compound O5-[2-(hydroxymethyl)-3-[5-oxo-5-(3-pentyloctoxy) pentanoyl]oxy-2-[[5-oxo-5-(3-pentyloctoxy)pentanoyl]oxy-methyl]propyl]O1-(3-pentyloctyl) pentanedioate (1 g, 975.16 mol, 35.55% yield) was obtained as a colorless oil.

### Step 5:

To a solution of 05-[2-(hydroxymethyl)-3-[5-oxo-5-(3-pentyloctoxy)pentanoyl]oxy-2-[[5-oxo-5-(3-pentyloctoxy)pentanoyl]oxymethyl]propyl]O1-(3-pentyloctyl) pentanedioate (1 g, 975.16 mol, 1 eq) in DCM (15 mL) was added EDCI (224.32 mg, 1.17 mmol, 1.2 eq), 4-pyrrolidin-1-ylpyridine (14.45 mg, 97.52 mol, 0.1 eq), 4-(2-pyrrolidin-1-ylethylcarbamoyloxy)decanoic acid (384.34 mg, 1.17 mmol, 1.2 eq) and DIPEA (315.07 mg, 2.44 mmol, 424.63 L, 2.5 eq) under N₂ atmosphere. The reaction mixture was stirred at 25° C. for 12 hr under N₂ atmosphere. The reaction mixture was diluted with H₂O (40 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O5-[2,2-bis[[5-oxo-5-(3-pentyloctoxy) pentanoyl]oxymethyl]-3-[4-(2-pyrrolidin-1-ylethylcarbamoyloxy)decanoyloxy]propyl]O1-(3-pentyloctyl) pentanedioate (0.1 g, 74.86 mol, 7.68% yield) was obtained as a colorless oil. ¹H NMR (400 MHz, CDCl₃) δ ppm 5.56-5.31 (m, 1H), 4.75 (s, 1H), 4.12-4.07 (m, 14H), 3.38 (s, 2H), 2.76 (s, 4H), 2.41-2.34 (m, 14H), 1.97-1.77 (m, 14H), 1.60-1.55 (m, 8H) 1.40 (s, 4H), 1.33-1.26 (m, 58H), 0.91-0.87 (m, 21H).

### Example 62—Compound 55

### [6-[2,2-bis[6-(2-butyloctanoyloxy)hexanoyloxymethyl]-3-[4-(2-pyrrolidin-1ylethylcarbamoyloxy)decanoyloxy]propoxy]-6-oxo-hexyl]2-butyloctanoate

### Synthetic Scheme:

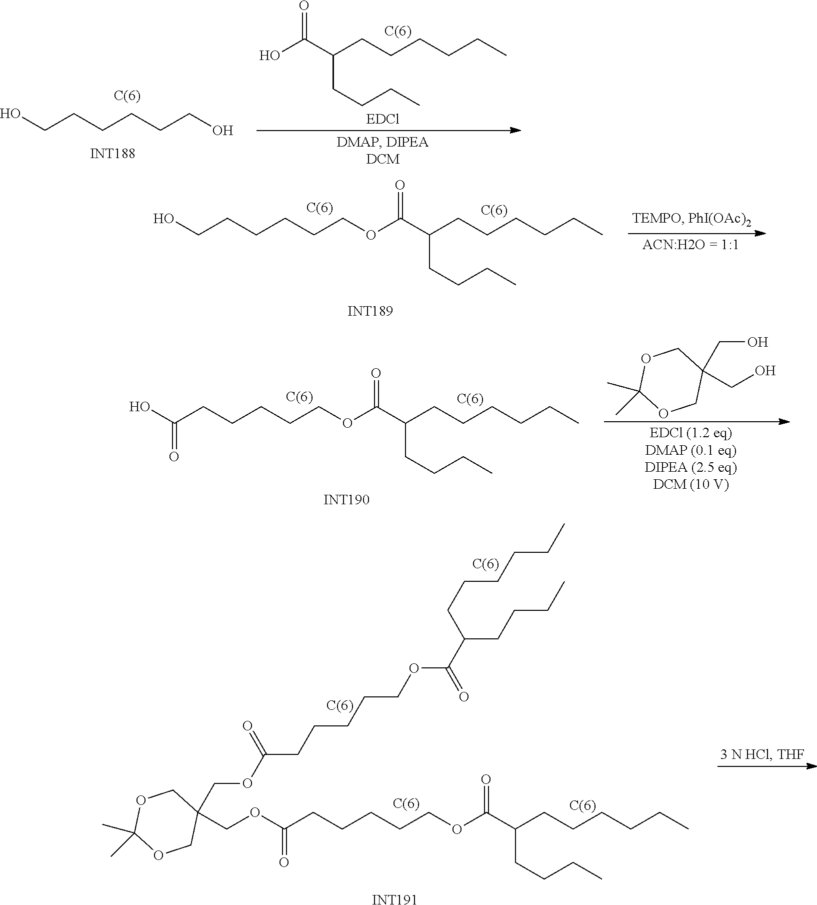

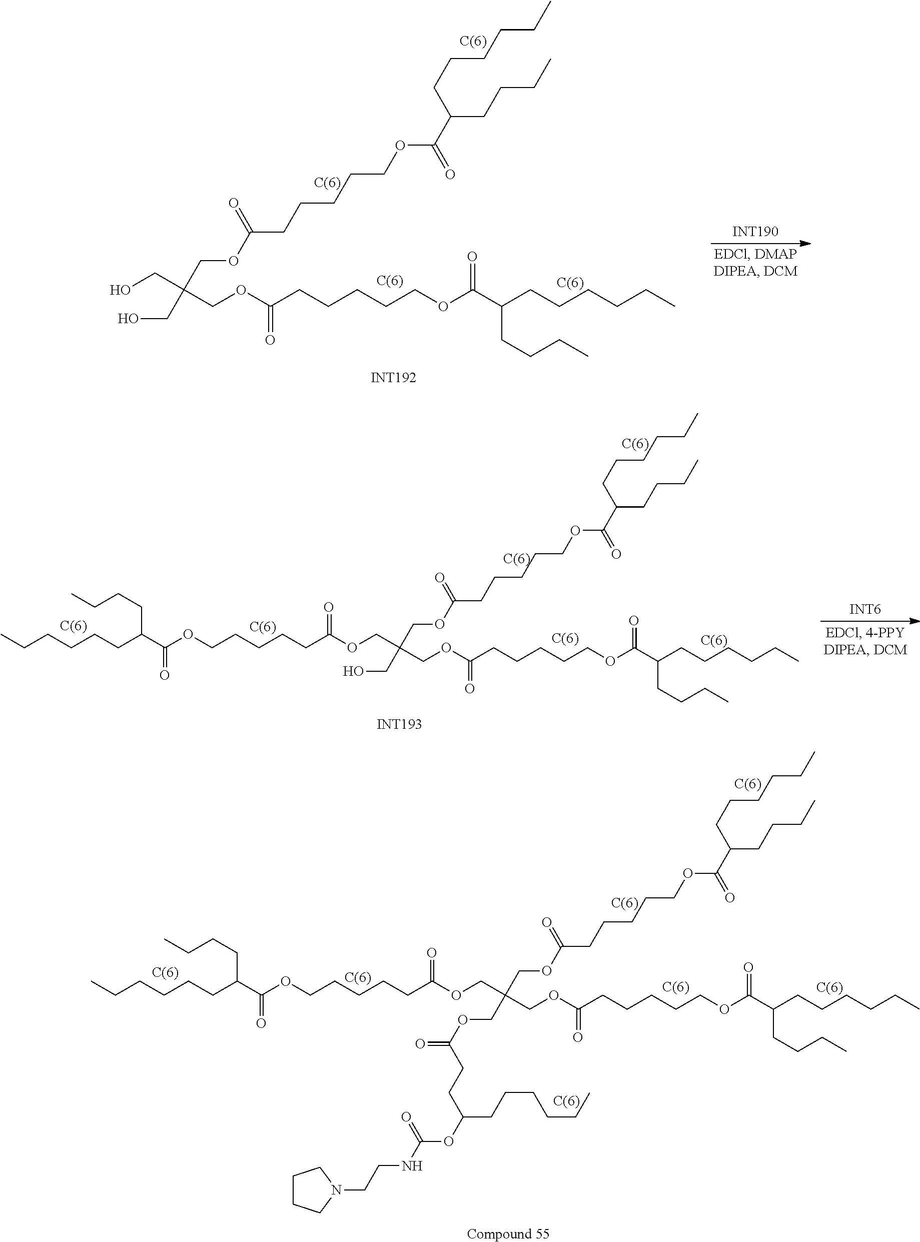

### Step 1:

To a solution of hexane-1,6-diol (4 g, 33.85 mmol, 4.04 mL, 1 eq) in DCM (340 mL) was added EDCI (9.73 g, 50.77 mmol, 1.5 eq), DIPEA (13.12 g, 101.55 mmol, 17.69 mL, 3 eq) and DMAP (2.07 g, 16.92 mmol, 0.5 eq) under N₂ and then 2-butyloctanoic acid (2.71 g, 13.54 mmol, 0.4 eq) was added to the above reaction mixture. The reaction mixture was stirred at 25° C. for 12 hr under N₂ atmosphere. The reaction mixture was concentrated under reduced pressure to remove solvent. The residue was diluted with water (150 mL) and extracted with DCM 450 mL (150 mL*3). The combined organic layers were washed with brine (150 mL), dried over Na₂SO₄, filtered and the filtrate was concentrated under reduced pressure to give a residue. The residue was purified by column chromatography (SiO₂, Petroleum ether/Ethyl acetate=10/1). Compound 6-hydroxyhexyl 2-butyloctanoate (13 g, 43.26 mmol, 25.56% yield) was obtained as a colourless oil. (¹H NMR (400 MHz, CDCl₃) δ ppm 4.08 (t, J=6.6 Hz, 2H), 2.38 (t, J=7.6 Hz, 2H), 1.79-1.49 (m, 7H), 1.47-1.38 (m, 4H), 1.36-1.18 (m, 14H), 0.88 (m, 6H).

### Step 2:

To a solution of 6-hydroxyhexyl 2-butyloctanoate (13 g, 43.26 mmol, 1 eq) in ACN (150 mL) and H₂O (150 mL) was added 1-oxidanyl-2,2,6,6-tetramethyl-piperidine (4.08 g, 25.96 mmol, 0.6 eq) and [acetoxy(phenyl)-iodanyl]acetate (34.84 g, 108.16 mmol, 2.5 eq) under N₂ atmosphere. The reaction mixture was stirred at 25° C. for 12 hr under N₂ atmosphere. The reaction mixture was concentrated under reduced pressure to remove solvent. The residue was diluted with water (300 mL) and extracted with ethyl acetate 450 mL (150 mL*3). The combined organic layers were washed with brine (300 mL), dried over Na₂SO₄, filtered and the filtrate was concentrated under reduced pressure to give a residue. The residue was purified by column chromatography (SiO₂, Petroleum ether/Ethyl acetate=5/1). Compound 6-(2-butyloctanoyloxy)hexanoic acid (7 g, 22.26 mmol, 51.45% yield) was obtained as a colourless oil.

### Step 3:

To a solution of 6-(2-butyloctanoyloxy)hexanoic acid (3.97 g, 12.61 mmol, 1 eq) in DCM (40 mL) was added DIPEA (4.07 g, 31.53 mmol, 5.49 mL, 2.5 eq), EDCI (2.90 g, 15.13 mmol, 1.2 eq) and DMAP (154.07 mg, 1.26 mmol, 0.1 eq) under N₂ and then the solution of [5-(hydroxymethyl)-2,2-dimethyl-1,3-dioxan-5-yl]methanol (1 g, 5.68 mmol, 0.45 eq) in DCM (10 mL) was added to the above reaction mixture. The reaction mixture was stirred at 25° C. for 12 hr under N₂ atmosphere. The reaction mixture was concentrated under reduced pressure to remove solvent. The residue was diluted with water (300 mL) and extracted with DCM 750 mL (250 mL*3). The combined organic layers were washed with brine (300 mL), dried over Na₂SO₄, filtered and the filtrate was concentrated under reduced pressure to give a residue. The residue was purified by column chromatography (SiO₂, Petroleum ether/Ethyl acetate=5/1). Compound [6-[[5-[6-(2-butyloctanoyloxy)hexanoyloxymethyl]-2,2-dimethyl-1,3-dioxan-5-yl]methoxy]-6-oxo-hexyl]2-butyloctanoate (7 g, 9.10 mmol, 14.43% yield) was obtained as a colourless oil. ¹H NMR (400 MHz, CDCl₃) δ ppm 4.11-4.05 (m, 8H), 3.75 (s, 4H), 2.40-2.27 (m, 6H), 1.72-1.55 (m, 16H), 1.48-1.38 (m, 12H), 1.32-1.22 (m, 22H), 0.88-0.86 (m, 12H).

### Step 4:

To a solution of [6-[[5-[6-(2-butyloctanoyloxy)hexanoyloxymethyl]-2,2-dimethyl-1,3-dioxan-5-yl]methoxy]-6-oxohexyl]2-butyloctanoate (1.5 g, 1.95 mmol, 1 eq) in THF (15 mL) was added HCl (3 M, 715.12 L, 1.1 eq) at 0° C. under N₂ atmosphere. The reaction mixture was stirred at 25° C. for 12 hr under N₂ atmosphere. The reaction mixture was diluted with sat. NaHCO₃ (20 mL) and extracted with ethyl acetate 60 mL (20 mL*3). The combined organic layers were washed with brine (20 mL), dried over Na₂SO₄, filtered and the filtrate was concentrated under reduced pressure to give a residue. The residue was purified by column chromatography (SiO₂, Petroleum ether/Ethyl acetate=1/1). Compound [6-[2-[6-(2-butyloctanoyloxy)hexanoyloxymethyl]-3-hydroxy-2-(hydroxymethyl)propoxy]-6-oxo-hexyl]2-butyloctan-oate (700 mg, 960.18 mol, 49.23% yield) was obtained as a colourless oil.

### Step 5:

To a solution of [6-[2-[6-(2-butyloctanoyloxy)hexanoyloxymethyl]-3-hydroxy-2-(hydroxymethyl)propoxy]-6-oxohexyl]2-butyloctanoate (3 g, 4.12 mmol, 1 eq) in DCM (30 mL) was added DIPEA (1.33 g, 10.29 mmol, 1.79 mL, 2.5 eq), EDCI (946.63 mg, 4.94 mmol, 1.2 eq) and DMAP (50.27 mg, 411.50 mol, 0.1 eq) under N₂ atmosphere. Then a solution of 6-(2-butyloctanoyloxy)hexanoic acid (776.41 mg, 2.47 mmol, 0.6 eq) in DCM (10 mL) was added to the above reaction mixture dropwise. The reaction mixture was stirred at 25° C. for 12 hr under N₂ atmosphere. The reaction mixture was concentrated under reduced pressure to remove solvent. The residue was diluted with water (40 mL) and extracted with DCM 60 mL (20 mL*3). The combined organic layers were washed with brine (20 mL), dried over Na₂SO₄, filtered and the filtrate was concentrated under reduced pressure to give a residue. The residue was purified by column chromatography (SiO₂, Petroleum ether/Ethyl acetate=2/1 to 1/1). Compound [6-[2,2-bis[6-(2-butyloctanoyloxy)hexanoyloxymethyl]-3-hydroxy-propoxy]-6-oxo-hexyl]2-butyloctanoate (1.6 g, 1.56 mmol, 37.92% yield) was obtained as a colourless oil.

### Step 6:

To a mixture of [6-[2,2-bis[6-(2-butyloctanoyloxy)hexanoyloxymethyl]-3-hydroxy-propoxy]-6-oxo-hexyl]2-butyloctanoate (880 mg, 858.14 mol, 1 eq), DIPEA (277.27 mg, 2.15 mmol, 373.68 L, 2.5 eq), EDCI (197.41 mg, 1.03 mmol, 1.2 eq) and 4-pyrrolidin-1-ylpyridine (12.72 mg, 85.81 mol, 0.1 eq) in DCM (10 mL) was added 4-(2-pyrrolidin-1-ylethylcarbamoyloxy)decanoic acid (338.22 mg, 1.03 mmol, 1.2 eq) and the reaction mixture was stirred at 25° C. for 12 hr under N₂ atmosphere. The reaction mixture was diluted with water (40 mL) and extracted with DCM 60 mL (20 mL*3). The combined organic layers were washed with brine (20 mL), dried over Na₂SO₄, filtered and the filtrate was concentrated under reduced pressure to give a residue. The residue was purified by column chromatography (SiO₂, Petroleum ether/Ethyl acetate=1/1). Compound [6-[2,2-bis[6-(2-butyloctanoyloxy)hexanoyloxymethyl]-3-[4-(2-pyrrolidin- 1ylethylcarbamoyloxy)decanoyloxy]propoxy]-6-oxo-hexyl]2-butyloctanoate (120 mg, 89.83 mol, 10.47% yield) was obtained as a colorless oil. ¹H NMR (400 MHz, CDCl₃) δ ppm 5.27 (s, 1H), 4.75 (br s, 1H), 4.18-4.03 (m, 14H), 3.31 (d, J=5.0 Hz, 2H), 2.70-2.48 (m, 6H), 2.41-2.27 (m, 11H), 1.98-1.74 (m, 8H), 1.71-1.56 (m, 20H), 1.49-1.35 (m, 14H), 1.31-1.22 (m, 40H), 0.88-0.86 (m, 21H).

### Example 63—Compound 56

### [5-[2,2-bis[5-(2-butyloctanoyloxy)pentanoyloxymethyl]-3-[4-(2-pyrrolidin-1-ylethylcarbamoyloxy)decanoyloxy]propoxy]-5-oxo-pentyl]2-butyloctanoate

### Synthetic Scheme:

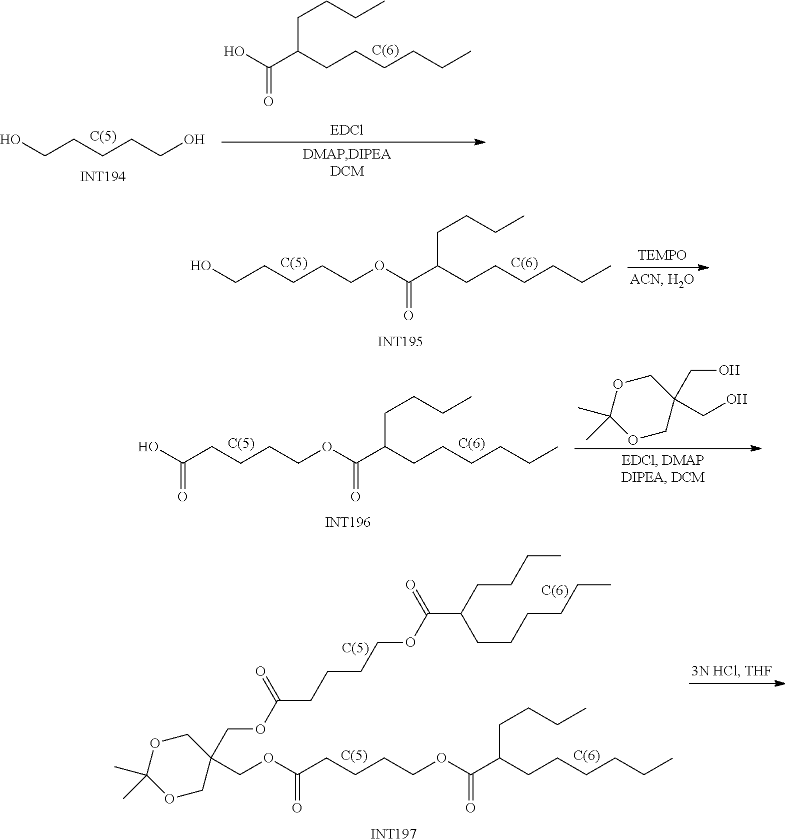

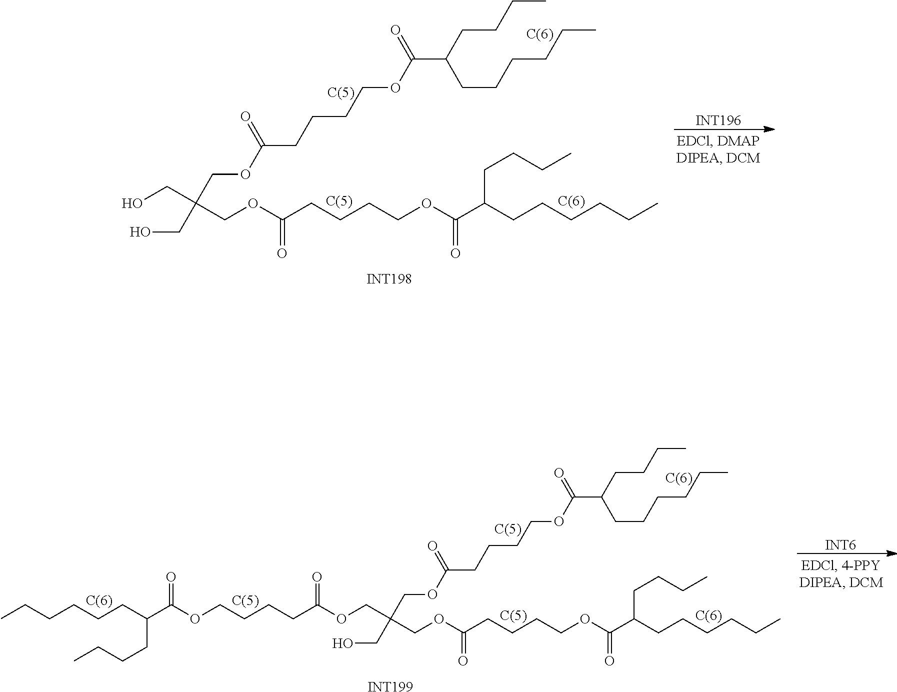

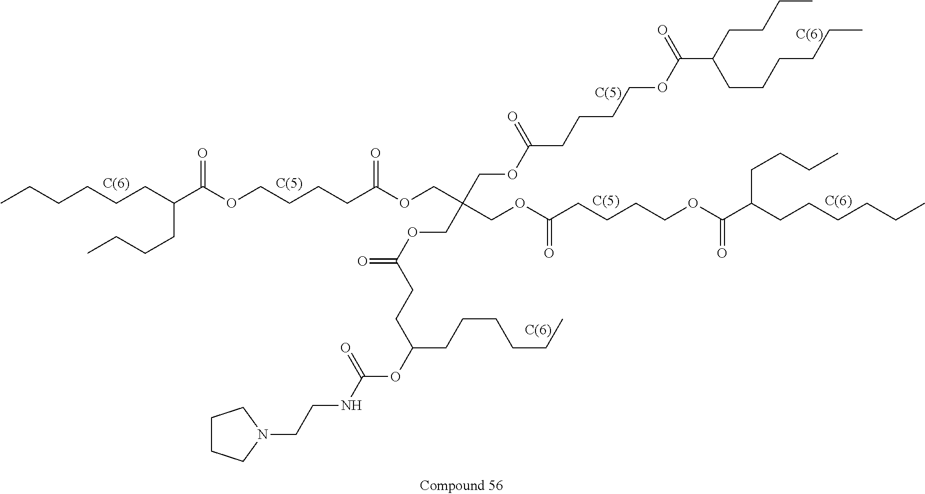 [SMILES: CCCCCCC(CCC(=O)OCC(COC(=O)CCCCOC(=O)C(CCCC)CCCCCC)(COC(=O)CCCCOC(=O)C(CCCC)CCCCCC)COC(=O)CCCCOC(=O)C(CCCC)CCCCCC)OC(=O)NCCN1CCCC1]

### Step 1:

To a solution of pentane-1,5-diol (19.50 g, 187.20 mmol, 19.69 mL, 1 eq) in DCM (345 mL) was added EDCI (43.06 g, 224.64 mmol, 1.2 eq), 2-butyloctanoic acid (15 g, 74.88 mmol, 0.4 eq), DMAP (2.29 g, 18.72 mmol, 0.1 eq) and DIPEA (60.49 g, 468.01 mmol, 81.52 mL, 2.5 eq) under N₂ atmosphere. The reaction mixture was stirred at 25° C. for 12 hr under N₂ atmosphere. The reaction mixture was diluted with H₂O (500 mL) and extracted with DCM 1000 mL (500 mL*2). The combined organic layers were dried over Na₂SO₄, filtered and the filtrate was concentrated under reduced pressure to give a residue. Compound 5-hydroxypentyl 2-butyloctanoate (17 g, 59.35 mmol, 31.70% yield) was obtained as a colorless oil. ¹H NMR (400 MHz, CDCl₃) δ ppm 4.09 (t, J=6.6 Hz, 2H), 3.66 (t, J=6.6 Hz, 2H), 2.32 (m, 1H), 1.54-1.74 (m, 6H), 1.42-1.48 (m, 4H), 1.19-1.36 (m, 12H), 0.88 (m, 6H).

### Step 2:

To a solution of 5-hydroxypentyl 2-butyloctanoate (17 g, 59.35 mmol, 1 eq) in ACN (85 mL) and H₂O (85 mL) was added [acetoxy (phenyl)-iodanyl]acetate (47.79 g, 148.37 mmol, 2.5 eq) and TEMPO (5.60 g, 35.61 mmol, 0.6 eq) under N₂ atmosphere. The reaction mixture was stirred at 25° C. for 12 hr under N₂ atmosphere. The reaction mixture was diluted with H₂O (500 mL) and extracted with EtOAc 1000 mL (500 mL*2). The combined organic layers were dried over Na₂SO₄, filtered and the filtrate was concentrated under reduced pressure to give a residue. Compound 5-(2-butyloctanoyloxy)pentanoic acid (14 g, 46.60 mmol, 78.52% yield) was obtained as a colorless oil. ¹H NMR (400 MHz, CDCl₃) δ ppm 4.10 (t, J=6.0 Hz, 2H), 2.41 (t, J=3.6 Hz, 2H), 2.36-2.29 (m, 1H), 1.76-1.71 (m, 4H), 1.64-1.55 (m, 2H), 1.49-1.40 (m, 2H), 1.34-1.20 (m, 12H), 0.90-0.86 (m, 6H).

### Step 3:

To a solution of 5-(2-butyloctanoyloxy)pentanoic acid (22.73 g, 75.67 mmol, 1 eq) in DCM (280 mL) was added EDCI (17.41 g, 90.80 mmol, 1.2 eq), [5-(hydroxymethyl)-2,2-dimethyl-1,3-dioxan-5-yl]methanol (6 g, 34.05 mmol, 0.45 eq), DMAP (924.41 mg, 7.57 mmol, 0.1 eq) and DIPEA (24.45 g, 189.17 mmol, 32.95 mL, 2.5 eq) under N₂ atmosphere. The reaction mixture was stirred at 25° C. for 12 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column. Compound [5-[[5-[5-(2-butyloctanoyloxy) pentanoyloxymethyl]-2,2-dimethyl-1,3-dioxan-5-yl]methoxy]-5-oxo-pentyl]2-butyloctanoate (10 g, 13.49 mmol, 17.83% yield) was obtained as a colorless oil. ¹H NMR (400 MHz, CDCl₃) δ ppm 4.12 (s, 4H), 4.09 (t, J=6.0 Hz, 4H), 3.74 (s, 4H), 2.39-2.29 (m, 6H), 1.73-1.66 (m, 8H), 1.64-1.55 (m, 6H), 1.49-1.40 (m, 10H), 1.34-1.21 (m, 24H), 0.90-0.86 (m, 12H).

### Step 4:

To a solution of [5-[[5-[5-(2-butyloctanoyloxy)pentanoyloxymethyl]-2,2-dimethyl-1,3-dioxan-5-yl]methoxy]-5-oxo-pentyl]2-butyloctanoate (10 g, 13.49 mmol, 1 eq) in THE (100 mL) was added HCl (3 M, 4.95 mL, 1.1 eq) at 0° C. under N₂ atmosphere. The reaction mixture was stirred at 25° C. for 2 hr under N₂ atmosphere. The reaction mixture was diluted with sat. NaHCO₃ (100 mL) and extracted with EtOAc 200 mL (50 mL*4). The combined organic layers were dried over Na₂SO₄, filtered and the filtrate was concentrated under reduced pressure to give a residue. Compound [5-[2-[5-(2-butyloctanoyloxy) pentanoyloxymethyl]-3-hydroxy-2-(hydroxymethyl)propoxy]-5-oxo-pentyl]2-butyloctanoate (6 g, 8.56 mmol, 63.43% yield) was obtained as a colorless oil. ¹H NMR (400 MHz, CDCl₃) δ ppm 4.15 (s, 4H), 4.10 (t, J=8.8 Hz, 4H), 3.59 (s, 4H), 2.79-2.61 (s, 1H), 2.41 (t, J=3.4 Hz, 4H), 2.35-2.28 (m, 2H), 1.74-1.54 (m, 12H), 1.19-1.40 (m, 4H), 1.34-1.21 (m, 24H), 0.90-0.86 (m, 12H).

### Step 5:

To a solution of [5-[2-[5-(2-butyloctanoyloxy)pentanoyloxymethyl]-3-hydroxy-2-(hydroxymethyl)propoxy]-5-oxo-pentyl]2-butyloctanoate (3 g, 4.28 mmol, 1 eq) in DCM (37 mL) was added EDCI (984.51 mg, 5.14 mmol, 1.2 eq), 5-(2-butyloctanoyloxy)pentanoic acid (771.46 mg, 2.57 mmol, 0.6 eq), DMAP (52.28 mg, 427.97 mol, 0.1 eq) and DIPEA (1.38 g, 10.70 mmol, 1.86 mL, 2.5 eq) under N₂ atmosphere. The reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. Compound [5-[2,2-bis[5-(2-butyloctanoyloxy) pentanoyloxymethyl]-3-hydroxy-propoxy]-5-oxo-pentyl]2-butyloctanoate (1.5 g, 1.53 mmol, 35.64% yield) was obtained as a colorless oil. ¹H NMR (400 MHz, CDCl₃) δ ppm 4.13 (s, 6H), 4.09 (t, J=6.0 Hz, 6H), 3.51 (s, 2H), 2.39 (t, J=7.0 Hz, 6H), 2.32 (m, 3H), 1.71-1.65 (m, 10H), 1.62-1.55 (m, 10H), 1.49-1.39 (m, 6H), 1.33-1.23 (m, 34H), 0.88 (m, 18H).

### Step 6:

To a solution of [5-[2,2-bis[5-(2-butyloctanoyloxy)pentanoyloxymethyl]-3-hydroxy-propoxy]-5-oxo-pentyl]2-butyloctanoate (1.5 g, 1.53 mmol, 1 eq) in DCM (19 mL) was added EDCI (350.89 mg, 1.83 mmol, 1.2 eq), 4-(2-pyrrolidin-1-ylethylcarbamoyloxy)decanoic acid (450.89 mg, 1.37 mmol, 0.9 eq), 4-pyrrolidin-1-ylpyridine (22.61 mg, 152.53 mol, 0.1 eq) and DIPEA (492.84 mg, 3.81 mmol, 664.21 L, 2.5 eq) under N₂. The reaction mixture was stirred at 25° C. for 12 hr under N₂ atmosphere. The reaction mixture was diluted with H₂O (50 mL) and extracted with DCM 50 mL (25 mL*2). The combined organic layers were dried over Na₂SO₄, filtered and the filtrate was concentrated under reduced pressure to give a residue. Compound [5-[2,2-bis[5-(2-butyloctanoyloxy) pentanoyloxymethyl]-3-[4-(2-pyrrolidin-1-ylethylcarbamoyloxy)decanoyloxy]propoxy]-5-oxo-pentyl]2-butyloctanoate (0.12 g, 92.75 mol, 6.08% yield) was obtained as a colorless oil. ¹H NMR (400 MHz, CDCl₃) δ ppm 5.37-5.05 (s, 1H), 4.75 (s, 1H), 4.15-4.07 (m, 14H), 3.32-3.31 (s, 2H), 2.61 (d, J=12.2 Hz, 6H), 2.40-2.28 (m, 11H), 2.02 (s, 2H), 1.81-1.74 (s, 6H), 1.73-1.64 (m, 14H), 1.63-1.52 (m, 8H), 1.50-1.40 (m, 7H), 1.34-1.21 (m, 44H), 0.90-0.86 (m, 21H).

### Example 64—Compound 57

### [7-[2-[[2-(1-adamantyl)acetyl]oxymethyl]-2-[7-(2-butyloctanoyloxy)heptanoyloxymethyl]-3-[4-(2-pyrrolidin-1-ylethylcarbamoyloxy)decanoyloxy]propoxy]-7-oxo-heptyl]2-butyloctanoate

### Synthetic Scheme:

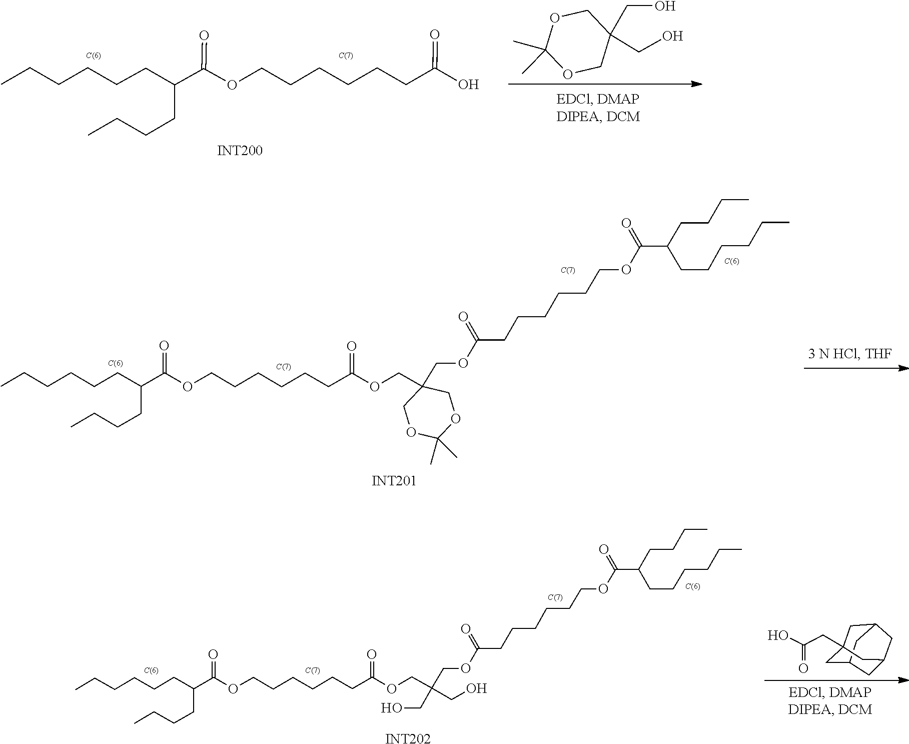

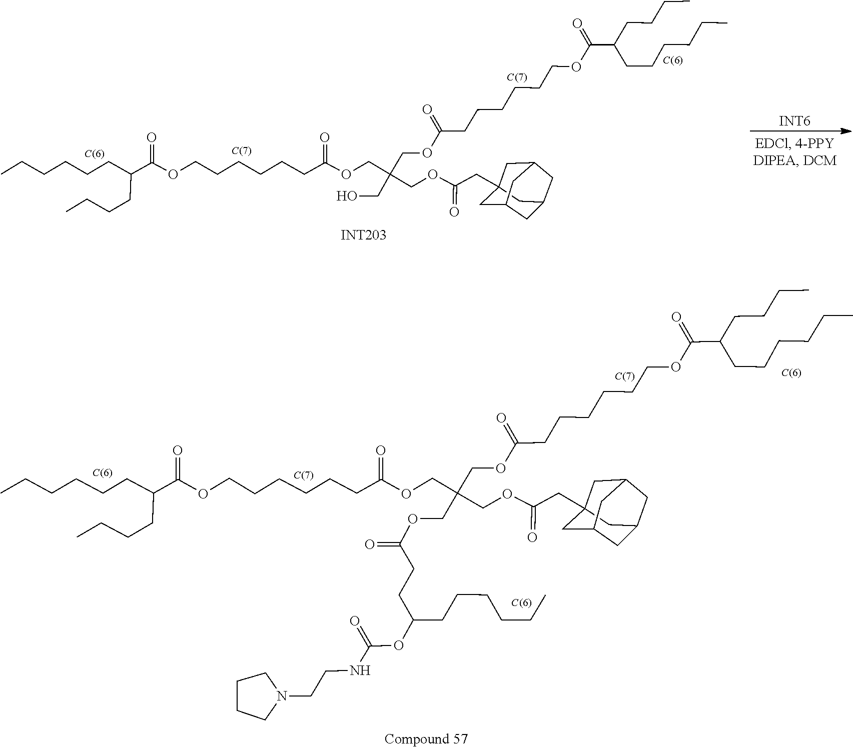

### Step 1:

To a mixture of [5-(hydroxymethyl)-2,2-dimethyl-1,3-dioxan-5-yl]methanol (1 g, 5.68 mmol, 0.45 eq), DMAP (154.07 mg, 1.26 mmol, 0.1 eq), EDCI (2.90 g, 15.13 mmol, 1.2 eq) and DIPEA (4.07 g, 31.53 mmol, 5.49 mL, 2.5 eq) in DCM (200 mL) was added 7-(2-butyloctanoyloxy)heptanoic acid (4.14 g, 12.61 mmol, 1 eq) under N₂ atmosphere and the reaction mixture was stirred at 25° C. for 12 hr under N₂ atmosphere. The reaction mixture was diluted with H₂O (200 mL) and extracted with DCM 600 mL (200 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound [7-[[5-[7-(2-butyloctanoyloxy)heptanoyloxymethyl]-2,2-dimethyl-1,3-dioxan-5-yl]methoxy]-7-oxo-heptyl]2-butyloctanoate (1.5 g, 1.88 mmol, 14.92% yield) was obtained as a colorless oil. ¹H NMR (400 MHz, CDCl₃) δ ppm 4.04 (s, 4H), 3.99 (d, J=3.4 Hz, 4H), 3.67 (s, 4H), 2.27-2.23 (m, 6H), 1.95-1.50 (m, 14H), 1.39-1.29 (m, 18H), 1.23-1.16 (m, 24H), 0.83-0.77 (m, 12H).

### Step 2:

To a solution of [7-[[5-[7-(2-butyloctanoyloxy)heptanoyloxymethyl]-2,2-dimethyl-1,3-dioxan-5-yl]methoxy]-7-oxo-heptyl]2-butyloctanoate (4.5 g, 5.65 mmol, 1 eq) in THE (45 mL) was added HCl (3 M, 2.07 mL, 1.1 eq) at 0° C. under N₂ atmosphere and the reaction mixture was stirred at 25° C. for 12 hr under N₂ atmosphere. The reaction mixture was diluted with H₂O (20 mL) and extracted with ethyl acetate 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound [7-[2-[7-(2-butyloctanoyloxy)heptanoyloxymethyl]-3-hydroxy-2-(hydroxymethyl)propoxy]-7-oxo-heptyl]2-butyloctanoate (1.5 g, 1.98 mmol, 35.10% yield) was obtained as a colorless oil. ¹H NMR (400 MHz, CDCl₃) δ ppm 0.88 (m, 12H), 1.17-1.33 (m, 24H), 1.35-1.49 (m, 12H), 1.54-1.66 (m, 12H), 2.26-2.43 (m, 6H), 3.59 (s, 4H), 4.07 (t, J=6.6 Hz, 4H), 4.15 (s, 4H).

### Step 3:

To a mixture of [7-[2-[7-(2-butyloctanoyloxy)heptanoyloxymethyl]-3-hydroxy-2-(hydroxymethyl)propoxy]-7-oxo-heptyl]2-butyloctanoate (1.5 g, 1.98 mmol, 1 eq), DMAP (24.20 mg, 198.13 mol, 0.1 eq), EDCI (455.78 mg, 2.38 mmol, 1.2 eq) and DIPEA (640.17 mg, 4.95 mmol, 862.76 L, 2.5 eq) in DCM (20 mL) was added 2-(1-adamantyl)acetic acid (230.94 mg, 1.19 mmol, 0.6 eq) under N₂ atmosphere. The reaction mixture was stirred at 25 ° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound [7-[2-[[2-(1-adamantyl)acetyl]oxymethyl]-2-[7-(2-butyloctanoyloxy)heptanoyloxymethyl]-3-hydroxy-propoxy]-7-oxo-heptyl]2-butyloctanoate (0.8 g, 857.14 mol, 43.26% yield) was obtained as a colorless oil. ¹H NMR (400 MHz, CDCl₃) δ ppm 4.21-3.93 (m, 10H), 3.51 (s, 2H), 2.56 (s, 1H), 2.39-2.24 (m, 6H), 2.10 (s, 2H), 1.98 (s, 3H), 1.78-1.57 (m, 24H), 1.51-1.22 (m, 36H), 0.88 (m, 12H).

### Step 4:

To a mixture of [7-[2-[[2-(1-adamantyl)acetyl]oxymethyl]-2-[7-(2-butyloctanoyloxy)heptanoyloxymethyl]-3-hydroxy-propoxy]-7-oxo-heptyl]2-butyloctanoate (0.8 g, 857.14 mol, 1 eq), DIPEA (276.95 mg, 2.14 mmol, 373.24 L, 2.5 eq), EDCI (197.18 mg, 1.03 mmol, 1.2 eq) and 4-pyrrolidin-1-ylpyridine (12.70 mg, 85.71 mol, 0.1 eq) in DCM (10 mL) was added 4-(2-pyrrolidin-1-ylethylcarbamoyloxy)decanoic acid (337.83 mg, 1.03 mmol, 1.2 eq) under N₂ atmosphere. The reaction mixture was stirred at 25° C. for 12 hr under N₂ atmosphere. The reaction mixture was diluted with H₂O (20 mL) and extracted with DCM 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). The residue was purified by column chromatography (SiO₂, Petroleum ether/Ethyl acetate=100/1 to 0/1). Compound [7-[2-[[2-(1-adamantyl)acetyl]oxymethyl]-2-[7-(2-butyloctanoyloxy)heptanoyloxymethyl]-3-[4-(2-pyrrolidin-1-ylethylcarbamoyloxy)decanoyloxy]propoxy]-7-oxo-heptyl]2-butyloctanoate (0.1 g, 80.16 mol, 9.35% yield, 99.7% purity) was obtained as a colorless oil. ¹H NMR (400 MHz, CDCl₃) δ ppm 4.75 (d, J=3.6 Hz, 1H), 4.20-3.98 (m, 12H), 3.57-3.24 (m, 2H), 2.80-2.68 (m, 2H), 2.41-2.35 (m, 2H), 2.35-2.28 (m, 6H), 2.08 (s, 2H), 1.97 (s, 3H), 1.89 (s, 2H), 1.75-1.68 (m, 4H), 1.66-1.56 (m, 34H), 1.49-1.39 (m, 6H), 1.39-1.35 (m, 6H), 1.32-1.23 (m, 30H), 0.88 (m, 15H).

### Example 65—Compound 58

### [6-[2-[[2-(1-adamantyl)acetyl]oxymethyl]-2-[6-(2-butyloctanoyloxy)hexanoyloxymethyl]-3-[4-(2-pyrolidin-1-ylethylcarbamoyloxy)decanoyloxy]propoxy]-6-oxo-hexyl]2-butyloctanoate

### Synthetic Scheme:

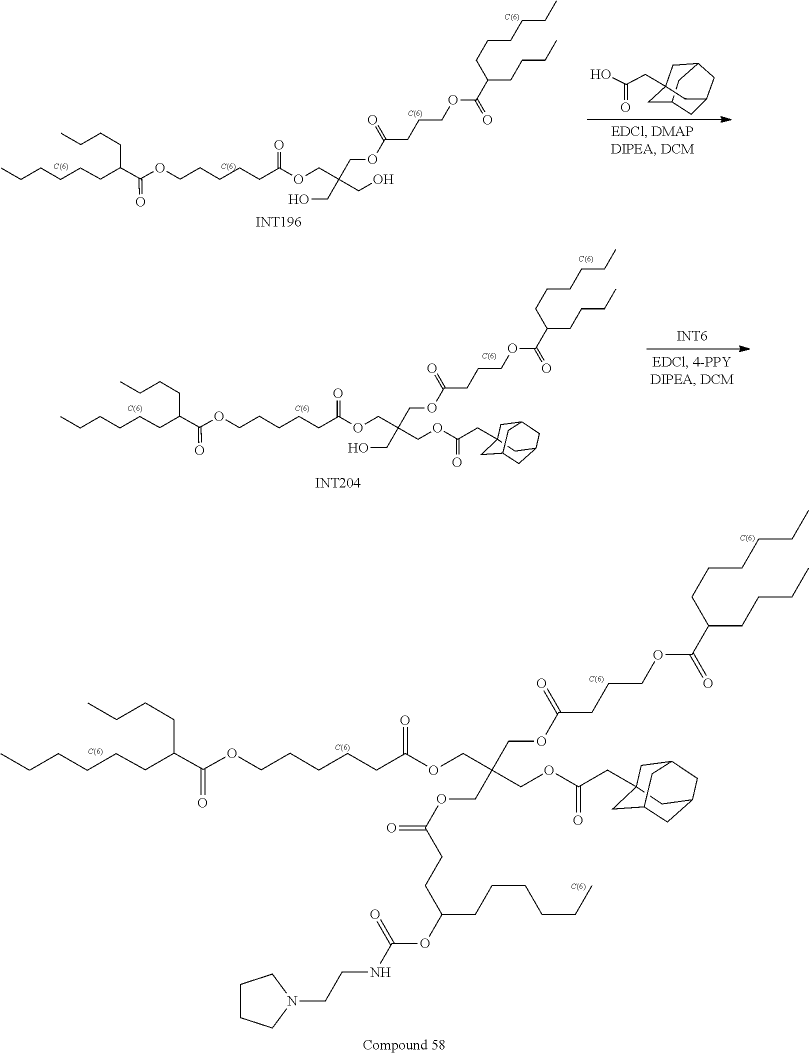

### Step 1:

To a solution of [6-[2-[6-(2-butyloctanoyloxy)hexanoyloxymethyl]-3-hydroxy-2-(hydroxymethyl)propoxy]-6-oxohexyl]2-butyloctanoate (800 mg, 1.10 mmol, 1 eq) in DCM (8 mL) was added DIPEA (354.56 mg, 2.74 mmol, 477.84 L, 2.5 eq), EDCI (252.43 mg, 1.32 mmol, 1.2 eq) and DMAP (13.41 mg, 109.73 mol, 0.1 eq) under N₂ atmosphere. Then a solution of 2-(1-adamantyl)acetic acid (127.91 mg, 658.41 mol, 0.6 eq) in DCM (2 mL) was added to the above reaction mixture. The reaction mixture was stirred at 25° C. for 12 hr under N₂ atmosphere. The reaction mixture was concentrated under reduced pressure to remove solvent. The residue was diluted with water (10 mL) and extracted with DCM 30 mL (10 mL*3). The combined organic layers were washed with brine (10 mL), dried over Na₂SO₄, filtered and the filtrate was concentrated under reduced pressure to give a residue. The residue was purified by column chromatography (SiO₂, Petroleum ether/Ethyl acetate=2/1 to 1/1). Compound [6-[2-[[2-(1-adamantyl)acetyl]oxymethyl]-2-[6-(2-butyloctanoyloxy)hexanoyloxymethyl]-3-hydroxypropoxy]-6-oxo-hexyl]2-butyloctanoate (400 mg, 441.85 mol, 40.27% yield) was obtained as a colorless oil.

### Step 2:

To a solution of [6-[2-[[2-(1-adamantyl)acetyl]oxymethyl]-2-[6-(2-butyloctanoyloxy)hexanoyloxymethyl]-3-hydroxypropoxy]-6-oxo-hexyl]2-butyloctanoate (400 mg, 441.85 mol, 1 eq) in DCM (10 mL) was added DIPEA (142.76 mg, 1.10 mmol, 192.41 μL, 2.5 eq), EDCI (101.64 mg, 530.22 mol, 1.2 eq) and 4-pyrrolidin-1-ylpyridine (6.55 mg, 44.18 mol, 0.1 eq) under N₂ atmosphere. Then a solution of 4-(2-pyrrolidin-1-ylethylcarbamoyloxy)decanoic acid (87.07 mg, 265.11 mol, 0.6 eq) in DCM (10 mL) was added to the above reaction mixture. The reaction mixture was stirred at 25° C. for 12 hr under N₂ atmosphere. The reaction mixture was concentrated under reduced pressure to remove solvent. The residue was diluted with water (10 mL) and extracted with DCM 30 mL (10 mL*3). The combined organic layers were washed with brine (10 mL), dried over Na₂SO₄, filtered and the filtrate was concentrated under reduced pressure to give a residue. The residue was purified by column chromatography (SiO₂, Petroleum ether/Ethyl acetate=5/1 to 3/1). Compound [6-[2-[[2-(1-adamantyl)acetyl]oxymethyl]-2-[6-(2-butyloctanoyloxy)hexanoyloxymethyl]-3-[4-(2-pyrolidin-1-ylethylcarbamoyloxy)decanoyloxy]propoxy]-6-oxo-hexyl]2-butyloctanoate (130 mg, 106.93 mol, 24.20% yield) was obtained as a colourless oil. ¹H NMR (400 MHz, CDCl₃) δ ppm 5.43 (s, 1H), 4.76 (s, 1H), 4.18-4.00 (m, 12H), 3.36 (s, 2H), 2.72 (s, 2H), 2.44-2.27 (m, 8H), 2.08 (s, 2H), 1.97 (s, 3H), 1.92-1.77 (m, 6H), 1.73-1.55 (m, 32H), 1.47-1.37 (m, 8H), 1.32-1.23 (m, 30H), 0.88 (m, 15H).

### Example 66—Compound 59

### [5-[2-[[2-(1-adamantyl) acetyl]oxymethyl]-2-[5-(2-butyloctanoyloxy) pentanoyloxymethyl]-3-[4-(2-pyrrolidin-1-ylethylcarbamoyloxy) decanoyloxy]propoxy]-5-oxo-pentyl]2-butyloctanoate

### Synthetic Scheme:

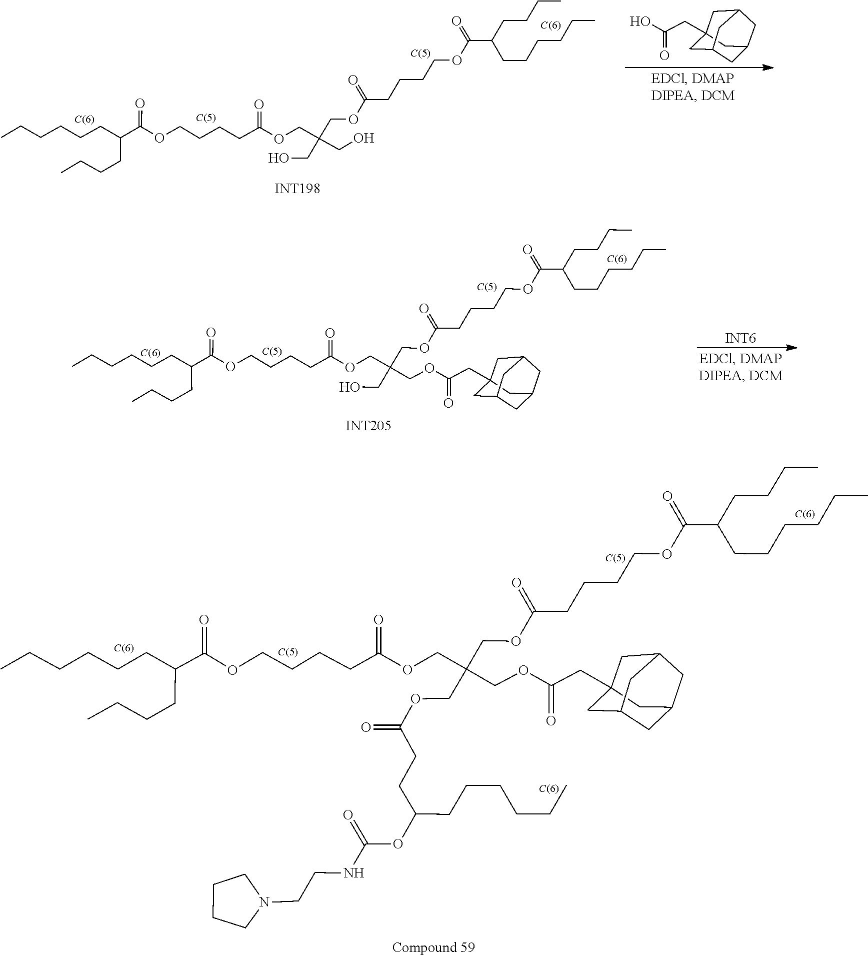

### Step 1:

To a solution of [5-[2-[5-(2-butyloctanoyloxy)pentanoyloxymethyl]-3-hydroxy-2-(hydroxymethyl)propoxy]-5-oxo-pentyl]2-butyloctanoate (4 g, 5.71 mmol, 1 eq) in DCM (48 mL) was added EDCI (1.31 g, 6.85 mmol, 1.2 eq), DMAP (69.71 mg, 570.63 mol, 0.1 eq), 2-(1-adamantyl)acetic acid (886.85 mg, 4.57 mmol, 0.8 eq) and DIPEA (1.84 g, 14.27 mmol, 2.48 mL, 2.5 eq) under N₂ atmosphere. The reaction mixture was stirred at 25° C. for 12 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column. Compound [5-[2-[[2-(1-adamantyl) acetyl]oxymethyl]-2-[5-(2-butyloctanoyloxy) pentane-yloxymethyl]-3-hydroxy-propoxy]-5-oxo-pentyl]2-butyloctanoate (1.5 g, 1.71 mmol, 29.97% yield) was obtained as a colorless oil. ¹H NMR (400 MHz, CDCl₃) δ ppm 4.13 (s, 4H), 4.10-4.07 (m, 6H), 3.52 (s, 2H), 2.41-2.30 (m, 6H), 2.10 (s, 2H), 1.97 (s, 3H), 1.73-1.55 (m, 28H), 1.49-1.41 (m, 4H), 1.33-1.231 (m, 24H), 0.90-0.86 (m, 12H).

### Step 2:

To a solution of [5-[2-[[2-(1-adamantyl)acetyl]oxymethyl]-2-[5-(2-butyloctanoyloxy)pentanoyloxymethyl]-3-hydroxy-propoxy]-5-oxo-pentyl]2-butyloctanoate (1.5 g, 1.71 mmol, 1 eq) in DCM (21 mL) was added EDCI (393.35 mg, 2.05 mmol, 1.2 eq), 4-(2-pyrrolidin-1-ylethylcarbamoyloxy)decanoic acid (673.94 mg, 2.05 mmol, 1.2 eq), DMAP (20.89 mg, 170.99 mol, 0.1 eq) and DIPEA (552.49 mg, 4.27 mmol, 744.59 L, 2.5 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column. Compound [5-[2-[[2-(1-adamantyl) acetyl]oxymethyl]-2-[5-(2-butyloctanoyloxy) pentanoyloxymethyl]-3-[4-(2-pyrrolidin-1-ylethylcarbamoyloxy) decanoyloxy]propoxy]-5-oxo-pentyl]2-butyloctanoate (0.1 g, 84.20 mol, 4.92% yield) was obtained as a colorless oil. ¹H NMR (400 MHz, CDCl₃) δ ppm 5.3 (s, 1H), 4.75 (s, 1H), 4.15-4.04 (m, 12H), 3.33-3.32 (s, 2H), 2.63 (d, J=11.6 Hz, 6H), 2.40-2.27 (m, 8H), 2.08 (s, 2H), 1.97 (s, 3H), 1.81 (s, 6H), 1.72-1.67 (m, 12H), 1.63-1.055 (m, 14H), 1.49-1.41 (m, 6H), 1.32-1.22 (m, 32H), 0.90-0.86 (m, 15H).

### Example 67—Compound 60

### O8-[2-[5-(dimethylamino)pentanoyloxymethyl]-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate

### Synthetic Scheme:

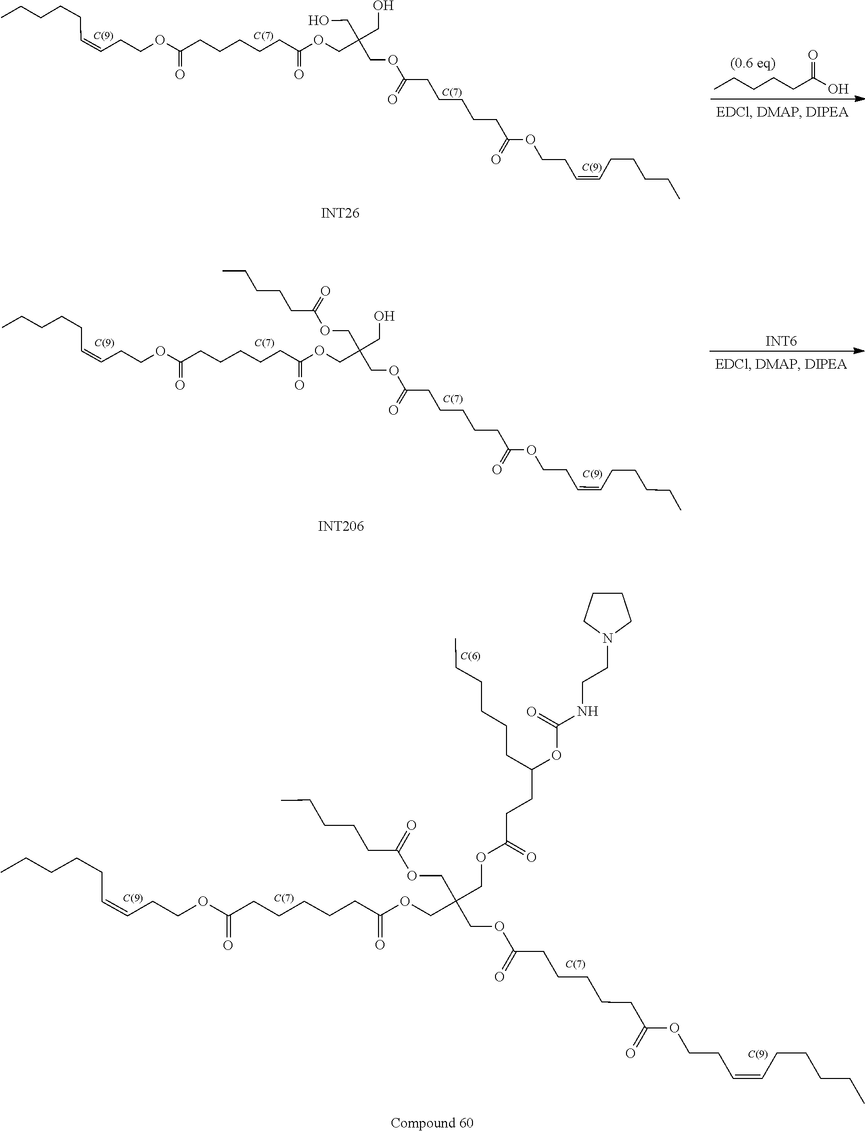

### Step 1:

To a solution of O7-[2,2-bis(hydroxymethyl)-3-[7-[(Z)-non-3-enoxy]-7-oxo-heptanoyl]oxy-propyl]O1-[(Z)-non-3-enyl]heptanedioate (3.5 g, 5.23 mmol, 1 eq), EDCI (1.20 g, 6.28 mmol, 1.2 eq), DMAP (63.93 mg, 523.25 mol, 0.1 eq) and DIPEA (1.69 g, 13.08 mmol, 2.28 mL, 2.5 eq) in DCM (40 mL) was added hexanoic acid (364.68 mg, 3.14 mmol, 393.40 μL, 0.6 eq) in DCM (30 mL) under N₂ atmosphere. The reaction mixture was stirred at 20° C. for 12 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=50/1 to 10/1). Compound O7-[2-(hexanoyloxymethyl)-2-(hydroxymethyl)-3-[7-[(Z)-non-3-enoxy]-7-oxo-hepta-noyl]oxy-propyl]O1-[(Z)-non-3-enyl]heptanedioate (1.5 g, 1.96 mmol, 37.37% yield) was obtained as a colorless oil. ¹H NMR (400 MHz, CDCl₃) δ ppm 5.57-5.45 (m, 2H), 5.40-5.29 (m, 2H), 4.18-4.02 (m, 10H), 3.51 (s, 2H), 2.41-2.29 (m, 14H), 2.08-2.00 (m, 4H), 1.68-1.61 (m, 10H), 1.41-1.27 (m, 20H), 0.96-0.82 (m, 9H).

### Step 2:

A mixture of O8-[2-(hydroxymethyl)-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (2 g, 2.05 mmol, 1 eq), 5-(dimethylamino)pentanoic acid (445.69 mg, 3.07 mmol, 1.2 eq), EDCI (470.74 mg, 2.46 mmol, 1.2 eq), DIPEA (661.19 mg, 5.12 mmol, 891.09 L, 2.5 eq) and DMAP (25.00 mg, 204.64 mol, 0.1 eq) in DCM (20 mL) was stirred at 20° C. for 12 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. Compound O8-[2-[5-(dimethylamino)pentanoyloxymethyl]-3-[8-[(Z)-non-3-enoxy]-8-oxo-octanoyl]oxy-2-[[8-[(Z)-non-3-enoxy]-8-oxo-octanoyl]oxymethyl]propyl]O1-[(Z)-non-3-enyl]octanedioate (0.3 g, 271.61 mol, 13.27% yield) was obtained as a colorless oil. ¹H NMR (400 MHz, CDCl₃) δ ppm 5.55-5.47 (m, 2H), 5.42-5.25 (m, 2H), 4.75 (s, 1H), 4.22-4.02 (m, 12H), 3.39 (s, 2H), 3.00-2.61 (m, 6H), 2.42-2.27 (m, 16H), 2.08-2.01 (m, 4H), 1.88 (s, 4H), 1.82-1.71 (m, 4H), 1.69-1.57 (m, 12H), 1.54-1.43 (m, 2H), 1.38-1.26 (m, 28H), 0.96-0.79 (m, 12H).

### Example 68—Compound 61

### O6-[2-[[2-(1-adamantyl)acetyl]oxymethyl]-2-[[6-[(Z)-non-3-enoxy]-6-oxo-hexanoyl]-oxymethyl]-3-[4-[2-(2-pyrrolidin-1-ylethylcarbamoyloxy)ethyl]octanoyloxy]propyl]O1-[(Z)-non-3-enyl]hexanedioate

### Synthetic Scheme:

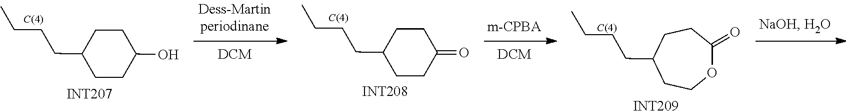

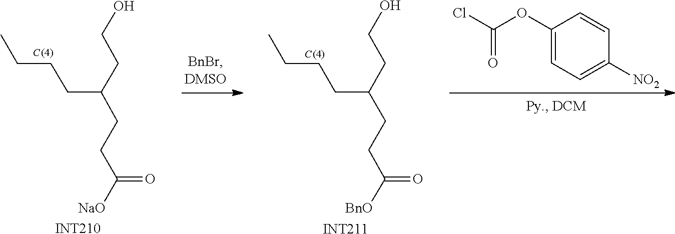

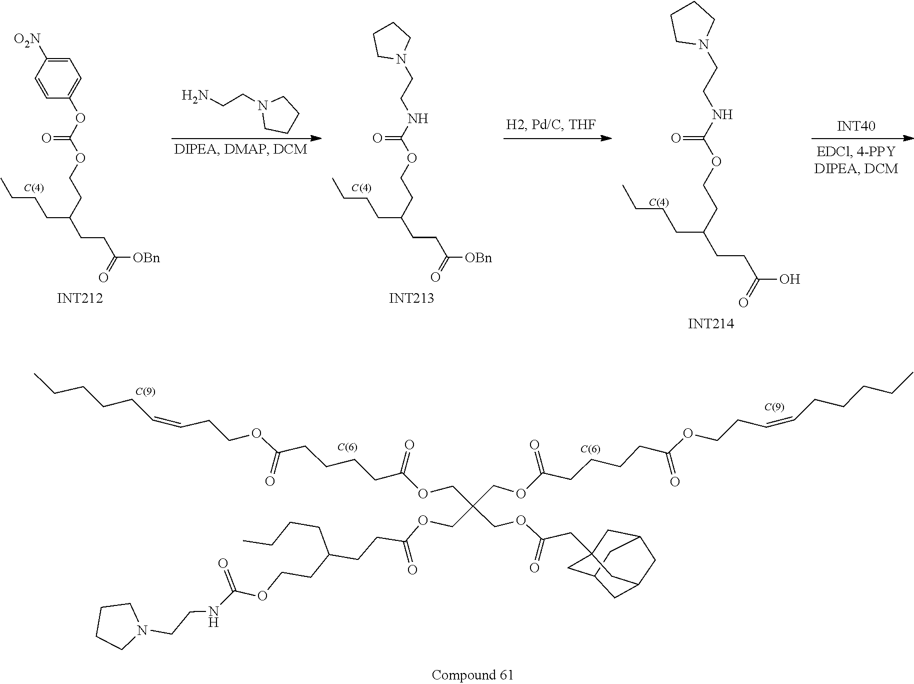

### Step 1:

To a solution of 4-butylcyclohexanol (10 g, 64.00 mmol, 1 eq) in DCM (100 mL) was added Dess-Martin (28.5 g, 67.20 mmol, 20.82 mL, 1.1 eq) at 0° C. under N₂ atmosphere. The reaction mixture was stirred at 25° C. for 12 h under N₂ atmosphere. The reaction mixture was concentrated under reduced pressure to get the crude product. The crude product was purified by silica gel column chromatography (ethyl acetate in petroleum ether, from 0/1 to 1/30) to give the desired product 4-butylcyclohexanone (9 g, 58.35 mmol, 91.18% yield) as colorless oil. ¹H NMR (400 MHz, CDCl₃) δ ppm 2.44-2.23 (m, 4H), 2.11-1.99 (m, 2H), 1.76-1.64 (m, 1H), 1.45-1.24 (m, 8H), 0.96-0.84 (m, 3H).

### Step 2:

To a solution of 4-butylcyclohexanone (9 g, 58.32 mmol, 1 eq) in DCM (90 mL) was added m-CPBA (17.4 g, 81.66 mmol, 80% purity, 1.5 eq) at 0° C. under N₂ atmosphere. The reaction mixture was stirred at 25° C. for 2 h under N₂ atmosphere. The suspension was filtered and the filter cake was washed with DCM (200 mL). The filtrate was diluted with sat. NaHCO₃ (200 mL) and extracted with DCM 1500 mL (500 mL*3). The combined organic layers were dried over Na₂SO₄, filtered and the filtrate was concentrated under reduced pressure to give a residue. The residue was purified by silica gel column chromatography (ethyl acetate in petroleum ether, from 5% to 40%) to give the desired product 5-butyloxepan-2-one (8 g, 46.99 mmol, 80.54% yield) as colorless oil.

### Step 3:

To a solution of 5-butyloxepan-2-one (8 g, 47 mmol, 1 eq) in H₂O (90 mL) was added NaOH (1.94 g, 49.32 mmol, 1.05 eq) under N₂ atmosphere. The reaction mixture was stirred at 100° C. for 12 h under N₂ atmosphere. The reaction mixture was extracted with EtOAc (100 mL*3) and the aqueous phase was lyophilized to get 4-(2-hydroxyethyl) octanoyloxysodium (9.6 g, 45.66 mmol, 97.17% yield).

### Step 4:

To a solution of 4-(2-hydroxyethyl)octanoyloxysodium (9.6 g, 45.68 mmol, 1 eq) in DMSO (96 mL) was added BnBr (7.8 g, 45.68 mmol, 5.44 mL, 1 eq) dropwise at 25° C. under N₂ atmosphere. The reaction mixture was stirred at 25° C. for 0.5 h under N₂ atmosphere. The reaction mixture was diluted with H₂O (500 mL) and extracted with EtOAc (200 mL×3). The combined organic layers were dried over Na₂SO₄, filtered and the filtrate was concentrated under reduced pressure to give a residue. The residue was purified by silica gel column chromatography (ethyl acetate in petroleum ether, from 70% to 90%) to give the desired product benzyl 4-(2-hydroxyethyl)octanoate (10 g, 35.10 mmol, 76.87% yield, 97.71% purity) as colorless oil. ¹H NMR (400 MHz, CDCl₃) δ ppm 7.45-7.29 (m, 5H), 5.12 (s, 2H), 3.74-3.60 (m, 2H), 2.38 (t, J=7.6 Hz, 2H), 1.73-1.44 (m, 7H), 1.36-1.26 (m, 4H), 0.89 (t, J=6.8 Hz, 3H).

### Step 5:

To a solution of benzyl 4-(2-hydroxyethyl)octanoate (10 g, 35.90 mmol, 1 eq) in DCM (100 mL) was added (4-nitrophenyl) carbonochloridate (14.50 g, 71.85 mmol, 2 eq) at 0° C. under N₂ atmosphere. Then Py. (5.7 g, 71.85 mmol, 5.80 mL, 2 eq) was added to the above reaction mixture drop wise at 0° C. under N₂ atmosphere. The reaction mixture was stirred at 25° C. for 1 h under N₂ atmosphere. The reaction mixture was concentrated under reduced pressure to give a residue. The residue was purified by silica gel column chromatography (ethyl acetate in petroleum ether, from 10% to 50%) to give the desired product benzyl 4-[2-(4-nitrophenoxy)carbonyloxyethyl]octanoate (12 g, 26.18 mmol, 72.89% yield, 96.76% purity). ¹H NMR (400 MHz, CDCl₃) δ ppm 8.38-8.19 (m, 2H), 7.49-7.28 (m, 7H), 5.13 (s, 2H), 4.38-4.28 (m, 2H), 2.45-2.35 (m, 2H), 1.77-1.66 (m, 4H), 1.63-1.35 (m, 1H), 1.34-1.26 (m, 6H), 0.94-0.87 (m, 3H).

### Step 6:

To a solution of benzyl 4-[2-(4-nitrophenoxy)carbonyloxyethyl]octanoate (12 g, 27.06 mmol, 1 eq) and 2-pyrrolidin-1-ylethanamine (6.18 g, 54.12 mmol, 2 eq) in DCM (120 mL) was added DIPEA (10.5 g, 81.18 mmol, 14.16 mL, 3 eq) and DMAP (0.33 g, 2.71 mmol, 0.1 eq) at 0° C. under N₂ atmosphere. The reaction mixture was stirred at 25° C. for 12 h under N₂ atmosphere. The reaction mixture was diluted with H₂O (500 mL) and extracted with EtOAc (200 mL×3). The combined organic layers were dried over Na₂SO₄, filtered and the filtrate was concentrated under reduced pressure to give a residue. The residue was purified by silica gel column chromatography (ethyl acetate in petroleum ether, from 45% to 60%) to give the desired product benzyl 4-[2-(2-pyrrolidin-1-ylethylcarbamoyloxy)ethyl]octanoate (9 g, 21.45 mmol, 79.29% yield, 99.78% purity) as yellow oil. ¹H NMR (400 MHz, CDCl₃) δ ppm 7.46-7.22 (m, 5H), 5.12 (s, 2H), 4.12-3.99 (m, 2H), 3.29 (d, J=5.6 Hz, 2H), 2.59 (t, J=6.0 Hz, 2H), 2.52 (s, 4H), 2.38-2.34 (m, 2H), 1.79-1.75 (m, 4H), 1.71-1.62 (m, 2H), 1.61-1.52 (m, 2H), 1.50-1.40 (m, 1H), 1.30-1.22 (m, 6H), 0.96-0.84 (m, 3H).

### Step 7:

To a solution of benzyl 4-[2-(2-pyrrolidin-1-ylethylcarbamoyloxy)ethyl]octanoate (9 g, 21.50 mmol, 1 eq) in THF (180 mL) was added Pd/C (2.00 g, 1.88 mmol, 10% purity, 0.1 eq) under Ar atmosphere. The reaction mixture was stirred at 25° C. for 12 h under H₂ (15 Psi) atmosphere. The suspension was filtered and the filtrate was concentrated under reduced pressure to give a residue. The residue was purified by silica gel column chromatography (methanol in dichloromethane, from 9% to 10%) to give the desired product 4-[2-(2 -pyrrolidin-1-ylethylcarbamoyloxy)ethyl]octanoic acid (4 g, 12.03 mmol, 55.94% yield, 98.76% purity).

### Step 8:

To a solution of O6-[2-[[2-(1-adamantyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (1 g, 1.22 mmol, 1 eq) in DCM (18 mL) was added EDCI (281.53 mg, 1.47 mmol, 1.2 eq), 4-[2-(2-pyrrolidin-1-ylethylcarbamoyloxy)ethyl]octanoic acid (803.93 mg, 2.45 mmol, 2 eq), DIPEA (395.43 mg, 3.06 mmol, 532.93 L, 2.5 eq) and 4-pyrrolidin-1-ylpyridine (18.14 mg, 122.38 mol, 0.1 eq) under N₂ atmosphere. The reaction mixture was stirred at 25° C. for 12 hr under N₂ atmosphere. The reaction mixture was concentrated under reduced pressure to remove solvent. The residue was diluted with H₂O (10 mL) and extracted with DCM 30 mL (10 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound 06-[2-[[2-(1-adamantyl)acetyl]oxymethyl]-2-[[6-[(Z)-non-3-enoxy]-6-oxo-hexanoyl]-oxymethyl]-3-[4-[2-(2-pyrrolidin-1-ylethylcarbamoyloxy)ethyl]octanoyloxy]propyl]O1-[(Z)-non-3-enyl]hexanedioate (0.13 g, 115.30 mol, 9.42% yield) was obtained as a colorless oil. ¹H NMR (400 MHz, CDCl₃) δ ppm 5.57-5.43 (m, 2H), 5.38-5.30 (m, 2H), 4.13-4.02 (m, 14H), 3.31 (d, J=5.4 Hz, 2H), 2.71-2.49 (m, 6H), 2.40-2.29 (m, 14H), 2.09-2.01 (m, 6H), 1.97 (s, 3H), 1.79 (s, 4H), 1.73-1.57 (m, 24H), 1.45-1.21 (m, 20H), 0.89 (t, J=6.8 Hz, 9H).

### Example 69—Compound 62

### O6-[2-[[2-(1-adamantyl)acetyl]oxymethyl]-2-[[6-[(Z)-non-3-enoxy]-6-oxo-hexanoyl]oxymethyl]-3-[4-(quinuclidine-4-carbonyloxy)decanoyloxy]propyl]O1-[(Z)-non-3-enyl]hexanedioate

### Synthetic Scheme:

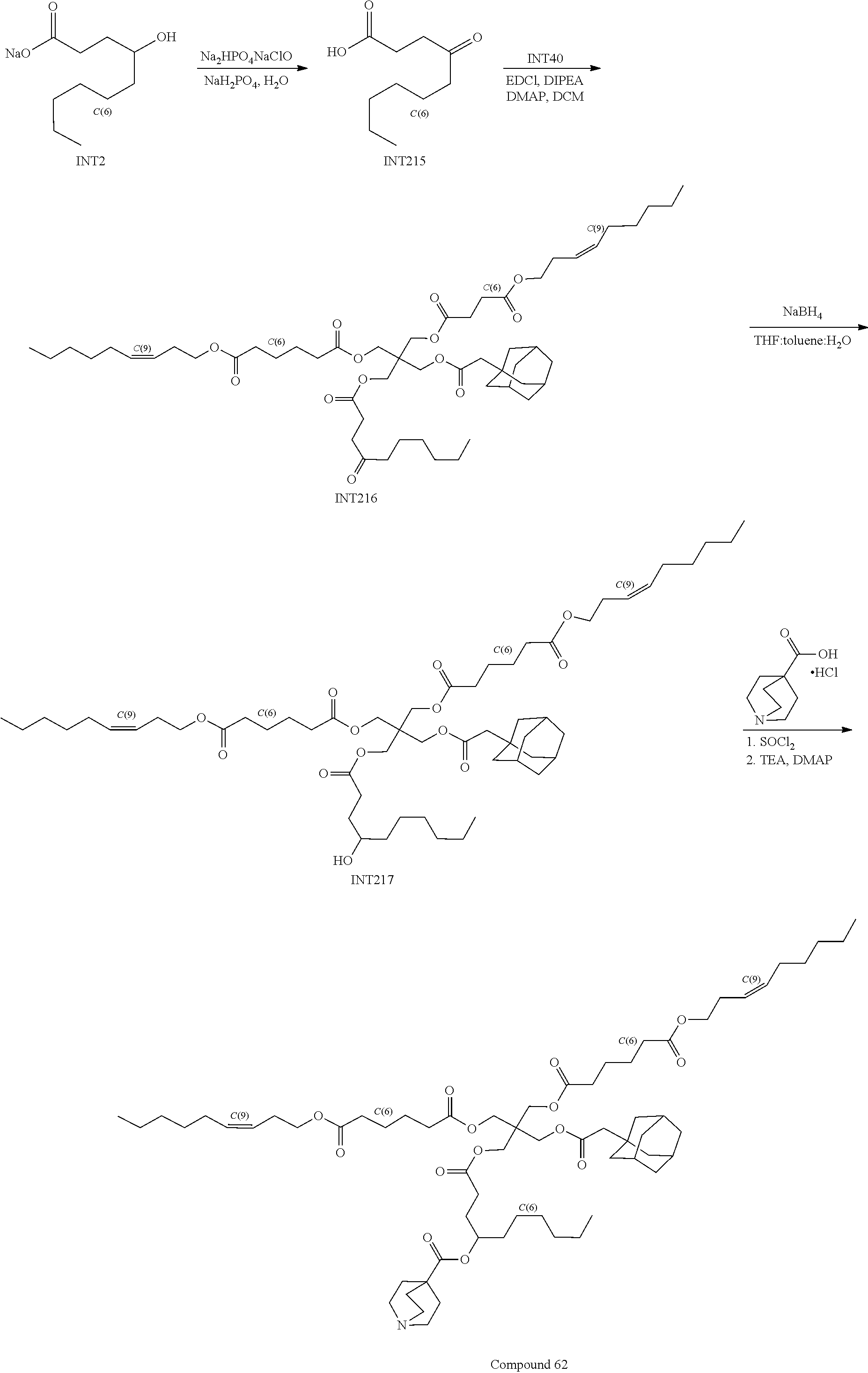

### Step 1:

To a solution of 4-hydroxydecanoyloxysodium (8 g, 38.05 mmol, 1 eq) in H₂O (40 mL) was added Na₂HPO₄ (4.8 g, 33.81 mmol, 4.80 mL, 1 eq), NaH₂PO₄ (1 g, 8.33 mmol, 1 eq) and NaClO (67.98 g, 91.32 mmol, 9.13 mL, 10% purity, 2.4 eq) at 20° C. under N₂ atmosphere. The reaction mixture was stirred at 20° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by silica gel column chromatography (petroleum ether/ethyl acetate, from 8/1 to 6/1) to give the desired product 4-oxodecanoic acid (4.5 g, 24.09 mmol, 63.31% yield, 99.7% purity) as a white solid. ¹H NMR (400 MHz, CDCl₃) δ ppm 2.75-2.69 (m, 2H), 2.66-2.60 (m, 2H), 2.45 (t, J=7.2 Hz, 2H), 1.66-1.51 (m, 2H), 1.36-1.24 (m, 6H), 0.92-0.83 (m, 3H).

### Step 2:

To a mixture of O6-[2-[[2-(1-adamantyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (1.3 g, 1.59 mmol, 1 eq) and 4-oxodecanoic acid (297 mg, 1.59 mmol, 1 eq) in DCM (13 mL) was added DIPEA (617 mg, 4.77 mmol, 831.54 L, 3 eq), EDCI (366 mg, 1.91 mmol, 1.2 eq) and DMAP (19 mg, 155.52 mol, 0.1 eq) at 20° C. under N₂ atmosphere. The reaction mixture was stirred at 20° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by silica gel column chromatography (petroleum ether/ethyl acetate, from 10/1 to 8/1) to give the desired product O6-[2-[[2-(1-adamantyl)acetyl]oxymethyl]-2-[[6-[(Z)-non-3-enoxy]-6-oxo-hexanoyl]oxy-methyl]-3-(4-oxodecanoyloxy)propyl]O1-[(Z)-non-3-enyl]hexanedioate (1.4 g, 1.41 mmol, 89.40% yield, 99.7% purity) as yellow oil. ¹H NMR (400 MHz, CDCl₃) δ ppm 5.58-5.44 (m, 2H), 5.39-5.27 (m, 2H), 4.14-4.04 (m, 12H), 2.74-2.68 (m, 2H), 2.61-2.54 (m, 2H), 2.44 (t, J=7.6 Hz, 2H), 2.41-2.28 (m, 12H), 2.10-2.00 (m, 7H), 1.96 (s, 3H), 1.76-1.58 (m, 19H), 1.40-1.22 (m, 20H), 0.95-0.81 (m, 9H).

### Step 3:

To a solution of O6-[2-[[2-(1-adamantyl)acetyl]oxymethyl]-2-[[6-[(Z)-non-3-enoxy]-6-oxo-hexanoyl]oxymethyl]-3-(4-oxodecanoyloxy) propyl]O1-[(Z)-non-3-enyl]hexanedioate (900 mg, 913.39 mol, 1 eq) in THF (4 mL), toluene (1 mL) and H₂O (2 mL) was added NaBH₄ (242.00 mg, 6.39 mmol, 7 eq) portion wise at 5° C. under N₂ atmosphere. The reaction mixture was stirred at 5° C. for 10 h under N₂ atmosphere. The reaction mixture was quenched by adding HCl (1M, 10 mL) at 5° C. under N₂ atmosphere. The reaction mixture was diluted with H₂O (100 mL) and extracted with DCM 100 mL (50 mL*2). The combined organic layers were dried over Na₂SO₄, filtered and the filtrate was concentrated under reduced pressure to give a residue. The residue was purified by silica gel column chromatography (petroleum ether/ethyl acetate, from 8/1 to 7/1) to give the desired product O6-[2-[[2-(1-adamantyl)acetyl]oxymethyl]-2-(4-hydroxydecanoyloxymethyl)-3-[6-[(Z)-non-3-enoxy]-6-oxohexanoyl]oxy-propyl]O1-[(Z)-non-3-enyl]hexanedioate (500 mg, 506.41 mol, 63.03% yield) as colorless oil. ¹H NMR (400 MHz, CDCl₃) δ ppm 5.58-5.45 (m, 2H), 5.41-5.26 (m, 2H), 4.15-4.04 (m, 12H), 3.65-3.55 (m, 1H), 2.47 (t, J=7.2 Hz, 2H), 2.41-2.30 (m, 12H), 2.10-2.02 (m, 6H), 1.97 (s, 3H), 1.75-1.59 (m, 22H), 1.41-1.21 (m, 22H), 0.90 (t, J=6.8 Hz, 9H).

### Step 4:

A suspension of quinuclidine-4-carboxylic acid (700 mg, 3.65 mmol, 1 eq, HCl) in SOCl₂ (6.55 g, 55.07 mmol, 4 mL, 13.19 eq) was heated to 80° C. and stirred for 5 h under N₂ atmosphere. The reaction mixture was concentrated under reduced pressure to get the crude product quinuclidine-4-carbonyl chloride (500 mg). Then a solution of O6-[2-[[2-(1-adamantyl)acetyl]oxymethyl]-2-(4-hydroxydecanoyloxymethyl)-3-[6-[(Z)-non-3-enoxy]-6-oxo-hexanoyl]-oxy-propyl]O1-[(Z)-non-3-enyl]hexanedioate (500 mg, 506.41 mol, 1 eq) in DCM (5 mL) was added TEA (153 mg, 1.51 mmol, 21.27 L, 3 eq) and DMAP (6 mg, 52.62 mol, 0.1 eq) under N₂ atmosphere. The reaction mixture was cooled to 0° C. under N₂ atmosphere. Then quinuclidine-4-carbonyl chloride (500 mg) in DCM (7 mL) was added to the above reaction mixture dropwise at 0° C. under N₂ atmosphere. The reaction mixture was stirred at 20° C. for 12 h under N₂ atmosphere. The reaction mixture was quenched with H₂O (50 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[2-(1-adamantyl)acetyl]oxymethyl]-2-[[6-[(Z)-non-3-enoxy]-6-oxo-hexanoyl]oxymethyl]-3-[4-(quinuclidine-4-carbonyloxy)decanoyloxy]propyl]O1-[(Z)-non-3-enyl]hexanedioate (100 mg, 100% purity) was obtained as colorless oil. ¹H NMR (400 MHz, CDCl₃) δ ppm 5.59-5.46 (m, 2H), 5.39-5.22 (m, 2H), 5.01-4.85 (m, 1H), 4.18-4.01 (m, 12H), 3.24-3.19 (m, 4H), 2.45-2.17 (m, 15H), 2.12-1.88 (m, 16H), 1.85-1.52 (m, 24H), 1.41-1.24 (m, 20H), 0.95-0.82 (m, 9H).

### Example 70—Compound 63

### O6-[2-(adamantane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

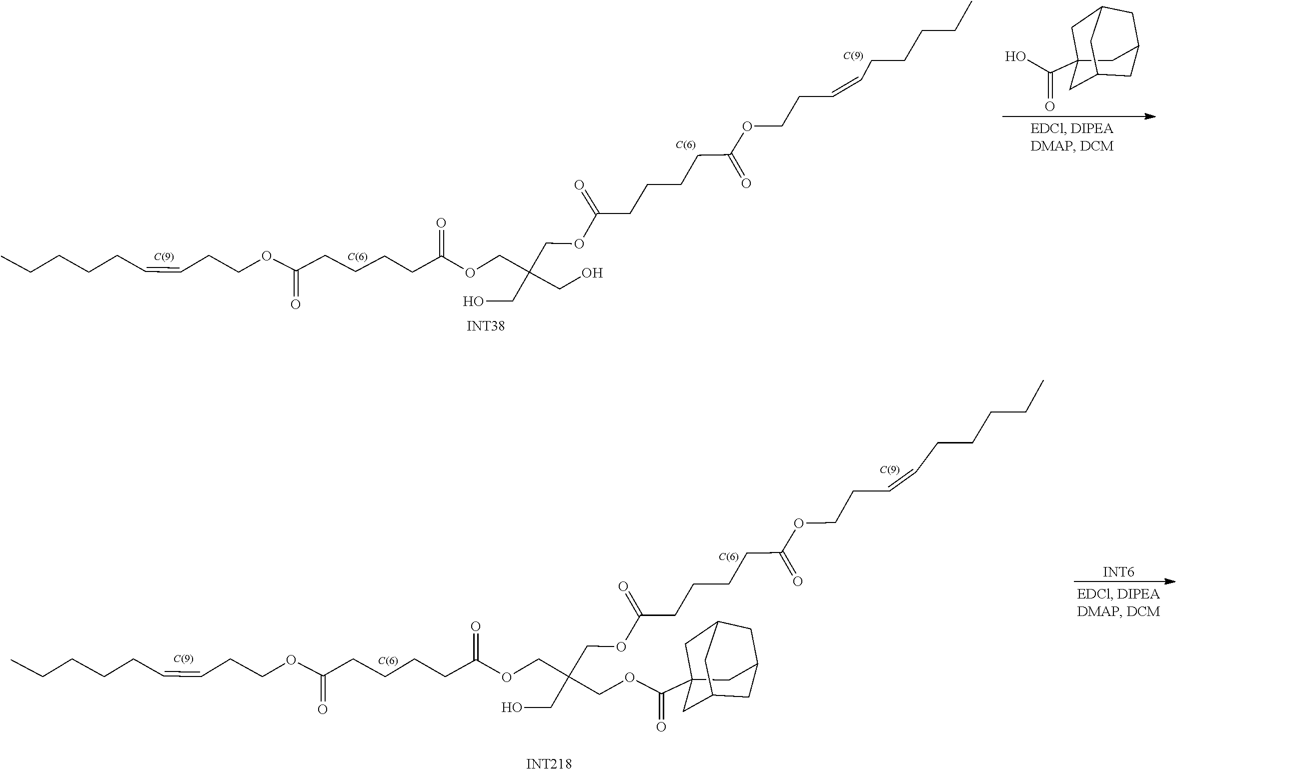

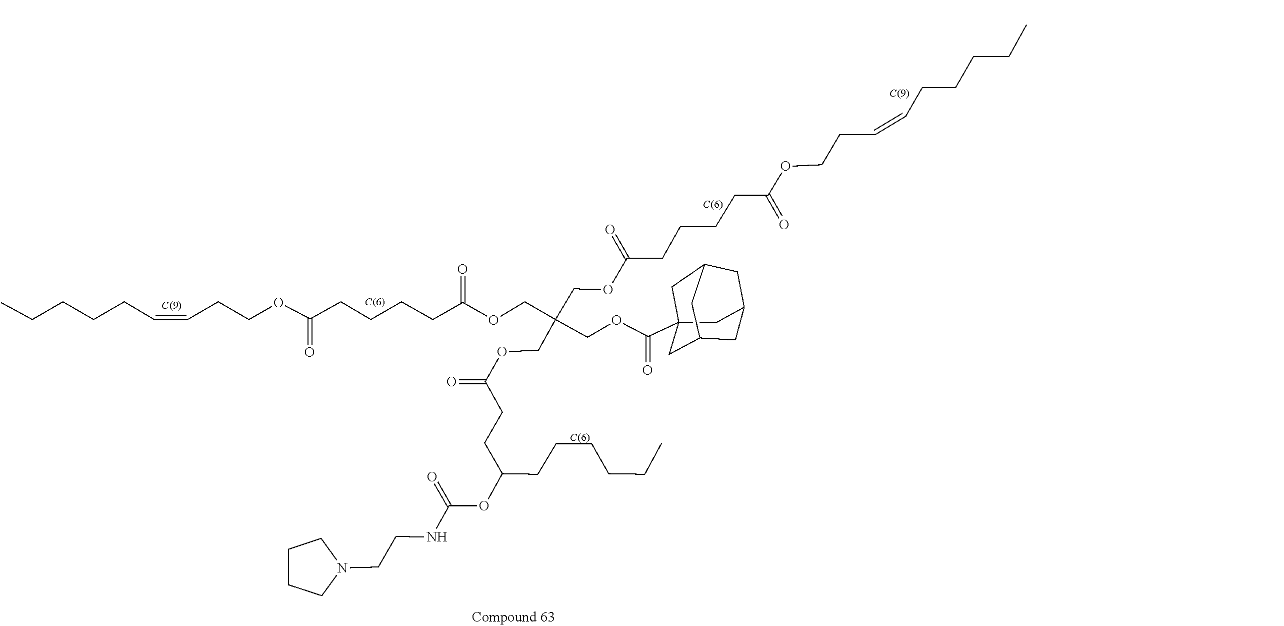 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CC3CC(CC(C3)C1)C2]

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (3 g, 4.68 mmol, 1 eq) in DCM (35 mL) was added EDCI (1.08 g, 5.62 mmol, 1.2 eq), adamantane-1-carboxylic acid (506.27 mg, 2.81 mmol, 0.6 eq), DMAP (57.19 mg, 468.13 mol, 0.1 eq) and DIPEA (1.51 g, 11.70 mmol, 2.04 mL, 2.5 eq) under N₂ atmosphere. The reaction mixture was stirred at 25° C. for 12 hr under N₂ atmosphere. The reaction mixture was diluted with H₂O (300 mL) and extracted with DCM 600 mL (300 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound O6-[2-(adamantane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1.5 g, 1.87 mmol, 39.90% yield) was obtained as a colorless oil. ¹H NMR (400 MHz, CDCl₃) δ ppm 5.56-5.29 (m, 4H), 4.13 (s, 4H), 4.10-4.05 (m, 6H), 3.51 (s, 2H), 2.41-2.31 (m, 12H), 2.07-2.02 (m, 6H), 1.88 (d, J=2.4 Hz, 6H), 1.78-1.62 (m, 16H), 1.38-1.25 (m, 12H), 0.90 (t, J=6.8 Hz, 6H).

### Step 2:

To a solution of 4-(2-pyrrolidin-1-ylethylcarbamoyloxy)decanoic acid (797.53 mg, 2.43 mmol, 1.5 eq) in DCM (20 mL) was added DIPEA (523.04 mg, 4.05 mmol, 704.91 μL, 2.5 eq), EDCI (372.39 mg, 1.94 mmol, 1.2 eq), DMAP (19.78 mg, 161.88 umol, 0.1 eq) and O6-[2-(adamantane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1.3 g, 1.62 mmol, 1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(adamantane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.12 g, 107.77 mol, 6.66% yield) was obtained as a colorless oil. ¹H NMR (400 MHz, CDCl₃) δ ppm 5.54-5.30 (m, 4H), 4.79-4.70 (m, 1H), 4.17-4.11 (m, 6H), 4.10-4.05 (m, 6H), 3.46-3.38 (m, 1H), 2.89-2.49 (m, 4H), 2.41-2.31 (m, 14H), 2.10-1.99 (m, 8H), 1.97-1.84 (m, 10H), 1.82-1.50 (m, 22H), 1.40-1.26 (m, 20H), 0.93-0.85 (m, 9H).

### Example 71—Compound 64

### O6-[2-[3-(1-adamantyl)propanoyloxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

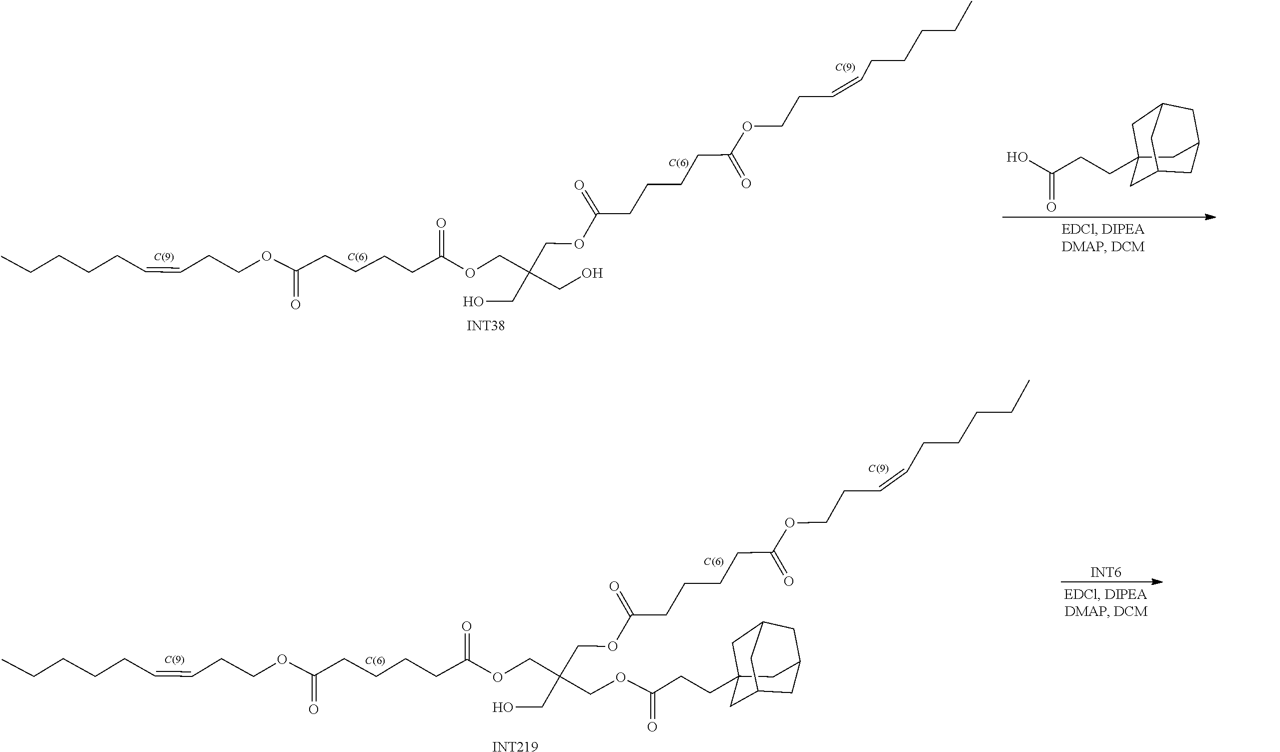

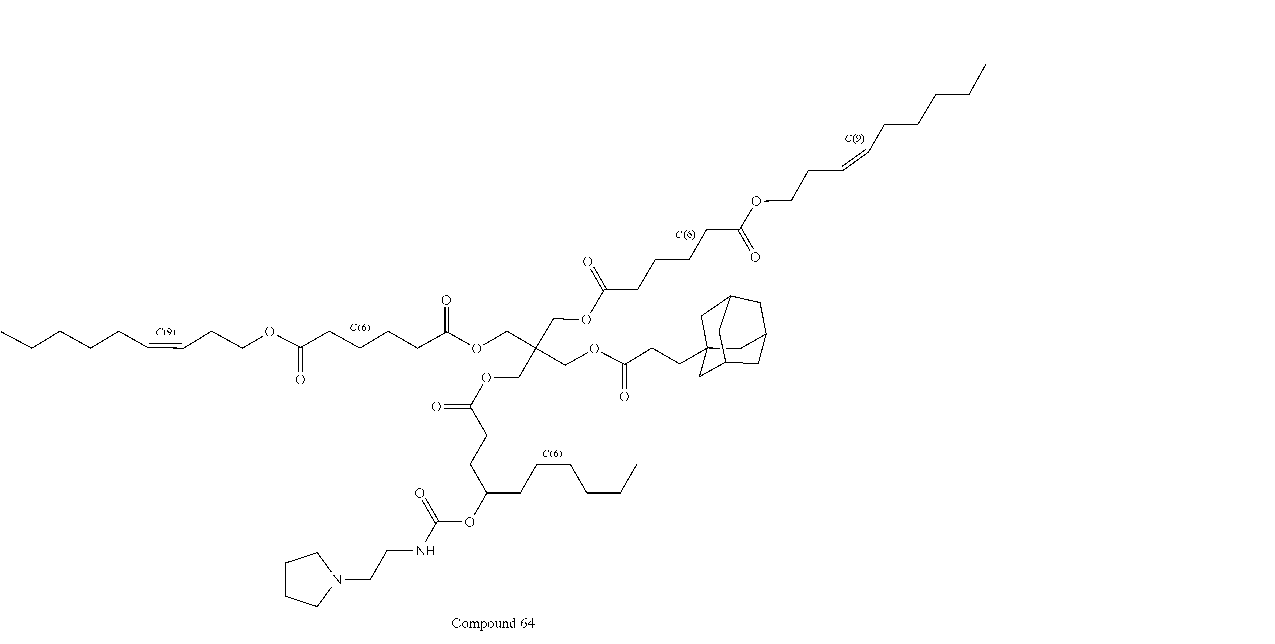

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (3 g, 4.68 mmol, 1 eq) in DCM (35 mL) was added EDCI (1.08 g, 5.62 mmol, 1.2 eq), DMAP (57.19 mg, 468.13 mol, 0.1 eq), 3-(1-adamantyl)propanoic acid (585.06 mg, 2.81 mmol, 0.6 eq) and DIPEA (1.51 g, 11.70 mmol, 2.04 mL, 2.5 eq) under N₂ atmosphere. The reaction mixture was stirred at 25° C. for 2 hr under N₂ atmosphere. The reaction mixture was diluted with H₂O (300 mL) and extracted with DCM 500 mL (250 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[3-(1-adamantyl)propanoyloxymethyl]-2-(hydroxymethyl)-3-[6-[(Z)-non-3-enoxy]-6-oxo-hexanoyl]oxy-propyl]O1-[(Z)-non-3-enyl]hexanediolate (1.2 g, 1.44 mmol, 30.84% yield) was obtained as a colorless oil. ¹H NMR (400 MHz, CDCl₃) δ ppm 5.56-5.28 (m, 4H), 4.13-4.05 (m, 10H), 3.51 (s, 2H), 2.41-2.27 (m, 15H), 2.04 (m, 4H), 1.96 (s, 3H), 1.75-1.58 (m, 16H), 1.46-1.29 (m, 18H), 0.90 (t, J=6.7 Hz, 6H).

### Step 2:

To a solution of O6-[2-[3-(1-adamantyl)propanoyloxymethyl]-2-(hydroxymethyl)-3-[6-[(Z)-non-3-enoxy]-6-oxo-hexanoyl]oxy-propyl]O1-[(Z)-non-3-enyl]hexanedioate (1.2 g, 1.44 mmol, 1 eq) in DCM (12 mL) was added EDCI (332.14 mg, 1.73 mmol, 1.2 eq), DMAP (17.64 mg, 144.38 mol, 0.1 eq), 4-(2-pyrrolidin-1-ylethylcarbamoyloxy)decanoic acid (948.44 mg, 2.89 mmol, 2 eq) and DIPEA (466.51 mg, 3.61 mmol, 628.72 L, 2.5 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[3-(1-adamantyl)propanoyloxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.1 g, 87.60 mol, 6.07% yield) was obtained as a colorless oil. ¹H NMR (400 MHz, CDCl₃) δ ppm 5.55-5.29 (m, 4H), 4.82-4.67 (m, 1H), 4.23-3.97 (m, 12H), 3.47-3.24 (m, 2H), 3.06-2.42 (m, 6H), 2.42-2.22 (m, 16H), 2.04 (m, 4H), 1.98-1.75 (m, 9H), 1.74-1.52 (m, 16H), 1.46 (s, 6H), 1.42-1.26 (m, 22H), 0.89 (m, 9H).

### Example 72—Compound 65

### O6-[2-[[2-(1-adamantyl)acetyl]oxymethyl]-2-[[6-oxo-6-[rac-(Z)-non-3-enoxy]hexanoyl]oxymethyl]-3-[4-(2-pyrrolidin-1-ylethylcarbamoyloxy)octanoyloxy]propyl]O1-[rac-(Z)-non-3-enyl]hexanedioate

### Synthetic Scheme:

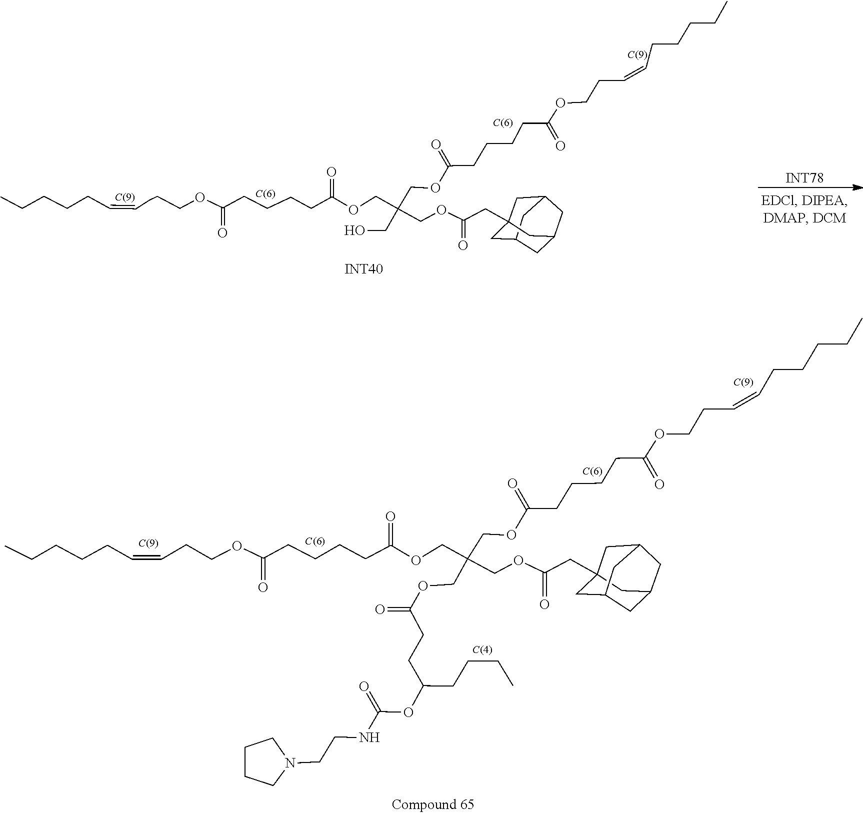

A mixture of O6-[2-[[2-(1-adamantyl)acetyl]oxymethyl]-2-(hydroxymethyl)-3-[6-oxo-6-[rac-(Z)-non-3-enoxy]hexanoyl]oxy-propyl]O1-[rac-(Z)-non-3-enyl]hexanedioate (1 g, 1.22 mmol, 1 eq), 4-(2-pyrrolidin-1-ylethylcarbamoyloxy)octanoic acid (441.16 mg, 1.47 mmol, 1.2 eq), DMAP (14.95 mg, 122.38 mol, 0.1 eq), EDCI (281.53 mg, 1.47 mmol, 1.2 eq) and DIPEA (395.43 mg, 3.06 mmol, 532.93 μL, 2.5 eq) in DCM (10 mL) was degassed and purged with N₂ for 3 times, and then the reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[2-(1-adamantyl)acetyl]oxymethyl]-2-[[6-oxo-6-[rac-(Z)-non-3-enoxy]hexanoyl]oxymethyl]-3-[4-(2-pyrrolidin-1-ylethylcarbamoyloxy)octanoyloxy]propyl]O1-[rac-(Z)-non-3-enyl]hexanedioate (0.1 g, 90.95 mol, 7.43% yield, 100% purity) was obtained as a colorless oil. ¹H NMR (400 MHz, CDCl₃) δ ppm 5.57-5.46 (m, 2H), 5.41-5.28 (m, 2H), 4.82-4.72 (m, 1H), 4.17-4.05 (m, 12H), 3.55-3.41 (m, 2H), 3.31-2.51 (m, 6H), 2.42-2.31 (m, 14H), 2.10-2.02 (m, 6H), 1.97 (s, 5H), 1.82-1.48 (m, 28H), 1.41-1.27 (m, 16H), 0.90 (t, J=6.8 Hz, 9H).

### Example 73—Compound 66

### O6-[2-[[2-(1-adamantyl)acetyl]oxymethyl]-2-[[6-[(Z)-non-3-enoxy]-6-oxohexanoyl]oxymethyl]-3-[4-(2-pyrrolidin-1-ylethylcarbamoyloxy)dodecanoyloxy]propyl]O1-[(Z)-non-3-enyl]hexanedioate

### Synthetic Scheme:

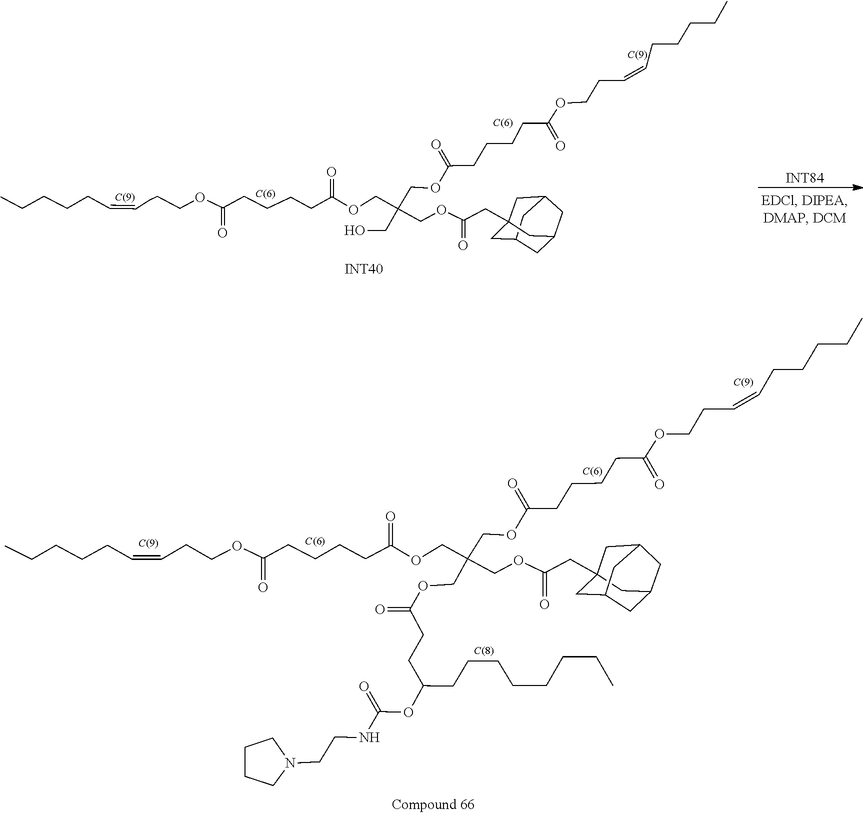

A mixture of O6-[2-[[2-(1-adamantyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (1 g, 1.22 mmol, 1 eq), 4-(2-pyrrolidin-1-ylethylcarbamoyloxy)dodecanoic acid (479.93 mg, 1.35 mmol, 1.1 eq), EDCI (281.53 mg, 1.47 mmol, 1.2 eq), DMAP (14.95 mg, 122.38 mol, 0.1 eq) and DIPEA (395.43 mg, 3.06 mmol, 532.93 μL, 2.5 eq) in DCM (10 mL) was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[2-(1-adamantyl)acetyl]oxymethyl]-2-[[6-[(Z)-non-3-enoxy]-6-oxohexanoyl]oxymethyl]-3-[4-(2-pyrrolidin-1-ylethylcarbamoyloxy)dodecanoyloxy]propyl]O1-[(Z)-non-3-enyl]hexanedioate (0.1 g, 86.54 μmol, 7.07% yield, 100% purity) was obtained as a colorless oil. ¹H NMR (400 MHz, CDCl₃) δ ppm 5.56-5.45 (m, 2H), 5.40-5.28 (m, 2H), 4.75 (s, 1H), 4.12 (s, 6H), 4.10-3.99 (m, 6H), 3.53-3.30 (m, 2H), 3.00-2.52 (m, 5H), 2.43-2.29 (m, 14H), 2.10-2.00 (m, 6H), 1.97 (s, 3H), 1.93-1.85 (m, 3H), 1.80 (d, J=7.1 Hz, 2H), 1.74-1.62 (m, 14H), 1.61-1.54 (m, 8H), 1.49 (s, 2H), 1.38-1.24 (m, 24H), 0.95-0.84 (m, 9H).

### Example 74—Compound 67

### O6-[2-(bicyclo[2.2.2]octane-1-carbonyloxymethyl)-2-[[6-[(Z)-non-3-enoxy]-6-oxo-hexanoyl]-oxymethyl]-3-[4-(2-pyrrolidin-1-ylethylcarbamoyloxy)decanoyloxy]propyl]O1-[(Z)-non-3-enyl]hexanedioate

### Synthetic Scheme:

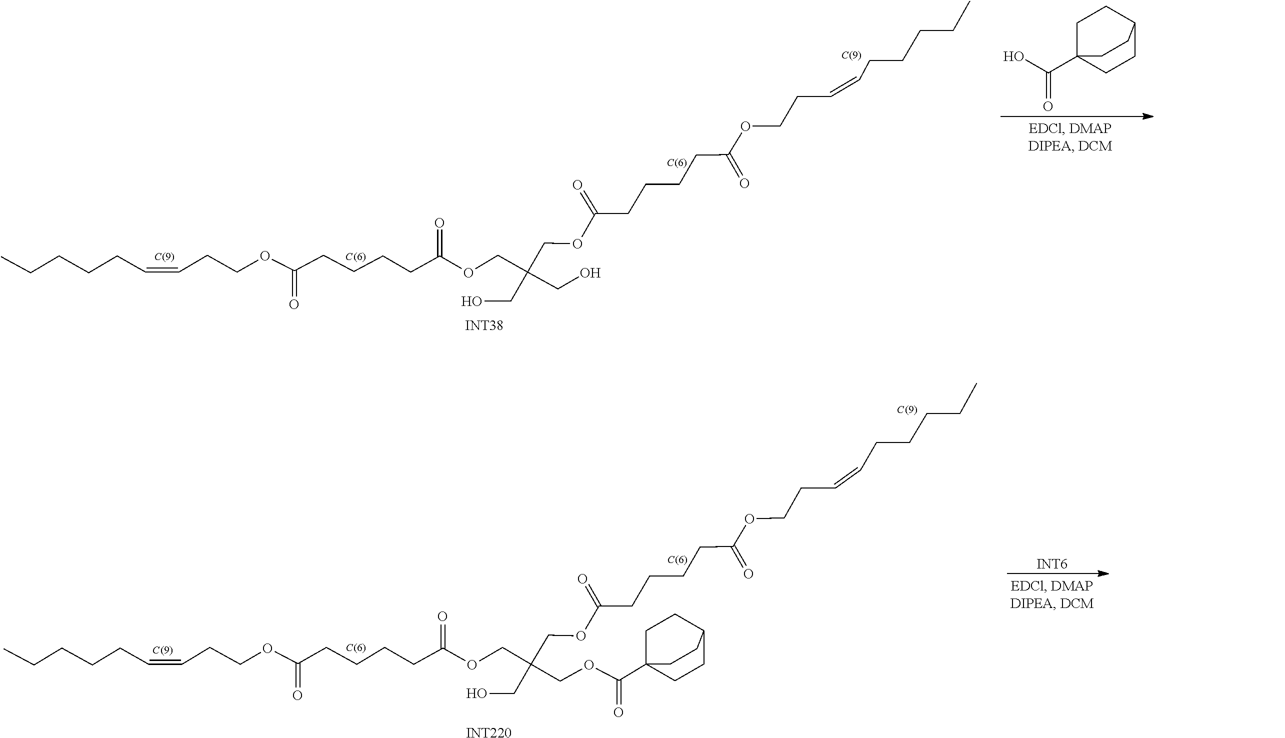

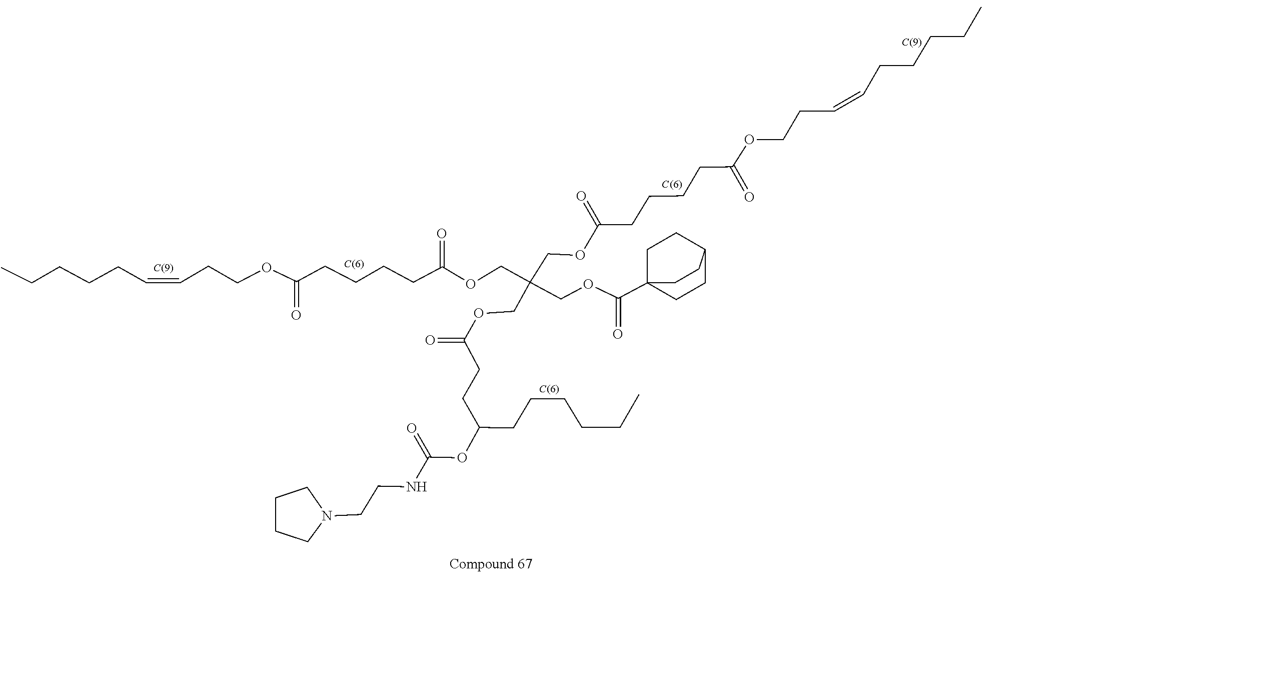 [SMILES: CCCCC/C=C\CCOC(=O)CCCCC(=O)OCC(COC(=O)CCCCC(=O)OCC/C=C\CCCCC)(COC(=O)CCC(CCCCCC)OC(=O)NCCN1CCCC1)COC(=O)C12CCC(CC1)CC2]

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (22 mL) was added EDCI (717.93 mg, 3.75 mmol, 1.2 eq), DMAP (38.13 mg, 312.09 mol, 0.1 eq), bicyclo[2.2.2]octane-1-carboxylic acid (288.76 mg, 1.87 mmol, 0.6 eq) and DIPEA (1.01 g, 7.80 mmol, 1.36 mL, 2.5 eq) under N₂ atmosphere. The reaction mixture was stirred at 25° C. for 10 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=I/O to 1/1). Compound O6-[2-(bicyclo[2.2.2]octane-1-carbonyloxy-methyl)-2-(hydroxymethyl)-3-[6-[(Z)-non-3-enoxy]-6-oxo-hexanoyl]oxy-propyl]O1-[(Z)-non-3-enyl]hexanedioate (0.8 g, 1.03 mmol, 32.99% yield) was obtained as a colorless oil. ¹H NMR (400 MHz, CDCl₃) δ ppm 5.55-5.30 (m, 4H), 4.14-4.05 (m, 10H), 3.49 (s, 2H), 2.63 (s, 1H), 2.41-2.31 (m, 12H), 2.08-2.00 (m, 4H), 1.75-1.57 (m, 22H), 1.40-1.27 (m, 12H), 0.90 (t, J=6.8 Hz, 6H).

### Step 2:

To a solution of O6-[2-(bicyclo[2.2.2]octane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.8 g, 1.03 mmol, 1 eq) in DCM (12 mL) was added EDCI (236.84 mg, 1.24 mmol, 1.2 eq), DMAP (12.58 mg, 102.96 mol, 0.1 eq), 4-(2-pyrrolidin-1-ylethylcarbamoyloxy)decanoic acid (405.78 mg, 1.24 mmol, 1.2 eq) and DIPEA (332.66 mg, 2.57 mmol, 448.32 μL, 2.5 eq) under N₂ atmosphere. The reaction mixture was stirred at 25° C. for 10 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: Phenomenex Luna C18 100*30 mm*3 um; mobile phase: [H₂O (0.04% HCl)-ACN:THF=1:1]; gradient: 30%-80% B over 12.0 min) to get compound O6-[2-(bicyclo[2.2.2]octane-1-carbonyloxymethyl)-2-[[6-[(Z)-non-3-enoxy]-6-oxo-hexanoyl]-oxymethyl]-3-[4-(2-pyrrolidin-1-ylethylcarbamoyloxy)decanoyloxy]propyl]O1-[(Z)-non-3-enyl]hexanedioate (0.15 g, 137.94 mol, 13.40% yield, 100% purity, HCl salt) as a colorless oil. ¹H NMR (400 MHz, CD₃OD) δ ppm 5.56-5.31 (m, 4H), 4.80-4.72 (m, 1H), 4.22-4.05 (m, 12H), 3.72 (d, J=4.8 Hz, 2H), 3.52-3.45 (m, 2H), 3.35-3.32 (m, 2H), 3.18-3.10 (m, 2H), 2.43-2.32 (m, 14H), 2.23-2.14 (m, 2H), 2.11-2.01 (m, 6H), 1.99-1.90 (m, 1H), 1.84-1.68 (m, 8H), 1.68-1.54 (m, 18H), 1.42-1.28 (m, 20H), 0.95-0.86 (m, 9H).

### Example 75—Compound 69

### O6-[2-[[6-[(Z)-non-3-enoxy]-6-oxo-hexanoyl]oxymethyl]-2-[4-(2-pyrrolidin-1-ylethylcarbamoyloxy)decanoyloxymethyl]-3-[3-(trifluoromethyl)bicyclo[1.1.1]pentane-1-carbonyl]oxy-propyl]O1-[(Z)-non-3-enyl]hexanedioate

### Synthetic Scheme:

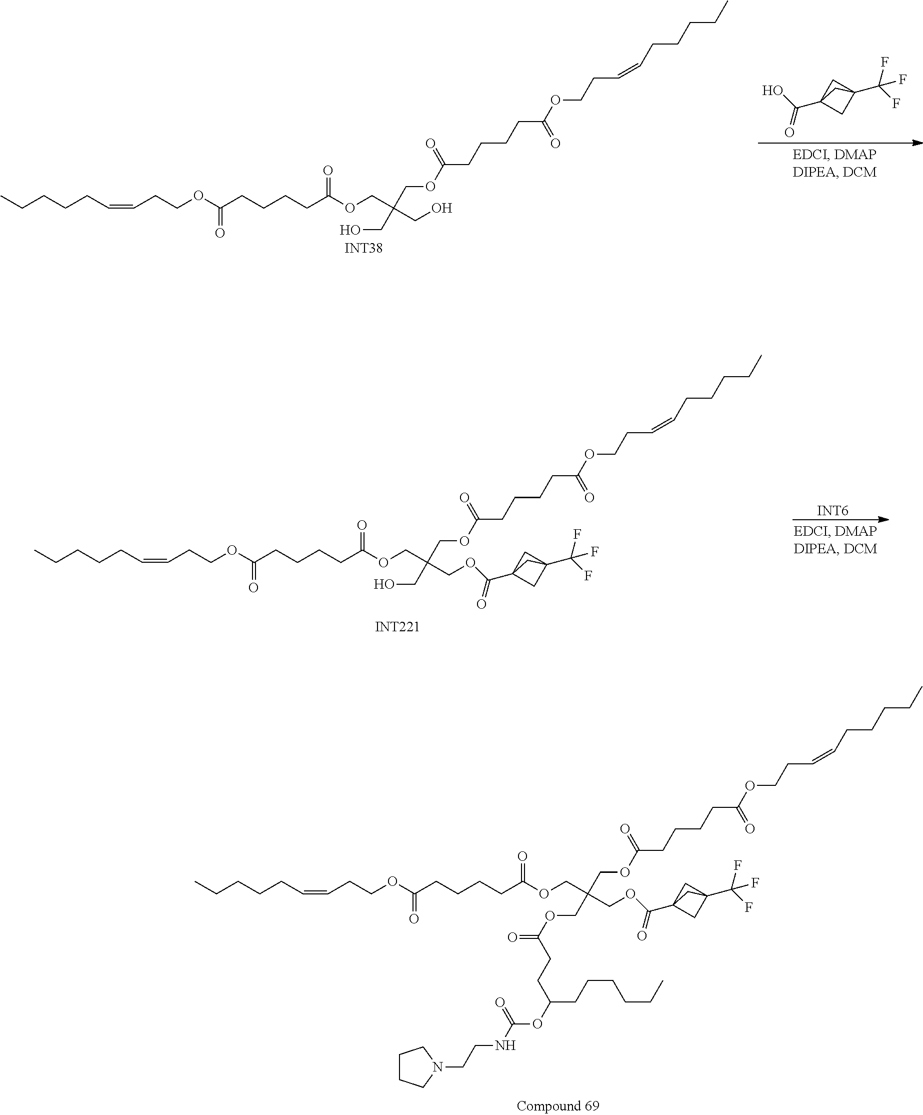

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 3 -(trifluoromethyl)bicyclo[1.1.1]pentane-1-carboxylic acid (674.58 mg, 3.75 mmol, 1.2 eq), DMAP (38.13 mg, 312.09 mol, 0.1 eq), EDCI (717.93 mg, 3.75 mmol, 1.2 eq) and DIPEA (1.01 g, 7.80 mmol, 1.36 mL, 2.5 eq) in DCM (20 mL) was stirred at 25° C. for 12 hours under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[[6-[(Z)-non-3-enoxy]-6-oxo-hexanoyl]oxymethyl]-3-[3-(trifluoromethyl)bicyclo[1.1.1]pentane-1-carbonyl]oxy-propyl]O1-[(Z)-non-3-enyl]hexanedioate (0.85 g, 1.06 mmol, 33.92% yield) was obtained as a colorless oil. ¹H NMR (400 MHz, CDCl₃) δ ppm 5.56-5.47 (m, 2H), 5.38-5.30 (m, 2H), 4.14-4.05 (m, 10H), 3.52 (s, 2H), 2.41-2.31 (m, 12H), 2.27 (s, 6H), 2.07-2.01 (m, 4H), 1.69-1.63 (m, 8H), 1.57 (d, J=3.0 Hz, 1H), 1.39-1.27 (m, 12H), 0.90 (t, J=6.8 Hz, 6H).

### Step 2:

A mixture of O6-[2-(hydroxymethyl)-2-[[6-[(Z)-non-3-enoxy]-6-oxo-hexanoyl]oxymethyl]-3-[3-(trifluoromethyl)bicyclo[1.1.1]pentane-1-carbonyl]oxy-propyl]O1-[(Z)-non-3-enyl]hexanedioate (0.85 g, 1.06 mmol, 1 eq), 4-(2-pyrrolidin-1-ylethylcarbamoyloxy)decanoic acid (417.23 mg, 1.27 mmol, 1.2 eq), DMAP (12.93 mg, 105.86 mol, 0.1 eq), EDCI (243.52 mg, 1.27 mmol, 1.2 eq) and DIPEA (342.04 mg, 2.65 mmol, 460.97 μL, 2.5 eq) in DCM (8.5 mL) was stirred at 25° C. for 12 hours under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: Phenomenex Luna C18 100*30 mm*3 um; mobile phase: [H₂O (0.04% HCl)-ACN:THF=1:1]; gradient: 40%-80% B over 12.0 min) to get compound O6-[2-[[6-[(Z)-non-3-enoxy]-6-oxo-hexanoyl]oxymethyl]-2-[4-(2-pyrrolidin-1-ylethylcarbamoyloxy)decanoyloxymethyl]-3-[3-(trifluoromethyl)bicyclo[1.1.1]pentane-1-carbonyl]oxy-propyl]O1-[(Z)-non-3-enyl]hexanedioate (0.15 g, 134.72 mol, 12.73% yield, 100% purity, HCl salt) as a colorless oil. ¹H NMR (400 MHz, CDCl₃) δ ppm 12.54-12.22 (m, 1H), 6.39 (s, 1H), 5.55-5.47 (m, 2H), 5.38-5.30 (m, 2H), 4.79-4.70 (m, 1H), 4.17-4.04 (m, 12H), 3.90-3.83 (m, 2H), 3.71-3.60 (m, 2H), 3.29-3.19 (m, 2H), 2.91-2.80 (m, 2H), 2.39-2.31 (m, 14H), 2.26 (s, 8H), 2.10-2.01 (m, 6H), 1.99-1.86 (m, 2H), 1.83-1.76 (m, 1H), 1.68-1.63 (m, 8H), 1.60-1.54 (m, 1H), 1.53-1.46 (m, 1H), 1.35-1.26 (m, 18H), 0.92-0.86 (m, 9H).

### Example 76—Compound 70

### O6-[2-(bicyclo[3.2.1]octane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

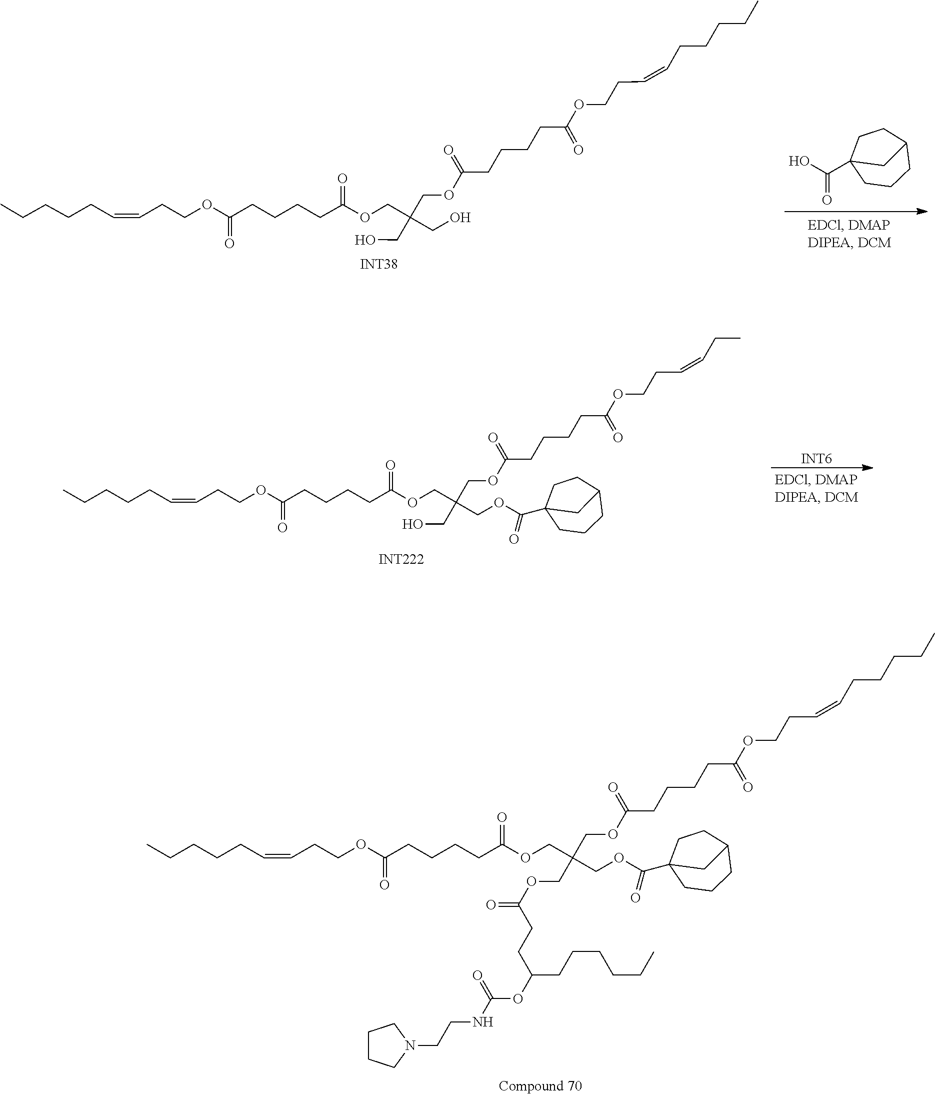

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), DMAP (28.60 mg, 234.07 mol, 0.1 eq) and bicyclo[3.2.1]octane-1-carboxylic acid (360.94 mg, 2.34 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[3.2.1]octane-1-carbonyloxymethyl)-2-(hydroxymethyl)-3-[6-[(Z)-non-3-enoxy]-6-oxo-hexanoyl]-oxy-propyl]O1-[(Z)-non-3-enyl]hexanedioate (1 g, 1.29 mmol, 54.98% yield) was obtained as a colorless oil.

### Step 2:

To a solution of O6-[2-(bicyclo[3.2.1]octane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.29 mmol, 1 eq) in DCM (10 mL) was added EDCI (296.05 mg, 1.54 mmol, 1.2 eq), DIPEA (415.82 mg, 3.22 mmol, 560.40 L, 2.5 eq), 4-(2-pyrrolidin-1-ylethylcarbamoyloxy)decanoic acid (422.69 mg, 1.29 mmol, 1 eq) and DMAP (15.72 mg, 128.69 mol, 0.1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 30%-75% B over 12.0 min) to get compound O6-[2-(bicyclo[3.2.1]octane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 137.94 mol, 10.72% yield, HCl salt) as a colorless oil. ¹H NMR (400 MHz, CDCl₃) δ ppm 12.36 (d, J=3.4 Hz, 1H), 6.31 (s, 1H), 5.60-5.44 (m, 2H), 5.38-5.28 (m, 2H), 4.82-4.60 (m, 1H), 4.19-4.05 (m, 12H), 3.86 (s, 2H), 3.72-3.59 (m, 2H), 3.25 (s, 2H), 2.96-2.76 (m, 2H), 2.43-2.28 (m, 16H), 2.17-2.01 (m, 6H), 1.91-1.79 (m, 4H), 1.75-1.49 (m, 20H), 1.43 (d, J=4.8 Hz, 1H), 1.39-1.26 (m, 20H), 0.94-0.83 (m, 9H).

### Example 77—Compound 71

### O6-[2-[[4-(difluoromethyl)bicycle[2.2.2]octane-1-carbonyl]-oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

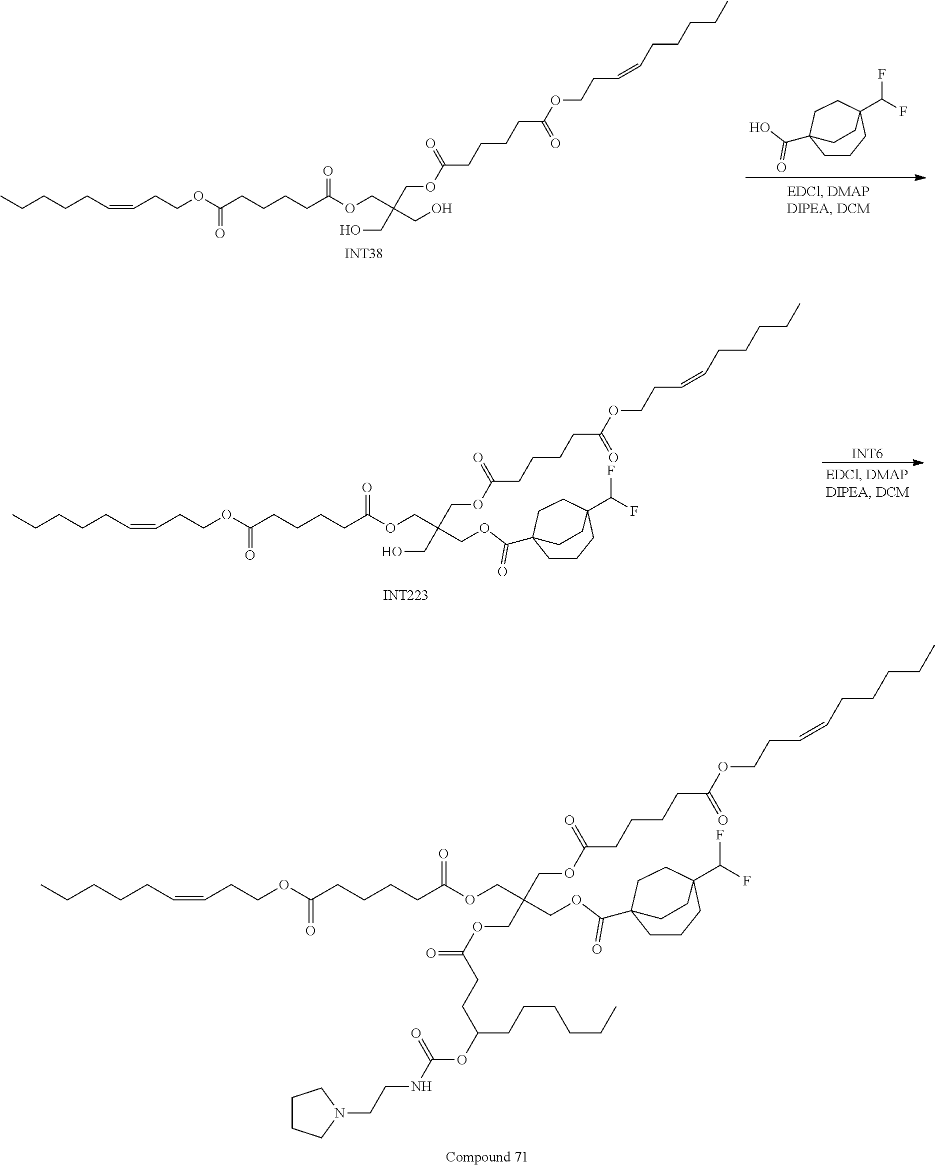

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), DMAP (28.60 mg, 234.07 mol, 0.1 eq) and 4-(difluoromethyl)bicyclo[2.2.2]octane-1-carboxylic acid (478.00 mg, 2.34 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[4-(difluoromethyl)bicyclo[2.2.2]octane-1-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 967.30 mol, 41.33% yield) was obtained as a colorless oil.

### Step 2:

To a solution of O6-[2-[[4-(difluoromethyl)bicyclo[2.2.2]octane-1-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 967.30 mol, 1 eq) in DCM (10 mL) was added EDCI (222.52 mg, 1.16 mmol, 1.2 eq), DIPEA (312.54 mg, 2.42 mmol, 421.22 μL, 2.5 eq), DMAP (11.82 mg, 96.73 mol, 0.1 eq) and 4-(2-pyrrolidin-1-ylethylcarbamoyloxy)decanoic acid (381.25 mg, 1.16 mmol, 1.2 eq) under N₂ atmosphere. The reaction mixture was stirred at 25° C. for 10 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 30%-75% B over 15.0 min) to get compound O6-[2-[[4-(difluoromethyl)bicycle[2.2.2]oct-ane-1-carbonyl]-oxymethyl]-2-[[6-[(Z)-non-3-enoxy]-6-oxo-hexa-noyl]oxymethyl]-3-[4-(2-pyrrolidin-1-ylethylcarbamoyloxy)decanoyloxy]propyl]O1-[(Z)-non-3-enyl]hexanedioate (0.15 g, 131.87 mol, 13.63% yield, HCl salt) as a colorless oil. ¹H NMR (400 MHz, CDCl₃) δ ppm 12.39 (s, 1H), 6.31 (t, J=5.8 Hz, 1H), 5.60-5.44 (m, 2H), 5.40-5.24 (m, 2H), 4.80-4.66 (m, 1H), 4.19-4.05 (m, 12H), 3.86 (s, 2H), 3.71-3.61 (m, 2H), 3.30-3.20 (m, 2H), 2.95-2.76 (m, 2H), 2.47-2.22 (m, 18H), 2.14-2.01 (m, 6H), 1.95-1.74 (m, 6H), 1.68-1.61 (m, 10H), 1.60-1.42 (m, 8H), 1.38-1.25 (m, 20H), 0.95-0.83 (m, 9H).

### Example 78—Compound 72

### O6-[2-(bicyclo[1.1.1]pentane-1-carbonyloxymethyl)-2-[[6-[(Z)-non-3-enoxy]-6-oxohexanoy-1]oxymethyl]-3-[4-(2-pyrrolidin-1-ylethylcarbamoyloxy)decanoyloxy]propyl]O1-[(Z)-non-3-enyl]hexanedioate

### Synthetic Scheme:

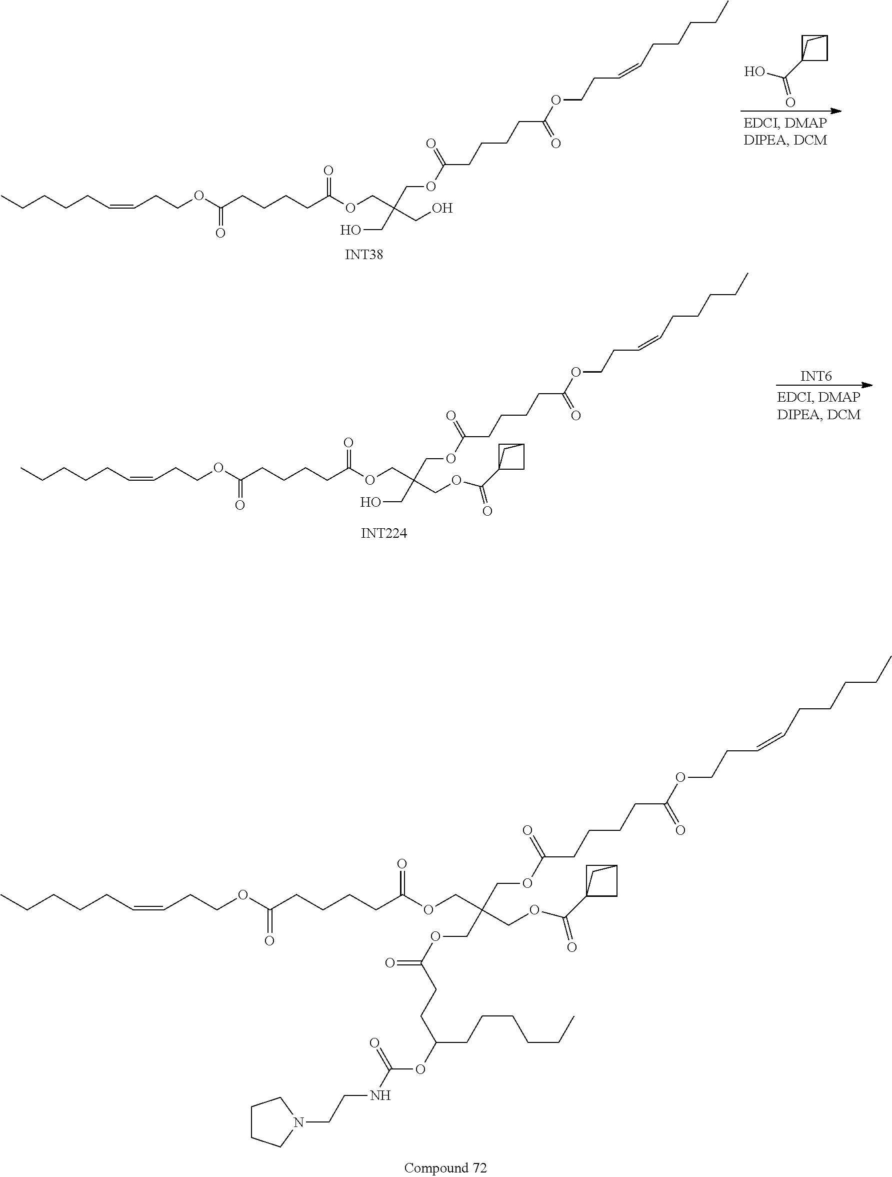

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.a12 mmol, 1 eq), bicyclo[1.1.1]pentane-1-carboxylic acid (209.96 mg, 1.87 mmol, 0.6 eq), DMAP (38.13 mg, 312.09 mol, 0.1 eq), DIPEA (1.01 g, 7.80 mmol, 1.36 mL, 2.5 eq) and EDCI (717.93 mg, 3.75 mmol, 1.2 eq) in DCM (22 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[1.1.1]pentane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.16 mmol, 37.06% yield) was obtained as a colorless oil. ¹H NMR (400 MHz, CDCl₃) δ ppm 5.56-5.47 (m, 2H), 5.38-5.29 (m, 2H), 4.12-4.05 (m, 10H), 3.51 (s, 2H), 2.45 (s, 1H), 2.40-2.31 (m, 12H), 2.09 (s, 6H), 2.07-2.01 (m, 4H), 1.66 (d, J=6.8 Hz, 8H), 1.39-1.27 (m, 12H), 0.90 (t, J=6.8 Hz, 6H).

### Step 2:

A mixture of O6-[2-(bicyclo[1.1.1]pentane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.16 mmol, 1 eq), 4-(2-pyrrolidin-1-ylethylcarbamoyloxy)decanoic acid (455.83 mg, 1.39 mmol, 1.2 eq), EDCI (266.05 mg, 1.39 mmol, 1.2 eq), DMAP (14.13 mg, 115.65 mol, 0.1 eq) and DIPEA (373.68 mg, 2.89 mmol, 503.62 L, 2.5 eq) in DCM (13 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 mm*5 um; mobile phase: [H₂O (0.04% HCl)-ACN:THF=1:1]; gradient: 40%-80% B over 12.0 min) to get compound O6-[2-(bicyclo[1.1.1]pentane-1-carbonyloxymethyl)-2-[[6-[(Z)-non-3-enoxy]-6-oxohexanoy-1]oxymethyl]-3-[4-(2-pyrrolidin-1-ylethylcarbamoyloxy)decanoyloxy]propyl]O1-[(Z)-non-3-enyl]hexanedioate (0.15 g, 143.49 mol, 12.41% yield, 100% purity, HCl salt) as a colorless oil. ¹H NMR (400 MHz, CDCl₃) δ ppm 12.43-11.78 (m, 1H), 6.32 (t, J=5.6 Hz, 1H), 5.55-5.47 (m, 2H), 5.38-5.30 (m, 2H), 4.80-4.69 (m, 1H), 4.16-4.03 (m, 12H), 3.90-3.81 (m, 2H), 3.69-3.61 (m, 2H), 3.30- 3.19 (m, 2H), 2.91-2.81 (m, 2H), 2.44 (s, 1H), 2.36 (d, J=7.4 Hz, 14H), 2.27-2.21 (m, 2H), 2.08 (s, 8H), 2.05-2.01 (m, 4H), 1.96-1.86 (m, 2H), 1.81-1.77 (m, 1H), 1.65 (d, J=2.6 Hz, 8H), 1.55-1.44 (m, 2H), 1.35-1.26 (m, 18H), 0.93-0.85 (m, 9H).

### Example 79—Compound 73

### O6-[2-[(2-cyclobutylacetyl)oxymethyl]-2-[[6-[(Z)-non-3-enoxy]-6-oxo-hexanoyl]oxymethyl]-3-[4-(2-pyrrolidin-1-ylethylcarbamoyloxy) decanoyloxy]propyl]O1-[(Z)-non-3-enyl]hexanedioate

### Synthetic Scheme:

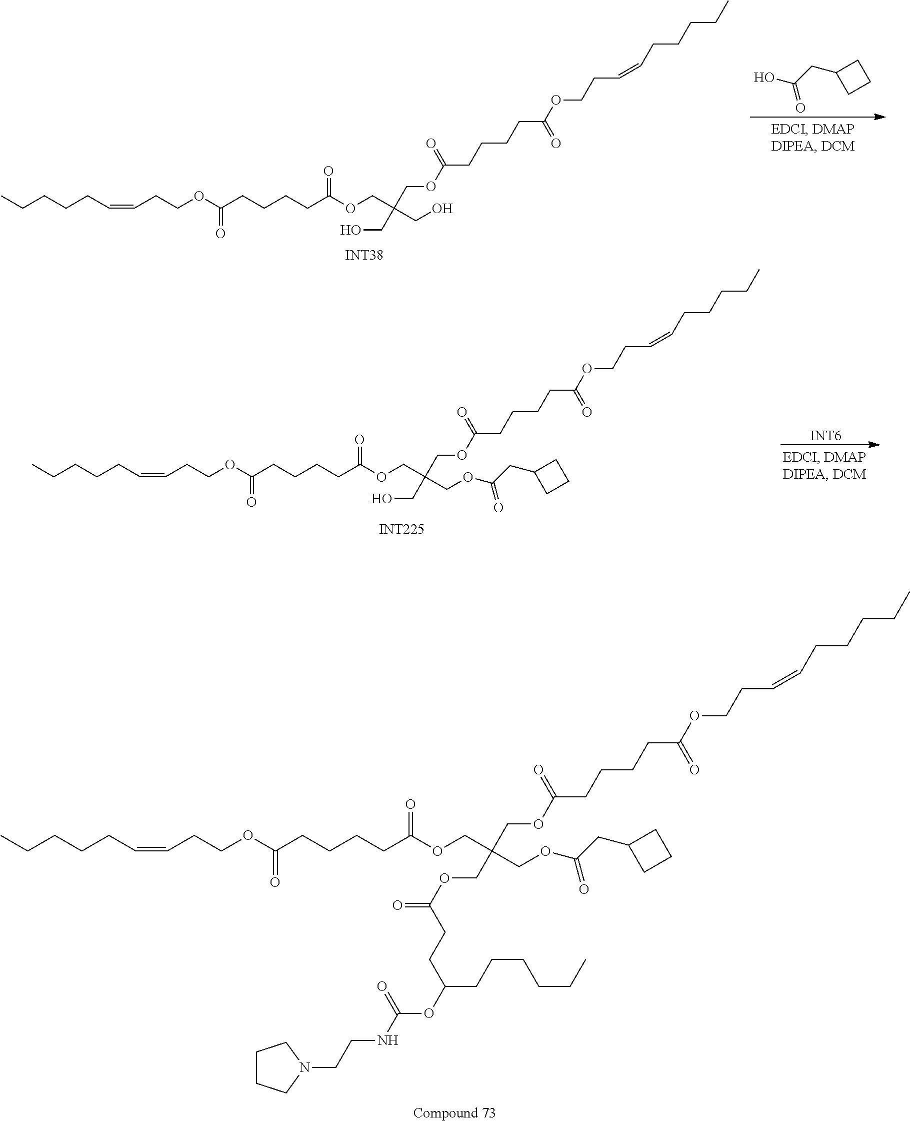

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (23 mL) was added EDCI (717.93 mg, 3.75 mmol, 1.2 eq), DMAP (38.13 mg, 312.09 mol, 0.1 eq), 2-cyclobutylacetic acid (356.22 mg, 3.12 mmol, 1 eq) and DIPEA (1.01 g, 7.80 mmol, 1.36 mL, 2.5 eq) under N₂ atmosphere. The reaction mixture was stirred at 25° C. for 10 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O6-[2-[(2-cyclobutylacetyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1.1 g, 1.49 mmol, 47.83% yield) was obtained as a colorless oil. ¹H NMR (400 MHz, CDCl₃) δ ppm 5.55-5.30 (m, 4H), 4.12-4.05 (m, 10H), 3.50 (s, 2H), 2.71-2.61 (m, 1H), 2.44 (d, J=7.6 Hz, 2H), 2.40-2.31 (m, 12H), 2.17-2.09 (m, 2H), 2.07-2.00 (m, 4H), 1.95-1.81 (m, 2H), 1.73-1.64 (m, 10H), 1.40-1.27 (m, 12H), 0.90 (t, J=6.8 Hz, 6H).

### Step 2:

To a solution of O6-[2-[(2-cyclobutylacetyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1.1 g, 1.49 mmol, 1 eq) in DCM (16 mL) was added EDCI (343.36 mg, 1.79 mmol, 1.2 eq), DMAP (18.23 mg, 149.26 mol, 0.1 eq), 4-(2-pyrrolidin-1-ylethylcarbamoyloxy)decanoic acid (588.29 mg, 1.79 mmol, 1.2 eq) and DIPEA (482.27 mg, 3.73 mmol, 649.96 L, 2.5 eq) under N₂ atmosphere. The reaction mixture was stirred at 25° C. for 10 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(2-cyclobutylacetyl)oxymethyl]-2-[[6-[(Z)-non-3-enoxy]-6-oxo-hexanoyl]oxymethyl]-3-[4-(2-pyrrolidin-1-ylethylcarbamoyloxy) decanoyloxy]propyl]O1-[(Z)-non-3-enyl]hexanedioate (0.15 g, 143.21 mol, 9.59% yield) was obtained as a pale yellow oil. H NMR (400 MHz, CDCl₃) δ ppm 5.56-5.28 (m, 4H), 5.17 (s, 1H), 4.75 (s, 1H), 4.12-4.05 (m, 12H), 3.29 (d, J=5.4 Hz, 1H), 2.67-2.51 (m, 6H), 2.44-2.31 (m, 16H), 2.16-2.01 (m, 6H), 1.97-1.72 (m, 9H), 1.72-1.41 (m, 14H), 1.38-1.26 (m, 20H), 0.91-0.86 (m, 9H).

### Example 80—Compound 77

### O6-[2-[(2-cuban-1-ylacetyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

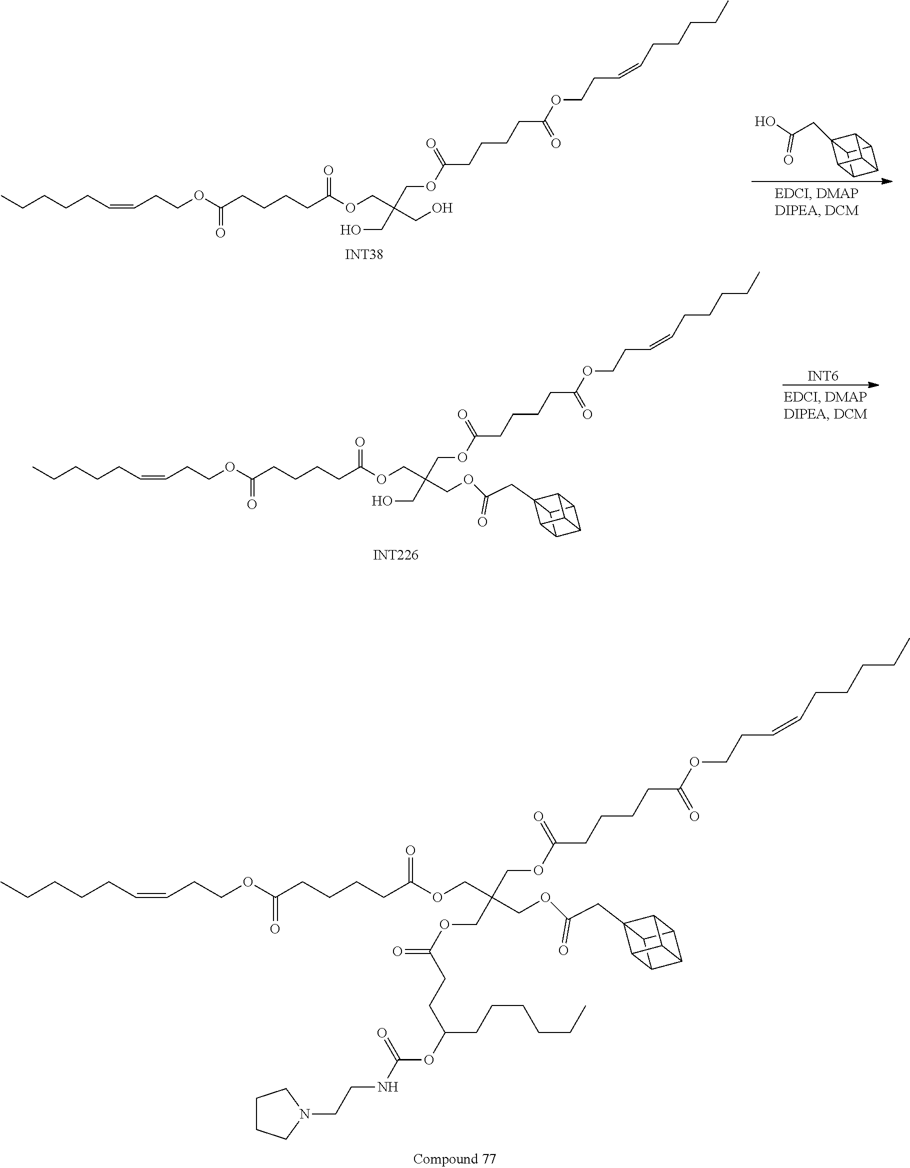

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 2-cuban-1-ylacetic acid (303.70 mg, 1.87 mmol, 0.6 eq), EDCI (717.93 mg, 3.75 mmol, 1.2 eq), DMAP (38.13 mg, 312.09 mol, 0.1 eq) and DIPEA (1.01 g, 7.80 mmol, 1.36 mL, 2.5 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(2-cuban-1-ylacetyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.08 mmol, 34.69% yield) was obtained as a colorless oil. ¹H NMR (400 MHz, CDCl₃) δ ppm 5.57-5.45 (m, 2H), 5.40-5.27 (m, 2H), 4.14-4.05 (m, 10H), 4.05-4.01 (m, 1H), 3.94-3.89 (m, 3H), 3.86-3.81 (m, 3H), 3.51 (s, 2H), 2.69 (s, 2H), 2.40-2.31 (m, 12H), 2.09-2.00 (m, 4H), 1.69-1.64 (m, 8H), 1.38-1.27 (m, 12H), 0.90 (t, J=6.8 Hz, 6H).

### Step 2:

A mixture of O6-[2-[(2-cuban-1-ylacetyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (445 mg, 566.87 mol, 1 eq), 4-(2-pyrrolidin-1-ylethylcarbamoyloxy)decanoic acid (223.42 mg, 680.24 mol, 1.2 eq), EDCI (130.40 mg, 680.24 mol, 1.2 eq), DMAP (6.93 mg, 56.69 mol, 0.1 eq) and DIPEA (183.16 mg, 1.42 mmol, 246.85 μL, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(2-cuban-1-ylacetyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 135.56 μmol, 23.91% yield, 99% purity) was obtained as a colorless oil. ¹H NMR (400 MHz, CDCl₃) δ ppm 5.55-5.48 (m, 2H), 5.38-5.31 (m, 2H), 5.26-4.93 (m, 1H), 4.87-4.68 (m, 1H), 4.14-4.01 (m, 14H), 3.92 (d, J=4.6 Hz, 2H), 3.84-3.82 (m, 2H), 3.31 (d, J=1.8 Hz, 2H), 2.68 (s, 2H), 2.65-2.43 (m, 6H), 2.38 (d, J=7.2 Hz, 6H), 2.34 (d, J=7.2 Hz, 8H), 2.08-2.02 (m, 4H), 1.98-1.84 (m, 2H), 1.80 (d, J=3.0 Hz, 4H), 1.67-1.63 (m, 8H), 1.60-1.46 (m, 4H), 1.36-1.26 (m, 22H), 0.93-0.85 (m, 12H).

### Example 81—Compound 80

### O6-[2-[2-(1-bicyclo[1.1.1]pentanyl) propanoyloxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

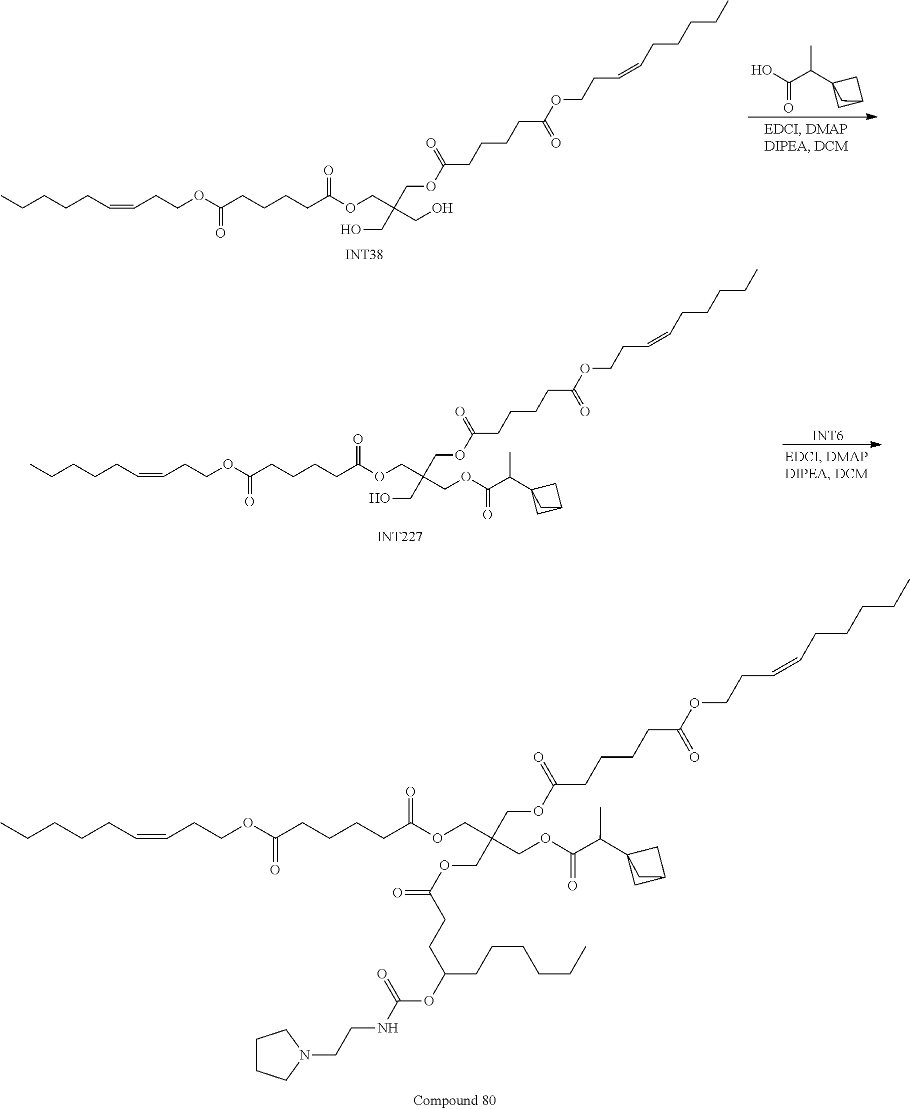

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (18 mL) was added EDCI (538.45 mg, 2.81 mmol, 1.2 eq), DMAP (28.60 mg, 234.07 mol, 0.1 eq), 2-(1-bicyclo[1.1.1]pentanyl)propanoic acid (328.11 mg, 2.34 mmol, 1 eq) and DIPEA (756.27 mg, 5.85 mmol, 1.02 mL, 2.5 eq) under N₂ atmosphere. The reaction mixture was stirred at 25° C. for 10 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[2-(1-bicyclo[1.1.1]pentanyl) propanoyloxymethyl]-2-(hydroxymethyl)-3-[6-[(Z)-non-3-enoxy]-6-oxohexanoy-1]oxy-propyl]O1-[(Z)-non-3-enyl]hexanedioate (0.8 g, 1.05 mmol, 44.79% yield) was obtained as a colorless oil. ¹H NMR (400 MHz, CDCl₃) δ ppm 5.56-5.28 (m, 4H), 4.14-4.05 (m, 10H), 3.52 (s, 2H), 2.62-2.55 (m, 1H), 2.50 (s, 1H), 2.41-2.31 (m, 12H), 2.08-2.01 (m, 4H), 1.70 (s, 6H), 1.68-1.64 (m, 8H), 1.58 (s, 1H), 1.41-1.27 (m, 12H), 1.09 (d, J=7.0 Hz, 3H), 0.90 (t, J=6.8 Hz, 6H).

### Step 2:

To a solution of O6-[2-[2-(1-bicyclo[1.1.1]pentanyl)propanoyloxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 1.05 mmol, 1 eq) in DCM (12 mL) was added EDCI (241.19 mg, 1.26 mmol, 1.2 eq), DMAP (12.81 mg, 104.85 mol, 0.1 eq), 4-(2-pyrrolidin-1-ylethylcarbamoyloxy)decanoic acid (413.24 mg, 1.26 mmol, 1.2 eq) and DIPEA (338.77 mg, 2.62 mmol, 456.57 L, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H2O (0.04% HCl)-ACN:THF=1:1]; gradient: 35%-75% B over 12.0 min) to get compound O6-[2-[2-(1-bicyclo[1.1.1]pentanyl) propanoyloxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.16 g, 279.48 mol, 26.66% yield, HCl salt) as a colorless oil. ¹H NMR (400 MHz, CD₃OD) δ ppm 5.59- 5.28 (m, 4H), 4.77 (d, J=2.8 Hz, 1H), 4.24-4.05 (m, 12H), 3.73 (s, 2H), 3.60 (d, J=5.0 Hz, 1H), 3.54-3.42 (m, 2H), 3.36-3.32 (m, 2H), 3.18-3.10 (m, 2H), 2.62-2.47 (m, 2H), 2.43-2.32 (m, 14H), 2.21-2.14 (m, 2H), 2.10-2.01 (m, 6H), 1.99-1.92 (m, 1H), 1.84-1.77 (m, 1H), 1.71 (s, 6H), 1.66-1.54 (m, 10H), 1.42-1.28 (m, 20H), 1.07 (d, J=6.8 Hz, 3H), 0.97-0.85 (m, 9H).

### Example 82—Compound 81

### O6-[2-[(6,6-difluorobicyclo[3.1.0]hexane-3-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

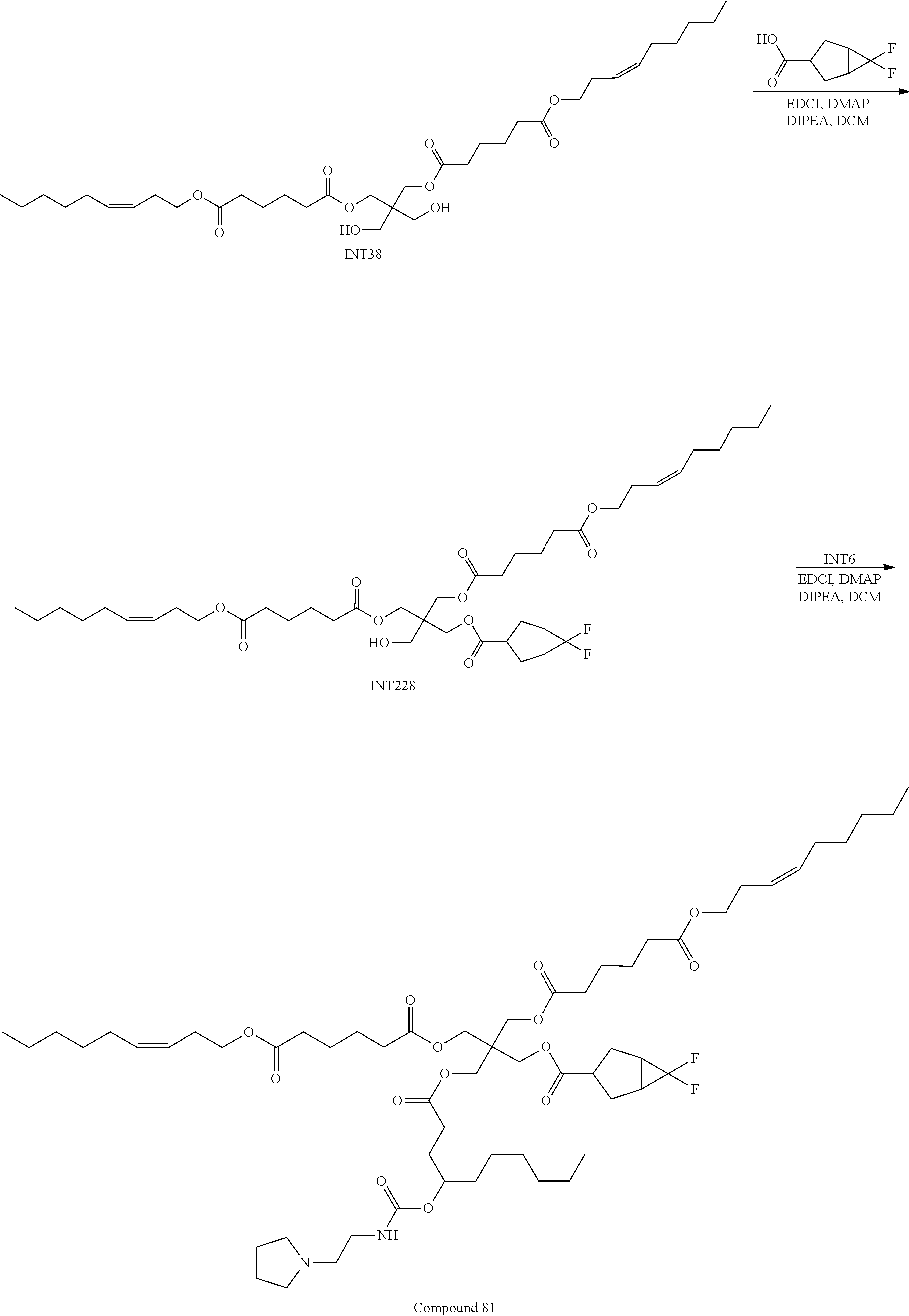

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), 6,6-difluorobicyclo[3.1.0]hexane-3-carboxylic acid (379.50 mg, 2.34 mmol, 1 eq) and DMAP (28.60 mg, 234.07 mol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(6,6-difluorobicyclo[3.1.0]hexane-3-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1.2 g, 1.53 mmol, 65.31% yield) was obtained as a colorless oil. ¹H NMR (400 MHz, CDCl₃) δ ppm 5.58-5.45 (m, 2H), 5.40-5.26 (m, 2H), 4.17-4.01 (m, 10H), 3.51 (s, 2H), 2.96-2.80 (m, 1H), 2.59 (s, 1H), 2.43-2.27 (m, 14H), 2.27-2.18 (m, 2H), 2.08-1.97 (m, 6H), 1.69-1.62 (m, 8H), 1.41-1.26 (m, 12H), 0.89 (t, J=6.8 Hz, 6H).

### Step 2:

To a solution of O6-[2-[(6,6-difluorobicyclo[3.1.0]hexane-3-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1.2 g, 1.53 mmol, 1 eq) in DCM (12 mL) was added EDCI (351.67 mg, 1.83 mmol, 1.2 eq), DIPEA (493.94 mg, 3.82 mmol, 665.69 L, 2.5 eq), 4-(2-pyrrolidin-1-ylethylcarbamoyloxy)decanoic acid (602.53 mg, 1.83 mmol, 1.2 eq) and DMAP (18.68 mg, 152.87 mol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: Phenomenex Luna C18 100*30 mm*3 um; mobile phase: [H₂O (0.04% HCl)-ACN:THF=1:1]; gradient: 40%-80% B over 12.0 min) to get compound O6-[2-[(6,6-difluorobicyclo[3.1.0]hexane-3-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 136.94 mol, 8.96% yield, HCl) as a colorless oil. ¹H NMR (400 MHz, CDCl₃) δ ppm 5.58-5.45 (m, 2H), 5.40-5.29 (m, 2H), 5.24-5.13 (m, 1H), 4.75 (d, J=2.8 Hz, 1H), 4.14-4.05 (m, 12H), 3.29 (s, 2H), 2.90-2.77 (m, 1H), 2.69-2.46 (m, 6H), 2.41-2.27 (m, 16H), 2.26-2.18 (m, 2H), 2.09-1.99 (m, 6H), 1.93-1.76 (m, 6H), 1.69-1.56 (m, 10H), 1.40-1.25 (m, 22H), 0.91-0.86 (m, 9H).

### Example 83—Compound 82

### O6-[2-[[2-(3-fluoro-1-bicyclo[1.1.1]pentanyl)acetyl]oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate

### Synthetic Scheme:

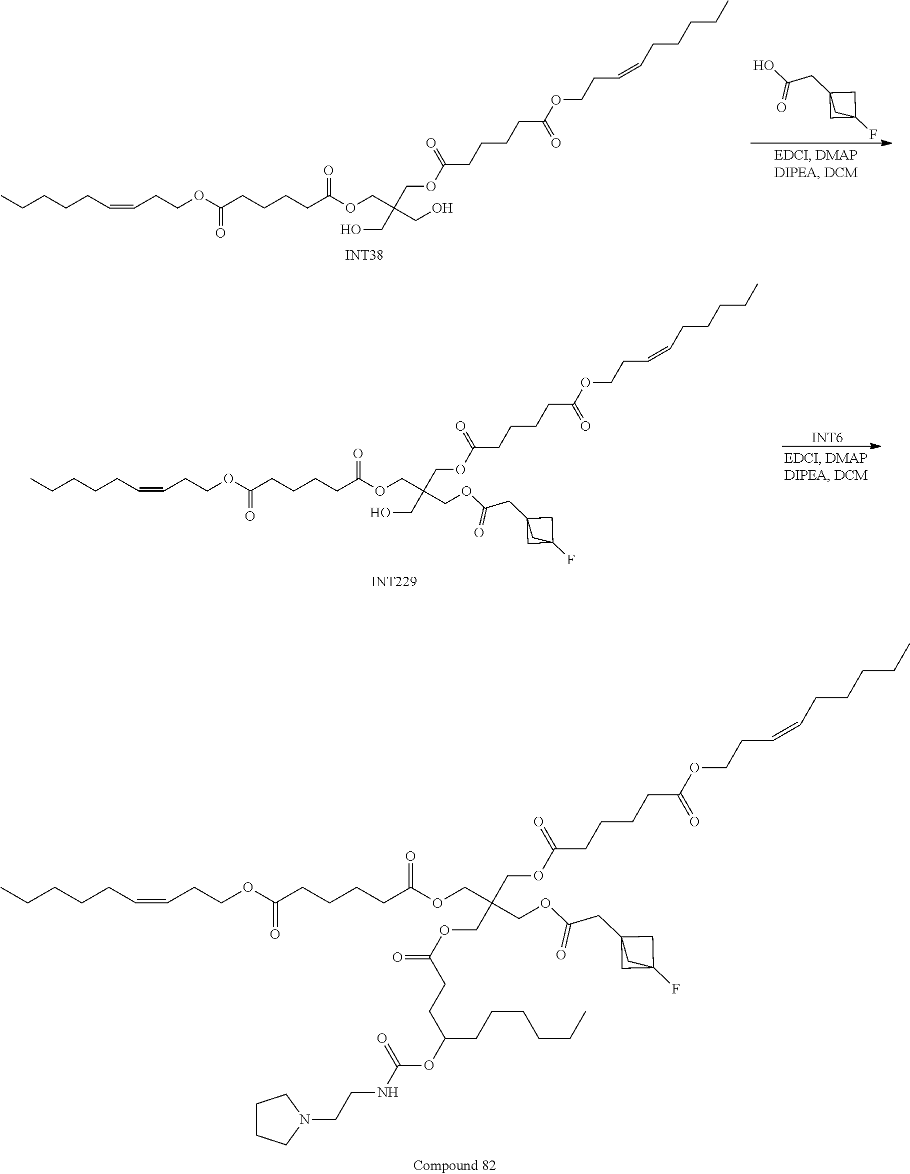

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 2-(3-fluoro-1-bicyclo[1.1.1]pentanyl)acetic acid (269.91 mg, 1.87 mmol, 0.6 eq), DMAP (38.13 mg, 312.09 mol, 0.1 eq), DIPEA (1.01 g, 7.80 mmol, 1.36 mL, 2.5 eq) and EDCI (717.93 mg, 3.75 mmol, 1.2 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[2-(3-fluoro-1-bicyclo[1.1.1]pentanyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.11 mmol, 35.51% yield) was obtained as a colorless oil. ¹H NMR (400 MHz, CDCl₃) δ ppm 5.56-5.47 (m, 2H), 5.38-5.31 (m, 2H), 4.12 (s, 6H), 4.07 (t, J=6.8 Hz, 4H), 3.52 (s, 2H), 2.71 (s, 2H), 2.40-2.31 (m, 12H), 2.03 (s, 10H), 1.69-1.64 (m, 8H), 1.38-1.26 (m, 12H), 0.90 (t, J=6.8 Hz, 6H).

### Step 2:

A mixture of O6-[2-[[2-(3-fluoro-1-bicyclo[1.1.1]pentanyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.11 mmol, 1 eq), 4-(2-pyrrolidin-1-ylethylcarbamoyloxy)decanoic acid (436.80 mg, 1.33 mmol, 1.2 eq), EDCI (254.94 mg, 1.33 mmol, 1.2 eq), DMAP (13.54 mg, 110.83 mol, 0.1 eq) and DIPEA (358.08 mg, 2.77 mmol, 482.59 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 45%-75% B over 12.0 min) to get compound O6-[2-[[2-(3-fluoro-1-bicyclo[1.1.1]pentanyl)acetyl]oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate (0.15 g, 139.22 mol, 12.56% yield, 100% purity, HCl salt) was obtained as a colorless oil. ¹H NMR (400 MHz, CDCl₃) δ ppm 12.60-11.90 (m, 1H), 6.38 (s, 1H), 5.56-5.47 (m, 2H), 5.39-5.30 (m, 2H), 4.79-4.68 (m, 1H), 4.19-4.05 (m, 12H), 3.90-3.83 (m, 2H), 3.66 (d, J=6.4 Hz, 2H), 3.31-3.20 (m, 2H), 2.90-2.80 (m, 2H), 2.71 (s, 2H), 2.41-2.32 (m, 14H), 2.28-2.22 (m, 2H), 2.11-2.02 (m, 12H), 1.93-1.88 (m, 1H), 1.82-1.77 (m, 1H), 1.65 (d, J=2.8 Hz, 8H), 1.49 (d, J=6.8 Hz, 1H), 1.39-1.26 (m, 20H), 0.92-0.86 (m, 9H).

### Example 84—Compound 86

### O6-[2-[[6-[(Z)-non-3-enoxy]-6-oxo-hexanoyl]oxymethyl]-2-[[2-(8-oxabicyclo[3.2.1]octan-3-yl)acetyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

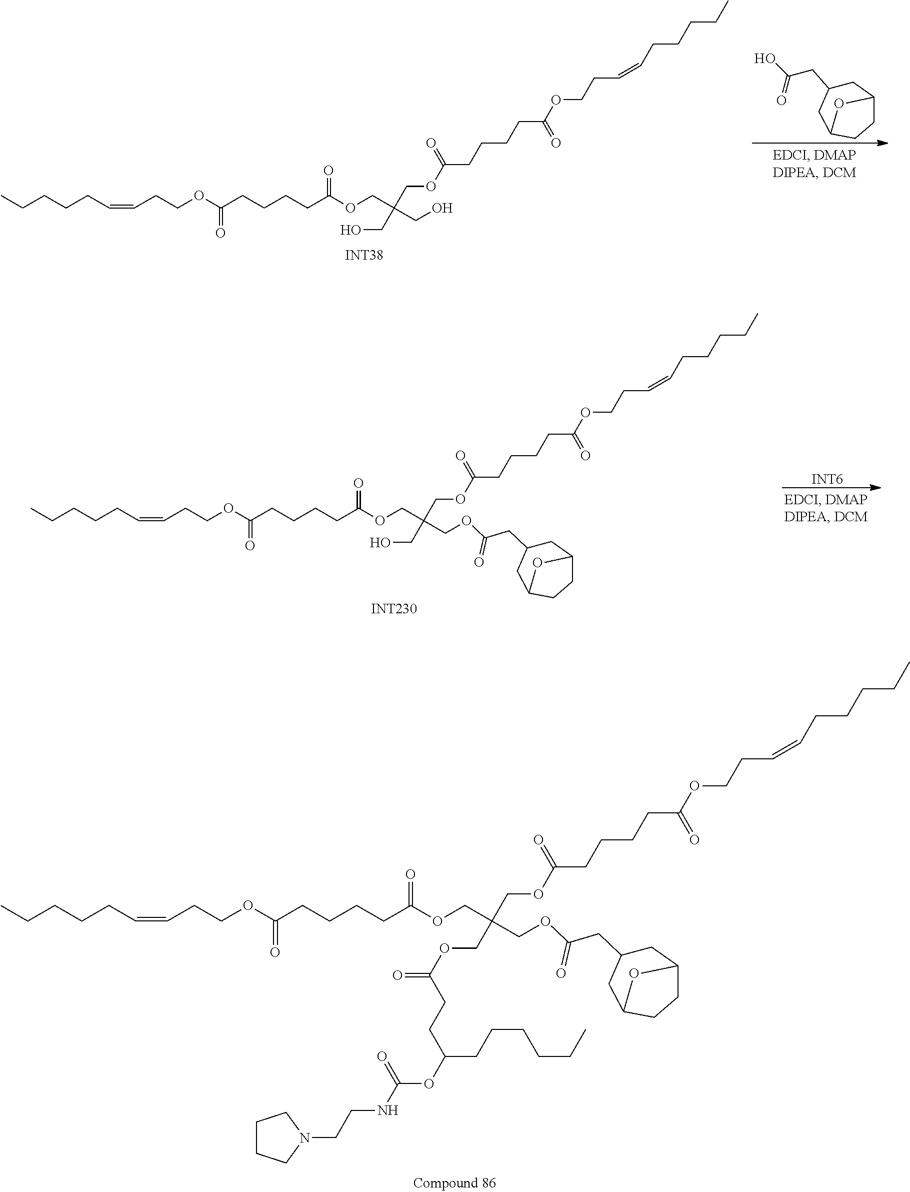

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), 4-pyrrolidin-1-ylpyridine (34.69 mg, 234.07 mol, 0.1 eq) and 2-(8-oxabicyclo[3.2.1]octan-3-yl)acetic acid (398.39 mg, 2.34 mmol, 1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[[6-[(Z)-non-3-enoxy]-6-oxo-hexanoyl]oxymethyl]-3-[2-(8-oxabicyclo[3.2.1]octan-3-yl)acetyl]oxy-propyl]O1-[(Z)-non-3-enyl]hexanedioate (0.8 g, 1.01 mmol, 43.10% yield) was obtained as a colorless oil. ¹H NMR (400 MHz, CDCl₃) δ ppm 5.59-5.44 (m, 2H), 5.41-5.27 (m, 2H), 4.36 (br s, 2H), 4.14-4.05 (m, 10H), 3.61-3.44 (m, 2H), 2.60 (t, J=6.0 Hz, 1H), 2.54-2.46 (m, 2H), 2.42-2.31 (m, 12H), 2.23-2.16 (m, 2H), 2.10-1.97 (m, 6H), 1.78-1.73 (m, 2H), 1.70-1.61 (m, 8H), 1.58 (s, 1H), 1.40-1.20 (m, 14H), 0.90 (t, J=6.8 Hz, 6H).

### Step 2:

To a solution of O6-[2-(hydroxymethyl)-2-[[6-[(Z)-non-3-enoxy]-6-oxo-hexanoyl]oxymethyl]-3-[2-(8-oxabicyclo[3.2.1]octan-3-yl)acetyl]oxy-propyl]O1-[(Z)-non-3-enyl]hexanedioate (0.8 g, 252.20 mol, 1 eq) in DCM (8 mL) was added EDCI (232.08 mg, 1.21 mmol, 1.2 eq), DIPEA (325.96 mg, 2.52 mmol, 439.28 μL, 2.5 eq), DMAP (12.32 mg, 100.88 mol, 0.1 eq) and 4-(2-pyrrolidin-1-ylethylcarbamoyloxy)decanoic acid (331.32 mg, 1.01 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[6-[(Z)-non-3-enoxy]-6-oxo-hexanoyl]oxymethyl]-2-[[2-(8-oxabicyclo[3.2.1]octan-3-yl)acetyl]oxymethyl]-3-[4-(2-pyrrolidin-1-ylethylcarbamoyloxy)decanoyloxy]propyl]O1-[(Z)-non-3-enyl]hexanedioate (0.15 g, 136.11 mol, 13.47% yield) was obtained as a colorless oil. ¹H NMR (400 MHz, CDCl₃) δ ppm 5.58-5.46 (m, 2H), 5.40-5.30 (m, 2H), 5.29-5.03 (m, 1H), 4.80-4.67 (m, 1H), 4.35 (s, 2H), 4.15-4.03 (m, 12H), 3.30 (s, 2H), 2.72-2.45 (m, 8H), 2.42-2.30 (m, 14H), 2.22-2.15 (m, 2H), 2.08-1.95 (m, 6H), 1.88-1.72 (m, 8H), 1.68-1.63 (m, 8H), 1.55-1.46 (m, 2H), 1.39-1.19 (m, 22H), 0.96-0.81 (m, 9H).

### Example 85—Compound 87

### O6-[2-(bicyclo[3.1.0]hexane-3-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

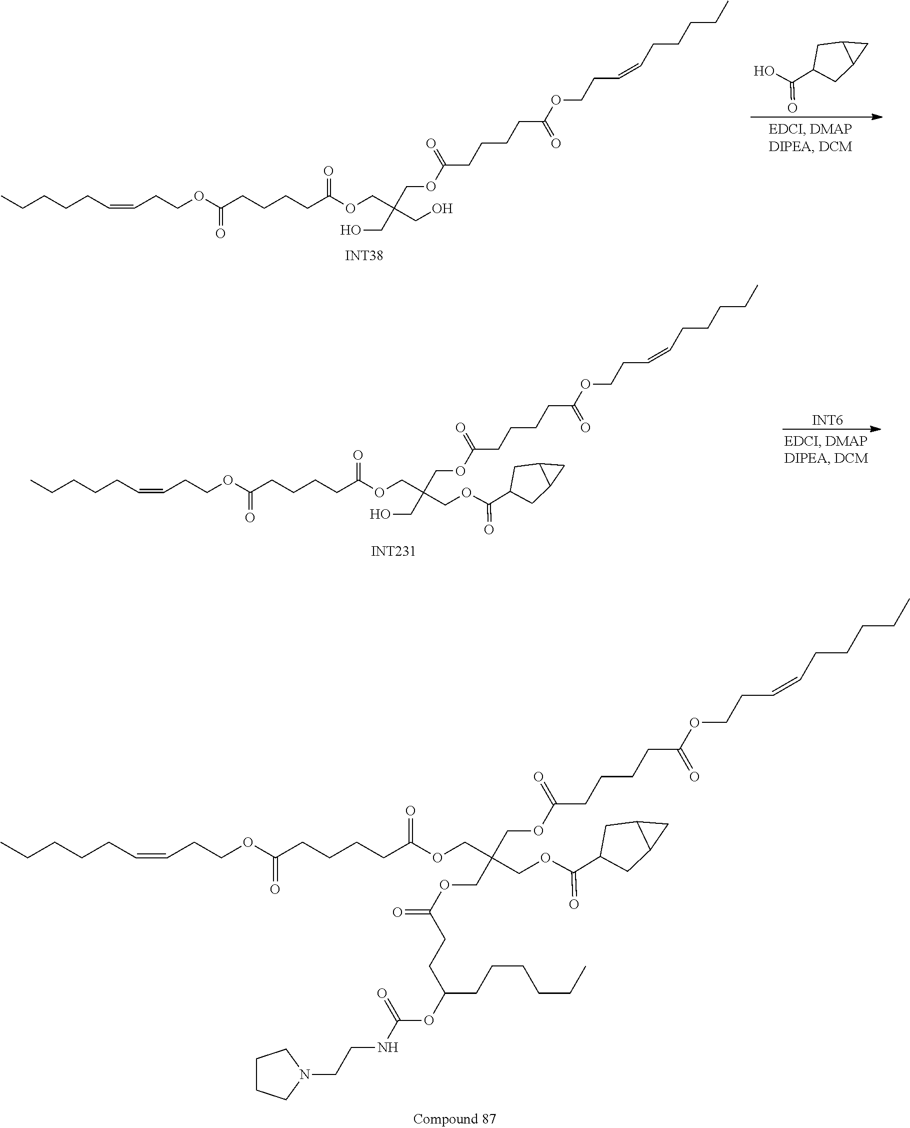

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), DMAP (28.60 mg, 234.07 mol, 0.1 eq) and bicyclo[3.1.0]hexane-3-carboxylic acid (295.28 mg, 2.34 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[3.1.0]hexane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.34 mmol, 57.04% yield) was obtained as a colorless oil. ¹H NMR (400 MHz, CDCl₃) δ ppm 5.61-5.45 (m, 2H), 5.41-5.26 (m, 2H), 4.17-4.01 (m, 10H), 3.51 (s, 2H), 2.97-2.84 (m, 1H), 2.66-2.54 (m, 1H), 2.43-2.29 (m, 12H), 2.22-1.95 (m, 8H), 1.71-1.62 (m, 8H), 1.56 (s, 2H), 1.42-1.21 (m, 14H), 0.90 (t, J=6.8 Hz, 6H).

### Step 2:

To a solution of O6-[2-(bicyclo[3.1.0]hexane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.34 mmol, 1 eq) in DCM (10 mL) was added EDCI (307.14 mg, 1.60 mmol, 1.2 eq), DIPEA (431.39 mg, 3.34 mmol, 581.39 L, 2.5 eq), 4-(2-pyrrolidin-1-ylethylcarbamoyloxy)decanoic acid (526.23 mg, 1.60 mmol, 1.2 eq) and DMAP (16.31 mg, 133.51 mol, 0.1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 45%-75% B over 12.0 min) to get compound O6-[2-(bicyclo[3.1.0]hexane-3-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 141.59 mol, 10.60% yield, HCl salt) as a colorless oil. ¹H NMR (400 MHz, CD₃OD) δ ppm 5.57-5.46 (m, 2H), 5.43-5.31 (m, 2H), 4.83-4.70 (m, 1H), 4.21-4.03 (m, 12H), 3.74 (s, 2H), 3.55-3.45 (m, 2H), 3.36-3.32 (m, 2H), 3.19-3.10 (m, 2H), 3.00-2.87 (m, 1H), 2.43-2.31 (m, 14H), 2.23-1.90 (m, 14H), 1.85-1.76 (m, 1H), 1.69-1.54 (m, 10H), 1.42-1.27 (m, 22H), 0.98-0.85 (m, 9H), 0.44-0.31 (m, 1H), −0.09-−0.20 (m, 1H).

### Example 86—Compound 88

### O6-[2-[[6-[(Z)-non-3-enoxy]-6-oxo-hexanoyl]oxymethyl]-2-(8-oxabicyclo[3.2.1]octane-3-carbonyloxymethyl)-3-[4-(2-pyrrolidin-1-ylethylcarbamoyloxy)decanoyloxy]propyl]O1-[(Z)-non-3-enyl]hexanedioate

### Synthetic Scheme:

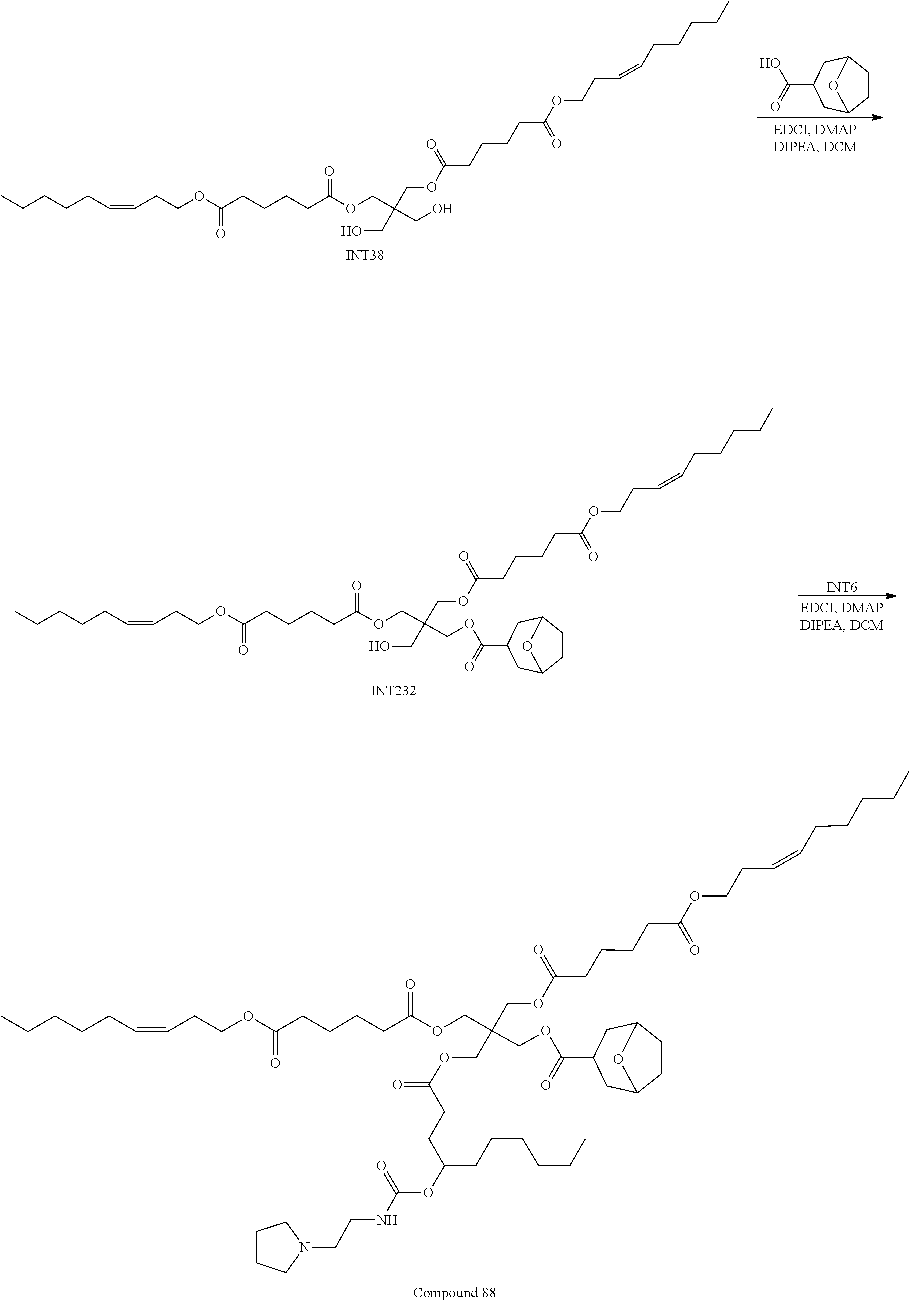

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), 8-oxabicyclo[3.2.1]octane-3-carboxylic acid (365.56 mg, 2.34 mmol, 1 eq), DIPEA (756.28 mg, 5.85 mmol, 1.02 mL, 2.5 eq) and DMAP (28.60 mg, 234.07 mol, 0.1 eq) under N₂ atmosphere. The reaction mixture was stirred at 25° C. for 10 hr under N₂ atmosphere. The reaction mixture was diluted with H₂O (50 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[[6-[(Z)-non-3-enoxy]-6-oxo-hexanoyl]oxymethyl]-3-(8-oxabicyclo[3.2.1]octane-3-carbonyloxy)propyl]O1-[(Z)-non-3-enyl]hexanedioate (1 g, 1.28 mmol, 54.84% yield) was obtained as a colorless oil.

### Step 2:

To a solution of O6-[2-(hydroxymethyl)-2-[[6-[(Z)-non-3-enoxy]-6-oxo-hexanoyl]oxymethyl]-3-(8-oxabicyclo[3.2.1]octane-3-carbonyloxy)propyl]O1-[(Z)-non-3-enyl]hexanedioate (1.00 g, 1.28 mmol, 1 eq) in DCM (10 mL) was added EDCI (295.30 mg, 1.54 mmol, 1.2 eq), DIPEA (414.77 mg, 3.21 mmol, 558.99 μL, 2.5 eq), DMAP (15.68 mg, 128.37 mol, 0.1 eq) and 4-(2-pyrrolidin-1-ylethylcarbamoyloxy)decanoic acid (505.95 mg, 1.54 mmol, 1.2 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[6-[(Z)-non-3-enoxy]-6-oxo-hexanoyl]oxymethyl]-2-(8-oxabicyclo[3.2.1]octane-3-carbonyloxymethyl)-3-[4-(2-pyrrolidin-1-ylethylcarbamoyloxy)decanoyloxy]propyl]O1-[(Z)-non-3-enyl]hexanedioate (0.15 g, 137.69 mol, 10.73% yield) was obtained as a colorless oil. ¹H NMR (400 MHz, CDCl₃) δ ppm 5.59-5.46 (m, 2H), 5.41-5.29 (m, 2H), 4.75 (d, J=4.2 Hz, 1H), 4.43 (s, 1H), 4.37-4.29 (m, 1H), 4.16-4.04 (m, 12H), 3.43 (d, J=6.8 Hz, 2H), 2.86-2.60 (m, 4H), 2.43-2.30 (m, 14H), 2.14-1.84 (m, 14H), 1.82-1.42 (m, 18H), 1.41-1.25 (m, 20H), 0.96-0.81 (m, 9H).

### Example 87—Compound 89

### O6-[2-[[3-(difluoromethyl)bicyclo[1.1.1]pentane-1-carbonyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyl-oxy]propyl]O1-[(Z)-non-3-enyl]hexanedioate

### Synthetic Scheme:

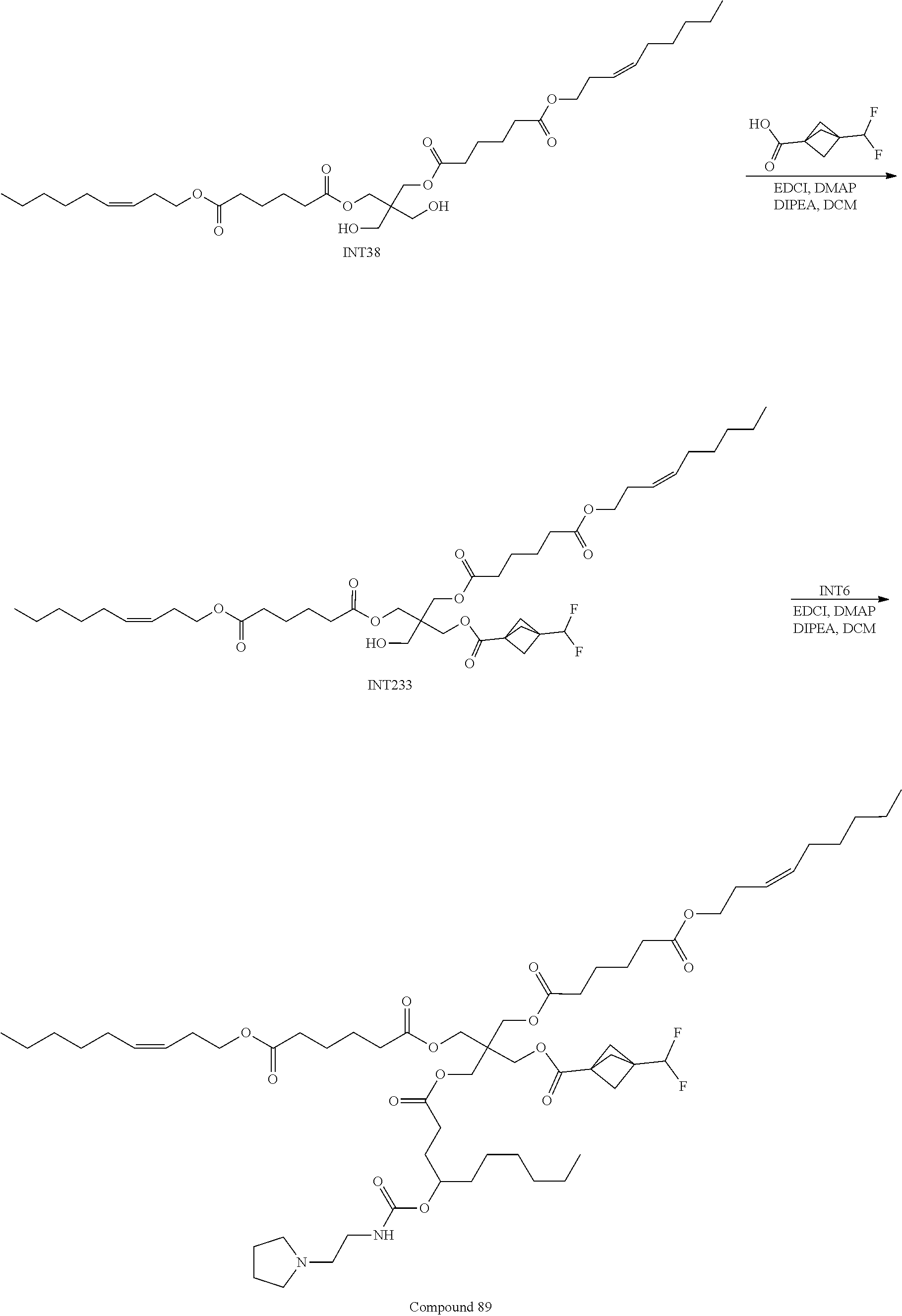

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (18 mL) was added EDCI (538.45 mg, 2.81 mmol, 1.2 eq), DMAP (28.60 mg, 234.07 mol, 0.1 eq), 3-(difluoromethyl)bicyclo[1.1.1]pentane-1-carboxylic acid (379.50 mg, 2.34 mmol, 1 eq) and DIPEA (756.28 mg, 5.85 mmol, 1.02 mL, 2.5 eq) under N₂ atmosphere. The reaction mixture was stirred at 25° C. for 10 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[3-(difluoromethyl)bicyclo[1.1.1]pentane-1-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.9 g, 1.15 mmol, 48.98% yield) was obtained as a colorless oil. ¹H NMR (400 MHz, CDCl₃) δ ppm 5.72 (t, J=56.2 Hz, 1H), 5.55-5.31 (m, 4H), 4.13-4.05 (m, 10H), 3.52 (s, 2H), 2.58 (s, 1H), 2.41-2.32 (m, 12H), 2.16 (s, 6H), 2.08-2.01 (m, 4H), 1.66 (s, 8H), 1.40-1.27 (m, 12H), 0.90 (t, J=6.8 Hz, 6H).

### Step 2:

To a solution of O6-[2-[[3-(difluoromethyl)bicyclo[1.1.1]pentane-1-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.9 g, 1.15 mmol, 1 eq) in DCM (13 mL) was added EDCI (263.75 mg, 1.38 mmol, 1.2 eq), DMAP (14.01 mg, 114.66 mol, 0.1 eq), 4-(2-pyrrolidin-1-ylethylcarbamoyloxy)decanoic acid (451.90 mg, 1.38 mmol, 1.2 eq) and DIPEA (370.46 mg, 2.87 mmol, 499.27 μL, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[3-(difluoromethyl)bicyclo[1.1.1]pentane-1-carbonyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyl-oxy]propyl]O1-[(Z)-non-3-enyl]hexanedioate (0.16 g, 146.07 mol, 12.74% yield) was obtained as a colorless oil. ¹H NMR (400 MHz, CDCl₃) δ ppm 5.71 (t, J=56.2 Hz, 1H), 5.55-5.46 (m, 2H), 5.38-5.29 (m, 2H), 5.24-5.10 (m, 1H), 4.75 (s, 1H), 4.13-4.05 (m, 12H), 3.30 (s, 2H), 2.69-2.44 (m, 6H), 2.41-2.31 (m, 14H), 2.16 (s, 6H), 2.07-2.01 (m, 4H), 1.93-1.74 (m, 6H), 1.67-1.49 (m, 12H), 1.40-1.26 (m, 20H), 0.93-0.85 (m, 9H).

### Example 88—Compound 90

### O6-[2-(bicyclo[2.1.1]hexane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

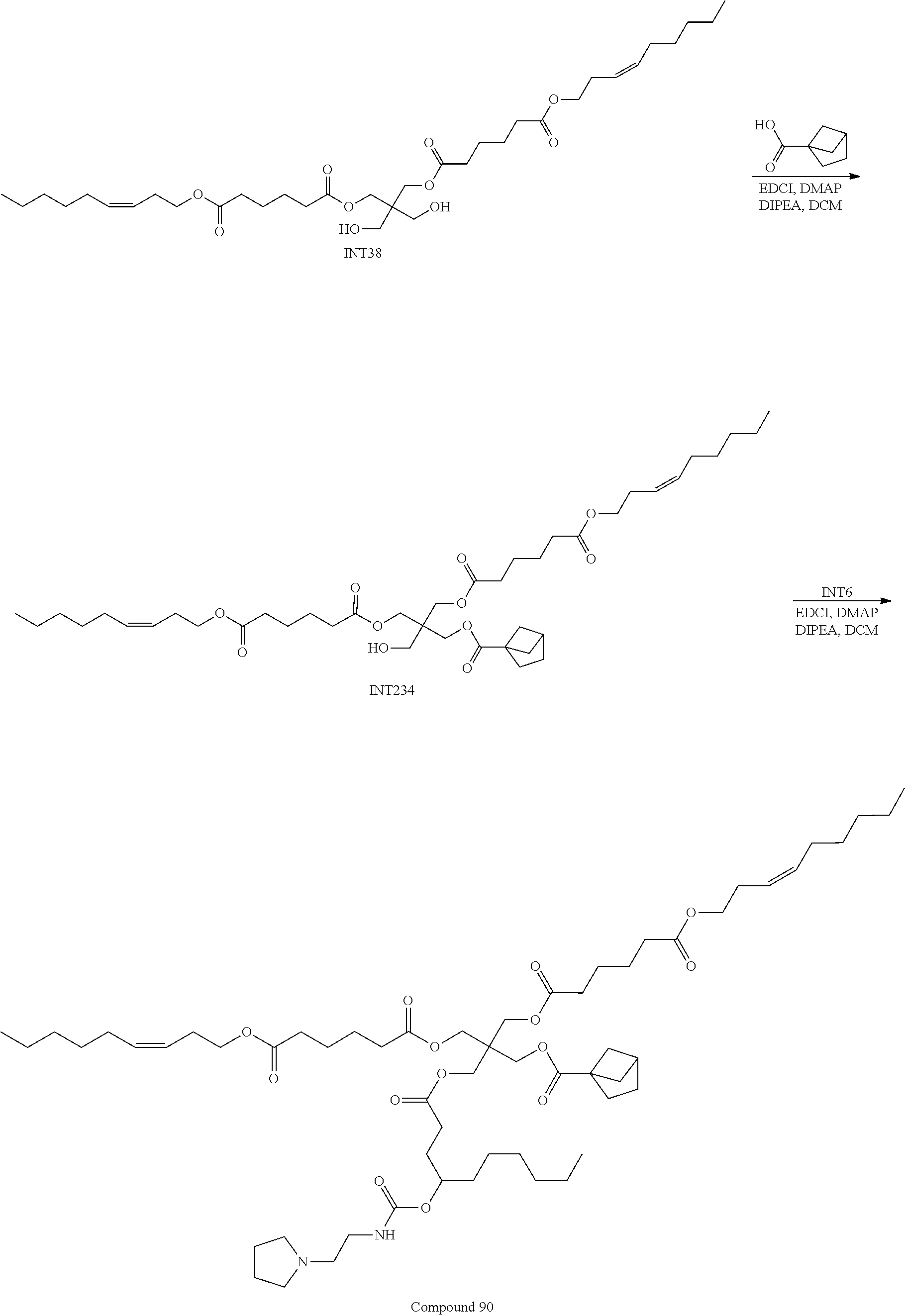

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (18 mL) was added EDCI (717.93 mg, 3.75 mmol, 1.2 eq), DMAP (38.13 mg, 312.09 mol, 0.1 eq), bicyclo[2.1.1]hexane-1-carboxylic acid (393.71 mg, 3.12 mmol, 1 eq) and DIPEA (1.01 g, 7.80 mmol, 1.36 mL, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[2.1.1]hexane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.13 mmol, 36.36% yield) was obtained as a colorless oil. ¹H NMR (400 MHz, CDCl₃) δ ppm 5.62-5.20 (m, 4H), 4.15-4.05 (m, 10H), 3.51 (s, 2H), 2.46 (s, 1H), 2.42-2.30 (m, 12H), 2.09-1.97 (m, 4H), 1.90-1.82 (m, 4H), 1.78-1.72 (m, 2H), 1.70-1.63 (m, 8H), 1.40-1.25 (m, 14H), 0.90 (t, J=6.8 Hz, 6H).

### Step 2:

To a solution of O6-[2-(bicyclo[2.1.1]hexane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.13 mmol, 1 eq) in DCM (12 mL) was added EDCI (261.07 mg, 1.36 mmol, 1.2 eq), DMAP (13.86 mg, 113.49 mol, 0.1 eq), 4-(2-pyrrolidin-1-ylethylcarbamoyloxy)decanoic acid (447.29 mg, 1.36 mmol, 1.2 eq) and DIPEA (366.69 mg, 2.84 mmol, 494.19 μL, 2.5 eq) under N₂ atmosphere. The reaction mixture was stirred at 25° C. for 10 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 40%-70% B over 20.0 min) to get compound O6-[2-(bicyclo[2.1.1]hexane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.3 g, 283.18 mol, 24.95% yield, HCl salt) as a colorless oil. ¹H NMR (400 MHz, CD₃OD) δ ppm 5.56-5.31 (m, 4H), 4.77 (d, J=2.6 Hz, 1H), 4.24-4.05 (m, 12H), 3.73 (s, 2H), 3.54-3.44 (m, 2H), 3.37-3.32 (m, 2H), 3.18-3.09 (m, 2H), 2.49-2.25 (m, 16H), 2.23-2.14 (m, 2H), 2.11-2.00 (m, 6H), 1.98-1.75 (m, 8H), 1.67-1.53 (m, 10H), 1.42-1.28 (m, 22H), 0.96-0.85 (m, 9H).

### Example 89—Compound 91

### O6-[2-[[2-(1-bicyclo[1.1.1]pentanyl)acetyl]oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate

### Synthetic Scheme:

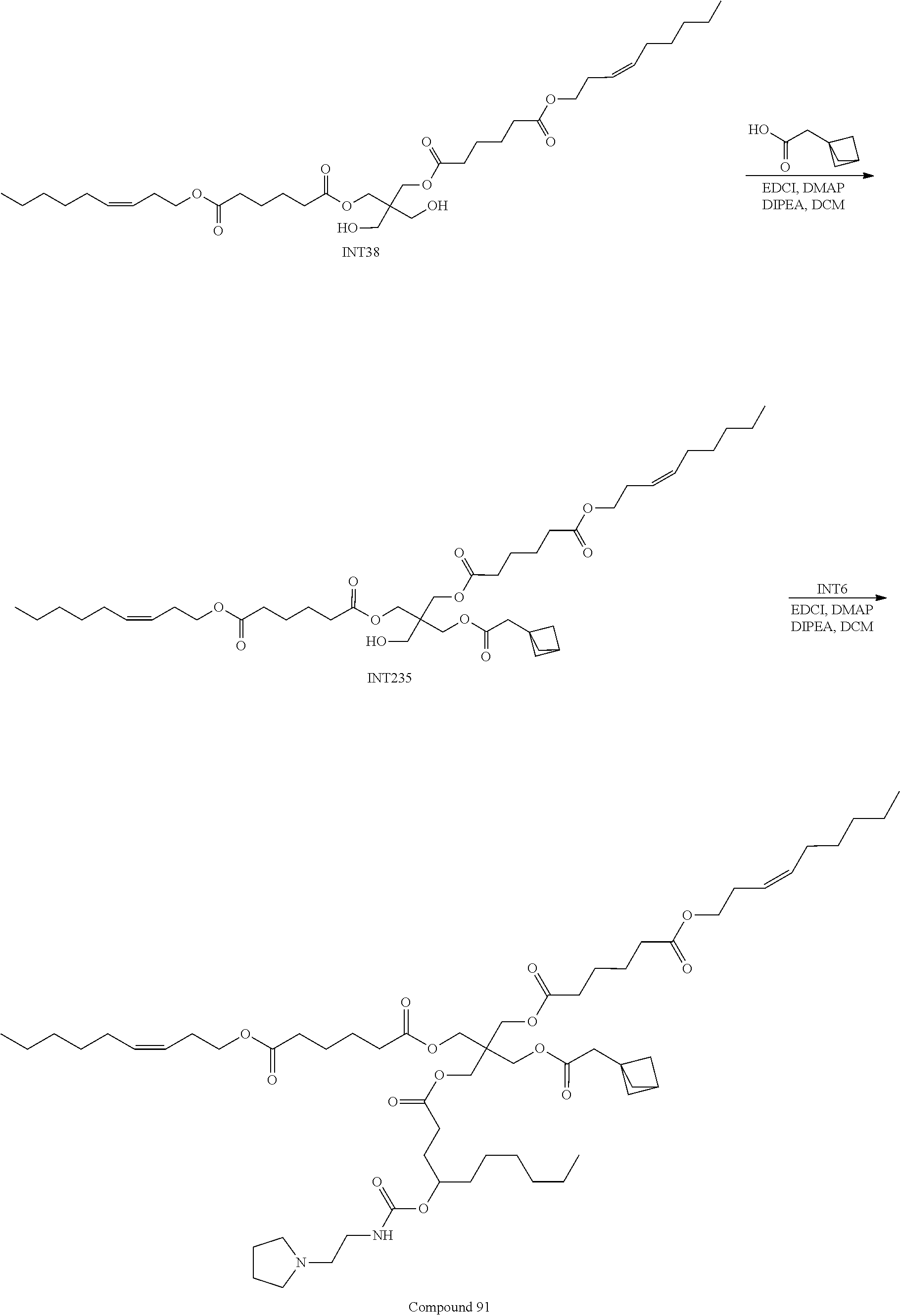

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (3 g, 4.68 mmol, 1 eq) in DCM (34 mL) was added EDCI (1.08 g, 5.62 mmol, 1.2 eq), DMAP (57.19 mg, 468.13 mol, 0.1 eq), 2-(1-bicyclo[1.1.1]pentanyl)acetic acid (472.45 mg, 3.75 mmol, 0.8 eq) and DIPEA (1.51 g, 11.70 mmol, 2.04 mL, 2.5 eq) under N₂ atmosphere. The reaction mixture was stirred at 25° C. for 10 hr under N₂ atmosphere. The reaction mixture was diluted with H₂O (50 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O6-[2-[[2-(1-bicycle[1.1.1]pentanyl) acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (1.3 g, 1.74 mmol, 37.08% yield) was obtained as a colorless oil. ¹H NMR (400 MHz, CDCl₃) δ ppm 5.55-5.30 (m, 4H), 4.14-4.10 (m, 6H), 4.07 (t, J=7.0 Hz, 4H), 3.52 (s, 2H), 2.52-2.48 (m, 3H), 2.41-2.32 (m, 12H), 2.07-2.01 (m, 4H), 1.79 (s, 6H), 1.70-1.64 (m, 8H), 1.38-1.27 (m, 12H), 0.90 (t, J=6.8 Hz, 6H).

### Step 2:

To a solution of O6-[2-[[2-(1-bicyclo[1.1.1]pentanyl)acetyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (1.3 g, 1.74 mmol, 1 eq) in DCM (16 mL) was added EDCI (399.28 mg, 2.08 mmol, 1.2 eq), DMAP (21.20 mg, 173.57 mol, 0.1 eq), 4-(2-pyrrolidin-1-ylethylcarbamoyloxy)decanoic acid (342.05 mg, 1.04 mmol, 0.6 eq) and DIPEA (560.81 mg, 4.34 mmol, 755.81 μL, 2.5 eq) under N₂ atmosphere. The reaction mixture was stirred at 25° C. for 10 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[2-(1-bicyclo[1.1.1]pentanyl)acetyl]oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate (0.15 g, 140.72 mol, 8.11% yield, 99.39% purity) was obtained as a pale yellow oil. ¹H NMR (400 MHz, CDCl₃) δ ppm 5.57-5.28 (m, 4H), 5.25-5.12 (m, 1H), 4.75 (s, 1H), 4.13-4.05 (m, 12H), 3.30 (d, J=4.0 Hz, 2H), 2.66-2.48 (m, 9H), 2.41-2.31 (m, 14H), 2.09-1.84 (m, 6H), 1.78 (s, 8H), 1.71-1.44 (m, 12H), 1.40-1.26 (m, 20H), 0.93-0.85 (m, 9H).

### Example 90—Compound 92

### O6-[2-[(3-methylbicyclo[1.1.1]pentane-1-carbonyl)oxymethyl]-2-[[6-[(Z)-non-3-enoxy]-6-oxo-hexanoyl]oxymethyl]-3-[4-(2-pyrrolidin-1ylethyl carbamoyloxy)decanoyloxy]propyl]O1-[(Z)-non-3-enyl]hexanedioate

### Synthetic Scheme:

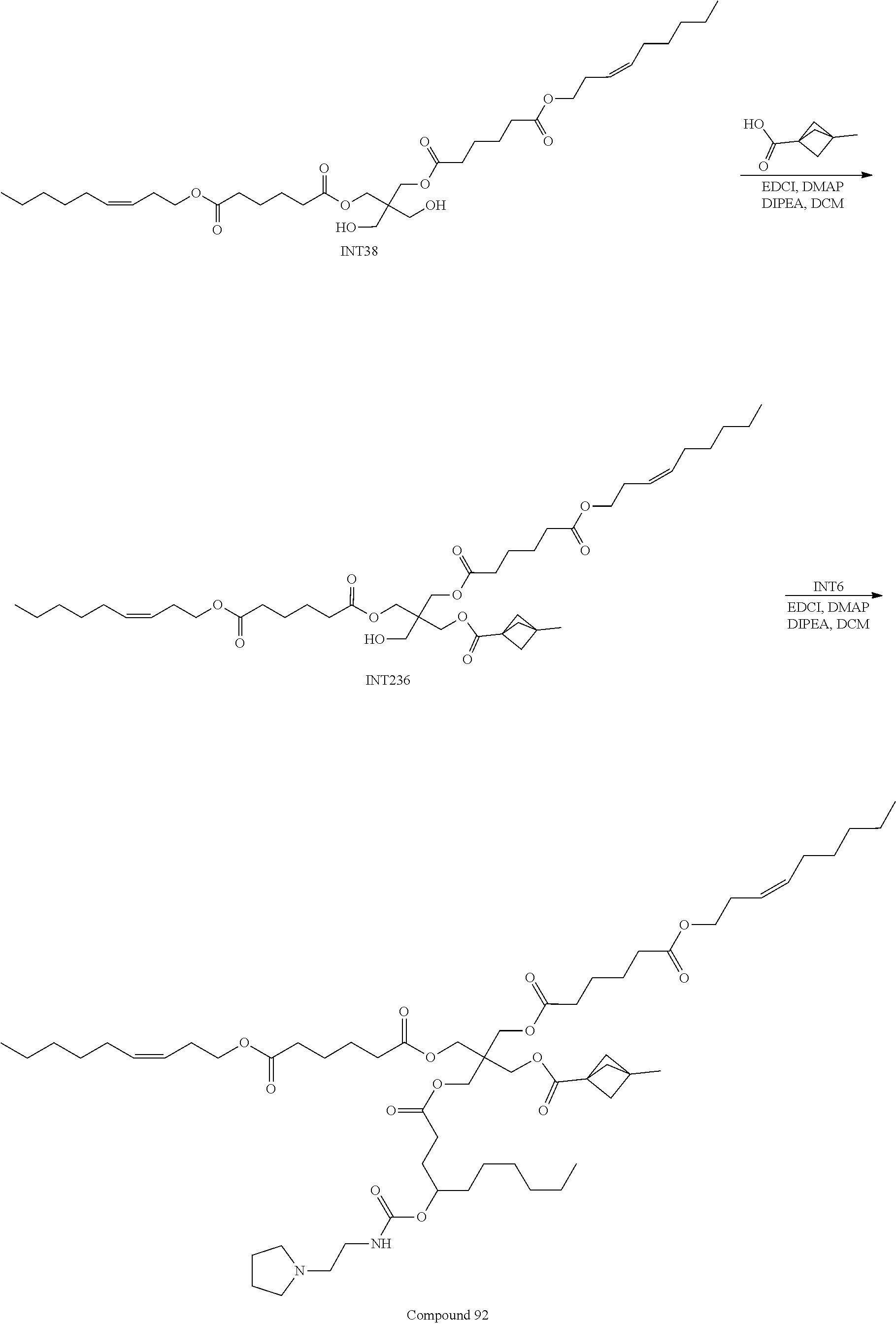

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 3-methylbicyclo[1.1.1]pentane-1-carboxylic acid (236.23 mg, 1.87 mmol, 0.6 eq), DMAP (381.27 mg, 3.12 mmol, 1 eq), EDCI (598.28 mg, 3.12 mmol, 1 eq) and DIPEA (403.35 mg, 3.12 mmol, 543.60 L, 1 eq) in DCM (22.36 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[(3-methylbicyclo[1.1.1]pentane-1-carbonyl)oxymethyl]-3-[6-[(Z)-non-3-enoxy]-6-oxo-hexanoyl]oxy-propyl]O1-[(Z)-non-3-enyl]hexanedioate (0.85 g, 1.13 mmol, 36.36% yield) was obtained as a colorless oil. ¹H NMR (400 MHz, CDCl₃) δ ppm 5.55-5.47 (m, 2H), 5.38-5.30 (m, 2H), 4.12-4.05 (m, 10H), 3.50 (s, 2H), 2.40-2.31 (m, 12H), 2.04 (d, J=7.0 Hz, 4H), 1.93 (s, 6H), 1.69-1.64 (m, 8H), 1.39-1.27 (m, 12H), 1.19 (s, 3H), 0.90 (t, J=6.8 Hz, 6H).

### Step 2:

A mixture of O6-[2-(hydroxymethyl)-2-[(3-methylbicyclo[1.1.1]pentane-1-carbonyl)oxymethyl]-3-[6-[(Z)-non-3-enoxy]-6-oxo-hexanoyl]oxy-propyl]O1-[(Z)-non-3-enyl]hexanedioate (0.85 g, 1.13 mmol, 1 eq), 4-(2-pyrrolidin-1-ylethylcarbamoyloxy)decanoic acid (223.65 mg, 680.93 mol, 0.6 eq), EDCI (261.07 mg, 1.36 mmol, 1.2 eq), DMAP (13.86 mg, 113.49 mol, 0.1 eq) and DIPEA (366.69 mg, 2.84 mmol, 494.19 L, 2.5 eq) in DCM (10.73 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 50%-80% B over 12.0 min) to get compound O6-[2-[(3-methylbicyclo[1.1.1]pentane-1-carbonyl)oxymethyl]-2-[[6-[(Z)-non-3-enoxy]-6-oxo-hexanoyl]oxymethyl]-3-[4-(2-pyrrolidin-1ylethyl carbamoyloxy)decanoyloxy]propyl]O1-[(Z)-non-3-enyl]hexanedioate (0.15 g, 141.59 mol, 12.48% yield, 100% purity, HCl salt) as a colorless oil. ¹H NMR (400 MHz, CDCl₃) δ ppm 13.14-11.73 (m, 1H), 6.34 (s, 1H), 5.61-5.48 (m, 2H), 5.45-5.29 (m, 2H), 4.88-4.71 (m, 1H), 4.24-4.05 (m, 12H), 3.90 (s, 2H), 3.76-3.66 (m, 2H), 3.35-3.22 (m, 2H), 2.89 (d, J=5.6 Hz, 2H), 2.46-2.23 (m, 18H), 2.14-2.05 (m, 6H), 1.95 (s, 6H), 1.68 (s, 10H), 1.58-1.45 (m, 2H), 1.40-1.27 (m, 20H), 1.21 (s, 3H), 0.92 (d, J=5.4 Hz, 9H).

### Example 91—Compound 93

### O6-[2-[(4-fluorobicyclo[2.2.2]octane-1-carbonyl)oxymethyl]-2-[[6-[(Z)-non-3-enoxy]-6-oxo-hexanoyl]oxy methyl]-3-[4-(2-pyrrolidin-1-ylethylcarbamoyloxy)decanoyloxy]propyl]O1-[(Z)-non-3-enyl]hexanedioate

### Synthetic Scheme:

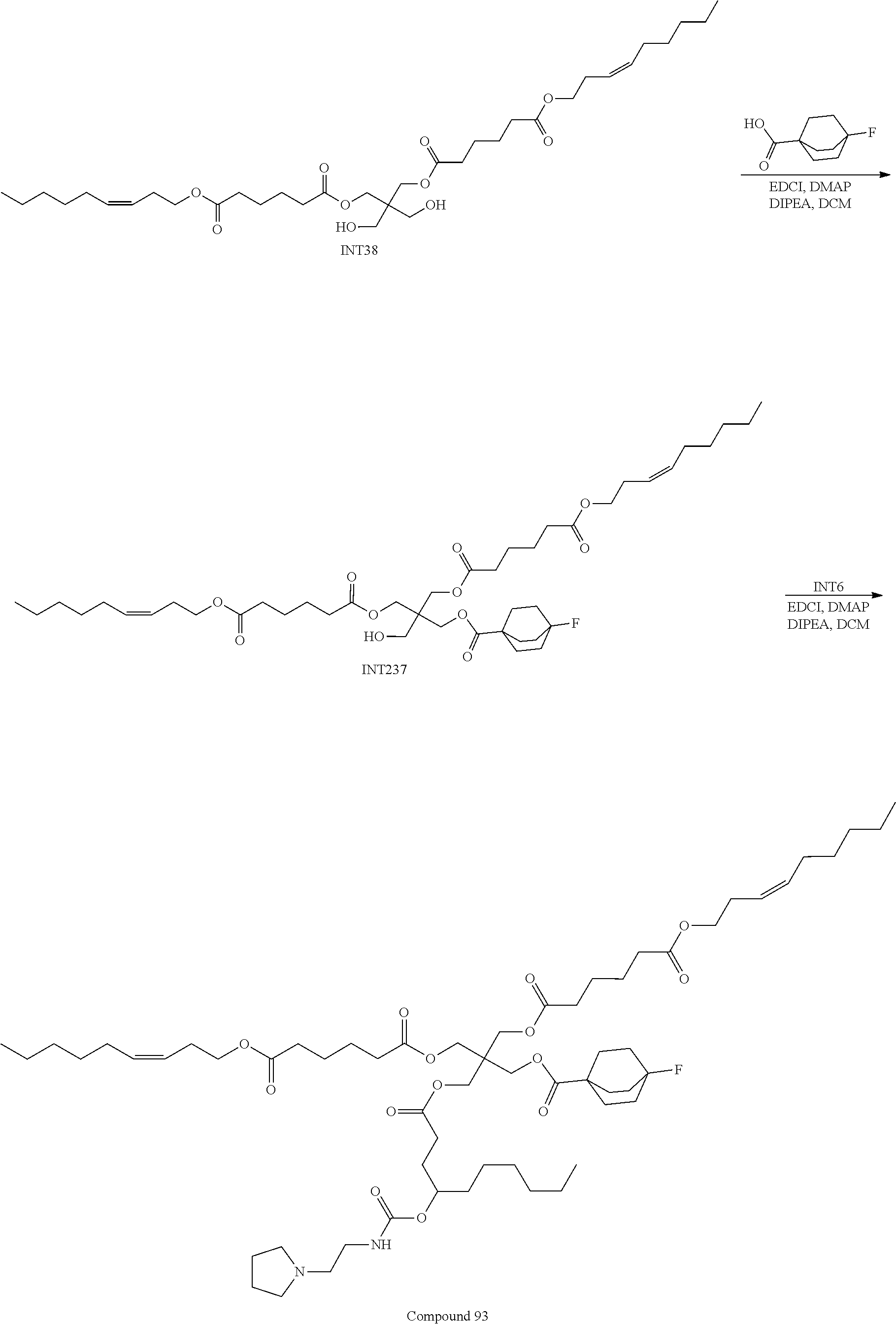

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (19 mL) was added EDCI (538.45 mg, 2.81 mmol, 1.2 eq), DMAP (28.60 mg, 234.07 mol, 0.1 eq), 4-fluorobicyclo[2.2.2]octane-1-carboxylic acid (403.05 mg, 2.34 mmol, 1 eq) and DIPEA (756.28 mg, 5.85 mmol, 1.02 mL, 2.5 eq) under N₂ atmosphere. The reaction mixture was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(4-fluorobicyclo[2.2.2]octane-1-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1 g, 1.26 mmol, 53.74% yield) was obtained as a colorless oil. ¹H NMR (400 MHz, CDCl₃) δ ppm 5.67-5.10 (m, 4H), 4.16-4.02 (m, 10H), 3.50 (s, 2H), 2.68-2.48 (m, 1H), 2.42-2.29 (m, 12H), 2.19-1.91 (m, 10H), 1.90-1.79 (m, 6H), 1.70-1.62 (m, 8H), 1.41-1.25 (m, 12H), 0.90 (t, J=6.8 Hz, 6H).

### Step 2:

To a solution of O6-[2-[(4-fluorobicyclo[2.2.2]octane-1-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1 g, 1.26 mmol, 1 eq) in DCM (15 mL) was added EDCI (289.35 mg, 1.51 mmol, 1.2 eq), DMAP (15.37 mg, 125.78 mol, 0.1 eq), 4-(2-pyrrolidin-1-ylethylcarbamoyloxy)decanoic acid (495.75 mg, 1.51 mmol, 1.2 eq) and DIPEA (406.41 mg, 3.14 mmol, 547.72 L, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1) and prep-HPLC (column: X-Select CSH Phenyl-Hexyl 100*30 5u; mobile phase: [H2O (0.04% HCl)-ACN:THF=1:1]; gradient: 35%-75% B over 12.0 min) to get compound O6-[2-[(4-fluorobicyclo[2.2.2]octane-1-carbonyl)oxymethyl]-2-[[6-[(Z)-non-3-enoxy]-6-oxo-hexanoyl]oxy methyl]-3-[4-(2-pyrrolidin-1-ylethylcarbamoyloxy)decanoyloxy]propyl]O1-[(Z)-non-3-enyl]hexanedioate (0.16 g, 144.74 mol, 11.51% yield, HCl salt) as a colorless oil. ¹H NMR (400 MHz, CD₃OD) δ ppm 5.55-5.31 (m, 4H), 4.80-4.71 (m, 1H), 4.22-4.05 (m, 12H), 3.72 (d, J=2.4 Hz, 1H), 3.51-3.46 (m, 2H), 3.36-3.32 (m, 2H), 3.24-3.06 (m, 2H), 2.42-2.32 (m, 14H), 2.21-2.03 (m, 8H), 2.02-1.90 (m, 8H), 1.87-1.68 (m, 8H), 1.66-1.54 (m, 10H), 1.42-1.29 (m, 20H), 0.95-0.87 (m, 9H).

### Example 92—Compound 96

### O6-[2-[(1-cyclohexylcyclohexanecarbonyl)oxymethyl]-2-[[6-[(Z)-non-3-enoxy]-6-oxo-hexanoyl]oxymethyl]-3-[4-(2-pyrrolidin-1-ylethylcarbamoyl-oxy)decanoyloxy]propyl]O1-[(Z)-non-3-enyl]hexanedioate

### Synthetic Scheme:

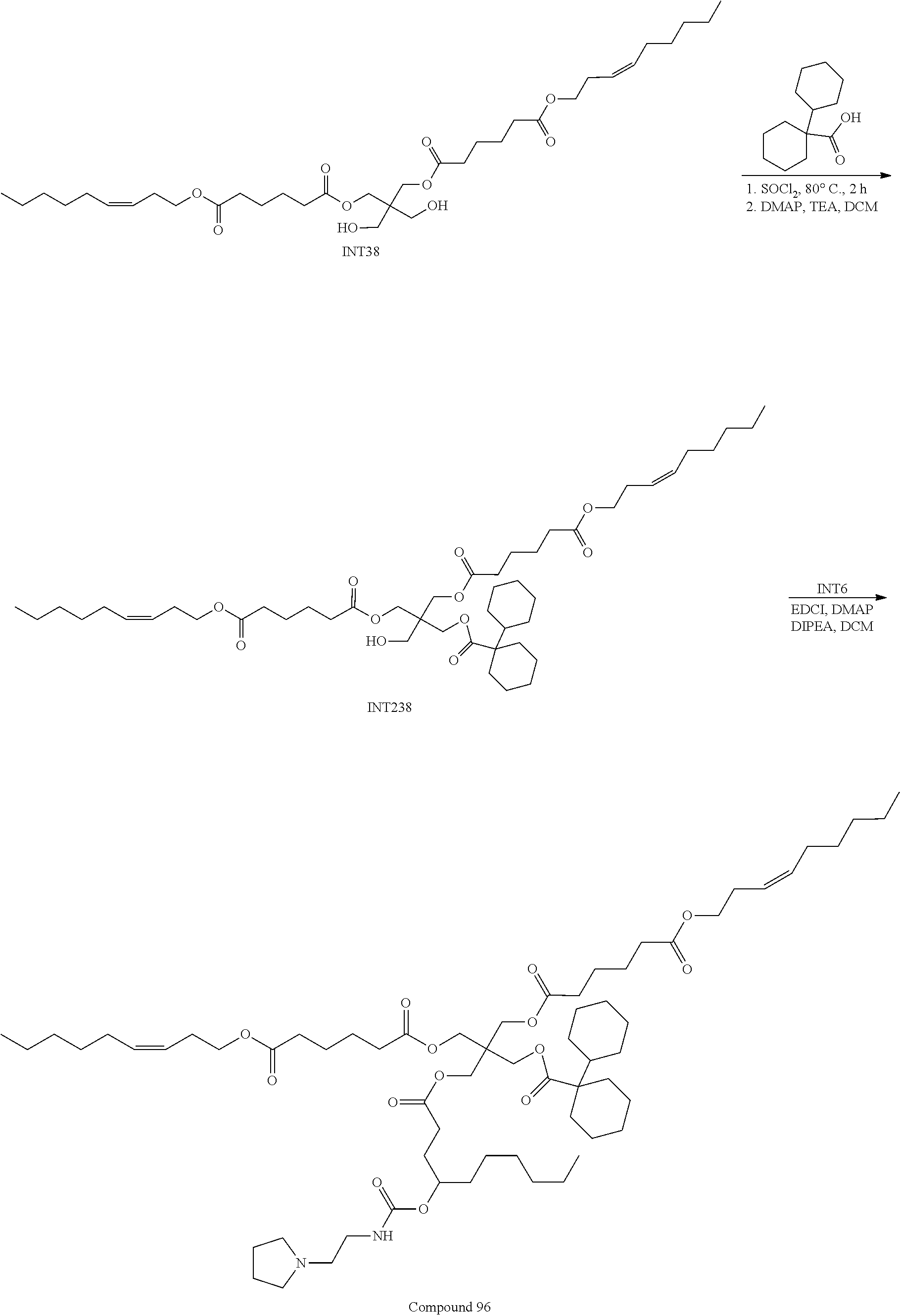

### Step 1:

A mixture of 1-cyclohexylcyclohexanecarboxylic acid (492.27 mg, 2.34 mmol, 3 eq) in SOCl₂ (464.12 mg, 3.90 mmol, 283.34 L, 5 eq) was stirred at 80° C. for 2 hr under N₂ atmosphere. The reaction mixture was concentrated under reduced pressure to get [1,1′-bi(cyclohexane)]-1-carbonyl chloride (500 mg, crude). To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (0.5 g, 780.22 mol, 1 eq), DMAP (9.53 mg, 78.02 mol, 0.1 eq) and TEA (394.75 mg, 3.90 mmol, 542.98 μL, 5 eq) in DCM (5 mL) was added [1,1′-bi(cyclohexane)]-1-carbonyl chloride (500 mg, crude) slowly under N₂ atmosphere. The reaction mixture was stirred at 25° C. for 12 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(1-cyclohexylcyclohexanecarbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.3 g, 360.08 mol, 23.08% yield) was obtained as a colorless oil.

### Step 2:

To a solution of O6-[2-[(1-cyclohexylcyclohexanecarbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.25 g, 300.07 mol, 1 eq) in DCM (2.5 mL) was added EDCI (69.03 mg, 360.08 mol, 1.2 eq), 4-(2-pyrrolidin-1-ylethylcarbamoyloxy)decanoic acid (118.27 mg, 360.08 mol, 1.2 eq), DIPEA (96.95 mg, 750.17 mol, 130.67 L, 2.5 eq) and DMAP (3.67 mg, 30.01 mol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(1-cyclohexylcyclohexanecarbonyl)oxymethyl]-2-[[6-[(Z)-non-3-enoxy]-6-oxo-hexanoyl]oxymethyl]-3-[4-(2-pyrrolidin-1-ylethylcarbamoyl-oxy)decanoyloxy]propyl]O1-[(Z)-non-3-enyl]hexanedioate (0.14 g, 122.42 mol, 40.80% yield) was obtained as a colorless oil. ¹H NMR (400 MHz, CDCl₃) δ ppm 5.59-5.46 (m, 2H), 5.40-5.28 (m, 2H), 5.25-5.10 (m, 1H), 4.84-4.69 (m, 1H), 4.17-4.04 (m, 12H), 3.30 (d, J=4.0 Hz, 2H), 2.71-2.44 (m, 6H), 2.42-2.30 (m, 14H), 2.08-2.00 (m, 6H), 1.95-1.86 (m, 1H), 1.78 (s, 6H), 1.72-1.45 (m, 18H), 1.43-1.10 (m, 30H), 0.91-0.86 (m, 9H).

### Example 93—Compound 97

### O6-[2-[[6-[(Z)-non-3-enoxy]-6-oxo-hexanoyl]oxymethyl]-2-[4-(2-pyrrolidin-1-ylethylcarbamoyloxy) decanoyloxymethyl]-3-(spiro[3.3]heptane-2-carbonyloxy)propyl]O1-[(Z)-non-3-enyl]hexanedioate

### Synthetic Scheme:

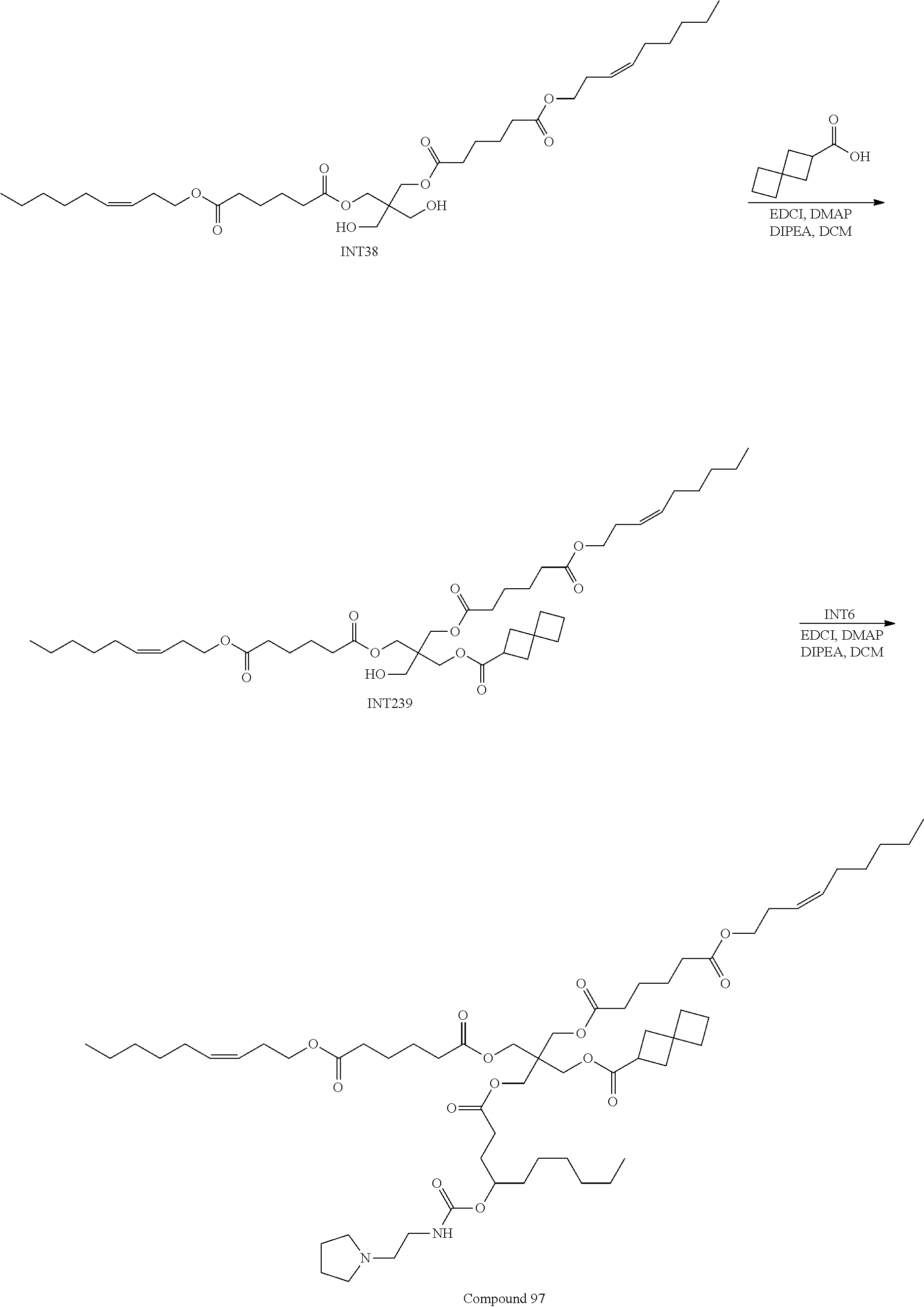

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (18 mL) was added EDCI (538.45 mg, 2.81 mmol, 1.2 eq), DMAP (28.60 mg, 234.07 mol, 0.1 eq), spiro[3.3]heptane-2-carboxylic acid (328.11 mg, 2.34 mmol, 1 eq) and DIPEA (756.28 mg, 5.85 mmol, 1.02 mL, 2.5 eq) under N₂ atmosphere. The reaction mixture was stirred at 25° C. for 10 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O6-[2-(hydroxymethyl)-2-[[6-[(Z)-non-3-enoxy]-6-oxo-hexanoyl]oxymethyl]-3-(spiro[3.3]heptane-2-carbonyloxy)propyl]O1-[(Z)-non-3-enyl]hexanedioate (0.8 g, 1.05 mmol, 44.79% yield) was obtained as a colorless oil. ¹H NMR (400 MHz, CDCl₃) δ ppm 5.54-5.30 (m, 4H), 4.11-4.05 (m, 10H), 3.50 (d, J=3.2 Hz, 2H), 3.04-2.93 (m, 1H), 2.62 (s, 1H), 2.41-2.31 (m, 12H), 2.22 (d, J=8.6 Hz, 4H), 2.08-2.01 (m, 6H), 1.95-1.90 (m, 2H), 1.84-1.76 (m, 2H), 1.69-1.63 (m, 8H), 1.38-1.26 (m, 12H), 0.94-0.84 (m, 6H).

### Step 2:

To a solution of O6-[2-(hydroxymethyl)-2-[[6-[(Z)-non-3-enoxy]-6-oxo-hexanoyl]oxymethyl]-3-(spiro[3.3]heptane-2-carbonyloxy)propyl]O1-[(Z)-non-3-enyl]hexanedioate (0.8 g, 1.05 mmol, 1 eq) in DCM (12 mL) was added EDCI (241.19 mg, 1.26 mmol, 1.2 eq), DMAP (12.81 mg, 104.85 mol, 0.1 eq), 4-(2-pyrrolidin-1-ylethylcarbamoyloxy)decanoic acid (413.24 mg, 1.26 mmol, 1.2 eq) and DIPEA (338.77 mg, 2.62 mmol, 456.57 L, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1). Compound O6-[2-[[6-[(Z)-non-3-enoxy]-6-oxo-hexanoyl]oxymethyl]-2-[4-(2-pyrrolidin-1-ylethylcarbamoyloxy) decanoyloxymethyl]-3-(spiro[3.3]heptane-2-carbonyloxy)propyl]O1-[(Z)-non-3-enyl]hexanedioate (0.28 g, 260.84 mol, 24.88% yield) was obtained as a colorless oil. ¹H NMR (400 MHz, CDCl₃) δ ppm 5.58-5.26 (m, 4H), 5.18 (s, 1H), 4.75 (d, J=2.6 Hz, 1H), 4.18-3.97 (m, 12H), 3.29 (d, J=4.6 Hz, 2H), 2.97 (t, J=8.6 Hz, 1H), 2.66-2.44 (m, 6H), 2.41-2.31 (m, 12H), 2.20 (d, J=8.4 Hz, 4H), 2.09-1.95 (m, 6H), 1.94-1.71 (m, 10H), 1.70-1.42 (m, 12H), 1.41-1.22 (m, 20H), 0.93-0.84 (m, 9H).

### Example 94—Compound 98

### O6-[2-(decalin-1-carbonyloxymethyl)-2-[[6-[(Z)-non-3-enoxy]-6-oxo-hexanoyl]oxymethyl]-3-[4-(2-pyrrolidin-1-ylethylcarbamoyloxy) decanoyloxy]propyl]O1-[(Z)-non-3-enyl]hexanedioate

### Synthetic Scheme:

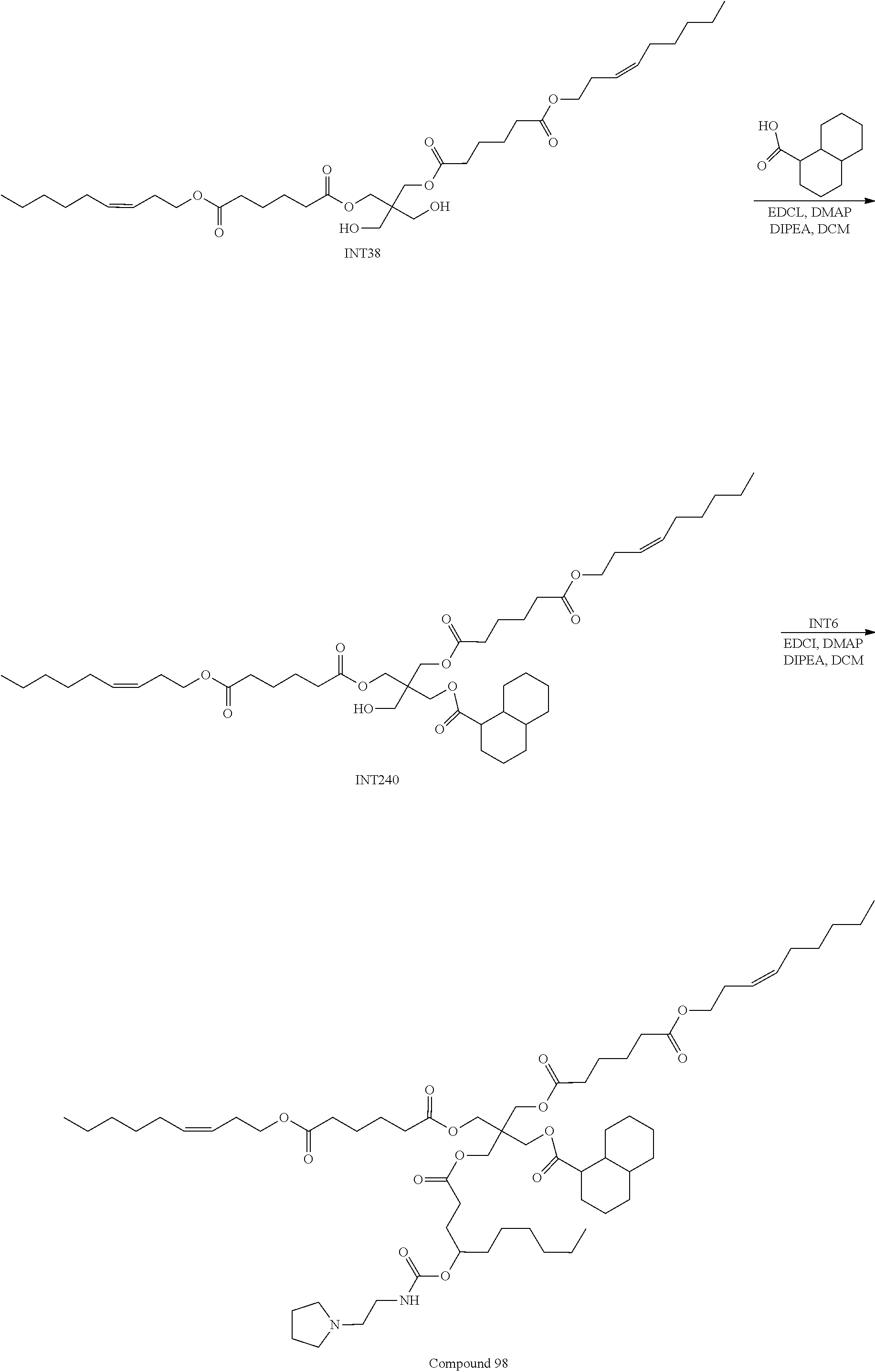

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (3 g, 4.68 mmol, 1 eq) in DCM (35 mL) was added EDCI (1.08 g, 5.62 mmol, 1.2 eq), DMAP (57.19 mg, 468.13 mol, 0.1 eq), decalin-1-carboxylic acid (511.93 mg, 2.81 mmol, 0.6 eq) and DIPEA (1.51 g, 11.70 mmol, 2.04 mL, 2.5 eq) under N₂ atmosphere. The reaction mixture was stirred at 25° C. for 10 hr under N₂ atmosphere. The reaction mixture was diluted with H₂O (100 mL) and extracted with DCM 200 mL (50 mL*4). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 1/1). Compound O6-[2-(decalin-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1.2 g, 1.49 mmol, 31.84% yield) was obtained as a colorless oil. ¹H NMR (400 MHz, CDCl₃) δ ppm 5.56-5.29 (m, 4H), 4.17-4.11 (m, 4H), 4.10-4.04 (m, 6H), 3.52 (s, 2H), 2.52-2.46 (m, 1H), 2.41-2.32 (m, 12H), 2.10-2.00 (m, 5H), 1.86-1.79 (m, 1H), 1.75-1.63 (m, 12H), 1.61-1.44 (m, 6H), 1.43-1.23 (m, 16H), 1.08-1.00 (m, 1H), 0.90 (t, J=6.8 Hz, 6H).

### Step 2:

To a solution of O6-[2-(decalin-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1.2 g, 1.49 mmol, 1 eq) in DCM (17 mL) was added EDCI (342.88 mg, 1.79 mmol, 1.2 eq), DMAP (18.21 mg, 149.05 mol, 0.1 eq), 4-(2-pyrrolidin-1-ylethylcarbamoyloxy)decanoic acid (587.47 mg, 1.79 mmol, 1.2 eq) and DIPEA (481.60 mg, 3.73 mmol, 649.05 L, 2.5 eq) under N₂ atmosphere. The reaction mixture was stirred at 25° C. for 10 hr under N₂ atmosphere. The reaction mixture was diluted with H₂O (100 mL) and extracted with DCM 150 mL (50 mL*3). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(decalin-1-carbonyloxymethyl)-2-[[6-[(Z)-non-3-enoxy]-6-oxo-hexanoyl]oxymethyl]-3-[4-(2-pyrrolidin-1-ylethylcarbamoyloxy) decanoyloxy]propyl]O1-[(Z)-non-3-enyl]hexanedioate (0.15 g, 134.47 mol, 9.02% yield, 100% purity) was obtained as a pale yellow oil. ¹H NMR (400 MHz, CDCl₃) δ ppm 5.62-5.26 (m, 4H), 4.77-4.68 (m, 1H), 4.24-4.01 (m, 12H), 3.27 (t, J=6.8 Hz, 2H), 2.64-2.58 (m, 5H), 2.56-2.50 (m, 1H), 2.44-2.28 (m, 14H), 2.13-2.04 (m, 4H), 1.97-1.88 (m, 1H), 1.84-1.68 (m, 8H), 1.67-1.45 (m, 16H), 1.44-1.16 (m, 26H), 1.07-1.01 (m, 1H), 0.94-0.88 (m, 9H).

### Example 95—Compound 99

### O6-[2-[[6-[(Z)-non-3-enoxy]-6-oxo-hexanoyl]oxymethyl]-2-(norbornane-1-carbonyloxymethyl)-3-[4-(2-pyrrolidin-1-ylethylcarbamoyloxy)decanoyloxy]propyl]O1-[(Z)-non-3-enyl]hexanedioate

### Synthetic Scheme:

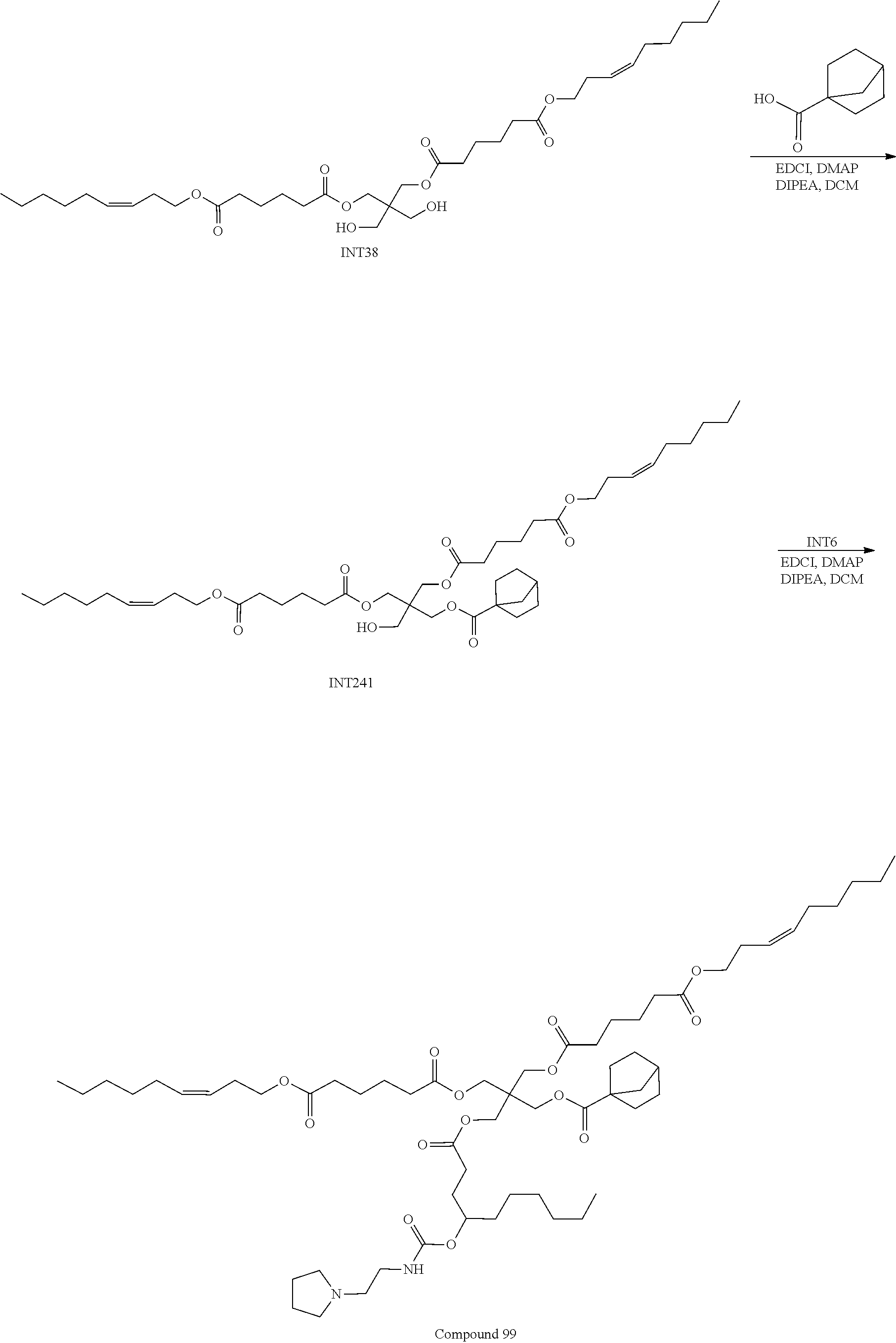

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq), norbornane-1-carboxylic acid (196.87 mg, 1.40 mmol, 0.6 eq), EDCI (538.45 mg, 2.81 mmol, 1.2 eq), DMAP (28.60 mg, 234.07 mol, 0.1 eq) and DIPEA (756.28 mg, 5.85 mmol, 1.02 mL, 2.5 eq) in DCM (15 mL) was stirred at 25° C. for 12 hours under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[[6-[(Z)-non-3-enoxy]-6-oxo-hexanoyl]oxymethyl]-3-(norbornane-1-carbonyloxy)propyl]O1-[(Z)-non-3-enyl]hexanedioate (0.85 g, 1.11 mmol, 47.59% yield) was obtained as a colorless oil. ¹H NMR (400 MHz, CDCl₃) δ ppm 5.55-5.47 (m, 2H), 5.38-5.30 (m, 2H), 4.15-4.06 (m, 10H), 3.51 (s, 2H), 2.63 (s, 1H), 2.39-2.31 (m, 12H), 2.07-2.01 (m, 4H), 1.90-1.83 (m, 2H), 1.68-1.64 (m, 8H), 1.59-1.52 (m, 6H), 1.39-1.28 (m, 14H), 0.90 (t, J=6.8 Hz, 6H).

### Step 2:

A mixture of O6-[2-(hydroxymethyl)-2-[[6-[(Z)-non-3-enoxy]-6-oxo-hexanoyl]oxymethyl]-3-(norbornane-1-carbonyloxy)propyl]O1-[(Z)-non-3-enyl]hexanedioate (0.85 g, 1.11 mmol, 1 eq), 4-(2-pyrrolidin-1-ylethylcarbamoyloxy)decanoic acid (439.07 mg, 1.34 mmol, 1.2 eq), DMAP (13.61 mg, 111.40 mol, 0.1 eq), EDCI (256.27 mg, 1.34 mmol, 1.2 eq) and DIPEA (359.94 mg, 2.79 mmol, 485.10 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: Xselect CSH C18 100*30 mm*5 um; mobile phase: [H₂O (0.04% HCl)-ACN:THF=1:1]; gradient: 40%-80% B over 12.0 min) to get compound O6-[2-[[6-[(Z)-non-3-enoxy]-6-oxo-hexanoyl]oxymethyl]-2-(norbornane-1-carbonyloxymethyl)-3-[4-(2-pyrrolidin-1-ylethylcarbamoyloxy)decanoyloxy]propyl]O1-[(Z)-non-3-enyl]hexanedioate (0.15 g, 139.74 mol, 12.54% yield, 100% purity, HCl salt) was obtained as a colorless oil. ¹H NMR (400 MHz, CDCl₃) δ ppm 12.79-12.00 (m, 1H), 6.30 (t, J=5.8 Hz, 1H), 5.55-5.47 (m, 2H), 5.38-5.31 (m, 2H), 4.81-4.67 (m, 1H), 4.17-4.04 (m, 12H), 3.90-3.83 (m, 2H), 3.72- 3.62 (m, 2H), 3.31-3.20 (m, 2H), 2.93-2.80 (m, 2H), 2.39-2.31 (m, 14H), 2.28-2.21 (m, 2H), 2.12-2.01 (m, 6H), 1.91-1.68 (m, 6H), 1.67-1.63 (m, 8H), 1.59-1.55 (m, 6H), 1.37-1.25 (m, 22H), 0.92-0.86 (m, 9H).

### Example 96—Compound 100

### O6-[2-[(2,2-difluorospiro[3.3]heptane-6-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

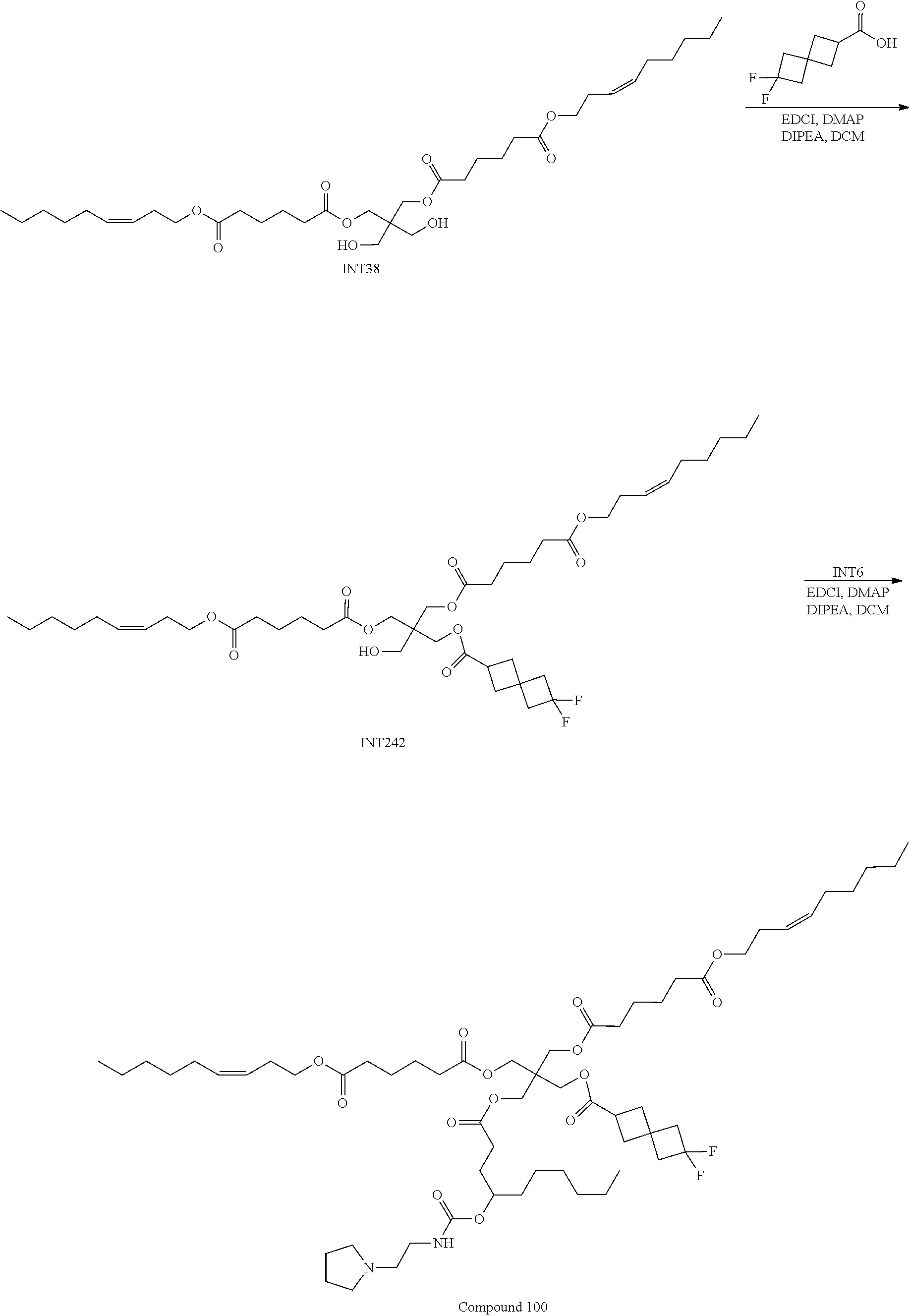

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added 2,2-difluorospiro[3.3]heptane-6-carboxylic acid (412.33 mg, 2.34 mmol, 1 eq), DIPEA (756.28 mg, 5.85 mmol, 1.02 mL, 2.5 eq), EDCI (538.45 mg, 2.81 mmol, 1.2 eq) and DMAP (28.60 mg, 234.07 mol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(2,2-difluorospiro[3.3]heptane-6-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1.2 g, 1.50 mmol, 64.17% yield) was obtained as a colorless oil.

### Step 2:

To a solution of O6-[2-[(2,2-difluorospiro[3.3]heptane-6-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (1 g, 1.25 mmol, 1 eq) in DCM (10 mL) was added EDCI (287.92 mg, 1.50 mmol, 1.2 eq), DIPEA (485.27 mg, 3.75 mmol, 654.01 μL, 3 eq), 4-(2-pyrrolidin-1-ylethylcarbamoyloxy)decanoic acid (493.29 mg, 1.50 mmol, 1.2 eq) and DMAP (18.55 mg, 125.16 mol, 0.1 eq) under N₂ atmosphere. The reaction mixture was stirred at 25° C. for 12 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 35%-75% B over 12.0 min) to get compound O6-[2-[(2,2-difluorospiro[3.3]heptane-6-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.16 g, 144.22 mol, 11.52% yield, HCl salt) as a colorless oil. ¹H NMR (400 MHz, CD₃OD) δ ppm 5.57-5.45 (m, 2H), 5.41-5.31 (m, 2H), 4.81-4.71 (m, 1H), 4.20-4.04 (m, 12H), 3.72 (d, J=5.4 Hz, 2H), 3.53-3.44 (m, 2H), 3.36-3.33 (m, 2H), 3.19-3.13 (m, 2H), 2.69-2.49 (m, 4H), 2.42-2.31 (m, 18H), 2.24-2.13 (m, 2H), 2.10-2.01 (m, 6H), 1.97-1.73 (m, 2H), 1.68-1.55 (m, 10H), 1.43-1.29 (m, 20H), 0.97-0.85 (m, 9H).

### Example 97—Compound 101

### O6-[2-(bicyclo[3.1.0]hexane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

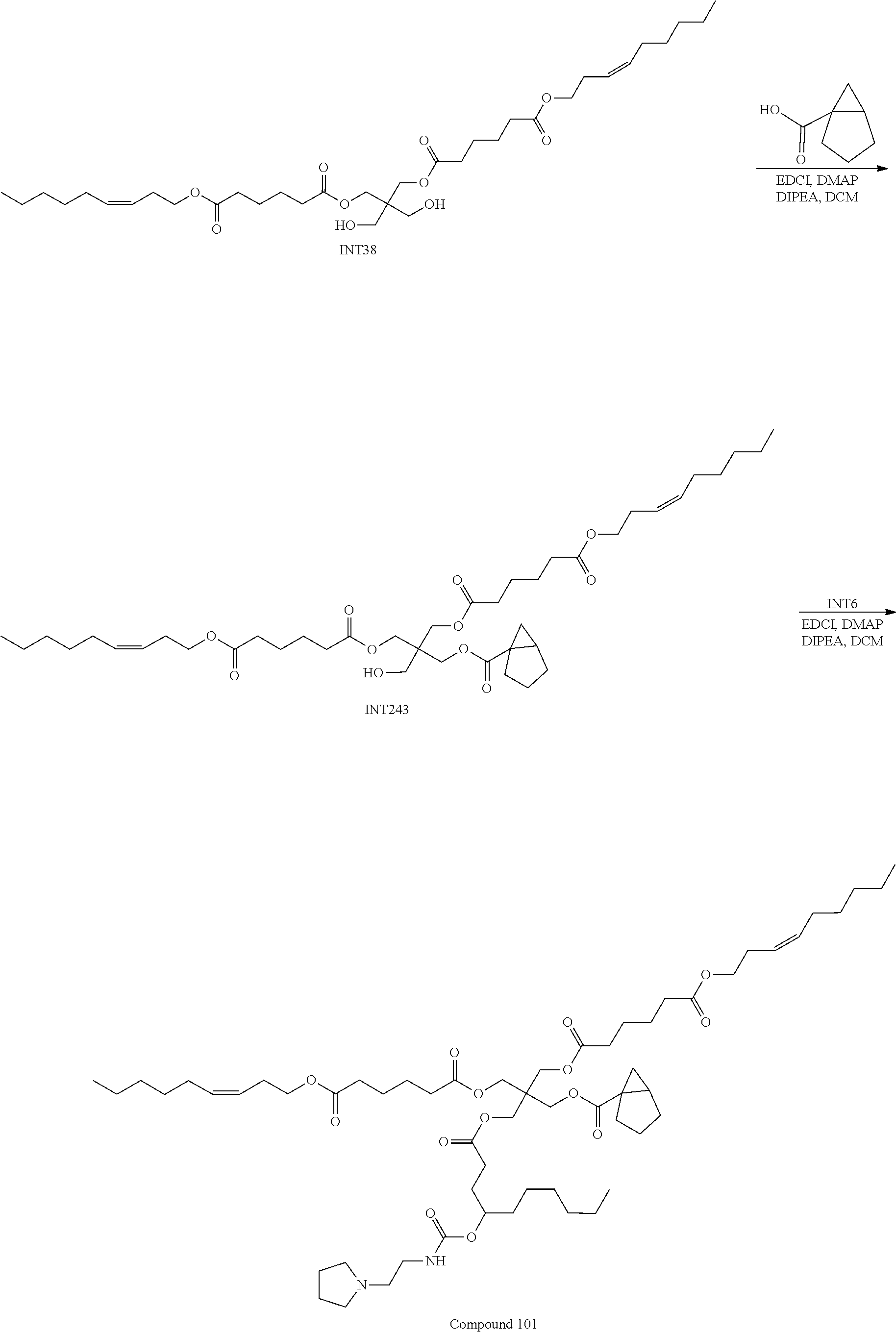

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq), bicyclo[3.1.0]hexane-1-carboxylic acid (177.17 mg, 1.40 mmol, 0.6 eq), EDCI (538.45 mg, 2.81 mmol, 1.2 eq), DMAP (28.60 mg, 234.07 mol, 0.1 eq) and DIPEA (756.28 mg, 5.85 mmol, 1.02 mL, 2.5 eq) in DCM (15 mL) was stirred at 25° C. for 12 h under N₂ atmosphere. The reaction mixture was diluted with H₂O (100 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[3.1.0]hexane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.13 mmol, 48.49% yield) was obtained as a colorless oil. ¹H NMR (400 MHz, CDCl₃) δ ppm 5.56-5.46 (m, 2H), 5.39-5.29 (m, 2H), 4.12-4.05 (m, 10H), 3.50 (d, J=6.2 Hz, 2H), 2.66 (t, J=6.6 Hz, 1H), 2.40-2.31 (m, 12H), 2.15-2.08 (m, 1H), 2.07-2.01 (m, 4H), 1.90-1.84 (m, 2H), 1.82-1.73 (m, 2H), 1.68-1.64 (m, 8H), 1.58 (s, 1H), 1.39-1.26 (m, 14H), 0.91-0.88 (m, 6H).

### Step 2:

A mixture of O6-[2-(bicyclo[3.1.0]hexane-1-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (0.85 g, 1.13 mmol, 1 eq), 4-(2-pyrrolidin-1-ylethylcarbamoyloxy)decanoic acid (447.29 mg, 1.36 mmol, 1.2 eq), EDCI (261.07 mg, 1.36 mmol, 1.2 eq), DMAP (13.86 mg, 113.49 mol, 0.1 eq) and DIPEA (366.69 mg, 2.84 mmol, 494.19 L, 2.5 eq) in DCM (8.5 mL) was stirred at 25° C. for 12 h under N₂ atmosphere. The reaction mixture was diluted with H₂O (50 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: Phenomenex Luna C18 100*30 mm*3 um; mobile phase: [H₂O (0.04% HCl)-ACN:THF=1:1]; gradient: 40%-80% B over 12.0 min) to get compound O6-[2-(bicyclo[3.1.0]hexane-1-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 141.59 mol, 12.48% yield, 100% purity, HCl salt) as a colorless oil. ¹H NMR (400 MHz, CDCl₃) δ ppm 12.53-12.23 (m, 1H), 6.42-6.17 (m, 1H), 5.55-5.47 (m, 2H), 5.38-5.30 (m, 2H), 4.78-4.70 (m, 1H), 4.19-4.09 (m, 8H), 4.09-4.05 (m, 4H), 3.90-3.83 (m, 2H), 3.71-3.61 (m, 2H), 3.29-3.20 (m, 2H), 2.91-2.81 (m, 2H), 2.39-2.30 (m, 14H), 2.28-2.22 (m, 2H), 2.10-2.01 (m, 6H), 1.88-1.74 (m, 6H), 1.67-1.63 (m, 8H), 1.57-1.45 (m, 2H), 1.40-1.18 (m, 24H), 0.91-0.86 (m, 9H).

### Example 98—Compound 102

### O6-[2-(decalin-2-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

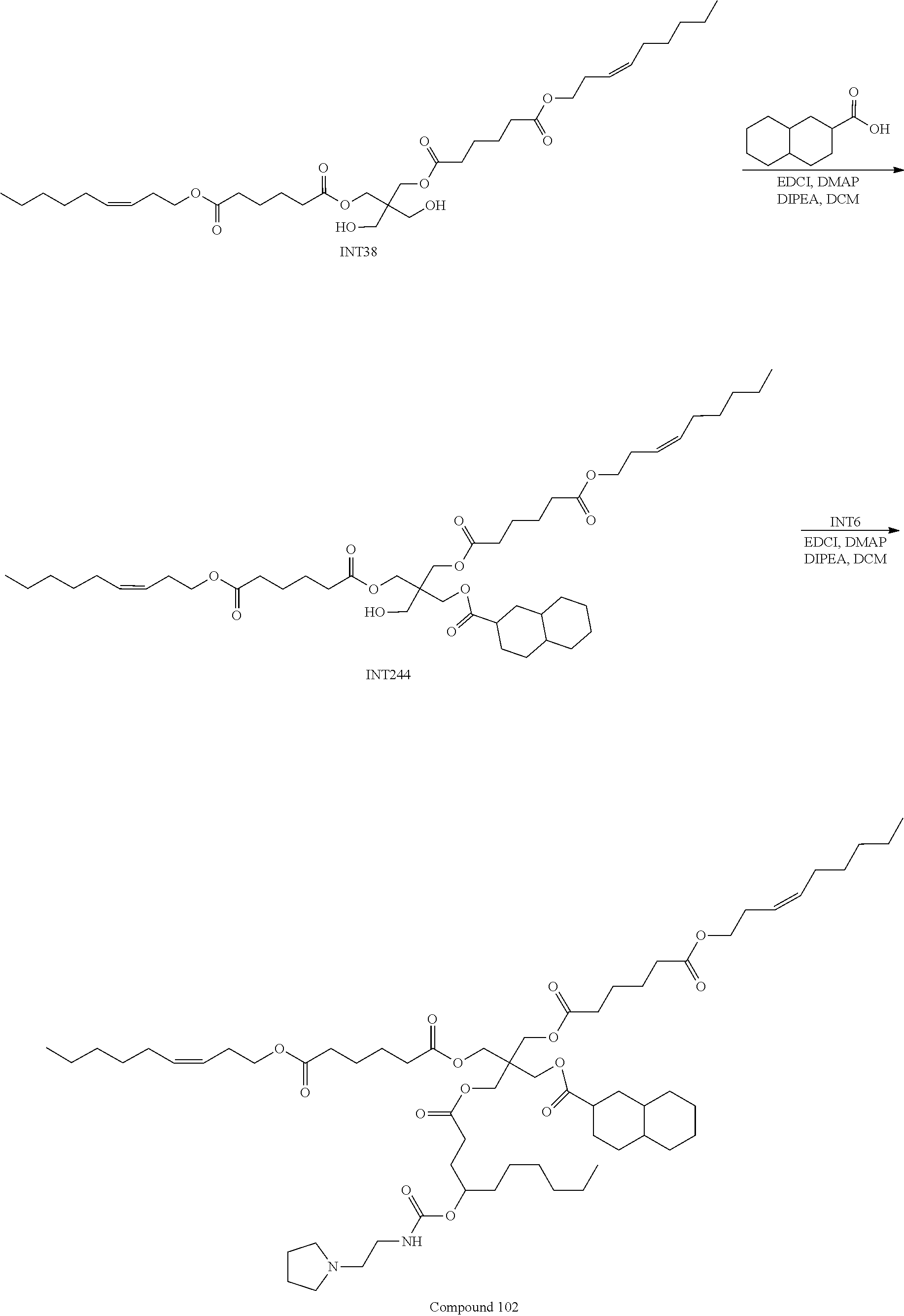

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq) in DCM (20 mL) was added EDCI (717.93 mg, 3.75 mmol, 1.2 eq), DIPEA (1.01 g, 7.80 mmol, 1.36 mL, 2.5 eq), decalin-2-carboxylic acid (455.05 mg, 2.50 mmol, 0.8 eq) and DMAP (38.13 mg, 312.09 mol, 0.1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(decalin-2-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.24 mmol, 39.80% yield) was obtained as a colorless oil.

### Step 2:

To a solution of O6-[2-(decalin-2-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.24 mmol, 1 eq) in DCM (10 mL) was added EDCI (285.73 mg, 1.49 mmol, 1.2 eq), DIPEA (401.33 mg, 3.11 mmol, 540.88 L, 2.5 eq), 4-(2-pyrrolidin-1-ylethylcarbamoyloxy)decanoic acid (489.56 mg, 1.49 mmol, 1.2 eq) and DMAP (15.17 mg, 124.21 mol, 0.1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(decalin-2-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.16 g, 143.43 mol, 11.55% yield) was obtained as a colorless oil. ¹H NMR (400 MHz, CDCl₃) δ ppm 5.58-5.46 (m, 2H), 5.42-5.28 (m, 2H), 5.18 (d, J=5.4 Hz, 1H), 4.75 (s, 1H), 4.15-4.04 (m, 12H), 3.30 (d, J=3.2 Hz, 2H), 2.68-2.49 (m, 6H), 2.43-2.31 (m, 14H), 2.07-2.01 (m, 4H), 1.96-1.87 (m, 2H), 1.82-1.74 (m, 6H), 1.72-1.58 (m, 14H), 1.54-1.47 (m, 4H), 1.40-1.21 (m, 26H), 0.93-0.85 (m, 9H).

### Example 99—Compound 103

### O6-[2-[(7,7-difluoronorcarane-3-carbonyl)oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate

### Synthetic Scheme:

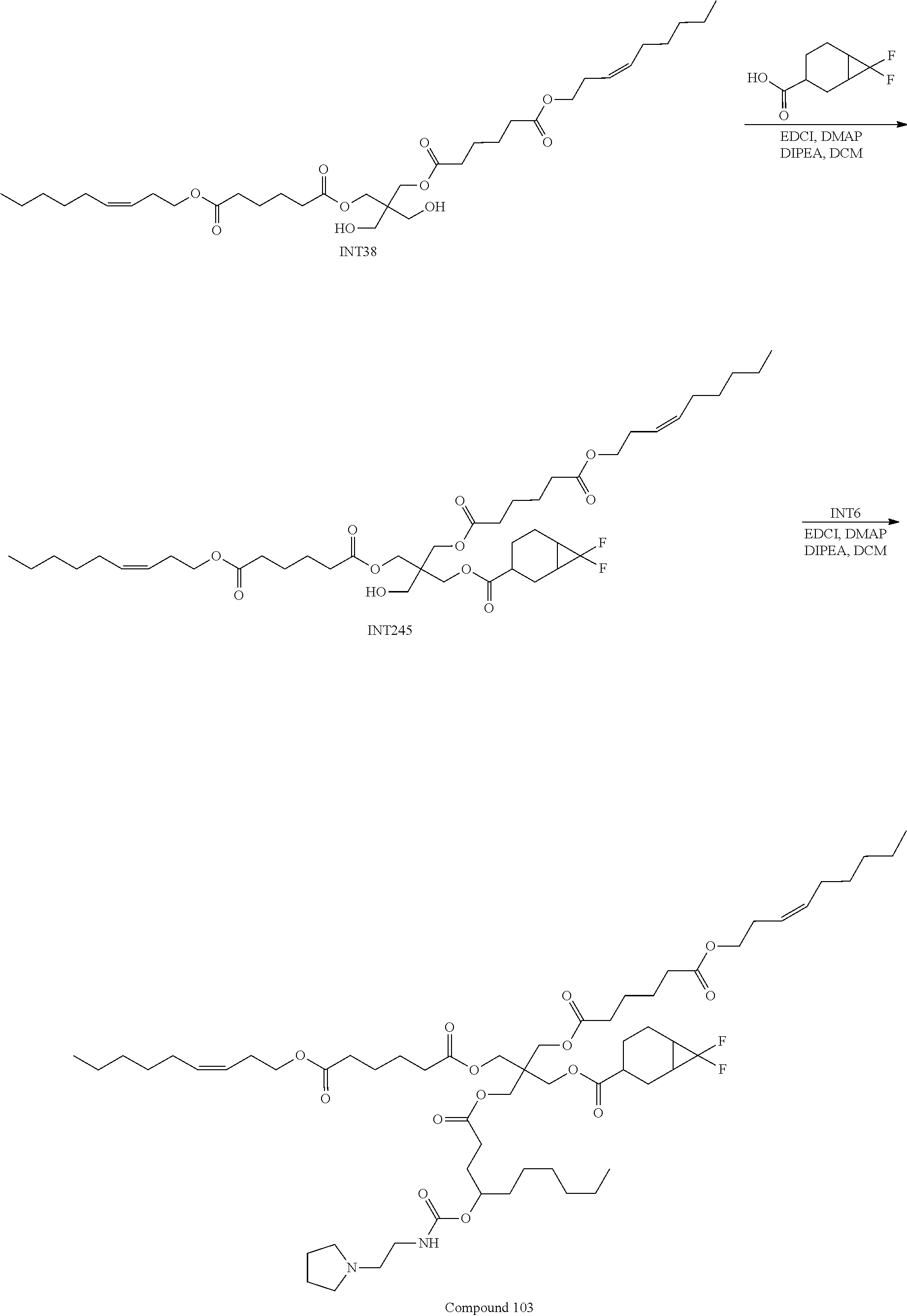

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq), 7,7-difluoronorcarane-3-carboxylic acid (247.40 mg, 1.40 mmol, 0.6 eq), DMAP (28.60 mg, 234.07 mol, 0.1 eq), EDCI (538.45 mg, 2.81 mmol, 1.2 eq) and DIPEA (756.28 mg, 5.85 mmol, 1.02 mL, 2.5 eq) in DCM (15 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(7,7-difluoronorcarane-3-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.06 mmol, 45.45% yield) was obtained as a colorless oil. ¹H NMR (400 MHz, CDCl₃) δ ppm 5.48-5.39 (m, 2H), 5.30-5.24 (m, 2H), 4.05-3.97 (m, 10H), 3.44 (s, 2H), 2.32-2.24 (m, 12H), 2.00-1.89 (m, 6H), 1.61-1.55 (m, 10H), 1.47 (s, 6H), 1.30-1.20 (m, 12H), 0.82 (t, J=6.8 Hz, 6H).

### Step 2:

A mixture of O6-[2-[(7,7-difluoronorcarane-3-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.06 mmol, 1 eq), 4-(2-pyrrolidin-1-ylethylcarbamoyloxy)decanoic acid (419.30 mg, 1.28 mmol, 1.2 eq), EDCI (244.73 mg, 1.28 mmol, 1.2 eq), DMAP (13.00 mg, 106.38 mol, 0.1 eq) and DIPEA (343.74 mg, 2.66 mmol, 463.25 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 35%-75% B over 12.0 min) to get compound O6-[2-[(7,7-difluoronorcarane-3-carbonyl)oxymethyl]-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate (0.15 g, 135.21 mol, 12.71% yield, 100% purity, HCl salt) was obtained as a colorless oil. ¹H NMR (400 MHz, CDCl₃) δ ppm 12.83-11.74 (m, 1H), 6.73-6.08 (m, 1H), 5.56-5.46 (m, 2H), 5.38-5.30 (m, 2H), 4.80-4.67 (m, 1H), 4.17-4.04 (m, 12H), 3.87 (d, J=5.6 Hz, 2H), 3.72-3.60 (m, 2H), 3.31-3.19 (m, 2H), 2.91-2.79 (m, 2H), 2.39-2.31 (m, 14H), 2.28-2.22 (m, 2H), 2.12-1.98 (m, 8H), 1.85 (s, 6H), 1.68-1.62 (m, 10H), 1.38-1.24 (m, 22H), 0.93-0.86 (m, 9H).

### Example 100—Compound 104

### O6-[2-[(2,2-difluorospiro[2.3]hexane-5-carbonyl)oxymethyl]-2-[[6-[(Z)-non-3-enoxy]-6-oxohexanoy]oxymethyl]-3-[4-(2-pyrrolidin-1-ylethylcarbamoyloxy)decanoyloxy]propyl]O1-[(Z)-non-3-enyl]hexanedioate

### Synthetic Scheme:

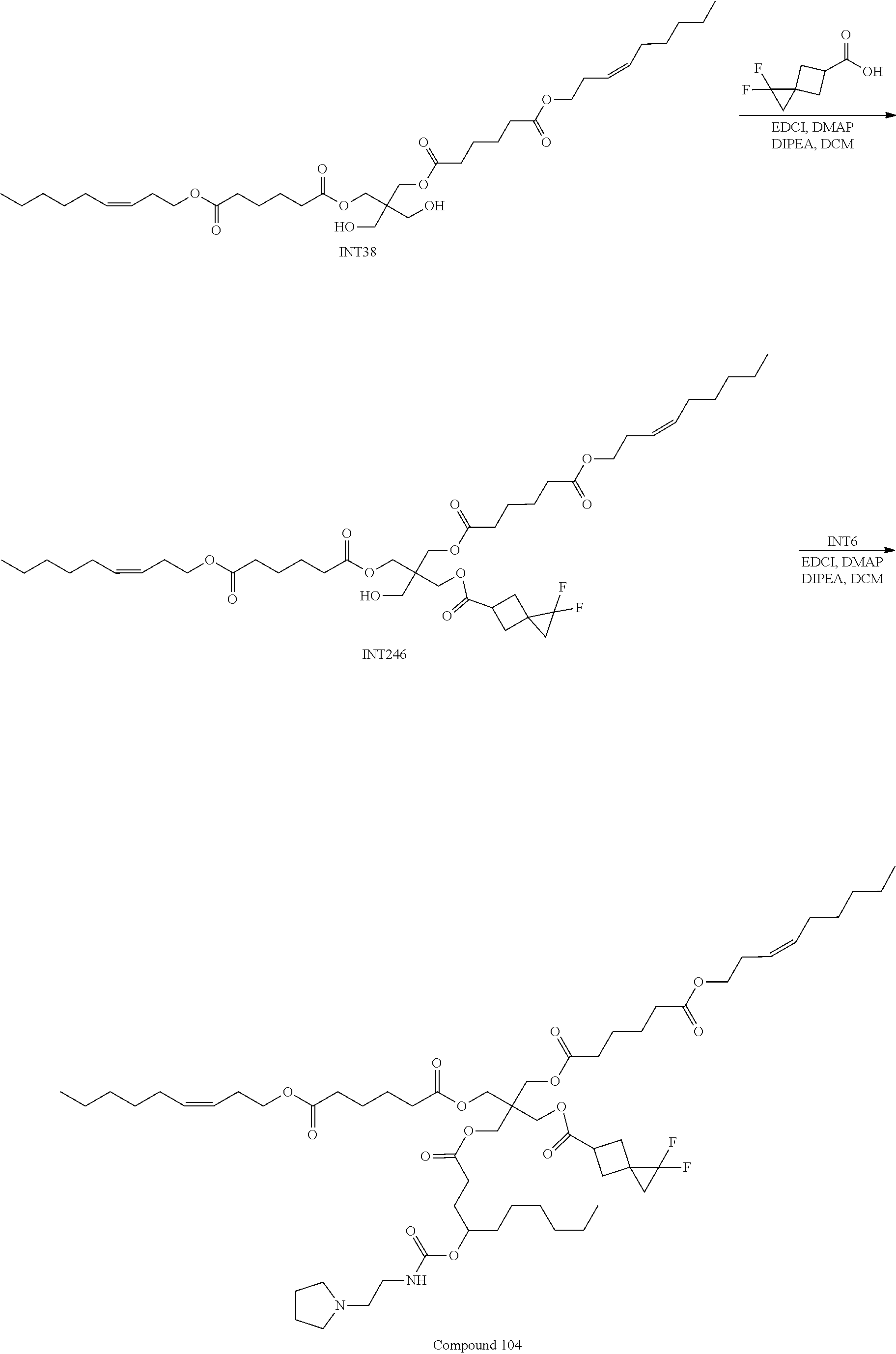

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (18 mL) was added EDCI (538.45 mg, 2.81 mmol, 1.2 eq), DMAP (28.60 mg, 234.07 mol, 0.1 eq), 2,2-difluorospiro[2.3]hexane-5-carboxylic acid (379.50 mg, 2.34 mmol, 1 eq) and DIPEA (756.28 mg, 5.85 mmol, 1.02 mL, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound O6-[2-[(2,2-difluorospiro[2.3]hexane-5-carbonyl)oxymethyl]-2-(hydroxymethyl)-3-[6-[(Z)-non-3-enoxy]-6-oxo-hexan-oyl]oxy-propyl]O1-[(Z)-non-3-enyl]hexanedioate (0.8 g, 1.02 mmol, 43.54% yield) was obtained as a colorless oil. ¹H NMR (400 MHz, CDCl₃) δ ppm 5.57-5.29 (m, 4H), 4.18-4.05 (m, 10H), 3.53 (s, 2H), 3.33-3.19 (m, 1H), 2.63-2.43 (m, 4H), 2.41-2.32 (m, 12H), 2.08-2.00 (m, 4H), 1.66 (t, J=3.0 Hz, 8H), 1.57 (s, 1H), 1.40-1.23 (m, 14H), 0.90 (t, J=6.8 Hz, 6H).

### Step 2:

To a solution of O6-[2-[(2,2-difluorospiro[2.3]hexane-5-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.8 g, 1.02 mmol, 1 eq) in DCM (12 mL) was added EDCI (234.45 mg, 1.22 mmol, 1.2 eq), DMAP (12.45 mg, 101.92 mol, 0.1 eq), 4-(2-pyrrolidin-1-ylethylcarbamoyloxy)decanoic acid (401.69 mg, 1.22 mmol, 1.2 eq) and DIPEA (329.30 mg, 2.55 mmol, 443.80 L, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 40%-75% B over 18.0 min) to get compound O6-[2-[(2,2-difluorospiro[2.3]hexane-5-carbonyl)oxymethyl]-2-[[6-[(Z)-non-3-enoxy]-6-oxohexanoy]oxymethyl]-3-[4-(2-pyrrolidin-1-ylethylcarbamoyloxy)decanoyloxy]propyl]O1-[(Z)-non-3-enyl]hexanedioate (0.3 g, 273.87 mol, 26.87% yield, HCl salt) as a colorless oil. ¹H NMR (400 MHz, CD₃OD) δ ppm 5.55-5.32 (m, 4H), 4.79 (d, J=9.6 Hz, 2H), 4.24-4.05 (m, 12H), 3.73 (s, 2H), 3.53-3.45 (m, 2H), 3.35-3.32 (m, 2H), 3.17-3.11 (m, 2H), 2.58-2.29 (m, 20H), 2.20-2.14 (m, 2H), 2.09-1.90 (m, 8H), 1.83-1.76 (m, 1H), 1.66-1.53 (m, 10H), 1.39-1.26 (m, 22H), 0.97-0.85 (m, 9H).

### Example 101—Compound 105

### O6-[2-[(5-methylbicyclo[3.3.1]nonane-1-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

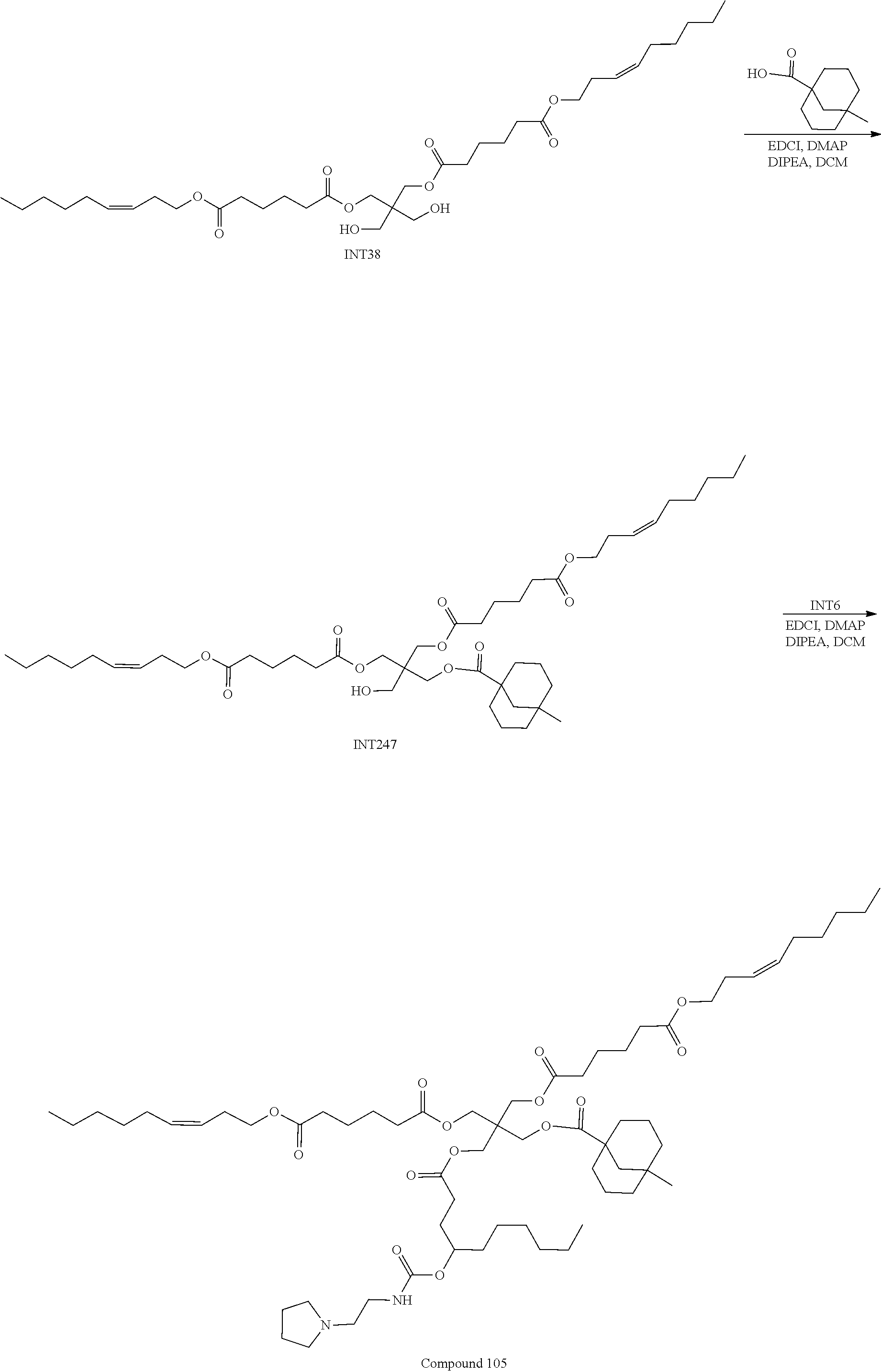

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 5-methylbicyclo[3.3.1]nonane-1-carboxylic acid (341.29 mg, 1.87 mmol, 0.6 eq), EDCI (717.93 mg, 3.75 mmol, 1.2 eq), DIPEA (1.01 g, 7.80 mmol, 1.36 mL, 2.5 eq) and DMAP (38.13 mg, 312.09 mol, 0.1 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[(5-methylbicyclo[3.3.1]nonane-1-carbonyl)oxymethyl]-3-[6-[(Z)-non-3-enoxy]-6-oxo-hexanoyl]oxy-propyl]O1-[(Z)-non-3-enyl]hexanedioate (0.85 g, 1.06 mmol, 33.83% yield) was obtained as a colorless oil. ¹H NMR (400 MHz, CDCl₃) δ ppm 5.55-5.47 (m, 2H), 5.38-5.30 (m, 2H), 4.14-4.05 (m, 10H), 3.51 (s, 2H), 2.63 (d, J=1.4 Hz, 1H), 2.39-2.30 (m, 12H), 2.07-2.02 (m, 4H), 1.92-1.86 (m, 2H), 1.70-1.61 (m, 12H), 1.58-1.52 (m, 3H), 1.46 (s, 2H), 1.38-1.24 (m, 16H), 0.92-0.88 (m, 9H).

### Step 2:

A mixture of O6-[2-(hydroxymethyl)-2-[(5-methylbicyclo[3.3.1]nonane-1-carbonyl)oxymethyl]-3-[6-[(Z)-non-3-enoxy]-6-oxo-hexanoyl]oxy-propyl]O1-[(Z)-non-3-enyl]hexanedioate (850.00 mg, 1.06 mmol, 1 eq), 4-(2-pyrrolidin-1-ylethylcarbamoyloxy)decanoic acid (416.12 mg, 1.27 mmol, 1.2 eq), EDCI (242.87 mg, 1.27 mmol, 1.2 eq), DMAP (12.90 mg, 105.58 mol, 0.1 eq) and DIPEA (341.13 mg, 2.64 mmol, 459.75 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(5-methylbicyclo[3.3.1]nonane-1-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 134.47 mol, 12.74% yield, 100% purity) was obtained as a colorless oil. ¹H NMR (400 MHz, CDCl₃) δ ppm 5.56-5.47 (m, 2H), 5.38-5.30 (m, 2H), 5.26-5.03 (m, 1H), 4.75 (d, J=3.0 Hz, 1H), 4.12 (s, 6H), 4.10-4.05 (m, 6H), 3.41-3.19 (m, 2H), 2.63-2.49 (m, 5H), 2.41-2.31 (m, 14H), 2.08-1.96 (m, 6H), 1.90- 1.84 (m, 3H), 1.79 (s, 4H), 1.67-1.64 (m, 8H), 1.61 (d, J=3.4 Hz, 2H), 1.59-1.56 (m, 4H), 1.53 (d, J=6.2 Hz, 2H), 1.44 (s, 2H), 1.38-1.23 (m, 24H), 0.92-0.87 (m, 12H).

### Example 102—Compound 106

### O6-[2-[[(1S,5R)-6,6-difluorobicyclo[3.1.0]hexane-3-carbonyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

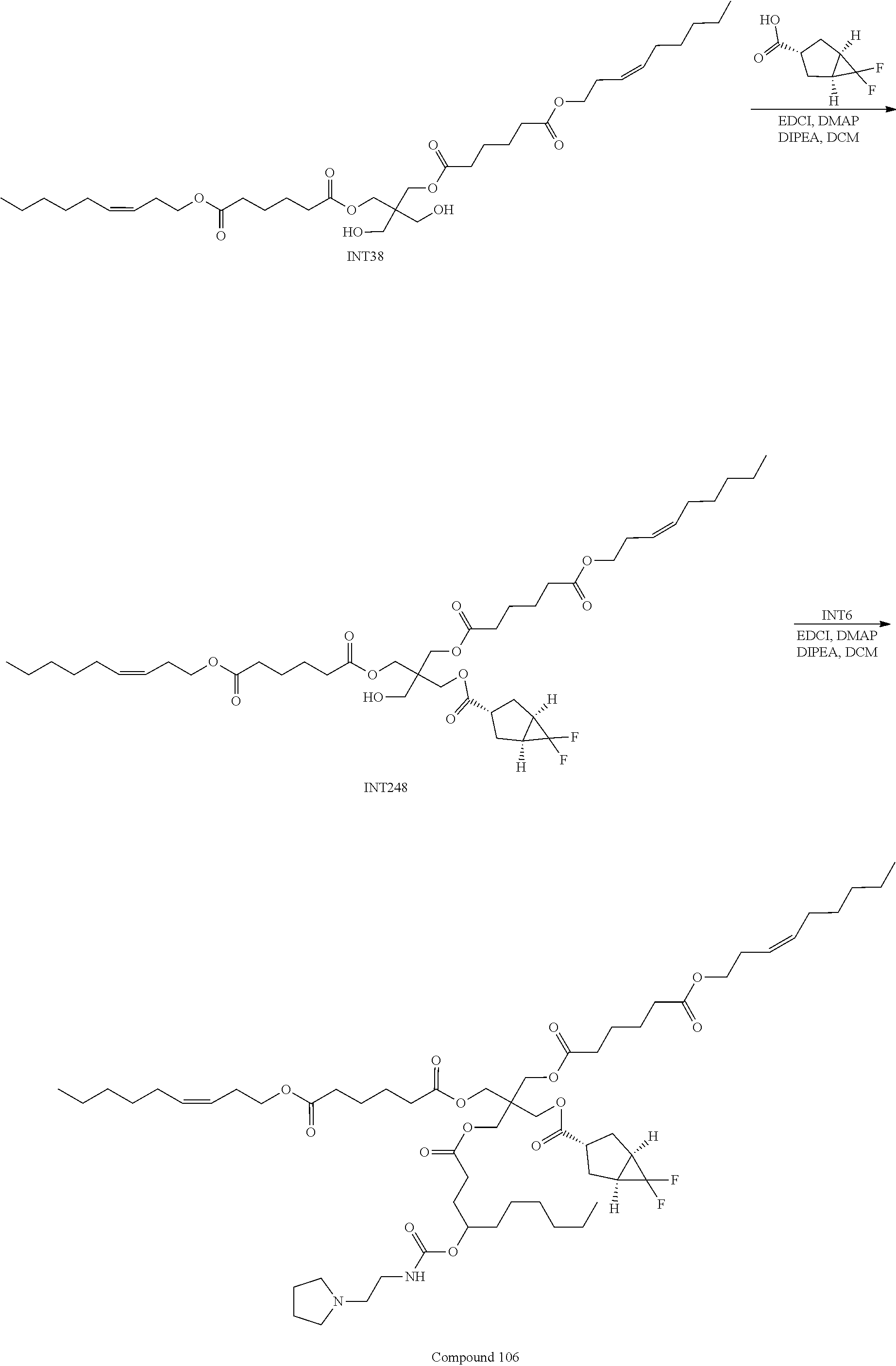

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), (1S,5R)-6,6-difluorobicyclo[3.1.0]hexane-3-carboxylic acid (404.80 mg, 2.50 mmol, 0.8 eq), DMAP (38.13 mg, 312.09 mol, 0.1 eq), EDCI (717.93 mg, 3.75 mmol, 1.2 eq) and DIPEA (1.01 g, 7.80 mmol, 1.36 mL, 2.5 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[(1S,5R)-6,6-difluorobicyclo[3.1.0]hexane-3-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.08 mmol, 34.70% yield) was obtained as a colorless oil. ¹H NMR (400 MHz, CDCl₃) δ ppm 5.48-5.39 (m, 2H), 5.31-5.22 (m, 2H), 4.05-3.98 (m, 10H), 3.43 (s, 2H), 2.83-2.73 (m, 1H), 2.33-2.22 (m, 14H), 2.19-2.12 (m, 2H), 2.00-1.92 (m, 6H), 1.61-1.56 (m, 8H), 1.49 (s, 2H), 1.30-1.20 (m, 12H), 0.82 (t, J=6.8 Hz, 6H).

### Step 2:

A mixture of O6-[2-[[(S,5R)-6,6-difluorobicyclo[3.1.0]hexane-3-carbonyl]oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.08 mmol, 1 eq), 4-(2-pyrrolidin-1-ylethylcarbamoyloxy)decanoic acid (426.79 mg, 1.30 mmol, 1.2 eq), EDCI (249.10 mg, 1.30 mmol, 1.2 eq), DMAP (13.23 mg, 108.29 mol, 0.1 eq) and DIPEA (349.88 mg, 2.71 mmol, 471.53 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 35%-75% B over 12.0 min) to get compound O6-[2-[[(1S,5R)-6,6-difluorobicyclo[3.1.0]hexane-3-carbonyl]oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 136.94 mol, 12.65% yield, 100% purity, HCl salt) as a colorless oil. ¹H NMR (400 MHz, CDCl₃) δ ppm 12.32 (s, 1H), 6.52-6.17 (m, 1H), 5.55-5.47 (m, 2H), 5.38-5.29 (m, 2H), 4.78-4.70 (m, 1H), 4.15-4.02 (m, 12H), 3.87 (d, J=4.6 Hz, 2H), 3.66 (d, J=6.4 Hz, 2H), 3.30-3.20 (m, 2H), 2.87-2.82 (m, 2H), 2.40-2.31 (m, 14H), 2.27-2.21 (m, 4H), 2.11-2.00 (m, 8H), 1.94-1.76 (m, 6H), 1.65 (t, J=3.2 Hz, 4H), 1.60-1.53 (m, 1H), 1.51-1.46 (m, 1H), 1.39-1.22 (m, 22H), 0.92-0.86 (m, 9H).

### Example 103—Compound 107

### O6-[2-[(2,2-difluorospiro[2.5]octane-6-carbonyl)oxymethyl]-2-[[6-[(Z)-non-3-enoxy]-6-oxo-hexanoyl]oxymethyl]-3-[4-(2-pyrrolidin-1-ylethyl carbamoyloxy)decanoyloxy]propyl]O1-[(Z)-non-3-enyl]hexanedioate

### Synthetic Scheme:

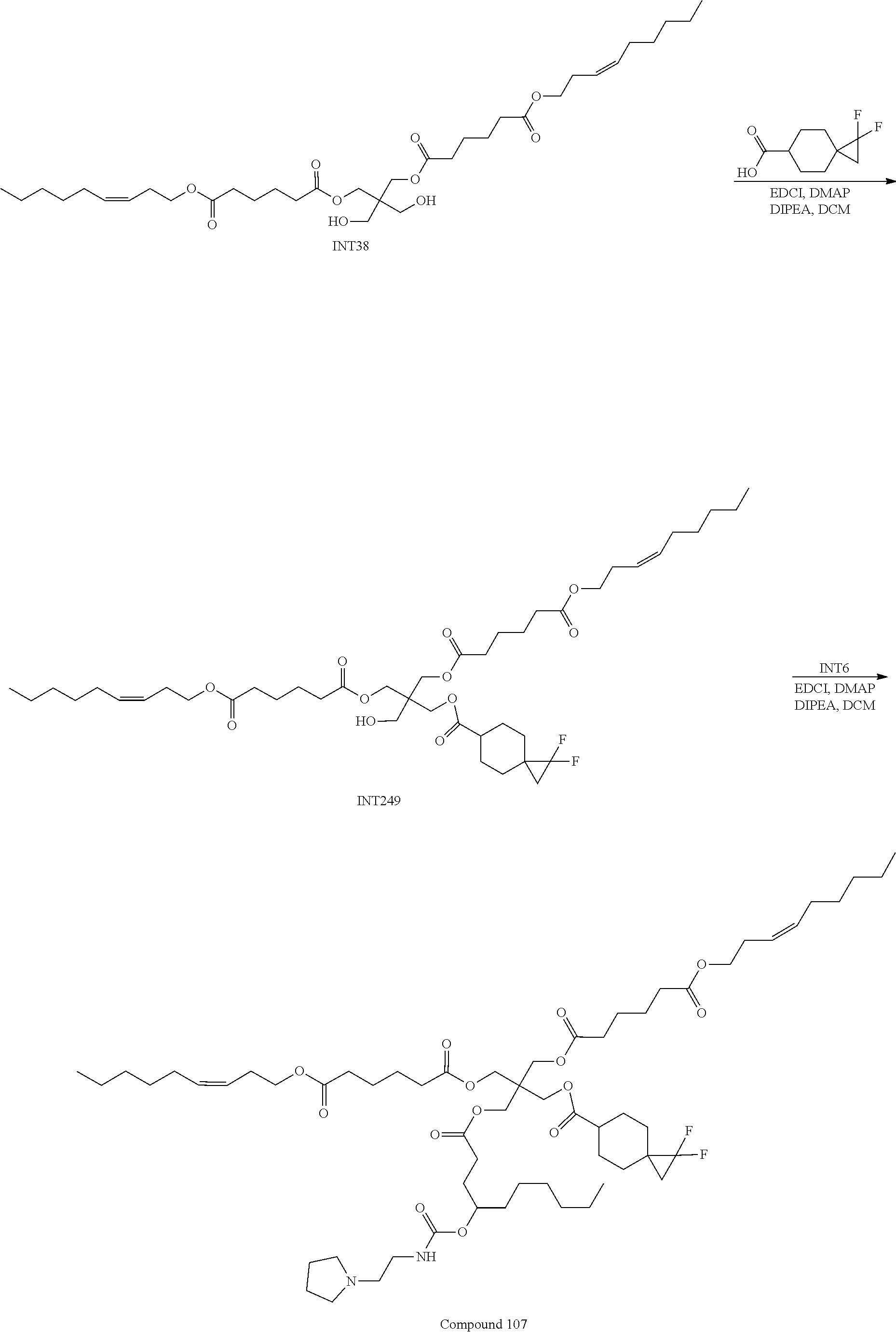

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (18 mL) was added EDCI (538.45 mg, 2.81 mmol, 1.2 eq), DMAP (28.60 mg, 234.07 mol, 0.1 eq), 2,2-difluorospiro[2.5]octane-6-carboxylic acid (356.13 mg, 1.87 mmol, 0.8 eq) and DIPEA (756.28 mg, 5.85 mmol, 1.02 mL, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 1/1). Compound O6-[2-[(2,2-difluorospiro[2.5]octane-6-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.5 g, 615.00 mol, 26.27% yield) was obtained as a colorless oil. ¹H NMR (400 MHz, CDCl₃) δ ppm 5.63-5.22 (m, 4H), 4.14-4.05 (m, 10H), 3.52 (s, 2H), 2.62 (s, 1H), 2.44 (s, 1H), 2.41-2.31 (m, 12H), 2.08-1.95 (m, 6H), 1.68-1.58 (m, 12H), 1.52-1.46 (m, 2H), 1.40-1.26 (m, 12H), 1.02 (t, J=8.2 Hz, 2H), 0.90 (t, J=6.8 Hz, 6H).

### Step 2:

To a solution of O6-[2-[(2,2-difluorospiro[2.5]octane-6-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.5 g, 615.00 mol, 1 eq) in DCM (11 mL) was added EDCI (141.47 mg, 737.99 mol, 1.2 eq), DMAP (7.51 mg, 61.50 mol, 0.1 eq), 4-(2-pyrrolidin-1-ylethylcarbamoyloxy)decanoic acid (242.39 mg, 737.99 mol, 1.2 eq) and DIPEA (198.71 mg, 1.54 mmol, 267.80 L, 2.5 eq) under N₂ atmosphere. The reaction mixture was stirred at 25° C. for 10 hr under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(2,2-difluorospiro[2.5]octane-6-carbonyl)oxymethyl]-2-[[6-[(Z)-non-3-enoxy]-6-oxo-hexanoyl]oxymethyl]-3-[4-(2-pyrrolidin-1-ylethyl carbamoyloxy)decanoyloxy]propyl]O1-[(Z)-non-3-enyl]hexanedioate (0.15 g, 133.52 mol, 21.71% yield) was obtained as a colorless oil. ¹H NMR (400 MHz, CDCl₃) δ ppm 5.58-5.25 (m, 4H), 4.75 (s, 1H), 4.20-3.97 (m, 12H), 3.31 (s, 2H), 2.66- 2.30 (m, 20H), 2.07-2.01 (m, 4H), 1.98-1.75 (m, 8H), 1.70-1.45 (m, 18H), 1.40-1.26 (m, 20H), 1.02 (t, J=8.4 Hz, 2H), 0.93-0.85 (m, 9H).

### Example 104—Compound 108

### O6-[2-(bicyclo[3.2.0]heptane-3-carbonyloxymethyl)-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate

### Synthetic Scheme:

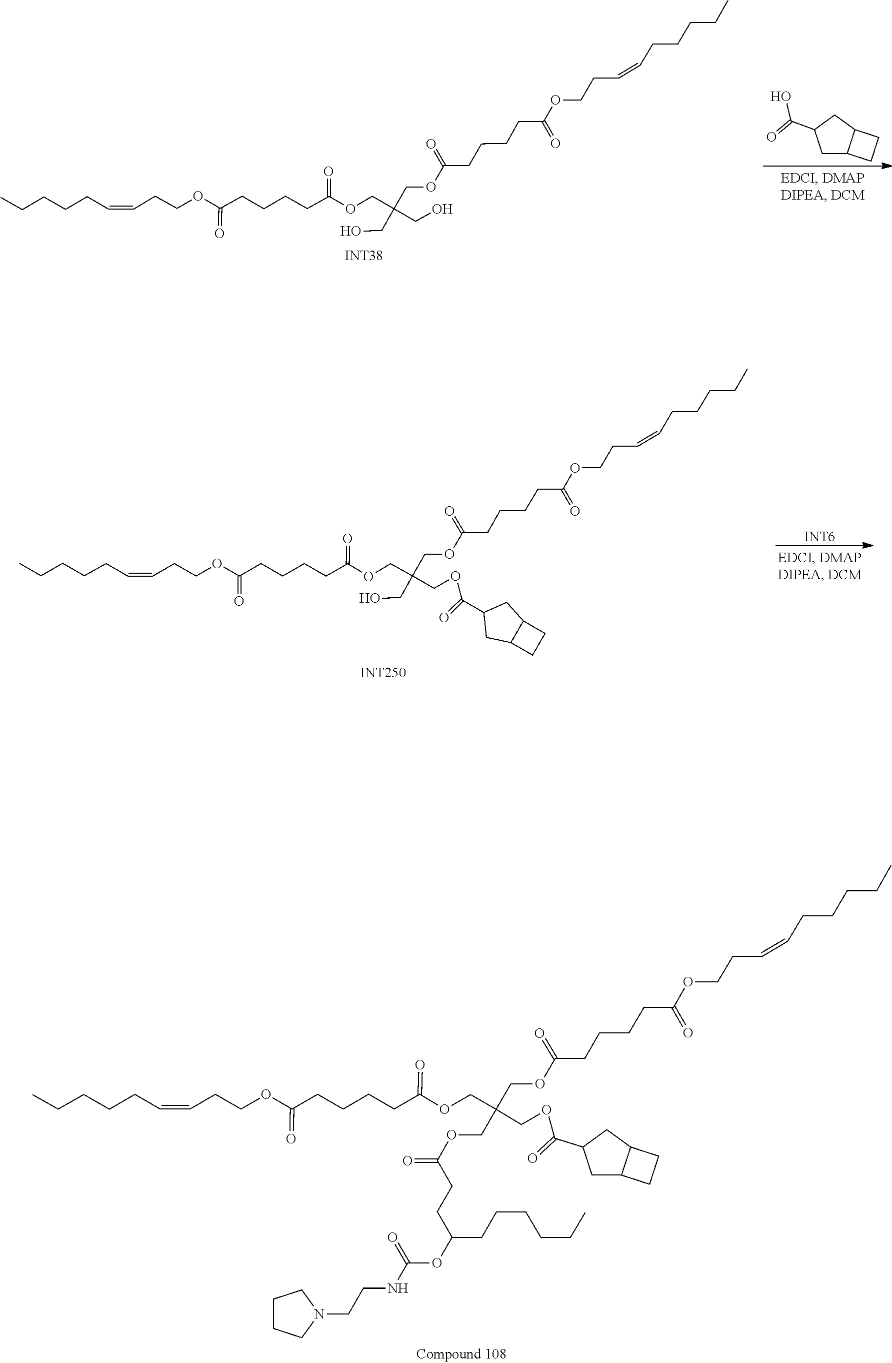

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), DMAP (28.60 mg, 234.07 mol, 0.1 eq) and bicyclo[3.2.0]heptane-3-carboxylic acid (328.11 mg, 2.34 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicycle[3.2.0]heptane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.31 mmol, 55.99% yield) was obtained as a colorless oil.

### Step 2:

To a solution of O6-[2-(bicyclo[3.2.0]heptane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.31 mmol, 1 eq) in DCM (10 mL) was added EDCI (301.49 mg, 1.57 mmol, 1.2 eq), DIPEA (423.46 mg, 3.28 mmol, 570.71 L, 2.5 eq), 4-pyrrolidin-1-ylpyridine (19.42 mg, 131.06 mol, 0.1 eq) and 4-(2-pyrrolidin-1-ylethylcarbamoyl-oxy)decanoic acid (430.46 mg, 1.31 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[3.2.0]heptane-3-carbonyloxymethyl)-2-[[6-[(Z)-non-3-enoxy]-6-oxo-hexanoyl]oxymethyl]-3-[4-(2-pyrrolidin-1ylethylcarbamoyloxy)decanoyloxy]propyl]O1-[(Z)-non-3-enyl]hexanedioate (0.16 g, 149.05 mol, 11.37% yield) was obtained as a colorless oil. ¹H NMR (400 MHz, CD₃OD) δ ppm 5.57-5.45 (m, 2H), 5.42-5.30 (m, 2H), 4.78-4.69 (m, 1H), 4.18 (d, J=4.2 Hz, 8H), 4.06 (t, J=6.8 Hz, 4H), 3.26 (t, J=6.8 Hz, 2H), 2.97-2.85 (m, 1H), 2.79-2.71 (m, 1H), 2.64-2.56 (m, 6H), 2.42-2.31 (m, 14H), 2.25-2.14 (m, 2H), 2.11-1.86 (m, 8H), 1.84-1.74 (m, 6H), 1.68-1.57 (m, 10H), 1.56-1.50 (m, 2H), 1.42-1.28 (m, 20H), 0.97-0.85 (m, 9H).

### Example 105—Compound 109

### O6-[2-(bicycle[3.2.1]octane-3-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

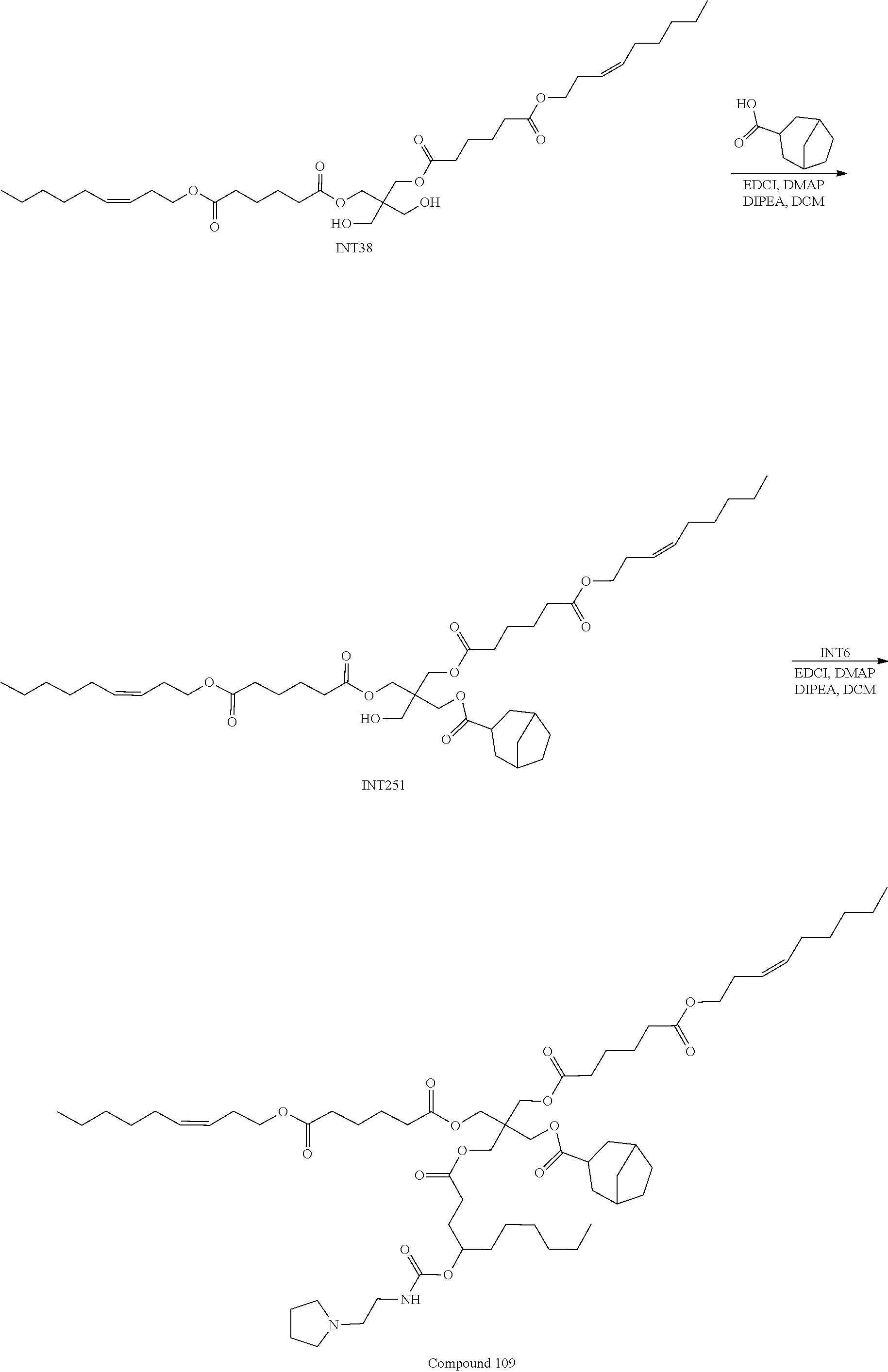

### Step 1:

To a solution of O6-[2,2-bis(hydroxymethyl)-3-[6-[(Z)-non-3-enoxy]-6-oxo-hexanoyl]oxy-propyl]O1-[(Z)-non-3-enyl]hexanedioate (1.5 g, 2.34 mmol, 1 eq) in DCM (15 mL) was added EDCI (538.45 mg, 2.81 mmol, 1.2 eq), DIPEA (756.28 mg, 5.85 mmol, 1.02 mL, 2.5 eq), DMAP (28.60 mg, 234.07 mol, 0.1 eq) and bicyclo[3.2.1]octane-3-carboxylic acid (360.94 mg, 2.34 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(bicyclo[3.2.1]-octane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.29 mmol, 54.98% yield) was obtained as a colorless oil. ¹H NMR (400 MHz, CDCl₃) δ ppm 5.58-5.46 (m, 2H), 5.40-5.28 (m, 2H), 4.20-4.00 (m, 10H), 3.57-3.49 (m, 2H), 2.68-2.52 (m, 2H), 2.44-2.29 (m, 12H), 2.26-2.14 (m, 4H), 2.09-1.99 (m, 4H), 1.78-1.71 (m, 2H), 1.70-1.63 (m, 8H), 1.60-1.53 (m, 2H), 1.48-1.23 (m, 16H), 0.90 (t, J=6.8 Hz, 6H).

### Step 2:

To a solution of O6-[2-(bicyclo[3.2.1]octane-3-carbonyloxymethyl)-2-(hydroxymethyl)-3-[6-[(Z)-non-3-enoxy]-6-oxo-hexanoyl]oxy-propyl]O1-[(Z)-non-3-enyl]hexanedioate (1 g, 1.29 mmol, 1 eq) in DCM (10 mL) was added EDCI (296.05 mg, 1.54 mmol, 1.2 eq), DIPEA (415.82 mg, 3.22 mmol, 560.40 L, 2.5 eq), DMAP (15.72 mg, 128.69 mol, 0.1 eq) and 4-(2-pyrrolidin-1-ylethylcarbamoyloxy)decanoic acid (422.69 mg, 1.29 mmol, 1 eq) under N₂ atmosphere. The reaction mixture was stirred at 25° C. for 10 hr under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 30%-70% B over 14.0 min) to get compound O6-[2-(bicycle[3.2.1]octane-3-carbonyloxymethyl)-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.16 g, 147.13 mol, 11.43% yield, HCl salt) as a colorless oil. ¹H NMR (400 MHz, CDCl₃) δ ppm 11.95 (s, 1H), 6.37 (s, 1H), 5.50-5.43 (m, 2H), 5.34-5.26 (m, 2H), 4.70 (s, 1H), 4.12-4.00 (m, 12H), 3.94-3.70 (m, 2H), 3.66-3.59 (m, 2H), 3.25 (d, J=4.4 Hz, 2H), 3.01-2.74 (m, 2H), 2.51 (t, J=7.8 Hz, 1H), 2.36-2.27 (m, 14H), 2.19-2.07 (m, 8H), 2.03-1.97 (m, 4H), 1.88-1.83 (m, 1H), 1.79-1.66 (m, 4H), 1.61 (s, 8H), 1.53-1.50 (m, 2H), 1.43 (d, J=8.8 Hz, 2H), 1.36-1.22 (m, 24H), 0.88-0.82 (m, 9H).

### Example 106—Compound 110

### O6-[2-[(7,7-dimethylnorbornane-1-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

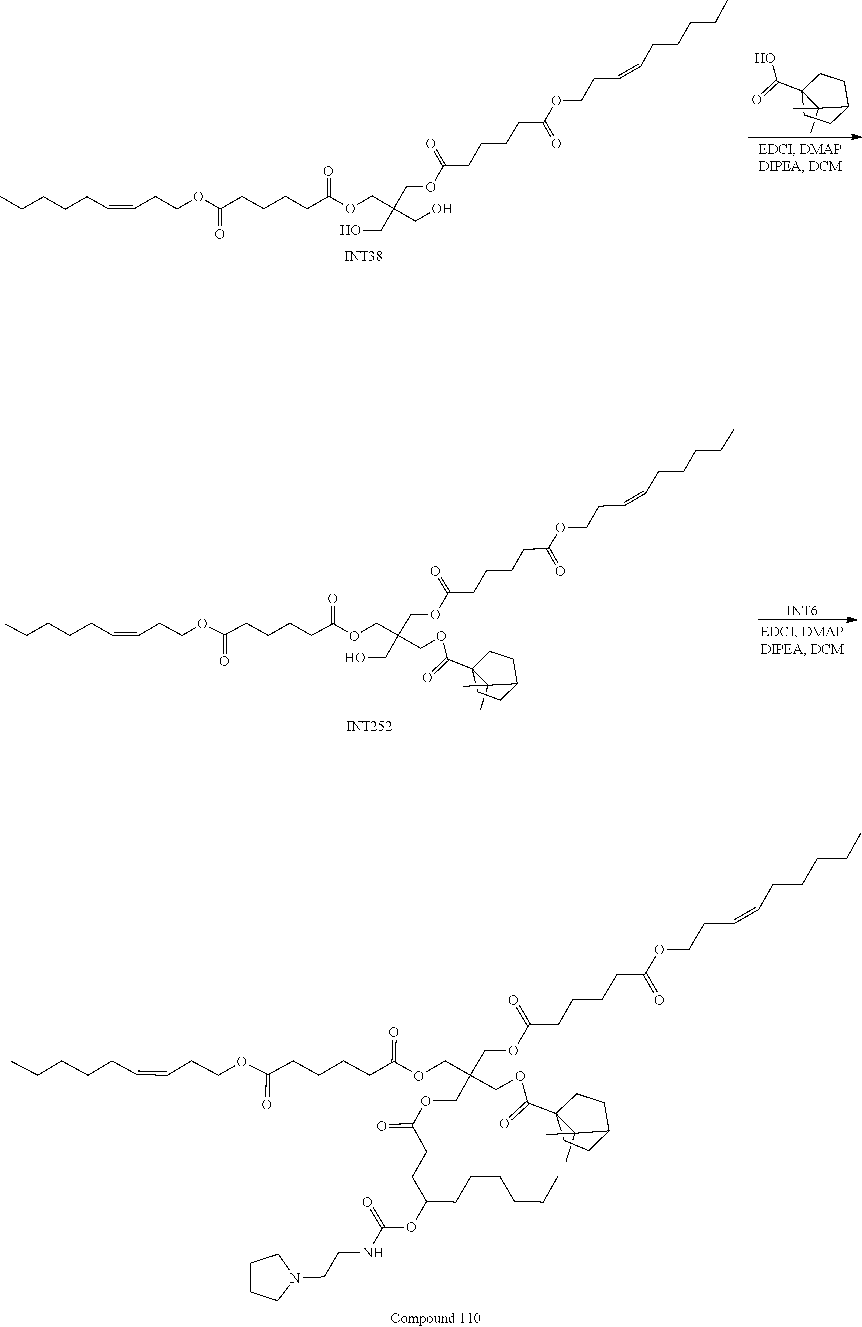

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 7,7-dimethylnorbornane-1-carboxylic acid (420.03 mg, 2.50 mmol, 0.8 eq), DMAP (38.13 mg, 312.09 mol, 0.1 eq), EDCI (717.93 mg, 3.75 mmol, 1.2 eq) and DIPEA (1.01 g, 7.80 mmol, 1.36 mL, 2.5 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[(7,7-dimethylnorbornane-1-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.07 mmol, 34.43% yield) was obtained as a colorless oil. ¹H NMR (400 MHz, CDCl₃) δ ppm 5.55-5.47 (m, 2H), 5.40-5.29 (m, 2H), 4.13-4.04 (m, 10H), 3.51 (s, 2H), 2.40-2.30 (m, 12H), 2.23-2.15 (m, 2H), 2.08-2.00 (m, 4H), 1.92-1.84 (m, 2H), 1.73 (t, J=4.2 Hz, 1H), 1.69-1.63 (m, 8H), 1.59-1.51 (m, 3H), 1.39-1.25 (m, 14H), 1.04 (s, 6H), 0.90 (t, J=6.8 Hz, 6H).

### Step 2:

A mixture of O6-[2-[(7,7-dimethylnorbornane-1-carbonyl)oxymethyl]-2-(hydroxymethyl)-3-[6-[(Z)-non-3-enoxy]-6-oxo-hexanoyl]oxy-propyl]O1-[(Z)-non-3-enyl]hexanedioate (0.85 g, 1.07 mmol, 1 eq), 4-(2-pyrrolidin-1-ylethylcarbamoyloxy)decanoic acid (423.50 mg, 1.29 mmol, 1.2 eq), DMAP (13.13 mg, 107.45 mol, 0.1 eq), EDCI (247.18 mg, 1.29 mmol, 1.2 eq) and DIPEA (347.18 mg, 2.69 mmol, 467.90 L, 2.5 eq) in DCM (8.5 mL) was stirred at 25° C. for 12 hours under N₂ atmosphere. The reaction mixture was diluted with H₂O (50 mL) and extracted with DCM 100 mL (50 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1) and prep-HPLC (column: X-Select CSH Phenyl-Hexyl 100*30 5u; mobile phase: [H₂O (0.04% HCl)-ACN:THF=1:1]; gradient: 50%-80% B over 12.0 min) to get compound O6-[2-[(7,7-dimethylnorbornane-1-carbonyl)oxymethyl]-2-[[6-[(Z)-non-3-enoxy]-6-oxo-hexanoyl]oxymethyl]-3-[4-(2-pyrrolidin-1-ylethylcarbamoyloxy)decanoyloxy]propyl]O1-[(Z)-non-3-enyl]hexanedioate (0.15 g, 136.18 mol, 12.67% yield, 100% purity, HCl salt) as a colorless oil. ¹H NMR (400 MHz, CDCl₃) δ ppm 12.59-12.22 (m, 1H), 6.29 (br s, 1H), 5.55-5.47 (m, 2H), 5.38-5.30 (m, 2H), 4.79-4.70 (m, 1H), 4.17-4.05 (m, 12H), 3.86 (br s, 2H), 3.67 (d, J=4.4 Hz, 2H), 3.31-3.21 (m, 2H), 2.91-2.80 (m, 2H), 2.39-2.30 (m, 14H), 2.25-2.01 (m, 10H), 1.96-1.71 (m, 8H), 1.68-1.63 (m, 8H), 1.58-1.47 (m, 4H), 1.33-1.25 (m, 18H), 1.02 (s, 6H), 0.92-0.86 (m, 9H).

### Example 107—Compound 111

### O6-[2-[[6-[(Z)-non-3-enoxy]-6-oxo-hexanoyl]oxymethyl]-2-[[4-(4pentylcyclohexyl)cyclohexanecarbonyl]oxymethyl]-3-[4-(2-pyrrolidin-1-ylethylcarbamoyloxy)decanoyloxy]propyl]O1-[(Z)-non-3-enyl]hexanedioate

### Synthetic Scheme:

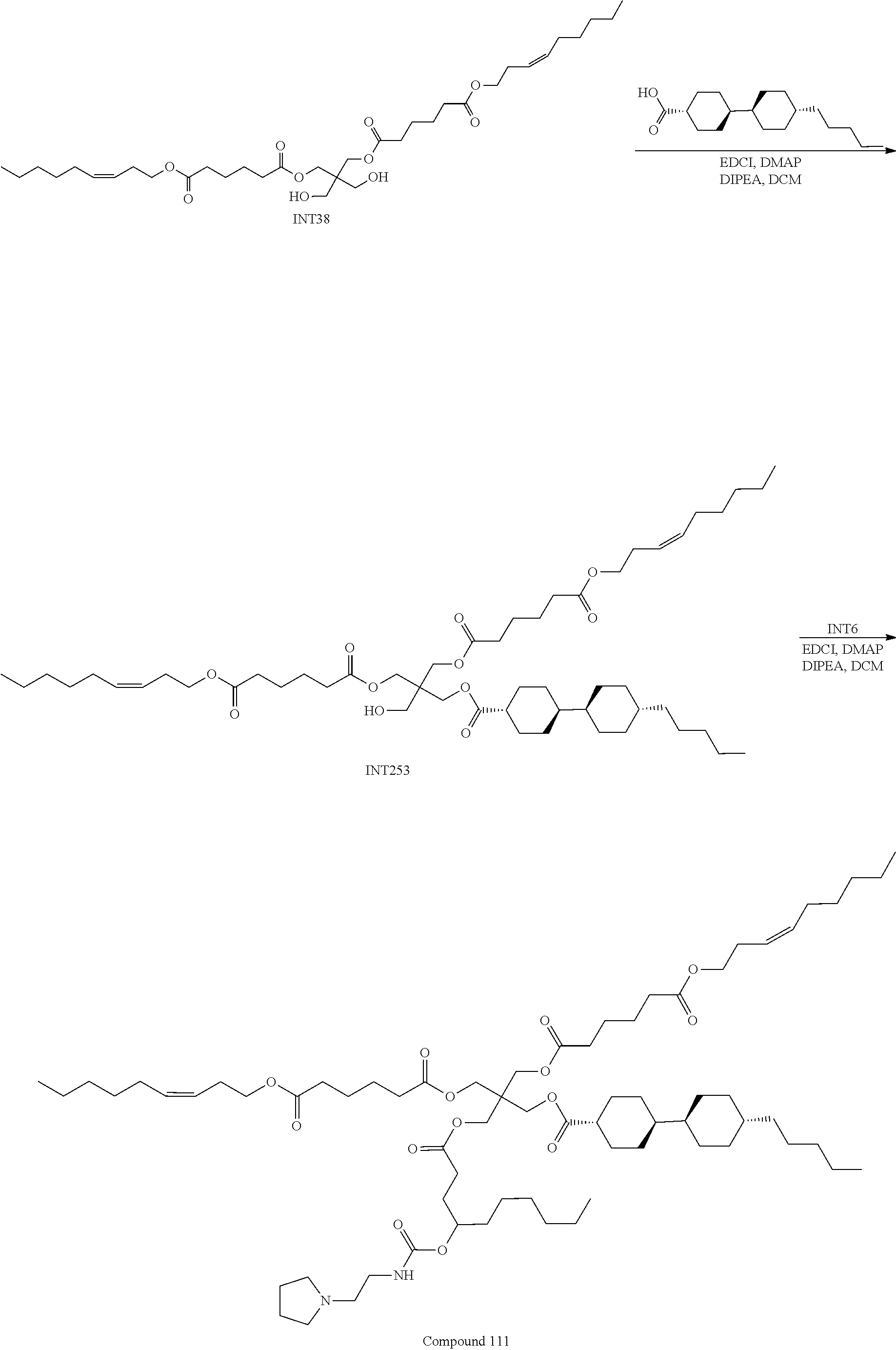

### Step 1:

A mixture of O6-[2,2-bis(hydroxymethyl)-3-[6-[(Z)-non-3-enoxy]-6-oxo-hexanoyl]oxy-propyl]O1-[(Z)-non-3-enyl]hexanedioate (2 g, 3.12 mmol, 1 eq), 4-(4-pentylcyclohexyl)cyclohexanecarboxylic acid (525.14 mg, 1.87 mmol, 0.6 eq), EDCI (717.93 mg, 3.75 mmol, 1.2 eq), DIPEA (1.01 g, 7.80 mmol, 1.36 mL, 2.5 eq) and DMAP (38.13 mg, 312.09 mol, 0.1 eq) in DCM (20 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-(hydroxymethyl)-2-[[6-[(Z)-non-3-enoxy]-6-oxo-hexanoyl]oxymethyl]-3-[4-(4-pentylcyclohexyl)cyclohexanecarbonyl]oxy-propyl]O1-[(Z)-non-3-enyl]hexanedioate (0.85 g, 941.02 mol, 30.15% yield) was obtained as a colorless oil. ¹H NMR (400 MHz, CDCl₃) δ ppm 5.57-5.47 (m, 2H), 5.39-5.31 (m, 2H), 4.12-4.05 (m, 10H), 3.50 (s, 2H), 2.39-2.31 (m, 12H), 2.07-1.96 (m, 6H), 1.82-1.70 (m, 6H), 1.68-1.64 (m, 10H), 1.39-1.22 (m, 24H), 1.17-1.14 (m, 2H), 1.03-0.95 (m, 4H), 0.91-0.87 (m, 9H).

### Step 2:

A mixture of O6-[2-(hydroxymethyl)-2-[[6-[(Z)-non-3-enoxy]-6-oxo-hexanoyl]oxymethyl]-3-[4-(4-pentylcyclohexyl)cyclohexanecarbonyl]oxy-propyl]O1-[(Z)-non-3-enyl]hexanedioate (0.85 g, 941.02 mol, 1 eq), 4-(2-pyrrolidin-1-ylethylcarbamoyloxy)decanoic acid (370.89 mg, 1.13 mmol, 1.2 eq), EDCI (216.47 mg, 1.13 mmol, 1.2 eq), DMAP (11.50 mg, 94.10 mol, 0.1 eq) and DIPEA (304.05 mg, 2.35 mmol, 409.77 L, 2.5 eq) in DCM (8.5 mL) was stirred at 25° C. for 12 h under N₂ atmosphere. The reaction mixture was diluted with H₂O (100 mL) and extracted with DCM 200 mL (100 mL*2). The combined organic layers were dried over Na₂SO₄, filtered and the filtrate was concentrated under reduced pressure to give a residue. The residue was purified by column chromatography (SiO₂, Petroleum ether/Ethyl acetate=100/1 to 0/1). Compound O6-[2-[[6-[(Z)-non-3-enoxy]-6-oxo-hexanoyl]oxymethyl]-2-[[4-(4pentylcyclohexyl)cyclohexanecarbonyl]oxymethyl]-3-[4-(2-pyrrolidin-1-ylethylcarbamoyloxy)decanoyloxy]propyl]O1-[(Z)-non-3-enyl]hexanedioate (0.15 g, 123.59 mol, 13.13% yield, 100% purity) was obtained as a colorless oil. ¹H NMR (400 MHz, CDCl₃) δ ppm 5.56-5.47 (m, 2H), 5.38-5.31 (m, 2H), 5.27-5.10 (m, 1H), 4.80-4.70 (m, 1H), 4.13-4.05 (m, 12H), 3.37-3.22 (m, 2H), 2.61-2.50 (m, 4H), 2.40-2.31 (m, 14H), 2.25-2.16 (m, 2H), 2.07-2.01 (m, 4H), 1.98 (s, 1H), 1.95 (d, J=1.6 Hz, 1H), 1.81-1.74 (m, 8H), 1.65 (s, 12H), 1.57-1.47 (m, 4H), 1.36-1.25 (m, 28H), 1.15 (s, 4H), 1.04 (s, 2H), 1.00 (s, 2H), 0.98 (d, J=3.2 Hz, 2H), 0.91-0.87 (m, 12H).

We claim:

1. A compound of the following formula: 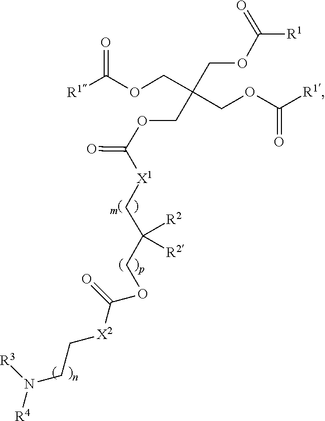
or a pharmaceutically acceptable salt thereof; wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl, optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH—(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl, optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹;
R² is C₁-C₁₂ alkyl, C₂-C₁₂ alkenyl, C₂-C₁₂ alkynyl, C₁-C₁₂ alkoxy, (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R^{2′} is hydrogen, C₁-C₁₂ alkyl, alkenyl, or alkynyl, C₁-C₁₂ alkoxy, (C₁-C₄ alkyl)-(C₁-C₄ alkoxy);
wherein R² and R^{2′} can combine to form an optionally substituted C₄-C₆ cycloalkyl, an optionally substituted C₃-C₆ cycloalkyl, or an optionally substituted C₃-C₆ heterocycle;
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹¹ is hydrogen or C₁-C₆ alkyl, or wherein R¹¹ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl, or an optionally substituted C₅-C₆ aryl;
R¹⁴ is hydrogen or C₁-C₆ alkyl, or wherein R¹⁴ and R² join together to form an optionally substituted C₅-C₈ cycloalkyl;
m is 1-4;
p is 0-4; and
n is 1-5.

2. A compound of the following formula: 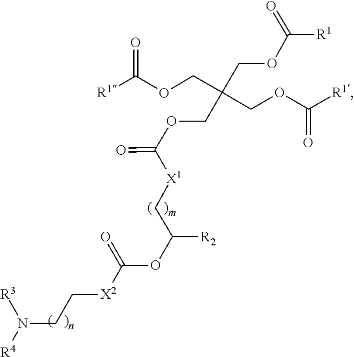
or a pharmaceutically acceptable salt thereof, wherein:
each of R¹ and R^{1′} is independently: (C₁-C₉ alkyl)-R⁵, (C₂-C₉ alkenyl)-R⁵, (C₂-C₉ alkynyl)-R⁵, or (C₁-C₈ alkoxy)-R⁵;
R^{1″} is independently: (C₁-C₉ alkyl)-R⁵, (C₂-C₉ alkenyl)-R⁵, (C₂-C₉ alkynyl)-R⁵, (C₁-C₈ alkoxy)-R⁵, or R¹²—R³;
each R⁵ is independently: hydrogen, C₁-C₁₂ alkyl, C₂-C₁₂ alkenyl, C₂-C₁₂ alkynyl, C₂-C₁₂ alkoxy, optionally substituted C₃-C₁₂ cycloalkyl, optionally substituted C₅-C₆ aryl, 1-adamantyl, 2-adamantyl, sterolyl, C(O)O—R⁶, OC(O)—R⁶, OC(O)O—R⁶, CH(R⁷)R⁸, C(O)O—CH(R⁷)R⁸, C(O)O—C₁-C₄ alkyl-(R⁹)R¹⁰, OC(O)—C₁-C₄ alkyl-(R⁹)R¹⁰, or OC(O)CH(R⁹)R¹⁰;
each R⁶ is independently: C₇-C₁₂ alkyl, C₇-C₁₂ alkenyl, C₇-C₁₂ alkynyl, C₇-C₁₂ alkoxy, optionally substituted C₃-C₁₂ cycloalkyl, optionally substituted C₅-C₆ aryl, 1-adamantyl, 2-adamantyl, or sterolyl;
each R⁷ and R⁸ is independently: C₇-C₁₂ alkyl, C₇-C₁₂ alkenyl, C₇-C₁₂ alkynyl, or C₇-C₁₂ alkoxy;
each R⁹ and R¹⁰ is independently: C₁-C₁₂ alkyl or C₂-C₁₂ alkenyl;
X¹ is O, NH, or CHR¹⁴;
X² is O, NH, or CHR¹¹;
R² is C₁-C₁₂ alkyl, C₂-C₁₂ alkenyl, or C₂-C₁₂ alkynyl, C₁-C₁₂ alkoxy, (C₁-C₄ alkyl)-(C₁-C₄ alkoxy), optionally substituted C₃-C₁₂ cycloalkyl, (C₁-C₄ alkyl)-(optionally substituted C₃-C₁₂ cycloalkyl), optionally substituted C₃-C₆ heterocycle, (C₁-C₄ alkyl)-(optionally substituted C₃-C₆ heterocycle), optionally substituted C₅-C₆ aryl, or (C₁-C₄ alkyl)-(optionally substituted C₅-C₆ aryl);
R³ and R⁴ are each independently: hydrogen, C₁-C₆ alkyl, C₁-C₆ alkoxy, or C₁-C₆ hydroxyalkyl, or wherein R³ and R⁴ join together to form a heterocyclic ring comprising one or more N, O, or S heteroatoms;
R¹² is a bond or an optionally substituted, branched or unbranched C₁-C₆ alkyl, C₁-C₆ alkenyl, or C₁-C₆ alkynyl;
R¹³ is hydrogen, an optionally substituted C₃-C₁₂ cycloalkyl, or an optionally substituted C₅-C₆ aryl;
R¹¹ is hydrogen or C₁-C₆ alkyl, or wherein R¹¹ and R⁴ join together to form a heterocyclic ring comprising one or more N, O, or S heteroatoms;
R¹⁴ is hydrogen or C₁-C₆ alkyl, or wherein R¹⁴ and R² join together to form an optionally substituted C₅-C₈ cycloalkyl;
n is 1-5; and
m is 1-4.

3. The compound of claim 1, wherein two of R¹, R^{1′}, and R^{1″} are each independently (C₁-C₉ alkyl)-R⁵, and each R⁵ is independently C₂-C₁₂ alkenyl.

4. The compound of claim 3, wherein each of R¹, R^{1′}, and R^{1″} is independently (C₁-C₉ alkyl)-R⁵; and each R⁵ is independently C₂-C₁₂ alkenyl.

5. The compound of claim 3, wherein one of R¹, R^{1′}, and R^{1″} is (C₁-C₉ alkyl)-R^{5′}; and R^{5′} is C₆-C₁₀ cycloalkyl; optionally wherein the R^{5′} is 1-adamantyl or 2-adamantyl.

6. The compound of claim 3, wherein one of R¹, R^{1′}, and R^{1″} is CH(R⁷)R⁸; and each R⁷ and R⁸ is independently C₇-C₁₂ alkoxy.

7. The compound of claim 1, wherein two of R¹, R^{1′}, and R^{1″} are independently (C₁-C₉ alkyl)-R⁵; each R⁵ is independently C(O)O—R⁶; and each R⁶ is independently C₇-C₁₂ alkyl or C₇-C₁₂ alkenyl.

8. The compound of claim 7, wherein each of R¹, R^{1′}, and R^{1″} is independently (C₁-C₉ alkyl)-R⁵; each R⁵ is independently C(O)O—R⁶; and each R⁶ is independently C₇-C₁₂ alkyl or C₇-C₁₂ alkenyl.

9. The compound of claim 6, wherein one of R¹, R^{1′}, and R^{1″} is (C₁-C₉ alkyl)-R^{5′}; and R^{5′} is C₆-C₁₀ cycloalkyl; optionally wherein the R^{5′} is 1-adamantyl or 2-adamantyl.

10. The compound of claim 7, wherein one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy.

11. The compound of claim 7, wherein one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

12. The compound of claim 7, wherein R¹ and R^{1′} are (C₁-C₉ alkyl)-R^{5′}, R^{5′} is C(O)O—R⁶, and each R⁶ is independently C₇-C₁₂ alkyl or C₇-C₁₂ alkenyl; and R^{1′} is R¹²—R¹³, wherein R¹² is a bond or an optionally substituted, branched or unbranched C₁-C₆ alkyl, C₁-C₆ alkenyl, or C₁-C₆ alkynyl; and R¹³ is hydrogen, an optionally substituted C₃-C₁₂ cycloalkyl, or an optionally substituted C₅-C₆ aryl.

13. The compound of claim 12, wherein R¹² is a bond.

14. The compound of claim 12, wherein R¹² is C₁-C₆ alkyl; optionally C₁ alkyl.

15. The compound of claim 12, wherein R¹² is an optionally substituted, branched or unbranched C₁-C₆ alkyl, C₁-C₆ alkenyl, or C₁-C₆ alkynyl; optionally wherein R¹² is selected from pentyl, 1-methylpentyl, 4-methylpentyl, 5,5,5-trifluoropentyl, 4,4,5,5,5-pentafluoropentyl, and pent-4-ynyl.

16. The compound of any one of claims 12 to 14, wherein R¹³ is hydrogen.

17. The compound of any one of claims 13 to 14, wherein R¹³ is an optionally substituted C₃-C₁₂ cycloalkyl; optionally wherein R¹³ is selected from optionally substituted cyclopropane, optionally substituted cyclobutane, and optionally substituted cyclohexane such as 4-pentylcyclohexyl.

18. The compound of 17, wherein R¹³ is an optionally substituted fused C₃-C₁₂ cycloalkyl, an optionally substituted bridged C₃-C₁₂ cycloalkyl, or an optionally substituted spiro C₃-C₁₂ cycloalkyl.

19. The compound of any one of claims 13 to 14, wherein R¹³ is an optionally substituted C₅-C₆ aryl; optionally an optionally substituted phenyl such as 4-pentylphenyl or 3,5-di-tert-butylphenyl.

20. The compound of any one of claims 13 to 14, wherein R¹³ is an optionally substituted bicyclo[2.2.2]pentane; optionally wherein R¹³ is an unsubstituted bicyclo[2.2.2]pentane, 1-(trifluoromethyl)bicyclo[1.1.1]pentane, or 1-methylbicyclo[1.1.1]pentane.

21. The compound of any one of claims 13 to 14, wherein R¹³ is an optionally substituted bicyclo[2.1.0]pentane; optionally wherein R¹³ is an unsubstituted bicyclo[2.1.0]pentane.

22. The compound of any one of claims 13 to 14, wherein R¹³ is an optionally substituted bicyclo[3.1.0]hexane; optionally wherein R¹³ is 6,6-difluorobicyclo[3.1.0]hexane.

23. The compound of any one of claims 13 to 14, wherein R¹³ is an optionally substituted bicyclo[2.1.1]hexane; optionally wherein R¹³ is unsubstituted bicyclo[2.1.1]hexane or 1-fluorobicyclo[2.1.1]hexane.

24. The compound of any one of claims 13 to 14, wherein R¹³ is an optionally substituted spiro[2.3]hexane; optionally unsubstituted spiro[2.3]hexane or 1,1-difluorospiro[2.3]hexane.

25. The compound of any one of claims 13 to 14, wherein R¹³ is an optionally substituted 1,1′-bi(cyclohexane).

26. The compound of any one of claims 13 to 14, wherein R¹³ is an optionally substituted decahydronaphthalene.

27. The compound of any one of claims 13 to 14, wherein R¹³ is an optionally substituted bicyclo[2.2.1]heptane; optionally wherein R¹³ is an unsubstituted bicyclo[2.2.1]heptane or 7,7-dimethylbicyclo[2.2.1]heptane.

28. The compound of any one of claims 13 to 14, wherein R¹³ is an optionally substituted bicyclo[4.1.0]heptane; optionally wherein R¹³ is unsubstituted bicyclo[4.1.0]heptane or 7,7-difluorobicyclo[4.1.0]heptane.

29. The compound of any one of claims 13 to 14, wherein R¹³ is an optionally substituted bicyclo[3.2.0]heptane; optionally wherein R¹³ is unsubstituted bicyclo[3.2.0]heptane.

30. The compound of any one of claims 13 to 14, wherein R¹³ is an optionally substituted spiro[3.3]heptane; optionally wherein R¹³ is unsubstituted spiro[3.3]heptane or 2,2-difluorospiro[3.3]heptane.

31. The compound of any one of claims 13 to 14, wherein R¹³ is an optionally substituted bicyclo[2.2.2]octane; optionally wherein R¹³ is an unsubstituted bicyclo[2.2.2]octane or 1-methylbicyclo[2.2.2]octane.

32. The compound of any one of claims 13 to 14, wherein R¹³ is an optionally substituted bicyclo[3.2.1]octane; optionally wherein R¹³ is unsubstituted bicyclo[3.2.1]octane or 8-oxabicyclo[3.2.1]octane.

33. The compound of any one of claims 13 to 14, wherein R¹³ is an optionally substituted spiro[2.5]octane; optionally wherein R¹³ is unsubstituted spiro[2.5]octane or 1,1-difluorospiro[2.5]octane.

34. The compound of any one of claims 13 to 14, wherein R¹³ is an optionally substituted bicyclo[3.2.2]nonane; optionally wherein R¹³ is unsubstituted bicyclo[3.2.2]nonane or 1-fluorobicyclo[3.2.2]nonane.

35. The compound of any one of claims 13 to 14, wherein R¹³ is an optionally substituted 1-bicyclo[3.3.1]nonane; optionally wherein R¹³ is unsubstituted bicyclo[3.3.1]nonane or 1-methylbicyclo[3.3.1]nonane.

36. The compound of any one of claims 13 to 14, wherein R¹³ is adamantane.

37. The compound of claim 1, wherein two of R¹, R^{1′}, and R^{1″} are independently (C₁-C₉ alkyl)-R⁵; each R⁵ is independently OC(O)CH(R⁹)R¹⁰; and each R⁹ and R¹⁰ is independently C₁-C₁₂ alkyl or C₂-C₁₂ alkenyl.

38. The compound of claim 37, wherein each of R¹, R^{1′}, and R^{1″} is independently (C₁-C₉ alkyl)-R⁵; R⁵ is OC(O)CH(R⁹)R¹⁰; and each R⁹ and R¹⁰ is independently C₁-C₁₂ alkyl or C₂-C₁₂ alkenyl.

39. The compound of claim 1, wherein one of R¹, R^{1′}, and R^{1″} is (C₁-C₉ alkyl)-R^{5′}; R^{5′} is C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl.

40. The compound of claim 1, wherein two of R¹, R^{1′}, and R^{1″} are independently (C₁-C₉ alkyl)-R⁵; each R⁵ is independently C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl.

41. The compound of claim 1, wherein each of R¹, R^{1′}, and R^{1″} is independently (C₁-C₉ alkyl)-R⁵; R⁵ is C(O)O—C₁-C₄ alkyl-(R⁹)R¹⁰ or OC(O)—C₁-C₄ alkyl-(R⁹)R¹⁰; and each R⁹ and R¹⁰ is independently C₁-C₁₂ alkyl or C₂-C₁₂ alkenyl.

42. The compound of claim 37, wherein one of R¹, R^{1′}, and R^{1″} is (C₁-C₉ alkyl)-R^{5′}; and R^{5′} is C₆-C₁₀ cycloalkyl; optionally wherein R^{5′} is 1-adamantyl or 2-adamantyl.

43. The compound of claim 37, wherein one of R¹, R^{1′}, and R^{1″} is (C₁-C₉ alkyl)-R^{5′}; R^{5′} is C(O)O—R⁶; and R⁶ is C₇-C₁₂ alkyl or C₇-C₁₂ alkenyl.

44. The compound of claim 37, wherein one of R¹, R^{1′}, and R^{1″} is (C₁-C₉ alkyl)-R^{5′}; R^{5′} is CH(R⁷)R⁸; and each R⁷ and R⁸ is independently C₇-C₁₂ alkoxy.

45. The compound of claim 1, wherein two of R¹, R^{1′}, and R^{1″} are independently (C₁-C₉ alkyl)-R⁵; each R⁵ is independently CH(R⁷)R⁸; and each R⁷ and R⁸ is independently C₇-C₁₂ alkoxy.

46. The compound of claim 45, wherein each of R¹, R^{1′}, and R^{1″} is independently (C₁-C₉ alkyl)-R⁵; each R⁵ is independently CH(R⁷)R⁸; and each R⁷ and R⁸ is independently C₇-C₁₂ alkoxy.

47. The compound of claim 42, wherein one of R¹, R^{1′}, and R^{1″} is (C₁-C₉ alkyl)-R^{5′}; R^{5′} is OC(O)CH(R⁹)R¹⁰; and each R⁹ and R¹⁰ is independently C₁-C₁₂ alkyl or C₂-C₁₂ alkenyl.

48. The compound of any one of claims 1-47, wherein R² is C₁-C₁₂ alkyl, C₂-C₁₂ alkenyl, or C₂-C₁₂ alkynyl.

49. The compound of any one of claims 1-47, wherein R² is C₄-C₈ alkyl.

50. The compound of any one of claims 1-47, wherein R² is methyl, ethyl, propyl, isopropyl, butyl, 1-isobutyl, 2-isobutyl, tert-butyl, C₅ alkyl, C₆ alkyl, C₈ alkyl, or C₁₀ alkyl.

51. The compound of any one of claims 1-47, wherein R² is C₁-C₁₂ alkoxy or (C₁-C₄ alkyl)-(C₁-C₄ alkoxy); optionally wherein R² is methoxy, ethoxy, methoxymethyl, or ethoxyethyl.

52. The compound of any one of claims 1-47, wherein R² is optionally substituted C₃-C₁₂ cycloalkyl, optionally substituted C₃-C₆ heterocycle, or optionally substituted C₅-C₆ aryl; optionally wherein R² is optionally substituted cyclopropyl, cyclobutyl, cyclopentyl, or cyclohexyl such as 4-pentylcyclohexyl.

53. The compound of any one of claims 1-47, wherein R² is optionally substituted phenyl; optionally wherein R² is 4-pentylphenyl.

54. The compound of any one of claims 1-47, wherein R² is optionally substituted (C₁-C₄ alkyl)-(optionally substituted C₃-C₁₂ cycloalkyl), (C₁-C₄ alkyl)-(optionally substituted C₃-C₆ heterocycle), or (C₁-C₄ alkyl)-(optionally substituted C₅-C₆ aryl); optionally wherein R² is —CH₂-cyclopropyl, —(CH₂)₂-cyclopropyl, —CH₂-cyclohexyl, —(CH₂)₂-cyclohexyl, —(CH₂)₂-(4-pentylcyclohexyl), —CH₂-phenyl, or —(CH₂)₂-phenyl.

55. The compound of any one of claims 48-54, wherein R^{2′} is hydrogen.

56. The compound of any one of claims 48-54, wherein R^{2′} is an C₁-C₁₂ alkyl, C₂-C₁₂ alkenyl, or C₂-C₁₂ alkynyl; optionally wherein R² is methoxy, ethoxy, methoxymethyl, or ethoxyethyl.

57. The compound of any one of claims 1-47, wherein R² and R^{2′} combine to form an optionally substituted C₄-C₆ cycloalkyl or C₄-C₆ heterocycle; optionally wherein R² and R^{2′} combine to form cyclohexane or pyran.

58. The compound of any one of claims 1-57, wherein X¹ is CH₂.

59. The compound of any one of claims 1-57, wherein X¹ is CHR¹⁴, and R¹⁴ and R² join together to form an optionally substituted C₅-C₈ cycloalkyl; optionally wherein R¹⁴ and R² join together to form an optionally substituted C₅ cycloalkyl or optionally substituted C₆ cycloalkyl.

60. The compound of any one of claims 1-59, wherein X² is NH.

61. The compound of any one of claims 1-59, wherein X² is O.

62. The compound of any one of claims 1-61, wherein R³ and R⁴ are each independently C₁-C₆ alkyl.

63. The compound of any one of claims 1-61, wherein R³ and R⁴ are joined together to form a heterocyclic ring comprising a nitrogen heteroatom; optionally wherein R³ and R⁴ are joined together to form pyrrolidine or wherein R³ and R⁴ are joined together with a preceding alkyl group to form quinuclidine.

64. The compound of any one of claims 1-63, wherein n is 1, 2, 3, or 4.

65. The compound of any one of claims 1-64, wherein m is 1, 2, 3 or 4.

66. The compound of any one of claims 1-65, wherein p is 0, 1, 2, 3 or 4.

67. The compound of any one of claims 1-66, wherein two of R¹, R^{1′}, and R^{1″} are independently (C₂ alkyl)-R⁵.

68. The compound of any one of claims 1-66, wherein two of R¹, R^{1′}, and R^{1″} are independently (C₃ alkyl)-R⁵.

69. The compound of any one of claims 1-66, wherein two of R¹, R^{1′}, and R^{1″} are independently (C₄ alkyl)-R⁵.

70. The compound of any one of claims 1-66, wherein two of R¹, R^{1′}, and R^{1″} are independently (C₅ alkyl)-R⁵.

71. The compound of any one of claims 1-66, wherein two of R¹, R^{1′}, and R^{1″} are independently (C₆ alkyl)-R⁵.

72. The compound of any one of claims 1-66, wherein two of R¹, R^{1′}, and R^{1″} are independently (C₇ alkyl)-R⁵.

73. The compound of any one of claims 1-66, wherein two of R¹, R^{1′}, and R^{1″} are independently (C₈ alkyl)-R⁵.

74. The compound of any one of claims 1-66, wherein the compound is of the following formula: 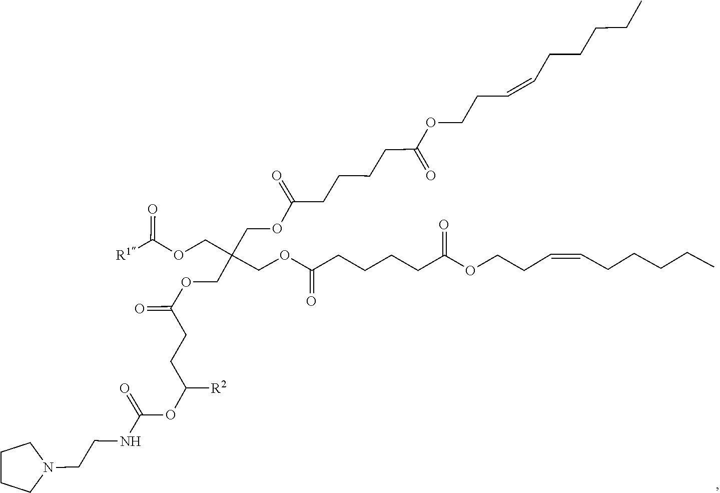
or a pharmaceutically acceptable salt thereof.

75. The compound of any one of claims 1-66, wherein the compound is of the following formula: 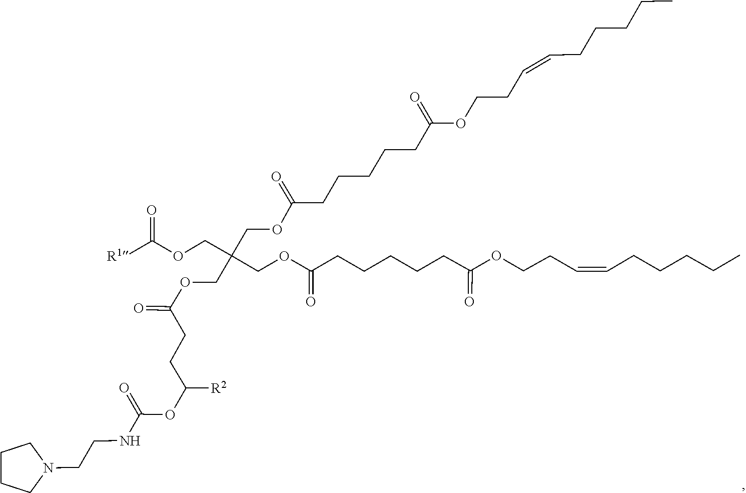
or a pharmaceutically acceptable salt thereof.

76. The compound of any one of claims 1-66, wherein the compound is of the following formula: 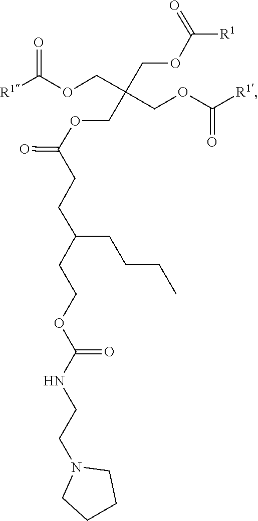
or a pharmaceutically acceptable salt thereof.

77. The compound of claim 1, wherein the compound is any one of Compounds 1-209 or a pharmaceutically acceptable salt thereof.

78. The compound of claim 1, wherein the compound is any one of Compounds 7, 8, 10, 13, 14, 26, 33, 38, 39, 40, 48, 60, 61, 89, 103, or 109, or a pharmaceutically acceptable salt thereof.

79. A lipid nanoparticle comprising the compound of any one of claims 1-78.

80. A lipid nanoparticle comprising the compound of any one of claims 1-78; a phospholipid;
a cholesterol; and a polyethylene glycol lipid.

81. A lipid nanoparticle comprising: about 20-80 mol % of the compound of any one of claims 1-78, about 7.5-40 mol % of phospholipid, about 6-45 mol % of cholesterol, and about 1-4 mol % of PEG lipid.

82. A lipid nanoparticle comprising: about 45-65 mol % of the compound of any one of claims 1-78, about 10 mol % of phospholipid, about 25-45 mol % of cholesterol, and about 1-4 mol % of PEG lipid.

83. A lipid nanoparticle comprising: from about 45-50 mol % of the compound of any one of claims 1-78, about 10 mol % of phospholipid, about 38-42 mol % of cholesterol, and from about 2-3 mol % of PEG lipid.

84. The lipid nanoparticle of claim 83, comprising: about 47.5 mol % of the compound of any one of claims 1-78, about 40 mol % of cholesterol, and about 2.5 mol % of PEG lipid.

85. A lipid nanoparticle comprising: about 47.5-52.5 mol % of the compound of any one of claims 1-78, about 10 mol % of phospholipid, about 37-40 mol % of cholesterol, and about 1-2 mol % of PEG lipid.

86. The lipid nanoparticle of claim 85, comprising: about 50 mol % of the compound of any one of claims 1-78, about 38.5 mol % of cholesterol, and about 1.5 mol % of PEG lipid.

87. A lipid nanoparticle comprising: about 57.5-62.5 mol % of the compound of any one of claims 1-78, about 10 mol % of phospholipid, about 26-29 mol % of cholesterol, and about 2-3 mol % of PEG lipid.

88. The lipid nanoparticle of claim 87, comprising: about 60 mol % of the compound of any one of claims 1-78, about 27.5 mol % of cholesterol, and about 2.5 mol % of PEG lipid.

89. A lipid nanoparticle comprising: about 45-50 mol % of the compound of any one of claims 1-78, about 10 mol % of phospholipid, about 37.5-40.5 mol % of cholesterol, and about 3-4 mol % of PEG lipid.

90. The lipid nanoparticle of claim 89, comprising: about 47.5 mol % of the compound of any one of claims 1-38, about 39 mol % of cholesterol, and about 3.5 mol % of PEG lipid.

91. The lipid nanoparticle of any one of claims 80-90, further comprising a targeting component.

92. The lipid nanoparticle of claim 91, wherein the targeting component is a targeting lipid.

93. The lipid nanoparticle of claim 91, wherein the targeting component is an active targeting component.

94. The lipid nanoparticle of claim 91, wherein the active targeting component is a protein, a peptide, a small molecule, or an antibody or antigen-binding fragment thereof.

95. The lipid nanoparticle of any one of claims 79-94, further comprising one or more polynucleotides encapsulated within the lipid nanoparticle.

96. The lipid nanoparticle of claim 95, wherein the one or more polynucleotides comprises RNA.

97. The lipid nanoparticle of claim 95, wherein the one or more polynucleotides comprises DNA.

98. The lipid nanoparticle of claim 95, wherein the one or more polynucleotides comprises DNA and RNA.

99. A pharmaceutical composition comprising the lipid nanoparticle of any one of claims 79-98, and a pharmaceutically acceptable excipient.

100. A pharmaceutical composition comprising the lipid nanoparticle of any one of claims 95-98, and a pharmaceutically acceptable excipient.

101. A method of delivering a polynucleotide to a cell or tissue in a subject, comprising administering to the subject an effective amount of the lipid nanoparticle of any one of claims 79-98 or the pharmaceutical composition of claim 100.

102. The method of claim 101, wherein the cell or tissue comprises an extrahepatic cell or tissue.

103. The method of claim 101, wherein the cell or tissue comprises a brain cell or tissue.

104. The method of claim 101, wherein the cell or tissue comprises a lung cell or tissue.

105. The method of claim 101, wherein the cell or tissue comprises a bone marrow cell or tissue.

106. The method of claim 101, wherein the cell or tissue comprises a spleen cell or tissue.

107. The method of claim 101, wherein the cell or tissue comprises a muscle cell or tissue.

108. The method of claim 101, wherein the cell or tissue comprises a kidney cell or tissue.

109. The method of claim 101, wherein the cell or tissue comprises a heart cell or tissue.

110. The method of claim 101, wherein the cell or tissue comprises a pancreas cell or tissue.

111. The method of claim 101, wherein the cell or tissue comprises an immune cell or tissue.

112. A method of treating a disease in a subject, comprising administering to the subject a therapeutically effective amount of the pharmaceutical composition of claim 99 or claim 100.

113. A method of producing a therapeutic composition, comprising encapsulating an active agent within a lipid nanoparticle, wherein the lipid nanoparticle comprises the compound of any one of claims 1-78.

114. A method of producing a vaccine or prophylactic composition, comprising encapsulating an active agent within a lipid nanoparticle, wherein the lipid nanoparticle comprises the compound of any one of claims 1-78.

115. The method of claim 113 or claim 114, wherein the active agent comprises DNA.

116. The method of claim 113 or claim 114, wherein the active agent comprises RNA.

117. The method of claim 113 or claim 114, wherein the active agent comprises DNA and RNA.

118. The lipid nanoparticle of any one of claims 79-98 or the pharmaceutical composition of claim 99 or claim 100, for use in delivering a polynucleotide to an extrahepatic cell or tissue in a subject.

119. Use of the lipid nanoparticle of any one of claims 79-98 or the pharmaceutical composition of claim 99 or claim 100 in the manufacture of a medicament for delivering a polynucleotide to an extrahepatic cell or tissue in a subject.

120. The lipid nanoparticle of any one of claims 79-98 or the pharmaceutical composition of claim 99 or claim 100 for use in treating a disease in a subject.

121. Use of the lipid nanoparticle of any one of claims 79-98 or the pharmaceutical composition of claim 99 or claim 100 in the manufacture of a medicament for treating a disease in a subject.
